(19) United States
(12) Patent Application Publication
(10) Pub. No.: US 2019/0393424 A1
(43) Pub. Date: Dec. 26, 2019

(54) ORGANIC LIGHT-EMITTING DEVICE

(71) Applicant: Samsung Electronics Co., Ltd., Suwon-si (KR)

(72) Inventors: Sooghang IHN, Hwaseong-si (KR); Myungsun SIM, Suwon-si (KR); Daun Jeong, Suwon-si (KR); Soonok JEON, Suwon-si (KR); Yeonsook CHUNG, Seoul (KR); Sunghan KIM, Seongnam-si (KR); Jongsoo KIM, Seoul (KR); Joonghyuk KIM, Seoul (KR); Hiroshi MIYAZAKI, Yahatanishi-ku (JP); Hasup LEE, Seoul (KR)

(21) Appl. No.: 16/451,830

(22) Filed: Jun. 25, 2019

(30) Foreign Application Priority Data
Jun. 26, 2018 (KR) 10-2018-0073384

(51) Int. Cl. H01L 51/00; C09K 11/06; C07D 403/10

(52) U.S. Cl. CPC H01L 51/0072; C09K 11/06; H01L 51/5012; H01L 51/0067; C07D 403/10

(57) ABSTRACT

An organic light-emitting device comprising: a first electrode; a second electrode facing the first electrode; and an emission layer between the first electrode and the second electrode, wherein the emission layer comprises a host and a thermally activated delayed fluorescence emitter, the host comprises a first material and a second material, the first material and the second material are different from each other, the second material has a dipole moment of about 5.5 debye or more, the second material does not comprise a group represented by *^{=o}, and * indicates a binding site to a neighboring atom.

### CROSS-REFERENCE TO RELATED APPLICATION

This application claims the benefit of Korean Patent Application No. 10-2018-0073384, filed on Jun. 26, 2018, in the Korean Intellectual Property Office, the disclosure of which is incorporated herein in its entirety by reference.

### BACKGROUND

### 1. Field

One or more embodiments relate to an organic light-emitting device.

### 2. Description of the Related Art

Organic light-emitting devices are self-emission devices that produce full-color images, and also have wide viewing angles, high contrast ratios, short response times, and excellent characteristics in terms of brightness, driving voltage, and response speed, compared to devices in the art.

In an example, an organic light-emitting device includes an anode, a cathode, and an organic layer that is disposed between the anode and the cathode and includes an emission layer. A hole transport region may be between the anode and the emission layer, and an electron transport region may be between the emission layer and the cathode. Holes provided from the anode may move toward the emission layer through the hole transport region, and electrons provided from the cathode may move toward the emission layer through the electron transport region. Carriers, such as holes and electrons, recombine in an emission layer region to produce excitons. These excitons transit from an excited state to a ground state, thereby generating light.

### SUMMARY

Aspects of the present disclosure provide an organic light-emitting device having high efficiency and a long lifespan.

Additional aspects will be set forth in part in the description which follows and, in part, will be apparent from the description, or may be learned by practice of the presented embodiments.

An aspect provides an organic light-emitting device comprising: a first electrode; a second electrode facing the first electrode; and an emission layer between the first electrode and the second electrode, wherein the emission layer includes a host and a thermally activated delayed fluorescence emitter, the host includes a first material and a second material, the first material and the second material are different from each other, the second material has a dipole moment of about 5.5 debye or more, the second material does not include a group represented by *^{=o}, and * indicates a binding site to a neighboring atom.

### BRIEF DESCRIPTION OF THE DRAWING

These and/or other aspects will become apparent and more readily appreciated from the following description of the embodiments, taken in conjunction with FIGURE which is a schematic view of an organic light-emitting device according to an exemplary embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

As used herein, the term “and/or” includes any and all combinations of one or more of the associated listed items, and “or” means “and/or”. Expressions such as “at least one of,” when preceding a list of elements, modify the entire list of elements and do not modify the individual elements of the list. The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, 10%, or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

According to one or more exemplary embodiments, an organic light-emitting device includes a first electrode, a second electrode facing the first electrode, and an emission layer between the first electrode and the second electrode.

The emission layer may include a host and a thermally activated delayed fluorescence emitter.

The host may include a first material and a second material, and the first material and the second material may be different from each other.

The first material may be a hole transport material. For example, the first material may not include an electron transport moiety.

For example, the first material may not include a cyano group, a π electron-depleted nitrogen-containing cyclic group, and groups represented by the following Formulae:

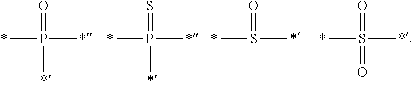

In the formulae, *, *′, and *″ each indicate a binding site to a neighboring atom.

The second material may have a dipole moment of about 5.5 debye or more, for example, in a range of about 5.5 debye to about 20.0 debye.

In one embodiment, the second material may have a dipole moment in a range of about 5.5 debye to about 15.0 debye, but embodiments of the present disclosure are not limited thereto.

The second material does not include a group represented by *^{=o} (wherein * indicates a binding site to a neighboring atom). For example, the second material may not include groups represented by the following formulae:

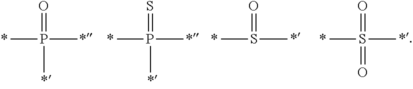

In the formulae, *, *′, and *″ each indicate a binding site to a neighboring atom.

The emission layer may not include a phosphorescence emitter. That is, the emission layer may not include a compound capable of emitting light due to a phosphorescence emission mechanism.

In one embodiment, the first material may include a π electron-rich cyclic group, and may not include an electron transport moiety, and the second material may include a π electron-rich cyclic group and an electron transport moiety, and the electron transport moiety may be a cyano group, a π electron-depleted nitrogen-containing cyclic group, or a combination thereof.

In one or more embodiments, each of the first material and the second material may include a carbazole group.

In one or more embodiments, each of the first material and the second material may include two or more carbazole groups, but embodiments of the present disclosure are not limited thereto.

In one or more embodiments, the second material may include a cyano group (for example, one, two, three, or four cyano groups).

In one or more embodiments, the second material may include an asymmetric compound including a cyano group (for example, one, two, three, or four cyano groups).

In one or more embodiments, the first material may include a cyano group-free benzene group and a cyano group-free carbazole group, the second material may include a cyano group-containing benzene group, a cyano group-containing carbazole group, or a combination thereof.

In one or more embodiments,

an absolute value of a lowest unoccupied molecular orbital (LUMO) energy level of the first material may be in a range of about 0.90 eV to about 1.20 eV,

an absolute value of a highest occupied molecular orbital (HOMO) energy level of the first material may be in a range of about 5.20 eV to about 5.60 eV,

an absolute value of a LUMO energy level of the second material may be in a range of about 1.80 eV to about 2.20 eV, and

an absolute value of a HOMO energy level of the second material may be in a range of about 5.40 eV to about 6.00 eV, but embodiments of the present disclosure are not limited thereto.

When the first material and the second material satisfy the HOMO and LUMO energy level range as described above, charge and/or exciton transfer and energy flow in the emission layer are made smooth, thereby implementing an organic light-emitting device having high luminescent efficiency and a long lifespan.

In one or more embodiments, the first material may include a compound represented by Formula H-1(1), a compound represented by Formula H-1(2), a compound represented by Formula H-1(3), or a combination thereof.

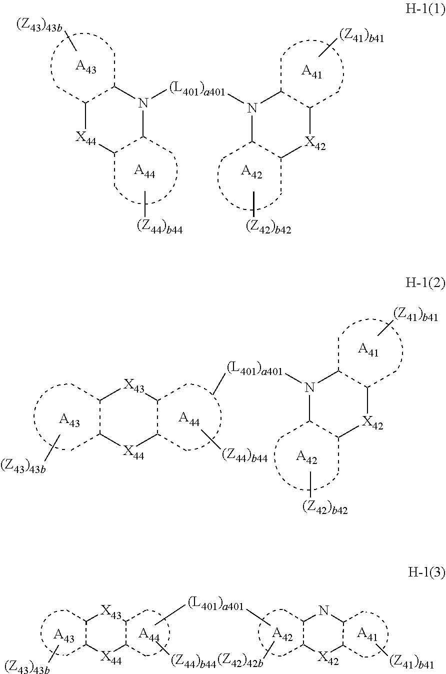

In Formulae H-1(1) to H-1(3), ring A₄₁ to ring A₄₄ may each independently be a benzene group, a naphthalene group, an indene group, an indole group, a benzofuran group, a benzothiophene group, a benzosilole group, a fluorene group, a carbazole group, a dibenzofuran group, a dibenzothiophene group, or a dibenzosilole group.

For example, ring A₄₁ to ring A₄₄ may each independently be a benzene group, a fluorene group, a carbazole group, a dibenzofuran group, a dibenzothiophene group, or a dibenzosilole group, wherein ring A₄₁, ring A₄₂, or a combination thereof may each independently be a benzene group, and ring A₄₃, ring A₄₄, or a combination thereof may each independently be a benzene group.

In Formulae H-1(1) to H-1(3),

X₄₁ may be N-[(L₄₁₁)_{c411}-Z₄₁₁], C(Z₄₁₅)(Z₄₁₆), O, or S,

X₄₂ may be a single bond, N-[(L₄₁₂)_{c412}-Z₄₁₂], C(Z₄₁₇)(Z₄₁₈), O, or S,

X₄₃ may be N-[(L₄₁₃)_{c413}-Z₄₁₃], C(Z₄₁₆)(Z₄₂₀), O, or S, and

X₄₄ may be a single bond, N-[(L₄₁₄)_{c414}-Z₄₁₄], C(Z₄₂₁)(Z₄₂₂), O, or S.

In Formulae H-1(1) to H-1(3), L₄₀₁ and L₄₁₁ to L₄₁₄ may each independently be:

a single bond; or

a π electron-rich cyclic group unsubstituted or substituted with a deuterium, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, a (C₁-C₂₀ alkyl)phenyl group, —Si(Q₄₀₁)(Q₄₀₂)(Q₄₀₃), or a combination thereof (for example, a benzene group, a heptalene group, an indene group, a naphthalene group, an azulene group, an indacene group, acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, a furan group, a thiophene group, a benzofuran group, a benzothiophene group, a benzosilole group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a benzosilolocarbazole group, a triindolobenzene group, an acridine group, or a dihydroacridine group, each unsubstituted or substituted with a deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, —Si(Q₄₀₁)(Q₄₀₂)(Q₄₀₃), or a combination thereof).

In Formulae H-1(1) to H-1(3), a401 and c411 to c414 each indicate the number of L₄₀₁ and the number of L₄₁₁ to L₄₁₄, and may each independently be an integer from 1 to 10 (for example, an integer from 1 to 5). When a401 is two or more, two or more L₄₀₁ may be identical to or different from each other, when c411 is two or more, two or more L₄₁₁ may be identical to or different from each other, when c412 is two or more, two or more L₄₁₂ may be identical to or different from each other, when c413 is two or more, two or more L₄₁₃ may be identical to or different from each other, and when c414 is two or more, two or more L₄₁₄ may be identical to or different from each other.

In Formulae H-1(1) to H-1(3), Z₄₁ to Z₄₄ and Z₄₁₁ to Z₄₂₂ may each independently be:

a hydrogen, a deuterium, a C₁-C₂₀ alkyl group, or a C₁-C₂₀ alkoxy group; or

a π electron-rich cyclic group, unsubstituted or substituted with a deuterium, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, a (C₁-C₂₀ alkyl)phenyl group, —Si(Q₄₀₁)(Q₄₀₂)(Q₄₀₃), or a combination thereof (for example, a phenyl group, a biphenyl group, a terphenyl group, a tetraphenyl group, a naphthyl group, a fluorenyl group, a spiro-bifluorenyl group, a benzofluorenyl group, a dibenzofluorenyl group, a phenanthrenyl group, an anthracenyl group, a triphenylenyl group, a pyrenyl group, a chrysenyl group, a pyrrolyl group, an isoindolyl group, an indolyl group, a furanyl group, a thiophenyl group, a benzofuranyl group, a benzothiophenyl group, a benzosilolyl group, a benzocarbazolyl group, a dibenzocarbazolyl group, a dibenzofuranyl group, a dibenzothiophenyl group, a carbazolyl group, a dibenzosilolyl group, an indenocarbazolyl group, an indolocarbazolyl group, a benzofurocarbazolyl group, a benzothienocarbazolyl group, a benzosilolocarbazolyl group, an acridinyl group, or a dihydroacridinyl group, each unsubstituted or substituted with a deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group —Si(Q₄₀₁)(Q₄₀₂)(Q₄₀₃), or a combination thereof).

In Formulae H-1(1) to H-1(3), b41 to b44 each indicate the number of Z₄₁ to the number of Z₄₄, and may each independently be 1, 2, 3, or 4, and Q₄₀₁ to Q₄₀₃ may each independently be a hydrogen, a deuterium, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, or a tetraphenyl group.

In one embodiment, in Formula H-1(1) to H-1(3),

L₄₀₁ and L₄₁₁ to L₄₁₄ may each independently be:

a single bond; or

a benzene group, a fluorene group, a dibenzofuran group, a dibenzothiophene group, a carbazole group, a dibenzosilole group, an indenocarbazole group, an indolocarbazole group, a benzofurocarbazole group, a benzothienocarbazole group, a benzosilolocarbazole group, an acridine group, or a dihydroacridine group, each unsubstituted or substituted with a deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, a (C₁-C₁₀ alkyl)phenyl group, or a combination thereof,

Z₄₁ to Z₄₄ and Z₄₁₁ to Z₄₂₂ may each independently be:

a hydrogen, a deuterium, a C₁-C₁₀ alkyl group, or a C₁-C₁₀ alkoxy group; or

a phenyl group, a biphenyl group, a terphenyl group, a tetraphenyl group, a fluorenyl group, a dibenzocarbazolyl group, a dibenzofuranyl group, a dibenzothiophenyl group, a carbazolyl group, a dibenzosilolyl group, an indenocarbazolyl group, an indolocarbazolyl group, a benzofurocarbazolyl group, a benzothienocarbazolyl group, a benzosilolocarbazolyl group, an acridinyl group, or a dihydroacridinyl group, each unsubstituted or substituted with a deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, a (C₁-C₁₀ alkyl)phenyl group, or a combination thereof, but embodiments of the present disclosure are not limited thereto.

In one embodiment, the first material may include one of Compounds H1 to H32, or any combination thereof, but embodiments of the present disclosure are not limited thereto:

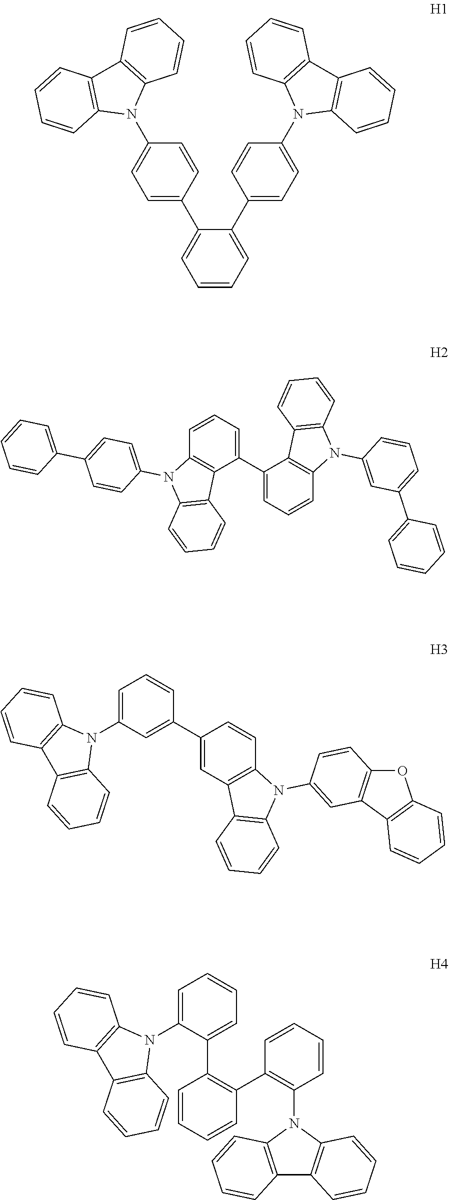 [SMILES: c1ccc(-c2ccc(-n3c4ccccc4c4ccccc43)cc2)c(-c2ccc(-n3c4ccccc4c4ccccc43)cc2)c1 ; c1ccc(-c2ccc(-n3c4ccccc4c4c(-c5cccc6c5c5ccccc5n6-c5cccc(-c6ccccc6)c5)cccc43)cc2)cc1 ; c1cc(-c2ccc3c(c2)c2ccccc2n3-c2ccc3oc4ccccc4c3c2)cc(-n2c3ccccc3c3ccccc32)c1 ; c1ccc(-c2ccccc2-n2c3ccccc3c3ccccc32)c(-c2ccccc2-n2c3ccccc3c3ccccc32)c1]

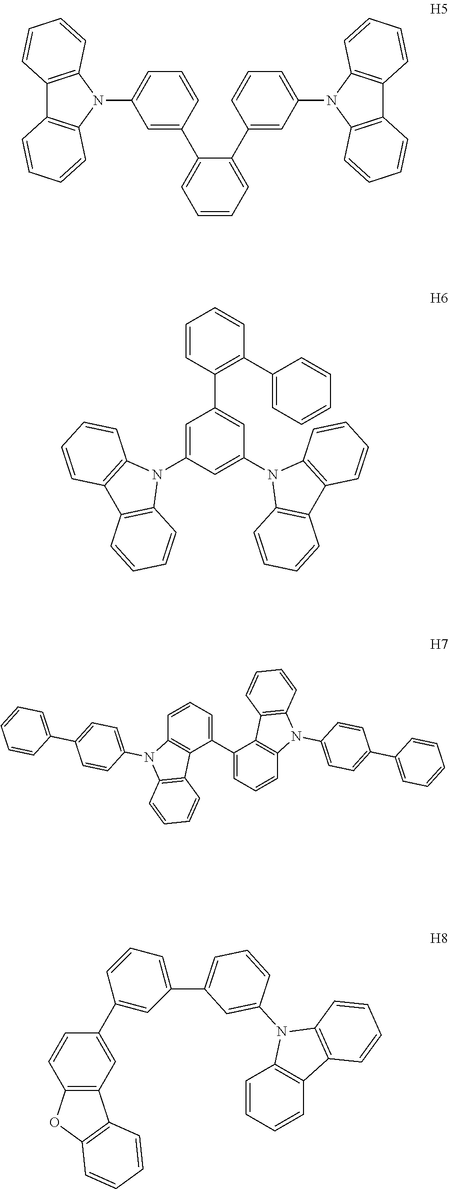 [SMILES: c1cc(-c2ccccc2-c2cccc(-n3c4ccccc4c4ccccc43)c2)cc(-n2c3ccccc3c3ccccc32)c1 ; c1ccc(-c2ccccc2-c2cc(-n3c4ccccc4c4ccccc43)cc(-n3c4ccccc4c4ccccc43)c2)cc1 ; c1ccc(-c2ccc(-n3c4ccccc4c4c(-c5cccc6c5c5ccccc5n6-c5ccc(-c6ccccc6)cc5)cccc43)cc2)cc1 ; c1cc(-c2cccc(-n3c4ccccc4c4ccccc43)c2)cc(-c2ccc3oc4ccccc4c3c2)c1]

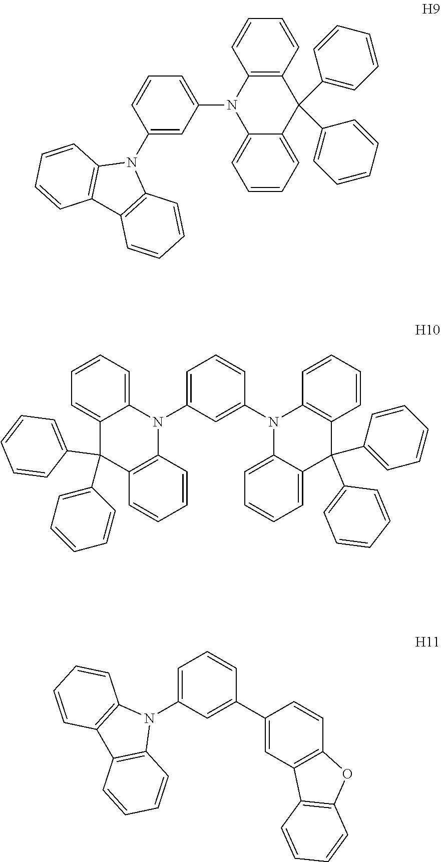 [SMILES: c1ccc(C2(c3ccccc3)c3ccccc3N(c3cccc(-n4c5ccccc5c5ccccc54)c3)c3ccccc32)cc1 ; c1ccc(C2(c3ccccc3)c3ccccc3N(c3cccc(N4c5ccccc5C(c5ccccc5)(c5ccccc5)c5ccccc54)c3)c3ccccc32)cc1 ; c1cc(-c2ccc3oc4ccccc4c3c2)cc(-n2c3ccccc3c3ccccc32)c1]

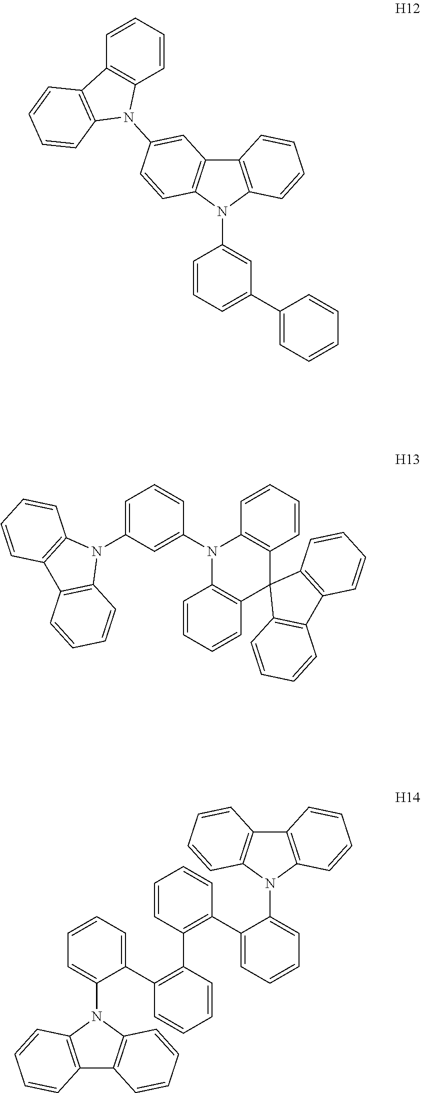 [SMILES: c1ccc(-c2cccc(-n3c4ccccc4c4cc(-n5c6ccccc6c6ccccc65)ccc43)c2)cc1 ; c1cc(N2c3ccccc3C3(c4ccccc4-c4ccccc43)c3ccccc32)cc(-n2c3ccccc3c3ccccc32)c1 ; c1ccc(-c2ccccc2-n2c3ccccc3c3ccccc32)c(-c2ccccc2-c2ccccc2-n2c3ccccc3c3ccccc32)c1]

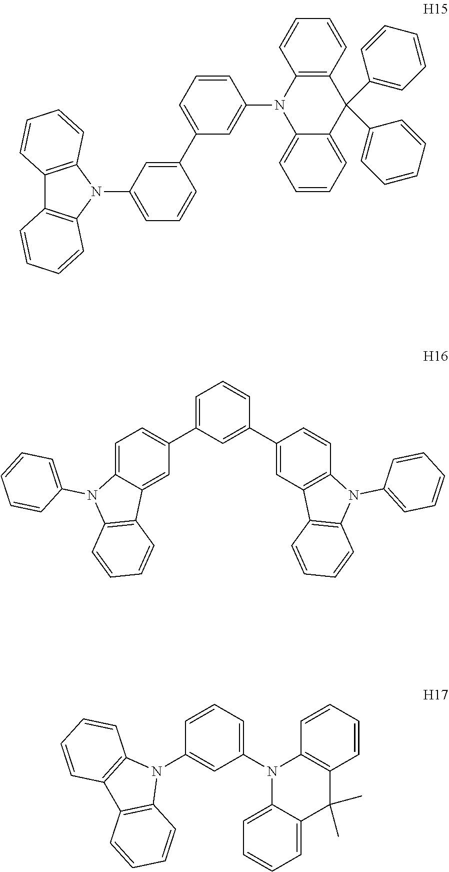 [SMILES: c1ccc(C2(c3ccccc3)c3ccccc3N(c3cccc(-c4cccc(-n5c6ccccc6c6ccccc65)c4)c3)c3ccccc32)cc1 ; c1ccc(-n2c3ccccc3c3cc(-c4cccc(-c5ccc6c(c5)c5ccccc5n6-c5ccccc5)c4)ccc32)cc1 ; CC1(C)c2ccccc2N(c2cccc(-n3c4ccccc4c4ccccc43)c2)c2ccccc21]

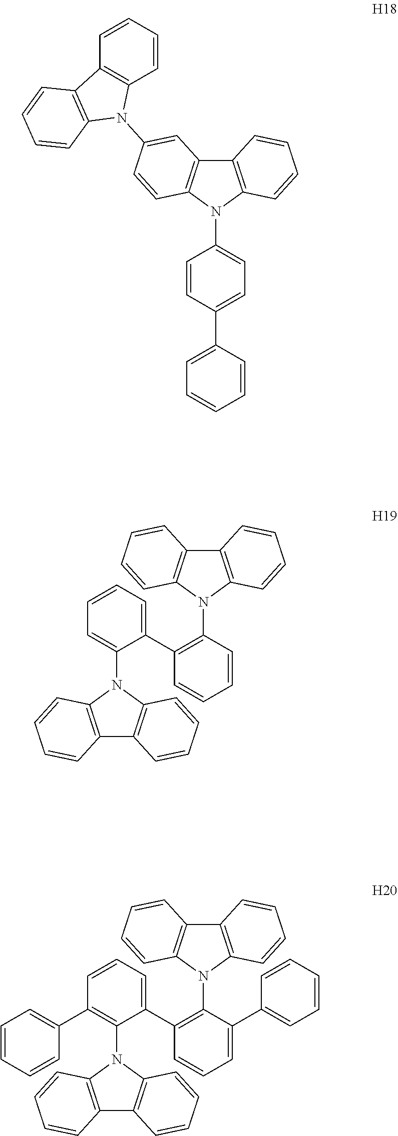 [SMILES: c1ccc(-c2ccc(-n3c4ccccc4c4cc(-n5c6ccccc6c6ccccc65)ccc43)cc2)cc1 ; c1ccc(-n2c3ccccc3c3ccccc32)c(-c2ccccc2-n2c3ccccc3c3ccccc32)c1 ; c1ccc(-c2cccc(-c3cccc(-c4ccccc4)c3-n3c4ccccc4c4ccccc43)c2-n2c3ccccc3c3ccccc32)cc1]

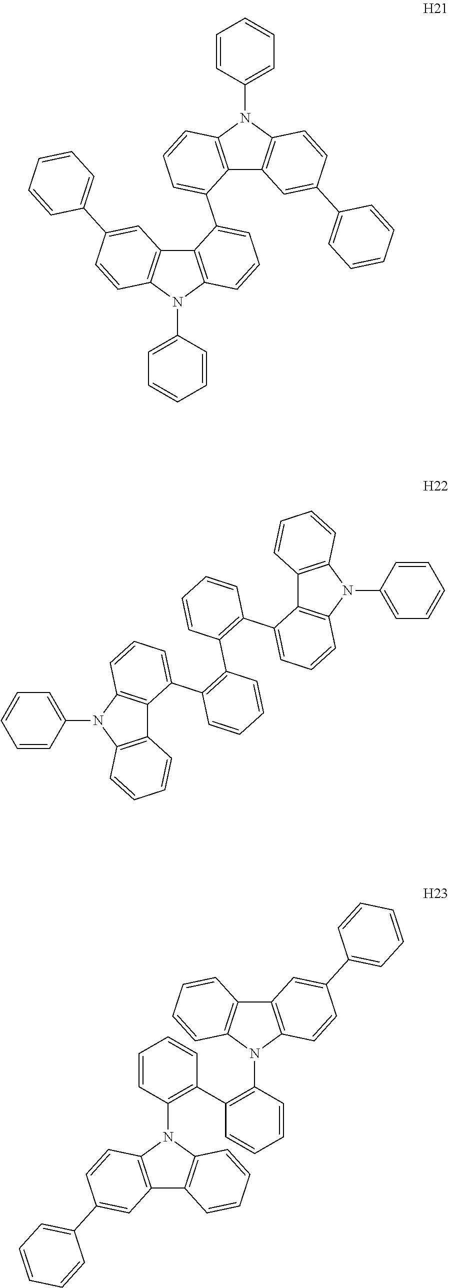 [SMILES: c1ccc(-c2ccc3c(c2)c2c(-c4cccc5c4c4cc(-c6ccccc6)ccc4n5-c4ccccc4)cccc2n3-c2ccccc2)cc1 ; c1ccc(-n2c3ccccc3c3c(-c4ccccc4-c4ccccc4-c4cccc5c4c4ccccc4n5-c4ccccc4)cccc32)cc1 ; c1ccc(-c2ccc3c(c2)c2ccccc2n3-c2ccccc2-c2ccccc2-n2c3ccccc3c3cc(-c4ccccc4)ccc32)cc1]

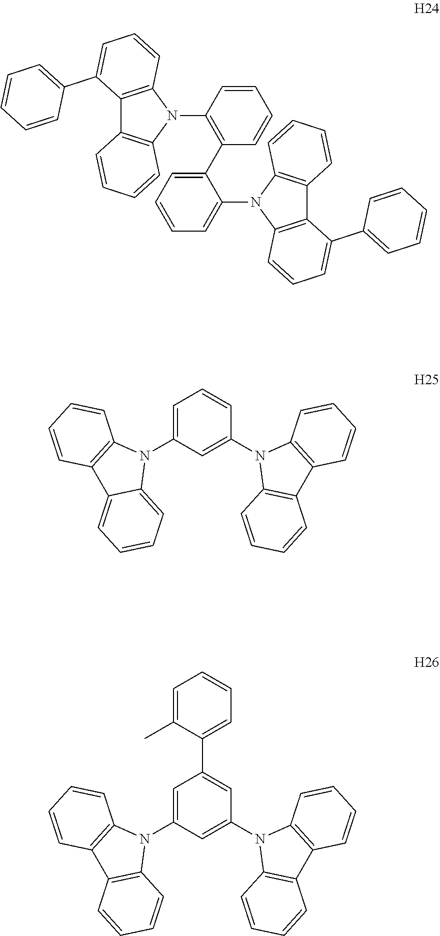 [SMILES: c1ccc(-c2cccc3c2c2ccccc2n3-c2ccccc2-c2ccccc2-n2c3ccccc3c3c(-c4ccccc4)cccc32)cc1 ; c1cc(-n2c3ccccc3c3ccccc32)cc(-n2c3ccccc3c3ccccc32)c1 ; Cc1ccccc1-c1cc(-n2c3ccccc3c3ccccc32)cc(-n2c3ccccc3c3ccccc32)c1]

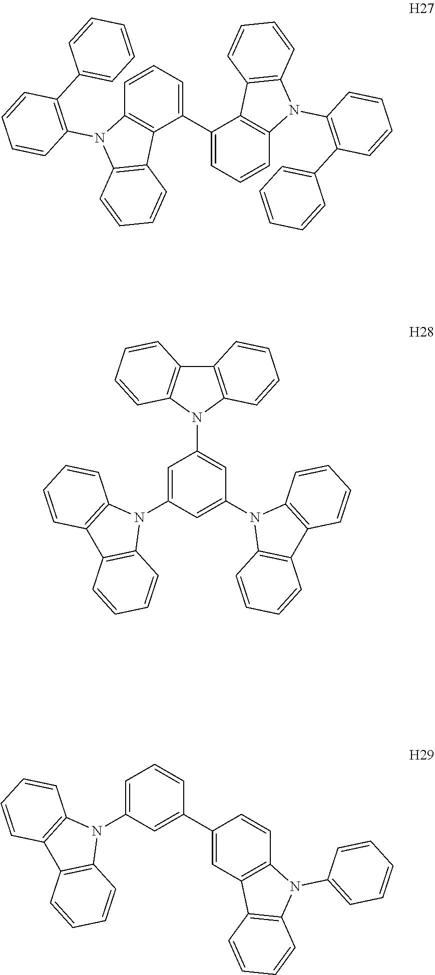 [SMILES: c1ccc(-c2ccccc2-n2c3ccccc3c3c(-c4cccc5c4c4ccccc4n5-c4ccccc4-c4ccccc4)cccc32)cc1 ; c1ccc2c(c1)c1ccccc1n2-c1cc(-n2c3ccccc3c3ccccc32)cc(-n2c3ccccc3c3ccccc32)c1 ; c1ccc(-n2c3ccccc3c3cc(-c4cccc(-n5c6ccccc6c6ccccc65)c4)ccc32)cc1]

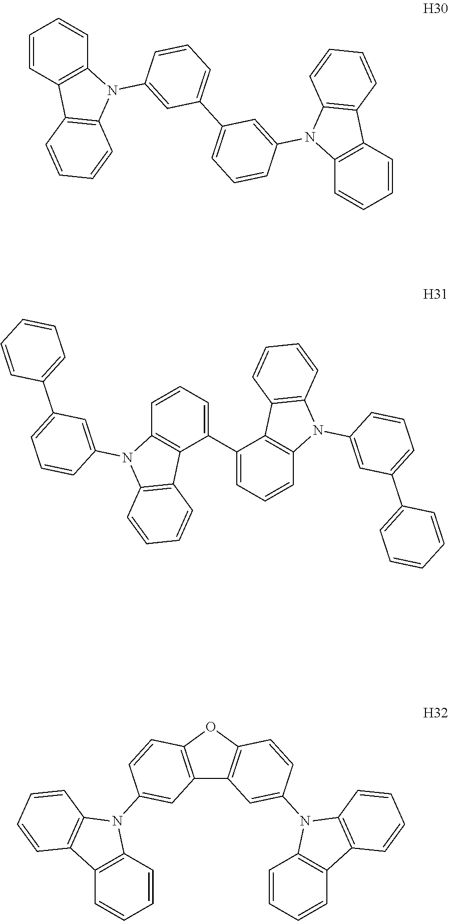 [SMILES: c1cc(-c2cccc(-n3c4ccccc4c4ccccc43)c2)cc(-n2c3ccccc3c3ccccc32)c1 ; c1ccc(-c2cccc(-n3c4ccccc4c4c(-c5cccc6c5c5ccccc5n6-c5cccc(-c6ccccc6)c5)cccc43)c2)cc1 ; c1ccc2c(c1)c1ccccc1n2-c1ccc2oc3ccc(-n4c5ccccc5c5ccccc54)cc3c2c1]

In one embodiment, the first material may not be an amine group-containing compound.

In one or more embodiments, the first material may not be 1,3-bis(9-carbazolyl)benzene (mCP), tris(4-carbazoyl-9-ylphenyl)amine (TCTA), 4,4′-bis(N-carbazolyl)-1,1′-biphenyl (CBP), 3,3-bis(carbazol-9-yl)biphenyl (mCBP), N,N′-di(1-naphthyl)-N,N′-diphenyl-(1,1′-biphenyl)-4,4′-diamine (NPB), 4,4′,4″-tris[phenyl(m-tolyl)amino]triphenylamine (m-MTDATA), or N,N′-bis(3-methylphenyl)-N,N′ -diphenylbenzidine (TPD).

In one or more embodiments, the second material may include a compound represented by Formula E-1:

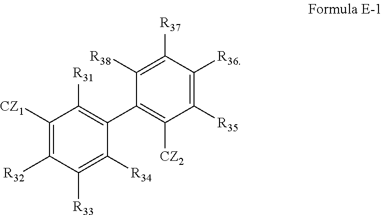

In Formula E-1, CZ₁ and CZ₂ may each independently be a group represented by Formulae 2-1 or 2-2.

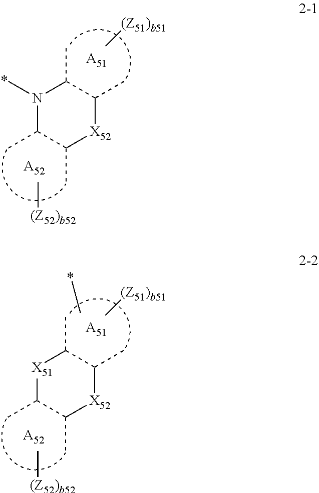

In Formulae 2-1 and 2-2, ring A₅₁ and ring A₅₂ may each independently be a benzene group, a naphthalene group, an indene group, an indole group, a benzofuran group, a benzothiophene group, a benzosilole group, a fluorene group, a carbazole group, a dibenzofuran group, a dibenzothiophene group, a dibenzosilole group, a pyridine group, a pyrimidine group, a pyrazine group, a pyridazine group, a triazine group, a quinoline group, an isoquinoline group, a quinoxaline group, a quinazoline group, a pyrazole group, an imidazole group, a triazole group, an oxazole group, an isoxazole group, a thiazole group, an isothiazole group, an oxadiazole group, a thiadiazole group, an azaindene group, an azaindole group, an azabenzofuran group, an azabenzothiophene group, an azabenzosilole group, an azafluorene group, an azacarbazole group, an azadibenzofuran group, an azadibenzothiophene group, or an azadibenzosilole group.

For example, ring A₅₁ and ring A₅₂ may each independently be a benzene group, a fluorene group, a carbazole group, a dibenzofuran group, a dibenzothiophene group, or a dibenzosilole group, but embodiments of the present disclosure are not limited thereto.

In Formula 2-2, X₅₁ may be N-[(L₅₁₁)_{c511}-Z₅₁₁], C(Z₅₁₃)(Z₅₁₄), O, or S, and in Formulae 2-1 and 2-2, X₅₂ may be a single bond, N-[(L₅₁₂)_{c512}-Z₅₁₂], C(Z₅₁₅)(Z₅₁₆), O, or S.

For example, in Formula 2-2, X₅₁ may be N-[(L₅₁₁)_{c511}-Z₅₁₁], and in Formulae 2-1 and 2-2, X₅₂ may be a single bond, but embodiments of the present disclosure are not limited thereto.

In Formula 2-2, i) X₅₁ may be N-[(L₅₁₁)_{c511}-Z₅₁₁], or ii) ring A₅₁, ring A₅₂, or a combination thereof may be an indole group, a carbazole group, an azaindole group, or an azacarbazole group, and

L₅₁₁ and L₅₁₂ may each independently be a single bond, a substituted or unsubstituted C₃-C₁₀ cycloalkylene group, a substituted or unsubstituted C₂-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₂-C₆₀ heteroarylene group, a substituted or unsubstituted divalent non-aromatic condensed polycyclic group, or a substituted or unsubstituted divalent non-aromatic condensed heteropolycyclic group.

L₅₁₁ and L₅₁₂ may each independently have the same definition as that of L₃.

In one embodiment, c511 and c512 each indicate the number of L₅₁₁ and the number of L₅₁₂, and may each independently be an integer from 1 to 10 (for example, an integer from 1 to 5). When c511 is two or more, two or more L₅₁₁ may be identical to or different from each other, and when c512 is two or more, two or more L₅₁₂ may be identical to or different from each other.

In one embodiment, Z₃₁ to Z₃₈, Z₅₁, Z₅₂, and Z₅₁₁ to Z₅₁₆ may each independently be:

a hydrogen, a deuterium or a cyano group (CN); or

a C₁-C₂₀ alkyl group, a phenyl group, a biphenyl group, a terphenyl group, a dibenzofuranyl group, or a dibenzothiophenyl group, each unsubstituted or substituted with a deuterium, a cyano group, a C₁-C₂₀ alkyl group, a phenyl group, a biphenyl group, or a combination thereof.

In Formulae 2-1 and 2-2, * indicates a binding site to a neighboring atom.

The number of the cyano groups included in Formula E-1 may be at least 1, for example, 1, 2, 3, or 4.

In one embodiment, in Formula E-1, CZ₁ and CZ₂ may each independently be a group represented by Formulae 2(1) to 2(3):

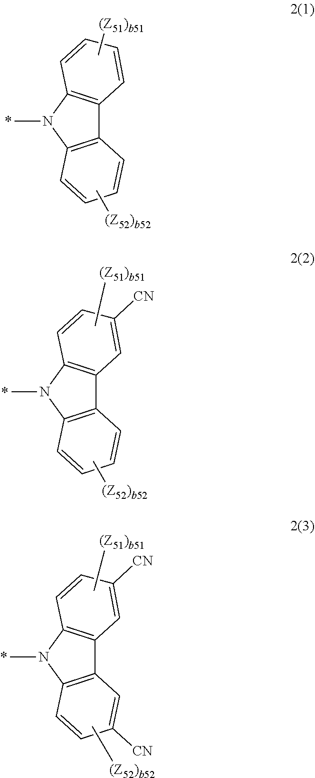

In Formulae 2(1) to 2(3), Z₅₁ and Z₅₂ may each independently have the same definition as described above, and b51 and b52 may each independently be 0, 1, or 2, and * indicates a binding site to a neighboring atom.

For example, in Formulae 2(1) to 2(3), Z₅₁ and Z₅₂ may not each independently be a cyano group.

In one or more embodiments, in Formula E-1, at least one of R₃₁ to R₃₈ may be a cyano group.

In one or more embodiments, in Formula E-1, i) at least one of R₃₁ to R₃₈ may be a cyano group; ii) CZ₁ and CZ₂ may each independently be a group represented by Formulae 2(2) or 2(3); or iii) at least one of R₃₁ to R₃₈ may be a cyano group, and CZ₁ and CZ₂ may each independently be a group represented by Formulae 2(2) or 2(3).

For example, the second material may include one of Compounds E1 to E45, or any combination thereof, but embodiments of the present disclosure are not limited thereto:

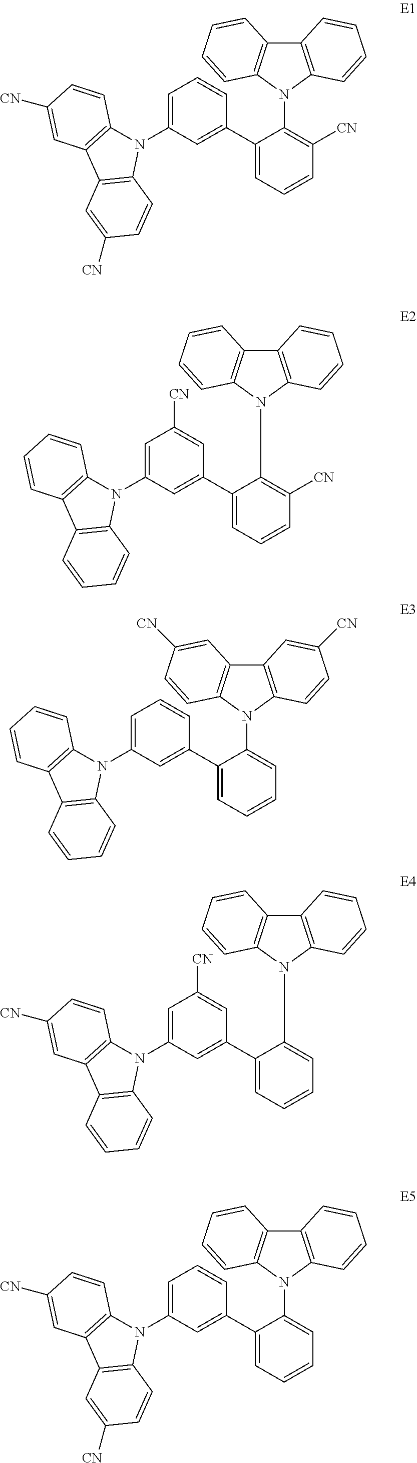

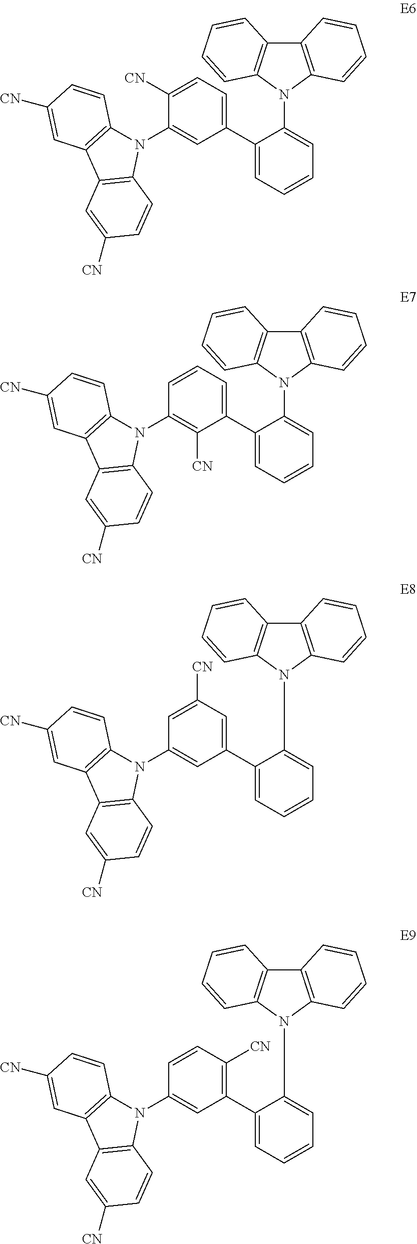

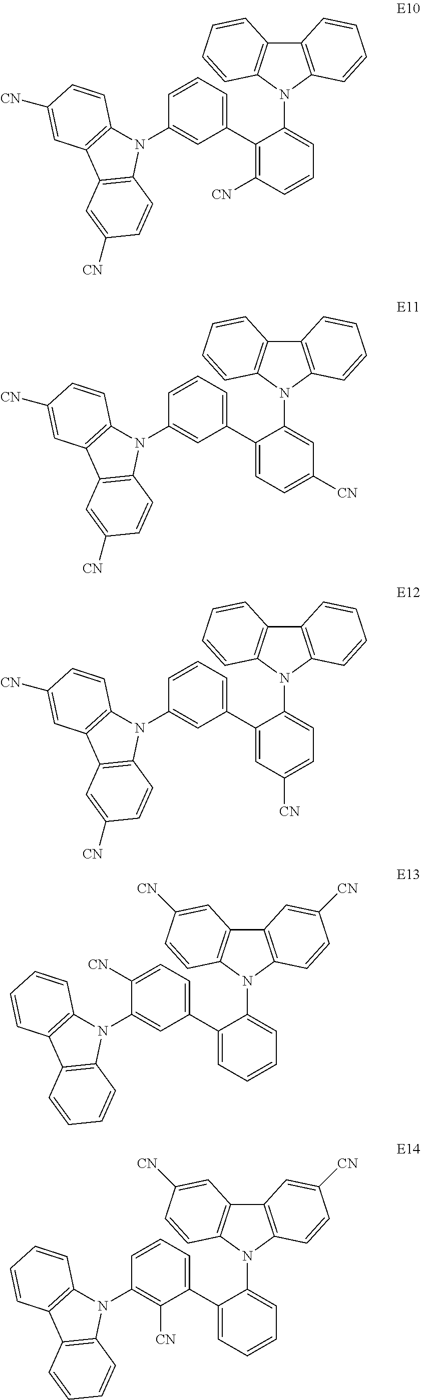

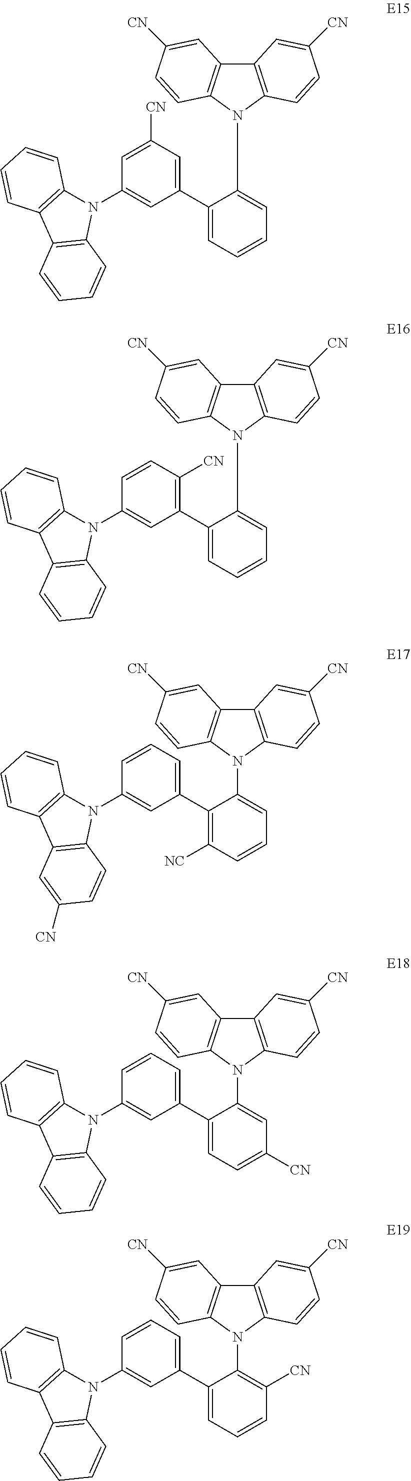

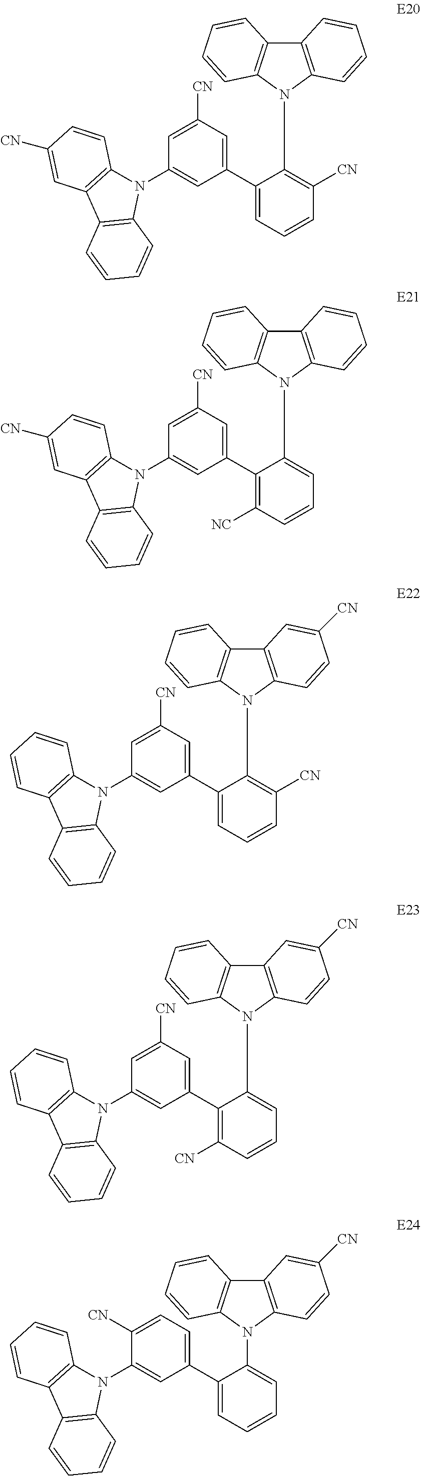

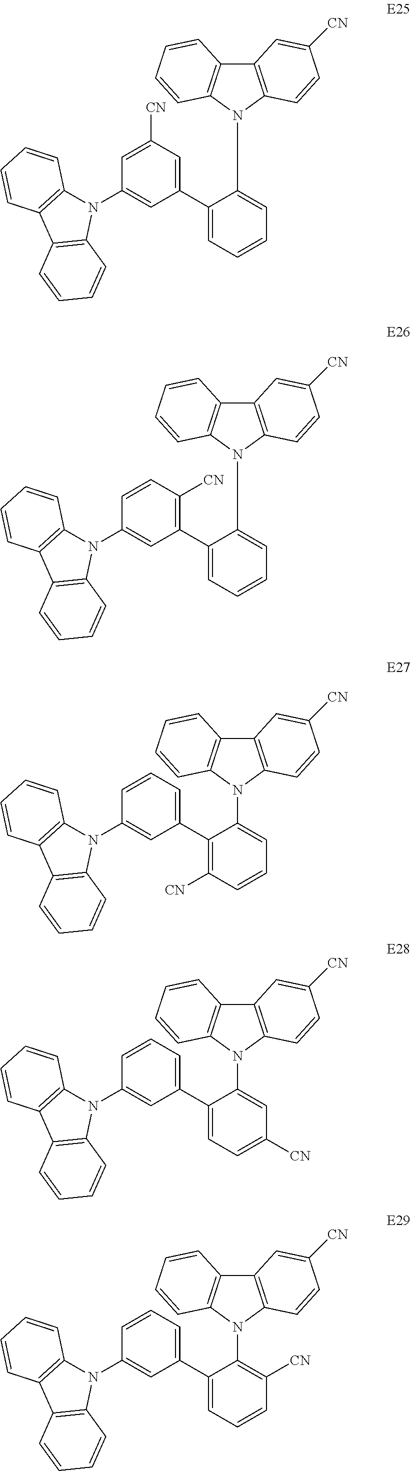

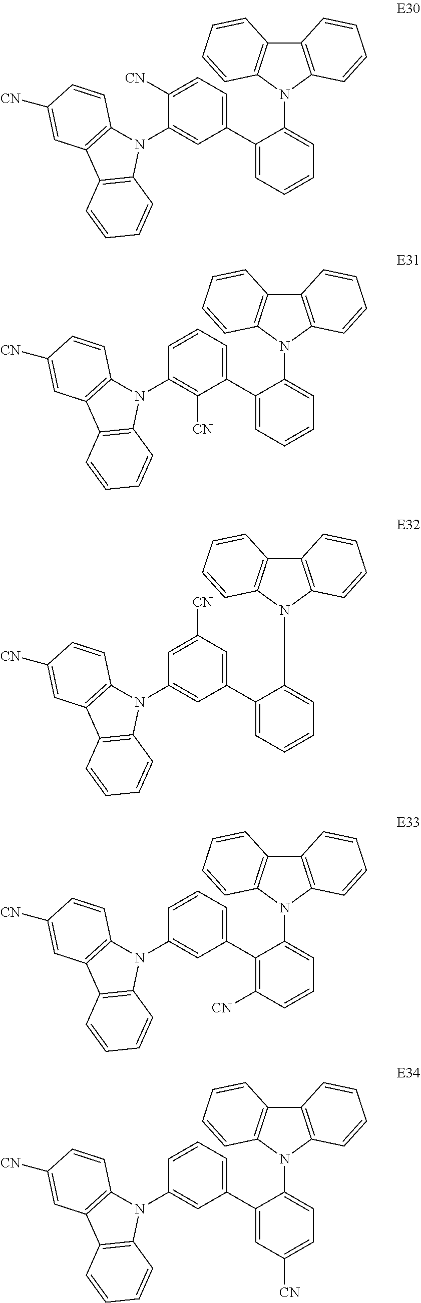

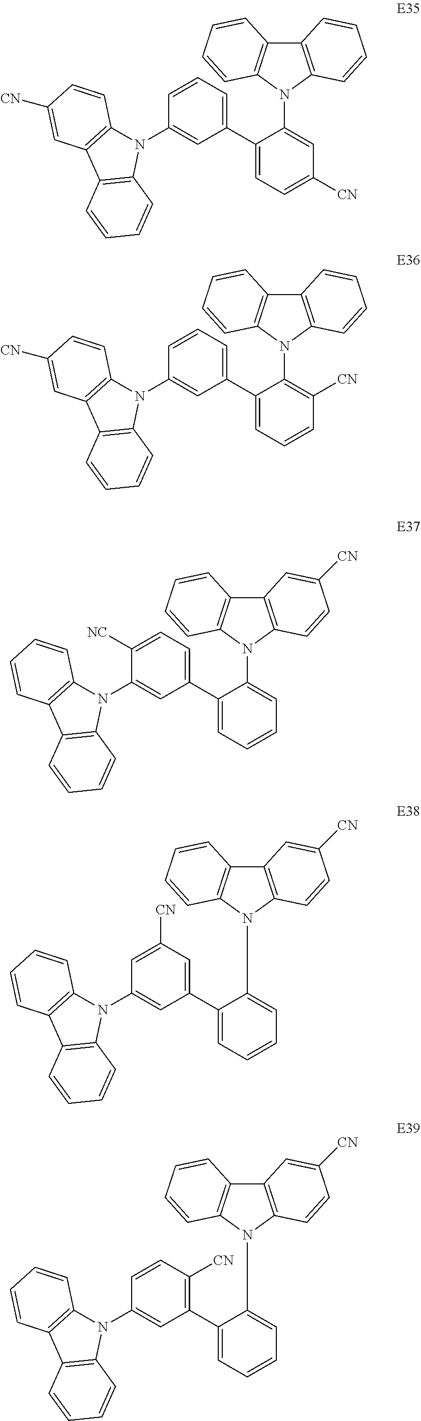

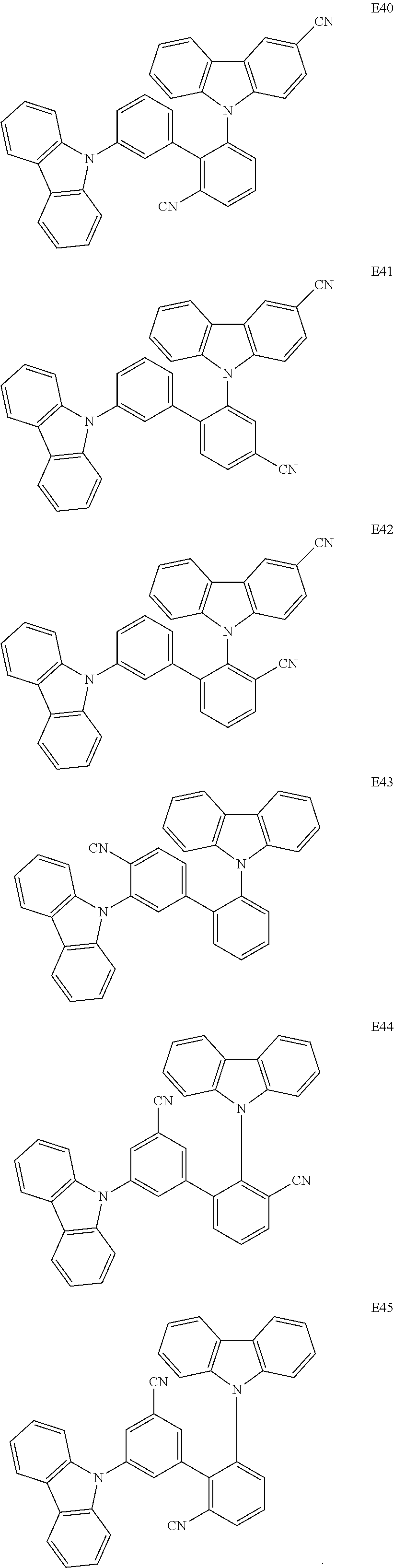

The host includes the first material and the second material that are different from each other, the second material has a dipole moment of about 5.5 debye or more, and the second material does not include a group represented by *^{=o} (wherein * indicates a binding site to a neighboring atom).

The second material has a high dipole moment and does not include a group represented by *^{=o} (wherein * indicates a binding site to a neighboring atom) (for example, a phosphine oxide group-containing compound includes a group represented by *^{=o}). Therefore, when the second material is used in the emission layer together with the first material different from the second material (for example, not including an electron transport moiety), a charge transfer excited state of the thermally activated delayed fluorescence emitter may be stabilized by the second material having a high dipole moment, thereby significantly increasing the delayed fluorescence component of the emission layer. Therefore, since the emission layer including the first material, the second material, and the thermally activated delayed fluorescence emitter is employed, it is possible to achieve high luminescent efficiency and substantially prevent the material for the emission layer from being decomposed by *^{=o} in storing and/or driving the organic light-emitting device, thereby achieving high luminescent efficiency and a long lifespan at the “same time”.

The dipole moment of the second material may be evaluated by calculating electrostatic potential fitting (ESP) charge and interatomic distance of each atom of the corresponding compound was calculated by using a density functional theory (DFT) method of Jaguar program (structurally optimized at a level of B3LYP, 6-31G(d,p)) and calculating the dipole moment of the corresponding compound therefrom.

A difference between a triplet energy level (eV) of the host and a triplet energy level (eV) of the thermally activated delayed fluorescence emitter may be in a range of about 0.2 electron volts (eV) to about 0.5 eV. When the difference between the triplet energy level (eV) of the host and the triplet energy level (eV) of the thermally activated delayed fluorescence emitter is within this range, energy of triplet excitons generated by the thermally activated delayed fluorescence emitter is prevented from leaking to the host in the emission layer. Therefore, it is possible to achieve efficient light emission and suppress the activated excitation energy level of the host, thereby implementing the long lifespan driving of the organic light-emitting device.

The triplet energy level was evaluated by using a DFT method of Gaussian program structurally optimized at a level of B3LYP/6-31G(d,p).

The thermally activated delayed fluorescence emitter may be any compound that may emit delayed fluorescent light according to a thermally delayed fluorescence emission mechanism.

In one embodiment, the difference between the triplet energy level (eV) of the thermally activated delayed fluorescence emitter and the singlet energy level (eV) of the thermally activated delayed fluorescence emitter may be in a range of about 0 eV to about 0.5 eV. When the difference between the triplet energy level (eV) of the thermally activated delayed fluorescence emitter and the singlet energy level (eV) of the thermally activated delayed fluorescence emitter is within this range, up-conversion from the triplet state to the singlet state may be effectively achieved, and the fluorescent dopant may emit a delayed fluorescent light with high luminescent efficiency.

The triplet energy level and the singlet energy level were evaluated by using a DFT method structurally optimized at a level of B3LYP/6-31G(d,p), for example, using Gaussian program.

In one embodiment, the thermally activated delayed fluorescence emitter may include a compound represented by Formula 11:

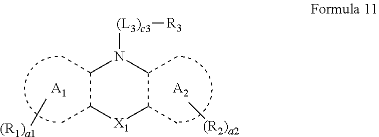

In Formula 11, X₁, may be a single bond, N-[(L₄)_{c4}-R₄], C(R₅)(R₆), O, or S.

For example, X₁ may be a single bond, but embodiments of the present disclosure are not limited thereto.

In Formula 11, A₁ and A₂ may each independently be a benzene group, a naphthalene group, an indene group, an indole group, a benzofuran group, a benzothiophene group, a benzosilole group, a fluorene group, a carbazole group, a dibenzofuran group, a dibenzothiophene group, a dibenzosilole group, an indolofluorene group, an indolocarbazole group, an indolodibenzofuran group, an indolodibenzothiophene group, an indenofluorene group, an indenocarbazole group, an indenodibenzofuran group, an indenodibenzothiophene group, a benzofuranofluorene group, a benzofuranocarbazole group, a benzofuranodibenzofuran group, a benzofuranodibenzothiophene group, a benzothienofluorene group, a benzothienocarbazole group, a benzothienodibenzofuran group, or a benzothienodibenzothiophene group.

For example, A₁ and A₂ may each independently be a benzene group, a fluorene group, a carbazole group, a dibenzofuran group, a dibenzothiophene group, or a dibenzosilole group, wherein A₁, A₂, or a combination thereof may each independently be a benzene group, but embodiments of the present disclosure are not limited thereto.

In one embodiment, L₃ and L₄ may each independently be a single bond, a substituted or unsubstituted C₃-C₁₀ cycloalkylene group, a substituted or unsubstituted C₂-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₂-C₆₀ heteroarylene group, a substituted or unsubstituted divalent non-aromatic condensed polycyclic group, and a substituted or unsubstituted divalent non-aromatic condensed heteropolycyclic group.

For example, L₃ and L₄ may each independently be:

a single bond, a phenylene group, a naphthylene group, a fluorenylene group, a pyridinylene group, a pyrimidinylene group, a pyrazinylene group, a pyridazinylene group, a triazinylene group, a quinolinylene group, an isoquinolinylene group, a carbazolylene group, a dibenzofuranylene group, a dibenzothiophenylene group, or an indolocarbazolylene group; or

a phenylene group, a naphthylene group, a fluorenylene group, a pyridinylene group, a pyrimidinylene group, a pyrazinylene group, a pyridazinylene group, a triazinylene group, a quinolinylene group, an isoquinolinylene group, a carbazolylene group, a dibenzofuranylene group, a dibenzothiophenylene group, and an indolocarbazolylene group, each substituted with a deuterium, a C₁-C₁₀ alkyl group, a C₁-C₁₀ alkoxy group, a phenyl group, a biphenyl group, a terphenyl group, a naphthyl group, a fluorenyl group, a pyridinyl group, a pyrimidinyl group, a pyrazinyl group, a pyridazinyl group, a triazinyl group, a quinolinyl group, an isoquinolinyl group, a carbazolyl group, a dibenzofuranyl group, a dibenzothiophenyl group, an indolocarbazolyl group, —Si(Q₃₁)(Q₃₂)(Q₃₃), —N(Q₃₄)(Q₃₅), or a combination thereof, and Q₃₁ to Q₃₅ may each independently be a C₁-C₁₀ alkyl group, a C₁-C₁₀ alkoxy group, a phenyl group, a biphenyl group, a terphenyl group, or a naphthyl group. However, embodiments of the present disclosure are not limited thereto.

In one embodiment, in Formula 11, L₃ may be a group represented by Formula L-1 or L-2, but embodiments of the present disclosure are not limited thereto:

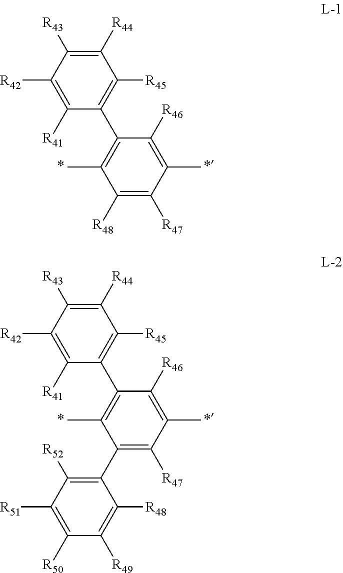

In Formulae L-1 and L-2, R₄₁ to R₅₂ may each independently be a hydrogen, a deuterium, a C₁-C₁₀ alkyl group, a C₁-C₁₀ alkoxy group, a phenyl group, a biphenyl group, a terphenyl group, a naphthyl group, a fluorenyl group, a pyridinyl group, a pyrimidinyl group, a pyrazinyl group, a pyridazinyl group, a triazinyl group, a quinolinyl group, an isoquinolinyl group, a carbazolyl group, a dibenzofuranyl group, a dibenzothiophenyl group, an indolocarbazolyl group, —Si(Q₃₁)(Q₃₂)(Q₃₃), or —N(Q₃₄)(Q₃₅), wherein Q₃₁ to Q₃₅ may each independently have the same definition as described above, * indicates a binding site to a neighboring atom, and *′ indicates a binding site to neighboring L₃ or neighboring R₃.

In one embodiment, c3 and c4 each indicate the number of L₃ and the number of L₄, and may each independently be an integer from 1 to 4. When c3 is two or more, two or more L₃ may be different from or identical to each other, and when c4 is two or more, two or more L₄ may be different from or identical to each other. For example, c3 and c4 may each independently be 1 or 2, but embodiments of the present disclosure are not limited thereto.

In Formula 11, R₁ to Re may each independently be a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), or —B(Q₆)(Q₇), wherein Q₁ to Q, may each independently have the same definition as described above.

In one embodiment, in Formula 11, R₃ may include a π electron-depleted nitrogen-containing cyclic group.

The term “π electron-depleted nitrogen-containing cyclic group” as used herein refers to a group including a cyclic group having at least one *—N=*′ moiety, and examples thereof include an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a benzoisoquinoline group, a phthalazine group, a naphthyridine group, a quinoxaline group, a benzoquinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azaindene group, an azaindole group, an azabenzofuran group, an azabenzothiophene group, an azabenzosilole group, an azafluorene group, an azacarbazole group, an azadibenzofuran group, an azadibenzothiophene group, or an azadibenzosilole group.

In one or more embodiments, R₃ in Formula 11 may be:

a phenyl group, an indenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a pyrrolyl group, an isoindolyl group, an indolyl group, a furanyl group, a thiophenyl group, a silolyl group, a benzofuranyl group, a benzothiophenyl group, a benzosilolyl group, a carbazolyl group, a dibenzofuranyl group, a dibenzothiophenyl group, a dibenzosilolyl group, an indenocarbazolyl group, an indolocarbazolyl group, a benzofuracarbazolyl group, a benzothienocarbazolyl group, a benzosilolocarbazolyl group, an imidazolyl group, a pyrazolyl group, a thiazolyl group, an isothiazolyl group, an oxazolyl group, an isoxazolyl group, a pyridinyl group, a pyrazinyl group, a pyridazinyl group, a pyrimidinyl group, an indazolyl group, a purinyl group, a quinolinyl group, an isoquinolinyl group, a benzoquinolinyl group, a benzoisoquinolinyl group, a phthalazinyl group, a naphthyridinyl group, a quinoxalinyl group, a benzoquinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an oxadiazolyl group, a triazinyl group, a thiadiazolyl group, an imidazopyridinyl group, an imidazopyrimidinyl group, an azaindenyl group, an azaindolyl group, an azabenzofuranyl group, an azabenzothiophenyl group, an azabenzosilolyl group, an azafluorenyl group, an azacarbazolyl group, an azadibenzofuranyl group, an azadibenzothiophenyl group, or an azadibenzosilolyl group,

each unsubstituted or substituted with a deuterium, a C₁-C₁₀ alkyl group, a C₁-C₁₀ alkoxy group, a phenyl group, a (C₁-C₁₀ alkyl)phenyl group, a di(C₁-C₁₀ alkyl)phenyl group, a biphenyl group, a terphenyl group, a di(phenyl)phenyl group, a di(biphenyl)phenyl group, a (pyridinyl)phenyl group, a di(pyridinyl)phenyl group, a (pyrimidinyl)phenyl group, a di(pyrimidinyl)phenyl group, a (triazinyl)phenyl group, a di(triazinyl)phenyl group, a pyridinyl group, a (C₁-C₁₀ alkyl)pyridinyl group, a di(C₁-C₁₀ alkyl)pyridinyl group, a (phenyl)pyridinyl group, a di(phenyl)pyridinyl group, a (biphenyl)pyridinyl group, a di(biphenyl)pyridinyl group, a (terphenyl)pyridinyl group, a bi(terphenyl)pyridinyl group, a (pyridinyl)pyridinyl group, a di(pyridinyl)pyridinyl group, a (pyrimidinyl)pyridinyl group, a di(pyrimidinyl)pyridinyl group, a (triazinyl)pyridinyl group, a di(triazinyl)pyridinyl group, a pyrimidinyl group, a (C₁-C₁₀ alkyl)pyrimidinyl group, a di(C₁-C₁₀ alkyl)pyrimidinyl group, a (phenyl)pyrimidinyl group, a di(phenyl)pyrimidinyl group, a (biphenyl)pyrimidinyl group, a di(biphenyl)pyrimidinyl group, a (terphenyl)pyrimidinyl group, a bi(terphenyl)pyrimidinyl group, a (pyridinyl)pyrimidinyl group, a di(pyridinyl)pyrimidinyl group, a (pyrimidinyl)pyrimidinyl group, a di(pyrimidinyl)pyrimidinyl group, a (triazinyl)pyrimidinyl group, a di(triazinyl)pyrimidinyl group, a triazinyl group, a (C₁-C₁₀ alkyl)triazinyl group, a di(C₁-C₁₀ alkyl)triazinyl group, a (phenyl)triazinyl group, a di(phenyl)triazinyl group, a (biphenyl)triazinyl group, a di(biphenyl)triazinyl group, a (terphenyl)triazinyl group, a bi(terphenyl)triazinyl group, a (pyridinyl)triazinyl group, a di(pyridinyl)triazinyl group, a (pyrimidinyl)triazinyl group, a di(pyrimidinyl)triazinyl group, a (triazinyl)triazinyl group, a di(triazinyl)triazinyl group, a fluorenyl group, a di(C₁-C₁₀ alkyl) a fluorenyl group, a di(phenyl)fluorenyl group, a di(biphenyl)fluorenyl group, a carbazolyl group, a (C₁-C₁₀ alkyl)carbazolyl group, a (phenyl)carbazolyl group, a (biphenyl)carbazolyl group, a dibenzofuranyl group, a (C₁-C₁₀ alkyl)dibenzofuranyl group, a (phenyl)dibenzofuranyl group, a (biphenyl)dibenzofuranyl group, a dibenzothiophenyl group, a (C₁-C₁₀ alkyl)dibenzothiophenyl group, a (phenyl)dibenzothiophenyl group, a (biphenyl)dibenzothiophenyl group, or a combination thereof, but embodiments of the present disclosure are not limited thereto.

In one or more embodiments, R₃ in Formula 11 may be:

a group represented by Formula 13(1) or 13(2);

a phenyl group, a biphenyl group, a terphenyl group, a naphthyl group, a fluorenyl group, a quinolinyl group, an isoquinolinyl group, a carbazolyl group, a dibenzofuranyl group, a dibenzothiophenyl group, or an indolocarbazolyl group; or

a phenyl group, a biphenyl group, a terphenyl group, a naphthyl group, a fluorenyl group, a quinolinyl group, an isoquinolinyl group, a carbazolyl group, a dibenzofuranyl group, a dibenzothiophenyl group, and an indolocarbazolyl group, each substituted with a deuterium, a C₁-C₁₀ alkyl group, a C₁-C₁₀ alkoxy group, a phenyl group, a biphenyl group, a terphenyl group, a naphthyl group, a fluorenyl group, a pyridinyl group, a pyrimidinyl group, a pyrazinyl group, a pyridazinyl group, a triazinyl group, a quinolinyl group, an isoquinolinyl group, a carbazolyl group, a dibenzofuranyl group, a dibenzothiophenyl group, an indolocarbazolyl group, —Si(Q₃₁)(Q₃₂)(Q₃₃), or a combination thereof, and

Q₃₁ to Q₃₃ may each independently be a C₁-C₁₀ alkyl group, a C₁-C₁₀ alkoxy group, a phenyl group, a biphenyl group, a terphenyl group, or a naphthyl group.

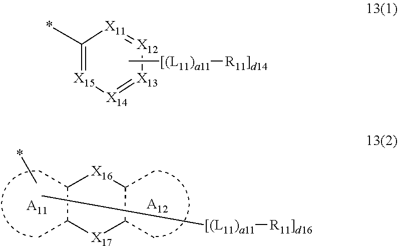

In Formula 13(1), X₁₁ to X₁₅ may each independently be C or N, wherein at least one of X₁₁ to X₁₅ may be N.

For example, two or three of X₁₁ to X₁₅ may be N.

In Formula 13(2), A₁₁ and A₁₂ may each independently be a benzene group, a naphthalene group, a pyridine group, a pyrazine group, a pyrimidine group, a pyridazine group, a quinoline group, an isoquinoline group, a quinoxaline group, or a quinazoline group, and A₁₁, A₁₂, or a combination thereof may be a pyridine group, a pyrazine group, a pyrimidine group, a pyridazine group, a quinoline group, an isoquinoline group, a quinoxaline group, or a quinazoline group.

For example, A₁₁ may be a pyridine group, a pyrimidine group, a quinoline group, an isoquinoline group, a quinoxaline group, or a quinazoline group, and A₁₂ may be a benzene group, or a naphthalene group, but embodiments of the present disclosure are not limited thereto.

In Formula 13(2), X₁₆ may be N-[(L₁₂)ₐ₁₂-R₁₂], C(R₁₄)(R₁₅), O, or S, and X₁₇ may be a single bond, N-[(L₁₃)ₐ₁₃-R₁₃], C(R₁₆)(R₁₇), O, or S.

For example, X₁₆ may be O or S, and X₁₇ may be a single bond, but embodiments of the present disclosure are not limited thereto.

In Formulae 13(1) and 13(2), L₁₁ to L₁₃ may each independently have the same definition as that of L₃, a11 to a13 may each independently have the same definition as that of c3, and R₁₁ to R₁₇ may each independently have the same definition as that of R₁.

In Formula 13(2), d16 may be an integer from 0 to 6, and in Formula 13(1), d14 may be an integer from 0 to 4.

In Formulae 13(1) and 13(2), * indicates a binding site to a neighboring atom.

In one embodiment, R₃ in Formula 11 may be a group represented by Formulae 13-1 to 13-20, but embodiments of the present disclosure are not limited thereto:

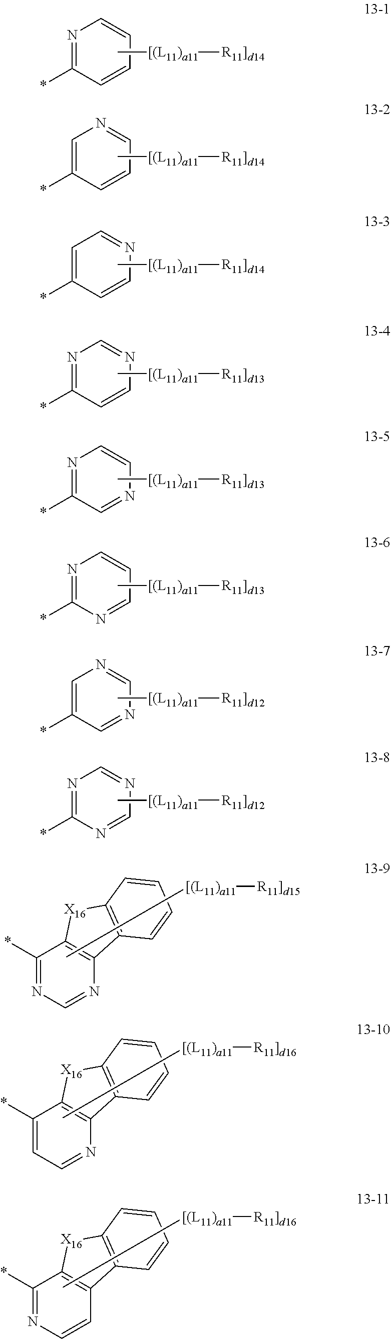

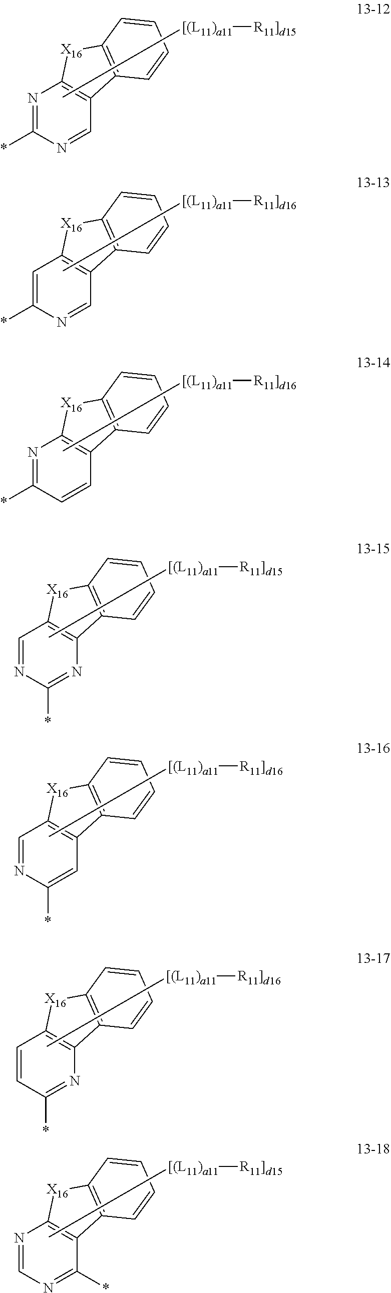

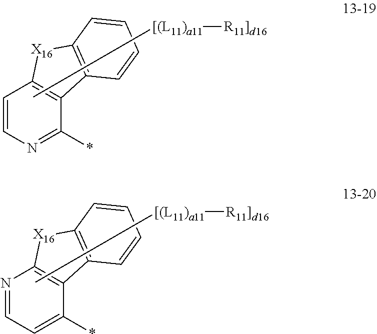

In Formulae 13-1 to 13-20,

X₁₆ may be N-[(L₁₂)ₐ₁₂-R₁₂], C(R₁₄)(R₁₅), O, or S,

L₁₁ and L₁₂ may each independently have the same definition as that of L₃,

a11 and a12 may each independently have the same definition as that of c3,

R₁₁, R₁₂, R₁₄, and R₁₅ may each independently have the same definition as that of R₁,

d16 may be an integer from 0 to 6,

d15 may be an integer from 0 to 5,

d14 may be an integer from 0 to 4,

d13 may be an integer from 0 to 3,

d12 may be an integer from 0 to 2, and

* indicates a binding site to a neighboring atom.

In one embodiment, in Formula 11, R₁, R₂, R₅, and R₆ may each independently be a hydrogen, a deuterium, a C₁-C₁₀ alkyl group, a C₁-C₁₀ alkoxy group, a phenyl group, a biphenyl group, a terphenyl group, a naphthyl group, a fluorenyl group, a pyridinyl group, a pyrimidinyl group, a pyrazinyl group, a pyridazinyl group, a triazinyl group, a carbazolyl group, a dibenzofuranyl group, a dibenzothiophenyl group, an indolocarbazolyl group, —Si(Q₁)(Q₂)(Q₃), or —N(Q₄)(Q₅), and Q₁ to Q₅ may each independently be a C₁-C₁₀ alkyl group, a C₁-C₁₀ alkoxy group, a phenyl group, a biphenyl group, a terphenyl group, or a naphthyl group.

In Formula 11, a1 and a2 each indicate the number of R₁ and the number of R₂, and may each independently be an integer from 0 to 10. When a1 is two or more, two or more R₁ may be identical to or different from each other, and when a2 is two or more, two or more R₂ may be identical to or different from each other.

In one embodiment, the thermally activated delayed fluorescence emitter may include one of compounds represented by Formulae 11-1 to 11-7, or any combination thereof, but embodiments of the present disclosure are not limited thereto:

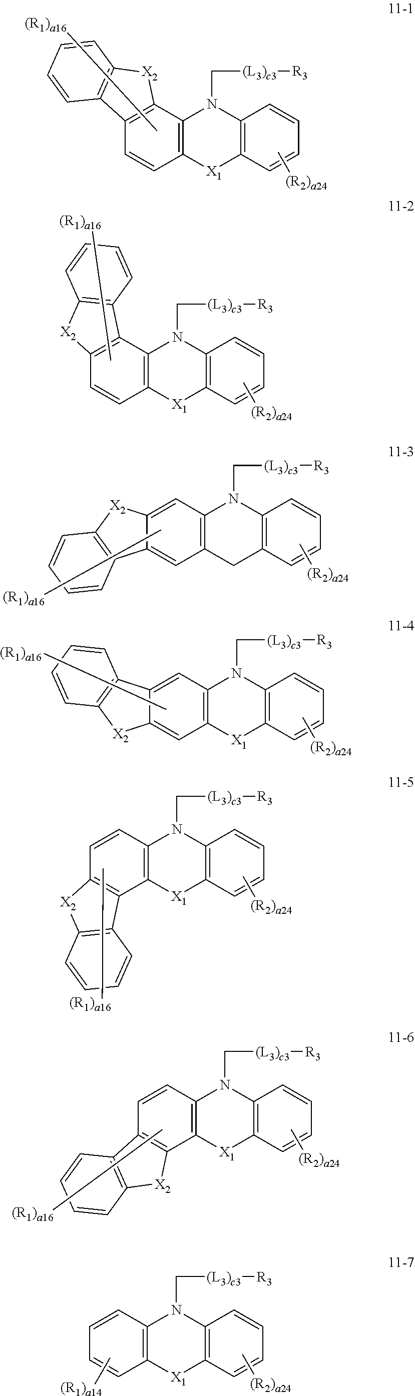

In Formulae 11-1 to 11-7, X₁, L₃, c3, and R₁ to R₃ may each independently be the same as described above, X₂ may be N-[(L₅)_{c5}-R₇], C(R₈)(R₉), O, or S,

L₅ and c5 may each independently have the same definition as that of L₃ and c3,

R₇ may have the same definition as that of R₃,

R₈ and R₉ may each independently have the same definition as that of Rₛ and Re,

a16 may be an integer from 0 to 6, and

a14 and a24 may each independently be an integer from 0 to 4.

In one embodiment, in Formulae 11-1 to 11-17, 1) when X₂ is C(R₈)(R₉), O, or S, R₃ may include a Tπ electron-depleted nitrogen-containing cyclic group, or 2) when X₂ is N-[(L₅)_{c5}-R₇], R₃, R₇, or a combination thereof may include a π electron-depleted nitrogen-containing cyclic group.

In one or more embodiments, in Formulae 11-1 to 11-17, 1) when X₂ is C(R₈)(R₉), O, or S, R₃, or 2) when X₂ is N-[(L₅)_{c5}-R₇], R₃ and R₇, may each independently be:

a group represented by Formula 13(1) or 13(2) (for example, a group represented by one of Formulae 13-1 to 13-20);

a phenyl group, a biphenyl group, a terphenyl group, a naphthyl group, a fluorenyl group, a quinolinyl group, an isoquinolinyl group, a carbazolyl group, a dibenzofuranyl group, a dibenzothiophenyl group, or an indolocarbazolyl group; or

a phenyl group, a biphenyl group, a terphenyl group, a naphthyl group, a fluorenyl group, a quinolinyl group, an isoquinolinyl group, a carbazolyl group, a dibenzofuranyl group, a dibenzothiophenyl group, and an indolocarbazolyl group, each substituted with a deuterium, a C₁-C₁₀ alkyl group, a C₁-C₁₀ alkoxy group, a phenyl group, a biphenyl group, a terphenyl group, a naphthyl group, a fluorenyl group, a pyridinyl group, a pyrimidinyl group, a pyrazinyl group, a pyridazinyl group, a triazinyl group, a quinolinyl group, an isoquinolinyl group, a carbazolyl group, a dibenzofuranyl group, a dibenzothiophenyl group, an indolocarbazolyl group, —Si(Q₃₁)(Q₃₂)(Q₃₃), or a combination thereof,

wherein 1) when X₂ is C(R₈)(R₉), O, or S, R₃, or 2) when X₂ is N-[(L₅)_{c5}-R₇], R₃, R₇, or a combination thereof may be a group represented by Formula 13(1) or 13(2) (for example, a group represented by one of Formulae 13-1 to 13-20).

In one embodiment, the thermally activated delayed fluorescence emitter may include a compound represented by Formula 14A:

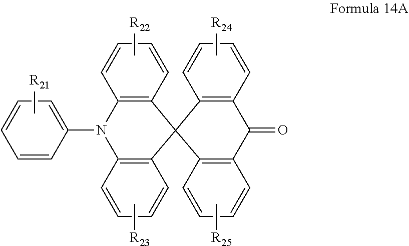

In Formula 14A, R₂₁ to R₂₅ may each independently be a hydrogen, a deuterium, a cyano group, a C₁-C₁₀ alkyl group, a phenyl group, a biphenyl group, or a terphenyl group.

In one embodiment, the thermally activated delayed fluorescence emitter may not include a cyano group.

The thermally activated delayed fluorescence emitter may include one of Compounds D1-1 to D1-83, one of D2-1 to D2-81, one of D3-1 to D3-81, one of D201 to D211, one of Compounds 1 to 1030_{[u1]}, or a combination thereof, but embodiments of the present disclosure are not limited thereto:

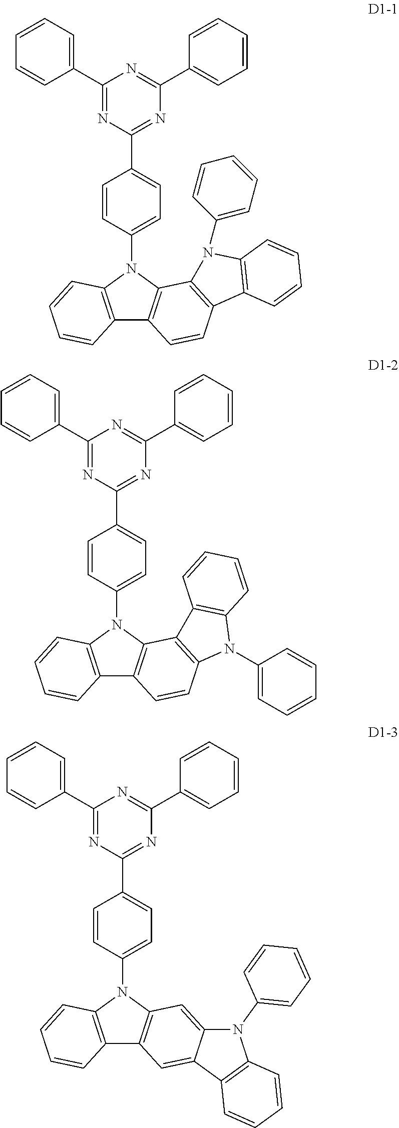

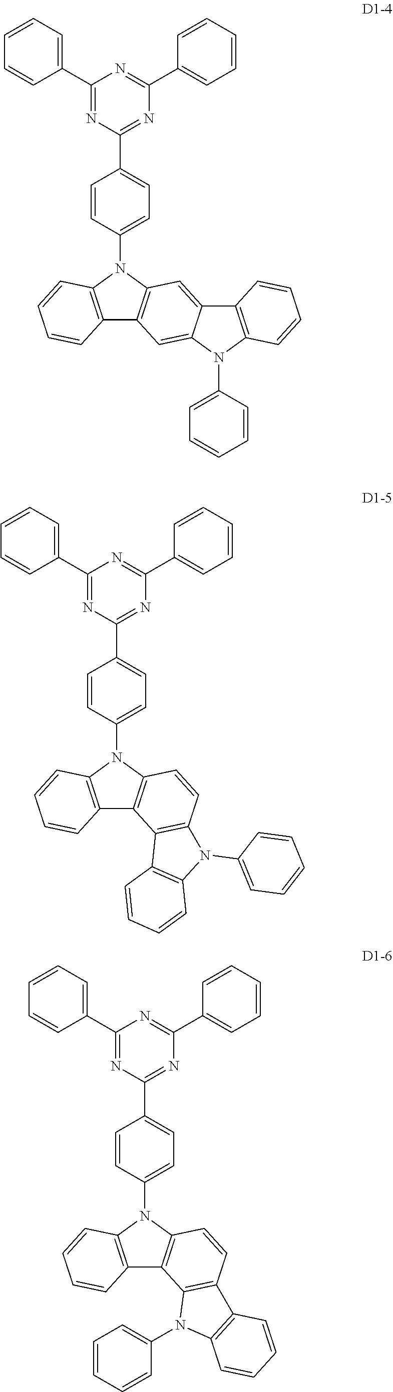 [SMILES: c1ccc(-c2nc(-c3ccccc3)nc(-c3ccc(-n4c5ccccc5c5cc6c(cc54)c4ccccc4n6-c4ccccc4)cc3)n2)cc1 ; c1ccc(-c2nc(-c3ccccc3)nc(-c3ccc(-n4c5ccccc5c5c6c7ccccc7n(-c7ccccc7)c6ccc54)cc3)n2)cc1 ; c1ccc(-c2nc(-c3ccccc3)nc(-c3ccc(-n4c5ccccc5c5c4ccc4c6ccccc6n(-c6ccccc6)c45)cc3)n2)cc1]

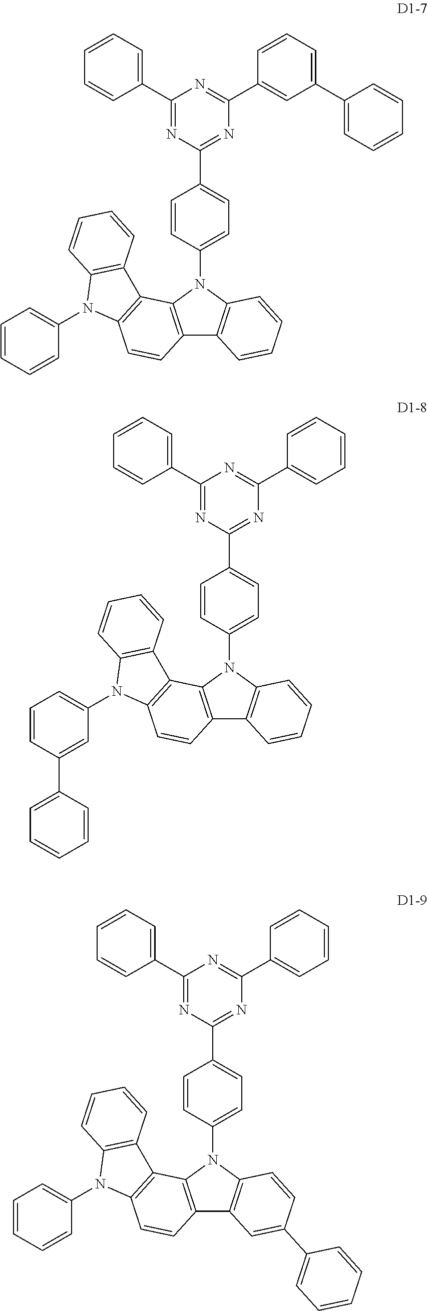

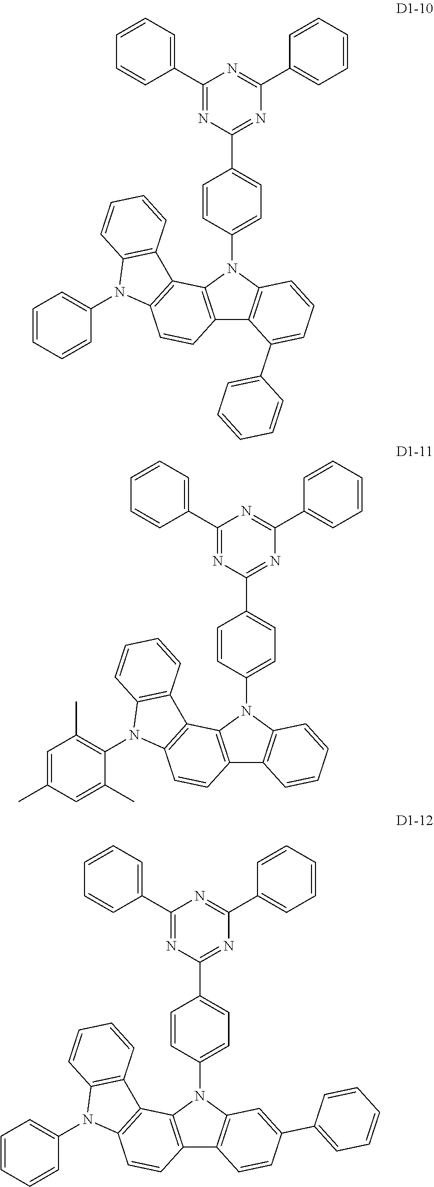 [SMILES: c1ccc(-c2nc(-c3ccccc3)nc(-c3ccc(-n4c5cccc(-c6ccccc6)c5c5ccc6c(c7ccccc7n6-c6ccccc6)c54)cc3)n2)cc1 ; Cc1cc(C)c(-n2c3ccccc3c3c2ccc2c4ccccc4n(-c4ccc(-c5nc(-c6ccccc6)nc(-c6ccccc6)n5)cc4)c23)c(C)c1 ; c1ccc(-c2ccc3c4ccc5c(c6ccccc6n5-c5ccccc5)c4n(-c4ccc(-c5nc(-c6ccccc6)nc(-c6ccccc6)n5)cc4)c3c2)cc1]

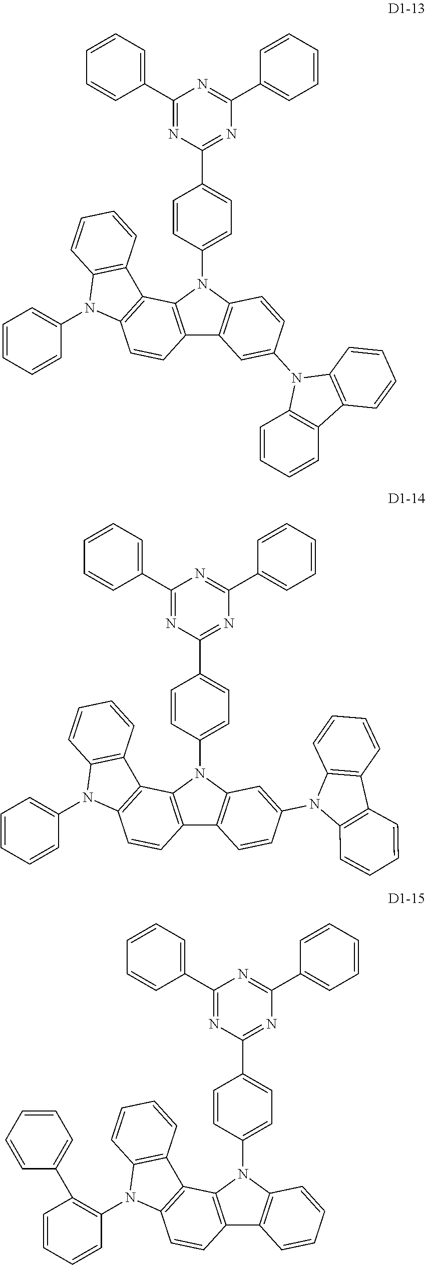

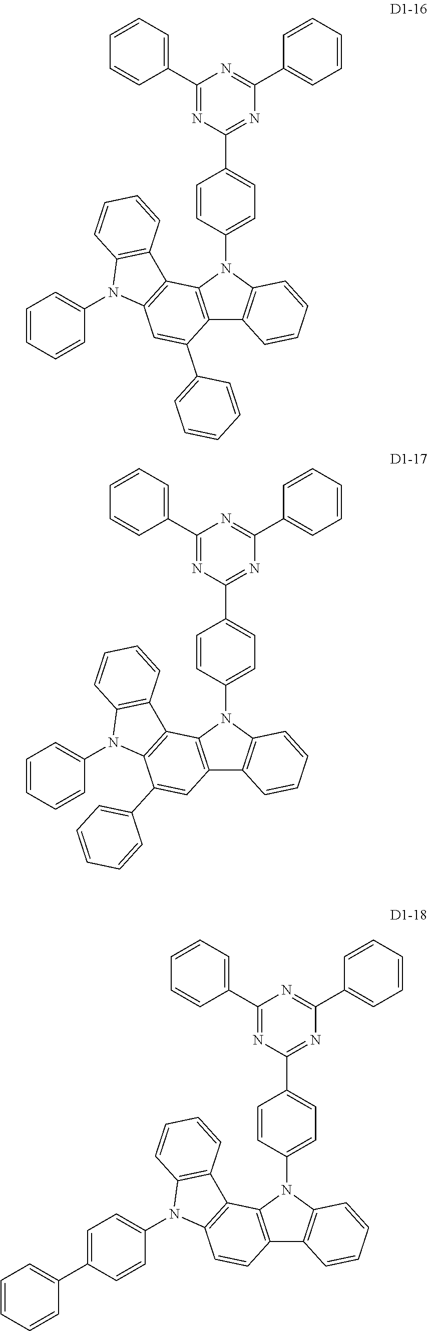 [SMILES: c1ccc(-c2nc(-c3ccccc3)nc(-c3ccc(-n4c5ccccc5c5c(-c6ccccc6)cc6c(c7ccccc7n6-c6ccccc6)c54)cc3)n2)cc1 ; c1ccc(-c2nc(-c3ccccc3)nc(-c3ccc(-n4c5ccccc5c5cc(-c6ccccc6)c6c(c7ccccc7n6-c6ccccc6)c54)cc3)n2)cc1 ; c1ccc(-c2ccc(-n3c4ccccc4c4c3ccc3c5ccccc5n(-c5ccc(-c6nc(-c7ccccc7)nc(-c7ccccc7)n6)cc5)c34)cc2)cc1]

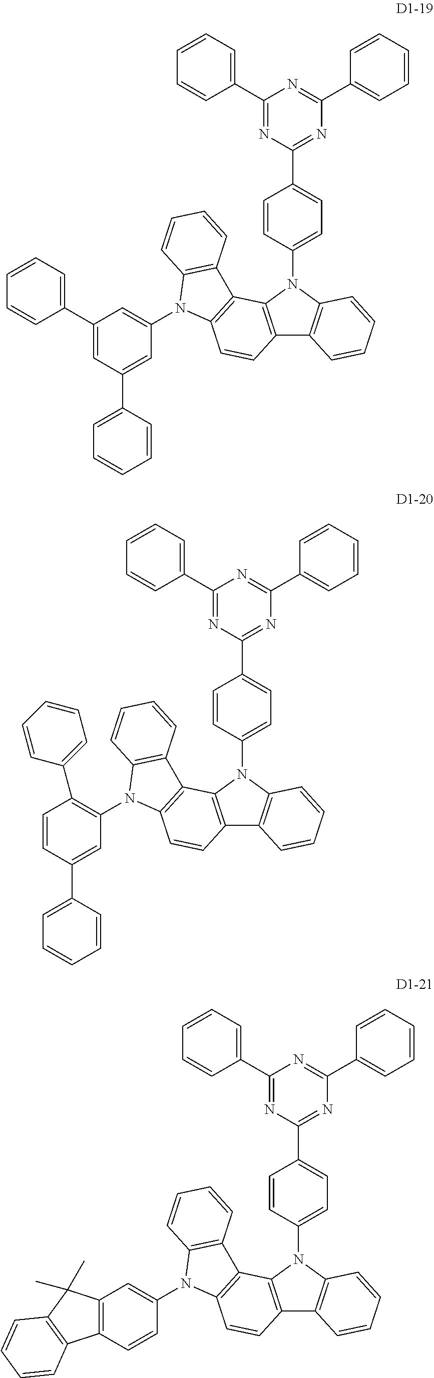 [SMILES: c1ccc(-c2cc(-c3ccccc3)cc(-n3c4ccccc4c4c3ccc3c5ccccc5n(-c5ccc(-c6nc(-c7ccccc7)nc(-c7ccccc7)n6)cc5)c34)c2)cc1 ; c1ccc(-c2ccc(-c3ccccc3)c(-n3c4ccccc4c4c3ccc3c5ccccc5n(-c5ccc(-c6nc(-c7ccccc7)nc(-c7ccccc7)n6)cc5)c34)c2)cc1 ; CC1(C)c2ccccc2-c2ccc(-n3c4ccccc4c4c3ccc3c5ccccc5n(-c5ccc(-c6nc(-c7ccccc7)nc(-c7ccccc7)n6)cc5)c34)cc21]

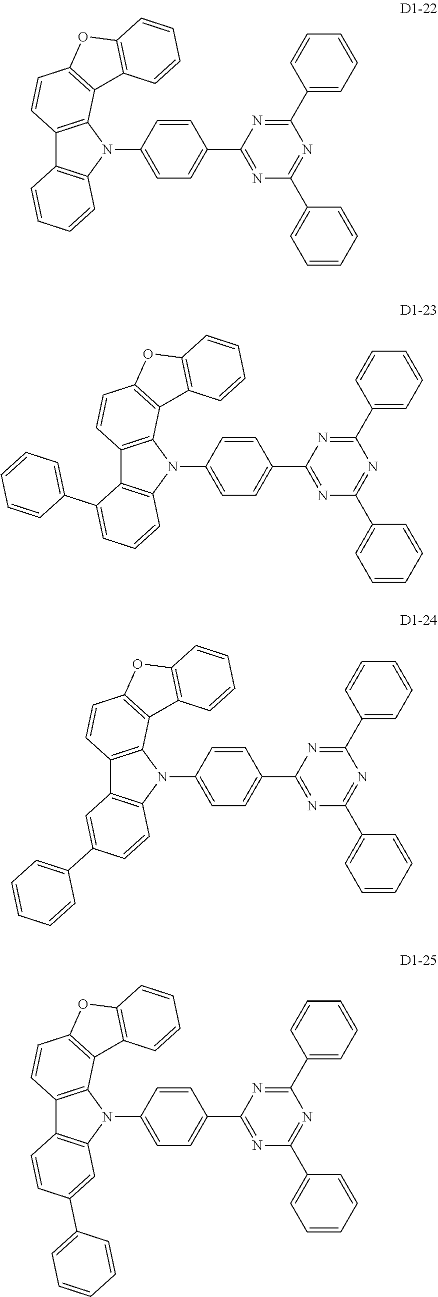 [SMILES: c1ccc(-c2nc(-c3ccccc3)nc(-c3ccc(-n4c5ccccc5c5ccc6oc7ccccc7c6c54)cc3)n2)cc1 ; c1ccc(-c2nc(-c3ccccc3)nc(-c3ccc(-n4c5cccc(-c6ccccc6)c5c5ccc6oc7ccccc7c6c54)cc3)n2)cc1 ; c1ccc(-c2ccc3c(c2)c2ccc4oc5ccccc5c4c2n3-c2ccc(-c3nc(-c4ccccc4)nc(-c4ccccc4)n3)cc2)cc1 ; c1ccc(-c2ccc3c4ccc5oc6ccccc6c5c4n(-c4ccc(-c5nc(-c6ccccc6)nc(-c6ccccc6)n5)cc4)c3c2)cc1]

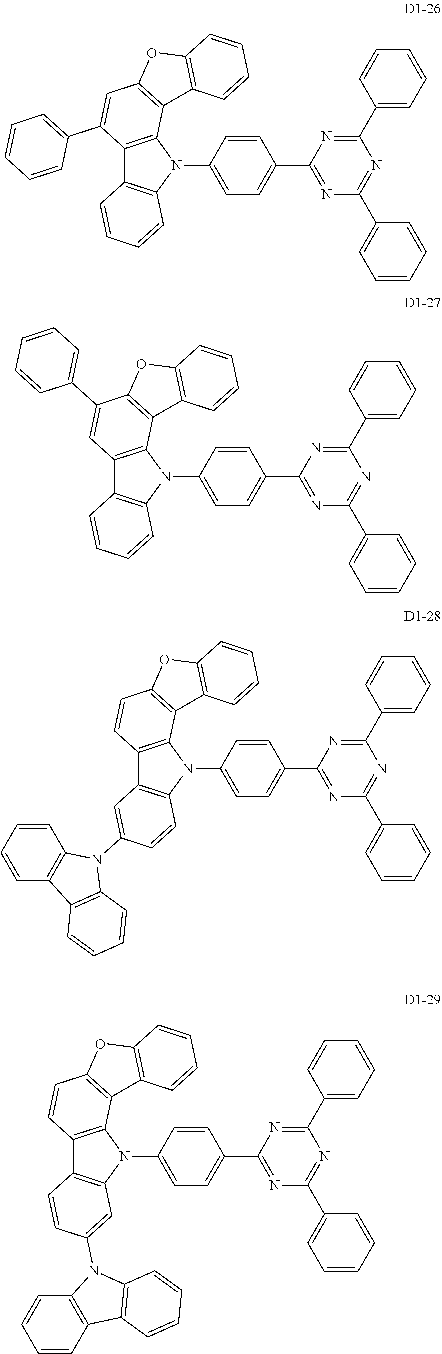 [SMILES: c1ccc(-c2nc(-c3ccccc3)nc(-c3ccc(-n4c5ccccc5c5c(-c6ccccc6)cc6oc7ccccc7c6c54)cc3)n2)cc1 ; c1ccc(-c2nc(-c3ccccc3)nc(-c3ccc(-n4c5ccccc5c5cc(-c6ccccc6)c6oc7ccccc7c6c54)cc3)n2)cc1 ; c1ccc(-c2nc(-c3ccccc3)nc(-c3ccc(-n4c5ccc(-n6c7ccccc7c7ccccc76)cc5c5ccc6oc7ccccc7c6c54)cc3)n2)cc1 ; c1ccc(-c2nc(-c3ccccc3)nc(-c3ccc(-n4c5cc(-n6c7ccccc7c7ccccc76)ccc5c5ccc6oc7ccccc7c6c54)cc3)n2)cc1]

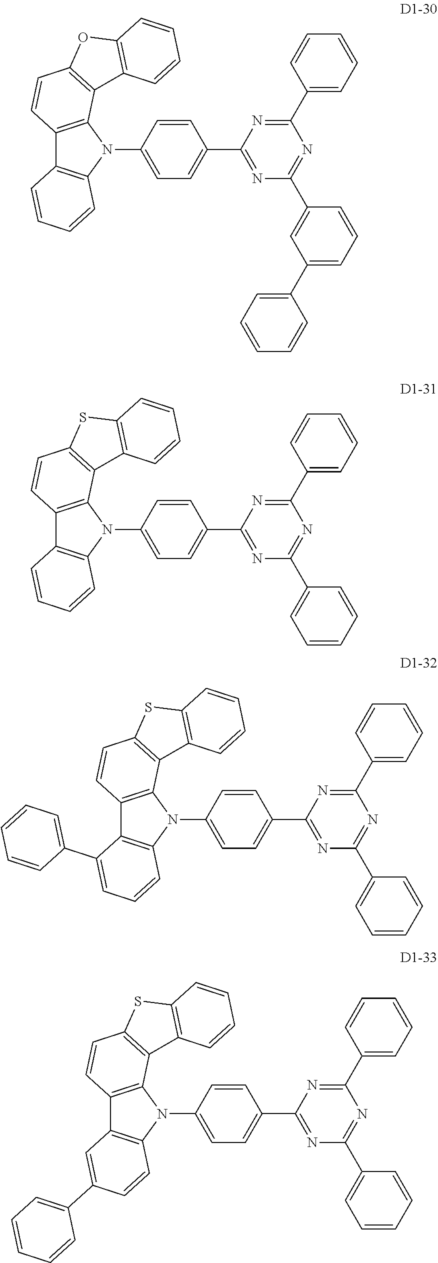 [SMILES: c1ccc(-c2cccc(-c3nc(-c4ccccc4)nc(-c4ccc(-n5c6ccccc6c6ccc7oc8ccccc8c7c65)cc4)n3)c2)cc1 ; c1ccc(-c2nc(-c3ccccc3)nc(-c3ccc(-n4c5ccccc5c5ccc6sc7ccccc7c6c54)cc3)n2)cc1 ; c1ccc(-c2nc(-c3ccccc3)nc(-c3ccc(-n4c5cccc(-c6ccccc6)c5c5ccc6sc7ccccc7c6c54)cc3)n2)cc1 ; c1ccc(-c2ccc3c(c2)c2ccc4sc5ccccc5c4c2n3-c2ccc(-c3nc(-c4ccccc4)nc(-c4ccccc4)n3)cc2)cc1]

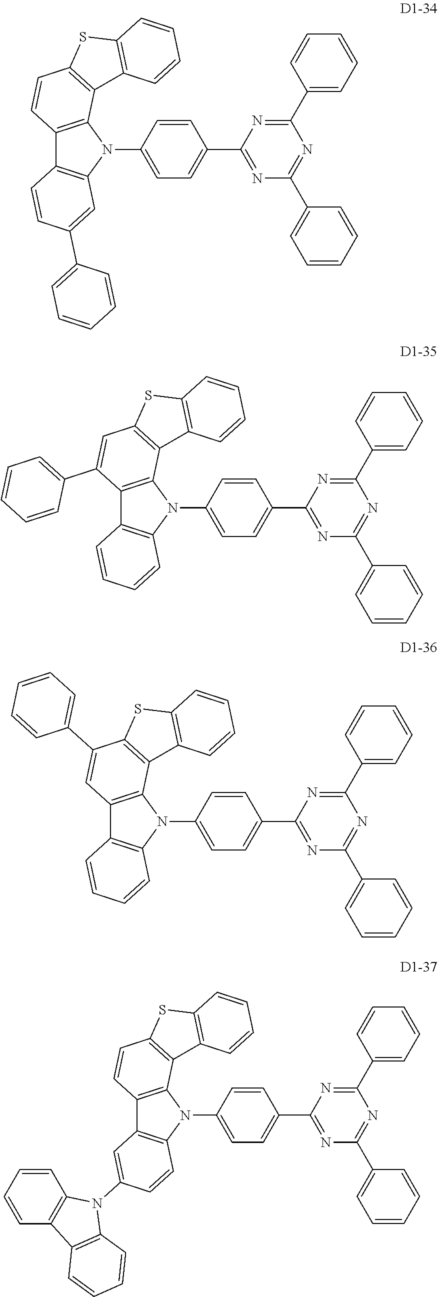 [SMILES: c1ccc(-c2ccc3c4ccc5sc6ccccc6c5c4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccccc5c5c(-c6ccccc6)cc6sc7ccccc7c6c54)cc3)n2)cc1 ; c1ccc(-c2nc(-c3ccccc3)nc(-c3ccc(-n4c5ccccc5c5cc(-c6ccccc6)c6sc7ccccc7c6c54)cc3)n2)cc1 ; c1ccc(-c2nc(-c3ccccc3)nc(-c3ccc(-n4c5ccc(-n6c7ccccc7c7ccccc76)cc5c5ccc6sc7ccccc7c6c54)cc3)n2)cc1]

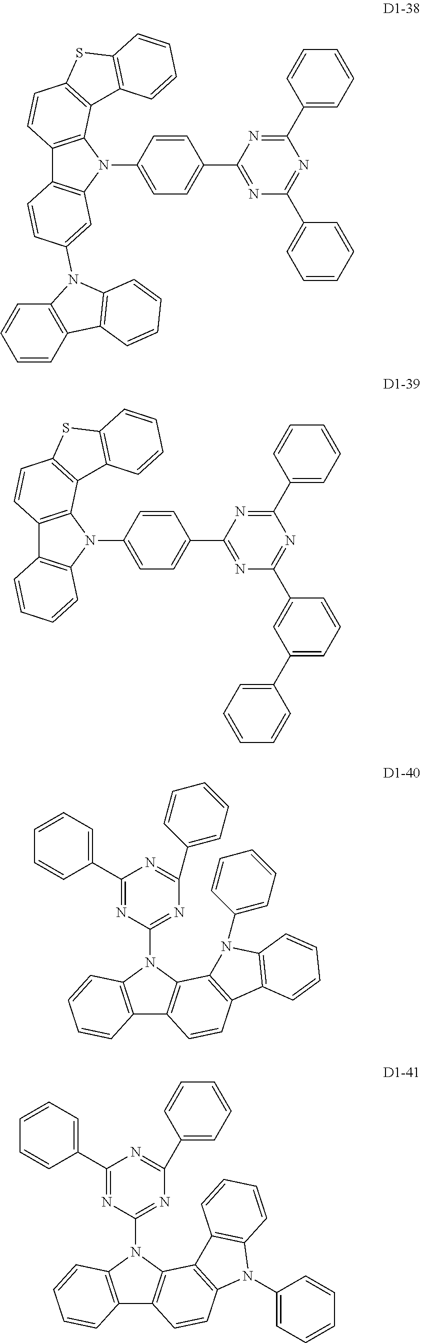

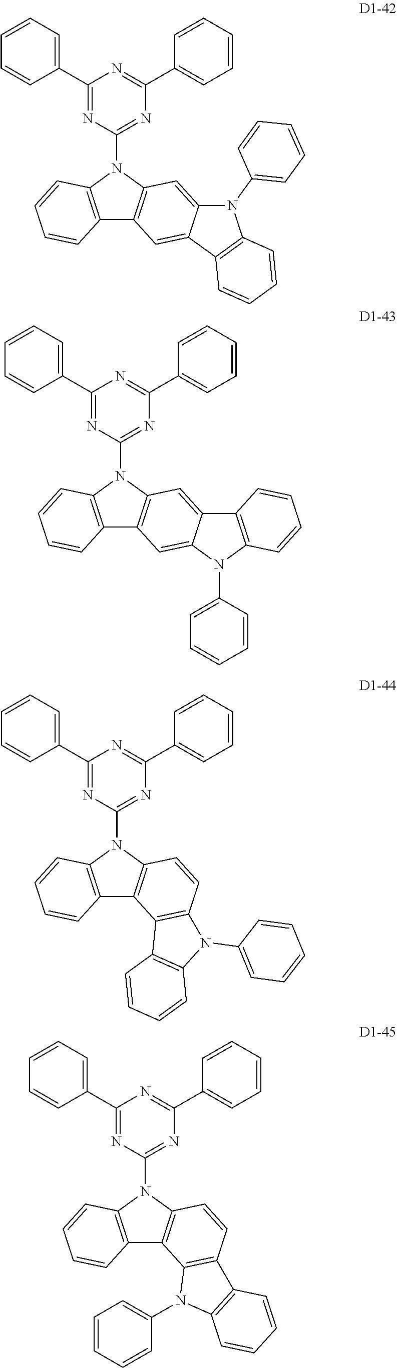

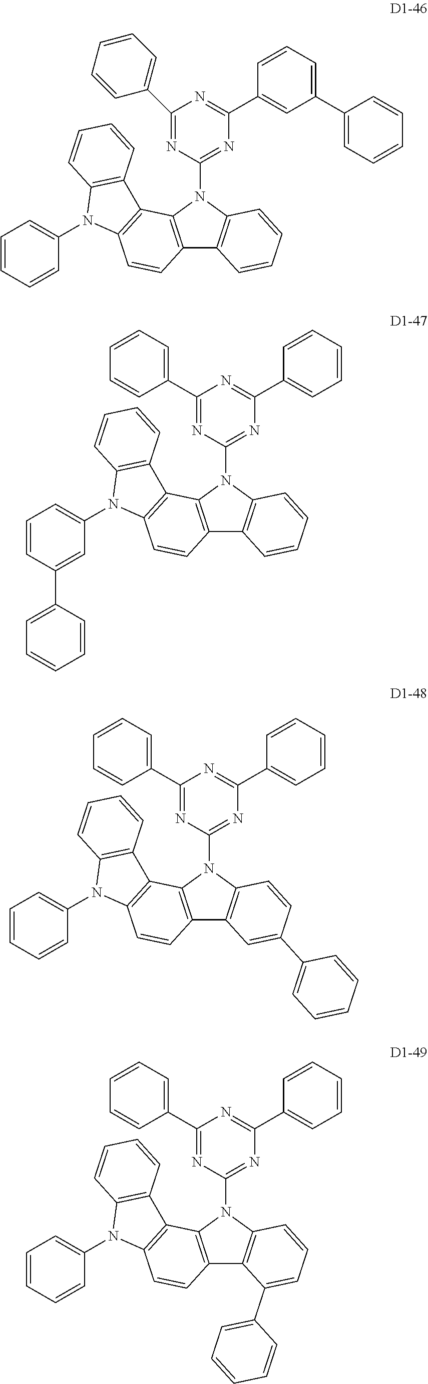

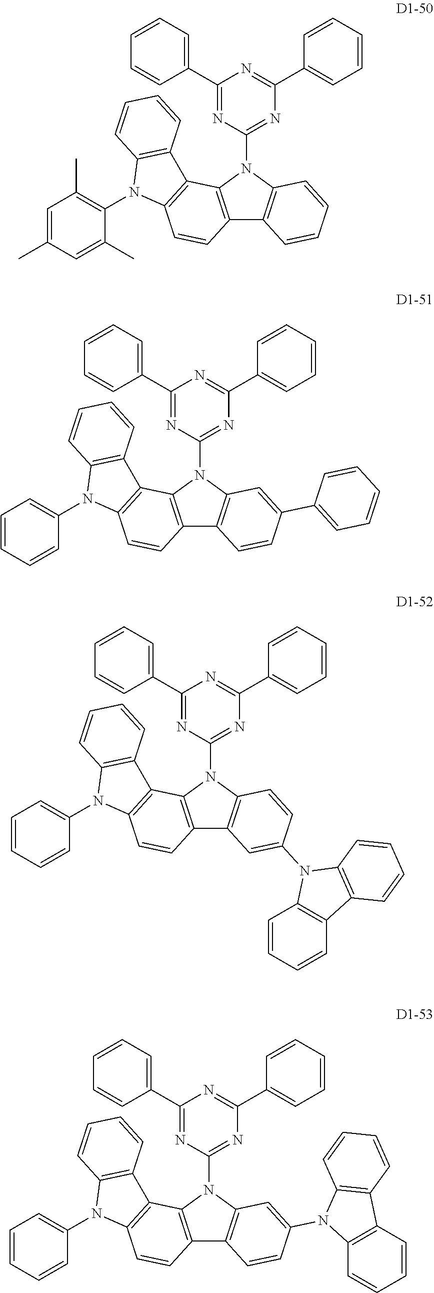 [SMILES: Cc1cc(C)c(-n2c3ccccc3c3c2ccc2c4ccccc4n(-c4nc(-c5ccccc5)nc(-c5ccccc5)n4)c23)c(C)c1 ; c1ccc(-c2ccc3c4ccc5c(c6ccccc6n5-c5ccccc5)c4n(-c4nc(-c5ccccc5)nc(-c5ccccc5)n4)c3c2)cc1 ; c1ccc(-c2nc(-c3ccccc3)nc(-n3c4ccc(-n5c6ccccc6c6ccccc65)cc4c4ccc5c(c6ccccc6n5-c5ccccc5)c43)n2)cc1 ; c1ccc(-c2nc(-c3ccccc3)nc(-n3c4cc(-n5c6ccccc6c6ccccc65)ccc4c4ccc5c(c6ccccc6n5-c5ccccc5)c43)n2)cc1]

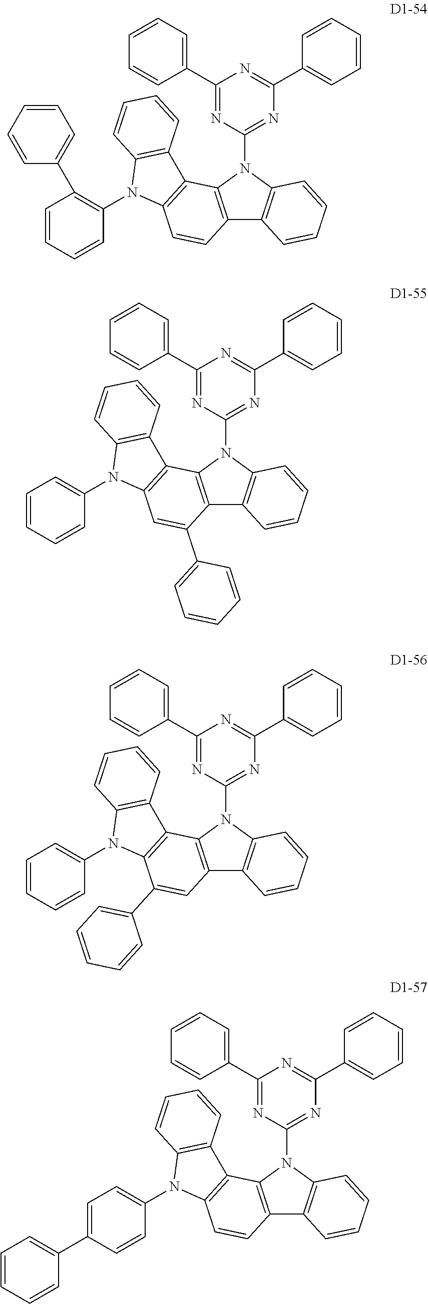 [SMILES: c1ccc(-c2nc(-c3ccccc3)nc(-n3c4ccccc4c4ccc5c(c6ccccc6n5-c5ccccc5-c5ccccc5)c43)n2)cc1 ; c1ccc(-c2nc(-c3ccccc3)nc(-n3c4ccccc4c4c(-c5ccccc5)cc5c(c6ccccc6n5-c5ccccc5)c43)n2)cc1 ; c1ccc(-c2nc(-c3ccccc3)nc(-n3c4ccccc4c4cc(-c5ccccc5)c5c(c6ccccc6n5-c5ccccc5)c43)n2)cc1 ; c1ccc(-c2ccc(-n3c4ccccc4c4c3ccc3c5ccccc5n(-c5nc(-c6ccccc6)nc(-c6ccccc6)n5)c34)cc2)cc1]

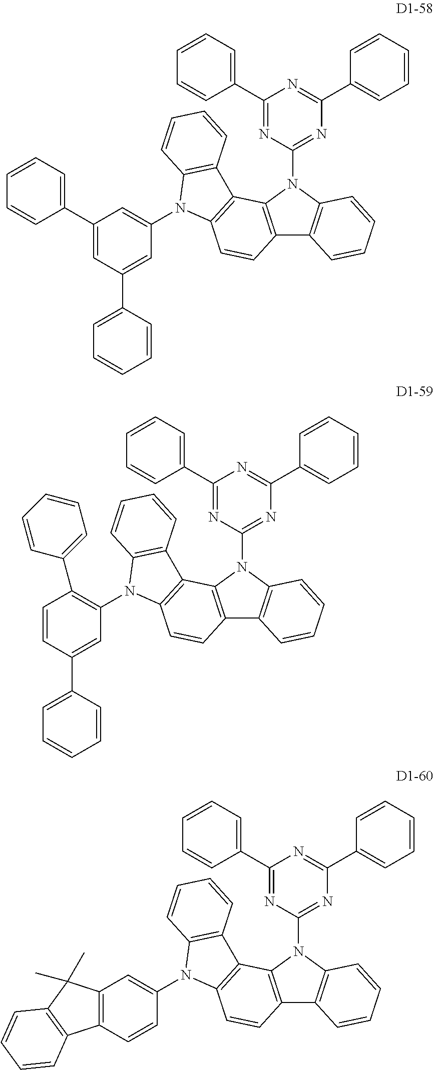 [SMILES: c1ccc(-c2cc(-c3ccccc3)cc(-n3c4ccccc4c4c3ccc3c5ccccc5n(-c5nc(-c6ccccc6)nc(-c6ccccc6)n5)c34)c2)cc1 ; c1ccc(-c2ccc(-c3ccccc3)c(-n3c4ccccc4c4c3ccc3c5ccccc5n(-c5nc(-c6ccccc6)nc(-c6ccccc6)n5)c34)c2)cc1 ; CC1(C)c2ccccc2-c2ccc(-n3c4ccccc4c4c3ccc3c5ccccc5n(-c5nc(-c6ccccc6)nc(-c6ccccc6)n5)c34)cc21]

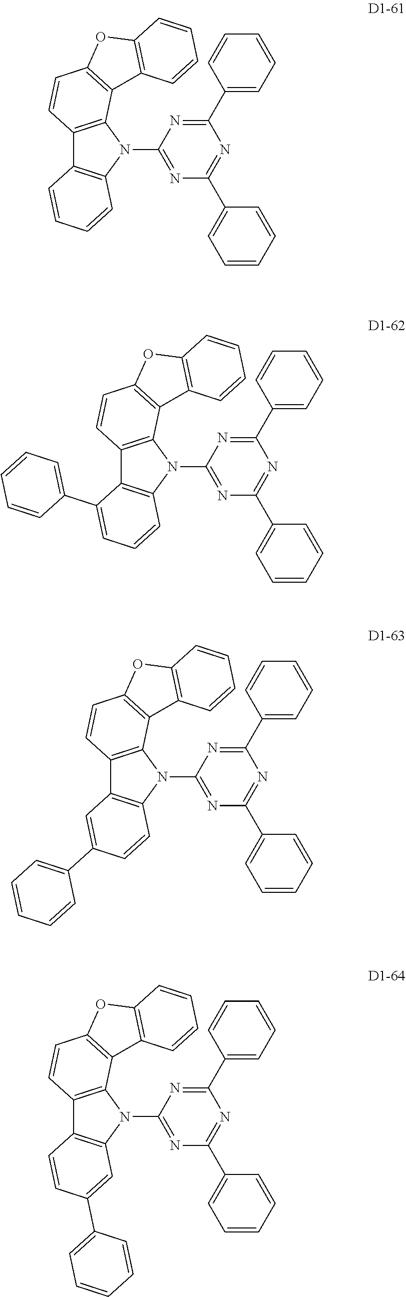 [SMILES: c1ccc(-c2nc(-c3ccccc3)nc(-n3c4ccccc4c4ccc5oc6ccccc6c5c43)n2)cc1 ; c1ccc(-c2nc(-c3ccccc3)nc(-n3c4cccc(-c5ccccc5)c4c4ccc5oc6ccccc6c5c43)n2)cc1 ; c1ccc(-c2ccc3c(c2)c2ccc4oc5ccccc5c4c2n3-c2nc(-c3ccccc3)nc(-c3ccccc3)n2)cc1 ; c1ccc(-c2ccc3c4ccc5oc6ccccc6c5c4n(-c4nc(-c5ccccc5)nc(-c5ccccc5)n4)c3c2)cc1]

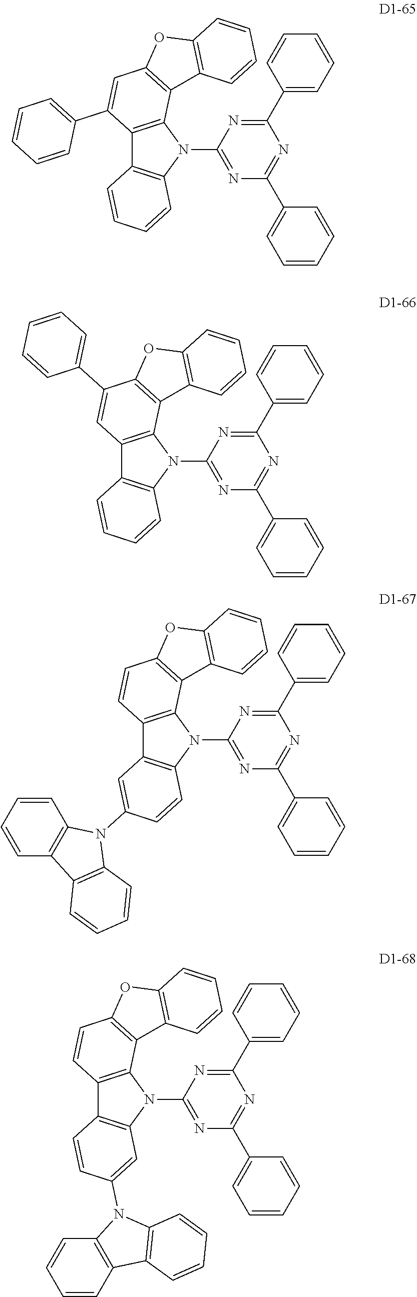

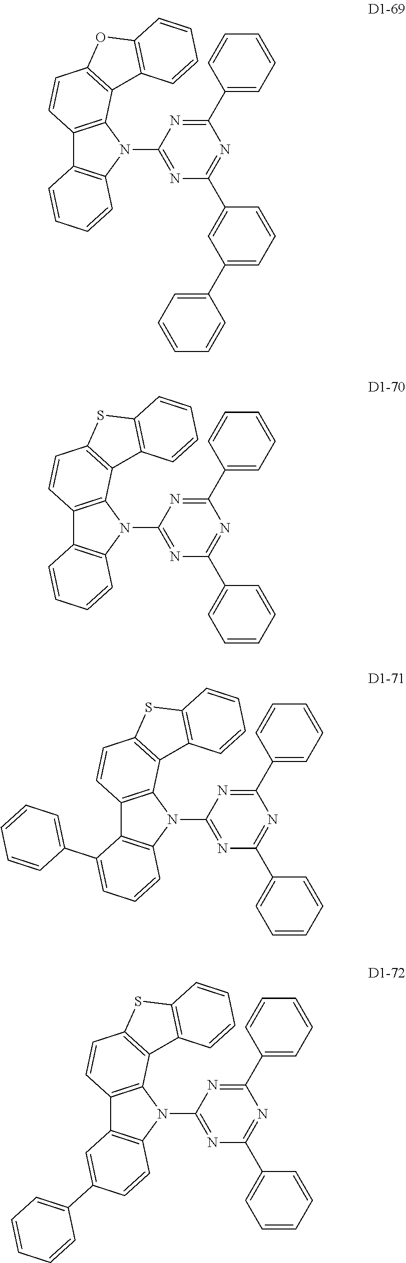 [SMILES: c1ccc(-c2cccc(-c3nc(-c4ccccc4)nc(-n4c5ccccc5c5ccc6oc7ccccc7c6c54)n3)c2)cc1 ; c1ccc(-c2nc(-c3ccccc3)nc(-n3c4ccccc4c4ccc5sc6ccccc6c5c43)n2)cc1 ; c1ccc(-c2nc(-c3ccccc3)nc(-n3c4cccc(-c5ccccc5)c4c4ccc5sc6ccccc6c5c43)n2)cc1 ; c1ccc(-c2ccc3c(c2)c2ccc4sc5ccccc5c4c2n3-c2nc(-c3ccccc3)nc(-c3ccccc3)n2)cc1]

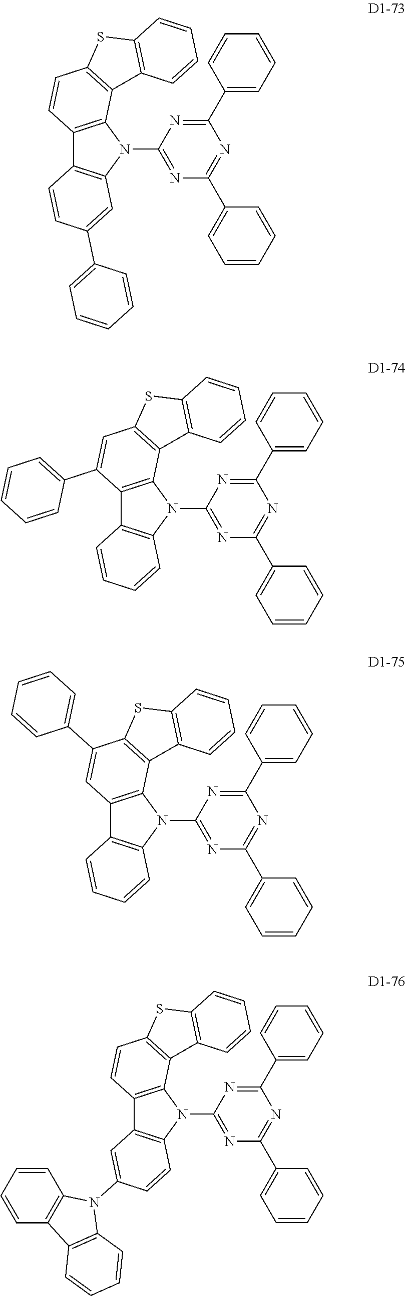 [SMILES: c1ccc(-c2ccc3c4ccc5sc6ccccc6c5c4n(-c4nc(-c5ccccc5)nc(-c5ccccc5)n4)c3c2)cc1 ; c1ccc(-c2nc(-c3ccccc3)nc(-n3c4ccccc4c4c(-c5ccccc5)cc5sc6ccccc6c5c43)n2)cc1 ; c1ccc(-c2nc(-c3ccccc3)nc(-n3c4ccccc4c4cc(-c5ccccc5)c5sc6ccccc6c5c43)n2)cc1 ; c1ccc(-c2nc(-c3ccccc3)nc(-n3c4ccc(-n5c6ccccc6c6ccccc65)cc4c4ccc5sc6ccccc6c5c43)n2)cc1]

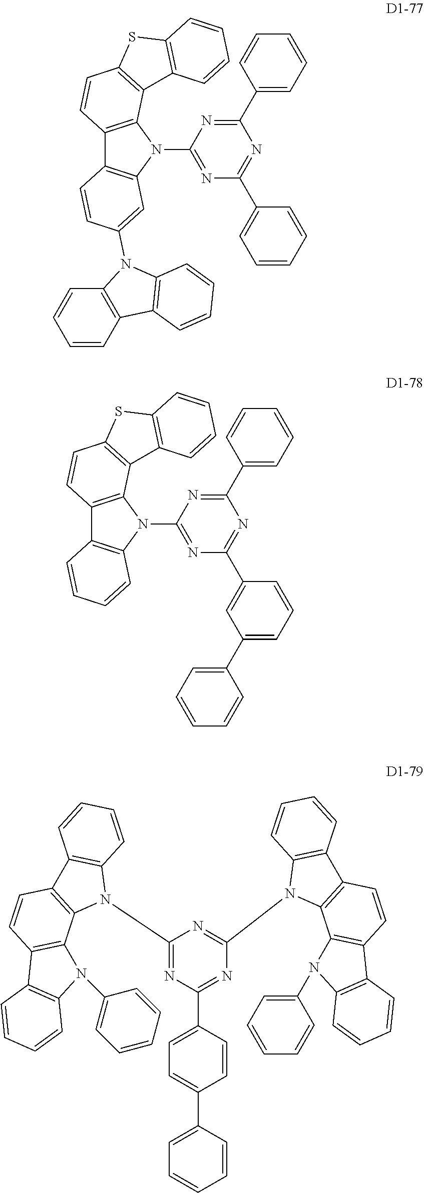 [SMILES: c1ccc(-c2nc(-c3ccccc3)nc(-n3c4cc(-n5c6ccccc6c6ccccc65)ccc4c4ccc5sc6ccccc6c5c43)n2)cc1 ; c1ccc(-c2cccc(-c3nc(-c4ccccc4)nc(-n4c5ccccc5c5ccc6sc7ccccc7c6c54)n3)c2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1]

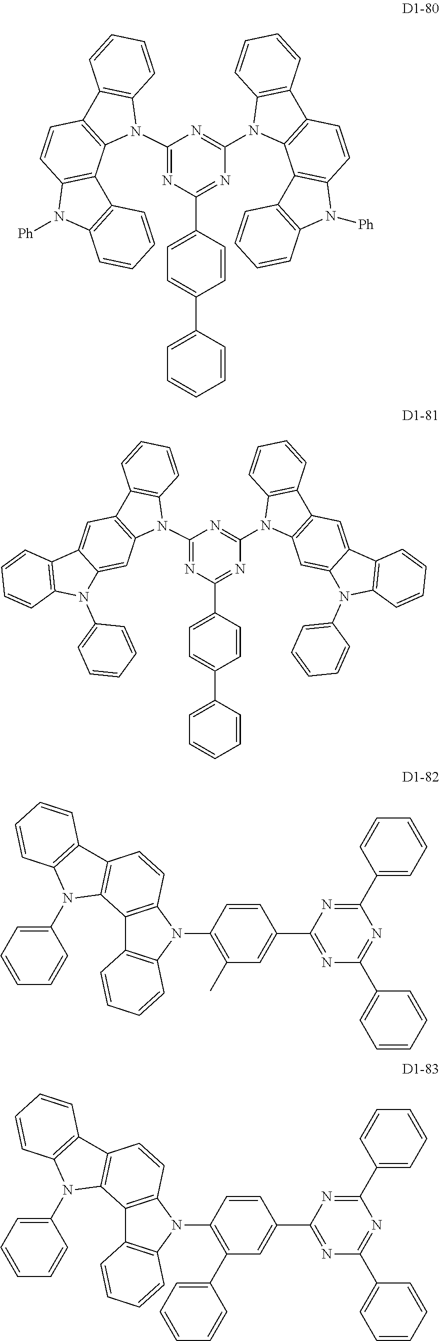 [SMILES: c1ccc(-c2ccc(-c3nc(-n4c5ccccc5c5ccc6c(c7ccccc7n6-c6ccccc6)c54)nc(-n4c5ccccc5c5ccc6c(c7ccccc7n6-c6ccccc6)c54)n3)cc2)cc1 ; c1ccc(-c2ccc(-c3nc(-n4c5ccccc5c5cc6c7ccccc7n(-c7ccccc7)c6cc54)nc(-n4c5ccccc5c5cc6c7ccccc7n(-c7ccccc7)c6cc54)n3)cc2)cc1 ; Cc1cc(-c2nc(-c3ccccc3)nc(-c3ccccc3)n2)ccc1-n1c2ccccc2c2c1ccc1c3ccccc3n(-c3ccccc3)c12 ; c1ccc(-c2nc(-c3ccccc3)nc(-c3ccc(-n4c5ccccc5c5c4ccc4c6ccccc6n(-c6ccccc6)c45)c(-c4ccccc4)c3)n2)cc1]

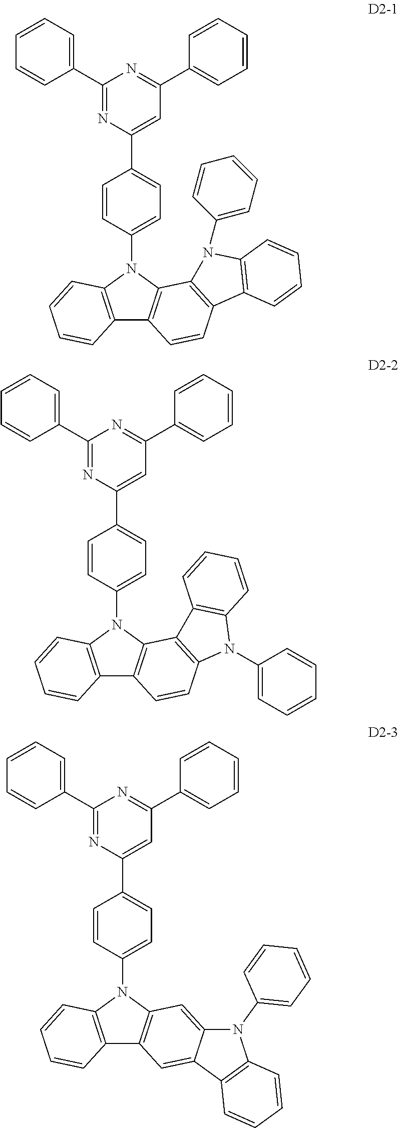

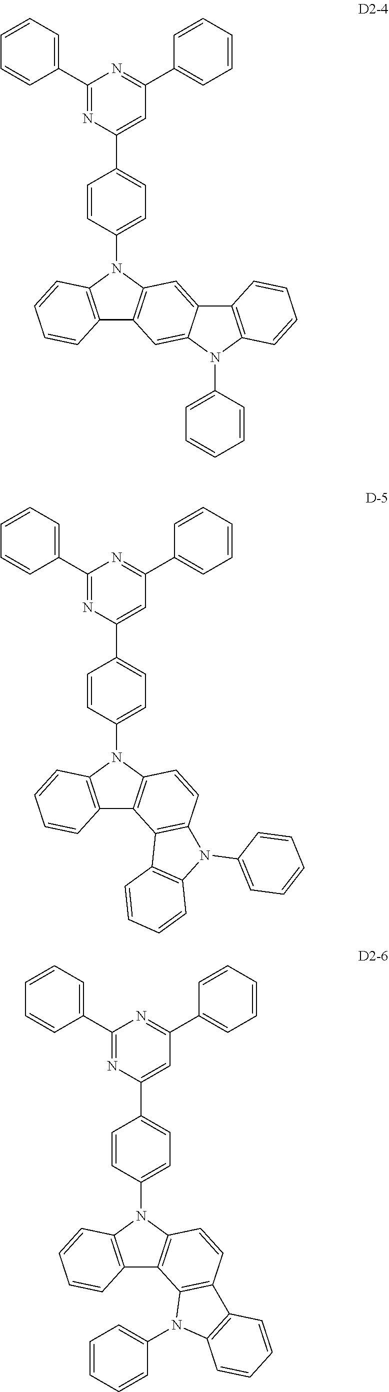 [SMILES: c1ccc(-c2cc(-c3ccc(-n4c5ccccc5c5cc6c(cc54)c4ccccc4n6-c4ccccc4)cc3)nc(-c3ccccc3)n2)cc1 ; c1ccc(-c2cc(-c3ccc(-n4c5ccccc5c5c6c7ccccc7n(-c7ccccc7)c6ccc54)cc3)nc(-c3ccccc3)n2)cc1 ; c1ccc(-c2cc(-c3ccc(-n4c5ccccc5c5c4ccc4c6ccccc6n(-c6ccccc6)c45)cc3)nc(-c3ccccc3)n2)cc1]

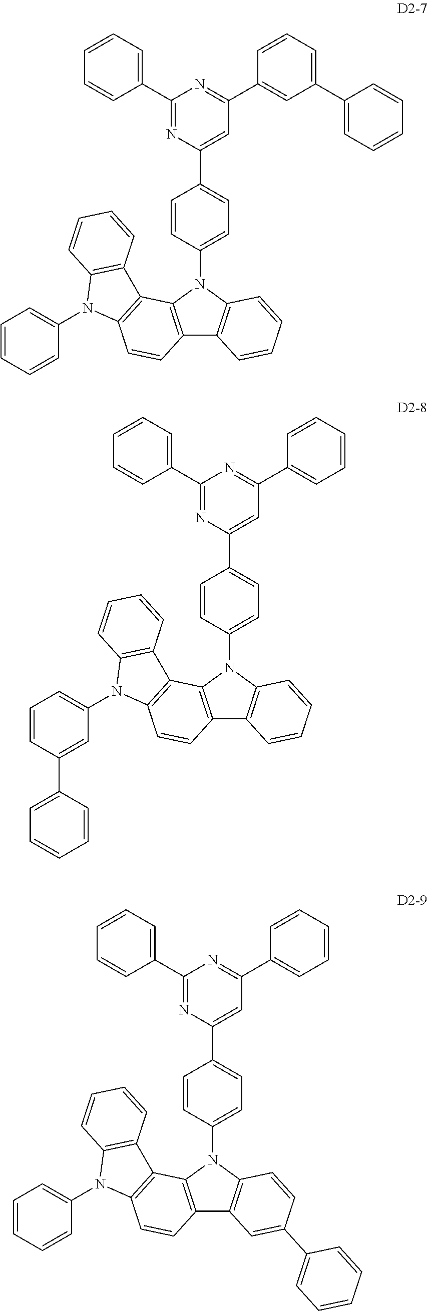

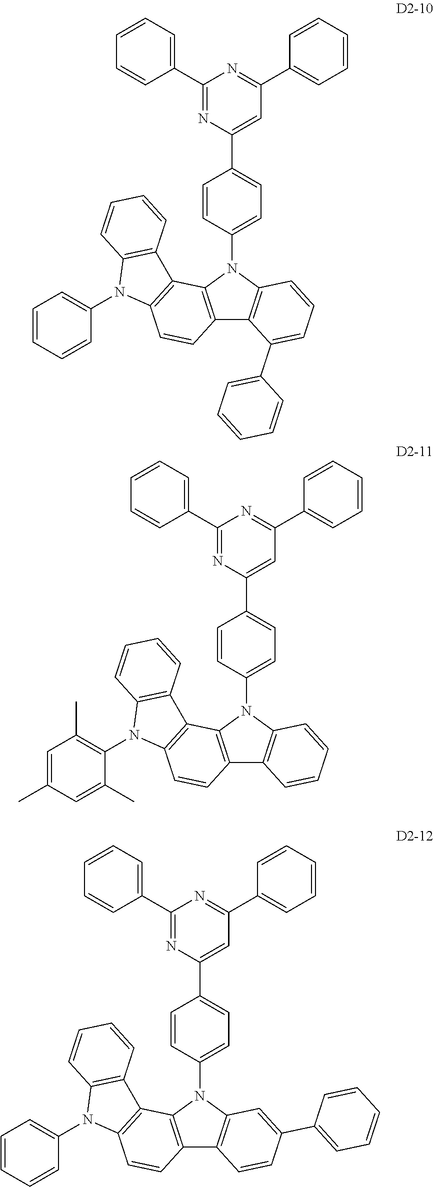 [SMILES: c1ccc(-c2cc(-c3ccc(-n4c5cccc(-c6ccccc6)c5c5ccc6c(c7ccccc7n6-c6ccccc6)c54)cc3)nc(-c3ccccc3)n2)cc1 ; Cc1cc(C)c(-n2c3ccccc3c3c2ccc2c4ccccc4n(-c4ccc(-c5cc(-c6ccccc6)nc(-c6ccccc6)n5)cc4)c23)c(C)c1 ; c1ccc(-c2ccc3c4ccc5c(c6ccccc6n5-c5ccccc5)c4n(-c4ccc(-c5cc(-c6ccccc6)nc(-c6ccccc6)n5)cc4)c3c2)cc1]

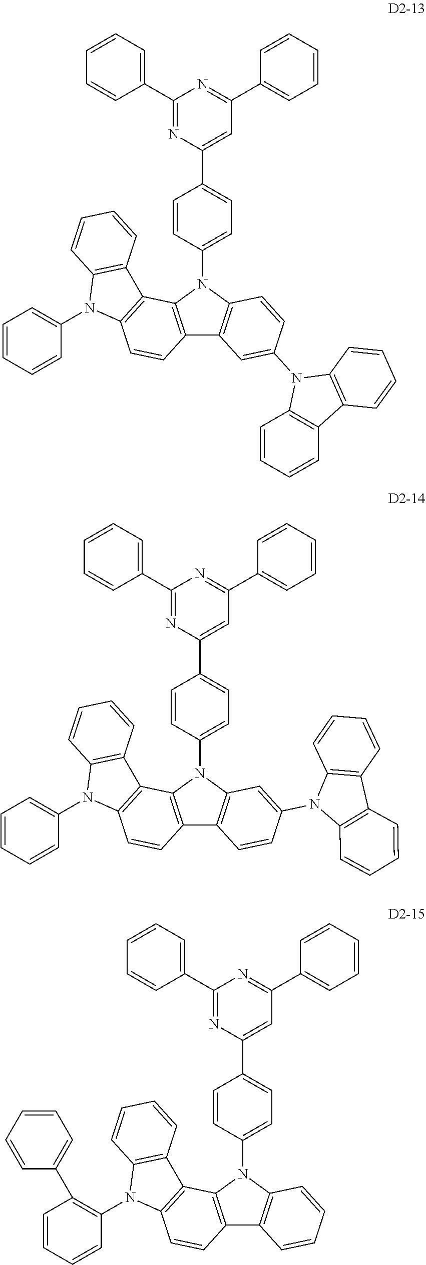

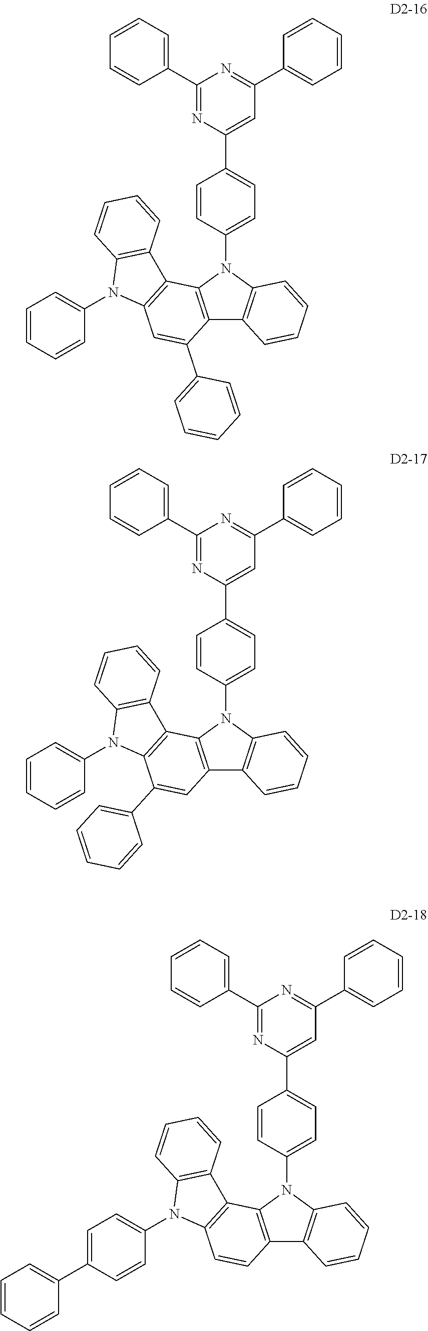 [SMILES: c1ccc(-c2cc(-c3ccc(-n4c5ccccc5c5c(-c6ccccc6)cc6c(c7ccccc7n6-c6ccccc6)c54)cc3)nc(-c3ccccc3)n2)cc1 ; c1ccc(-c2cc(-c3ccc(-n4c5ccccc5c5cc(-c6ccccc6)c6c(c7ccccc7n6-c6ccccc6)c54)cc3)nc(-c3ccccc3)n2)cc1 ; c1ccc(-c2ccc(-n3c4ccccc4c4c3ccc3c5ccccc5n(-c5ccc(-c6cc(-c7ccccc7)nc(-c7ccccc7)n6)cc5)c34)cc2)cc1]

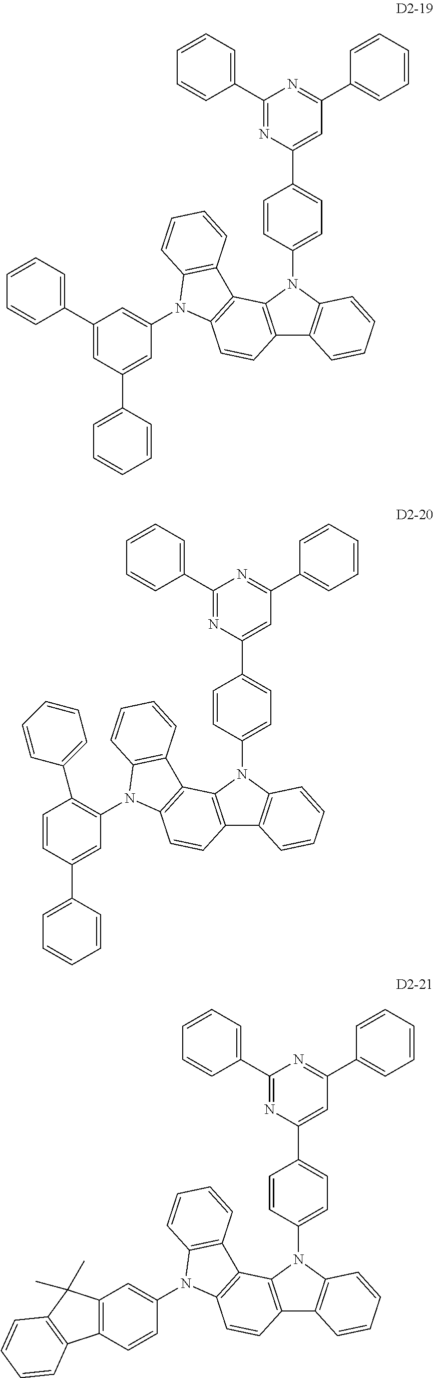 [SMILES: c1ccc(-c2cc(-c3ccccc3)cc(-n3c4ccccc4c4c3ccc3c5ccccc5n(-c5ccc(-c6cc(-c7ccccc7)nc(-c7ccccc7)n6)cc5)c34)c2)cc1 ; c1ccc(-c2ccc(-c3ccccc3)c(-n3c4ccccc4c4c3ccc3c5ccccc5n(-c5ccc(-c6cc(-c7ccccc7)nc(-c7ccccc7)n6)cc5)c34)c2)cc1 ; CC1(C)c2ccccc2-c2ccc(-n3c4ccccc4c4c3ccc3c5ccccc5n(-c5ccc(-c6cc(-c7ccccc7)nc(-c7ccccc7)n6)cc5)c34)cc21]

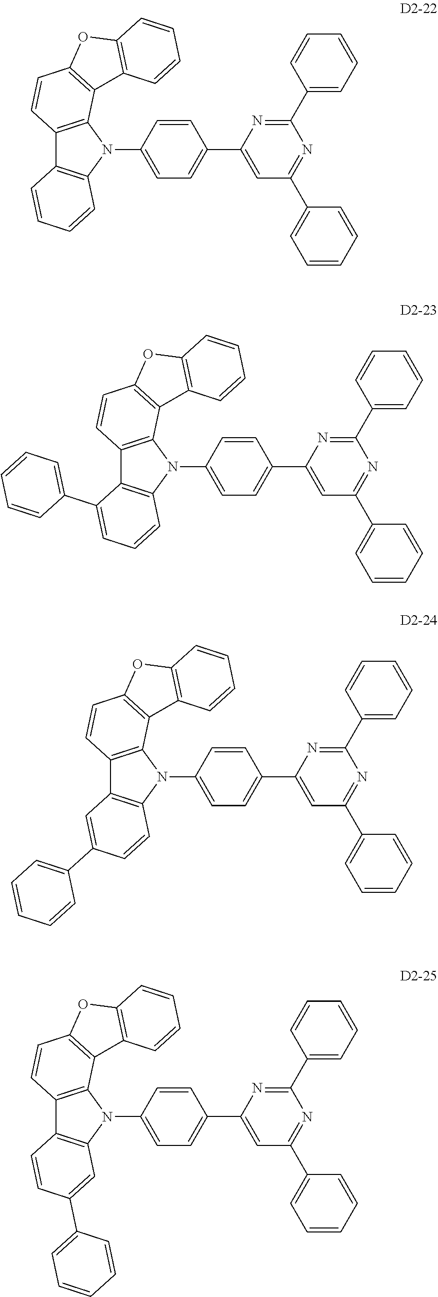 [SMILES: c1ccc(-c2cc(-c3ccc(-n4c5ccccc5c5ccc6oc7ccccc7c6c54)cc3)nc(-c3ccccc3)n2)cc1 ; c1ccc(-c2cc(-c3ccc(-n4c5cccc(-c6ccccc6)c5c5ccc6oc7ccccc7c6c54)cc3)nc(-c3ccccc3)n2)cc1 ; c1ccc(-c2ccc3c(c2)c2ccc4oc5ccccc5c4c2n3-c2ccc(-c3cc(-c4ccccc4)nc(-c4ccccc4)n3)cc2)cc1 ; c1ccc(-c2ccc3c4ccc5oc6ccccc6c5c4n(-c4ccc(-c5cc(-c6ccccc6)nc(-c6ccccc6)n5)cc4)c3c2)cc1]

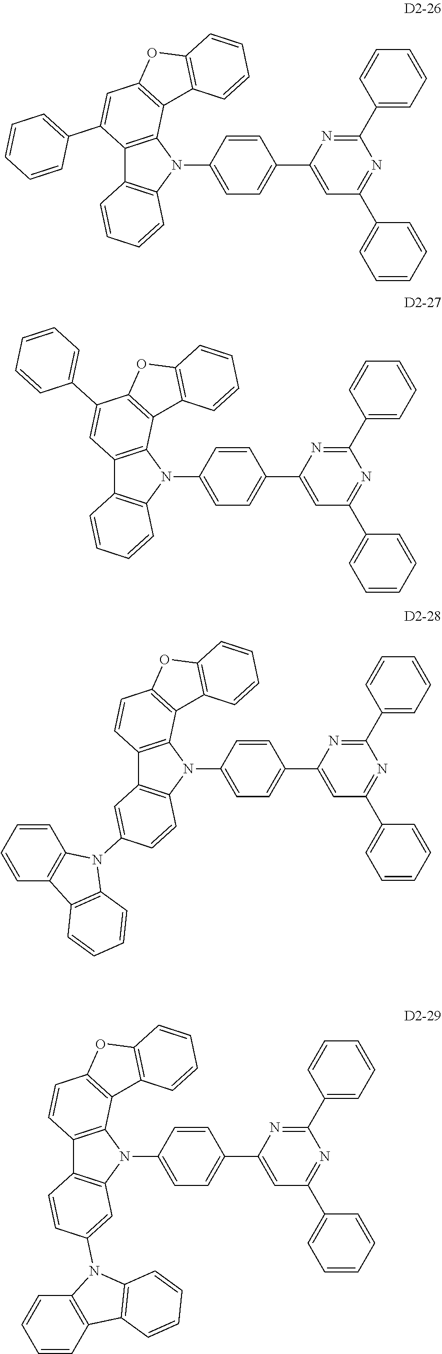 [SMILES: c1ccc(-c2cc(-c3ccc(-n4c5ccccc5c5c(-c6ccccc6)cc6oc7ccccc7c6c54)cc3)nc(-c3ccccc3)n2)cc1 ; c1ccc(-c2cc(-c3ccc(-n4c5ccccc5c5cc(-c6ccccc6)c6oc7ccccc7c6c54)cc3)nc(-c3ccccc3)n2)cc1 ; c1ccc(-c2cc(-c3ccc(-n4c5ccc(-n6c7ccccc7c7ccccc76)cc5c5ccc6oc7ccccc7c6c54)cc3)nc(-c3ccccc3)n2)cc1 ; c1ccc(-c2cc(-c3ccc(-n4c5cc(-n6c7ccccc7c7ccccc76)ccc5c5ccc6oc7ccccc7c6c54)cc3)nc(-c3ccccc3)n2)cc1]

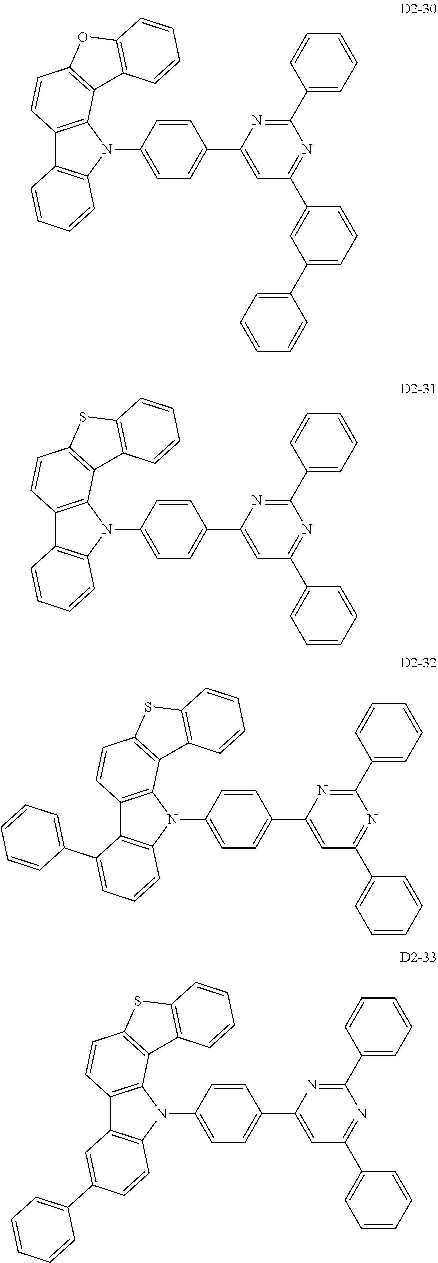 [SMILES: c1ccc(-c2cccc(-c3cc(-c4ccc(-n5c6ccccc6c6ccc7oc8ccccc8c7c65)cc4)nc(-c4ccccc4)n3)c2)cc1 ; c1ccc(-c2cc(-c3ccc(-n4c5ccccc5c5ccc6sc7ccccc7c6c54)cc3)nc(-c3ccccc3)n2)cc1 ; c1ccc(-c2cc(-c3ccc(-n4c5cccc(-c6ccccc6)c5c5ccc6sc7ccccc7c6c54)cc3)nc(-c3ccccc3)n2)cc1 ; c1ccc(-c2ccc3c(c2)c2ccc4sc5ccccc5c4c2n3-c2ccc(-c3cc(-c4ccccc4)nc(-c4ccccc4)n3)cc2)cc1]

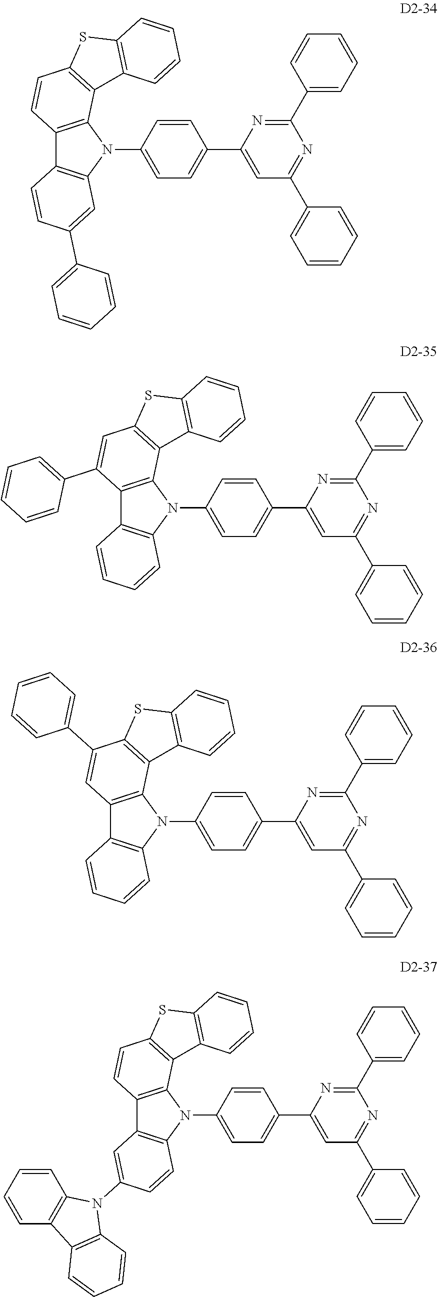 [SMILES: c1ccc(-c2ccc3c4ccc5sc6ccccc6c5c4n(-c4ccc(-c5cc(-c6ccccc6)nc(-c6ccccc6)n5)cc4)c3c2)cc1 ; c1ccc(-c2cc(-c3ccc(-n4c5ccccc5c5c(-c6ccccc6)cc6sc7ccccc7c6c54)cc3)nc(-c3ccccc3)n2)cc1 ; c1ccc(-c2cc(-c3ccc(-n4c5ccccc5c5cc(-c6ccccc6)c6sc7ccccc7c6c54)cc3)nc(-c3ccccc3)n2)cc1 ; c1ccc(-c2cc(-c3ccc(-n4c5ccc(-n6c7ccccc7c7ccccc76)cc5c5ccc6sc7ccccc7c6c54)cc3)nc(-c3ccccc3)n2)cc1]

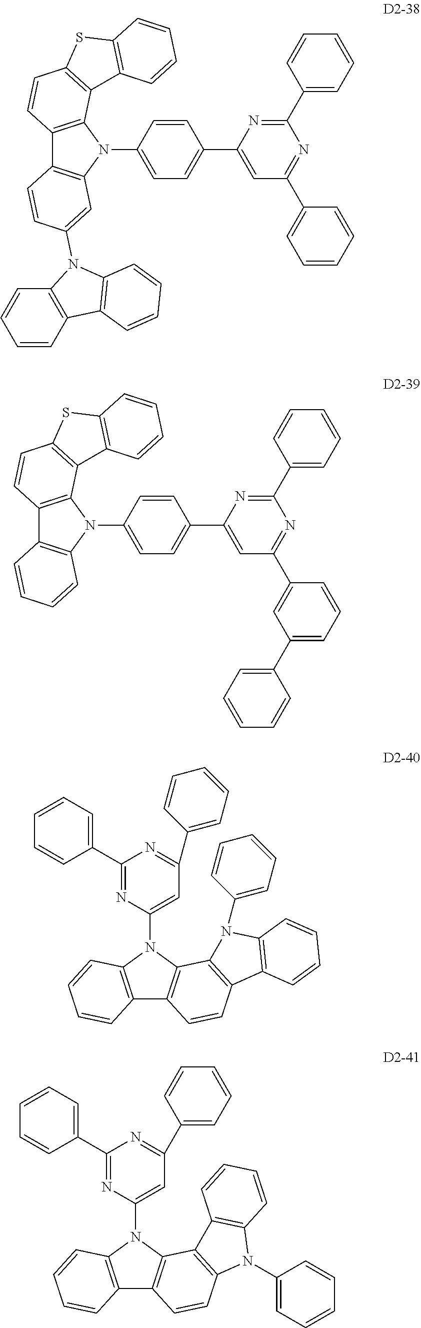

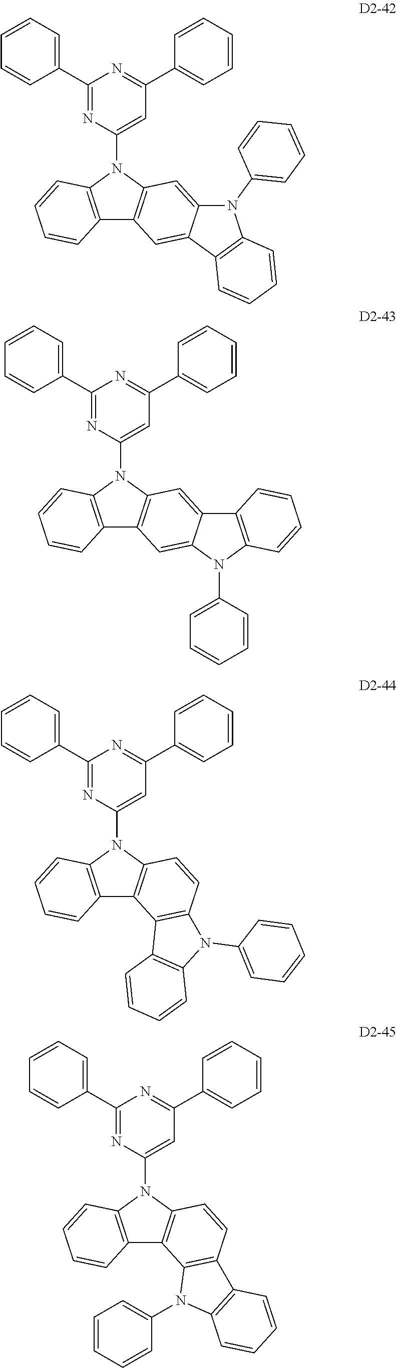

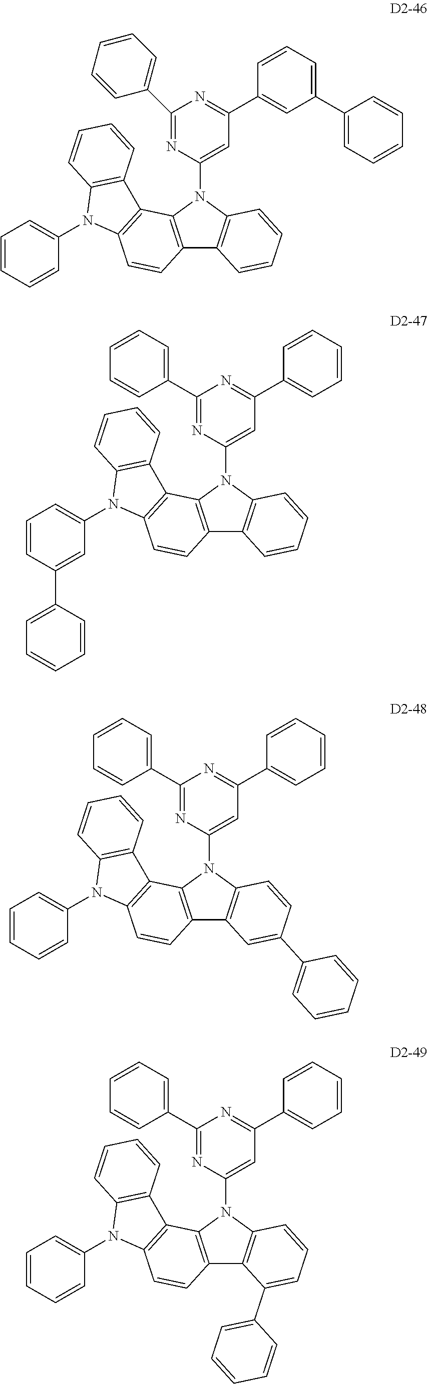

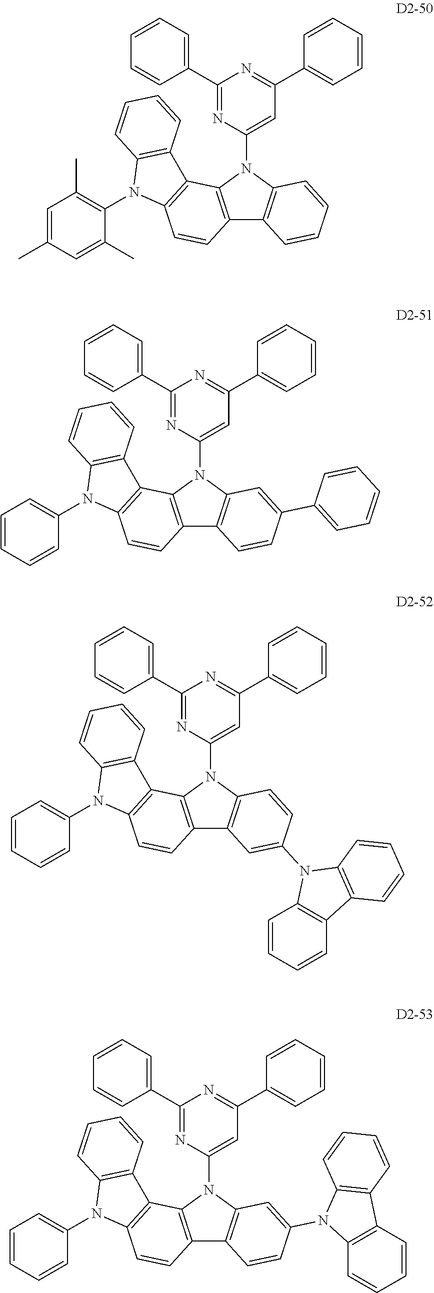 [SMILES: Cc1cc(C)c(-n2c3ccccc3c3c2ccc2c4ccccc4n(-c4cc(-c5ccccc5)nc(-c5ccccc5)n4)c23)c(C)c1 ; c1ccc(-c2ccc3c4ccc5c(c6ccccc6n5-c5ccccc5)c4n(-c4cc(-c5ccccc5)nc(-c5ccccc5)n4)c3c2)cc1 ; c1ccc(-c2cc(-n3c4ccc(-n5c6ccccc6c6ccccc65)cc4c4ccc5c(c6ccccc6n5-c5ccccc5)c43)nc(-c3ccccc3)n2)cc1 ; c1ccc(-c2cc(-n3c4cc(-n5c6ccccc6c6ccccc65)ccc4c4ccc5c(c6ccccc6n5-c5ccccc5)c43)nc(-c3ccccc3)n2)cc1]

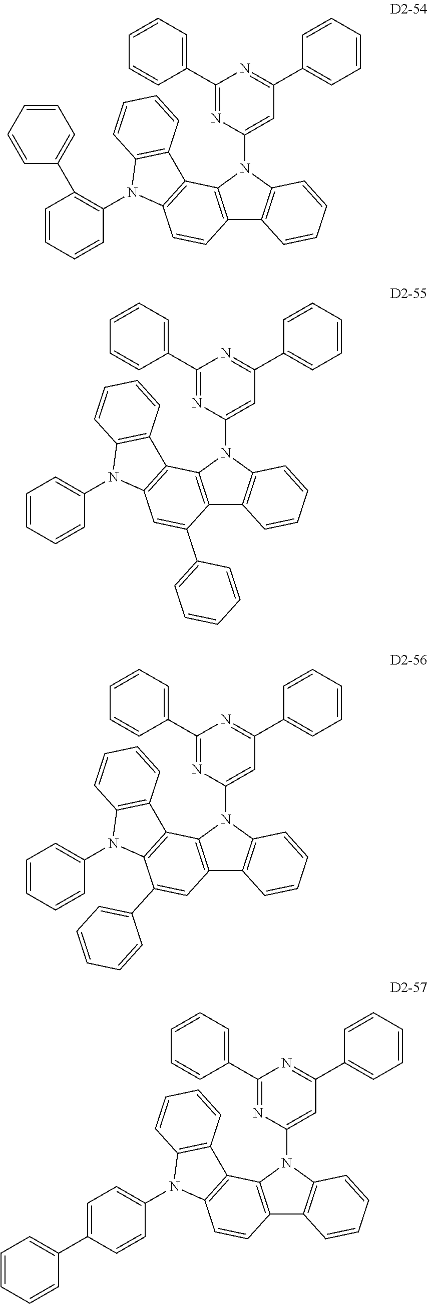 [SMILES: c1ccc(-c2cc(-n3c4ccccc4c4ccc5c(c6ccccc6n5-c5ccccc5-c5ccccc5)c43)nc(-c3ccccc3)n2)cc1 ; c1ccc(-c2cc(-n3c4ccccc4c4c(-c5ccccc5)cc5c(c6ccccc6n5-c5ccccc5)c43)nc(-c3ccccc3)n2)cc1 ; c1ccc(-c2cc(-n3c4ccccc4c4cc(-c5ccccc5)c5c(c6ccccc6n5-c5ccccc5)c43)nc(-c3ccccc3)n2)cc1 ; c1ccc(-c2ccc(-n3c4ccccc4c4c3ccc3c5ccccc5n(-c5cc(-c6ccccc6)nc(-c6ccccc6)n5)c34)cc2)cc1]

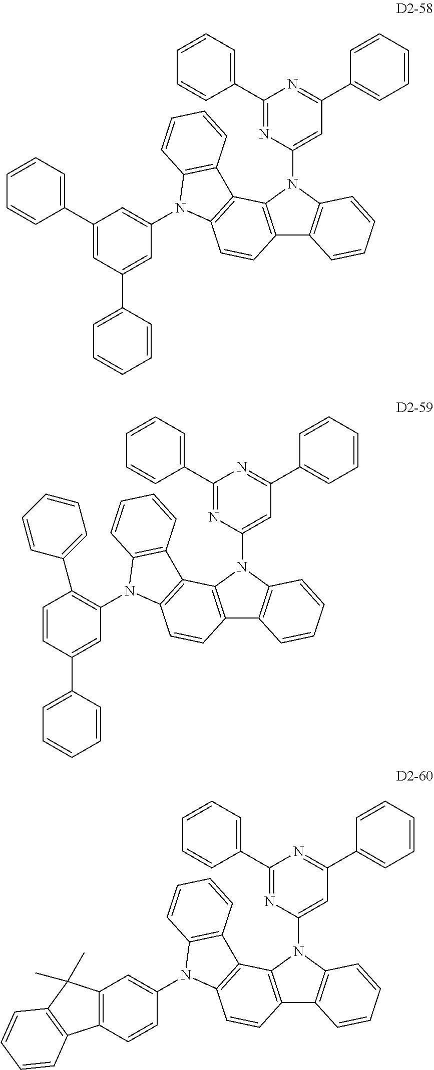 [SMILES: c1ccc(-c2cc(-c3ccccc3)cc(-n3c4ccccc4c4c3ccc3c5ccccc5n(-c5cc(-c6ccccc6)nc(-c6ccccc6)n5)c34)c2)cc1 ; c1ccc(-c2ccc(-c3ccccc3)c(-n3c4ccccc4c4c3ccc3c5ccccc5n(-c5cc(-c6ccccc6)nc(-c6ccccc6)n5)c34)c2)cc1 ; CC1(C)c2ccccc2-c2ccc(-n3c4ccccc4c4c3ccc3c5ccccc5n(-c5cc(-c6ccccc6)nc(-c6ccccc6)n5)c34)cc21]

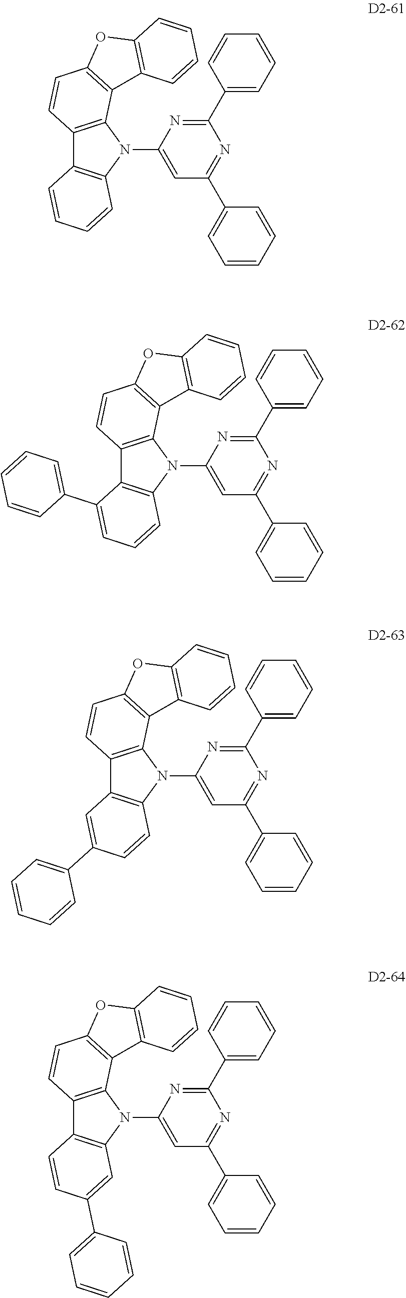 [SMILES: c1ccc(-c2cc(-n3c4ccccc4c4ccc5oc6ccccc6c5c43)nc(-c3ccccc3)n2)cc1 ; c1ccc(-c2cc(-n3c4cccc(-c5ccccc5)c4c4ccc5oc6ccccc6c5c43)nc(-c3ccccc3)n2)cc1 ; c1ccc(-c2ccc3c(c2)c2ccc4oc5ccccc5c4c2n3-c2cc(-c3ccccc3)nc(-c3ccccc3)n2)cc1 ; c1ccc(-c2ccc3c4ccc5oc6ccccc6c5c4n(-c4cc(-c5ccccc5)nc(-c5ccccc5)n4)c3c2)cc1]

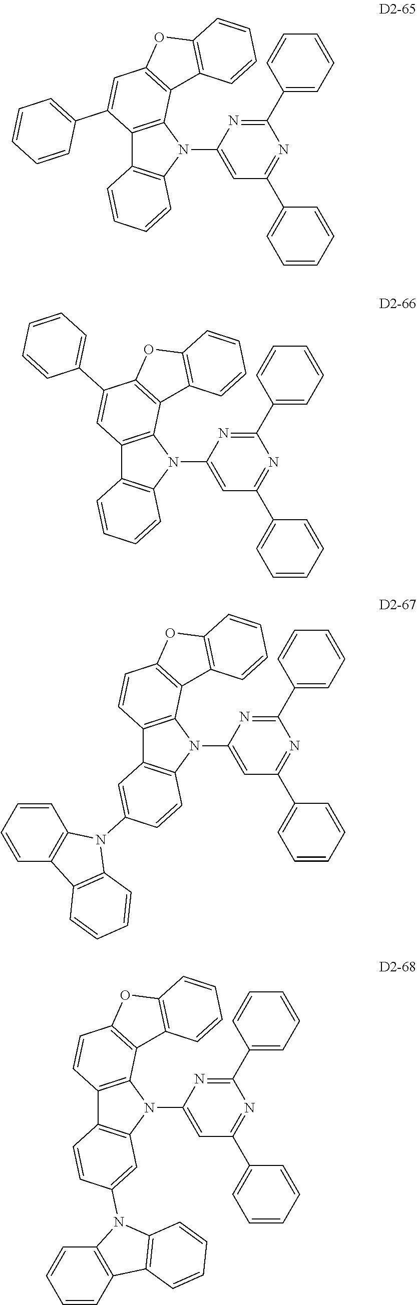 [SMILES: c1ccc(-c2cc(-n3c4ccccc4c4c(-c5ccccc5)cc5oc6ccccc6c5c43)nc(-c3ccccc3)n2)cc1 ; c1ccc(-c2cc(-n3c4ccccc4c4cc(-c5ccccc5)c5oc6ccccc6c5c43)nc(-c3ccccc3)n2)cc1 ; c1ccc(-c2cc(-n3c4ccc(-n5c6ccccc6c6ccccc65)cc4c4ccc5oc6ccccc6c5c43)nc(-c3ccccc3)n2)cc1 ; c1ccc(-c2cc(-n3c4cc(-n5c6ccccc6c6ccccc65)ccc4c4ccc5oc6ccccc6c5c43)nc(-c3ccccc3)n2)cc1]

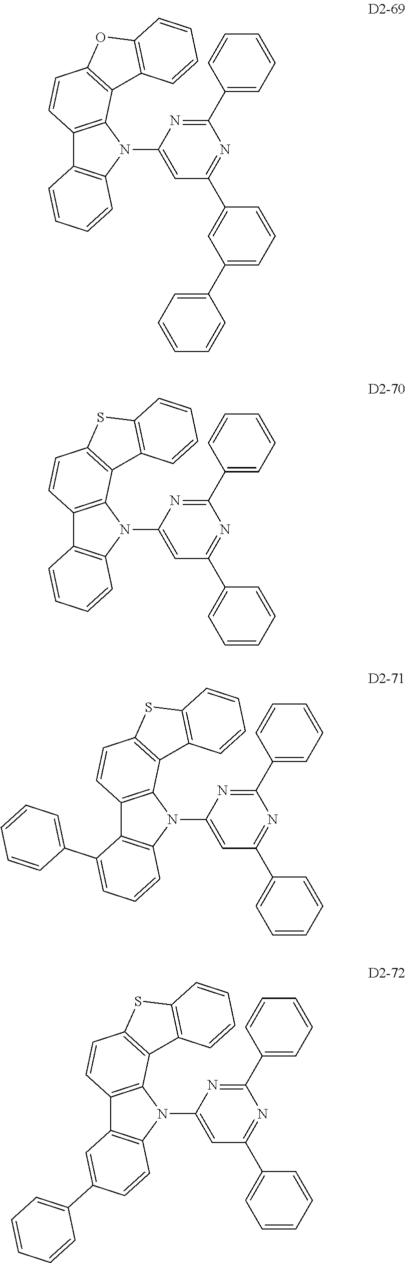 [SMILES: c1ccc(-c2cccc(-c3cc(-n4c5ccccc5c5ccc6oc7ccccc7c6c54)nc(-c4ccccc4)n3)c2)cc1 ; c1ccc(-c2cc(-n3c4ccccc4c4ccc5sc6ccccc6c5c43)nc(-c3ccccc3)n2)cc1 ; c1ccc(-c2cc(-n3c4cccc(-c5ccccc5)c4c4ccc5sc6ccccc6c5c43)nc(-c3ccccc3)n2)cc1 ; c1ccc(-c2ccc3c(c2)c2ccc4sc5ccccc5c4c2n3-c2cc(-c3ccccc3)nc(-c3ccccc3)n2)cc1]

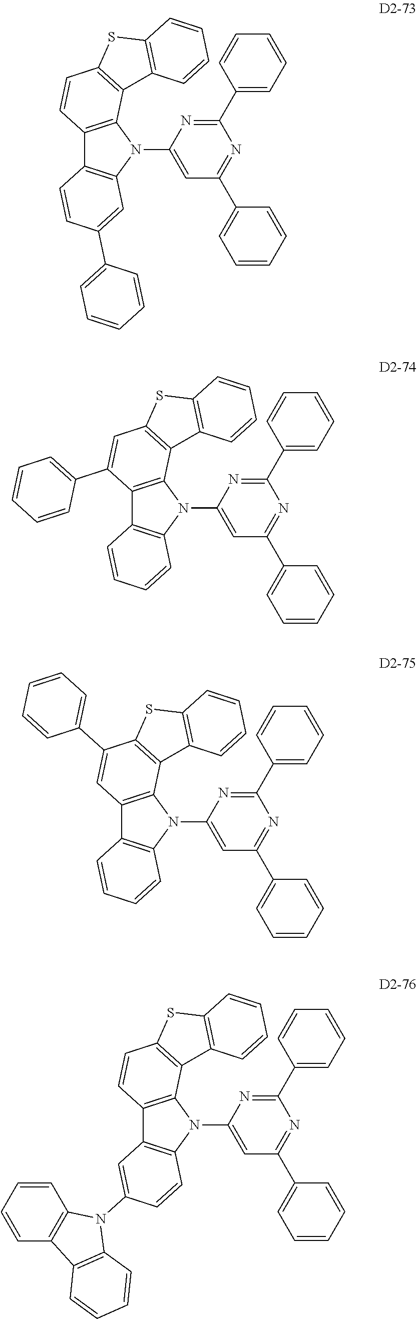 [SMILES: c1ccc(-c2ccc3c4ccc5sc6ccccc6c5c4n(-c4cc(-c5ccccc5)nc(-c5ccccc5)n4)c3c2)cc1 ; c1ccc(-c2cc(-n3c4ccccc4c4c(-c5ccccc5)cc5sc6ccccc6c5c43)nc(-c3ccccc3)n2)cc1 ; c1ccc(-c2cc(-n3c4ccccc4c4cc(-c5ccccc5)c5sc6ccccc6c5c43)nc(-c3ccccc3)n2)cc1 ; c1ccc(-c2cc(-n3c4ccc(-n5c6ccccc6c6ccccc65)cc4c4ccc5sc6ccccc6c5c43)nc(-c3ccccc3)n2)cc1]

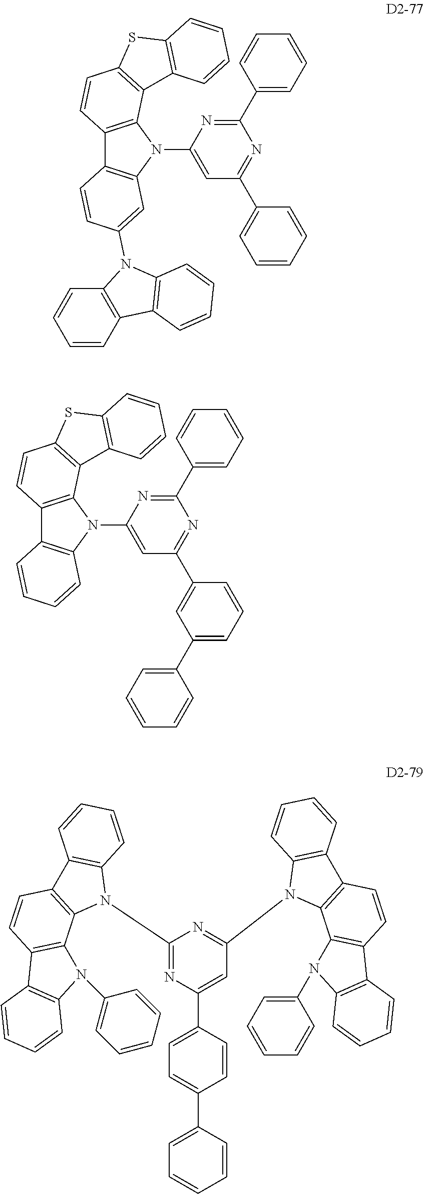 [SMILES: c1ccc(-c2cc(-n3c4cc(-n5c6ccccc6c6ccccc65)ccc4c4ccc5sc6ccccc6c5c43)nc(-c3ccccc3)n2)cc1 ; c1ccc(-c2cccc(-c3cc(-n4c5ccccc5c5ccc6sc7ccccc7c6c54)nc(-c4ccccc4)n3)c2)cc1 ; c1ccc(-c2ccc(-c3cc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1]

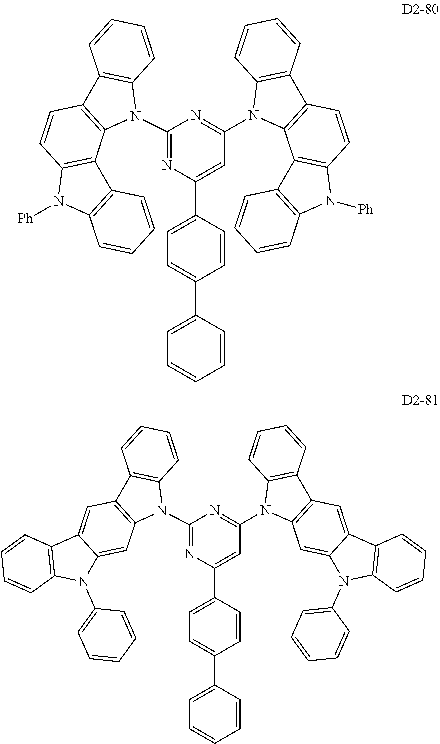 [SMILES: c1ccc(-c2ccc(-c3cc(-n4c5ccccc5c5ccc6c(c7ccccc7n6-c6ccccc6)c54)nc(-n4c5ccccc5c5ccc6c(c7ccccc7n6-c6ccccc6)c54)n3)cc2)cc1 ; c1ccc(-c2ccc(-c3cc(-n4c5ccccc5c5cc6c7ccccc7n(-c7ccccc7)c6cc54)nc(-n4c5ccccc5c5cc6c7ccccc7n(-c7ccccc7)c6cc54)n3)cc2)cc1]

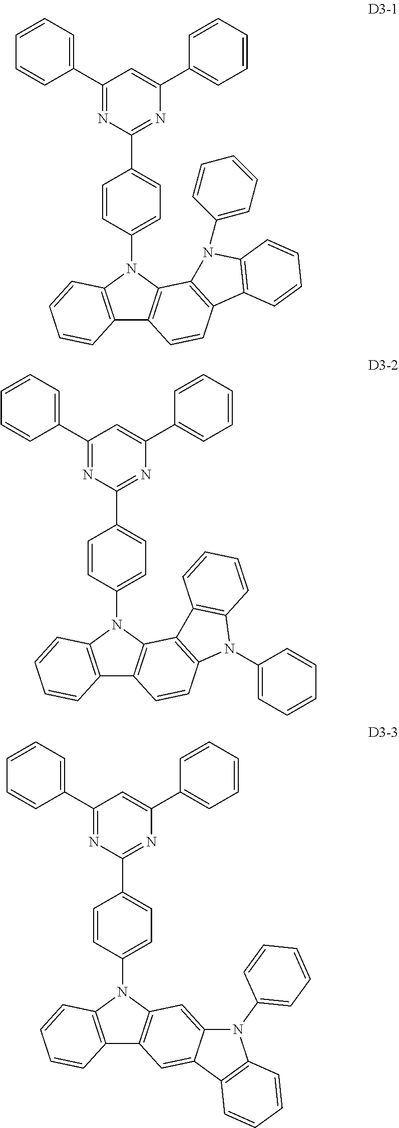

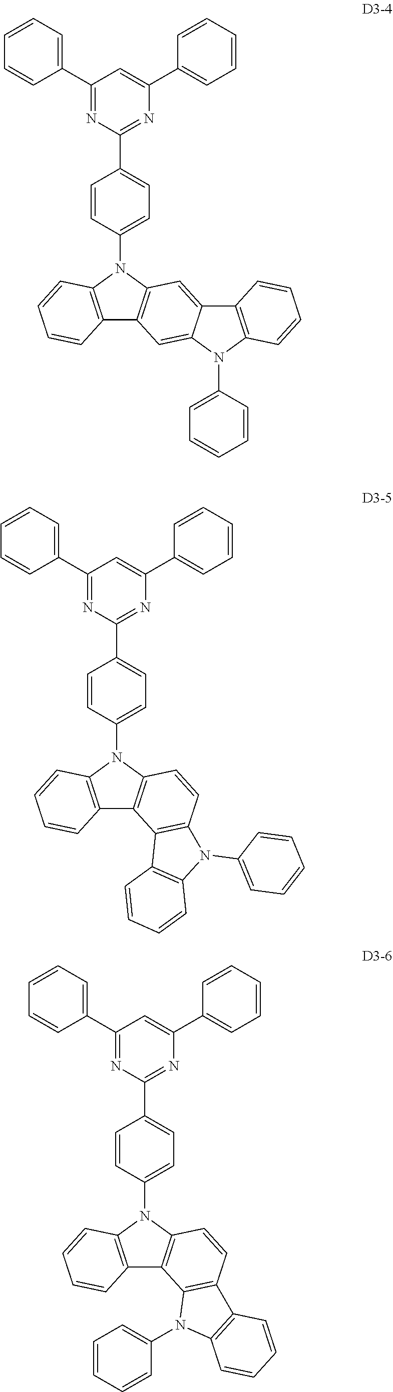 [SMILES: c1ccc(-c2cc(-c3ccccc3)nc(-c3ccc(-n4c5ccccc5c5cc6c(cc54)c4ccccc4n6-c4ccccc4)cc3)n2)cc1 ; c1ccc(-c2cc(-c3ccccc3)nc(-c3ccc(-n4c5ccccc5c5c6c7ccccc7n(-c7ccccc7)c6ccc54)cc3)n2)cc1 ; c1ccc(-c2cc(-c3ccccc3)nc(-c3ccc(-n4c5ccccc5c5c4ccc4c6ccccc6n(-c6ccccc6)c45)cc3)n2)cc1]

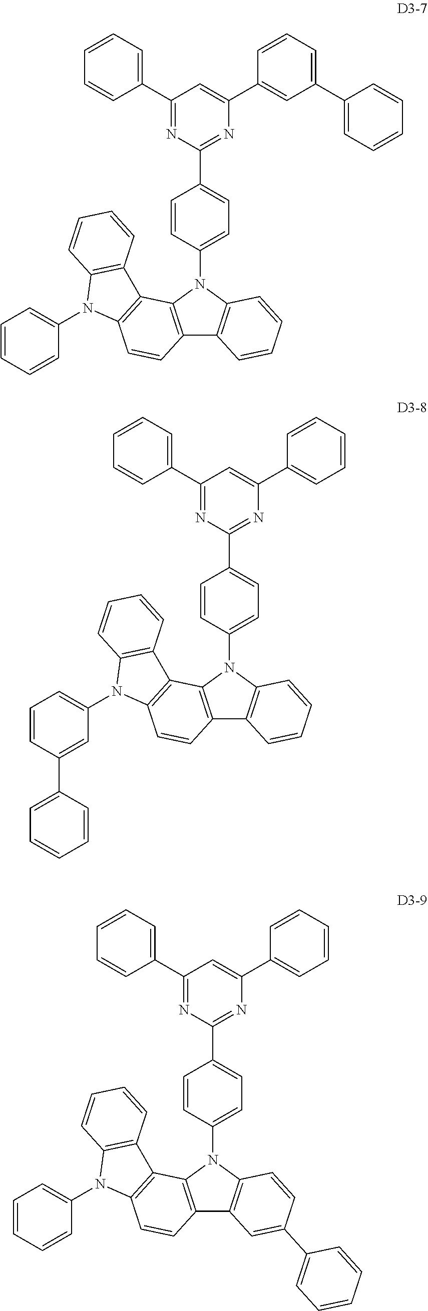

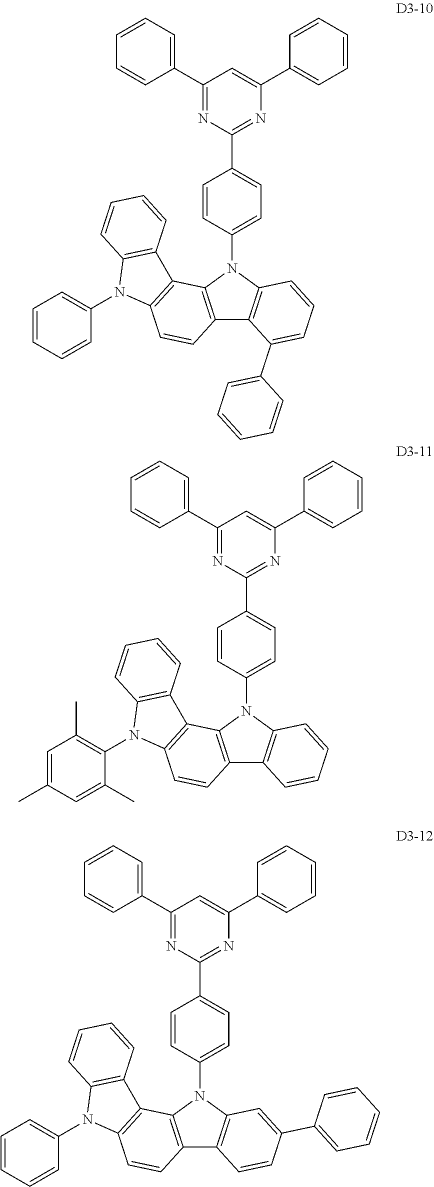 [SMILES: c1ccc(-c2cc(-c3ccccc3)nc(-c3ccc(-n4c5cccc(-c6ccccc6)c5c5ccc6c(c7ccccc7n6-c6ccccc6)c54)cc3)n2)cc1 ; Cc1cc(C)c(-n2c3ccccc3c3c2ccc2c4ccccc4n(-c4ccc(-c5nc(-c6ccccc6)cc(-c6ccccc6)n5)cc4)c23)c(C)c1 ; c1ccc(-c2ccc3c4ccc5c(c6ccccc6n5-c5ccccc5)c4n(-c4ccc(-c5nc(-c6ccccc6)cc(-c6ccccc6)n5)cc4)c3c2)cc1]

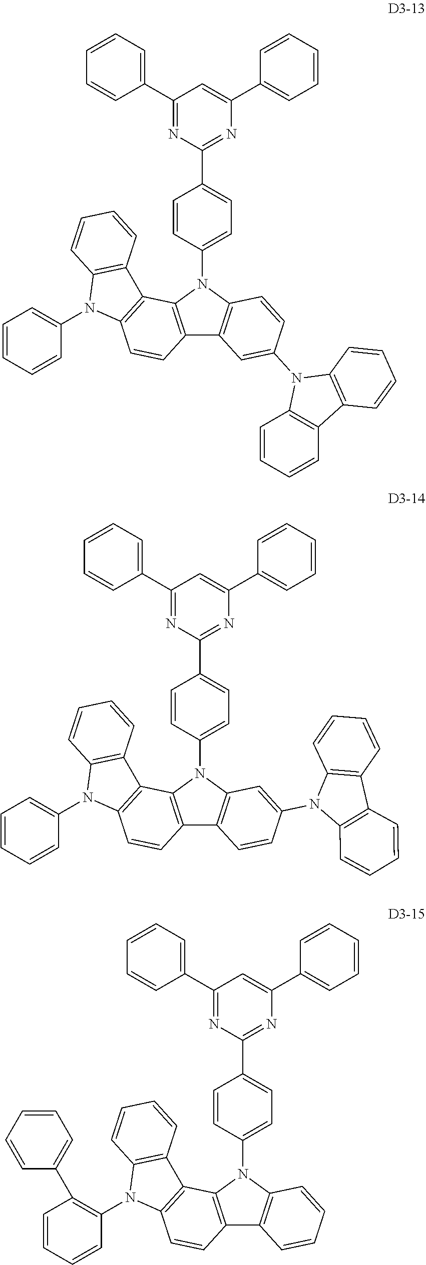

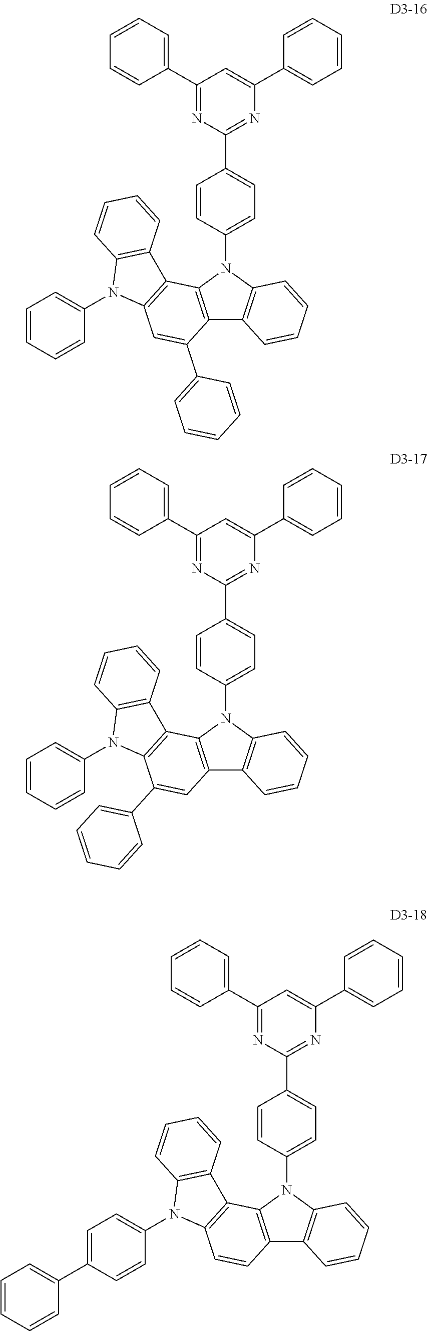 [SMILES: c1ccc(-c2cc(-c3ccccc3)nc(-c3ccc(-n4c5ccccc5c5c(-c6ccccc6)cc6c(c7ccccc7n6-c6ccccc6)c54)cc3)n2)cc1 ; c1ccc(-c2cc(-c3ccccc3)nc(-c3ccc(-n4c5ccccc5c5cc(-c6ccccc6)c6c(c7ccccc7n6-c6ccccc6)c54)cc3)n2)cc1 ; c1ccc(-c2ccc(-n3c4ccccc4c4c3ccc3c5ccccc5n(-c5ccc(-c6nc(-c7ccccc7)cc(-c7ccccc7)n6)cc5)c34)cc2)cc1]

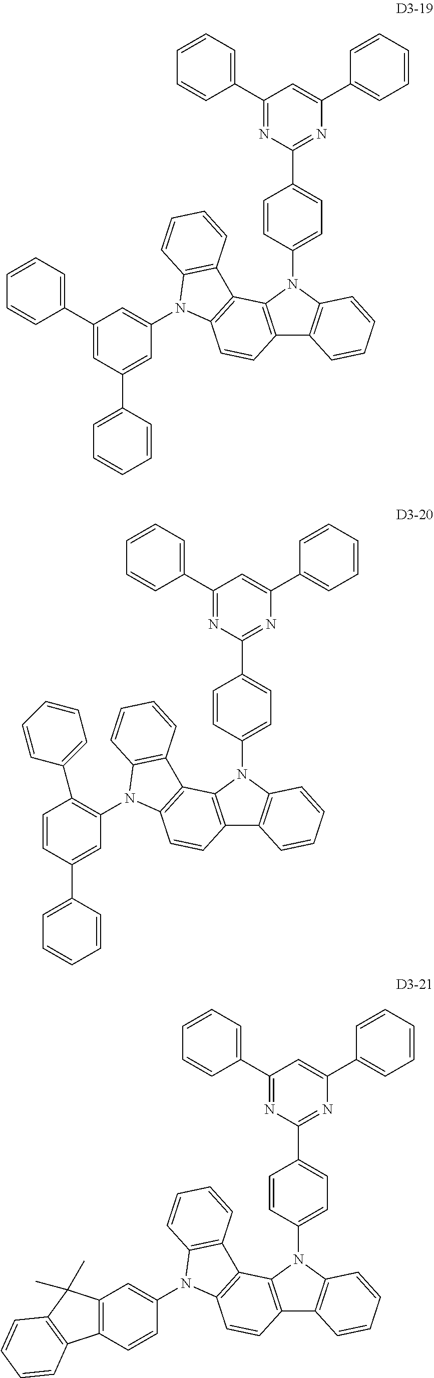 [SMILES: c1ccc(-c2cc(-c3ccccc3)cc(-n3c4ccccc4c4c3ccc3c5ccccc5n(-c5ccc(-c6nc(-c7ccccc7)cc(-c7ccccc7)n6)cc5)c34)c2)cc1 ; c1ccc(-c2ccc(-c3ccccc3)c(-n3c4ccccc4c4c3ccc3c5ccccc5n(-c5ccc(-c6nc(-c7ccccc7)cc(-c7ccccc7)n6)cc5)c34)c2)cc1 ; CC1(C)c2ccccc2-c2ccc(-n3c4ccccc4c4c3ccc3c5ccccc5n(-c5ccc(-c6nc(-c7ccccc7)cc(-c7ccccc7)n6)cc5)c34)cc21]

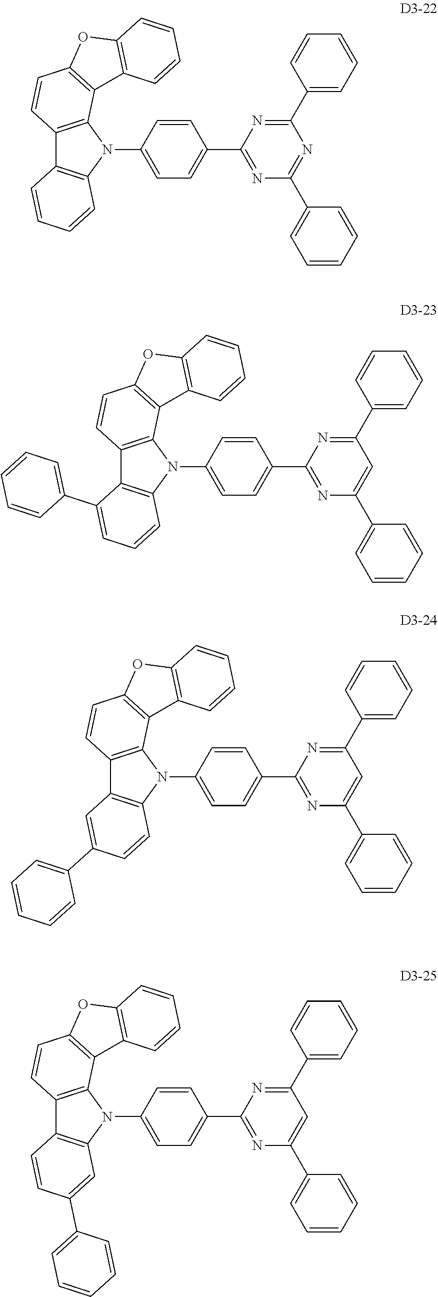 [SMILES: c1ccc(-c2nc(-c3ccccc3)nc(-c3ccc(-n4c5ccccc5c5ccc6oc7ccccc7c6c54)cc3)n2)cc1 ; c1ccc(-c2cc(-c3ccccc3)nc(-c3ccc(-n4c5cccc(-c6ccccc6)c5c5ccc6oc7ccccc7c6c54)cc3)n2)cc1 ; c1ccc(-c2ccc3c(c2)c2ccc4oc5ccccc5c4c2n3-c2ccc(-c3nc(-c4ccccc4)cc(-c4ccccc4)n3)cc2)cc1 ; c1ccc(-c2ccc3c4ccc5oc6ccccc6c5c4n(-c4ccc(-c5nc(-c6ccccc6)cc(-c6ccccc6)n5)cc4)c3c2)cc1]

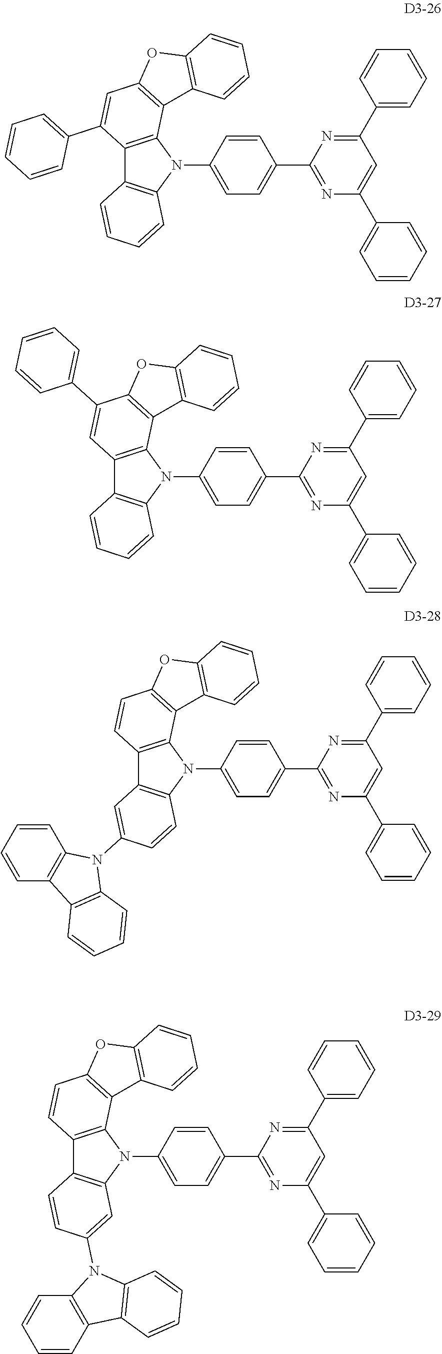 [SMILES: c1ccc(-c2cc(-c3ccccc3)nc(-c3ccc(-n4c5ccccc5c5c(-c6ccccc6)cc6oc7ccccc7c6c54)cc3)n2)cc1 ; c1ccc(-c2cc(-c3ccccc3)nc(-c3ccc(-n4c5ccccc5c5cc(-c6ccccc6)c6oc7ccccc7c6c54)cc3)n2)cc1 ; c1ccc(-c2cc(-c3ccccc3)nc(-c3ccc(-n4c5ccc(-n6c7ccccc7c7ccccc76)cc5c5ccc6oc7ccccc7c6c54)cc3)n2)cc1 ; c1ccc(-c2cc(-c3ccccc3)nc(-c3ccc(-n4c5cc(-n6c7ccccc7c7ccccc76)ccc5c5ccc6oc7ccccc7c6c54)cc3)n2)cc1]

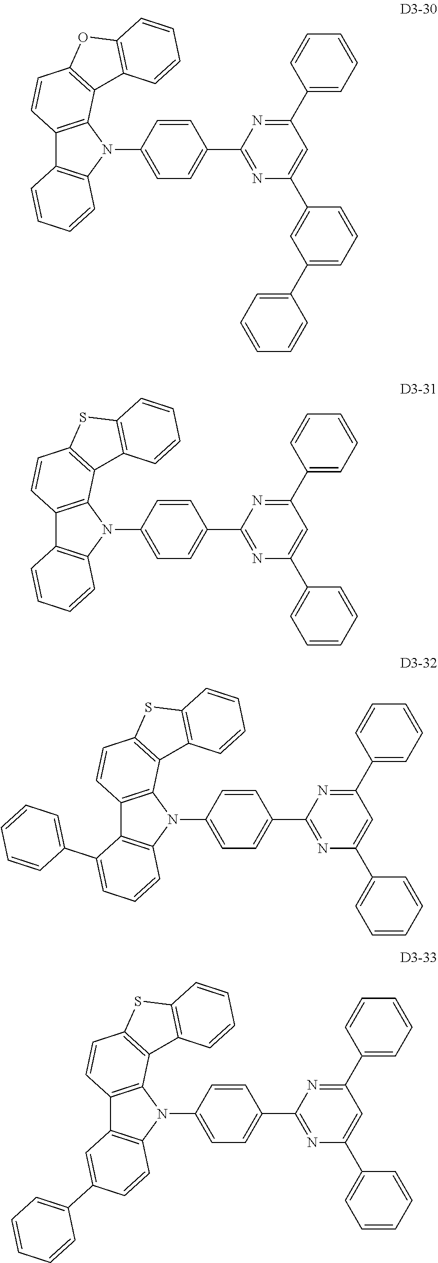 [SMILES: c1ccc(-c2cccc(-c3cc(-c4ccccc4)nc(-c4ccc(-n5c6ccccc6c6ccc7oc8ccccc8c7c65)cc4)n3)c2)cc1 ; c1ccc(-c2cc(-c3ccccc3)nc(-c3ccc(-n4c5ccccc5c5ccc6sc7ccccc7c6c54)cc3)n2)cc1 ; c1ccc(-c2cc(-c3ccccc3)nc(-c3ccc(-n4c5cccc(-c6ccccc6)c5c5ccc6sc7ccccc7c6c54)cc3)n2)cc1 ; c1ccc(-c2ccc3c(c2)c2ccc4sc5ccccc5c4c2n3-c2ccc(-c3nc(-c4ccccc4)cc(-c4ccccc4)n3)cc2)cc1]

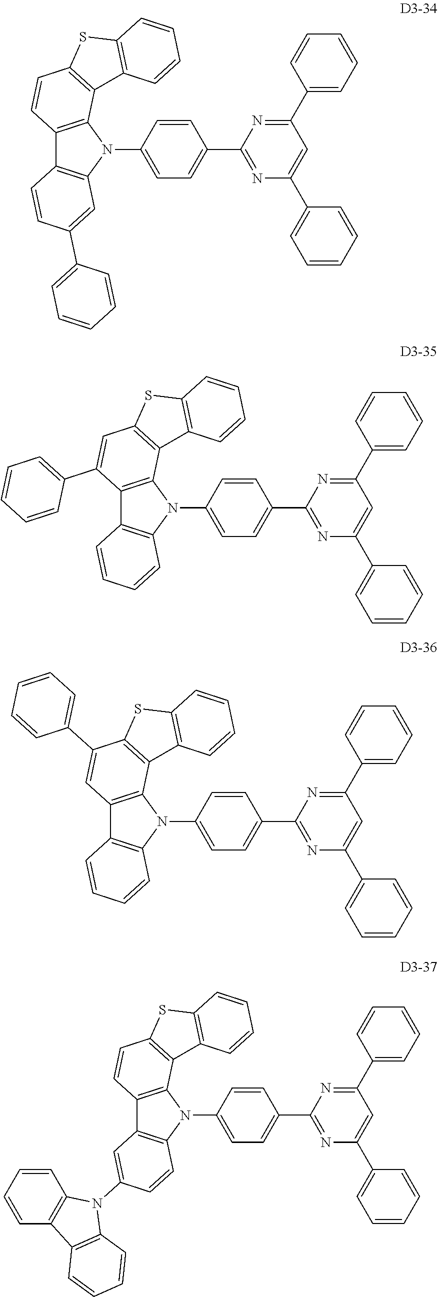 [SMILES: c1ccc(-c2ccc3c4ccc5sc6ccccc6c5c4n(-c4ccc(-c5nc(-c6ccccc6)cc(-c6ccccc6)n5)cc4)c3c2)cc1 ; c1ccc(-c2cc(-c3ccccc3)nc(-c3ccc(-n4c5ccccc5c5c(-c6ccccc6)cc6sc7ccccc7c6c54)cc3)n2)cc1 ; c1ccc(-c2cc(-c3ccccc3)nc(-c3ccc(-n4c5ccccc5c5cc(-c6ccccc6)c6sc7ccccc7c6c54)cc3)n2)cc1 ; c1ccc(-c2cc(-c3ccccc3)nc(-c3ccc(-n4c5ccc(-n6c7ccccc7c7ccccc76)cc5c5ccc6sc7ccccc7c6c54)cc3)n2)cc1]

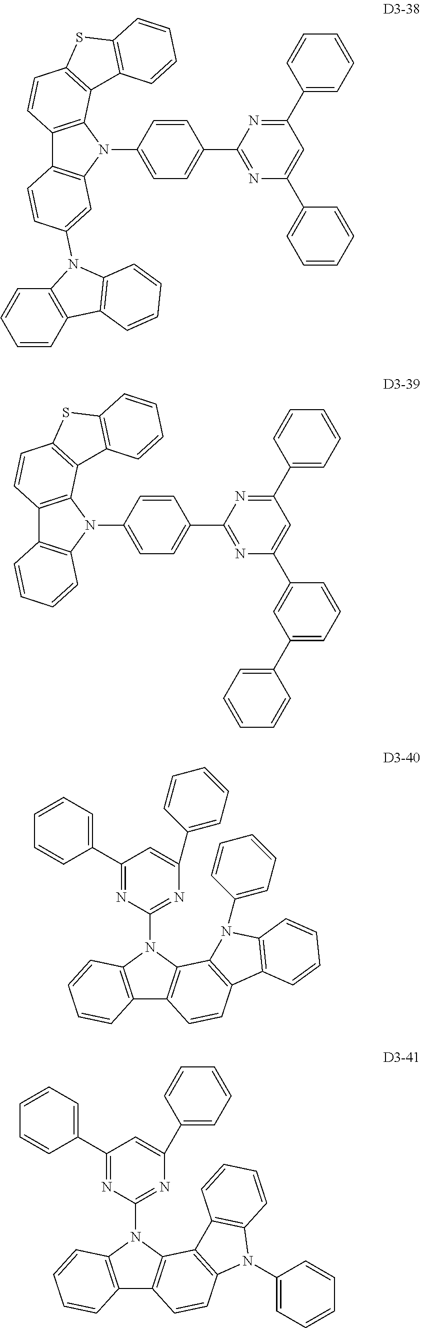

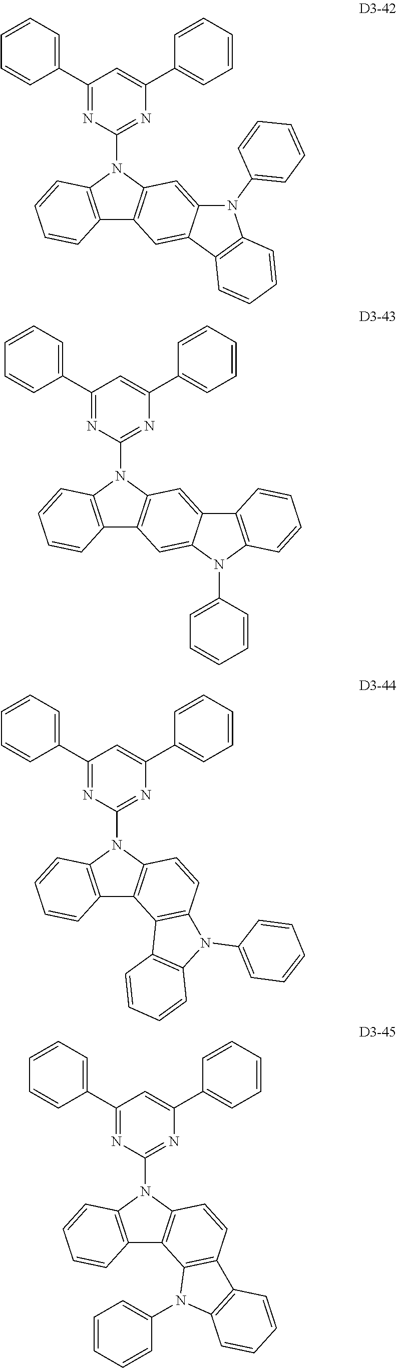

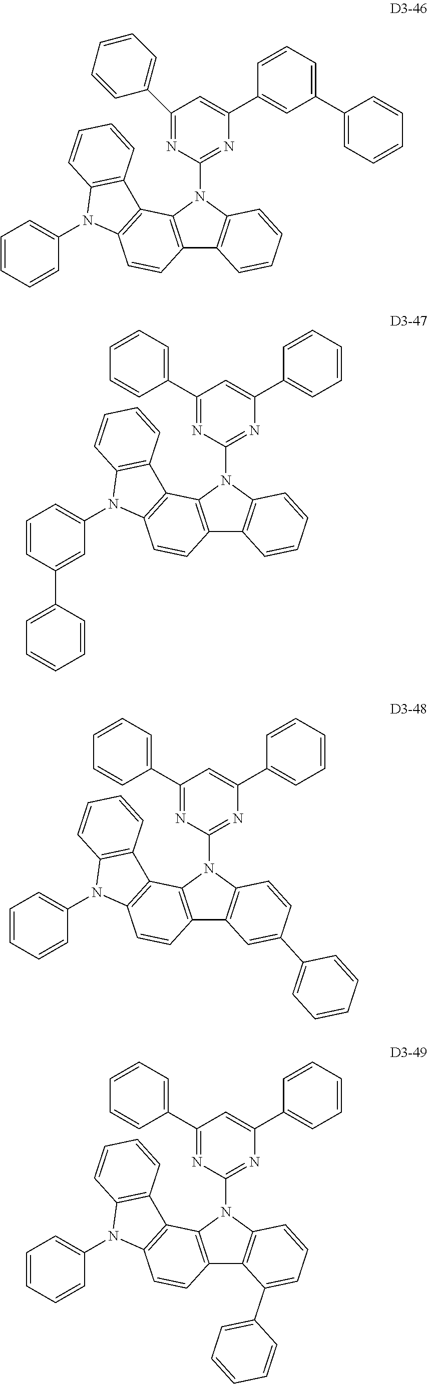

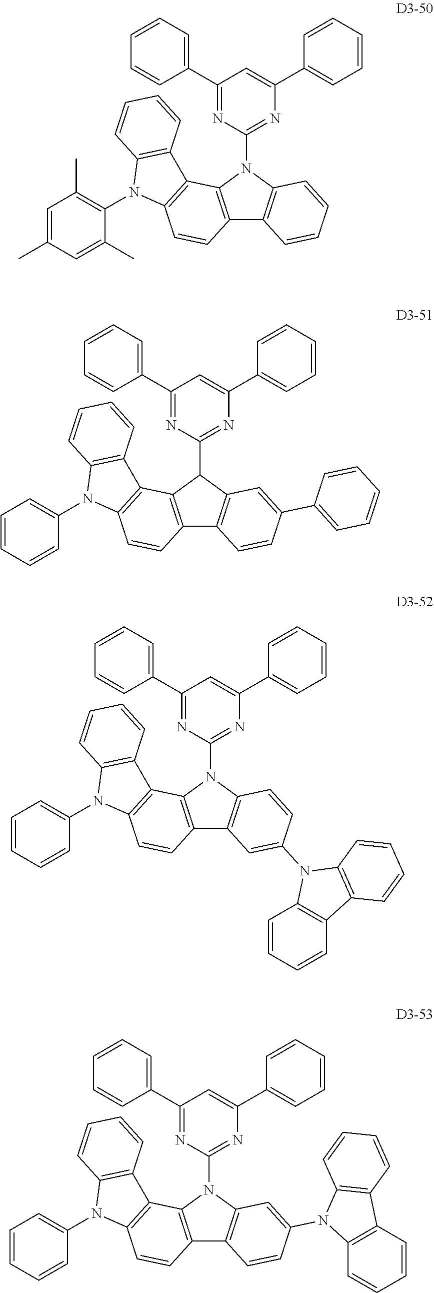 [SMILES: Cc1cc(C)c(-n2c3ccccc3c3c2ccc2c4ccccc4n(-c4nc(-c5ccccc5)cc(-c5ccccc5)n4)c23)c(C)c1 ; c1ccc(-c2ccc3c(c2)C(c2nc(-c4ccccc4)cc(-c4ccccc4)n2)c2c-3ccc3c2c2ccccc2n3-c2ccccc2)cc1 ; c1ccc(-c2cc(-c3ccccc3)nc(-n3c4ccc(-n5c6ccccc6c6ccccc65)cc4c4ccc5c(c6ccccc6n5-c5ccccc5)c43)n2)cc1 ; c1ccc(-c2cc(-c3ccccc3)nc(-n3c4cc(-n5c6ccccc6c6ccccc65)ccc4c4ccc5c(c6ccccc6n5-c5ccccc5)c43)n2)cc1]

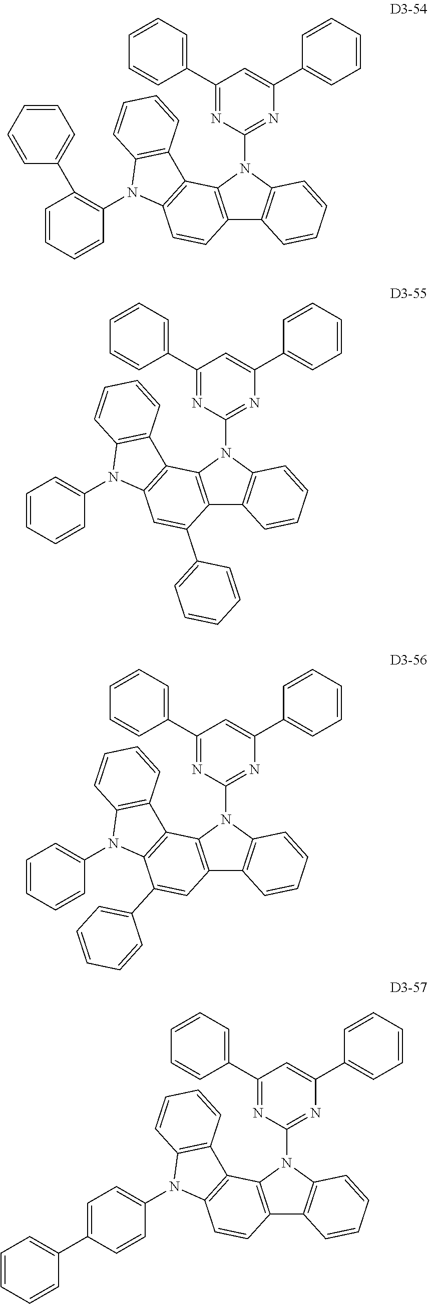 [SMILES: c1ccc(-c2cc(-c3ccccc3)nc(-n3c4ccccc4c4ccc5c(c6ccccc6n5-c5ccccc5-c5ccccc5)c43)n2)cc1 ; c1ccc(-c2cc(-c3ccccc3)nc(-n3c4ccccc4c4c(-c5ccccc5)cc5c(c6ccccc6n5-c5ccccc5)c43)n2)cc1 ; c1ccc(-c2cc(-c3ccccc3)nc(-n3c4ccccc4c4cc(-c5ccccc5)c5c(c6ccccc6n5-c5ccccc5)c43)n2)cc1 ; c1ccc(-c2ccc(-n3c4ccccc4c4c3ccc3c5ccccc5n(-c5nc(-c6ccccc6)cc(-c6ccccc6)n5)c34)cc2)cc1]

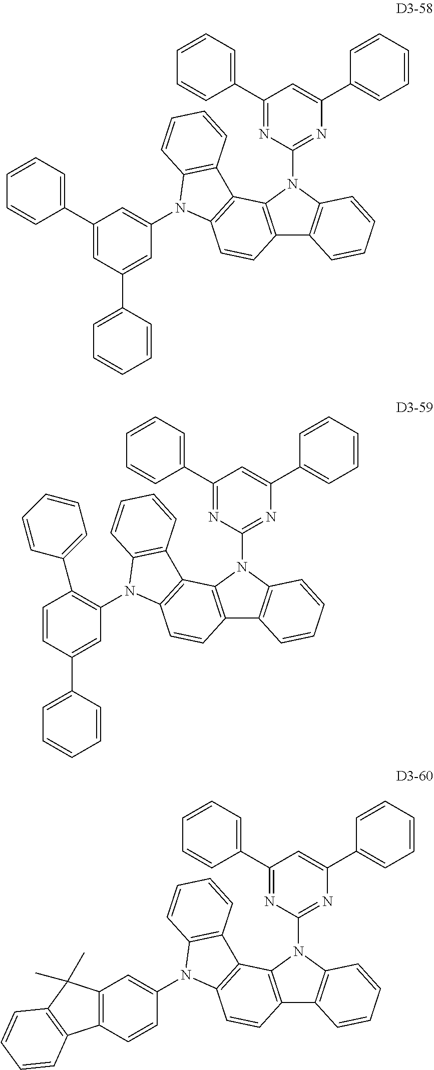 [SMILES: c1ccc(-c2cc(-c3ccccc3)cc(-n3c4ccccc4c4c3ccc3c5ccccc5n(-c5nc(-c6ccccc6)cc(-c6ccccc6)n5)c34)c2)cc1 ; c1ccc(-c2ccc(-c3ccccc3)c(-n3c4ccccc4c4c3ccc3c5ccccc5n(-c5nc(-c6ccccc6)cc(-c6ccccc6)n5)c34)c2)cc1 ; CC1(C)c2ccccc2-c2ccc(-n3c4ccccc4c4c3ccc3c5ccccc5n(-c5nc(-c6ccccc6)cc(-c6ccccc6)n5)c34)cc21]

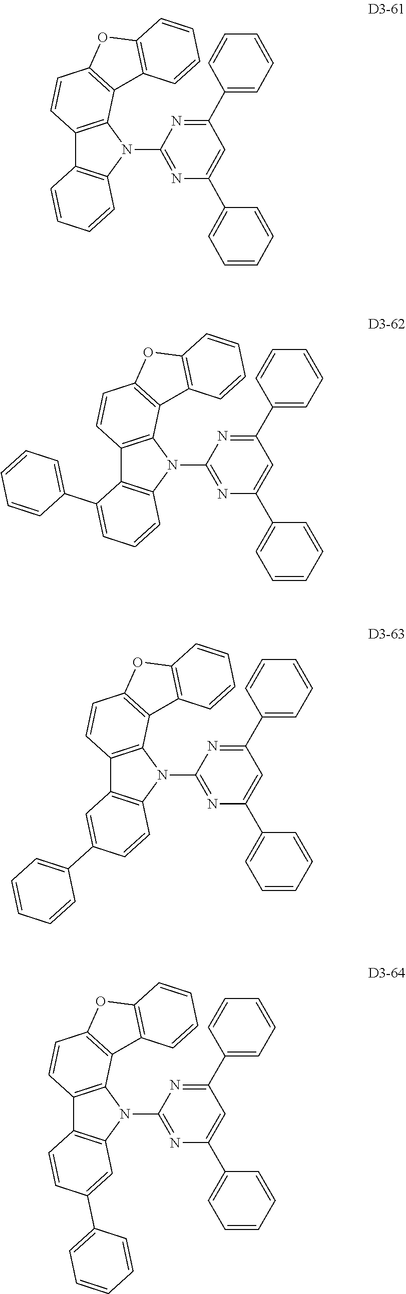 [SMILES: c1ccc(-c2cc(-c3ccccc3)nc(-n3c4ccccc4c4ccc5oc6ccccc6c5c43)n2)cc1 ; c1ccc(-c2cc(-c3ccccc3)nc(-n3c4cccc(-c5ccccc5)c4c4ccc5oc6ccccc6c5c43)n2)cc1 ; c1ccc(-c2ccc3c(c2)c2ccc4oc5ccccc5c4c2n3-c2nc(-c3ccccc3)cc(-c3ccccc3)n2)cc1 ; c1ccc(-c2ccc3c4ccc5oc6ccccc6c5c4n(-c4nc(-c5ccccc5)cc(-c5ccccc5)n4)c3c2)cc1]

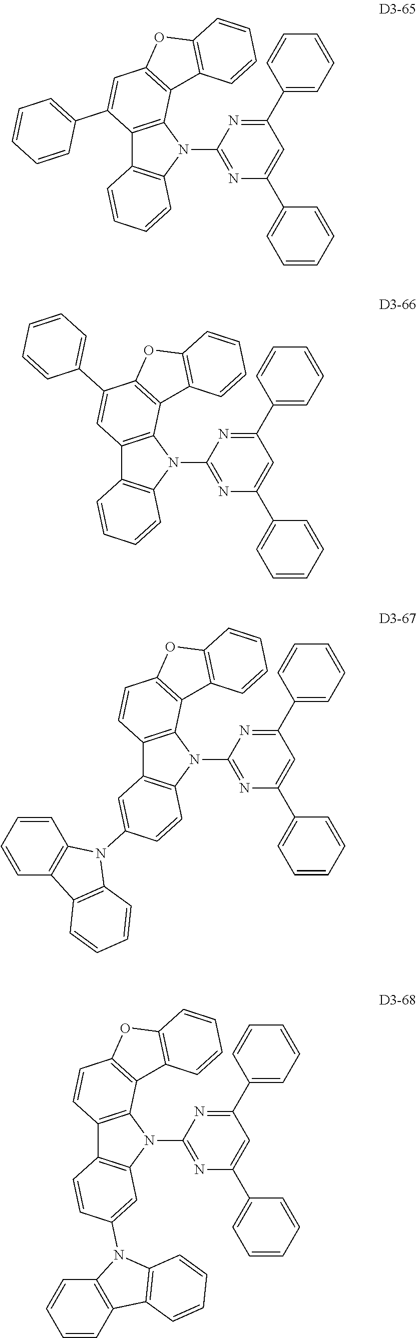 [SMILES: c1ccc(-c2cc(-c3ccccc3)nc(-n3c4ccccc4c4c(-c5ccccc5)cc5oc6ccccc6c5c43)n2)cc1 ; c1ccc(-c2cc(-c3ccccc3)nc(-n3c4ccccc4c4cc(-c5ccccc5)c5oc6ccccc6c5c43)n2)cc1 ; c1ccc(-c2cc(-c3ccccc3)nc(-n3c4ccc(-n5c6ccccc6c6ccccc65)cc4c4ccc5oc6ccccc6c5c43)n2)cc1 ; c1ccc(-c2cc(-c3ccccc3)nc(-n3c4cc(-n5c6ccccc6c6ccccc65)ccc4c4ccc5oc6ccccc6c5c43)n2)cc1]

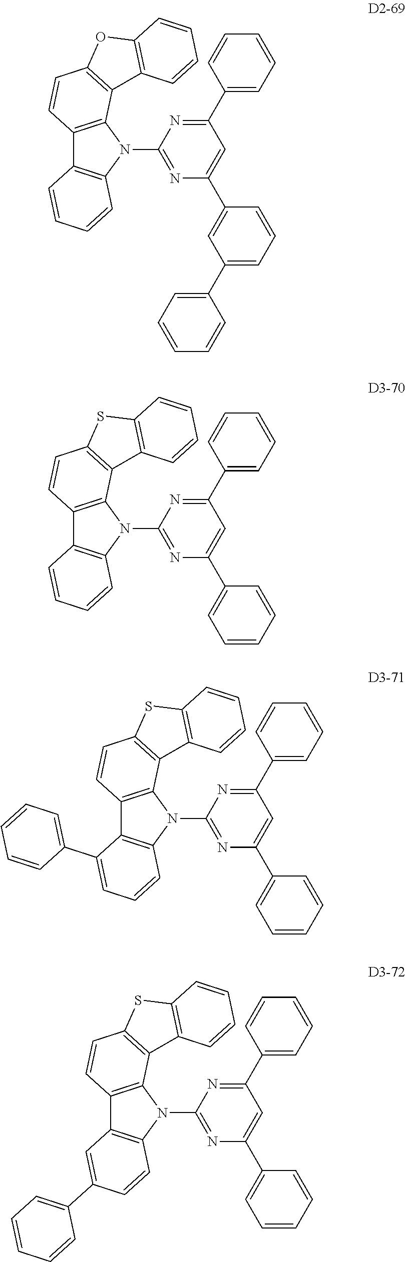 [SMILES: c1ccc(-c2cccc(-c3cc(-c4ccccc4)nc(-n4c5ccccc5c5ccc6oc7ccccc7c6c54)n3)c2)cc1 ; c1ccc(-c2cc(-c3ccccc3)nc(-n3c4ccccc4c4ccc5sc6ccccc6c5c43)n2)cc1 ; c1ccc(-c2cc(-c3ccccc3)nc(-n3c4cccc(-c5ccccc5)c4c4ccc5sc6ccccc6c5c43)n2)cc1 ; c1ccc(-c2ccc3c(c2)c2ccc4sc5ccccc5c4c2n3-c2nc(-c3ccccc3)cc(-c3ccccc3)n2)cc1]

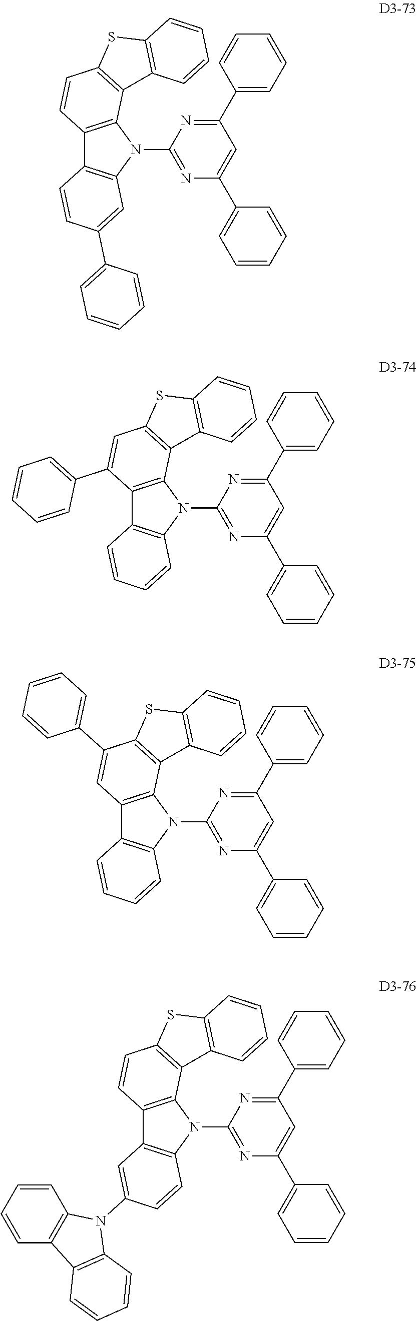 [SMILES: c1ccc(-c2ccc3c4ccc5sc6ccccc6c5c4n(-c4nc(-c5ccccc5)cc(-c5ccccc5)n4)c3c2)cc1 ; c1ccc(-c2cc(-c3ccccc3)nc(-n3c4ccccc4c4c(-c5ccccc5)cc5sc6ccccc6c5c43)n2)cc1 ; c1ccc(-c2cc(-c3ccccc3)nc(-n3c4ccccc4c4cc(-c5ccccc5)c5sc6ccccc6c5c43)n2)cc1 ; c1ccc(-c2cc(-c3ccccc3)nc(-n3c4ccc(-n5c6ccccc6c6ccccc65)cc4c4ccc5sc6ccccc6c5c43)n2)cc1]

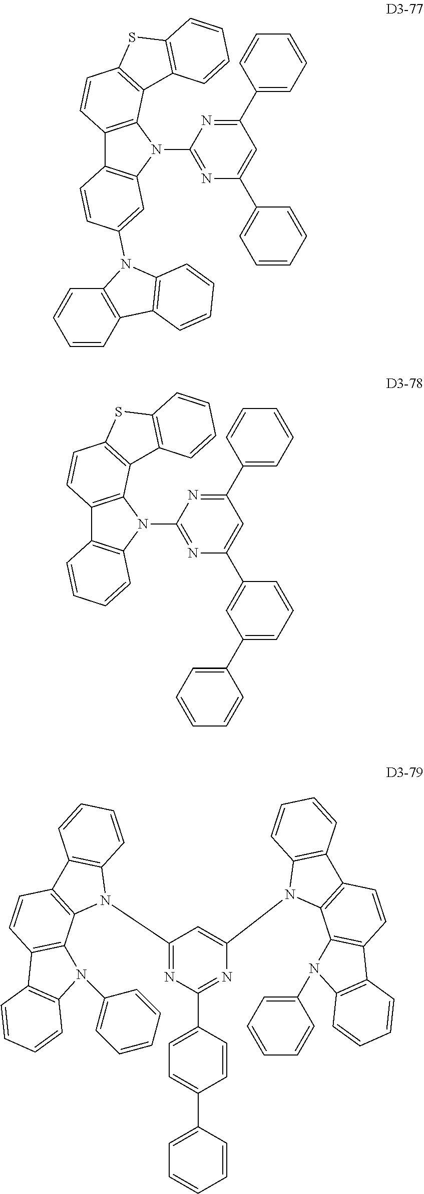 [SMILES: c1ccc(-c2cc(-c3ccccc3)nc(-n3c4cc(-n5c6ccccc6c6ccccc65)ccc4c4ccc5sc6ccccc6c5c43)n2)cc1 ; c1ccc(-c2cccc(-c3cc(-c4ccccc4)nc(-n4c5ccccc5c5ccc6sc7ccccc7c6c54)n3)c2)cc1 ; c1ccc(-c2ccc(-c3nc(-n4c5ccccc5c5ccc6c7ccccc7n(-c7ccccc7)c6c54)cc(-n4c5ccccc5c5ccc6c7ccccc7n(-c7ccccc7)c6c54)n3)cc2)cc1]

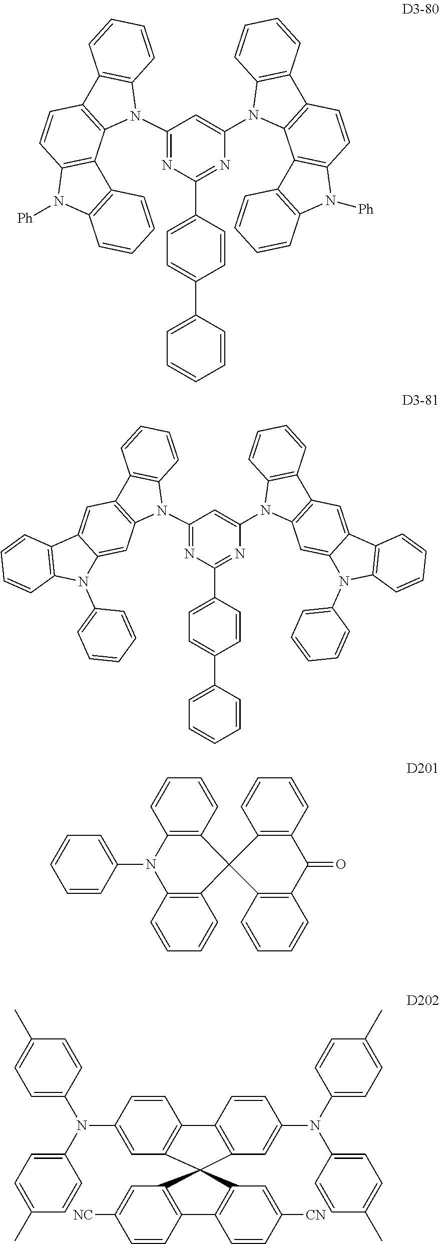

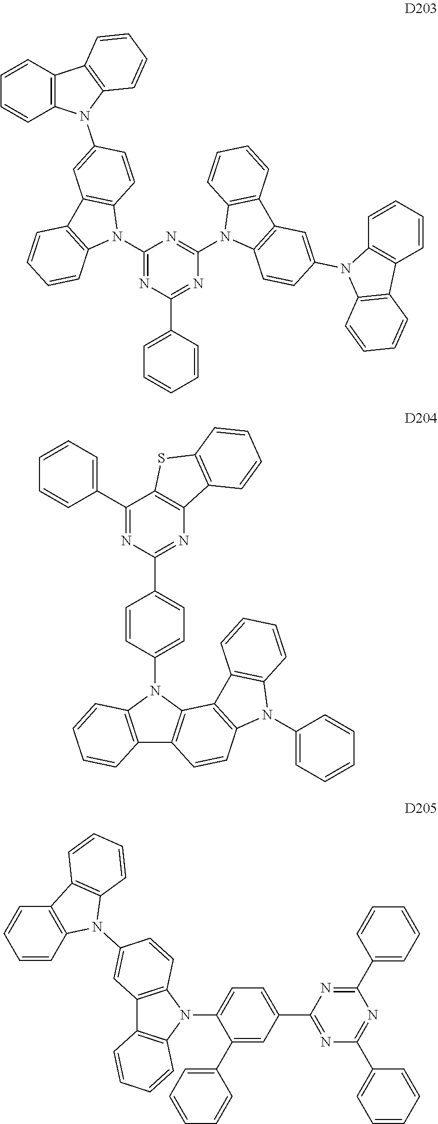 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2nc(-c3ccc(-n4c5ccccc5c5ccc6c(c7ccccc7n6-c6ccccc6)c54)cc3)nc3c2sc2ccccc23)cc1 ; c1ccc(-c2nc(-c3ccccc3)nc(-c3ccc(-n4c5ccccc5c5cc(-n6c7ccccc7c7ccccc76)ccc54)c(-c4ccccc4)c3)n2)cc1]

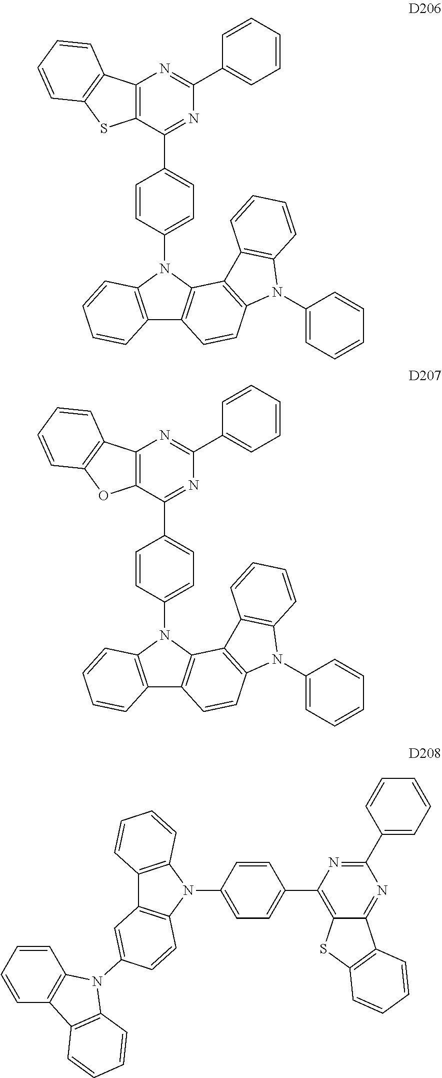 [SMILES: c1ccc(-c2nc(-c3ccc(-n4c5ccccc5c5ccc6c(c7ccccc7n6-c6ccccc6)c54)cc3)c3sc4ccccc4c3n2)cc1 ; c1ccc(-c2nc(-c3ccc(-n4c5ccccc5c5ccc6c(c7ccccc7n6-c6ccccc6)c54)cc3)c3oc4ccccc4c3n2)cc1 ; c1ccc(-c2nc(-c3ccc(-n4c5ccccc5c5cc(-n6c7ccccc7c7ccccc76)ccc54)cc3)c3sc4ccccc4c3n2)cc1]

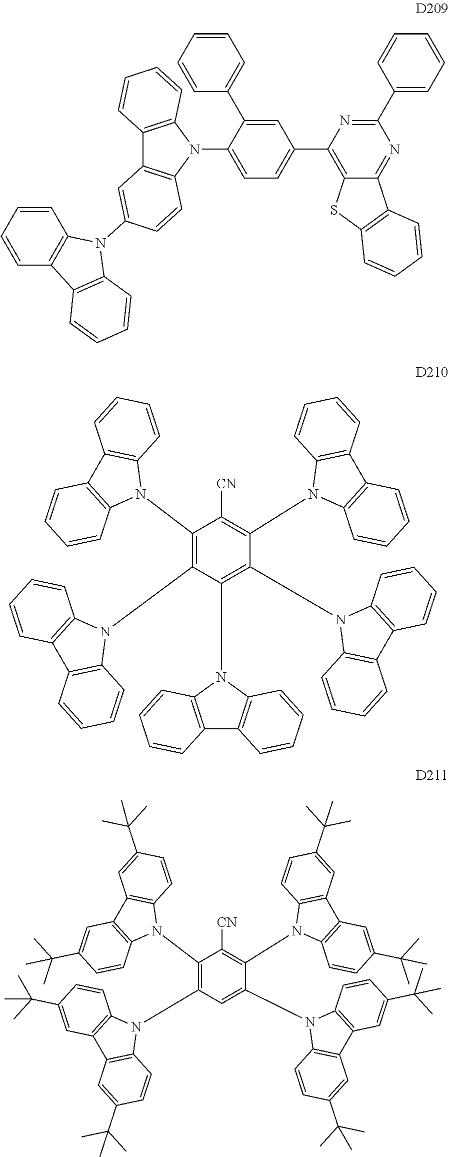 [SMILES: c1ccc(-c2nc(-c3ccc(-n4c5ccccc5c5cc(-n6c7ccccc7c7ccccc76)ccc54)c(-c4ccccc4)c3)c3sc4ccccc4c3n2)cc1 ; N#Cc1c(-n2c3ccccc3c3ccccc32)c(-n2c3ccccc3c3ccccc32)c(-n2c3ccccc3c3ccccc32)c(-n2c3ccccc3c3ccccc32)c1-n1c2ccccc2c2ccccc21 ; CC(C)(C)c1ccc2c(c1)c1cc(C(C)(C)C)ccc1n2-c1cc(-n2c3ccc(C(C)(C)C)cc3c3cc(C(C)(C)C)ccc32)c(-n2c3ccc(C(C)(C)C)cc3c3cc(C(C)(C)C)ccc32)c(C#N)c1-n1c2ccc(C(C)(C)C)cc2c2cc(C(C)(C)C)ccc21]

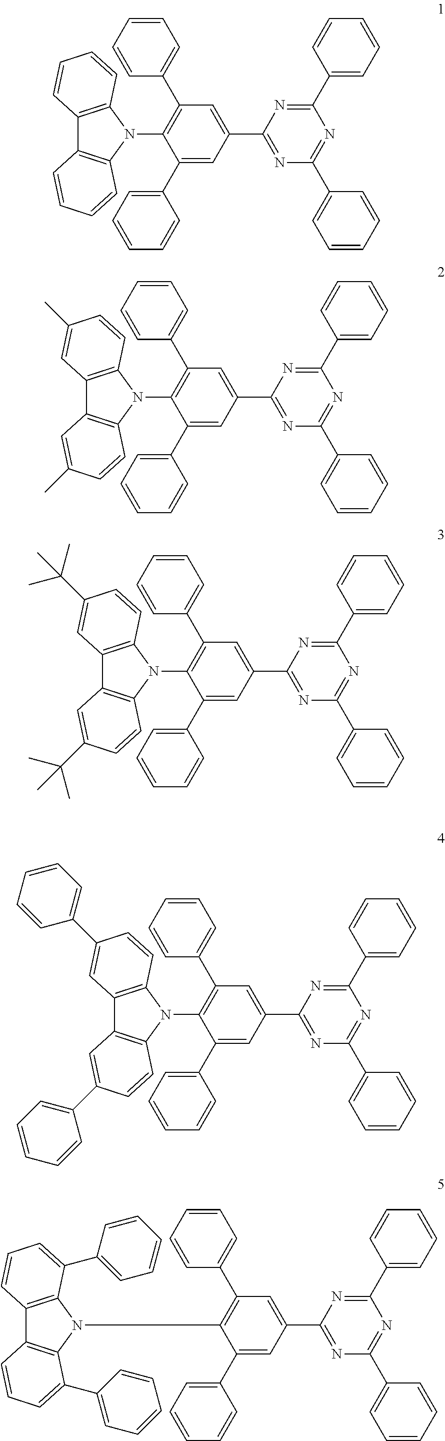

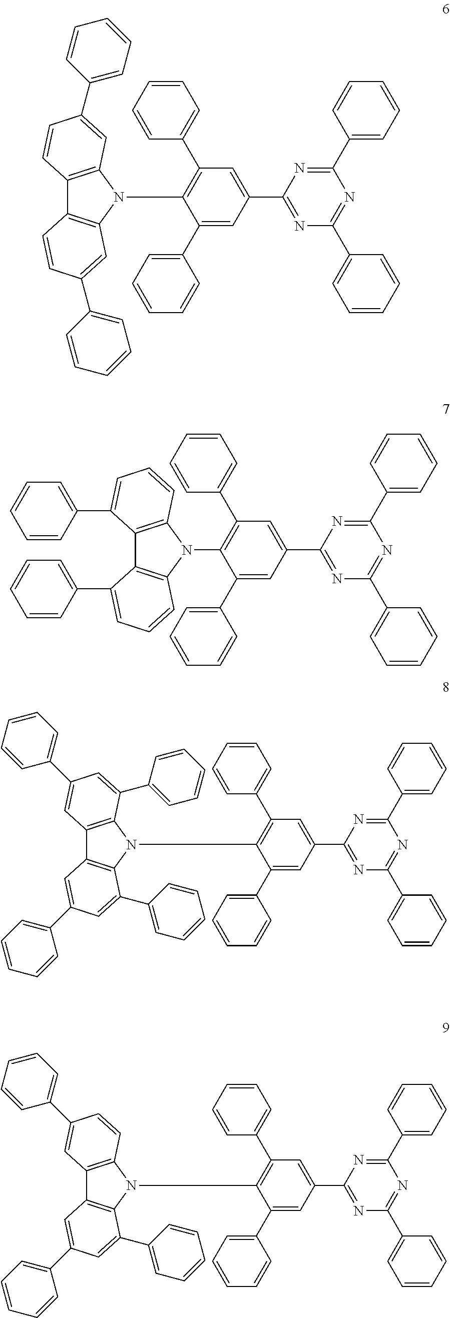 [SMILES: c1ccc(-c2ccc3c4ccc(-c5ccccc5)cc4n(-c4c(-c5ccccc5)cc(-c5nc(-c6ccccc6)nc(-c6ccccc6)n5)cc4-c4ccccc4)c3c2)cc1 ; c1ccc(-c2nc(-c3ccccc3)nc(-c3cc(-c4ccccc4)c(-n4c5cccc(-c6ccccc6)c5c5c(-c6ccccc6)cccc54)c(-c4ccccc4)c3)n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)nc(-c4ccccc4)n3)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccccc4)nc(-c4ccccc4)n3)cc2-c2ccccc2)cc1]

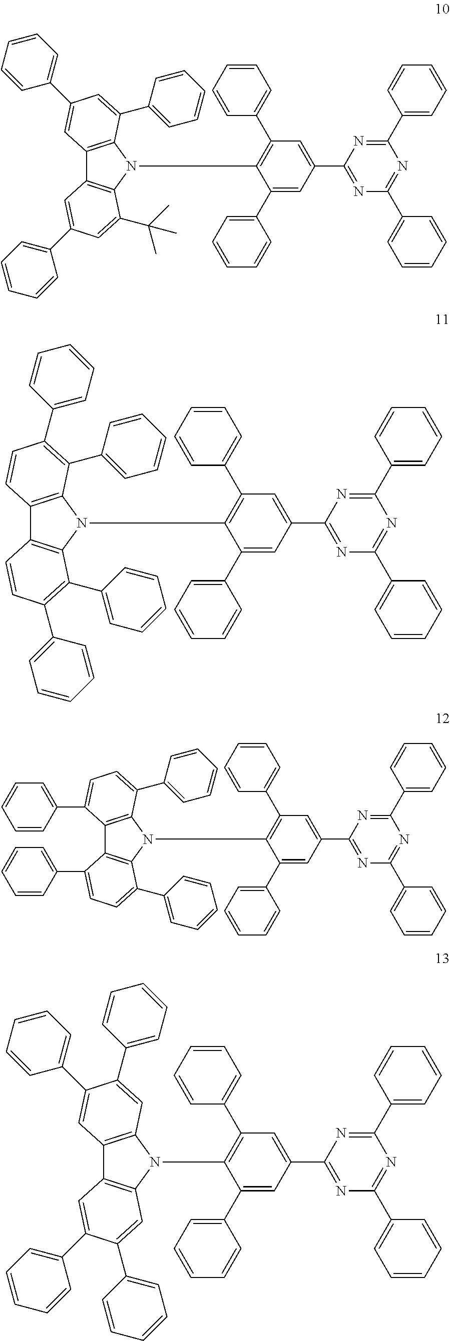

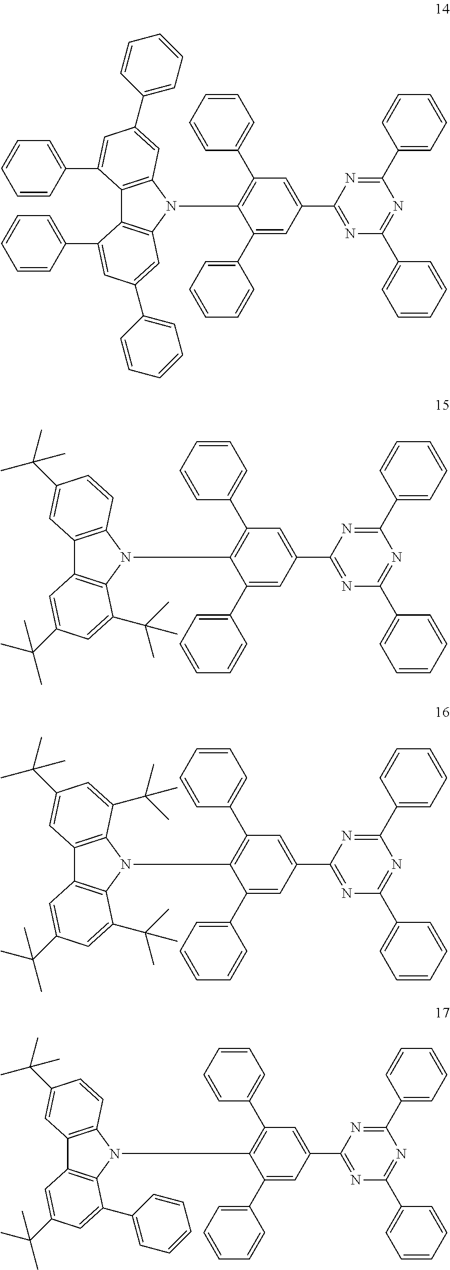 [SMILES: c1ccc(-c2cc(-c3ccccc3)c3c4c(-c5ccccc5)cc(-c5ccccc5)cc4n(-c4c(-c5ccccc5)cc(-c5nc(-c6ccccc6)nc(-c6ccccc6)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)nc(-c3ccccc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)nc(-c3ccccc3)n2)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccccc3)nc(-c3ccccc3)n2)cc1-c1ccccc1]

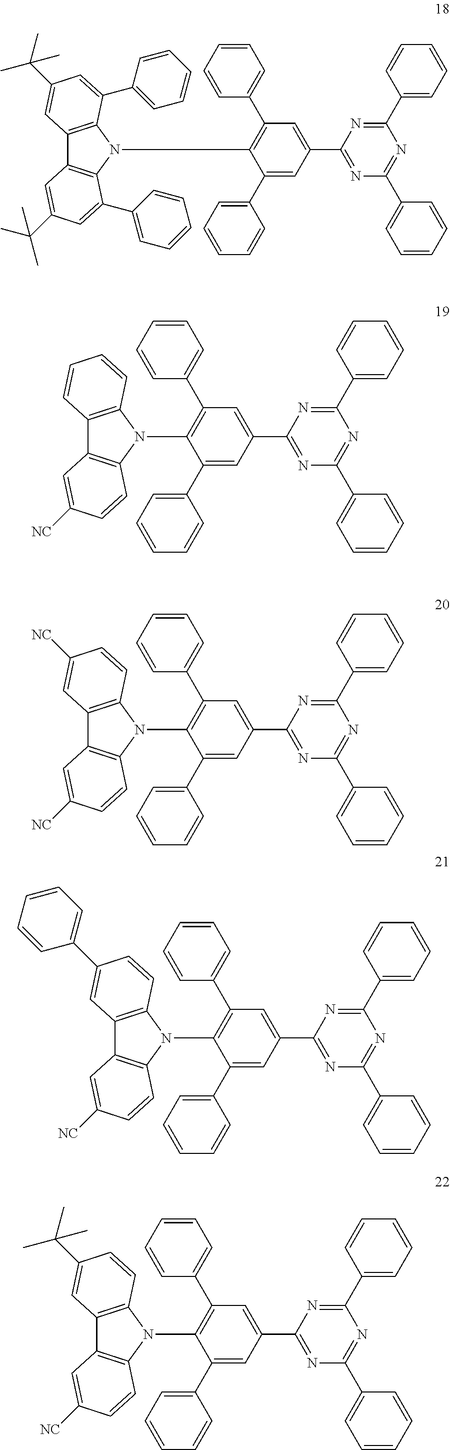

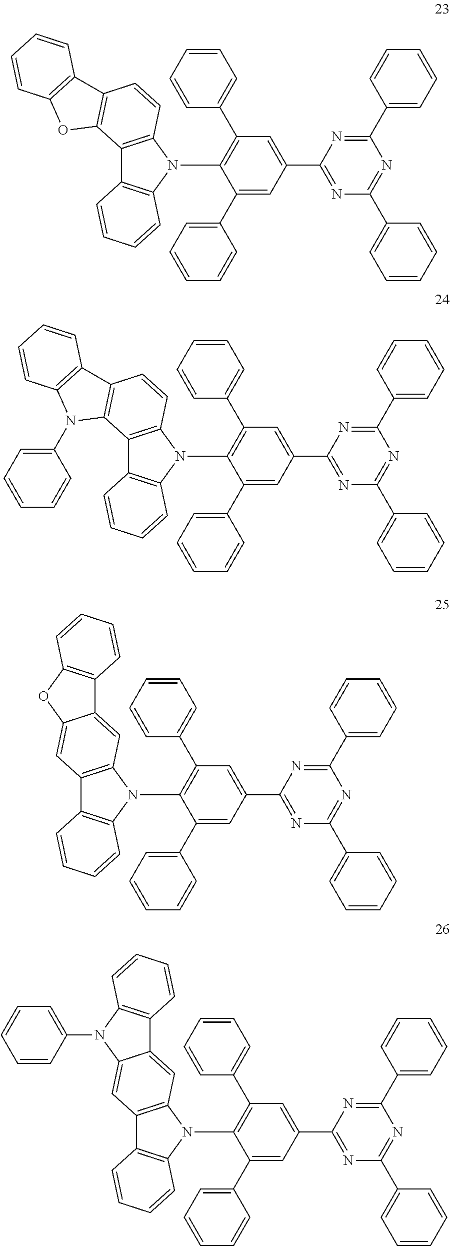

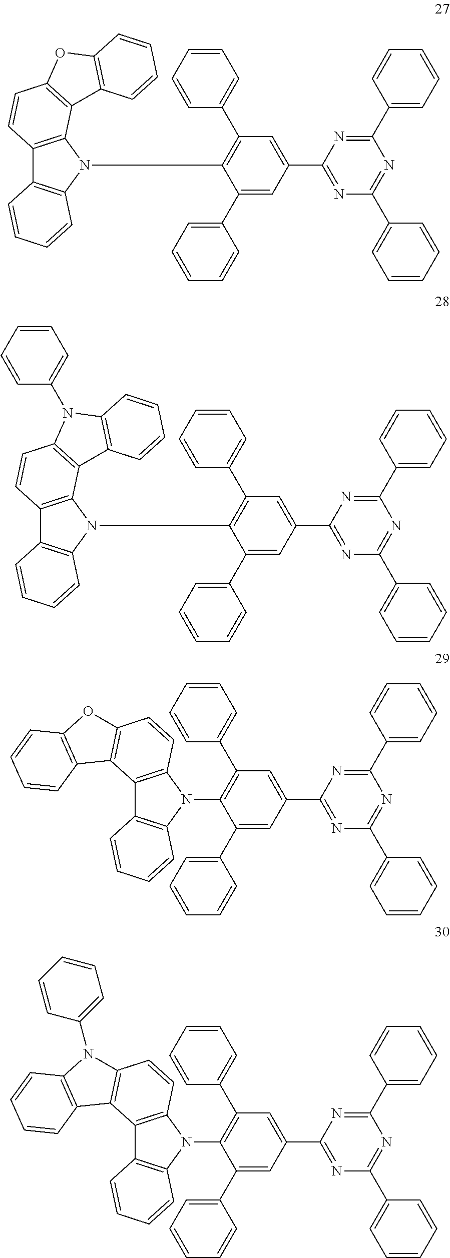

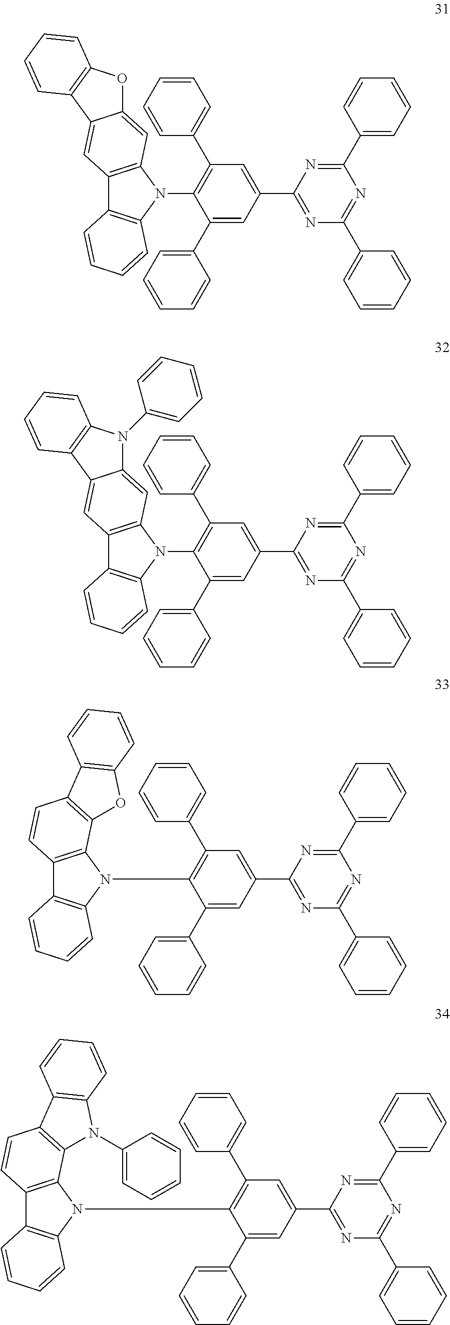 [SMILES: c1ccc(-c2nc(-c3ccccc3)nc(-c3cc(-c4ccccc4)c(-n4c5ccccc5c5cc6c(cc54)oc4ccccc46)c(-c4ccccc4)c3)n2)cc1 ; c1ccc(-c2nc(-c3ccccc3)nc(-c3cc(-c4ccccc4)c(-n4c5ccccc5c5cc6c7ccccc7n(-c7ccccc7)c6cc54)c(-c4ccccc4)c3)n2)cc1 ; c1ccc(-c2nc(-c3ccccc3)nc(-c3cc(-c4ccccc4)c(-n4c5ccccc5c5ccc6c7ccccc7oc6c54)c(-c4ccccc4)c3)n2)cc1 ; c1ccc(-c2nc(-c3ccccc3)nc(-c3cc(-c4ccccc4)c(-n4c5ccccc5c5ccc6c7ccccc7n(-c7ccccc7)c6c54)c(-c4ccccc4)c3)n2)cc1]

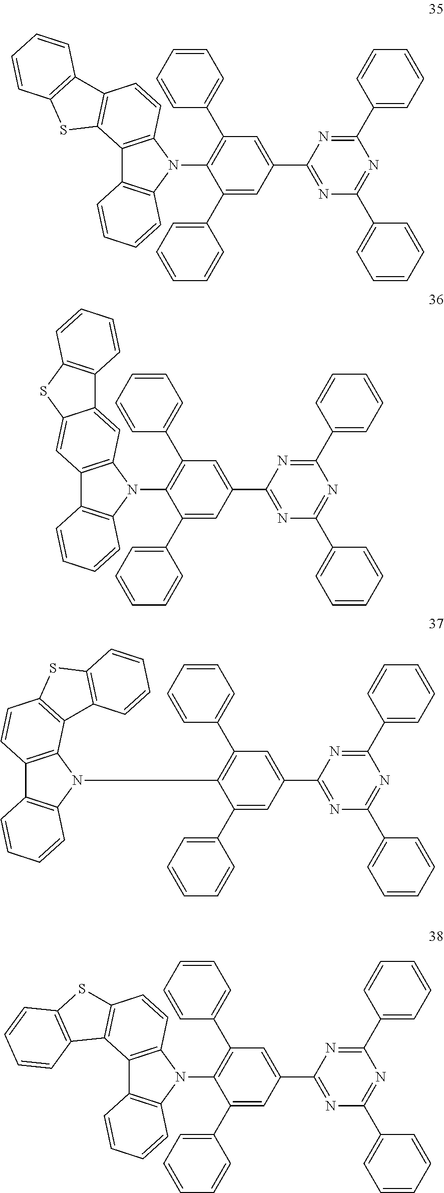 [SMILES: c1ccc(-c2nc(-c3ccccc3)nc(-c3cc(-c4ccccc4)c(-n4c5ccccc5c5c6sc7ccccc7c6ccc54)c(-c4ccccc4)c3)n2)cc1 ; c1ccc(-c2nc(-c3ccccc3)nc(-c3cc(-c4ccccc4)c(-n4c5ccccc5c5cc6sc7ccccc7c6cc54)c(-c4ccccc4)c3)n2)cc1 ; c1ccc(-c2nc(-c3ccccc3)nc(-c3cc(-c4ccccc4)c(-n4c5ccccc5c5ccc6sc7ccccc7c6c54)c(-c4ccccc4)c3)n2)cc1 ; c1ccc(-c2nc(-c3ccccc3)nc(-c3cc(-c4ccccc4)c(-n4c5ccccc5c5c6c(ccc54)sc4ccccc46)c(-c4ccccc4)c3)n2)cc1]

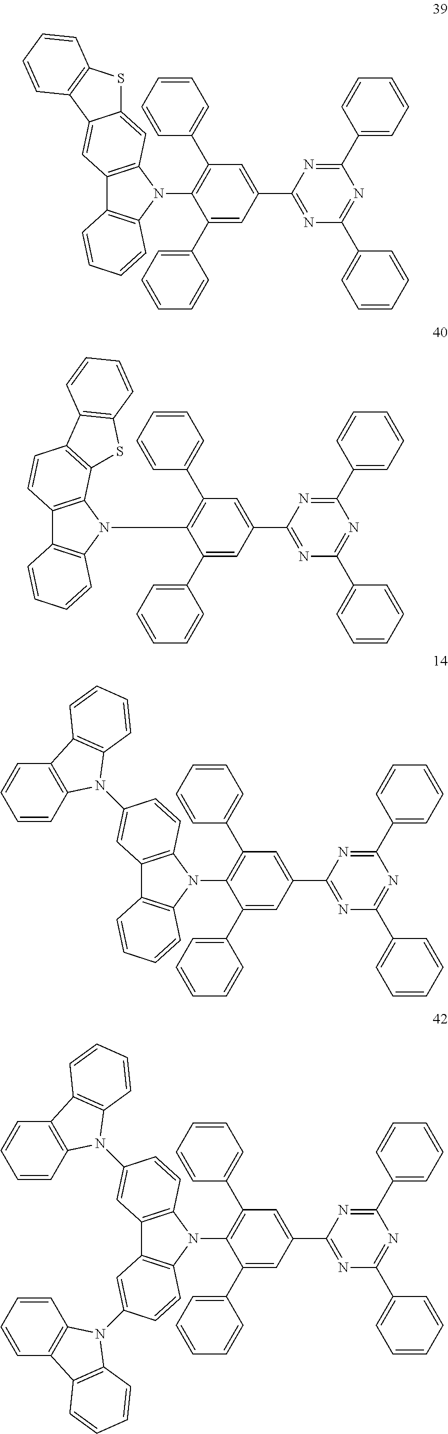 [SMILES: c1ccc(-c2nc(-c3ccccc3)nc(-c3cc(-c4ccccc4)c(-n4c5ccccc5c5cc6c(cc54)sc4ccccc46)c(-c4ccccc4)c3)n2)cc1 ; c1ccc(-c2nc(-c3ccccc3)nc(-c3cc(-c4ccccc4)c(-n4c5ccccc5c5ccc6c7ccccc7sc6c54)c(-c4ccccc4)c3)n2)cc1 ; c1ccc(-c2nc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; c1ccc(-c2nc(-c3ccccc3)nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)n2)cc1]

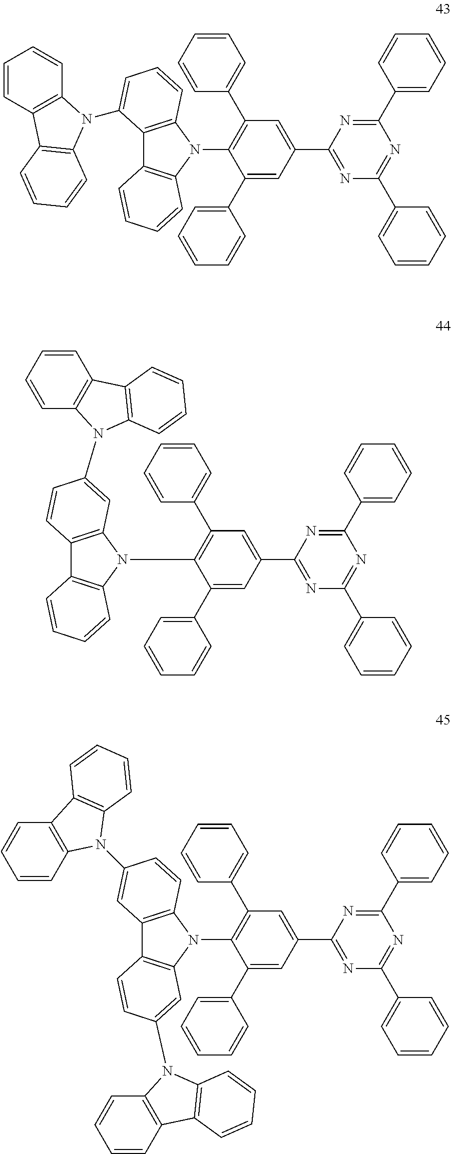 [SMILES: c1ccc(-c2nc(-c3ccccc3)nc(-c3cc(-c4ccccc4)c(-n4c5ccccc5c5c(-n6c7ccccc7c7ccccc76)cccc54)c(-c4ccccc4)c3)n2)cc1 ; c1ccc(-c2nc(-c3ccccc3)nc(-c3cc(-c4ccccc4)c(-n4c5ccccc5c5ccc(-n6c7ccccc7c7ccccc76)cc54)c(-c4ccccc4)c3)n2)cc1 ; c1ccc(-c2nc(-c3ccccc3)nc(-c3cc(-c4ccccc4)c(-n4c5ccc(-n6c7ccccc7c7ccccc76)cc5c5ccc(-n6c7ccccc7c7ccccc76)cc54)c(-c4ccccc4)c3)n2)cc1]

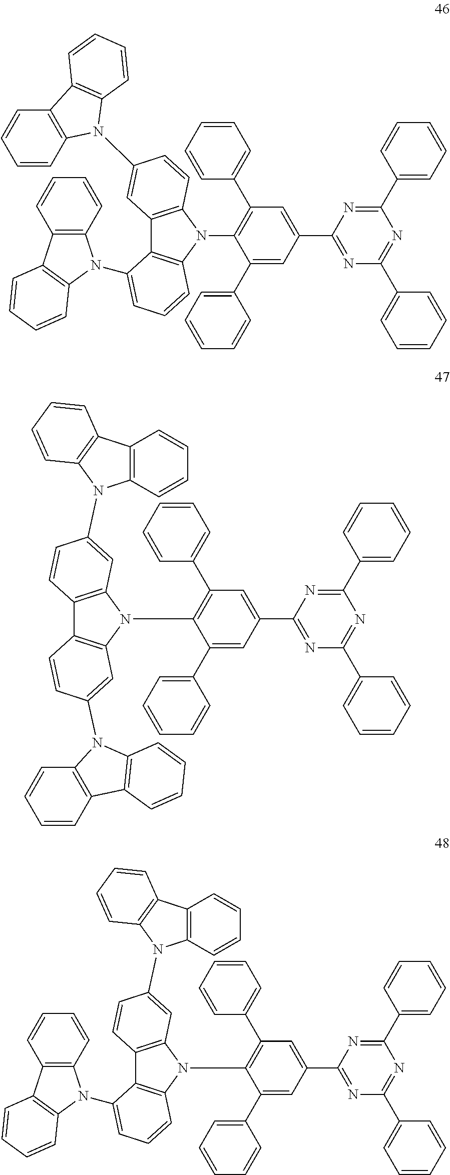 [SMILES: c1ccc(-c2nc(-c3ccccc3)nc(-c3cc(-c4ccccc4)c(-n4c5ccc(-n6c7ccccc7c7ccccc76)cc5c5c(-n6c7ccccc7c7ccccc76)cccc54)c(-c4ccccc4)c3)n2)cc1 ; c1ccc(-c2nc(-c3ccccc3)nc(-c3cc(-c4ccccc4)c(-n4c5cc(-n6c7ccccc7c7ccccc76)ccc5c5ccc(-n6c7ccccc7c7ccccc76)cc54)c(-c4ccccc4)c3)n2)cc1 ; c1ccc(-c2nc(-c3ccccc3)nc(-c3cc(-c4ccccc4)c(-n4c5cc(-n6c7ccccc7c7ccccc76)ccc5c5c(-n6c7ccccc7c7ccccc76)cccc54)c(-c4ccccc4)c3)n2)cc1]

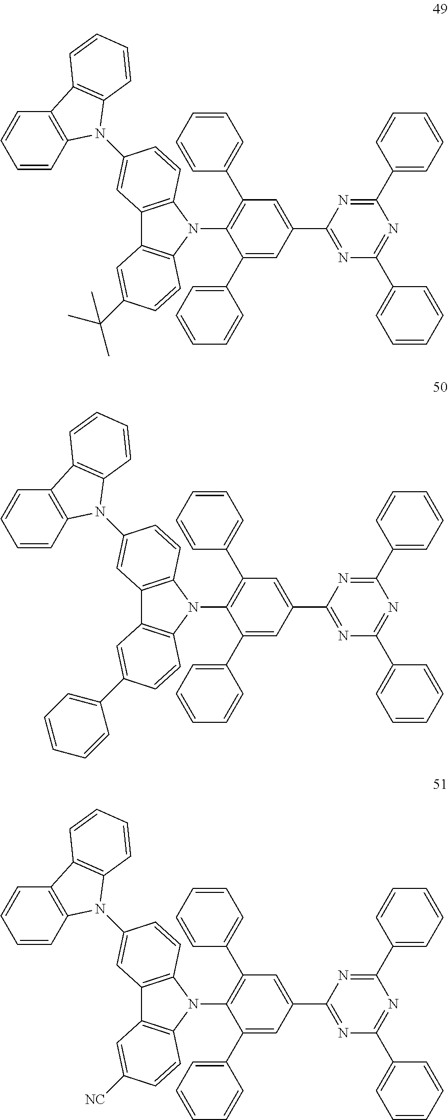 [SMILES: CC(C)(C)c1ccc2c(c1)c1cc(-n3c4ccccc4c4ccccc43)ccc1n2-c1c(-c2ccccc2)cc(-c2nc(-c3ccccc3)nc(-c3ccccc3)n2)cc1-c1ccccc1 ; c1ccc(-c2ccc3c(c2)c2cc(-n4c5ccccc5c5ccccc54)ccc2n3-c2c(-c3ccccc3)cc(-c3nc(-c4ccccc4)nc(-c4ccccc4)n3)cc2-c2ccccc2)cc1 ; N#Cc1ccc2c(c1)c1cc(-n3c4ccccc4c4ccccc43)ccc1n2-c1c(-c2ccccc2)cc(-c2nc(-c3ccccc3)nc(-c3ccccc3)n2)cc1-c1ccccc1]

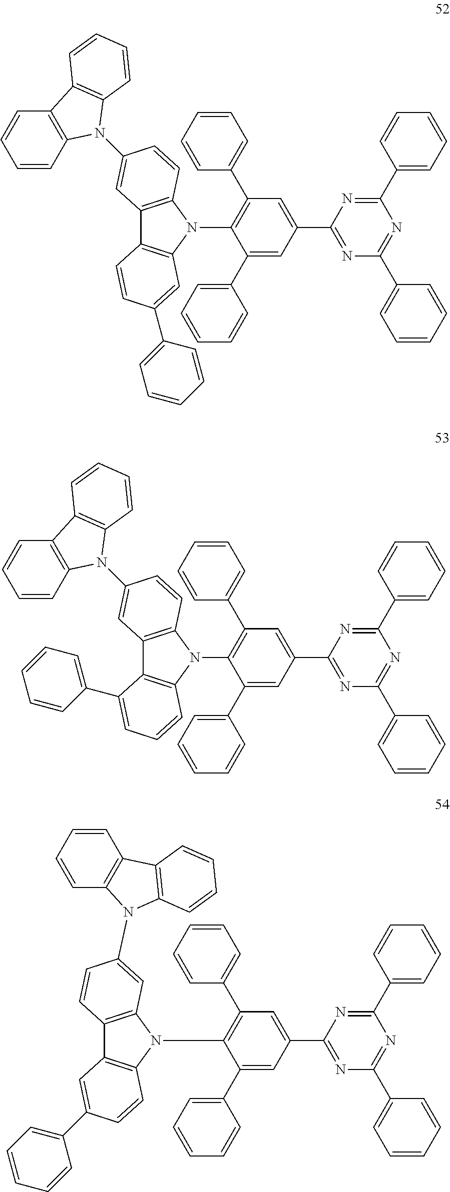 [SMILES: c1ccc(-c2ccc3c4cc(-n5c6ccccc6c6ccccc65)ccc4n(-c4c(-c5ccccc5)cc(-c5nc(-c6ccccc6)nc(-c6ccccc6)n5)cc4-c4ccccc4)c3c2)cc1 ; c1ccc(-c2nc(-c3ccccc3)nc(-c3cc(-c4ccccc4)c(-n4c5ccc(-n6c7ccccc7c7ccccc76)cc5c5c(-c6ccccc6)cccc54)c(-c4ccccc4)c3)n2)cc1 ; c1ccc(-c2ccc3c(c2)c2ccc(-n4c5ccccc5c5ccccc54)cc2n3-c2c(-c3ccccc3)cc(-c3nc(-c4ccccc4)nc(-c4ccccc4)n3)cc2-c2ccccc2)cc1]

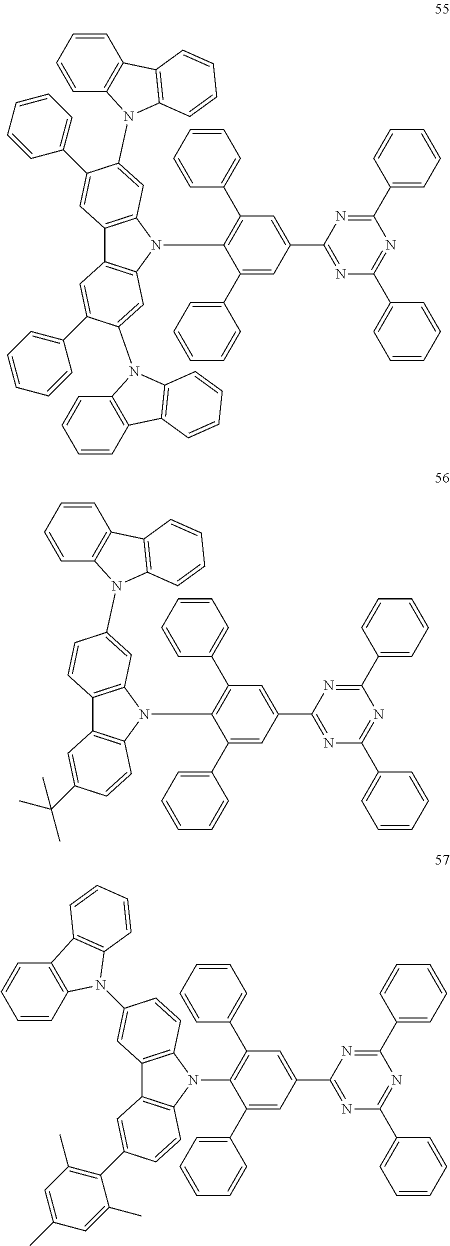 [SMILES: c1ccc(-c2nc(-c3ccccc3)nc(-c3cc(-c4ccccc4)c(-n4c5cc(-n6c7ccccc7c7ccccc76)c(-c6ccccc6)cc5c5cc(-c6ccccc6)c(-n6c7ccccc7c7ccccc76)cc54)c(-c4ccccc4)c3)n2)cc1 ; CC(C)(C)c1ccc2c(c1)c1ccc(-n3c4ccccc4c4ccccc43)cc1n2-c1c(-c2ccccc2)cc(-c2nc(-c3ccccc3)nc(-c3ccccc3)n2)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccccc4)nc(-c4ccccc4)n3)cc2-c2ccccc2)c(C)c1]

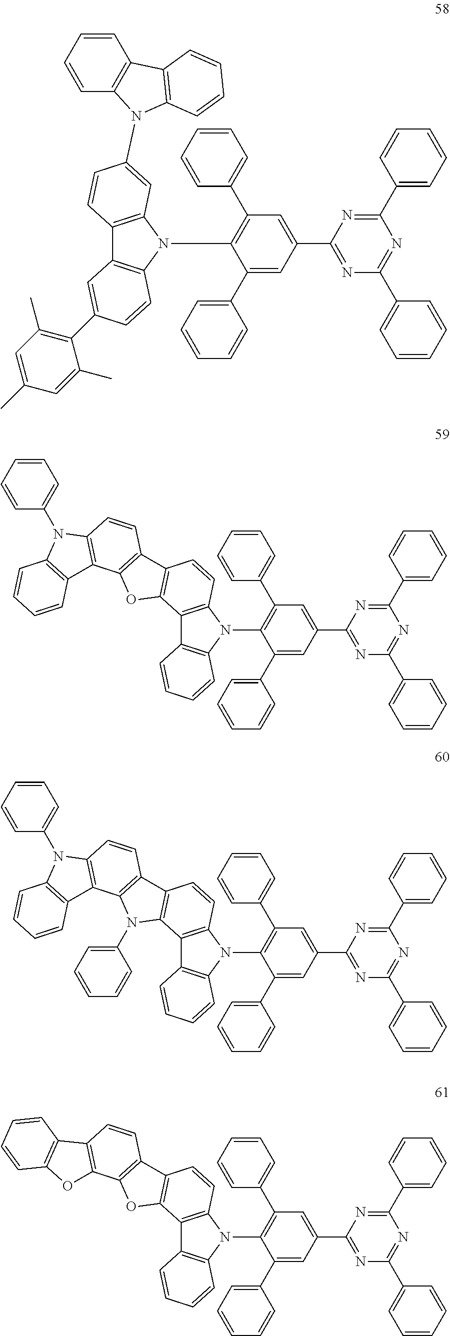 [SMILES: Cc1cc(C)c(-c2ccc3c(c2)c2ccc(-n4c5ccccc5c5ccccc54)cc2n3-c2c(-c3ccccc3)cc(-c3nc(-c4ccccc4)nc(-c4ccccc4)n3)cc2-c2ccccc2)c(C)c1 ; c1ccc(-c2nc(-c3ccccc3)nc(-c3cc(-c4ccccc4)c(-n4c5ccccc5c5c6oc7c(ccc8c7c7ccccc7n8-c7ccccc7)c6ccc54)c(-c4ccccc4)c3)n2)cc1 ; c1ccc(-c2nc(-c3ccccc3)nc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)n2)cc1 ; c1ccc(-c2nc(-c3ccccc3)nc(-c3cc(-c4ccccc4)c(-n4c5ccccc5c5c6oc7c(ccc8c9ccccc9oc87)c6ccc54)c(-c4ccccc4)c3)n2)cc1]

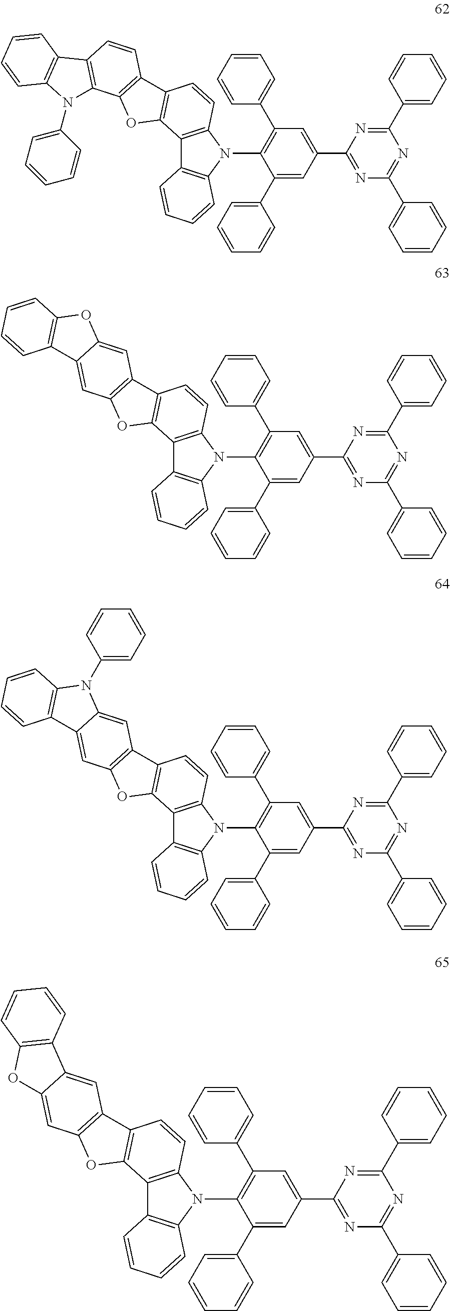 [SMILES: c1ccc(-c2nc(-c3ccccc3)nc(-c3cc(-c4ccccc4)c(-n4c5ccccc5c5c6oc7c(ccc8c9ccccc9n(-c9ccccc9)c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7cc8c(cc7c6ccc54)oc4ccccc48)c(-c4ccccc4)c3)n2)cc1 ; c1ccc(-c2nc(-c3ccccc3)nc(-c3cc(-c4ccccc4)c(-n4c5ccccc5c5c6oc7cc8c9ccccc9n(-c9ccccc9)c8cc7c6ccc54)c(-c4ccccc4)c3)n2)cc1 ; c1ccc(-c2nc(-c3ccccc3)nc(-c3cc(-c4ccccc4)c(-n4c5ccccc5c5c6oc7cc8oc9ccccc9c8cc7c6ccc54)c(-c4ccccc4)c3)n2)cc1]

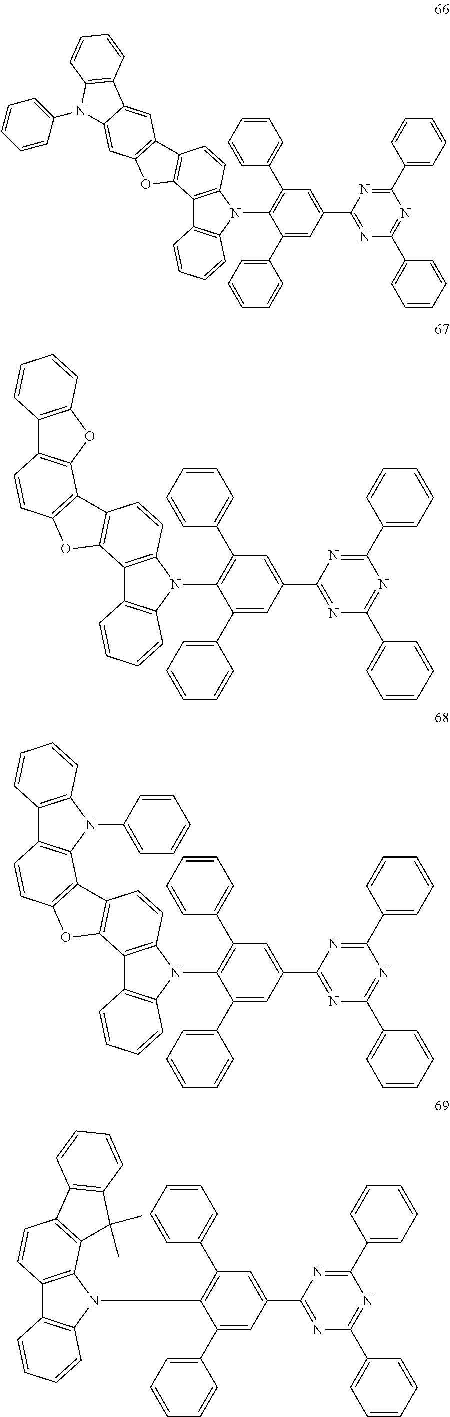 [SMILES: c1ccc(-c2nc(-c3ccccc3)nc(-c3cc(-c4ccccc4)c(-n4c5ccccc5c5c6oc7cc8c(cc7c6ccc54)c4ccccc4n8-c4ccccc4)c(-c4ccccc4)c3)n2)cc1 ; c1ccc(-c2nc(-c3ccccc3)nc(-c3cc(-c4ccccc4)c(-n4c5ccccc5c5c6oc7ccc8c9ccccc9oc8c7c6ccc54)c(-c4ccccc4)c3)n2)cc1 ; c1ccc(-c2nc(-c3ccccc3)nc(-c3cc(-c4ccccc4)c(-n4c5ccccc5c5c6oc7ccc8c9ccccc9n(-c9ccccc9)c8c7c6ccc54)c(-c4ccccc4)c3)n2)cc1 ; CC1(C)c2ccccc2-c2ccc3c4ccccc4n(-c4c(-c5ccccc5)cc(-c5nc(-c6ccccc6)nc(-c6ccccc6)n5)cc4-c4ccccc4)c3c21]

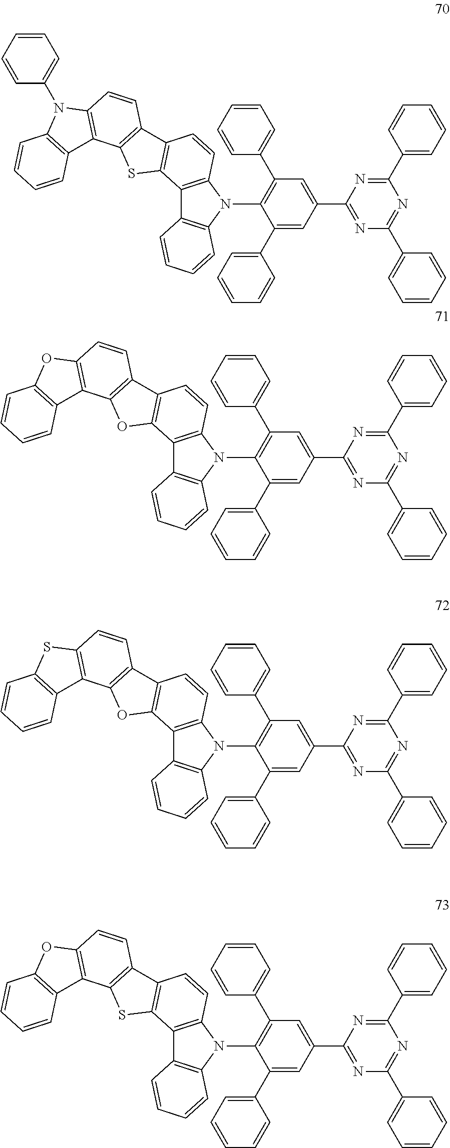 [SMILES: c1ccc(-c2nc(-c3ccccc3)nc(-c3cc(-c4ccccc4)c(-n4c5ccccc5c5c6sc7c(ccc8c7c7ccccc7n8-c7ccccc7)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(ccc8oc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(ccc8sc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6sc7c(ccc8oc9ccccc9c87)c6ccc54)c(-c4ccccc4)c3)n2)cc1]

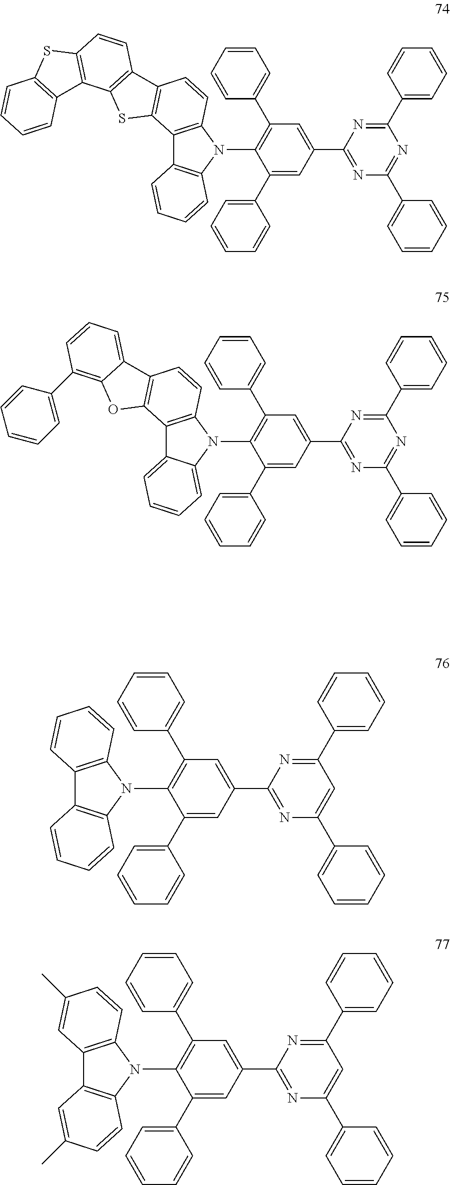 [SMILES: c1ccc(-c2nc(-c3ccccc3)nc(-c3cc(-c4ccccc4)c(-n4c5ccccc5c5c6sc7c(ccc8sc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(-c8ccccc8)cccc7c6ccc54)c(-c4ccccc4)c3)n2)cc1 ; c1ccc(-c2cc(-c3ccccc3)nc(-c3cc(-c4ccccc4)c(-n4c5ccccc5c5ccccc54)c(-c4ccccc4)c3)n2)cc1 ; Cc1ccc2c(c1)c1cc(C)ccc1n2-c1c(-c2ccccc2)cc(-c2nc(-c3ccccc3)cc(-c3ccccc3)n2)cc1-c1ccccc1]

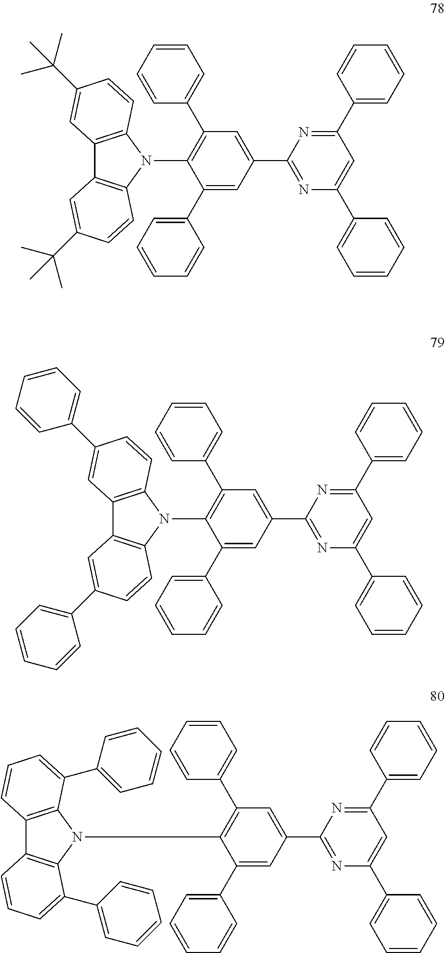 [SMILES: CC(C)(C)c1ccc2c(c1)c1cc(C(C)(C)C)ccc1n2-c1c(-c2ccccc2)cc(-c2nc(-c3ccccc3)cc(-c3ccccc3)n2)cc1-c1ccccc1 ; c1ccc(-c2ccc3c(c2)c2cc(-c4ccccc4)ccc2n3-c2c(-c3ccccc3)cc(-c3nc(-c4ccccc4)cc(-c4ccccc4)n3)cc2-c2ccccc2)cc1 ; c1ccc(-c2cc(-c3ccccc3)nc(-c3cc(-c4ccccc4)c(-n4c5c(-c6ccccc6)cccc5c5cccc(-c6ccccc6)c54)c(-c4ccccc4)c3)n2)cc1]

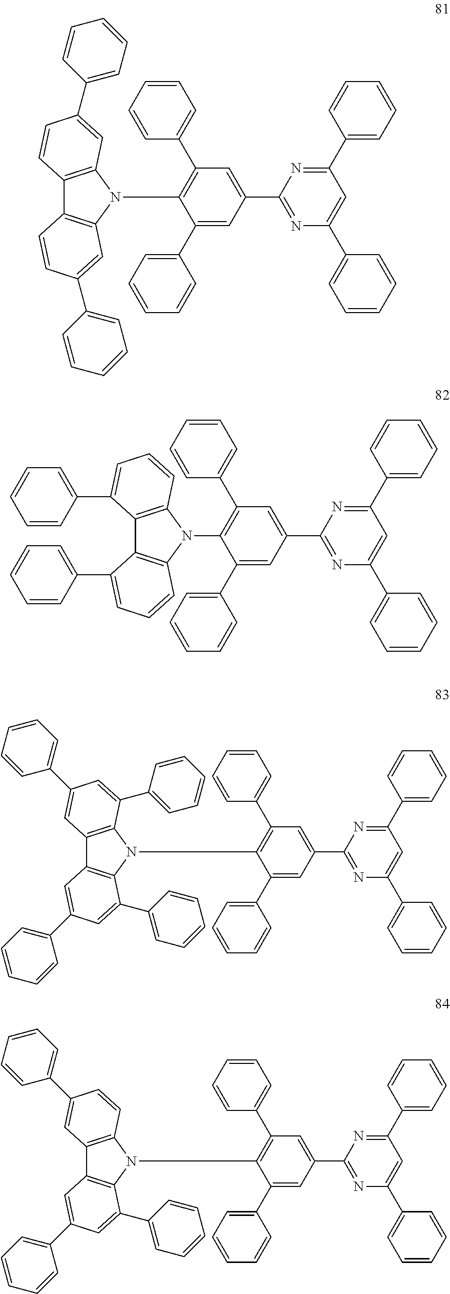 [SMILES: c1ccc(-c2ccc3c4ccc(-c5ccccc5)cc4n(-c4c(-c5ccccc5)cc(-c5nc(-c6ccccc6)cc(-c6ccccc6)n5)cc4-c4ccccc4)c3c2)cc1 ; c1ccc(-c2cc(-c3ccccc3)nc(-c3cc(-c4ccccc4)c(-n4c5cccc(-c6ccccc6)c5c5c(-c6ccccc6)cccc54)c(-c4ccccc4)c3)n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)cc(-c4ccccc4)n3)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccccc4)cc(-c4ccccc4)n3)cc2-c2ccccc2)cc1]

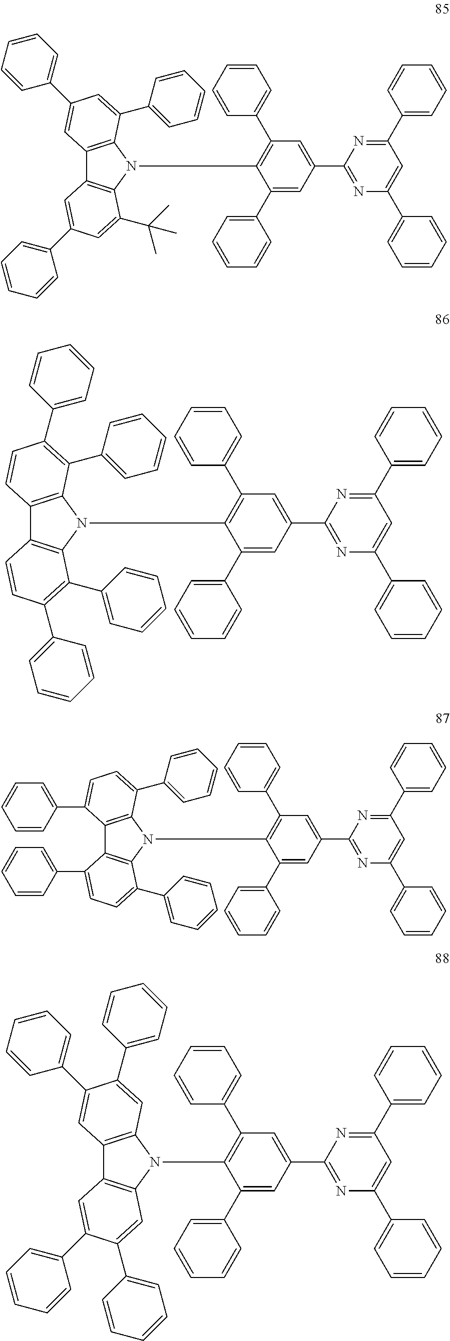

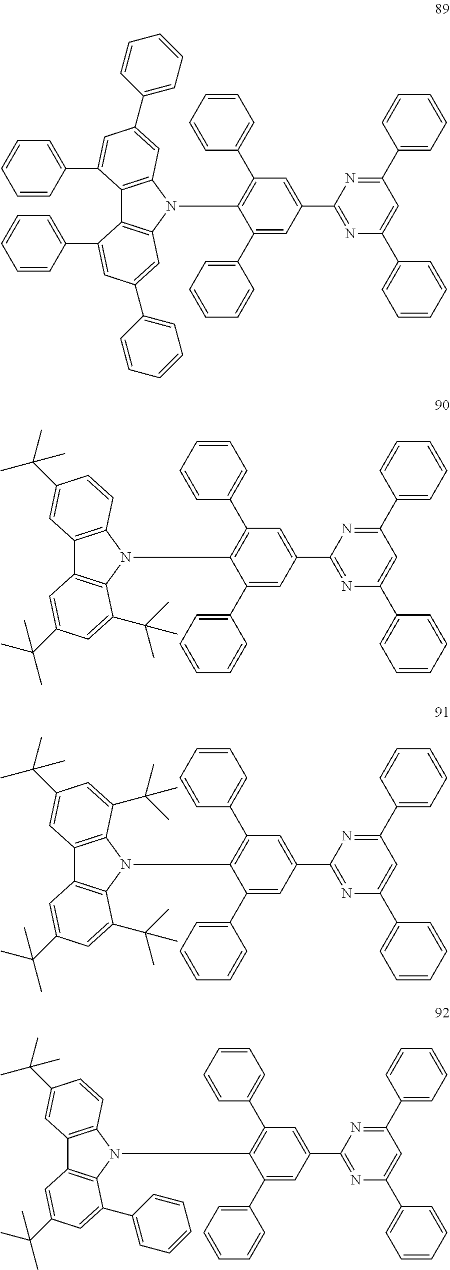 [SMILES: c1ccc(-c2cc(-c3ccccc3)c3c4c(-c5ccccc5)cc(-c5ccccc5)cc4n(-c4c(-c5ccccc5)cc(-c5nc(-c6ccccc6)cc(-c6ccccc6)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)cc(-c3ccccc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)cc(-c3ccccc3)n2)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccccc3)cc(-c3ccccc3)n2)cc1-c1ccccc1]

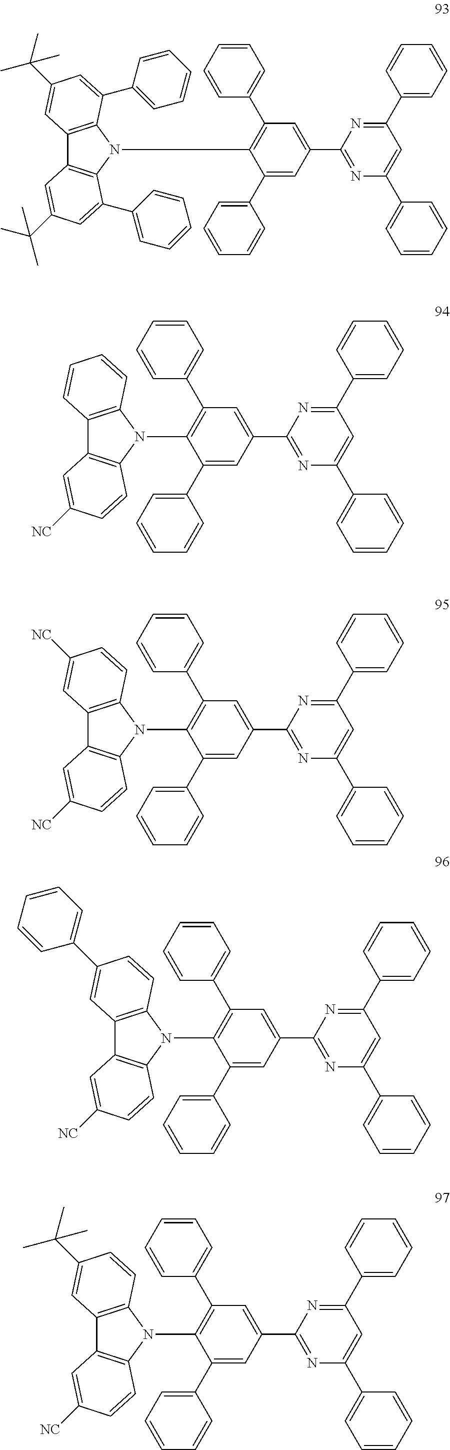

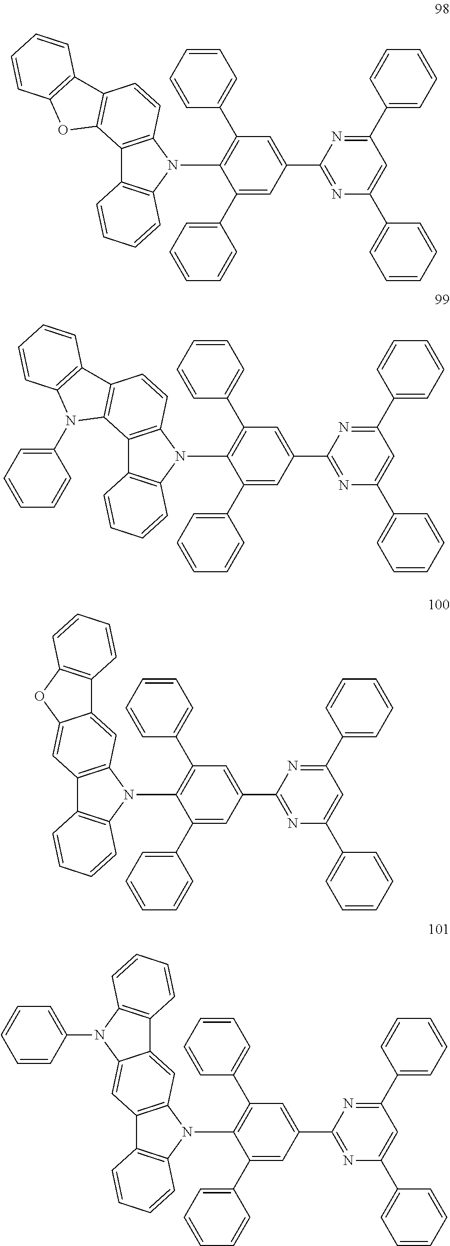

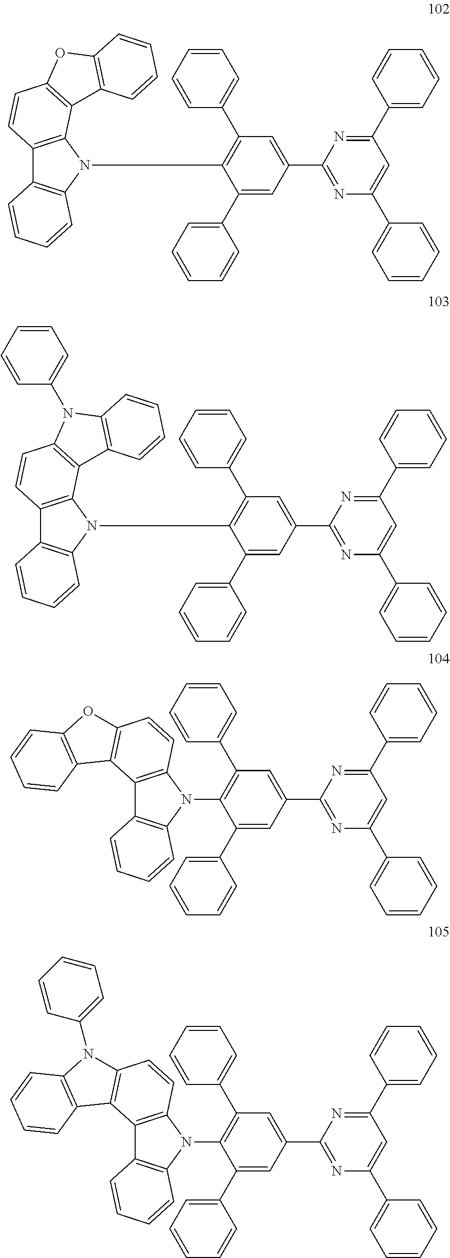

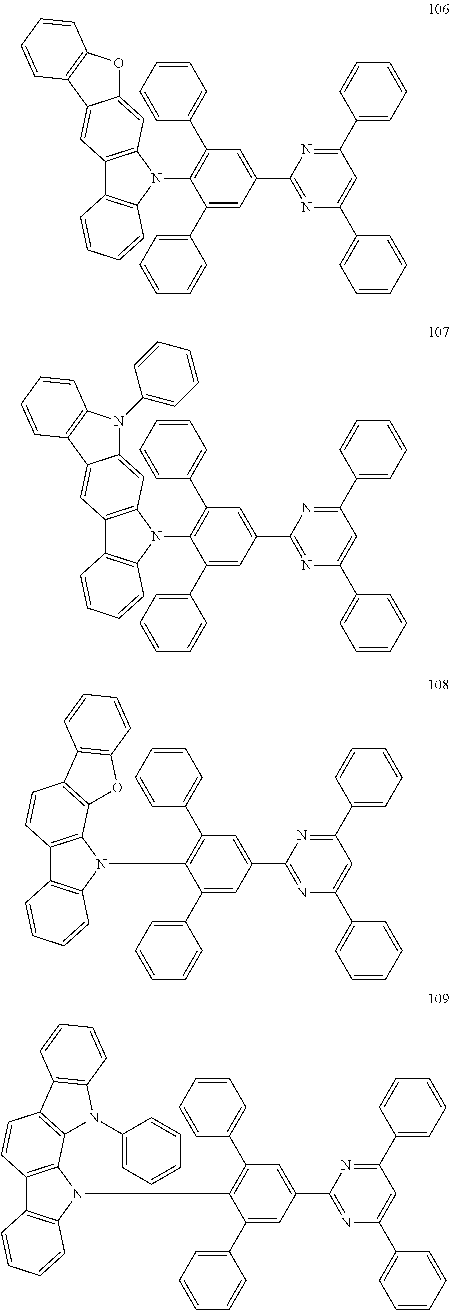 [SMILES: c1ccc(-c2cc(-c3ccccc3)nc(-c3cc(-c4ccccc4)c(-n4c5ccccc5c5cc6c(cc54)oc4ccccc46)c(-c4ccccc4)c3)n2)cc1 ; c1ccc(-c2cc(-c3ccccc3)nc(-c3cc(-c4ccccc4)c(-n4c5ccccc5c5cc6c7ccccc7n(-c7ccccc7)c6cc54)c(-c4ccccc4)c3)n2)cc1 ; c1ccc(-c2cc(-c3ccccc3)nc(-c3cc(-c4ccccc4)c(-n4c5ccccc5c5ccc6c7ccccc7oc6c54)c(-c4ccccc4)c3)n2)cc1 ; c1ccc(-c2cc(-c3ccccc3)nc(-c3cc(-c4ccccc4)c(-n4c5ccccc5c5ccc6c7ccccc7n(-c7ccccc7)c6c54)c(-c4ccccc4)c3)n2)cc1]

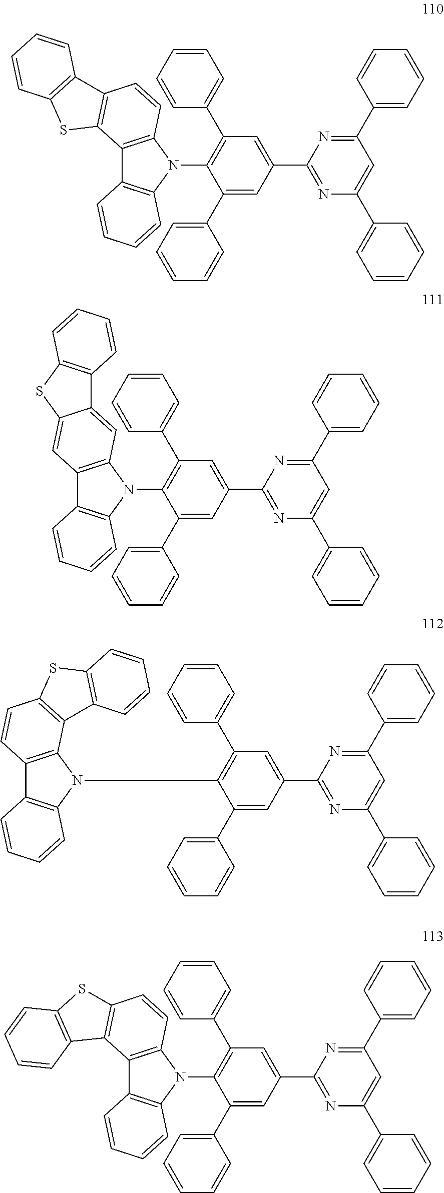 [SMILES: c1ccc(-c2cc(-c3ccccc3)nc(-c3cc(-c4ccccc4)c(-n4c5ccccc5c5c6sc7ccccc7c6ccc54)c(-c4ccccc4)c3)n2)cc1 ; c1ccc(-c2cc(-c3ccccc3)nc(-c3cc(-c4ccccc4)c(-n4c5ccccc5c5cc6sc7ccccc7c6cc54)c(-c4ccccc4)c3)n2)cc1 ; c1ccc(-c2cc(-c3ccccc3)nc(-c3cc(-c4ccccc4)c(-n4c5ccccc5c5ccc6sc7ccccc7c6c54)c(-c4ccccc4)c3)n2)cc1 ; c1ccc(-c2cc(-c3ccccc3)nc(-c3cc(-c4ccccc4)c(-n4c5ccccc5c5c6c(ccc54)sc4ccccc46)c(-c4ccccc4)c3)n2)cc1]

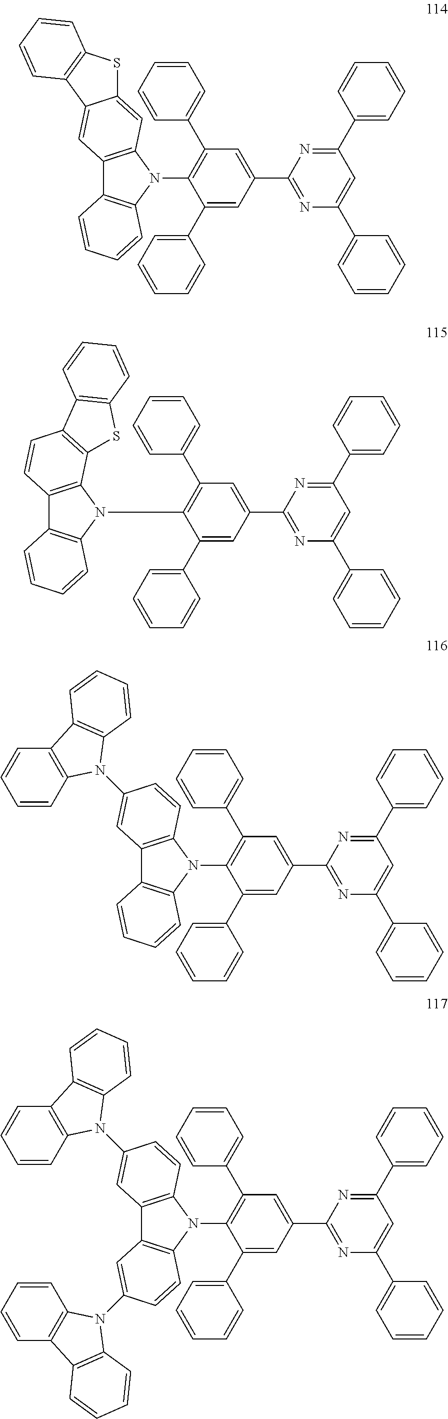 [SMILES: c1ccc(-c2cc(-c3ccccc3)nc(-c3cc(-c4ccccc4)c(-n4c5ccccc5c5cc6c(cc54)sc4ccccc46)c(-c4ccccc4)c3)n2)cc1 ; c1ccc(-c2cc(-c3ccccc3)nc(-c3cc(-c4ccccc4)c(-n4c5ccccc5c5ccc6c7ccccc7sc6c54)c(-c4ccccc4)c3)n2)cc1 ; c1ccc(-c2cc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; c1ccc(-c2cc(-c3ccccc3)nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)n2)cc1]

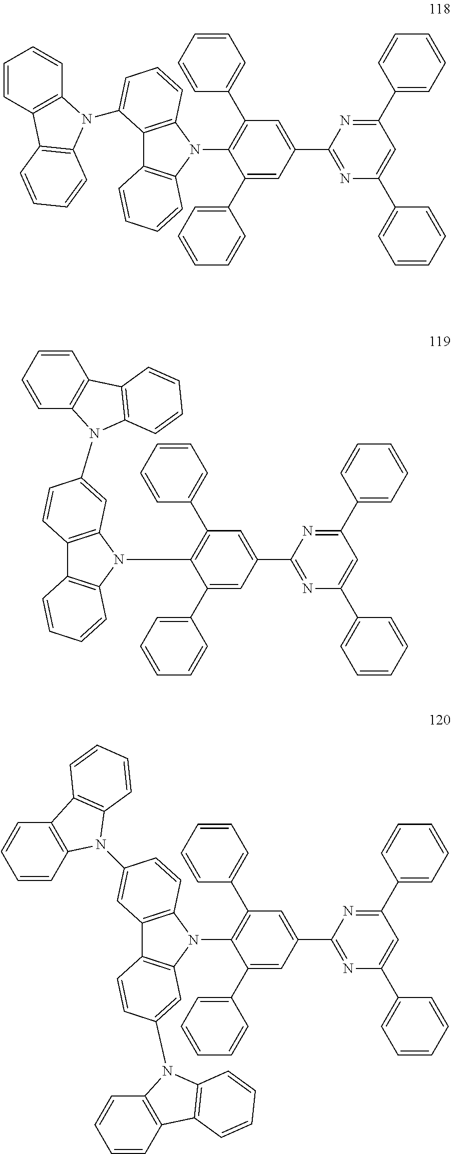 [SMILES: c1ccc(-c2cc(-c3ccccc3)nc(-c3cc(-c4ccccc4)c(-n4c5ccccc5c5c(-n6c7ccccc7c7ccccc76)cccc54)c(-c4ccccc4)c3)n2)cc1 ; c1ccc(-c2cc(-c3ccccc3)nc(-c3cc(-c4ccccc4)c(-n4c5ccccc5c5ccc(-n6c7ccccc7c7ccccc76)cc54)c(-c4ccccc4)c3)n2)cc1 ; c1ccc(-c2cc(-c3ccccc3)nc(-c3cc(-c4ccccc4)c(-n4c5ccc(-n6c7ccccc7c7ccccc76)cc5c5ccc(-n6c7ccccc7c7ccccc76)cc54)c(-c4ccccc4)c3)n2)cc1]

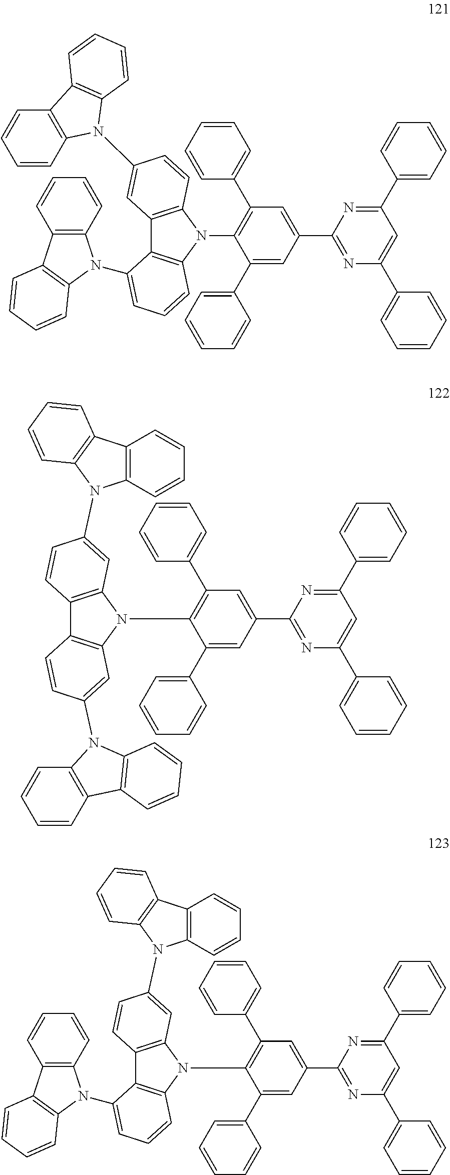 [SMILES: c1ccc(-c2cc(-c3ccccc3)nc(-c3cc(-c4ccccc4)c(-n4c5ccc(-n6c7ccccc7c7ccccc76)cc5c5c(-n6c7ccccc7c7ccccc76)cccc54)c(-c4ccccc4)c3)n2)cc1 ; c1ccc(-c2cc(-c3ccccc3)nc(-c3cc(-c4ccccc4)c(-n4c5cc(-n6c7ccccc7c7ccccc76)ccc5c5ccc(-n6c7ccccc7c7ccccc76)cc54)c(-c4ccccc4)c3)n2)cc1 ; c1ccc(-c2cc(-c3ccccc3)nc(-c3cc(-c4ccccc4)c(-n4c5cc(-n6c7ccccc7c7ccccc76)ccc5c5c(-n6c7ccccc7c7ccccc76)cccc54)c(-c4ccccc4)c3)n2)cc1]

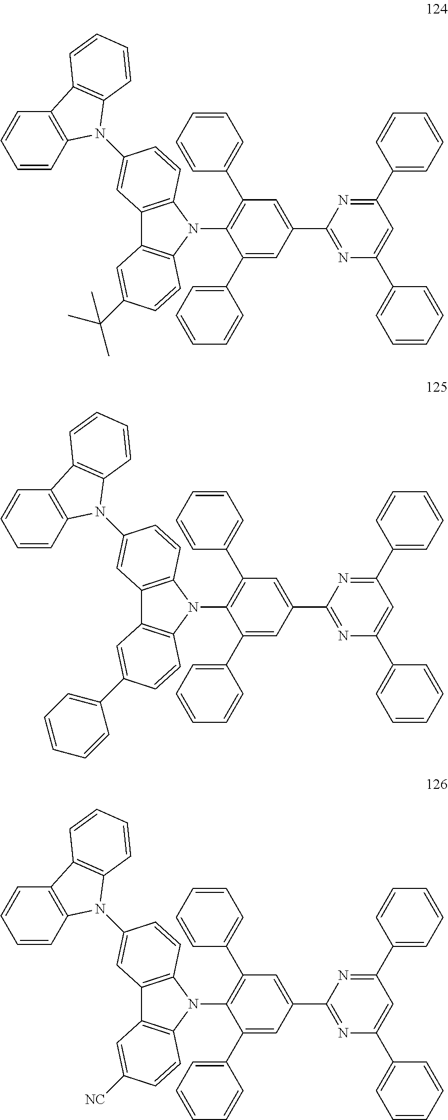 [SMILES: CC(C)(C)c1ccc2c(c1)c1cc(-n3c4ccccc4c4ccccc43)ccc1n2-c1c(-c2ccccc2)cc(-c2nc(-c3ccccc3)cc(-c3ccccc3)n2)cc1-c1ccccc1 ; c1ccc(-c2ccc3c(c2)c2cc(-n4c5ccccc5c5ccccc54)ccc2n3-c2c(-c3ccccc3)cc(-c3nc(-c4ccccc4)cc(-c4ccccc4)n3)cc2-c2ccccc2)cc1 ; N#Cc1ccc2c(c1)c1cc(-n3c4ccccc4c4ccccc43)ccc1n2-c1c(-c2ccccc2)cc(-c2nc(-c3ccccc3)cc(-c3ccccc3)n2)cc1-c1ccccc1]

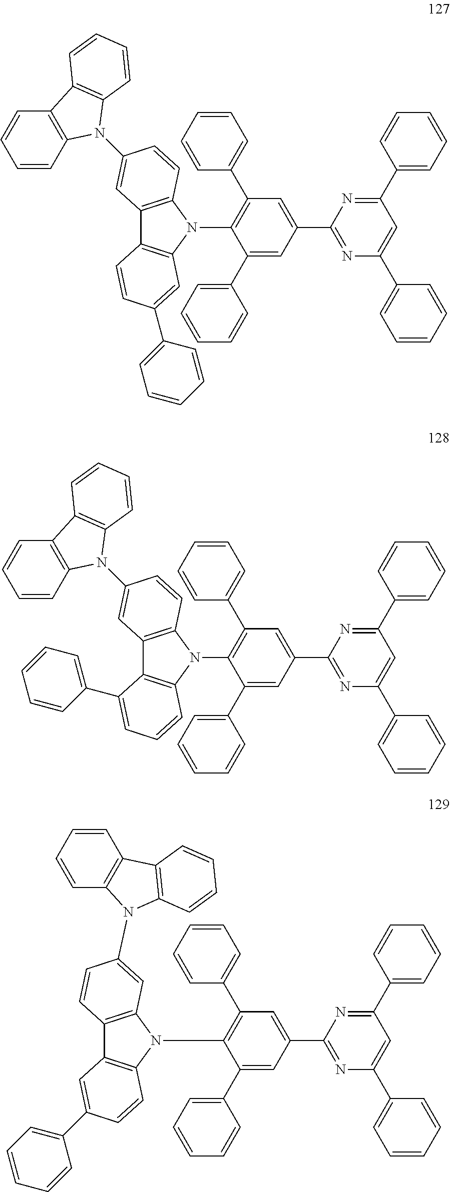 [SMILES: c1ccc(-c2ccc3c4cc(-n5c6ccccc6c6ccccc65)ccc4n(-c4c(-c5ccccc5)cc(-c5nc(-c6ccccc6)cc(-c6ccccc6)n5)cc4-c4ccccc4)c3c2)cc1 ; c1ccc(-c2cc(-c3ccccc3)nc(-c3cc(-c4ccccc4)c(-n4c5ccc(-n6c7ccccc7c7ccccc76)cc5c5c(-c6ccccc6)cccc54)c(-c4ccccc4)c3)n2)cc1 ; c1ccc(-c2ccc3c(c2)c2ccc(-n4c5ccccc5c5ccccc54)cc2n3-c2c(-c3ccccc3)cc(-c3nc(-c4ccccc4)cc(-c4ccccc4)n3)cc2-c2ccccc2)cc1]

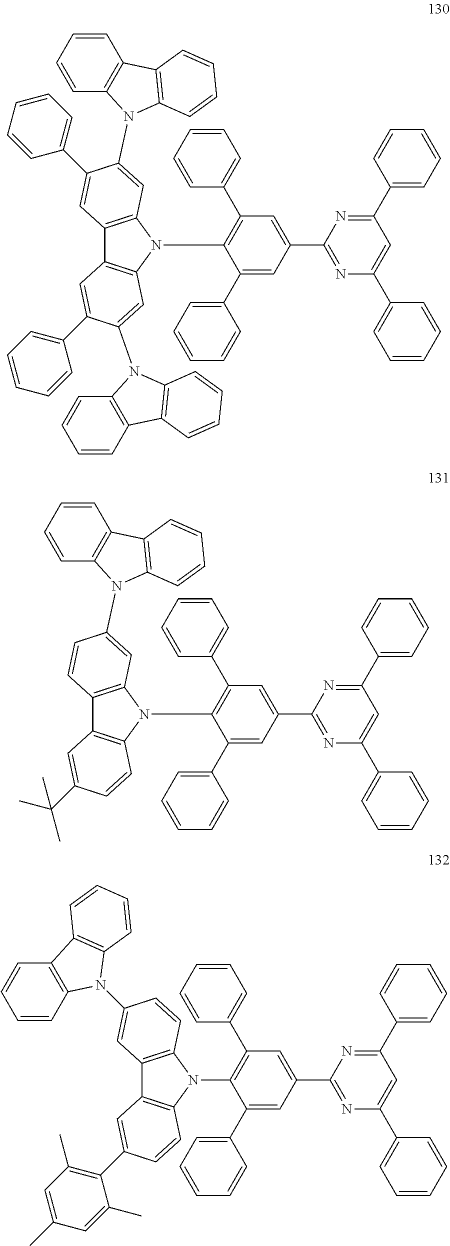 [SMILES: c1ccc(-c2cc(-c3ccccc3)nc(-c3cc(-c4ccccc4)c(-n4c5cc(-n6c7ccccc7c7ccccc76)c(-c6ccccc6)cc5c5cc(-c6ccccc6)c(-n6c7ccccc7c7ccccc76)cc54)c(-c4ccccc4)c3)n2)cc1 ; CC(C)(C)c1ccc2c(c1)c1ccc(-n3c4ccccc4c4ccccc43)cc1n2-c1c(-c2ccccc2)cc(-c2nc(-c3ccccc3)cc(-c3ccccc3)n2)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccccc4)cc(-c4ccccc4)n3)cc2-c2ccccc2)c(C)c1]

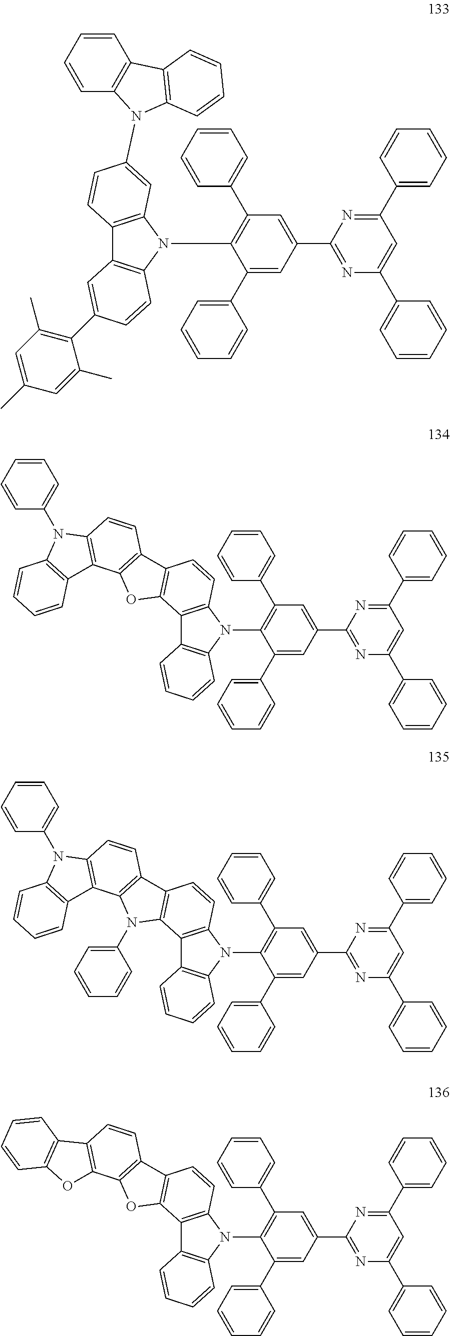 [SMILES: Cc1cc(C)c(-c2ccc3c(c2)c2ccc(-n4c5ccccc5c5ccccc54)cc2n3-c2c(-c3ccccc3)cc(-c3nc(-c4ccccc4)cc(-c4ccccc4)n3)cc2-c2ccccc2)c(C)c1 ; c1ccc(-c2cc(-c3ccccc3)nc(-c3cc(-c4ccccc4)c(-n4c5ccccc5c5c6oc7c(ccc8c7c7ccccc7n8-c7ccccc7)c6ccc54)c(-c4ccccc4)c3)n2)cc1 ; c1ccc(-c2cc(-c3ccccc3)nc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)n2)cc1 ; c1ccc(-c2cc(-c3ccccc3)nc(-c3cc(-c4ccccc4)c(-n4c5ccccc5c5c6oc7c(ccc8c9ccccc9oc87)c6ccc54)c(-c4ccccc4)c3)n2)cc1]

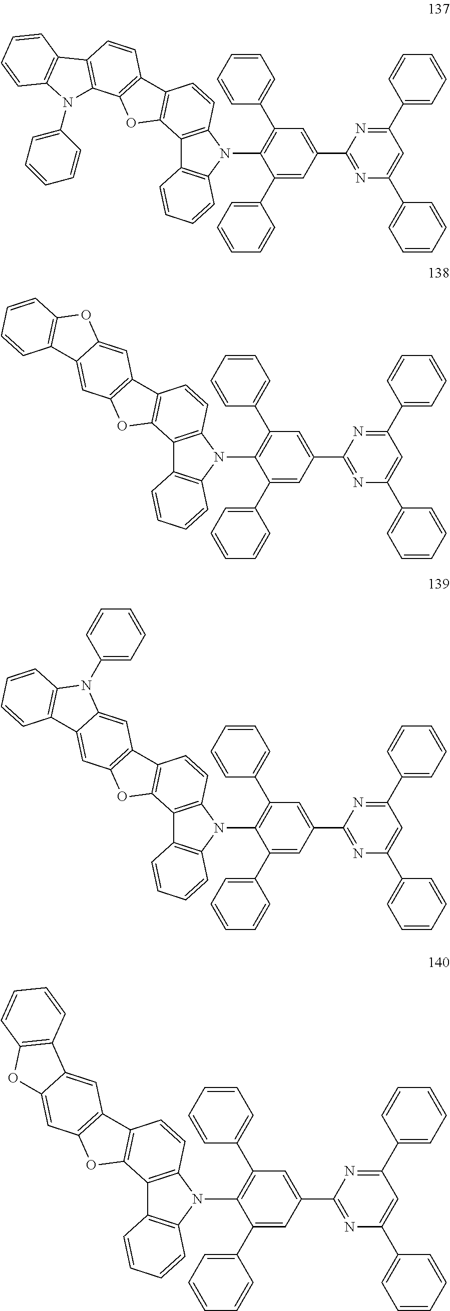 [SMILES: c1ccc(-c2cc(-c3ccccc3)nc(-c3cc(-c4ccccc4)c(-n4c5ccccc5c5c6oc7c(ccc8c9ccccc9n(-c9ccccc9)c87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7cc8c(cc7c6ccc54)oc4ccccc48)c(-c4ccccc4)c3)n2)cc1 ; c1ccc(-c2cc(-c3ccccc3)nc(-c3cc(-c4ccccc4)c(-n4c5ccccc5c5c6oc7cc8c9ccccc9n(-c9ccccc9)c8cc7c6ccc54)c(-c4ccccc4)c3)n2)cc1 ; c1ccc(-c2cc(-c3ccccc3)nc(-c3cc(-c4ccccc4)c(-n4c5ccccc5c5c6oc7cc8oc9ccccc9c8cc7c6ccc54)c(-c4ccccc4)c3)n2)cc1]

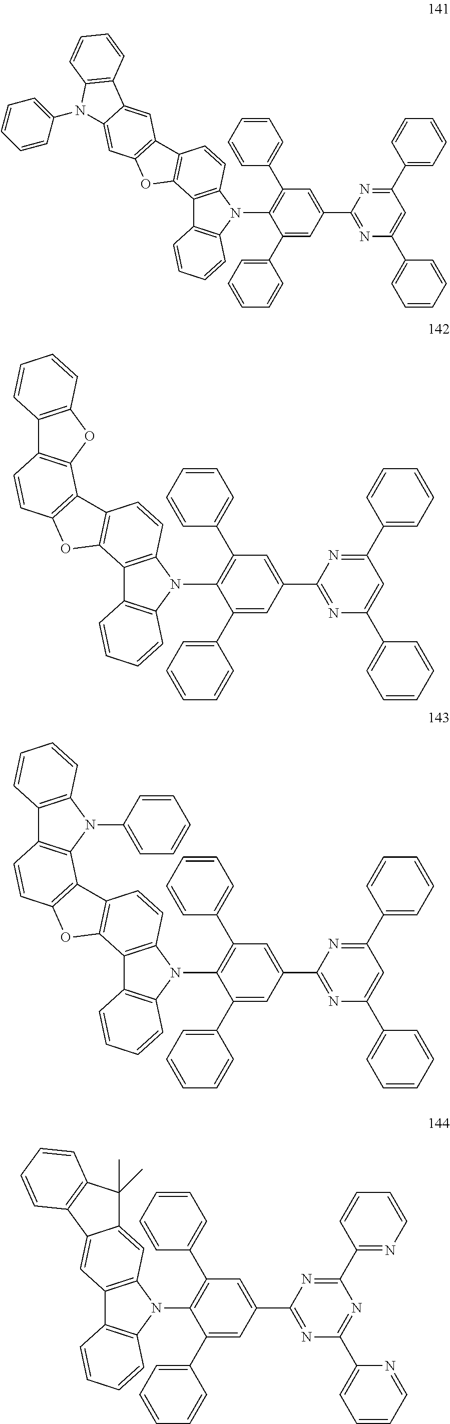 [SMILES: c1ccc(-c2cc(-c3ccccc3)nc(-c3cc(-c4ccccc4)c(-n4c5ccccc5c5c6oc7cc8c(cc7c6ccc54)c4ccccc4n8-c4ccccc4)c(-c4ccccc4)c3)n2)cc1 ; c1ccc(-c2cc(-c3ccccc3)nc(-c3cc(-c4ccccc4)c(-n4c5ccccc5c5c6oc7ccc8c9ccccc9oc8c7c6ccc54)c(-c4ccccc4)c3)n2)cc1 ; c1ccc(-c2cc(-c3ccccc3)nc(-c3cc(-c4ccccc4)c(-n4c5ccccc5c5c6oc7ccc8c9ccccc9n(-c9ccccc9)c8c7c6ccc54)c(-c4ccccc4)c3)n2)cc1 ; CC1(C)c2ccccc2-c2cc3c4ccccc4n(-c4c(-c5ccccc5)cc(-c5nc(-c6ccccn6)nc(-c6ccccn6)n5)cc4-c4ccccc4)c3cc21]

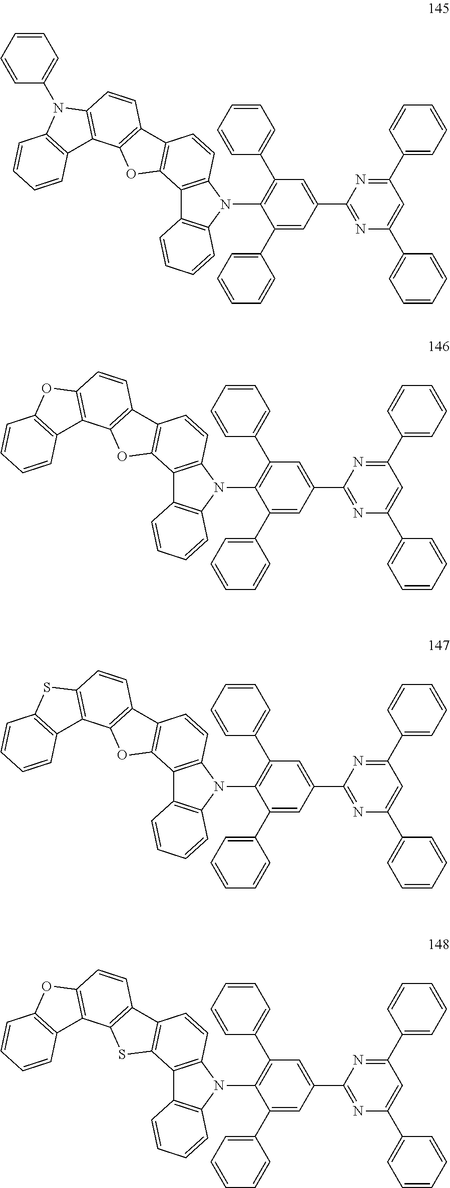 [SMILES: c1ccc(-c2cc(-c3ccccc3)nc(-c3cc(-c4ccccc4)c(-n4c5ccccc5c5c6oc7c(ccc8c7c7ccccc7n8-c7ccccc7)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(ccc8oc9ccccc9c87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(ccc8sc9ccccc9c87)c6ccc54)c(-c4ccccc4)c3)n2)cc1 ; c1ccc(-c2cc(-c3ccccc3)nc(-c3cc(-c4ccccc4)c(-n4c5ccccc5c5c6sc7c(ccc8oc9ccccc9c87)c6ccc54)c(-c4ccccc4)c3)n2)cc1]

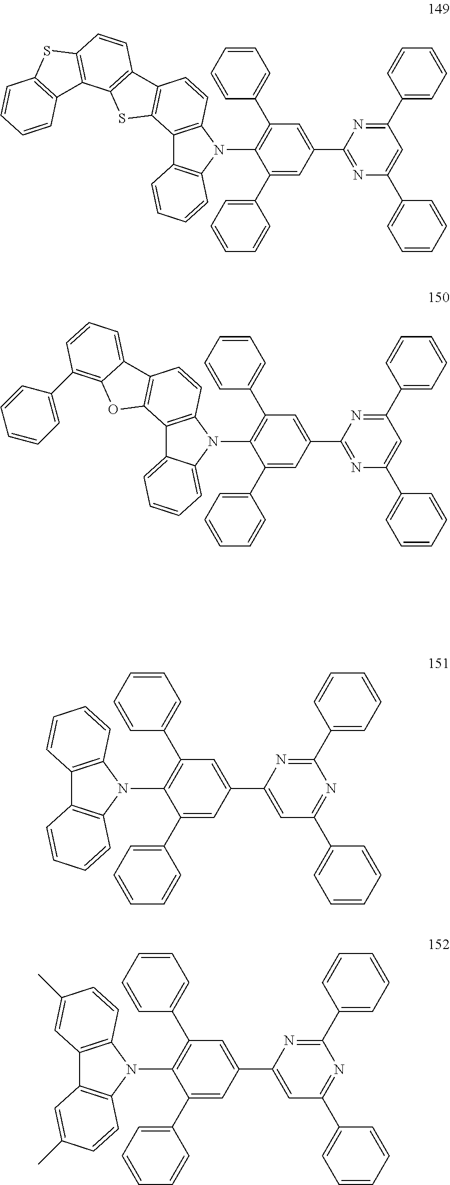 [SMILES: c1ccc(-c2cc(-c3ccccc3)nc(-c3cc(-c4ccccc4)c(-n4c5ccccc5c5c6sc7c(ccc8sc9ccccc9c87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(-c8ccccc8)cccc7c6ccc54)c(-c4ccccc4)c3)n2)cc1 ; c1ccc(-c2cc(-c3cc(-c4ccccc4)c(-n4c5ccccc5c5ccccc54)c(-c4ccccc4)c3)nc(-c3ccccc3)n2)cc1 ; Cc1ccc2c(c1)c1cc(C)ccc1n2-c1c(-c2ccccc2)cc(-c2cc(-c3ccccc3)nc(-c3ccccc3)n2)cc1-c1ccccc1]

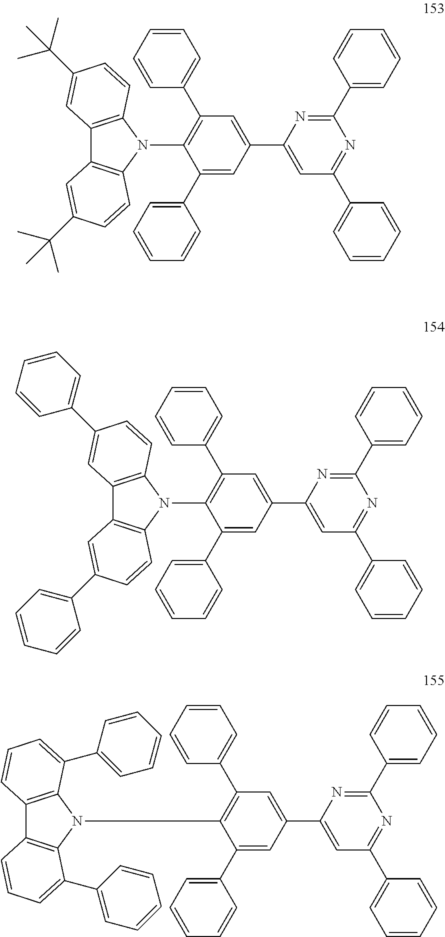 [SMILES: CC(C)(C)c1ccc2c(c1)c1cc(C(C)(C)C)ccc1n2-c1c(-c2ccccc2)cc(-c2cc(-c3ccccc3)nc(-c3ccccc3)n2)cc1-c1ccccc1 ; c1ccc(-c2ccc3c(c2)c2cc(-c4ccccc4)ccc2n3-c2c(-c3ccccc3)cc(-c3cc(-c4ccccc4)nc(-c4ccccc4)n3)cc2-c2ccccc2)cc1 ; c1ccc(-c2cc(-c3cc(-c4ccccc4)c(-n4c5c(-c6ccccc6)cccc5c5cccc(-c6ccccc6)c54)c(-c4ccccc4)c3)nc(-c3ccccc3)n2)cc1]

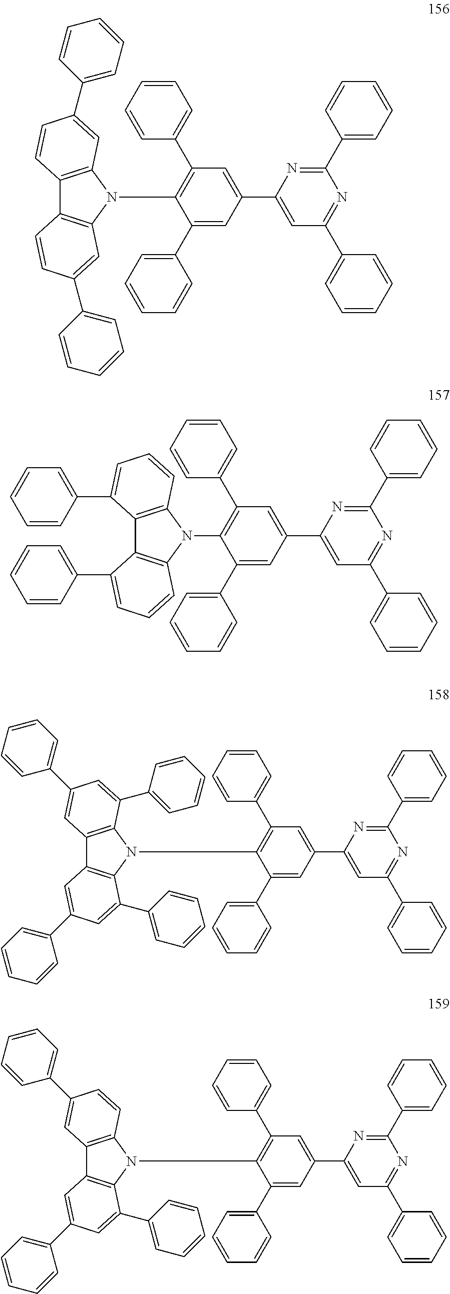 [SMILES: c1ccc(-c2ccc3c4ccc(-c5ccccc5)cc4n(-c4c(-c5ccccc5)cc(-c5cc(-c6ccccc6)nc(-c6ccccc6)n5)cc4-c4ccccc4)c3c2)cc1 ; c1ccc(-c2cc(-c3cc(-c4ccccc4)c(-n4c5cccc(-c6ccccc6)c5c5c(-c6ccccc6)cccc54)c(-c4ccccc4)c3)nc(-c3ccccc3)n2)cc1 ; c1ccc(-c2cc(-c3ccccc3)c3c(c2)c2cc(-c4ccccc4)cc(-c4ccccc4)c2n3-c2c(-c3ccccc3)cc(-c3cc(-c4ccccc4)nc(-c4ccccc4)n3)cc2-c2ccccc2)cc1 ; c1ccc(-c2ccc3c(c2)c2cc(-c4ccccc4)cc(-c4ccccc4)c2n3-c2c(-c3ccccc3)cc(-c3cc(-c4ccccc4)nc(-c4ccccc4)n3)cc2-c2ccccc2)cc1]

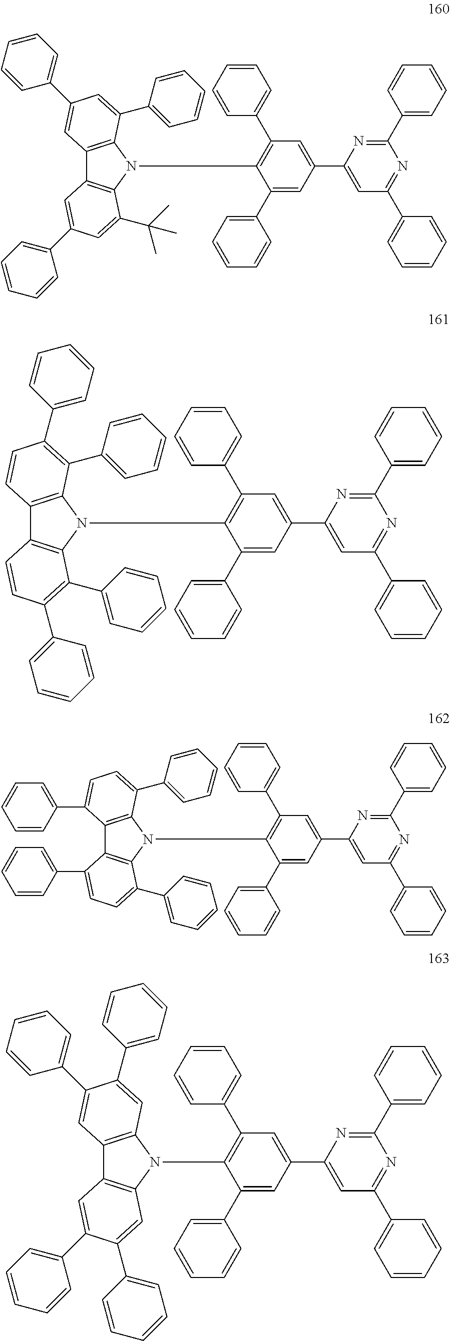

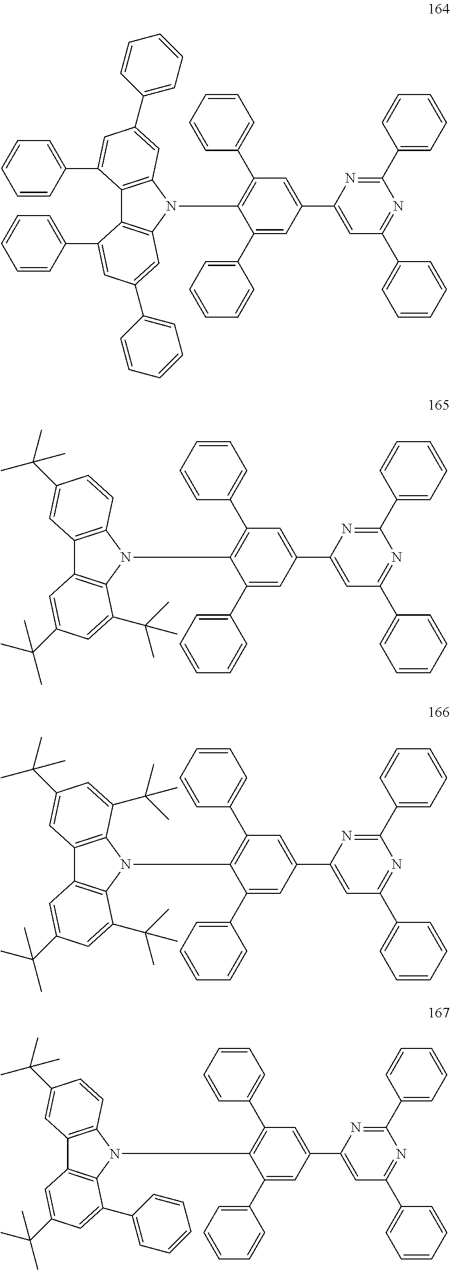 [SMILES: c1ccc(-c2cc(-c3ccccc3)c3c4c(-c5ccccc5)cc(-c5ccccc5)cc4n(-c4c(-c5ccccc5)cc(-c5cc(-c6ccccc6)nc(-c6ccccc6)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2cc(-c3ccccc3)nc(-c3ccccc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2cc(-c3ccccc3)nc(-c3ccccc3)n2)cc1-c1ccccc1 ; CC(C)(C)c1ccc2c(c1)c1cc(C(C)(C)C)cc(-c3ccccc3)c1n2-c1c(-c2ccccc2)cc(-c2cc(-c3ccccc3)nc(-c3ccccc3)n2)cc1-c1ccccc1]

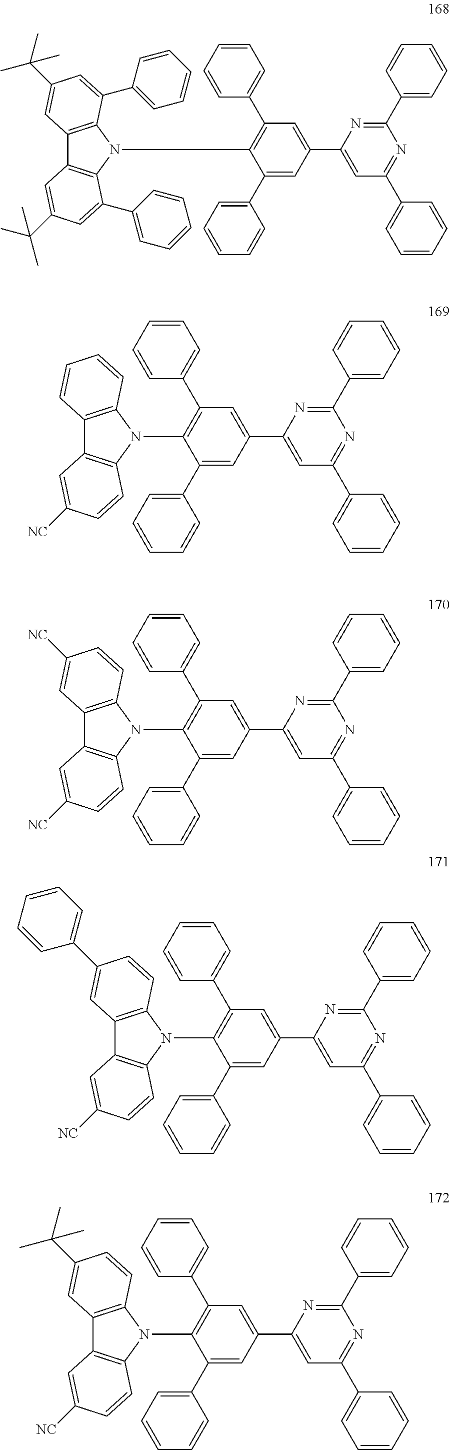

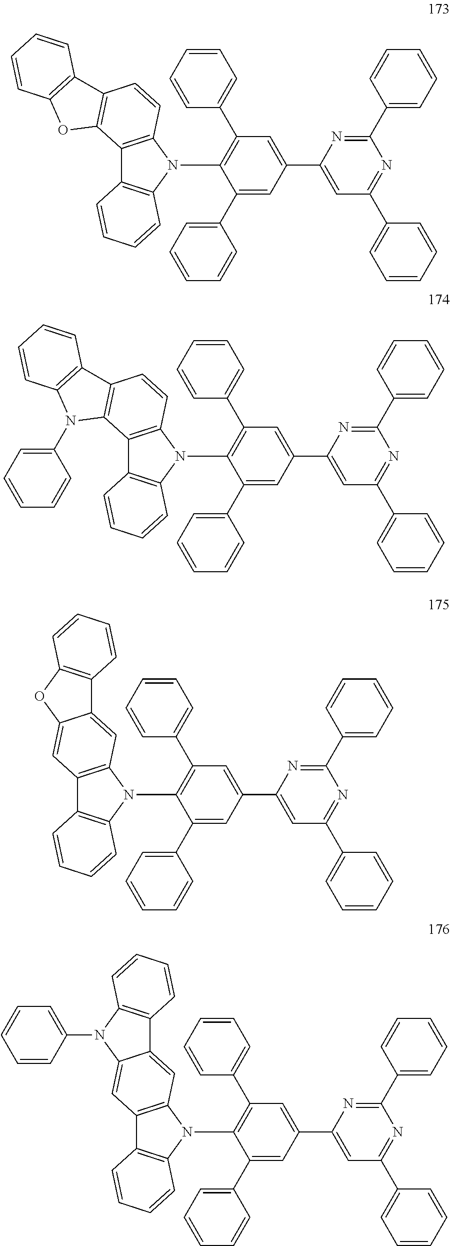

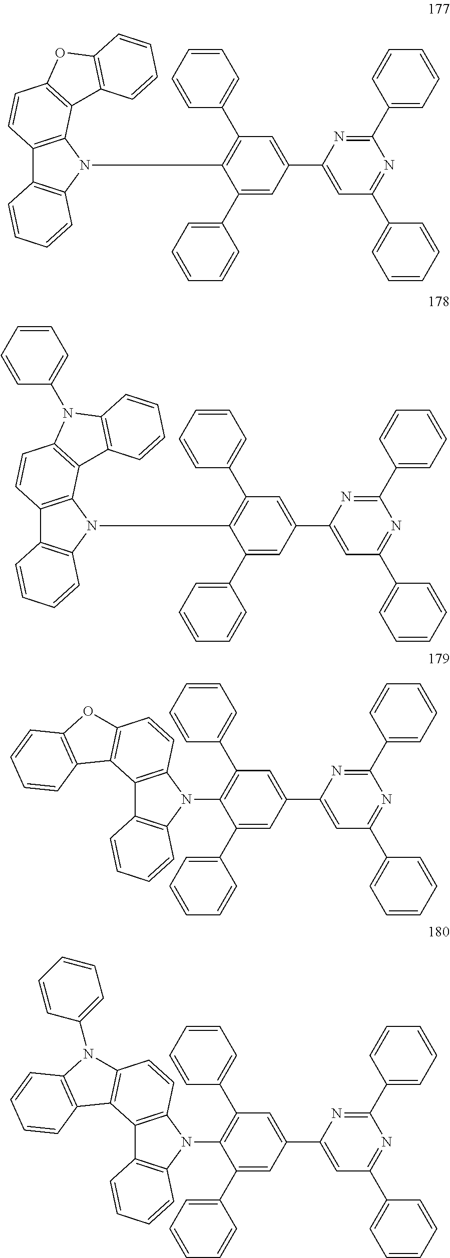

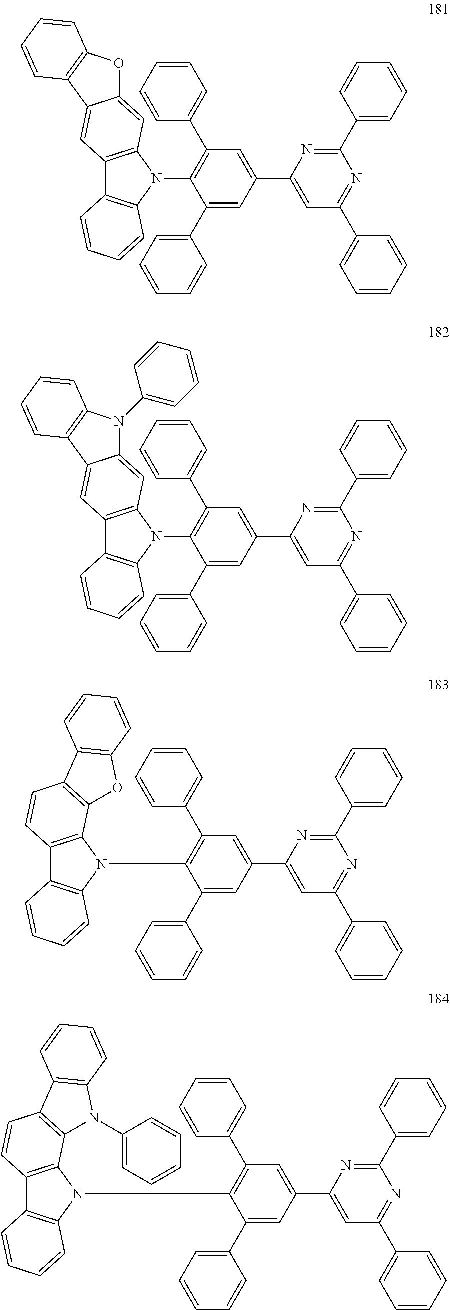 [SMILES: c1ccc(-c2cc(-c3cc(-c4ccccc4)c(-n4c5ccccc5c5cc6c(cc54)oc4ccccc46)c(-c4ccccc4)c3)nc(-c3ccccc3)n2)cc1 ; c1ccc(-c2cc(-c3cc(-c4ccccc4)c(-n4c5ccccc5c5cc6c7ccccc7n(-c7ccccc7)c6cc54)c(-c4ccccc4)c3)nc(-c3ccccc3)n2)cc1 ; c1ccc(-c2cc(-c3cc(-c4ccccc4)c(-n4c5ccccc5c5ccc6c7ccccc7oc6c54)c(-c4ccccc4)c3)nc(-c3ccccc3)n2)cc1 ; c1ccc(-c2cc(-c3cc(-c4ccccc4)c(-n4c5ccccc5c5ccc6c7ccccc7n(-c7ccccc7)c6c54)c(-c4ccccc4)c3)nc(-c3ccccc3)n2)cc1]

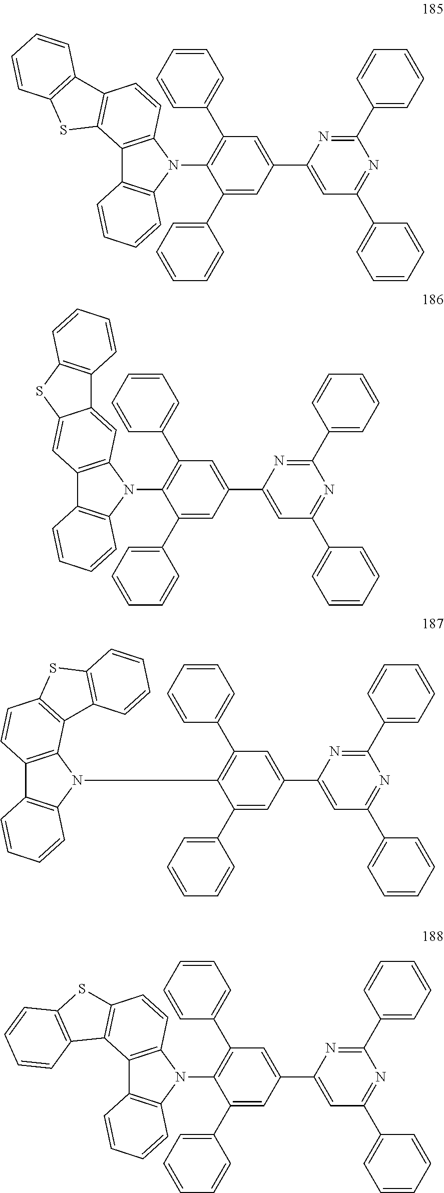 [SMILES: c1ccc(-c2cc(-c3cc(-c4ccccc4)c(-n4c5ccccc5c5c6sc7ccccc7c6ccc54)c(-c4ccccc4)c3)nc(-c3ccccc3)n2)cc1 ; c1ccc(-c2cc(-c3cc(-c4ccccc4)c(-n4c5ccccc5c5cc6sc7ccccc7c6cc54)c(-c4ccccc4)c3)nc(-c3ccccc3)n2)cc1 ; c1ccc(-c2cc(-c3cc(-c4ccccc4)c(-n4c5ccccc5c5ccc6sc7ccccc7c6c54)c(-c4ccccc4)c3)nc(-c3ccccc3)n2)cc1 ; c1ccc(-c2cc(-c3cc(-c4ccccc4)c(-n4c5ccccc5c5c6c(ccc54)sc4ccccc46)c(-c4ccccc4)c3)nc(-c3ccccc3)n2)cc1]

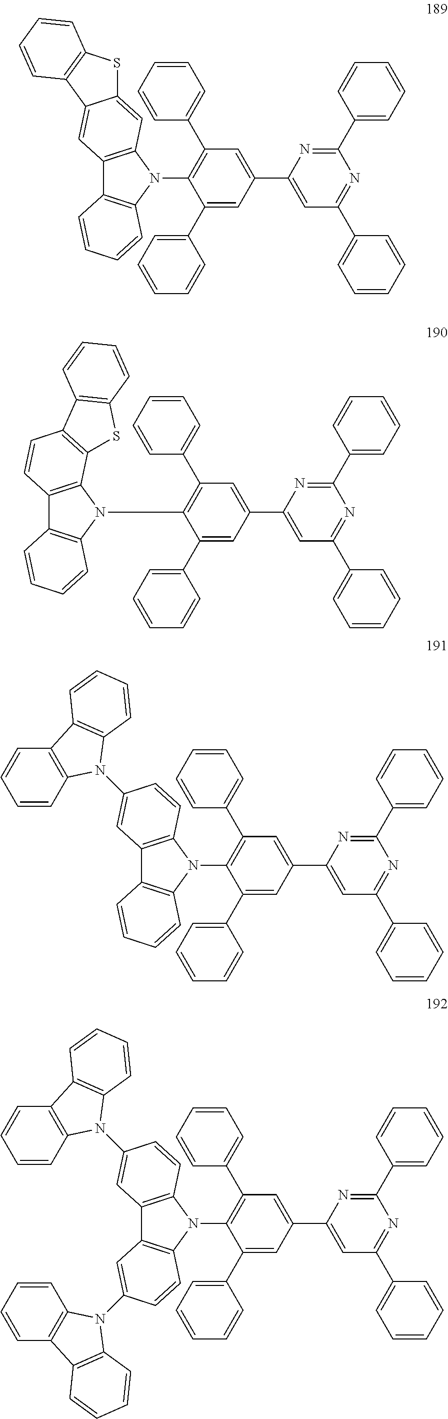 [SMILES: c1ccc(-c2cc(-c3cc(-c4ccccc4)c(-n4c5ccccc5c5cc6c(cc54)sc4ccccc46)c(-c4ccccc4)c3)nc(-c3ccccc3)n2)cc1 ; c1ccc(-c2cc(-c3cc(-c4ccccc4)c(-n4c5ccccc5c5ccc6c7ccccc7sc6c54)c(-c4ccccc4)c3)nc(-c3ccccc3)n2)cc1 ; c1ccc(-c2cc(-c3cc(-c4ccccc4)c(-n4c5ccccc5c5cc(-n6c7ccccc7c7ccccc76)ccc54)c(-c4ccccc4)c3)nc(-c3ccccc3)n2)cc1 ; c1ccc(-c2cc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)nc(-c3ccccc3)n2)cc1]

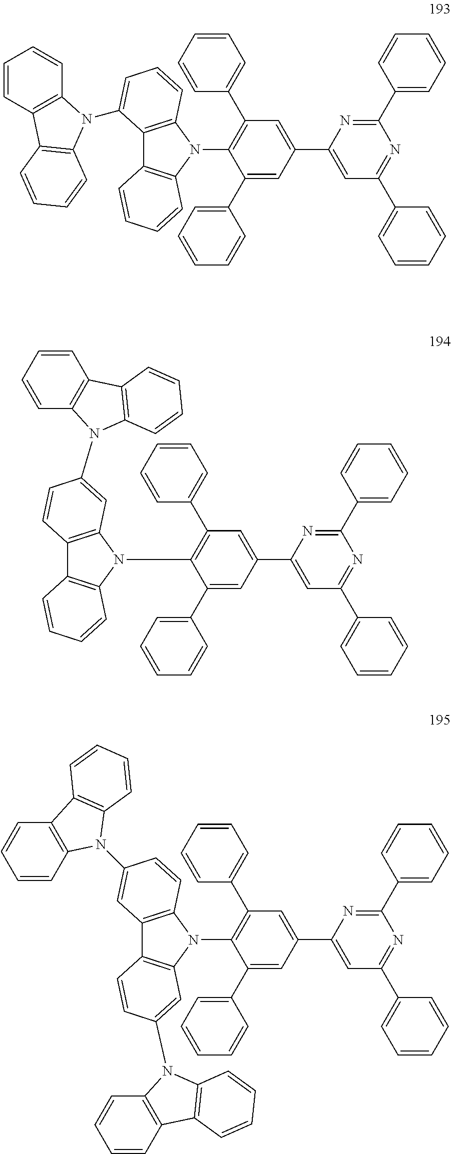 [SMILES: c1ccc(-c2cc(-c3cc(-c4ccccc4)c(-n4c5ccccc5c5c(-n6c7ccccc7c7ccccc76)cccc54)c(-c4ccccc4)c3)nc(-c3ccccc3)n2)cc1 ; c1ccc(-c2cc(-c3cc(-c4ccccc4)c(-n4c5ccccc5c5ccc(-n6c7ccccc7c7ccccc76)cc54)c(-c4ccccc4)c3)nc(-c3ccccc3)n2)cc1 ; c1ccc(-c2cc(-c3cc(-c4ccccc4)c(-n4c5ccc(-n6c7ccccc7c7ccccc76)cc5c5ccc(-n6c7ccccc7c7ccccc76)cc54)c(-c4ccccc4)c3)nc(-c3ccccc3)n2)cc1]

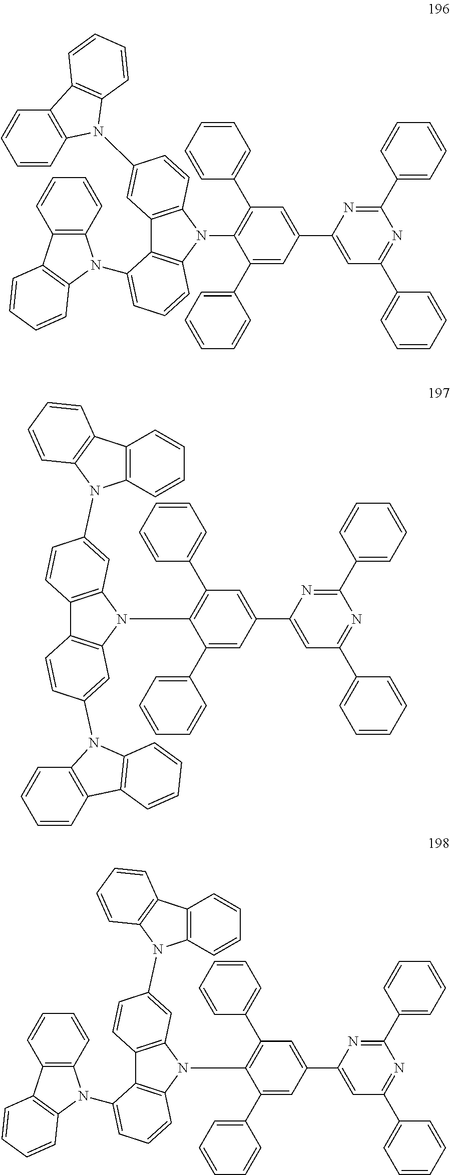 [SMILES: c1ccc(-c2cc(-c3cc(-c4ccccc4)c(-n4c5ccc(-n6c7ccccc7c7ccccc76)cc5c5c(-n6c7ccccc7c7ccccc76)cccc54)c(-c4ccccc4)c3)nc(-c3ccccc3)n2)cc1 ; c1ccc(-c2cc(-c3cc(-c4ccccc4)c(-n4c5cc(-n6c7ccccc7c7ccccc76)ccc5c5ccc(-n6c7ccccc7c7ccccc76)cc54)c(-c4ccccc4)c3)nc(-c3ccccc3)n2)cc1 ; c1ccc(-c2cc(-c3cc(-c4ccccc4)c(-n4c5cc(-n6c7ccccc7c7ccccc76)ccc5c5c(-n6c7ccccc7c7ccccc76)cccc54)c(-c4ccccc4)c3)nc(-c3ccccc3)n2)cc1]

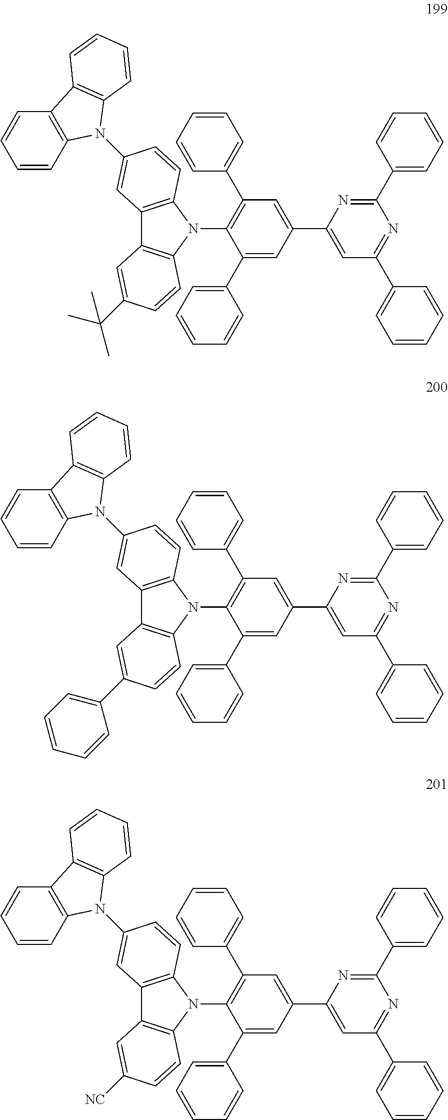 [SMILES: CC(C)(C)c1ccc2c(c1)c1cc(-n3c4ccccc4c4ccccc43)ccc1n2-c1c(-c2ccccc2)cc(-c2cc(-c3ccccc3)nc(-c3ccccc3)n2)cc1-c1ccccc1 ; c1ccc(-c2ccc3c(c2)c2cc(-n4c5ccccc5c5ccccc54)ccc2n3-c2c(-c3ccccc3)cc(-c3cc(-c4ccccc4)nc(-c4ccccc4)n3)cc2-c2ccccc2)cc1 ; N#Cc1ccc2c(c1)c1cc(-n3c4ccccc4c4ccccc43)ccc1n2-c1c(-c2ccccc2)cc(-c2cc(-c3ccccc3)nc(-c3ccccc3)n2)cc1-c1ccccc1]

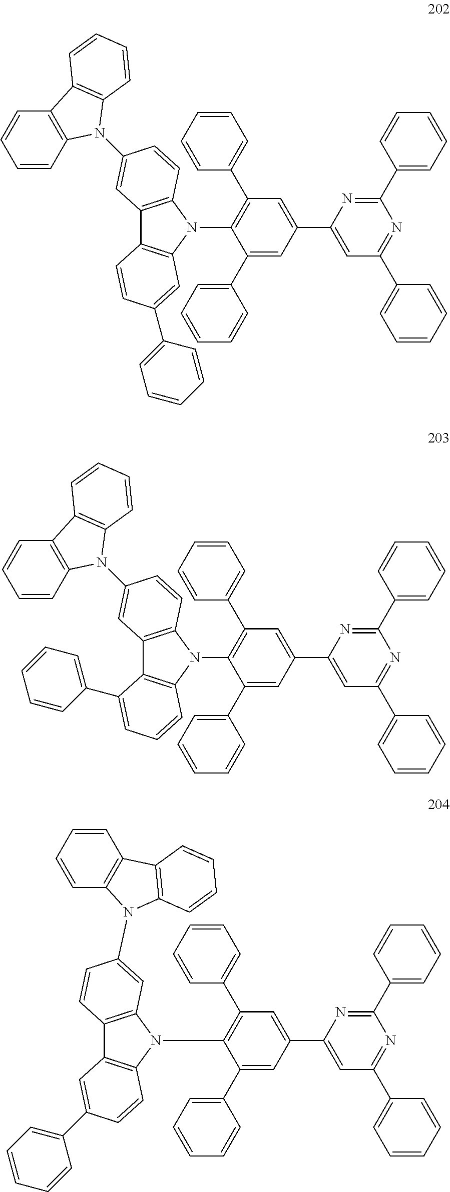 [SMILES: c1ccc(-c2ccc3c4cc(-n5c6ccccc6c6ccccc65)ccc4n(-c4c(-c5ccccc5)cc(-c5cc(-c6ccccc6)nc(-c6ccccc6)n5)cc4-c4ccccc4)c3c2)cc1 ; c1ccc(-c2cc(-c3cc(-c4ccccc4)c(-n4c5ccc(-n6c7ccccc7c7ccccc76)cc5c5c(-c6ccccc6)cccc54)c(-c4ccccc4)c3)nc(-c3ccccc3)n2)cc1 ; c1ccc(-c2ccc3c(c2)c2ccc(-n4c5ccccc5c5ccccc54)cc2n3-c2c(-c3ccccc3)cc(-c3cc(-c4ccccc4)nc(-c4ccccc4)n3)cc2-c2ccccc2)cc1]

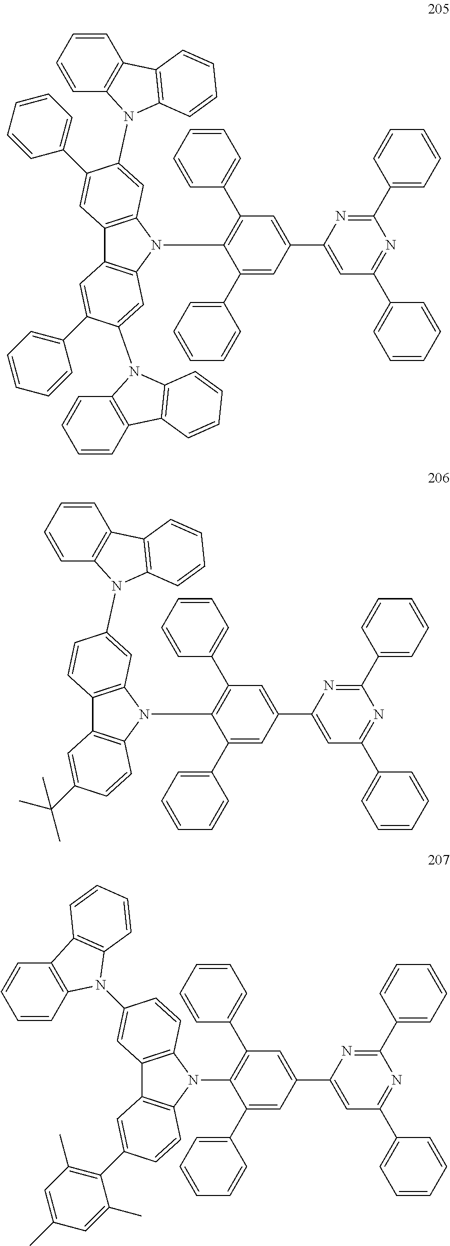 [SMILES: c1ccc(-c2cc(-c3cc(-c4ccccc4)c(-n4c5cc(-n6c7ccccc7c7ccccc76)c(-c6ccccc6)cc5c5cc(-c6ccccc6)c(-n6c7ccccc7c7ccccc76)cc54)c(-c4ccccc4)c3)nc(-c3ccccc3)n2)cc1 ; CC(C)(C)c1ccc2c(c1)c1ccc(-n3c4ccccc4c4ccccc43)cc1n2-c1c(-c2ccccc2)cc(-c2cc(-c3ccccc3)nc(-c3ccccc3)n2)cc1-c1ccccc1 ; Cc1cc(C)c(-c2ccc3c(c2)c2cc(-n4c5ccccc5c5ccccc54)ccc2n3-c2c(-c3ccccc3)cc(-c3cc(-c4ccccc4)nc(-c4ccccc4)n3)cc2-c2ccccc2)c(C)c1]

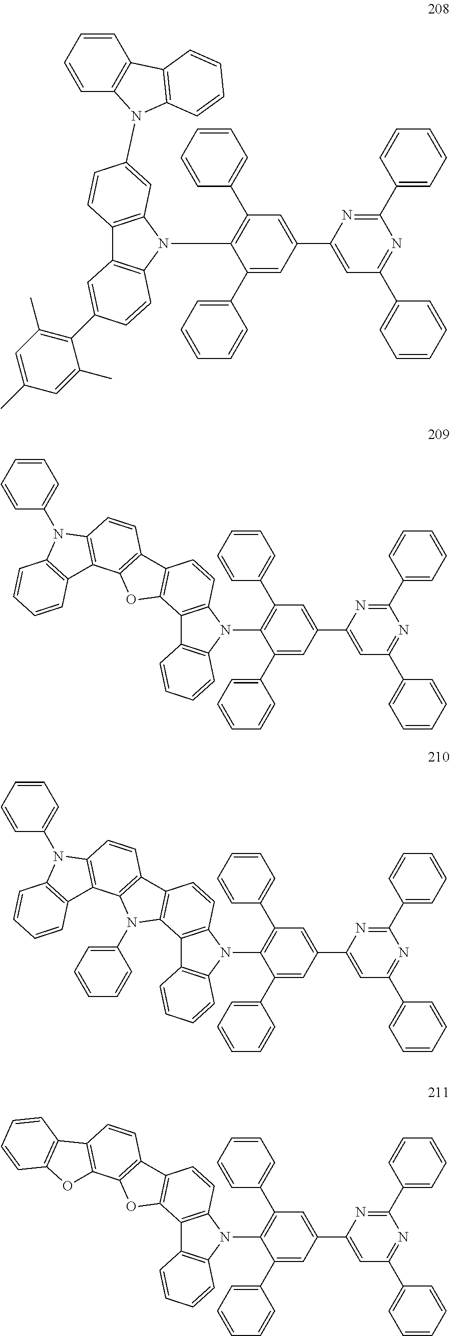 [SMILES: Cc1cc(C)c(-c2ccc3c(c2)c2ccc(-n4c5ccccc5c5ccccc54)cc2n3-c2c(-c3ccccc3)cc(-c3cc(-c4ccccc4)nc(-c4ccccc4)n3)cc2-c2ccccc2)c(C)c1 ; c1ccc(-c2cc(-c3cc(-c4ccccc4)c(-n4c5ccccc5c5c6oc7c(ccc8c7c7ccccc7n8-c7ccccc7)c6ccc54)c(-c4ccccc4)c3)nc(-c3ccccc3)n2)cc1 ; c1ccc(-c2cc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)nc(-c3ccccc3)n2)cc1 ; c1ccc(-c2cc(-c3cc(-c4ccccc4)c(-n4c5ccccc5c5c6oc7c(ccc8c9ccccc9oc87)c6ccc54)c(-c4ccccc4)c3)nc(-c3ccccc3)n2)cc1]

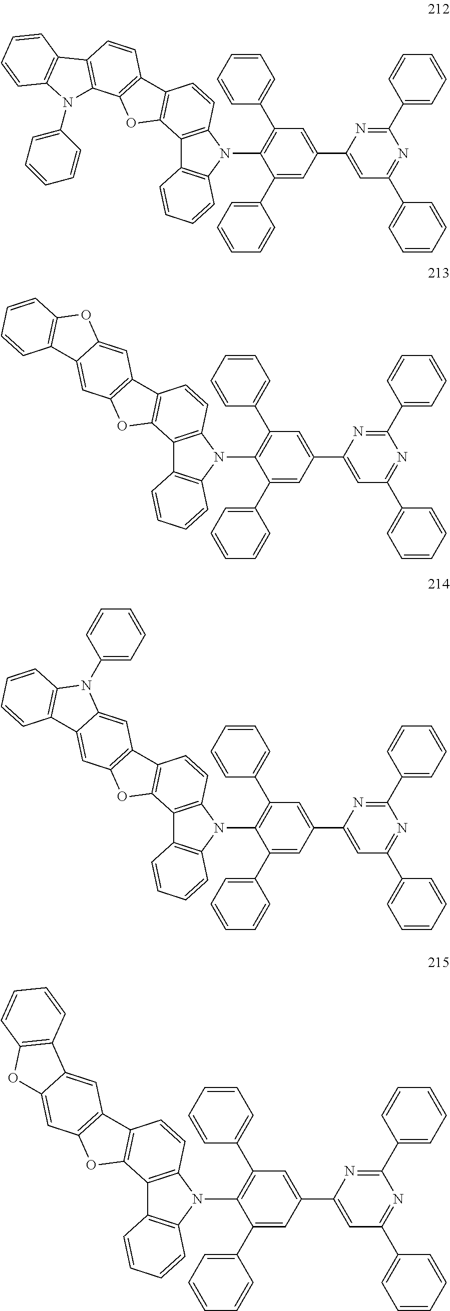 [SMILES: c1ccc(-c2cc(-c3cc(-c4ccccc4)c(-n4c5ccccc5c5c6oc7c(ccc8c9ccccc9n(-c9ccccc9)c87)c6ccc54)c(-c4ccccc4)c3)nc(-c3ccccc3)n2)cc1 ; c1ccc(-c2cc(-c3cc(-c4ccccc4)c(-n4c5ccccc5c5c6oc7cc8c(cc7c6ccc54)oc4ccccc48)c(-c4ccccc4)c3)nc(-c3ccccc3)n2)cc1 ; c1ccc(-c2cc(-c3cc(-c4ccccc4)c(-n4c5ccccc5c5c6oc7cc8c9ccccc9n(-c9ccccc9)c8cc7c6ccc54)c(-c4ccccc4)c3)nc(-c3ccccc3)n2)cc1 ; c1ccc(-c2cc(-c3cc(-c4ccccc4)c(-n4c5ccccc5c5c6oc7cc8oc9ccccc9c8cc7c6ccc54)c(-c4ccccc4)c3)nc(-c3ccccc3)n2)cc1]

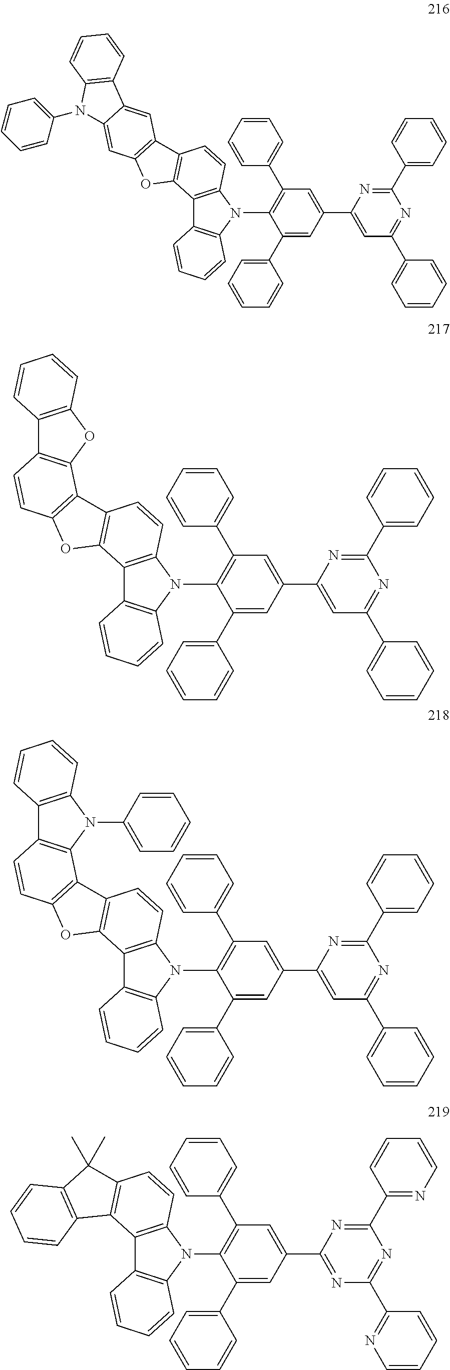 [SMILES: c1ccc(-c2cc(-c3cc(-c4ccccc4)c(-n4c5ccccc5c5c6oc7cc8c(cc7c6ccc54)c4ccccc4n8-c4ccccc4)c(-c4ccccc4)c3)nc(-c3ccccc3)n2)cc1 ; c1ccc(-c2cc(-c3cc(-c4ccccc4)c(-n4c5ccccc5c5c6oc7ccc8c9ccccc9oc8c7c6ccc54)c(-c4ccccc4)c3)nc(-c3ccccc3)n2)cc1 ; c1ccc(-c2cc(-c3cc(-c4ccccc4)c(-n4c5ccccc5c5c6oc7ccc8c9ccccc9n(-c9ccccc9)c8c7c6ccc54)c(-c4ccccc4)c3)nc(-c3ccccc3)n2)cc1 ; CC1(C)c2ccccc2-c2c1ccc1c2c2ccccc2n1-c1c(-c2ccccc2)cc(-c2nc(-c3ccccn3)nc(-c3ccccn3)n2)cc1-c1ccccc1]

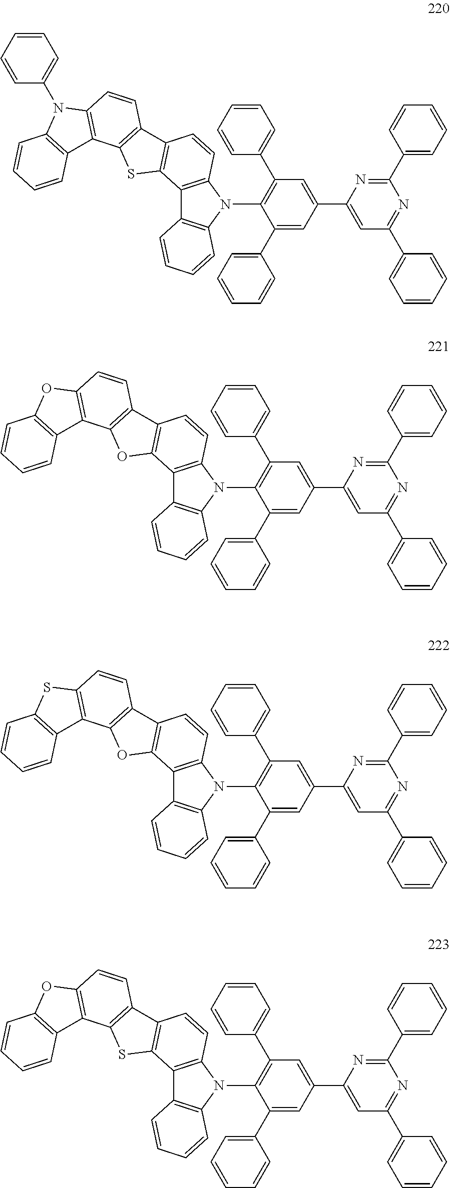 [SMILES: c1ccc(-c2cc(-c3cc(-c4ccccc4)c(-n4c5ccccc5c5c6sc7c(ccc8c7c7ccccc7n8-c7ccccc7)c6ccc54)c(-c4ccccc4)c3)nc(-c3ccccc3)n2)cc1 ; c1ccc(-c2cc(-c3cc(-c4ccccc4)c(-n4c5ccccc5c5c6oc7c(ccc8oc9ccccc9c87)c6ccc54)c(-c4ccccc4)c3)nc(-c3ccccc3)n2)cc1 ; c1ccc(-c2cc(-c3cc(-c4ccccc4)c(-n4c5ccccc5c5c6oc7c(ccc8sc9ccccc9c87)c6ccc54)c(-c4ccccc4)c3)nc(-c3ccccc3)n2)cc1 ; c1ccc(-c2cc(-c3cc(-c4ccccc4)c(-n4c5ccccc5c5c6sc7c(ccc8oc9ccccc9c87)c6ccc54)c(-c4ccccc4)c3)nc(-c3ccccc3)n2)cc1]

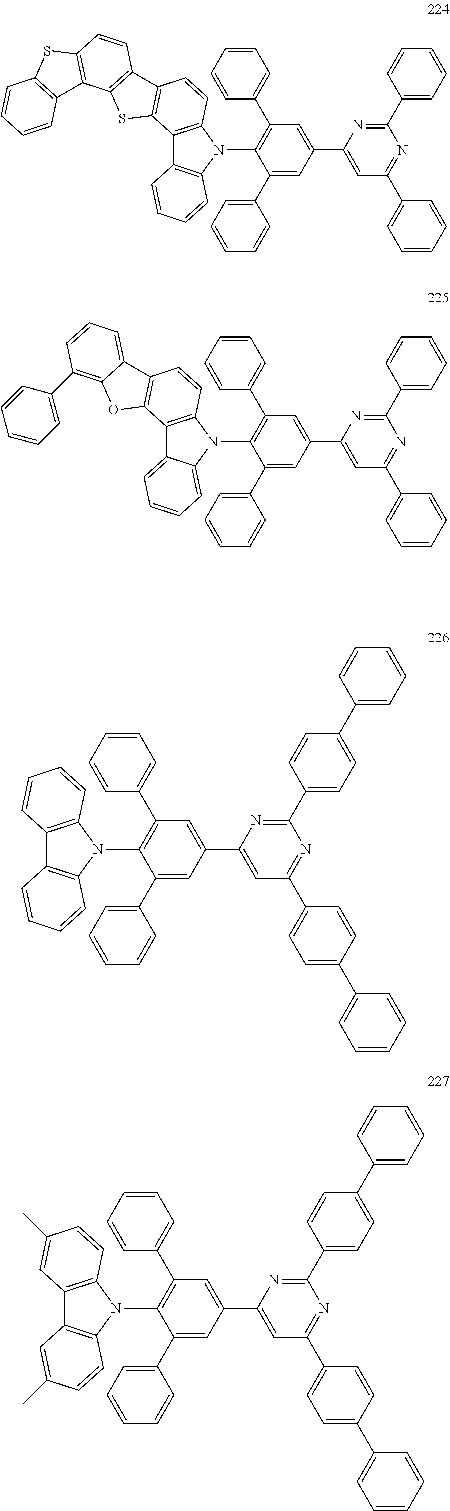 [SMILES: c1ccc(-c2cc(-c3cc(-c4ccccc4)c(-n4c5ccccc5c5c6sc7c(ccc8sc9ccccc9c87)c6ccc54)c(-c4ccccc4)c3)nc(-c3ccccc3)n2)cc1 ; c1ccc(-c2cc(-c3cc(-c4ccccc4)c(-n4c5ccccc5c5c6oc7c(-c8ccccc8)cccc7c6ccc54)c(-c4ccccc4)c3)nc(-c3ccccc3)n2)cc1 ; c1ccc(-c2ccc(-c3cc(-c4cc(-c5ccccc5)c(-n5c6ccccc6c6ccccc65)c(-c5ccccc5)c4)nc(-c4ccc(-c5ccccc5)cc4)n3)cc2)cc1 ; Cc1ccc2c(c1)c1cc(C)ccc1n2-c1c(-c2ccccc2)cc(-c2cc(-c3ccc(-c4ccccc4)cc3)nc(-c3ccc(-c4ccccc4)cc3)n2)cc1-c1ccccc1]

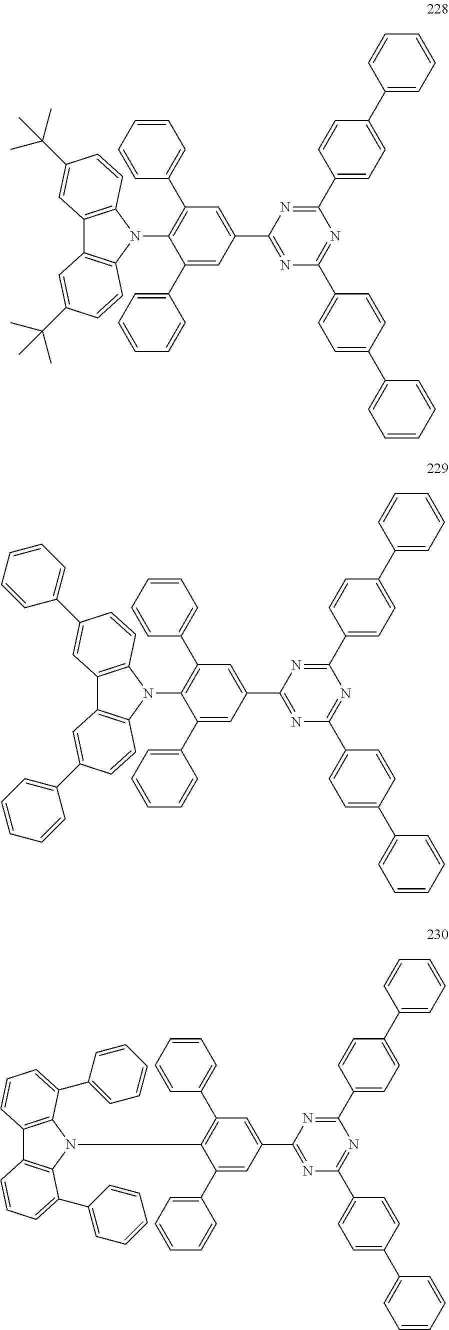 [SMILES: CC(C)(C)c1ccc2c(c1)c1cc(C(C)(C)C)ccc1n2-c1c(-c2ccccc2)cc(-c2nc(-c3ccc(-c4ccccc4)cc3)nc(-c3ccc(-c4ccccc4)cc3)n2)cc1-c1ccccc1 ; c1ccc(-c2ccc(-c3nc(-c4ccc(-c5ccccc5)cc4)nc(-c4cc(-c5ccccc5)c(-n5c6ccc(-c7ccccc7)cc6c6cc(-c7ccccc7)ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6c(-c7ccccc7)cccc6c6cccc(-c7ccccc7)c65)c(-c5ccccc5)c4)n3)cc2)cc1]

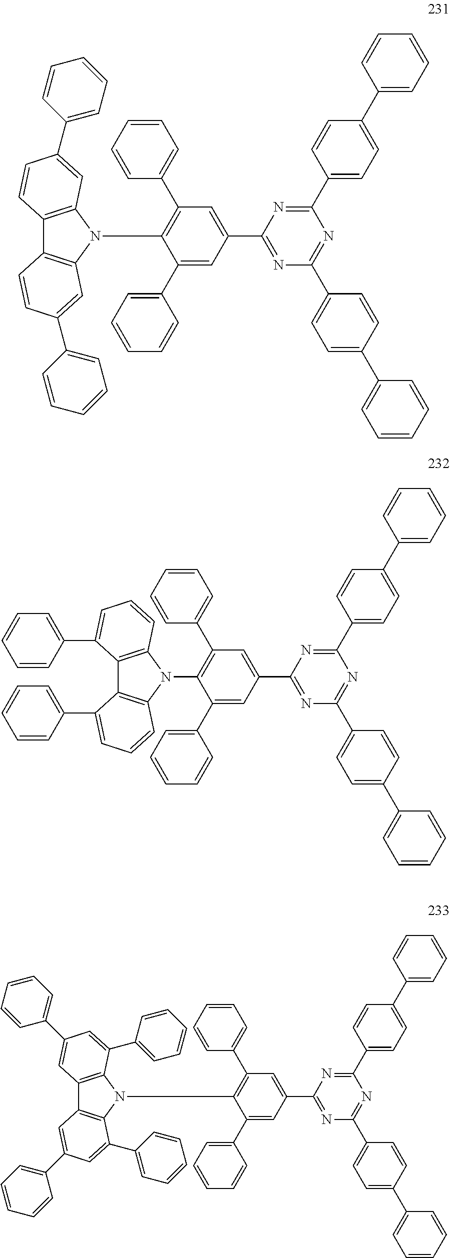 [SMILES: c1ccc(-c2ccc(-c3nc(-c4ccc(-c5ccccc5)cc4)nc(-c4cc(-c5ccccc5)c(-n5c6cc(-c7ccccc7)ccc6c6ccc(-c7ccccc7)cc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6cccc(-c7ccccc7)c6c6c(-c7ccccc7)cccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6c(-c7ccccc7)cc(-c7ccccc7)cc6c6cc(-c7ccccc7)cc(-c7ccccc7)c65)c(-c5ccccc5)c4)n3)cc2)cc1]

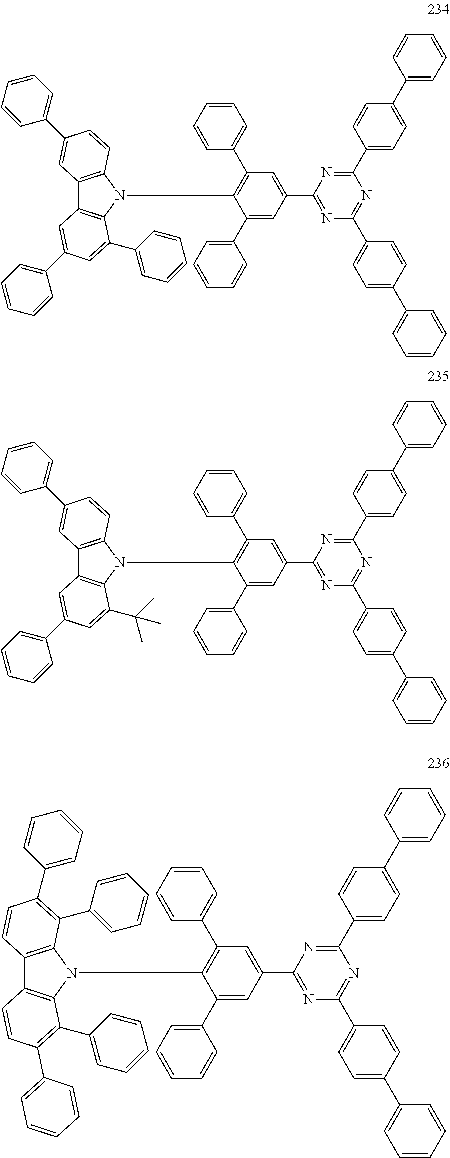 [SMILES: c1ccc(-c2ccc(-c3nc(-c4ccc(-c5ccccc5)cc4)nc(-c4cc(-c5ccccc5)c(-n5c6ccc(-c7ccccc7)cc6c6cc(-c7ccccc7)cc(-c7ccccc7)c65)c(-c5ccccc5)c4)n3)cc2)cc1 ; CC(C)(C)c1cc(-c2ccccc2)cc2c3cc(-c4ccccc4)ccc3n(-c3c(-c4ccccc4)cc(-c4nc(-c5ccc(-c6ccccc6)cc5)nc(-c5ccc(-c6ccccc6)cc5)n4)cc3-c3ccccc3)c12 ; c1ccc(-c2ccc(-c3nc(-c4ccc(-c5ccccc5)cc4)nc(-c4cc(-c5ccccc5)c(-n5c6c(-c7ccccc7)c(-c7ccccc7)ccc6c6ccc(-c7ccccc7)c(-c7ccccc7)c65)c(-c5ccccc5)c4)n3)cc2)cc1]

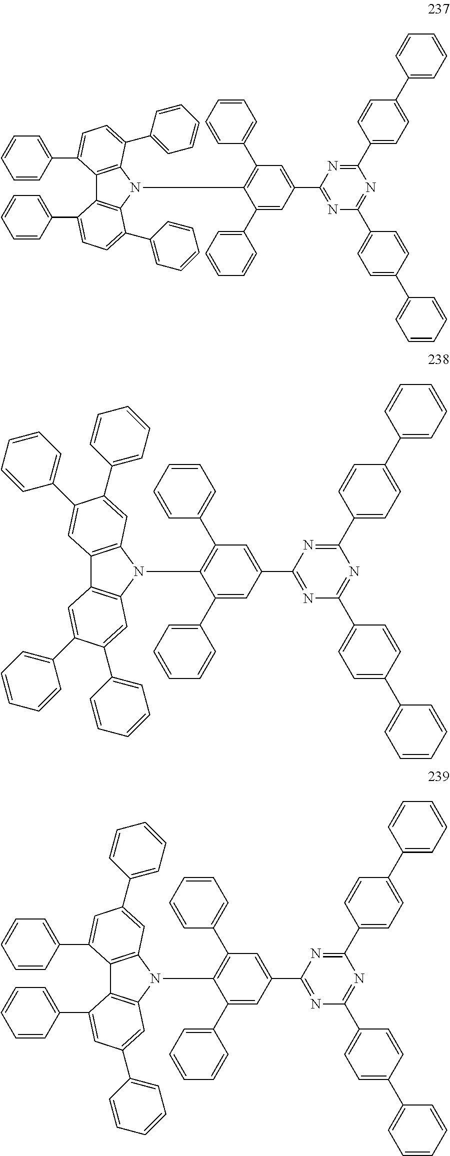 [SMILES: c1ccc(-c2ccc(-c3nc(-c4ccc(-c5ccccc5)cc4)nc(-c4cc(-c5ccccc5)c(-n5c6c(-c7ccccc7)ccc(-c7ccccc7)c6c6c(-c7ccccc7)ccc(-c7ccccc7)c65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6cc(-c7ccccc7)c(-c7ccccc7)cc6c6cc(-c7ccccc7)c(-c7ccccc7)cc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6cc(-c7ccccc7)cc(-c7ccccc7)c6c6c(-c7ccccc7)cc(-c7ccccc7)cc65)c(-c5ccccc5)c4)n3)cc2)cc1]

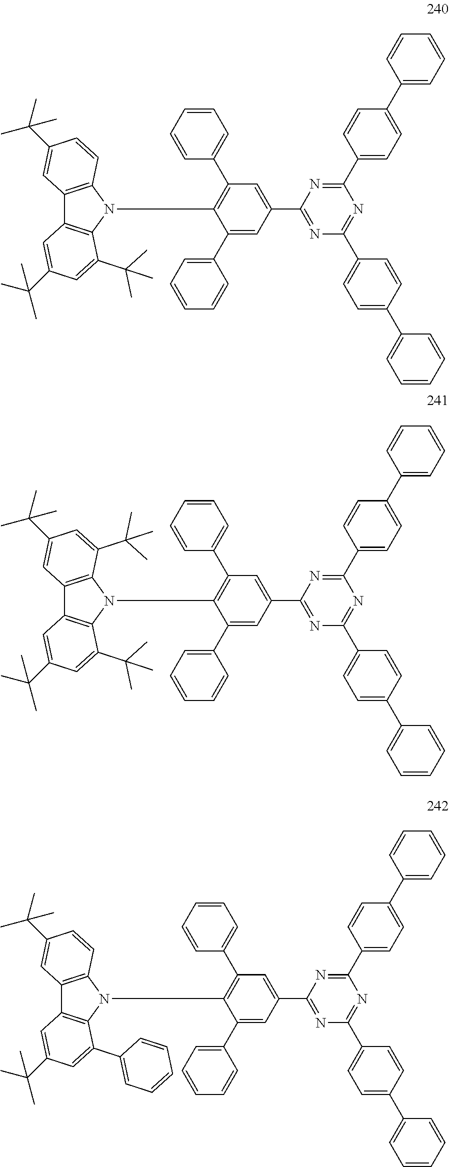 [SMILES: CC(C)(C)c1ccc2c(c1)c1cc(C(C)(C)C)cc(C(C)(C)C)c1n2-c1c(-c2ccccc2)cc(-c2nc(-c3ccc(-c4ccccc4)cc3)nc(-c3ccc(-c4ccccc4)cc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccc(-c4ccccc4)cc3)nc(-c3ccc(-c4ccccc4)cc3)n2)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccc(-c4ccccc4)cc3)nc(-c3ccc(-c4ccccc4)cc3)n2)cc1-c1ccccc1]

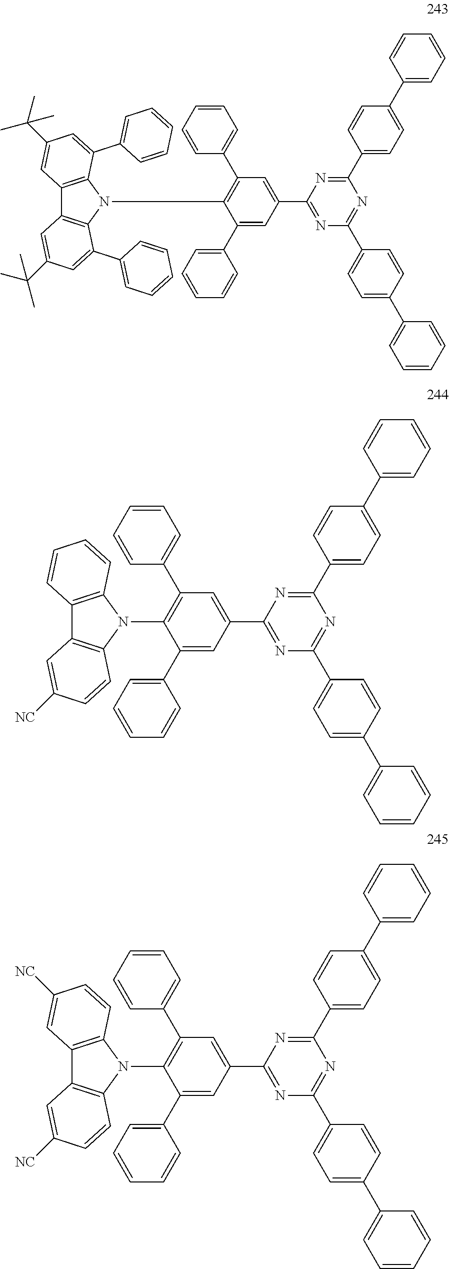

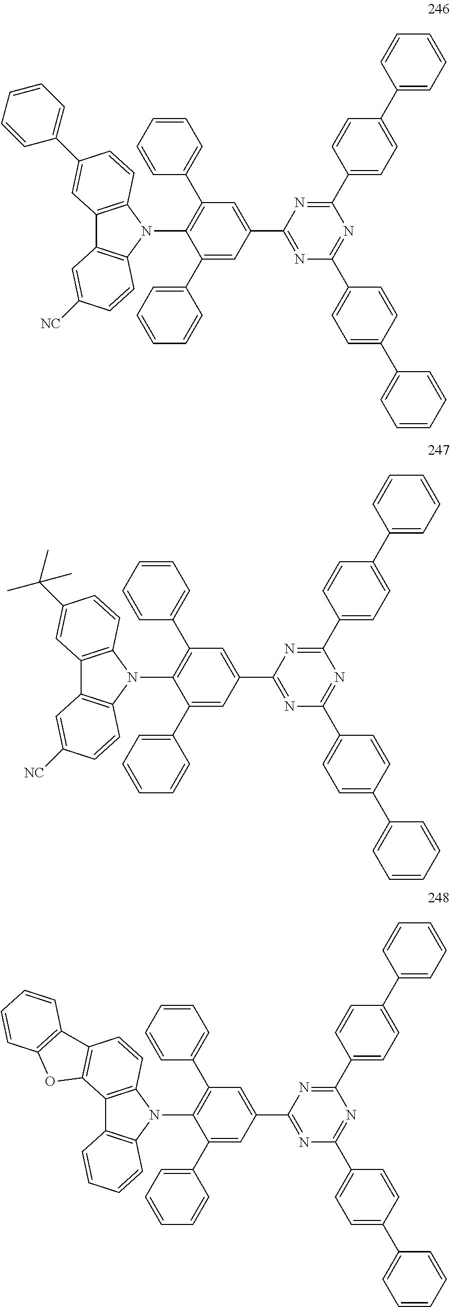

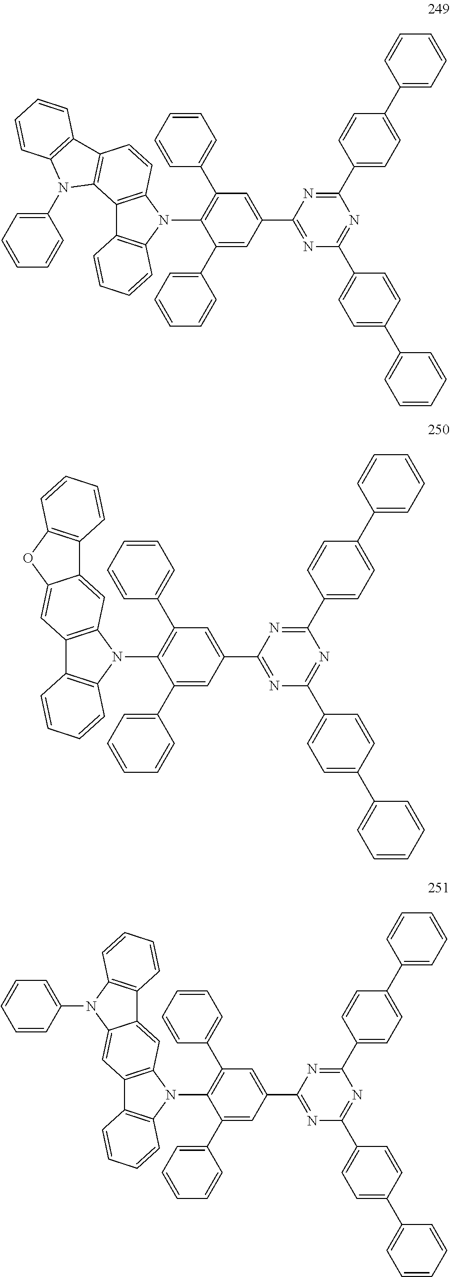 [SMILES: c1ccc(-c2ccc(-c3nc(-c4ccc(-c5ccccc5)cc4)nc(-c4cc(-c5ccccc5)c(-n5c6ccccc6c6c5ccc5c7ccccc7n(-c7ccccc7)c56)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6cc7oc8ccccc8c7cc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6cc7c(cc65)c5ccccc5n7-c5ccccc5)c(-c5ccccc5)c4)n3)cc2)cc1]

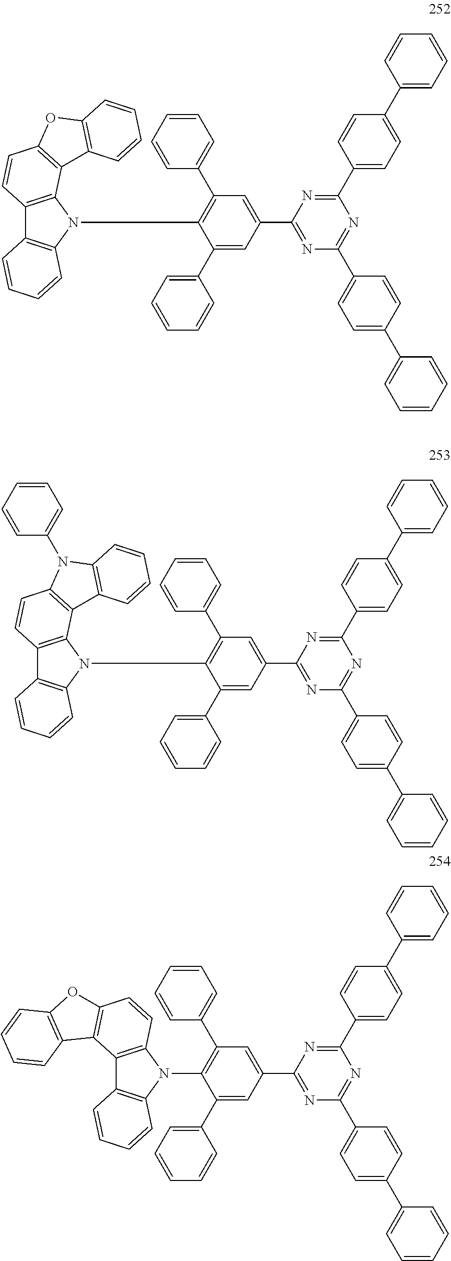

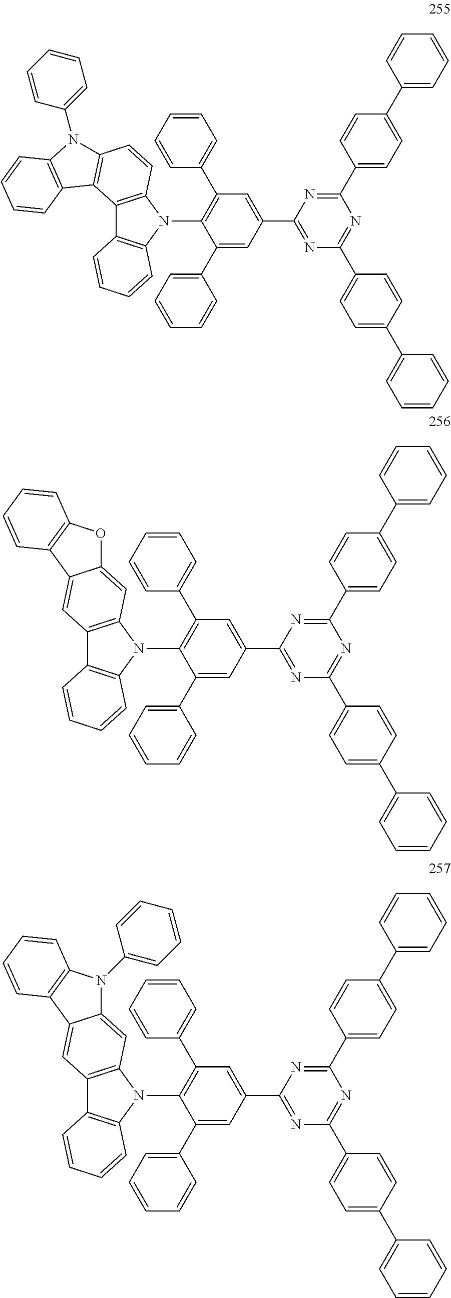

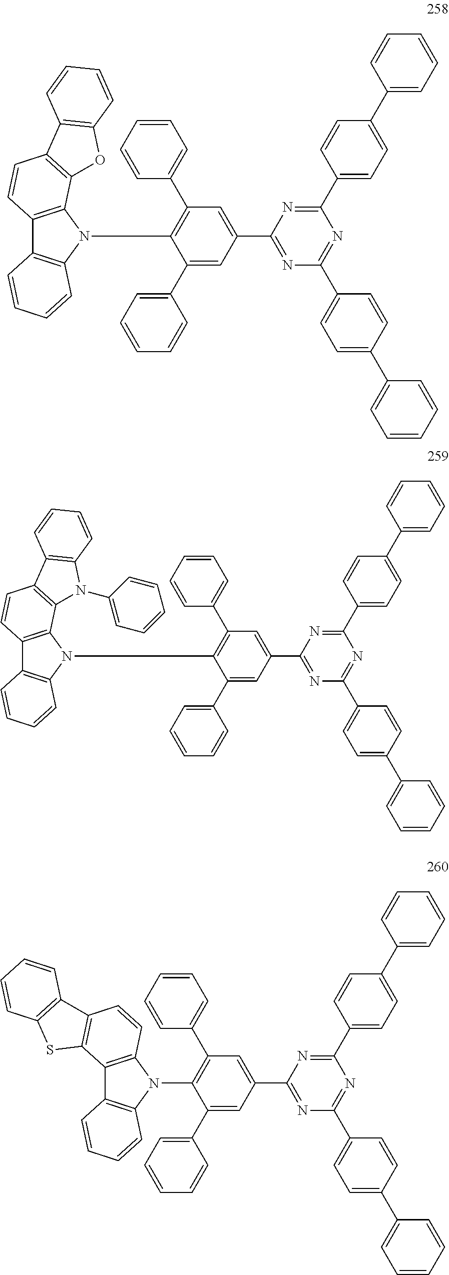

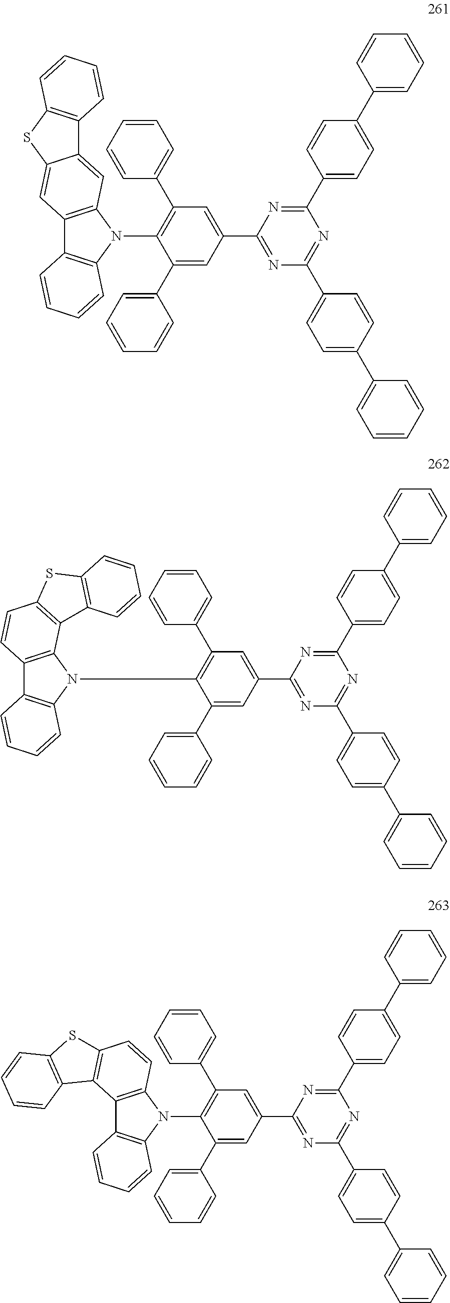 [SMILES: c1ccc(-c2ccc(-c3nc(-c4ccc(-c5ccccc5)cc4)nc(-c4cc(-c5ccccc5)c(-n5c6ccccc6c6cc7sc8ccccc8c7cc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6ccc7sc8ccccc8c7c65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6c7c(ccc65)sc5ccccc57)c(-c5ccccc5)c4)n3)cc2)cc1]

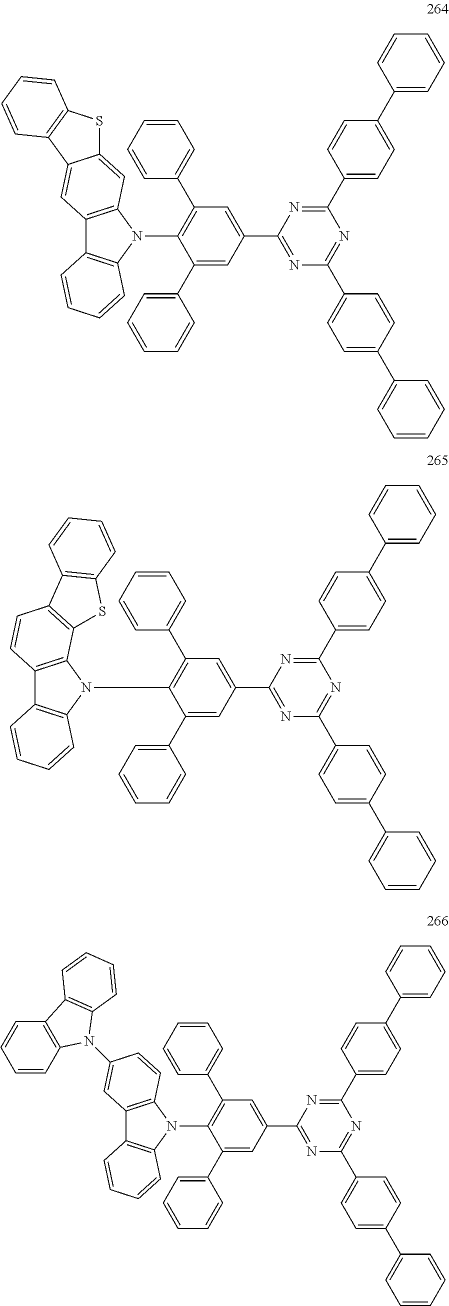 [SMILES: c1ccc(-c2ccc(-c3nc(-c4ccc(-c5ccccc5)cc4)nc(-c4cc(-c5ccccc5)c(-n5c6ccccc6c6cc7c(cc65)sc5ccccc57)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6ccc7c8ccccc8sc7c65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6cc(-n7c8ccccc8c8ccccc87)ccc65)c(-c5ccccc5)c4)n3)cc2)cc1]

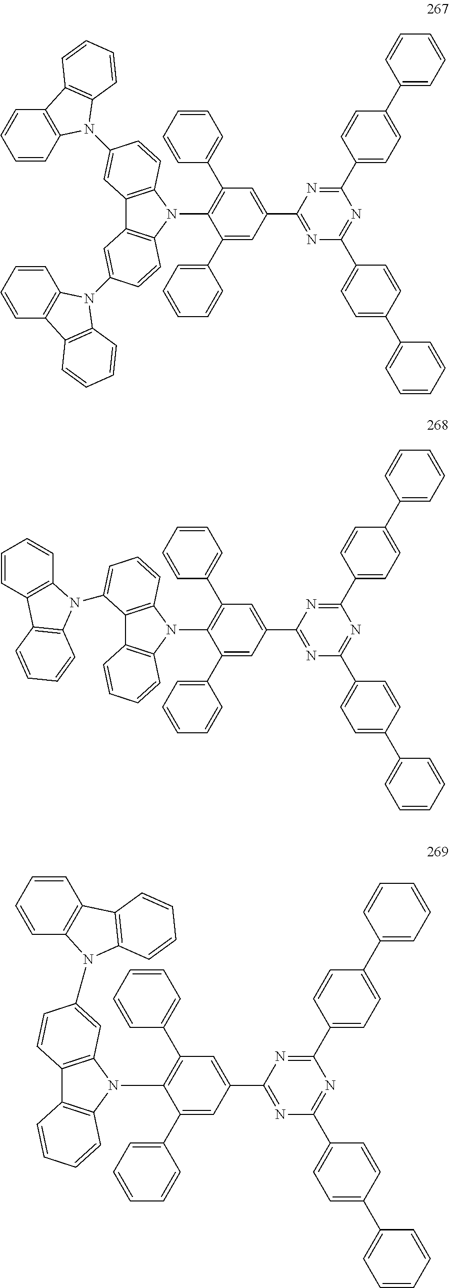 [SMILES: c1ccc(-c2ccc(-c3nc(-c4ccc(-c5ccccc5)cc4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6c(-n7c8ccccc8c8ccccc87)cccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6ccc(-n7c8ccccc8c8ccccc87)cc65)c(-c5ccccc5)c4)n3)cc2)cc1]

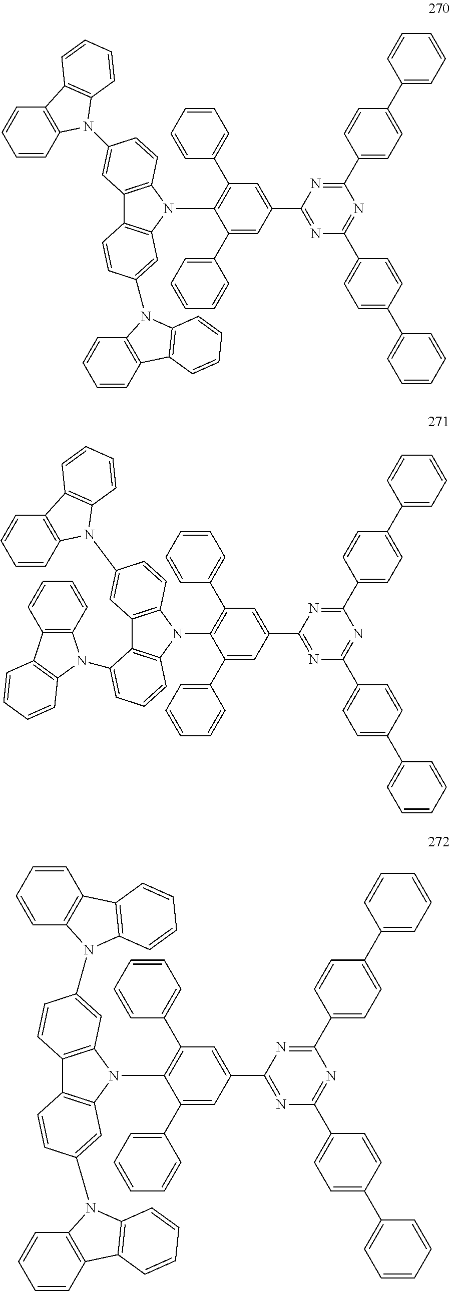 [SMILES: c1ccc(-c2ccc(-c3nc(-c4ccc(-c5ccccc5)cc4)nc(-c4cc(-c5ccccc5)c(-n5c6ccc(-n7c8ccccc8c8ccccc87)cc6c6ccc(-n7c8ccccc8c8ccccc87)cc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccc(-n7c8ccccc8c8ccccc87)cc6c6c(-n7c8ccccc8c8ccccc87)cccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6cc(-n7c8ccccc8c8ccccc87)ccc6c6ccc(-n7c8ccccc8c8ccccc87)cc65)c(-c5ccccc5)c4)n3)cc2)cc1]

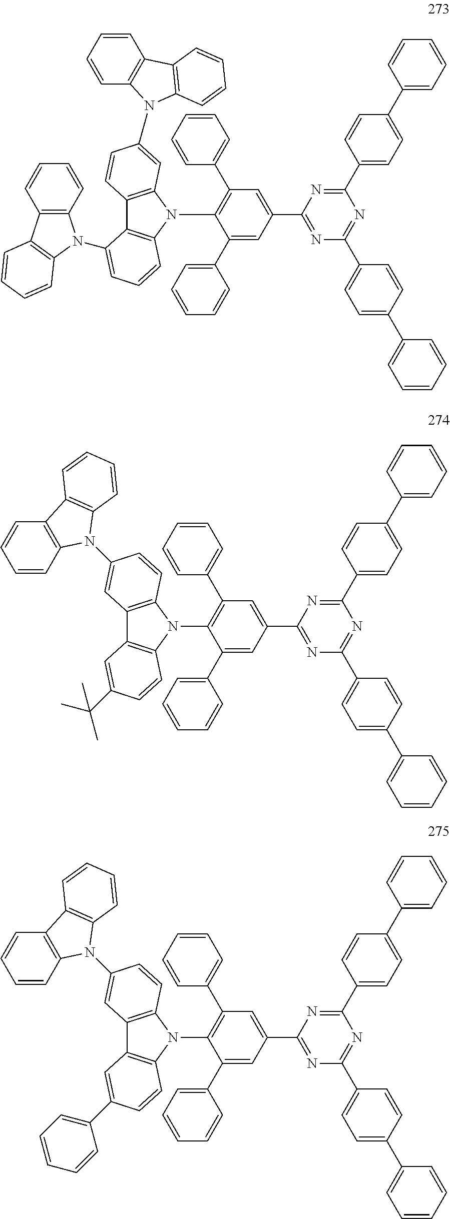 [SMILES: c1ccc(-c2ccc(-c3nc(-c4ccc(-c5ccccc5)cc4)nc(-c4cc(-c5ccccc5)c(-n5c6cc(-n7c8ccccc8c8ccccc87)ccc6c6c(-n7c8ccccc8c8ccccc87)cccc65)c(-c5ccccc5)c4)n3)cc2)cc1 ; CC(C)(C)c1ccc2c(c1)c1cc(-n3c4ccccc4c4ccccc43)ccc1n2-c1c(-c2ccccc2)cc(-c2nc(-c3ccc(-c4ccccc4)cc3)nc(-c3ccc(-c4ccccc4)cc3)n2)cc1-c1ccccc1 ; c1ccc(-c2ccc(-c3nc(-c4ccc(-c5ccccc5)cc4)nc(-c4cc(-c5ccccc5)c(-n5c6ccc(-c7ccccc7)cc6c6cc(-n7c8ccccc8c8ccccc87)ccc65)c(-c5ccccc5)c4)n3)cc2)cc1]

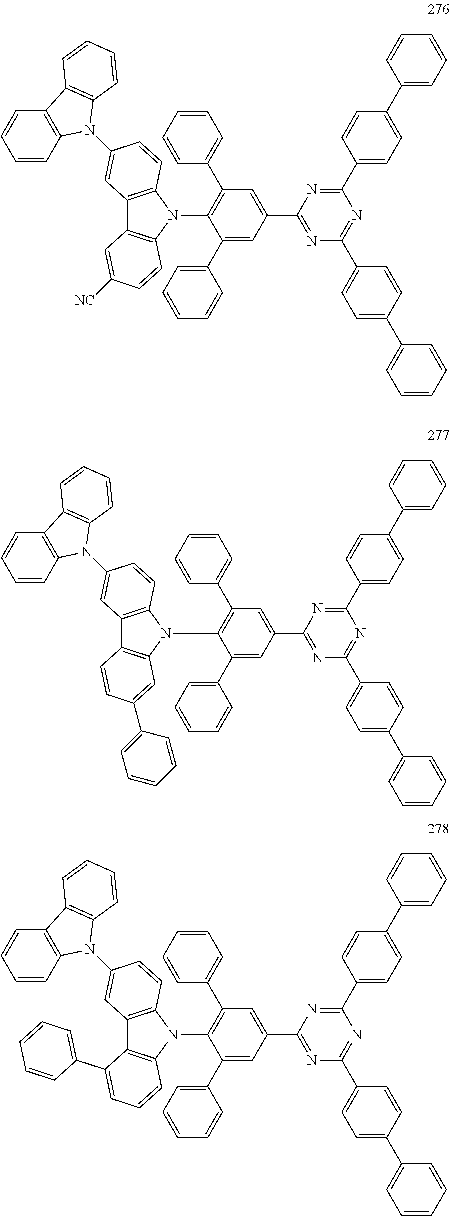 [SMILES: N#Cc1ccc2c(c1)c1cc(-n3c4ccccc4c4ccccc43)ccc1n2-c1c(-c2ccccc2)cc(-c2nc(-c3ccc(-c4ccccc4)cc3)nc(-c3ccc(-c4ccccc4)cc3)n2)cc1-c1ccccc1 ; c1ccc(-c2ccc(-c3nc(-c4ccc(-c5ccccc5)cc4)nc(-c4cc(-c5ccccc5)c(-n5c6ccc(-n7c8ccccc8c8ccccc87)cc6c6ccc(-c7ccccc7)cc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccc(-n7c8ccccc8c8ccccc87)cc6c6c(-c7ccccc7)cccc65)c(-c5ccccc5)c4)n3)cc2)cc1]

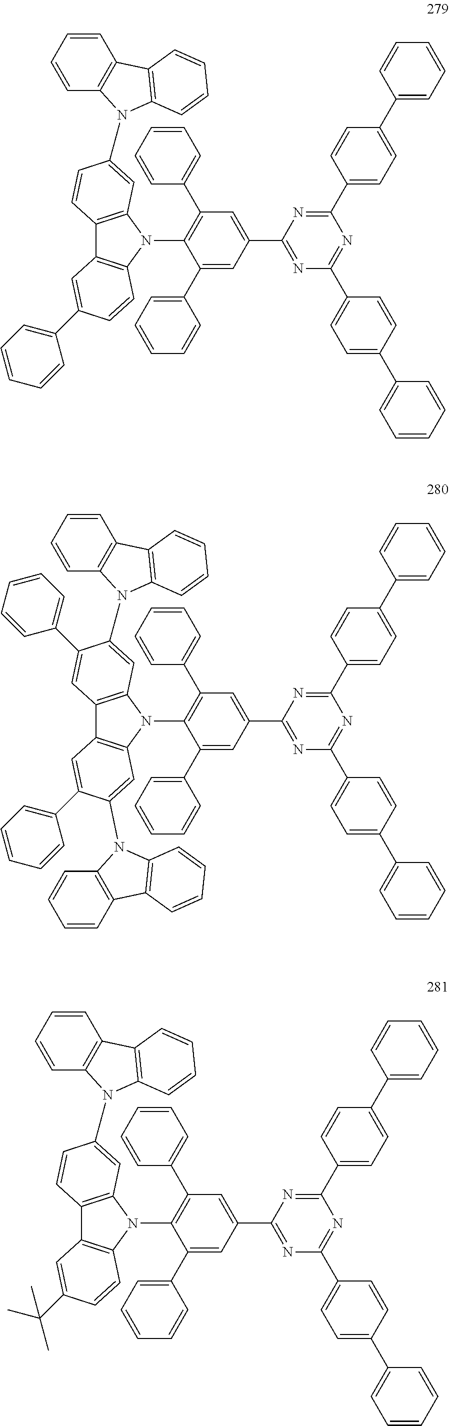 [SMILES: c1ccc(-c2ccc(-c3nc(-c4ccc(-c5ccccc5)cc4)nc(-c4cc(-c5ccccc5)c(-n5c6ccc(-c7ccccc7)cc6c6ccc(-n7c8ccccc8c8ccccc87)cc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6cc(-n7c8ccccc8c8ccccc87)c(-c7ccccc7)cc6c6cc(-c7ccccc7)c(-n7c8ccccc8c8ccccc87)cc65)c(-c5ccccc5)c4)n3)cc2)cc1 ; CC(C)(C)c1ccc2c(c1)c1ccc(-n3c4ccccc4c4ccccc43)cc1n2-c1c(-c2ccccc2)cc(-c2nc(-c3ccc(-c4ccccc4)cc3)nc(-c3ccc(-c4ccccc4)cc3)n2)cc1-c1ccccc1]

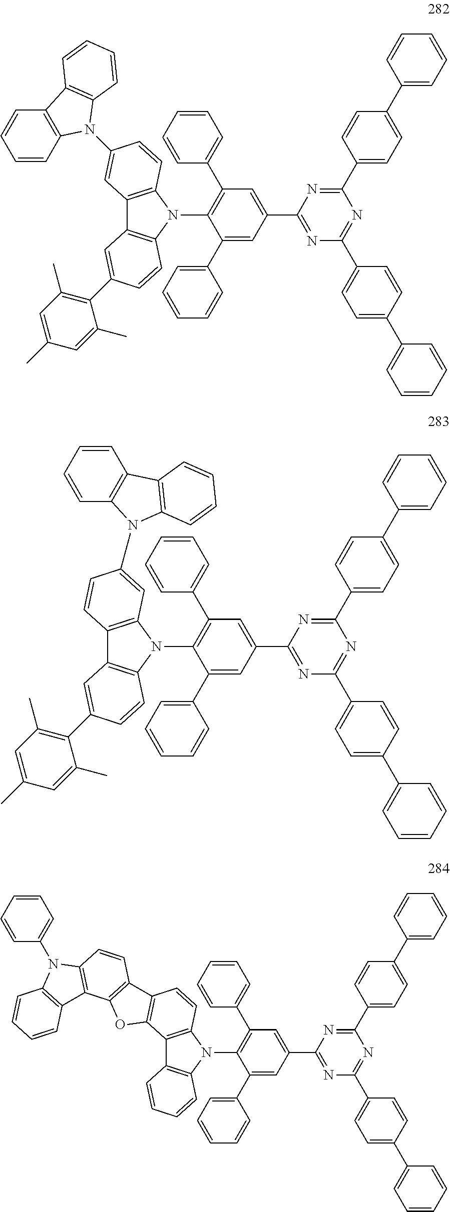 [SMILES: Cc1cc(C)c(-c2ccc3c(c2)c2cc(-n4c5ccccc5c5ccccc54)ccc2n3-c2c(-c3ccccc3)cc(-c3nc(-c4ccc(-c5ccccc5)cc4)nc(-c4ccc(-c5ccccc5)cc4)n3)cc2-c2ccccc2)c(C)c1 ; Cc1cc(C)c(-c2ccc3c(c2)c2ccc(-n4c5ccccc5c5ccccc54)cc2n3-c2c(-c3ccccc3)cc(-c3nc(-c4ccc(-c5ccccc5)cc4)nc(-c4ccc(-c5ccccc5)cc4)n3)cc2-c2ccccc2)c(C)c1 ; c1ccc(-c2ccc(-c3nc(-c4ccc(-c5ccccc5)cc4)nc(-c4cc(-c5ccccc5)c(-n5c6ccccc6c6c7oc8c(ccc9c8c8ccccc8n9-c8ccccc8)c7ccc65)c(-c5ccccc5)c4)n3)cc2)cc1]

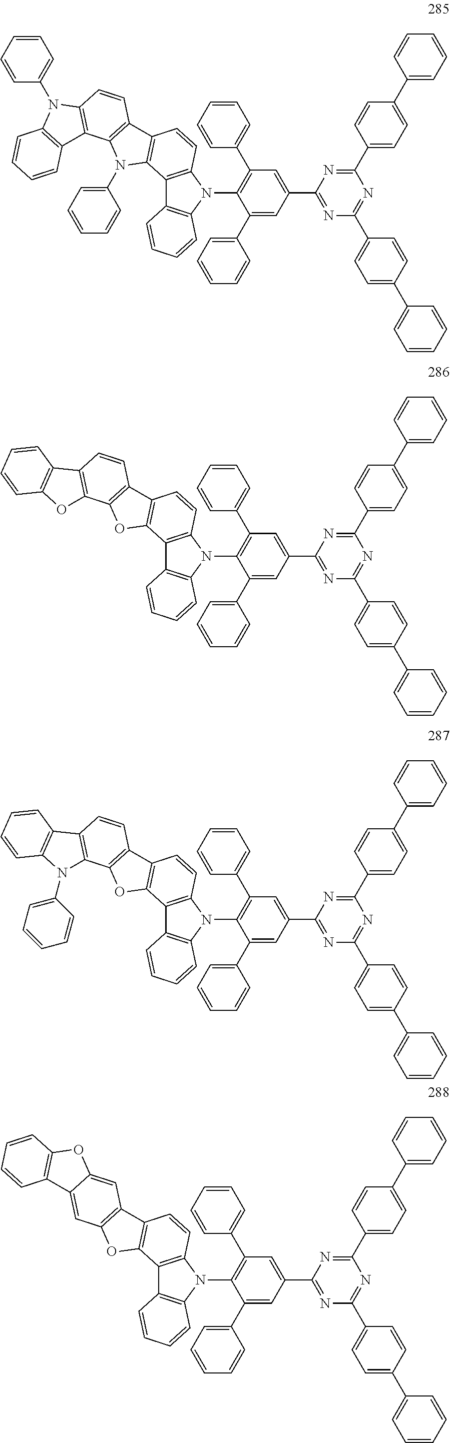

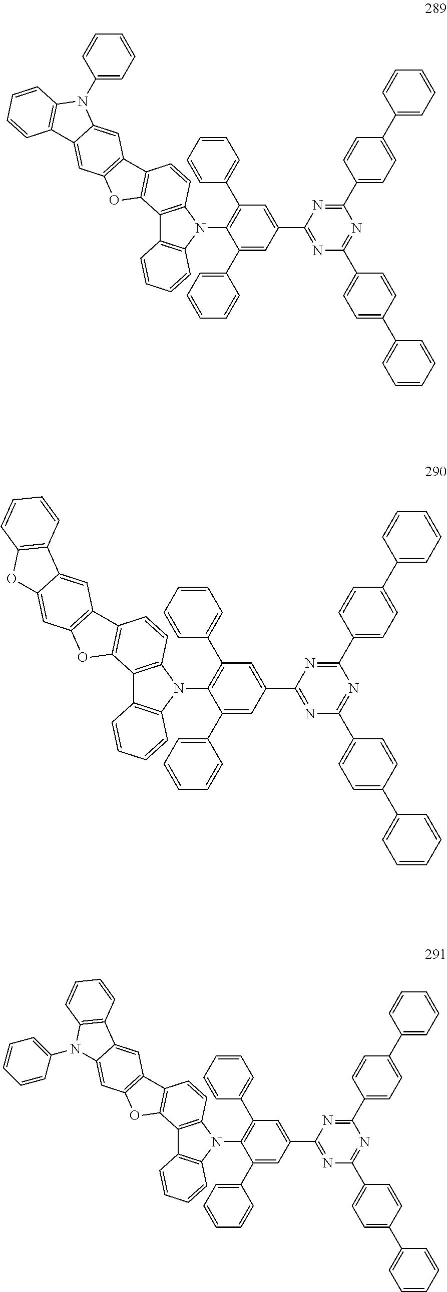 [SMILES: c1ccc(-c2ccc(-c3nc(-c4ccc(-c5ccccc5)cc4)nc(-c4cc(-c5ccccc5)c(-n5c6ccccc6c6c7oc8cc9c%10ccccc%10n(-c%10ccccc%10)c9cc8c7ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6c7oc8cc9oc%10ccccc%10c9cc8c7ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6c7oc8cc9c(cc8c7ccc65)c5ccccc5n9-c5ccccc5)c(-c5ccccc5)c4)n3)cc2)cc1]

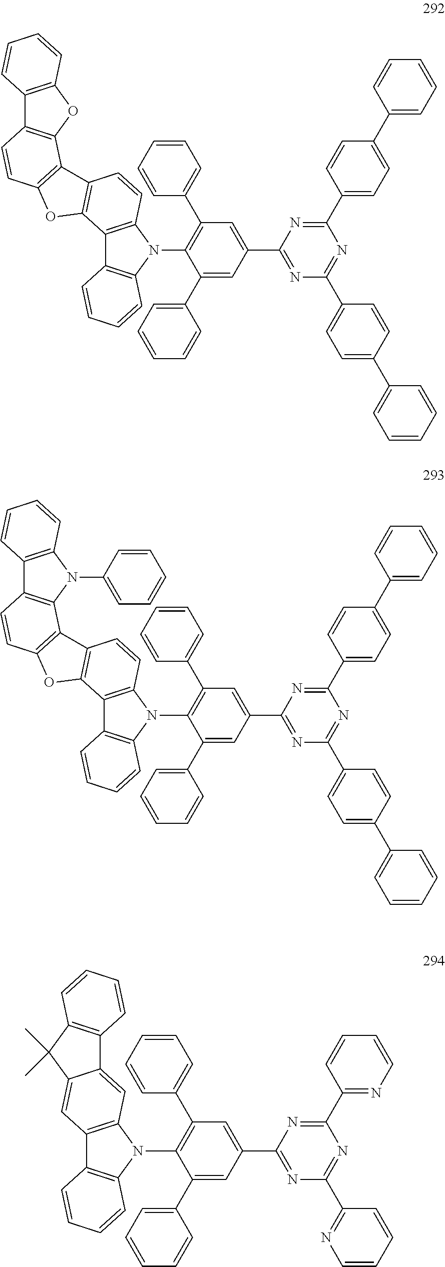 [SMILES: c1ccc(-c2ccc(-c3nc(-c4ccc(-c5ccccc5)cc4)nc(-c4cc(-c5ccccc5)c(-n5c6ccccc6c6c7oc8ccc9c%10ccccc%10oc9c8c7ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6c7oc8ccc9c%10ccccc%10n(-c%10ccccc%10)c9c8c7ccc65)c(-c5ccccc5)c4)n3)cc2)cc1 ; CC1(C)c2ccccc2-c2cc3c(cc21)c1ccccc1n3-c1c(-c2ccccc2)cc(-c2nc(-c3ccccn3)nc(-c3ccccn3)n2)cc1-c1ccccc1]

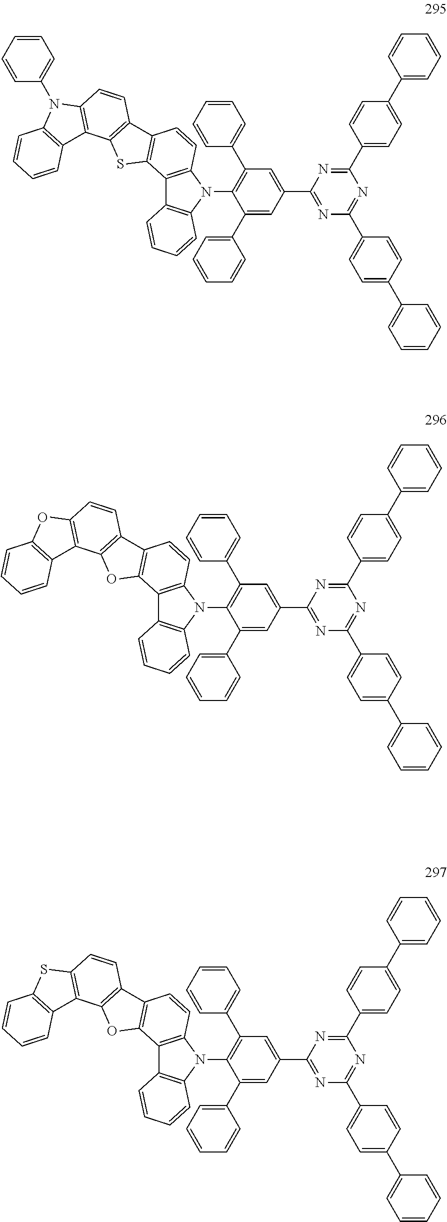 [SMILES: c1ccc(-c2ccc(-c3nc(-c4ccc(-c5ccccc5)cc4)nc(-c4cc(-c5ccccc5)c(-n5c6ccccc6c6c7sc8c(ccc9c8c8ccccc8n9-c8ccccc8)c7ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6c7oc8c(ccc9oc%10ccccc%10c98)c7ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6c7oc8c(ccc9sc%10ccccc%10c98)c7ccc65)c(-c5ccccc5)c4)n3)cc2)cc1]

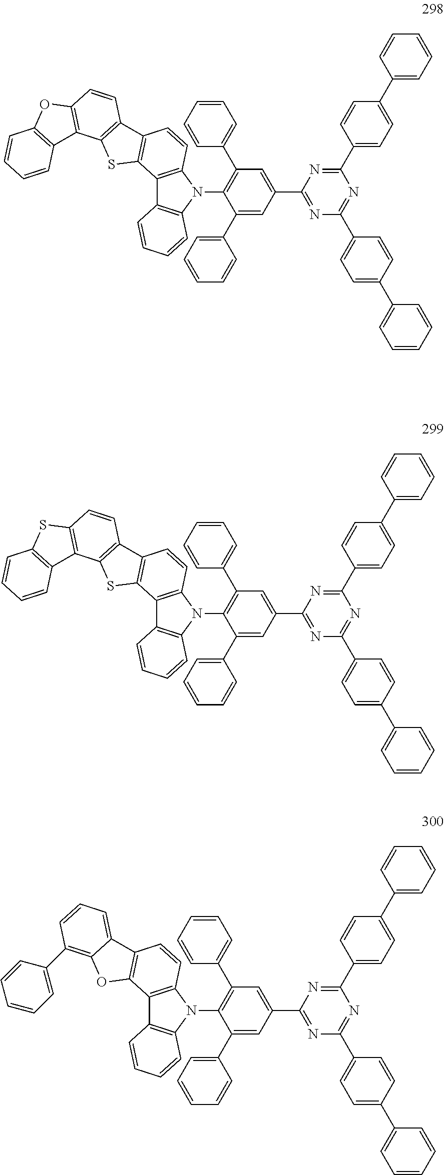 [SMILES: c1ccc(-c2ccc(-c3nc(-c4ccc(-c5ccccc5)cc4)nc(-c4cc(-c5ccccc5)c(-n5c6ccccc6c6c7sc8c(ccc9oc%10ccccc%10c98)c7ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6c7sc8c(ccc9sc%10ccccc%10c98)c7ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6ccccc6c6c7oc8c(-c9ccccc9)cccc8c7ccc65)c(-c5ccccc5)c4)n3)cc2)cc1]

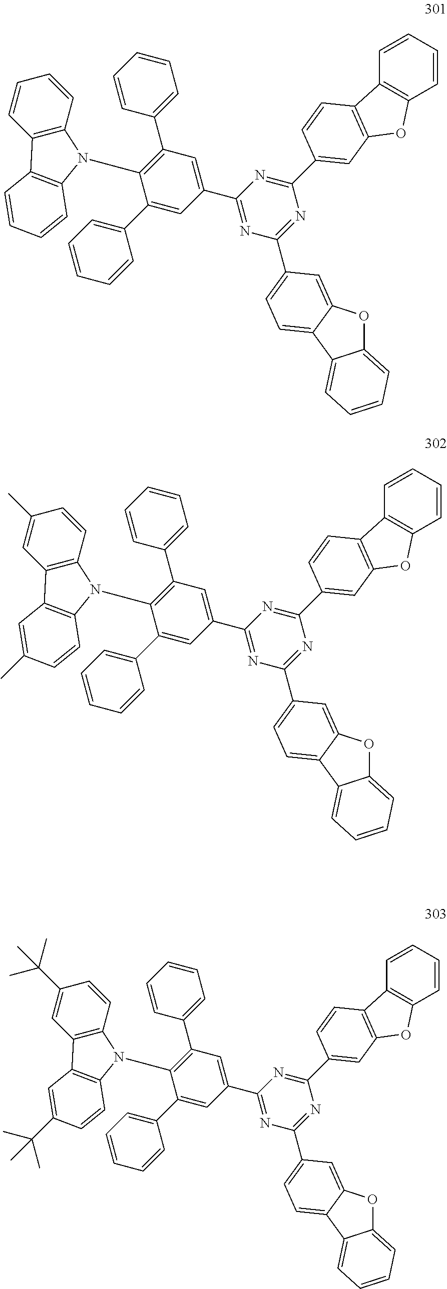 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3ccccc32)cc1 ; Cc1ccc2c(c1)c1cc(C)ccc1n2-c1c(-c2ccccc2)cc(-c2nc(-c3ccc4c(c3)oc3ccccc34)nc(-c3ccc4c(c3)oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3ccc4c(c3)oc3ccccc34)nc(-c3ccc4c(c3)oc3ccccc34)n2)cc1-c1ccccc1]

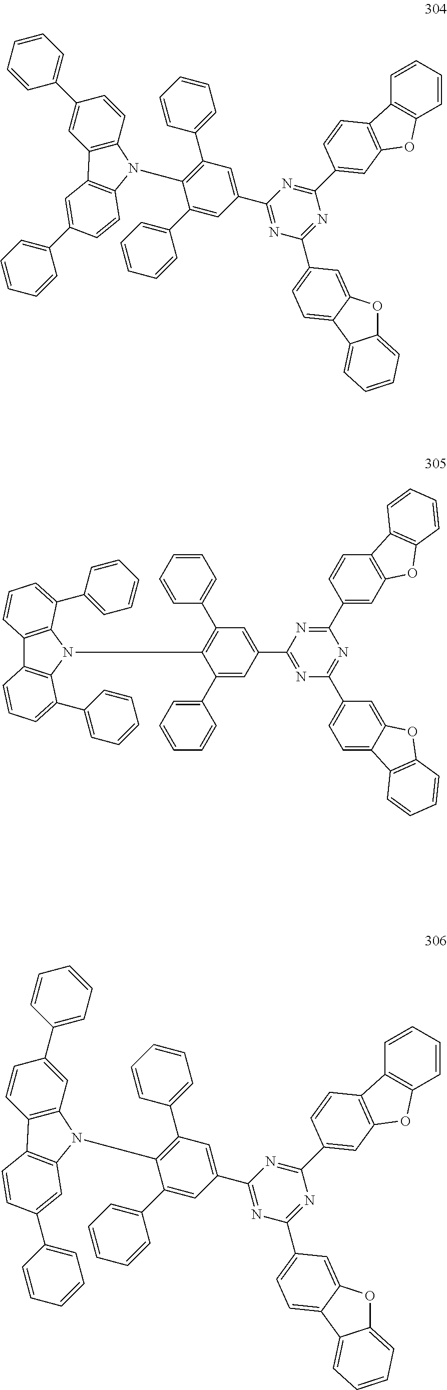 [SMILES: c1ccc(-c2ccc3c(c2)c2cc(-c4ccccc4)ccc2n3-c2c(-c3ccccc3)cc(-c3nc(-c4ccc5c(c4)oc4ccccc45)nc(-c4ccc5c(c4)oc4ccccc45)n3)cc2-c2ccccc2)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3c(-c4ccccc4)cccc3c3cccc(-c4ccccc4)c32)cc1 ; c1ccc(-c2ccc3c4ccc(-c5ccccc5)cc4n(-c4c(-c5ccccc5)cc(-c5nc(-c6ccc7c(c6)oc6ccccc67)nc(-c6ccc7c(c6)oc6ccccc67)n5)cc4-c4ccccc4)c3c2)cc1]

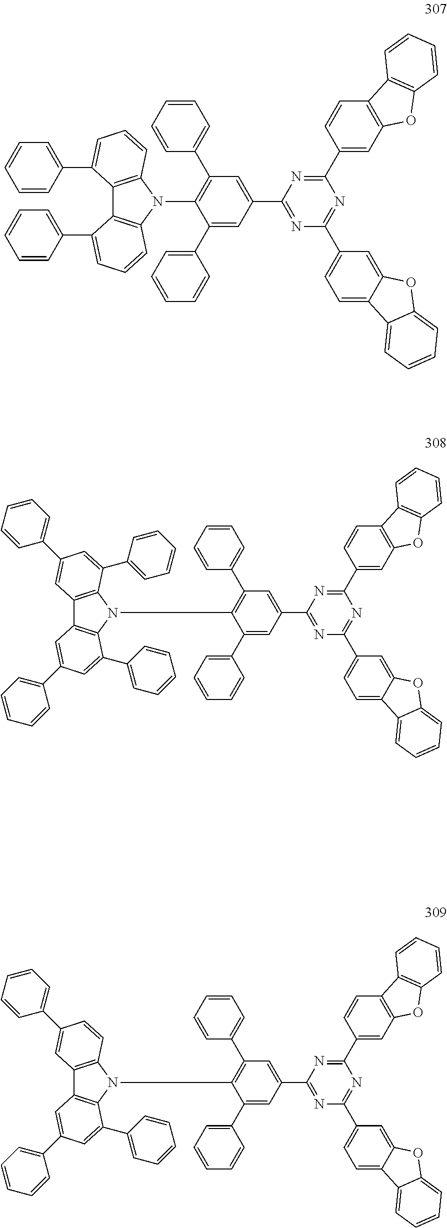 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3cccc(-c4ccccc4)c3c3c(-c4ccccc4)cccc32)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccc5c(c4)oc4ccccc45)nc(-c4ccc5c(c4)oc4ccccc45)n3)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccc5c(c4)oc4ccccc45)nc(-c4ccc5c(c4)oc4ccccc45)n3)cc2-c2ccccc2)cc1]

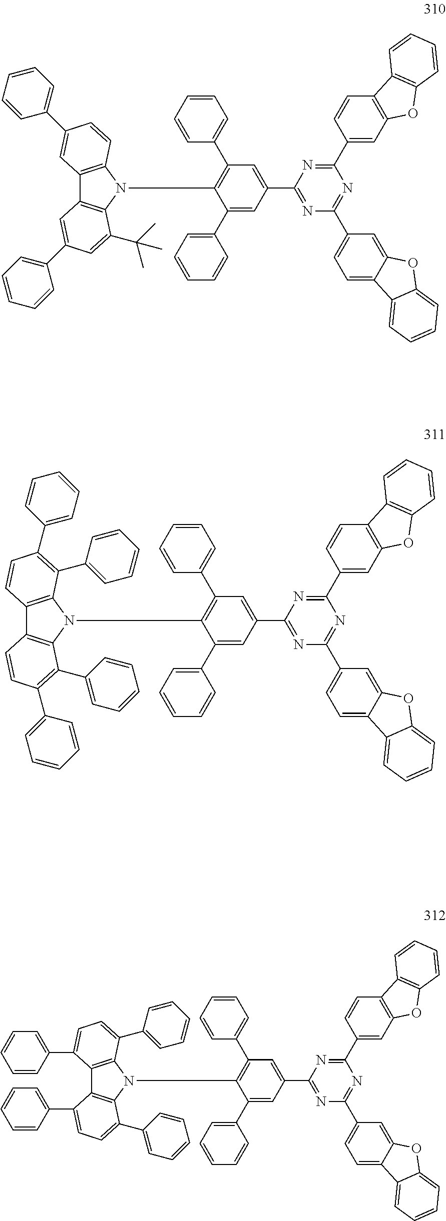 [SMILES: CC(C)(C)c1cc(-c2ccccc2)cc2c3cc(-c4ccccc4)ccc3n(-c3c(-c4ccccc4)cc(-c4nc(-c5ccc6c(c5)oc5ccccc56)nc(-c5ccc6c(c5)oc5ccccc56)n4)cc3-c3ccccc3)c12 ; c1ccc(-c2cc(-c3nc(-c4ccc5c(c4)oc4ccccc45)nc(-c4ccc5c(c4)oc4ccccc45)n3)cc(-c3ccccc3)c2-n2c3c(-c4ccccc4)c(-c4ccccc4)ccc3c3ccc(-c4ccccc4)c(-c4ccccc4)c32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3c(-c4ccccc4)ccc(-c4ccccc4)c3c3c(-c4ccccc4)ccc(-c4ccccc4)c32)cc1]

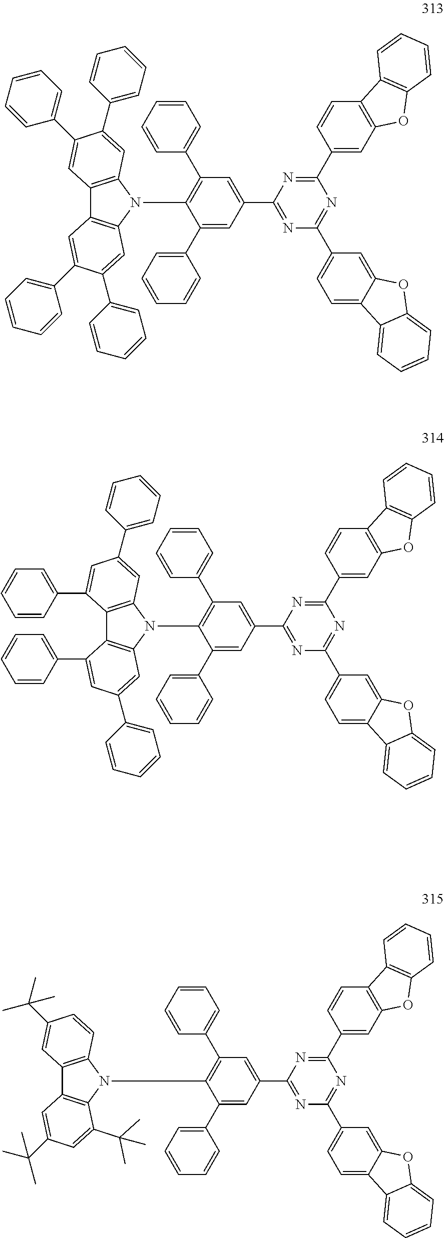 [SMILES: c1ccc(-c2cc3c4cc(-c5ccccc5)c(-c5ccccc5)cc4n(-c4c(-c5ccccc5)cc(-c5nc(-c6ccc7c(c6)oc6ccccc67)nc(-c6ccc7c(c6)oc6ccccc67)n5)cc4-c4ccccc4)c3cc2-c2ccccc2)cc1 ; c1ccc(-c2cc(-c3ccccc3)c3c4c(-c5ccccc5)cc(-c5ccccc5)cc4n(-c4c(-c5ccccc5)cc(-c5nc(-c6ccc7c(c6)oc6ccccc67)nc(-c6ccc7c(c6)oc6ccccc67)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccc4c(c3)oc3ccccc34)nc(-c3ccc4c(c3)oc3ccccc34)n2)cc1-c1ccccc1]

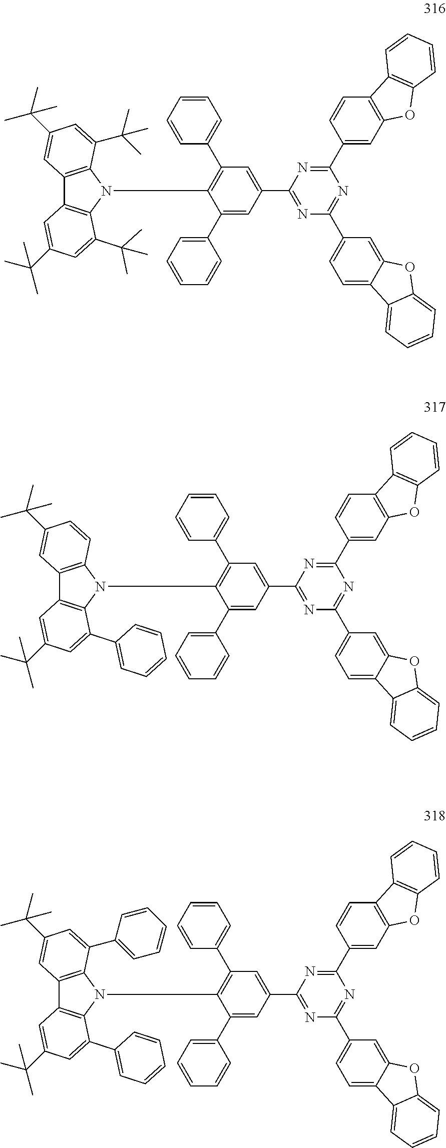 [SMILES: CC(C)(C)c1cc(C(C)(C)C)c2c(c1)c1cc(C(C)(C)C)cc(C(C)(C)C)c1n2-c1c(-c2ccccc2)cc(-c2nc(-c3ccc4c(c3)oc3ccccc34)nc(-c3ccc4c(c3)oc3ccccc34)n2)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccc4c(c3)oc3ccccc34)nc(-c3ccc4c(c3)oc3ccccc34)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccc4c(c3)oc3ccccc34)nc(-c3ccc4c(c3)oc3ccccc34)n2)cc1-c1ccccc1]

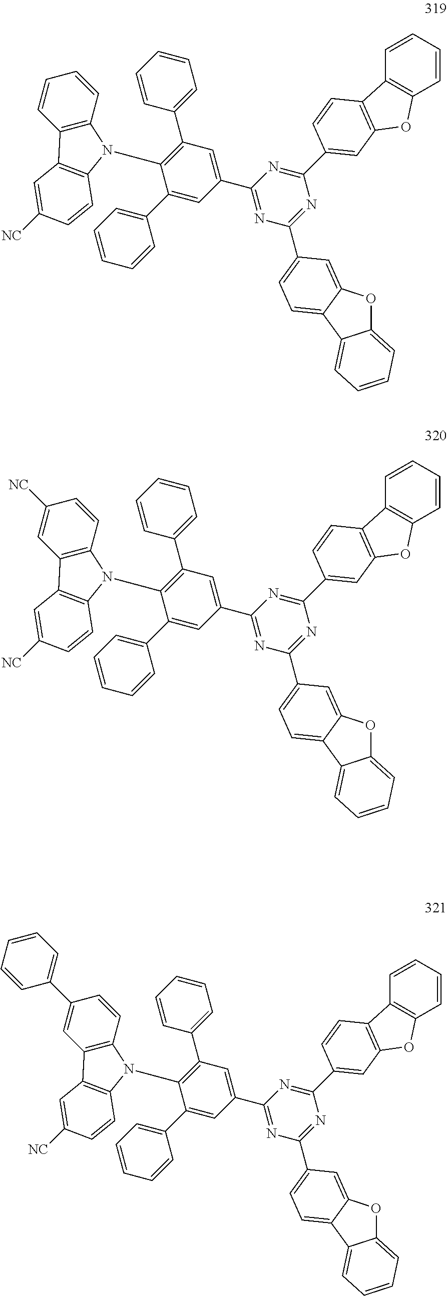 [SMILES: N#Cc1ccc2c(c1)c1ccccc1n2-c1c(-c2ccccc2)cc(-c2nc(-c3ccc4c(c3)oc3ccccc34)nc(-c3ccc4c(c3)oc3ccccc34)n2)cc1-c1ccccc1 ; N#Cc1ccc2c(c1)c1cc(C#N)ccc1n2-c1c(-c2ccccc2)cc(-c2nc(-c3ccc4c(c3)oc3ccccc34)nc(-c3ccc4c(c3)oc3ccccc34)n2)cc1-c1ccccc1 ; N#Cc1ccc2c(c1)c1cc(-c3ccccc3)ccc1n2-c1c(-c2ccccc2)cc(-c2nc(-c3ccc4c(c3)oc3ccccc34)nc(-c3ccc4c(c3)oc3ccccc34)n2)cc1-c1ccccc1]

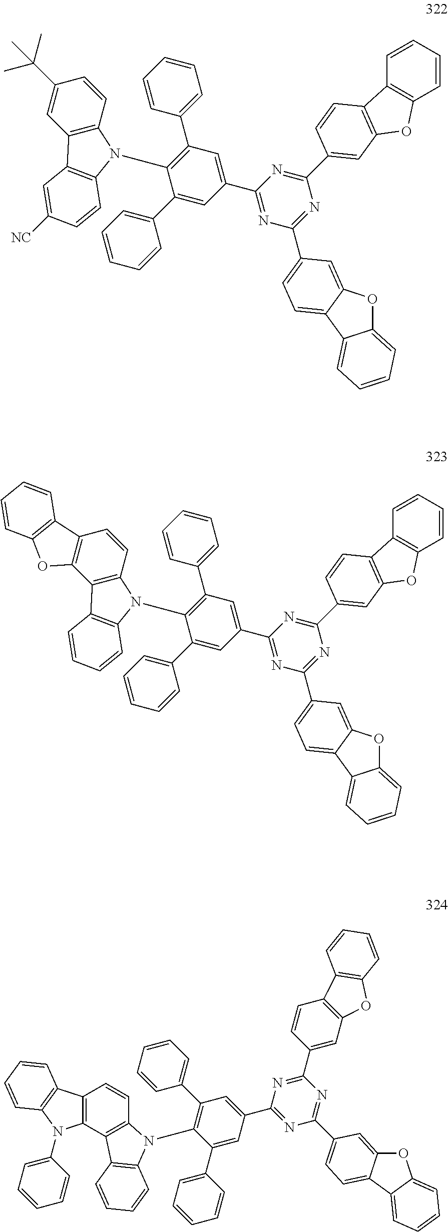 [SMILES: CC(C)(C)c1ccc2c(c1)c1cc(C#N)ccc1n2-c1c(-c2ccccc2)cc(-c2nc(-c3ccc4c(c3)oc3ccccc34)nc(-c3ccc4c(c3)oc3ccccc34)n2)cc1-c1ccccc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5ccccc5c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c2ccc2c4ccccc4n(-c4ccccc4)c23)cc1]

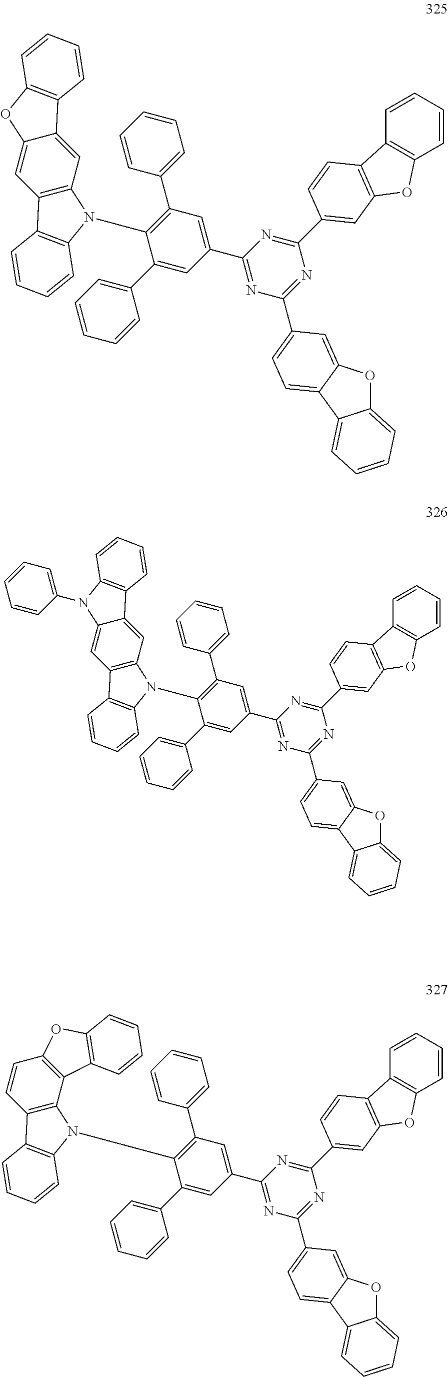 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3cc4oc5ccccc5c4cc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3cc4c(cc32)c2ccccc2n4-c2ccccc2)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3ccc4oc5ccccc5c4c32)cc1]

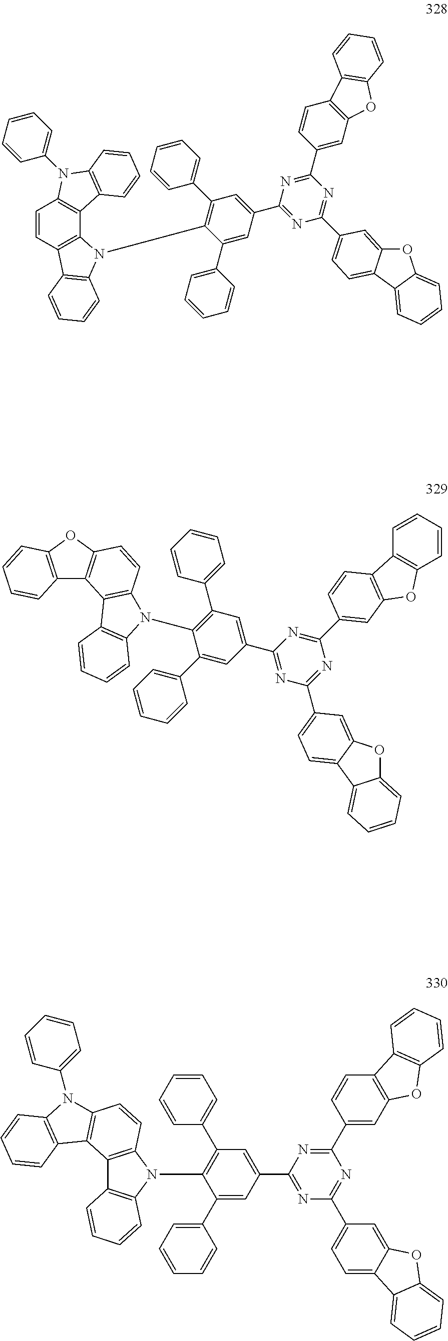 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3ccc4c(c5ccccc5n4-c4ccccc4)c32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4c(ccc32)oc2ccccc24)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4c5ccccc5n(-c5ccccc5)c4ccc32)cc1]

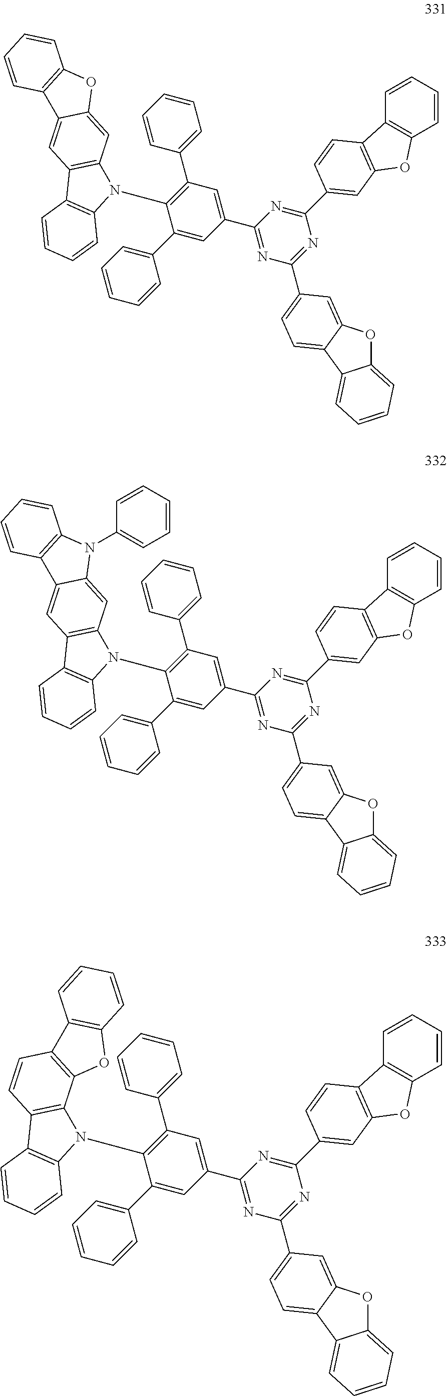 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3cc4c(cc32)oc2ccccc24)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3cc4c5ccccc5n(-c5ccccc5)c4cc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3ccc4c5ccccc5oc4c32)cc1]

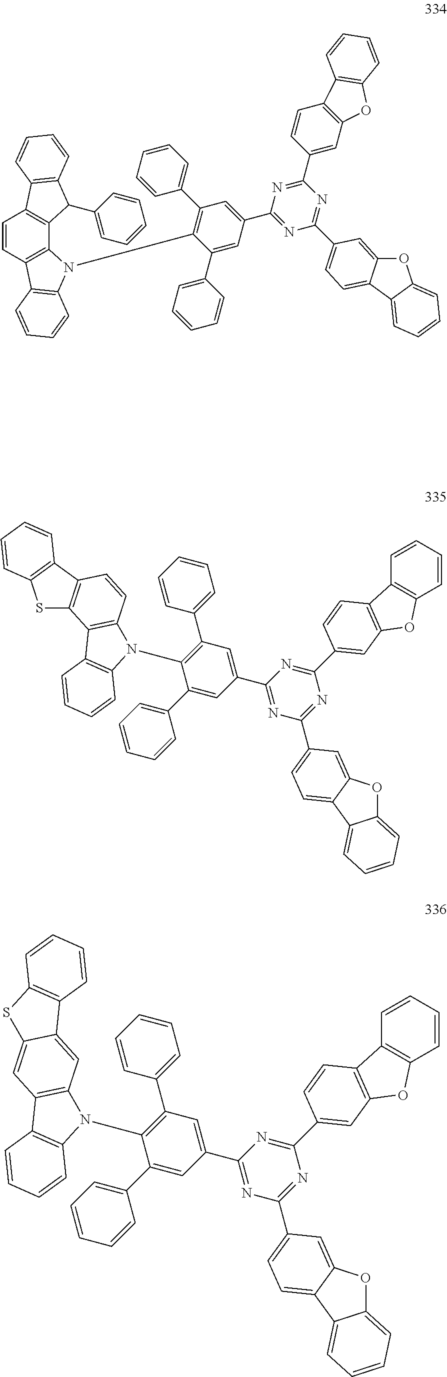 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3ccc4c(c32)C(c2ccccc2)c2ccccc2-4)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4sc5ccccc5c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3cc4sc5ccccc5c4cc32)cc1]

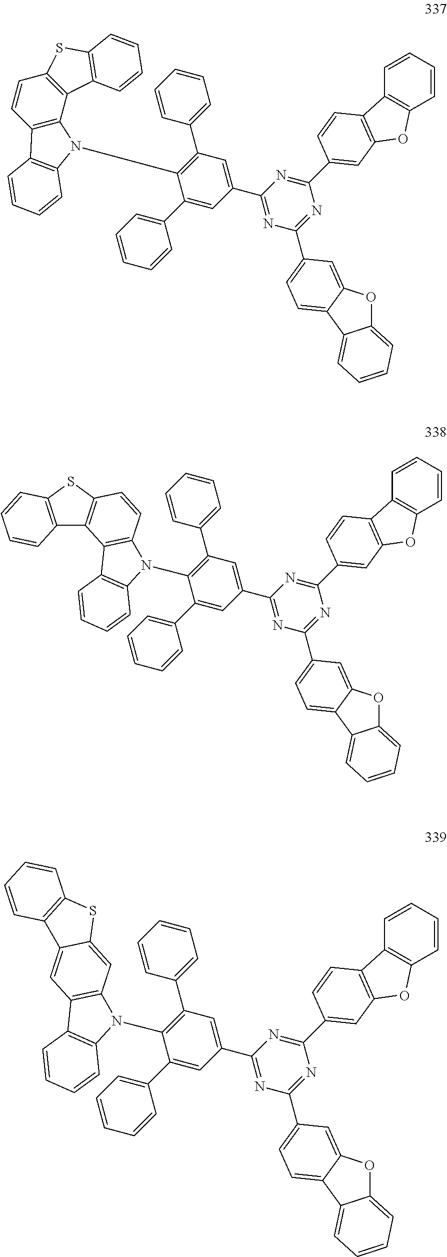 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3ccc4sc5ccccc5c4c32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4c(ccc32)sc2ccccc24)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3cc4c(cc32)sc2ccccc24)cc1]

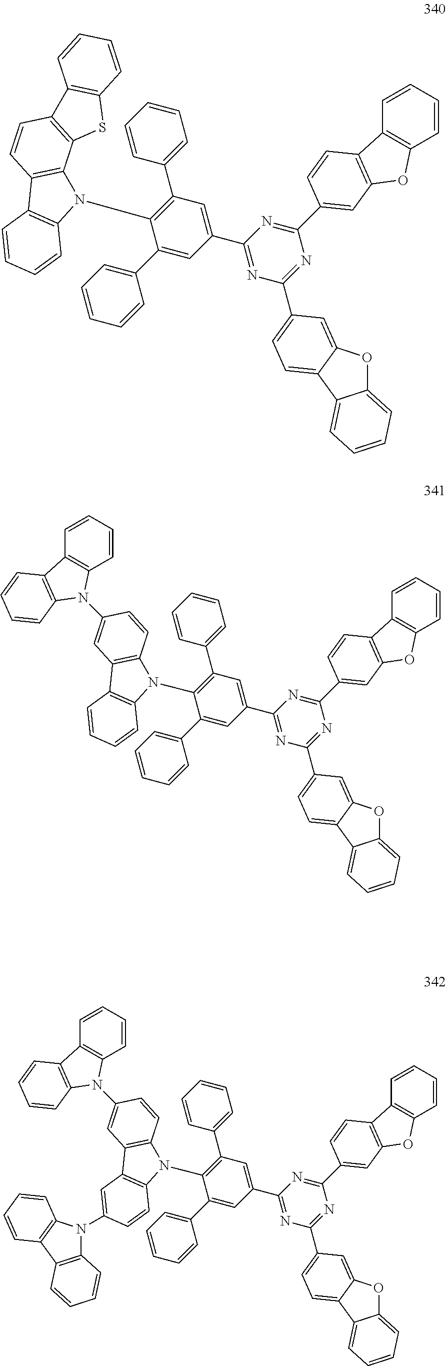 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3ccc4c5ccccc5sc4c32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3cc(-n4c5ccccc5c5ccccc54)ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccc(-n4c5ccccc5c5ccccc54)cc3c3cc(-n4c5ccccc5c5ccccc54)ccc32)cc1]

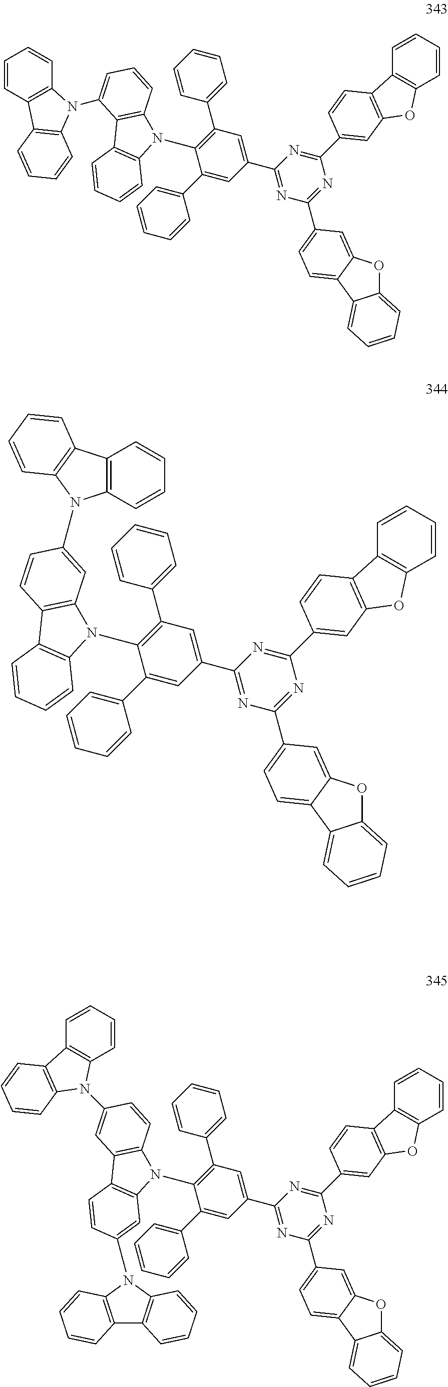 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c(-n4c5ccccc5c5ccccc54)cccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3ccc(-n4c5ccccc5c5ccccc54)cc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccc(-n4c5ccccc5c5ccccc54)cc3c3ccc(-n4c5ccccc5c5ccccc54)cc32)cc1]

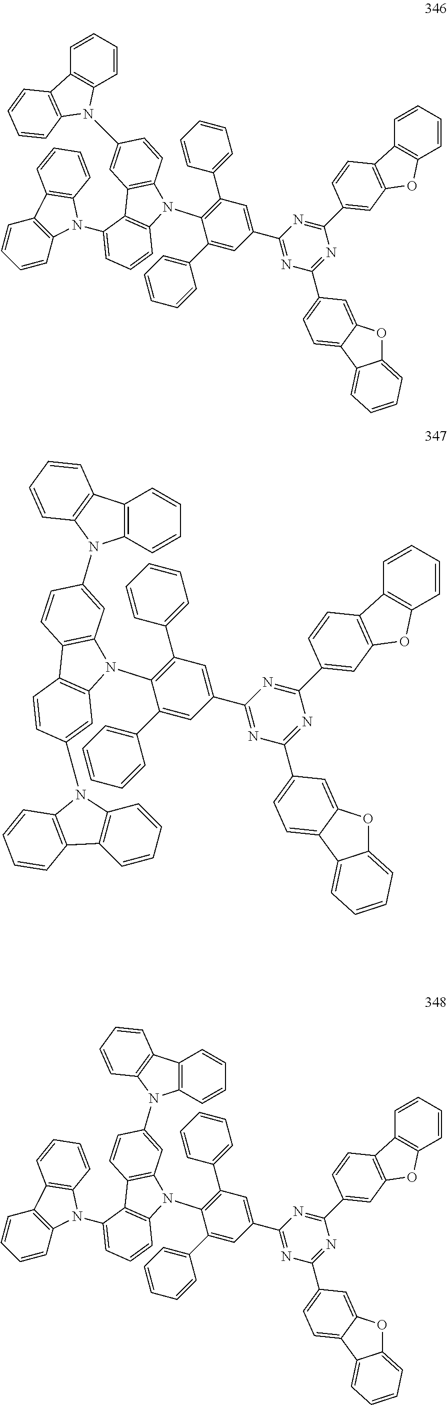 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccc(-n4c5ccccc5c5ccccc54)cc3c3c(-n4c5ccccc5c5ccccc54)cccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3cc(-n4c5ccccc5c5ccccc54)ccc3c3ccc(-n4c5ccccc5c5ccccc54)cc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3cc(-n4c5ccccc5c5ccccc54)ccc3c3c(-n4c5ccccc5c5ccccc54)cccc32)cc1]

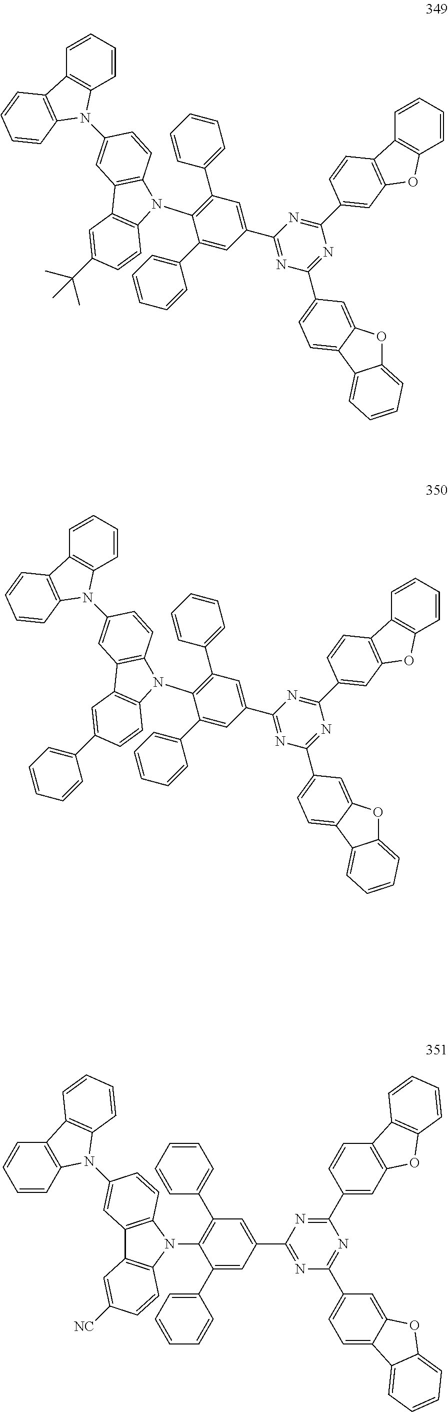 [SMILES: CC(C)(C)c1ccc2c(c1)c1cc(-n3c4ccccc4c4ccccc43)ccc1n2-c1c(-c2ccccc2)cc(-c2nc(-c3ccc4c(c3)oc3ccccc34)nc(-c3ccc4c(c3)oc3ccccc34)n2)cc1-c1ccccc1 ; c1ccc(-c2ccc3c(c2)c2cc(-n4c5ccccc5c5ccccc54)ccc2n3-c2c(-c3ccccc3)cc(-c3nc(-c4ccc5c(c4)oc4ccccc45)nc(-c4ccc5c(c4)oc4ccccc45)n3)cc2-c2ccccc2)cc1 ; N#Cc1ccc2c(c1)c1cc(-n3c4ccccc4c4ccccc43)ccc1n2-c1c(-c2ccccc2)cc(-c2nc(-c3ccc4c(c3)oc3ccccc34)nc(-c3ccc4c(c3)oc3ccccc34)n2)cc1-c1ccccc1]

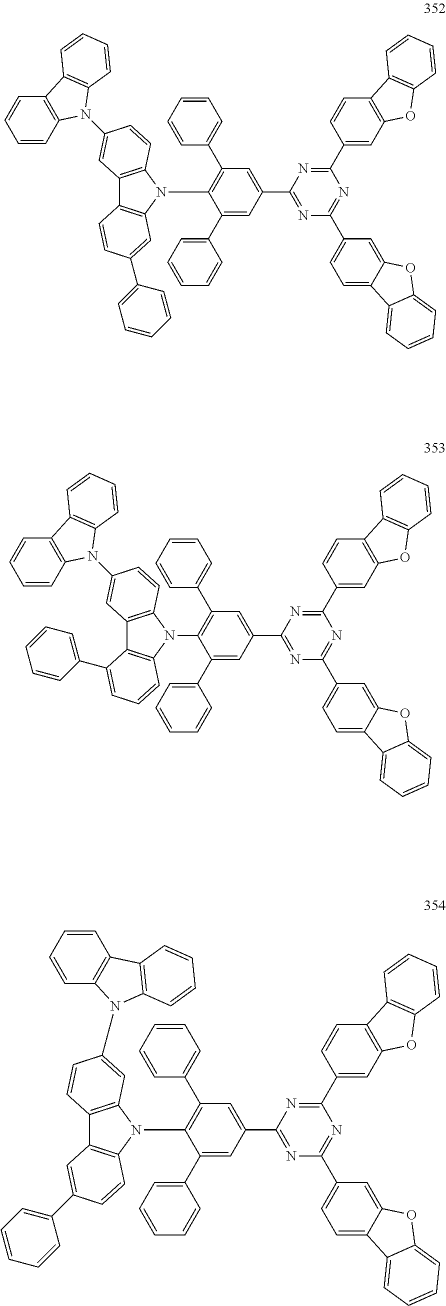 [SMILES: c1ccc(-c2ccc3c4cc(-n5c6ccccc6c6ccccc65)ccc4n(-c4c(-c5ccccc5)cc(-c5nc(-c6ccc7c(c6)oc6ccccc67)nc(-c6ccc7c(c6)oc6ccccc67)n5)cc4-c4ccccc4)c3c2)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccc(-n4c5ccccc5c5ccccc54)cc3c3c(-c4ccccc4)cccc32)cc1 ; c1ccc(-c2ccc3c(c2)c2ccc(-n4c5ccccc5c5ccccc54)cc2n3-c2c(-c3ccccc3)cc(-c3nc(-c4ccc5c(c4)oc4ccccc45)nc(-c4ccc5c(c4)oc4ccccc45)n3)cc2-c2ccccc2)cc1]

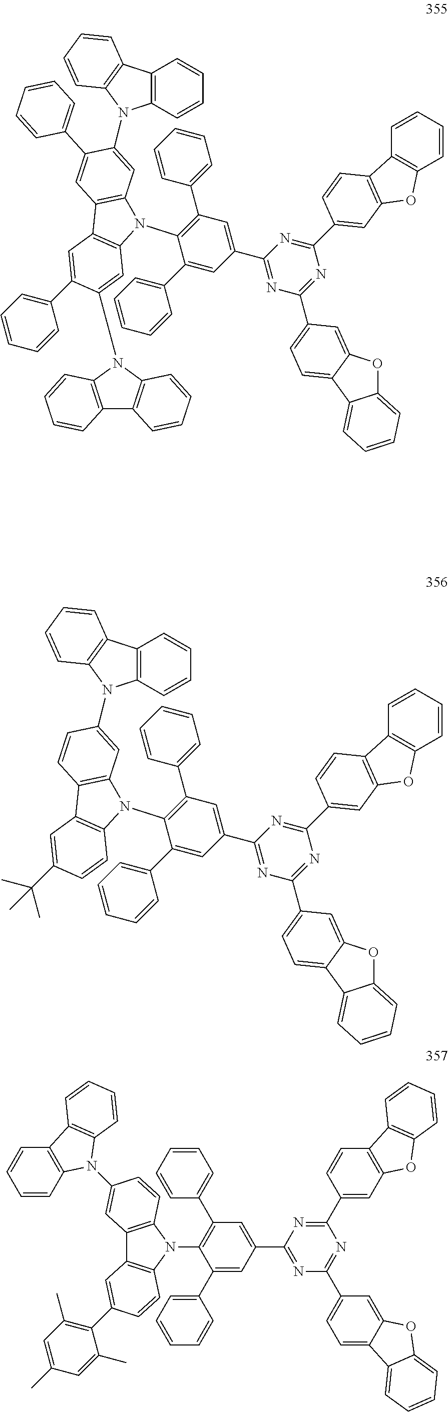 [SMILES: c1ccc(-c2cc3c4cc(-c5ccccc5)c(-n5c6ccccc6c6ccccc65)cc4n(-c4c(-c5ccccc5)cc(-c5nc(-c6ccc7c(c6)oc6ccccc67)nc(-c6ccc7c(c6)oc6ccccc67)n5)cc4-c4ccccc4)c3cc2-n2c3ccccc3c3ccccc32)cc1 ; CC(C)(C)c1ccc2c(c1)c1ccc(-n3c4ccccc4c4ccccc43)cc1n2-c1c(-c2ccccc2)cc(-c2nc(-c3ccc4c(c3)oc3ccccc34)nc(-c3ccc4c(c3)oc3ccccc34)n2)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccc5c(c4)oc4ccccc45)nc(-c4ccc5c(c4)oc4ccccc45)n3)cc2-c2ccccc2)c(C)c1]

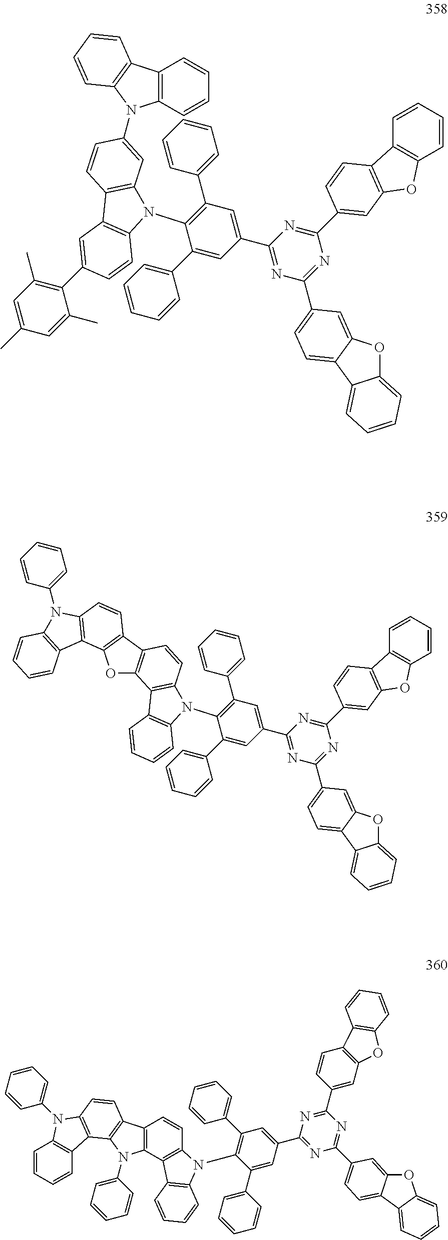 [SMILES: Cc1cc(C)c(-c2ccc3c(c2)c2ccc(-n4c5ccccc5c5ccccc54)cc2n3-c2c(-c3ccccc3)cc(-c3nc(-c4ccc5c(c4)oc4ccccc45)nc(-c4ccc5c(c4)oc4ccccc45)n3)cc2-c2ccccc2)c(C)c1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5c(ccc6c5c5ccccc5n6-c5ccccc5)c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c2ccc2c4ccc5c(c6ccccc6n5-c5ccccc5)c4n(-c4ccccc4)c23)cc1]

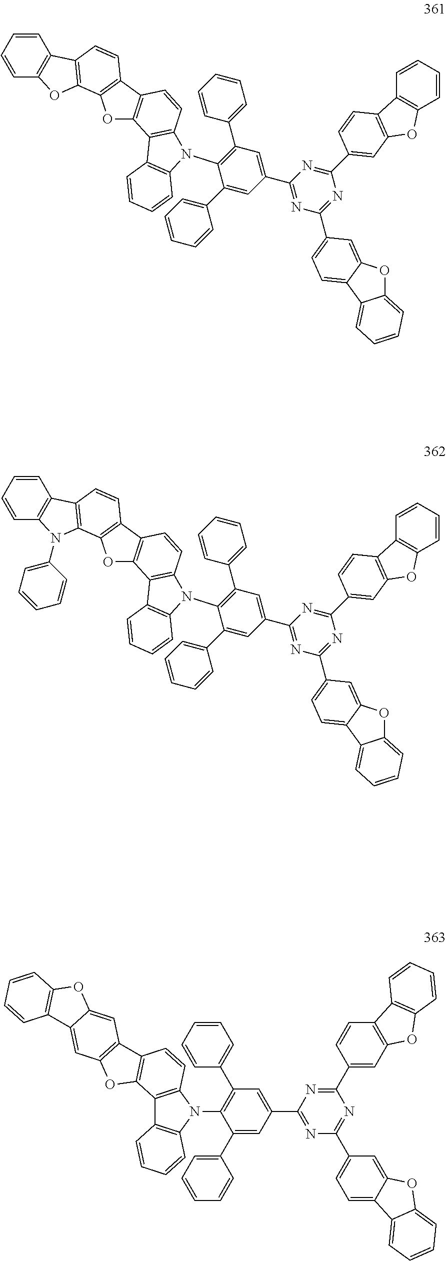 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5c(ccc6c7ccccc7oc65)c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5c(ccc6c7ccccc7n(-c7ccccc7)c65)c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5cc6c(cc5c4ccc32)oc2ccccc26)cc1]

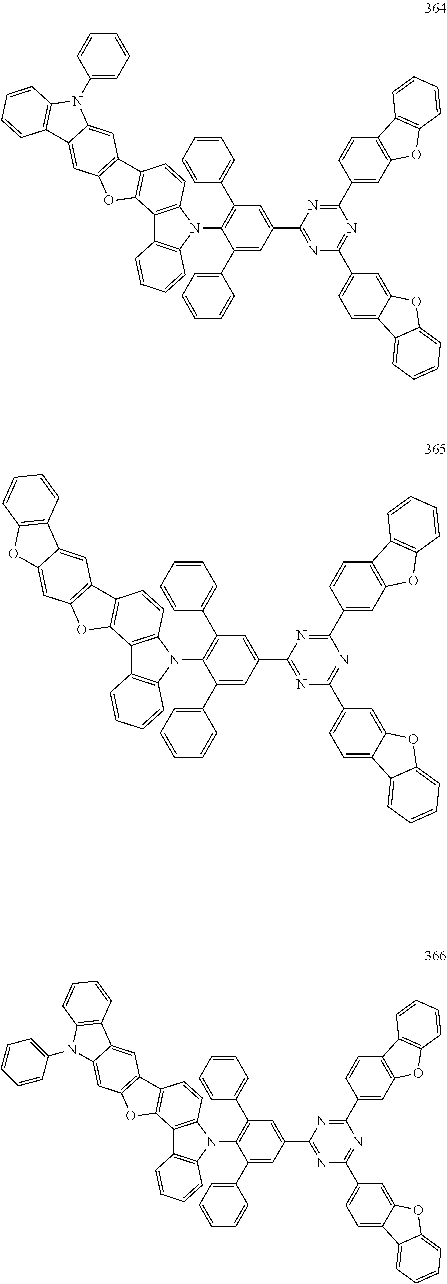 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5cc6c7ccccc7n(-c7ccccc7)c6cc5c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5cc6oc7ccccc7c6cc5c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5cc6c(cc5c4ccc32)c2ccccc2n6-c2ccccc2)cc1]

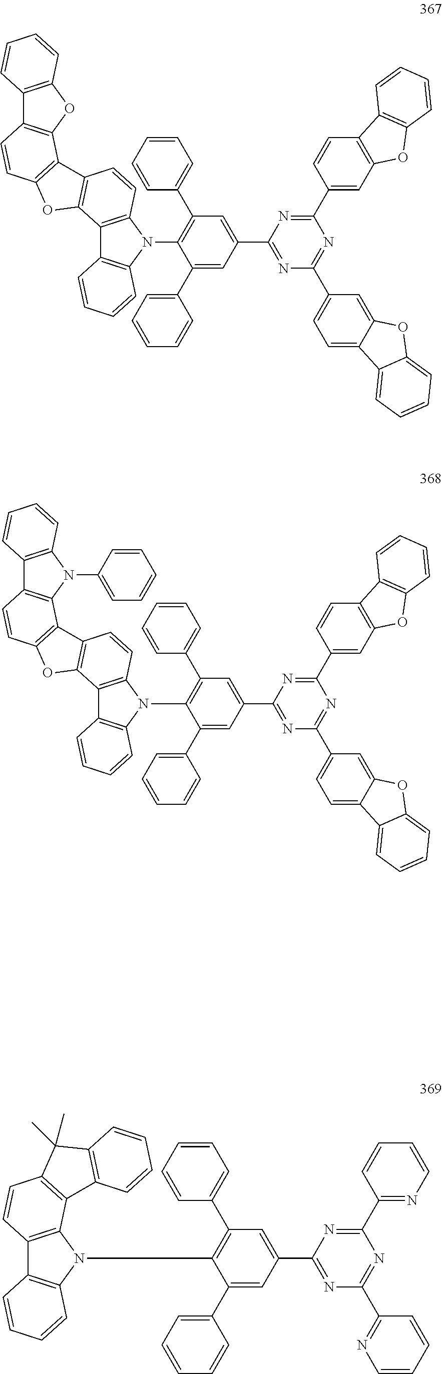 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5ccc6c7ccccc7oc6c5c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5ccc6c7ccccc7n(-c7ccccc7)c6c5c4ccc32)cc1 ; CC1(C)c2ccccc2-c2c1ccc1c3ccccc3n(-c3c(-c4ccccc4)cc(-c4nc(-c5ccccn5)nc(-c5ccccn5)n4)cc3-c3ccccc3)c21]

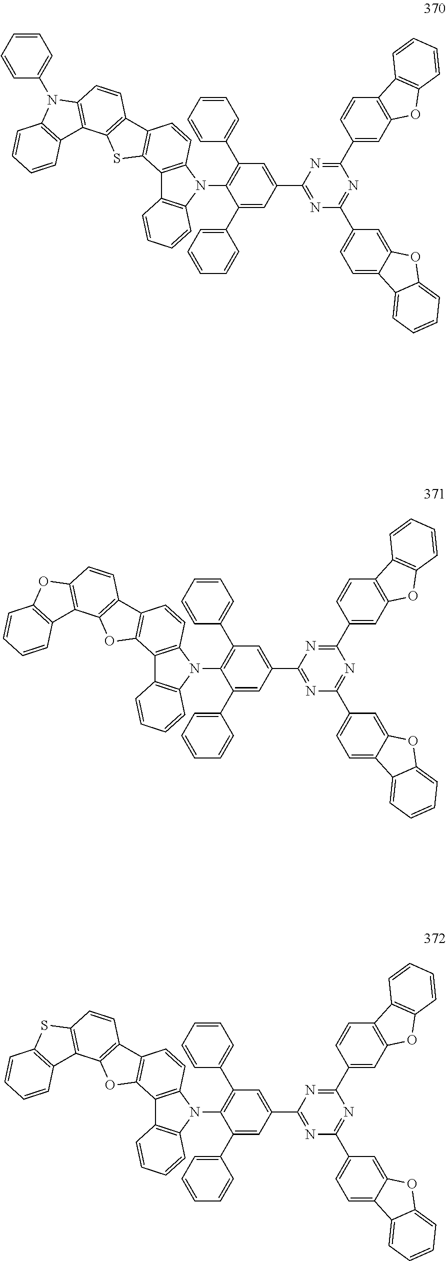 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4sc5c(ccc6c5c5ccccc5n6-c5ccccc5)c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5c(ccc6oc7ccccc7c65)c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5c(ccc6sc7ccccc7c65)c4ccc32)cc1]

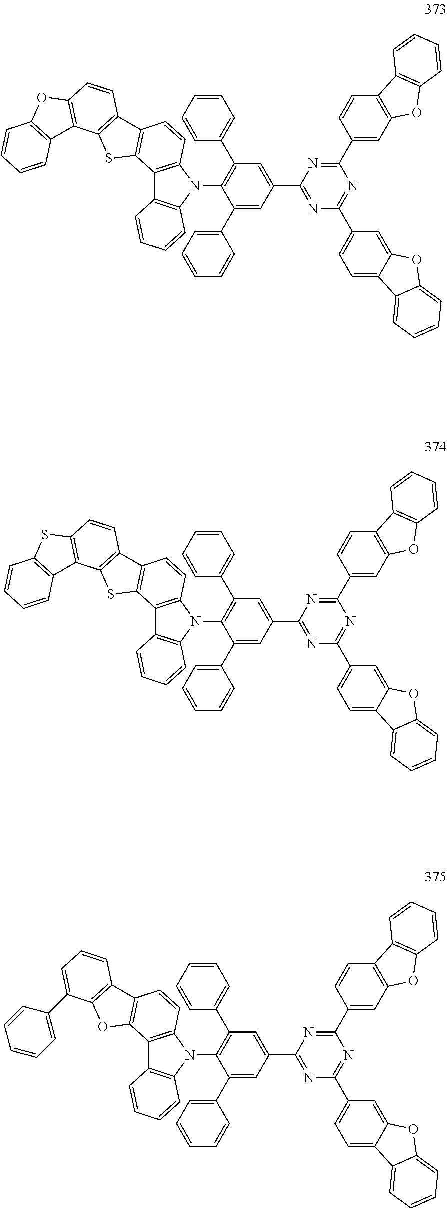 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4sc5c(ccc6oc7ccccc7c65)c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4sc5c(ccc6sc7ccccc7c65)c4ccc32)cc1 ; c1ccc(-c2cc(-c3nc(-c4ccc5c(c4)oc4ccccc45)nc(-c4ccc5c(c4)oc4ccccc45)n3)cc(-c3ccccc3)c2-n2c3ccccc3c3c4oc5c(-c6ccccc6)cccc5c4ccc32)cc1]

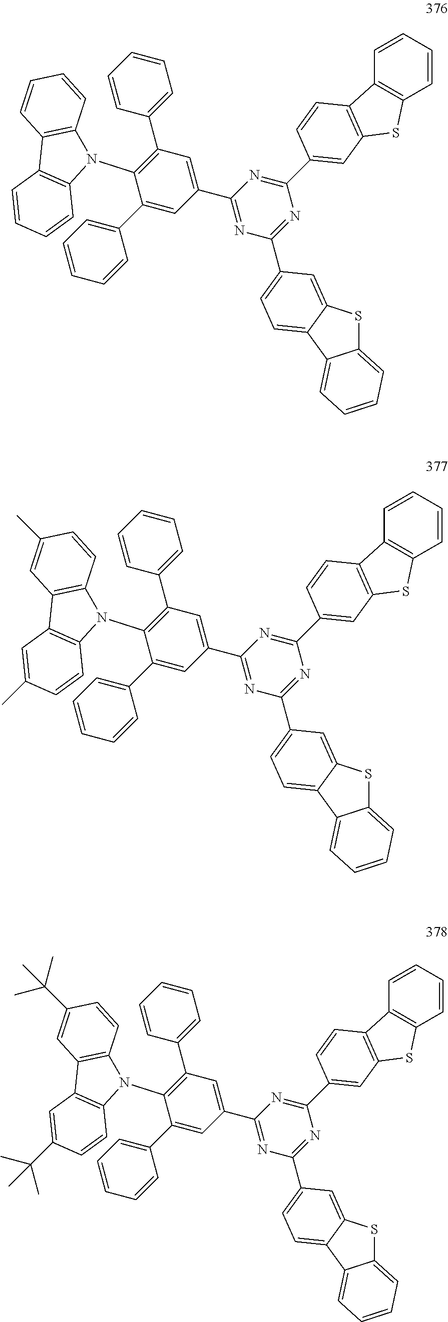 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccccc32)cc1 ; Cc1ccc2c(c1)c1cc(C)ccc1n2-c1c(-c2ccccc2)cc(-c2nc(-c3ccc4c(c3)sc3ccccc34)nc(-c3ccc4c(c3)sc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3ccc4c(c3)sc3ccccc34)nc(-c3ccc4c(c3)sc3ccccc34)n2)cc1-c1ccccc1]

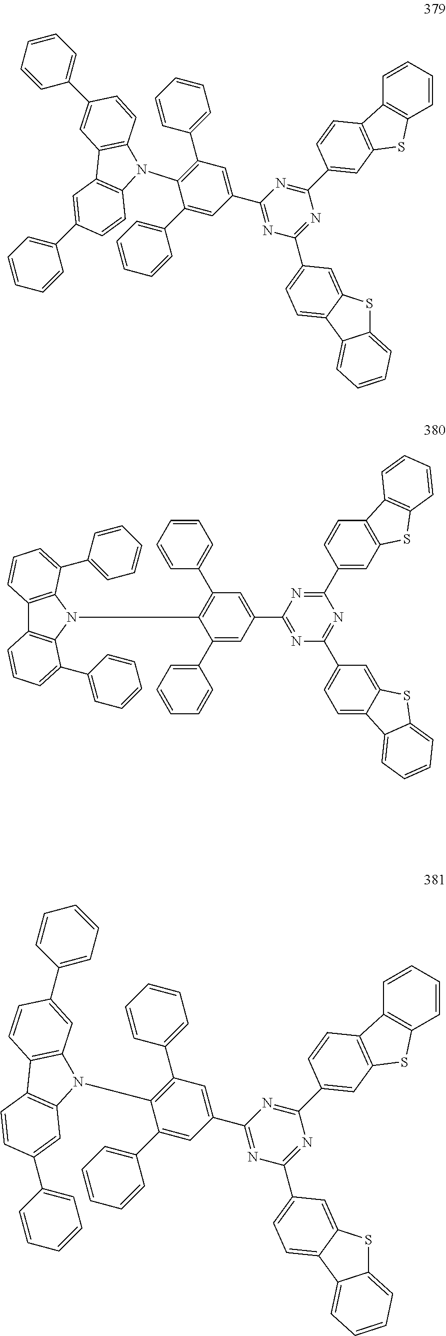 [SMILES: c1ccc(-c2ccc3c(c2)c2cc(-c4ccccc4)ccc2n3-c2c(-c3ccccc3)cc(-c3nc(-c4ccc5c(c4)sc4ccccc45)nc(-c4ccc5c(c4)sc4ccccc45)n3)cc2-c2ccccc2)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3c(-c4ccccc4)cccc3c3cccc(-c4ccccc4)c32)cc1 ; c1ccc(-c2ccc3c4ccc(-c5ccccc5)cc4n(-c4c(-c5ccccc5)cc(-c5nc(-c6ccc7c(c6)sc6ccccc67)nc(-c6ccc7c(c6)sc6ccccc67)n5)cc4-c4ccccc4)c3c2)cc1]

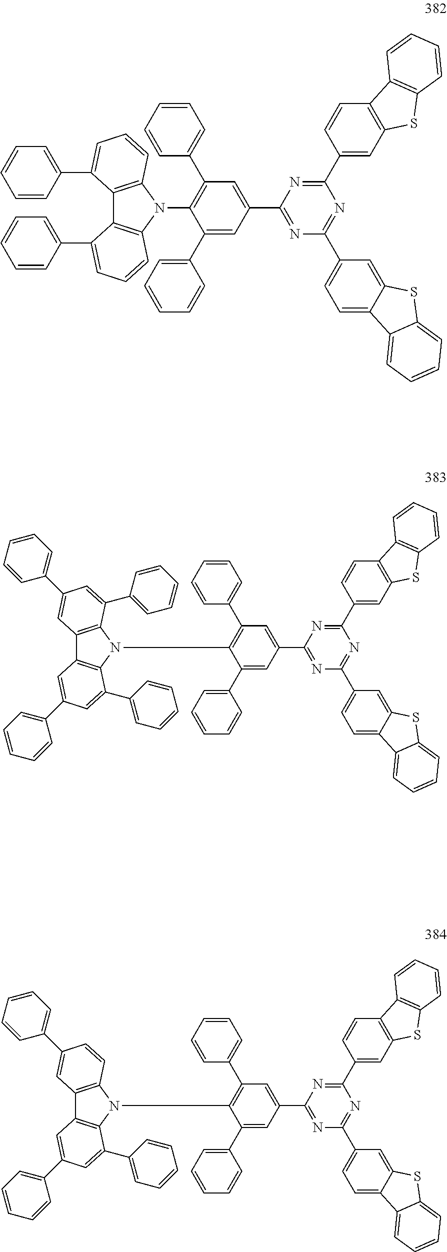 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3cccc(-c4ccccc4)c3c3c(-c4ccccc4)cccc32)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccc5c(c4)sc4ccccc45)nc(-c4ccc5c(c4)sc4ccccc45)n3)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccc5c(c4)sc4ccccc45)nc(-c4ccc5c(c4)sc4ccccc45)n3)cc2-c2ccccc2)cc1]

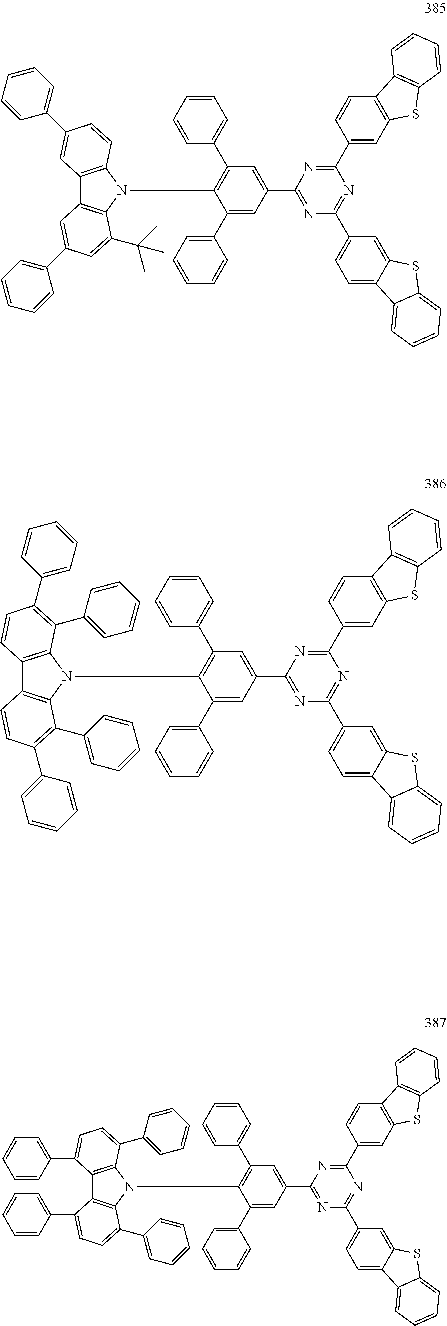 [SMILES: CC(C)(C)c1cc(-c2ccccc2)cc2c3cc(-c4ccccc4)ccc3n(-c3c(-c4ccccc4)cc(-c4nc(-c5ccc6c(c5)sc5ccccc56)nc(-c5ccc6c(c5)sc5ccccc56)n4)cc3-c3ccccc3)c12 ; c1ccc(-c2cc(-c3nc(-c4ccc5c(c4)sc4ccccc45)nc(-c4ccc5c(c4)sc4ccccc45)n3)cc(-c3ccccc3)c2-n2c3c(-c4ccccc4)c(-c4ccccc4)ccc3c3ccc(-c4ccccc4)c(-c4ccccc4)c32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3c(-c4ccccc4)ccc(-c4ccccc4)c3c3c(-c4ccccc4)ccc(-c4ccccc4)c32)cc1]

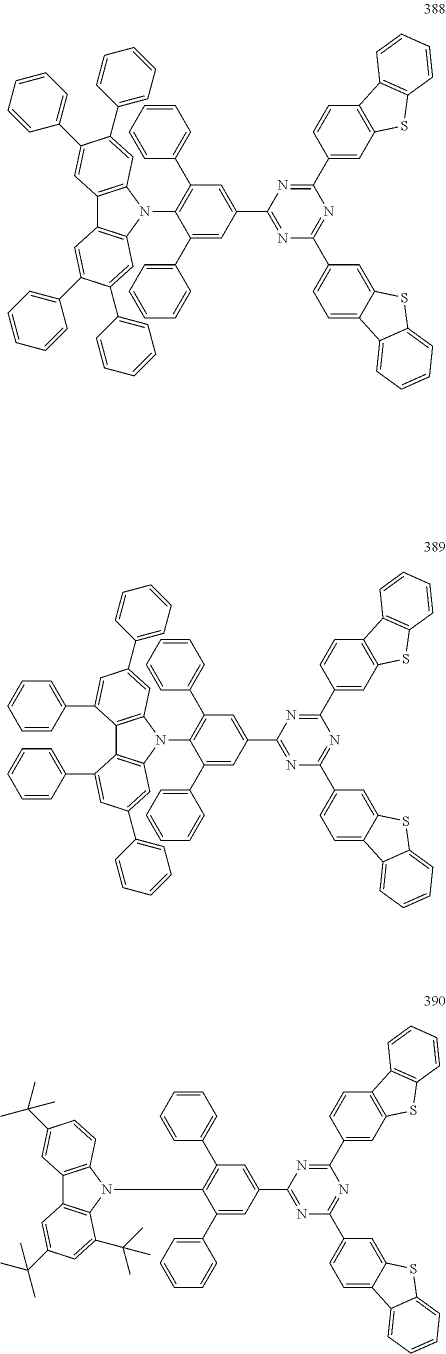 [SMILES: c1ccc(-c2cc3c4cc(-c5ccccc5)c(-c5ccccc5)cc4n(-c4c(-c5ccccc5)cc(-c5nc(-c6ccc7c(c6)sc6ccccc67)nc(-c6ccc7c(c6)sc6ccccc67)n5)cc4-c4ccccc4)c3cc2-c2ccccc2)cc1 ; c1ccc(-c2cc(-c3ccccc3)c3c4c(-c5ccccc5)cc(-c5ccccc5)cc4n(-c4c(-c5ccccc5)cc(-c5nc(-c6ccc7c(c6)sc6ccccc67)nc(-c6ccc7c(c6)sc6ccccc67)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccc4c(c3)sc3ccccc34)nc(-c3ccc4c(c3)sc3ccccc34)n2)cc1-c1ccccc1]

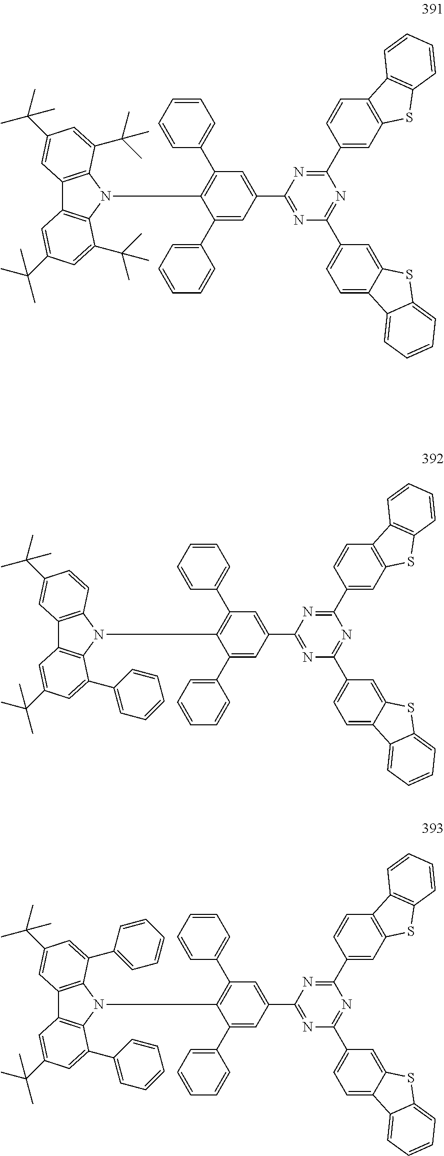 [SMILES: CC(C)(C)c1cc(C(C)(C)C)c2c(c1)c1cc(C(C)(C)C)cc(C(C)(C)C)c1n2-c1c(-c2ccccc2)cc(-c2nc(-c3ccc4c(c3)sc3ccccc34)nc(-c3ccc4c(c3)sc3ccccc34)n2)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccc4c(c3)sc3ccccc34)nc(-c3ccc4c(c3)sc3ccccc34)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccc4c(c3)sc3ccccc34)nc(-c3ccc4c(c3)sc3ccccc34)n2)cc1-c1ccccc1]

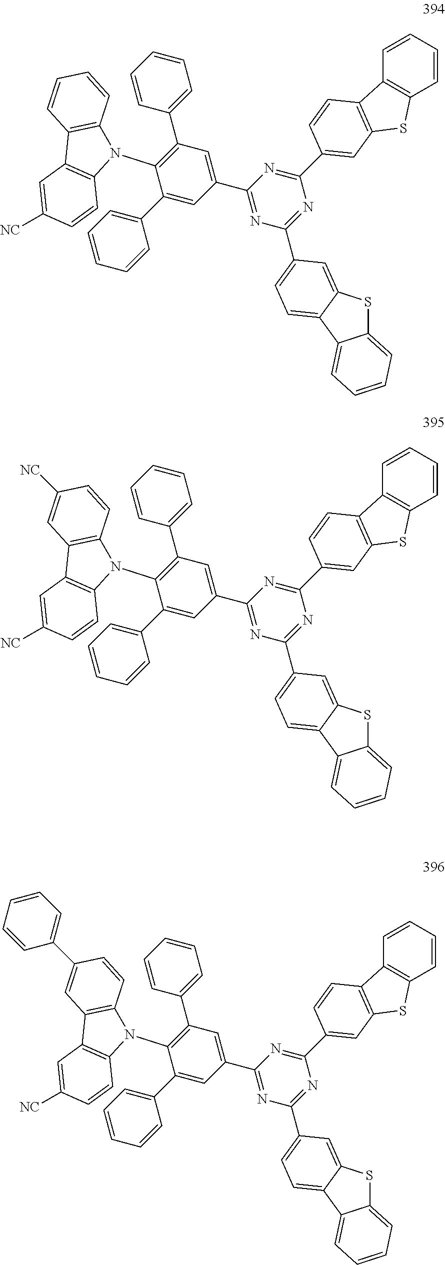 [SMILES: N#Cc1ccc2c(c1)c1ccccc1n2-c1c(-c2ccccc2)cc(-c2nc(-c3ccc4c(c3)sc3ccccc34)nc(-c3ccc4c(c3)sc3ccccc34)n2)cc1-c1ccccc1 ; N#Cc1ccc2c(c1)c1cc(C#N)ccc1n2-c1c(-c2ccccc2)cc(-c2nc(-c3ccc4c(c3)sc3ccccc34)nc(-c3ccc4c(c3)sc3ccccc34)n2)cc1-c1ccccc1 ; N#Cc1ccc2c(c1)c1cc(-c3ccccc3)ccc1n2-c1c(-c2ccccc2)cc(-c2nc(-c3ccc4c(c3)sc3ccccc34)nc(-c3ccc4c(c3)sc3ccccc34)n2)cc1-c1ccccc1]

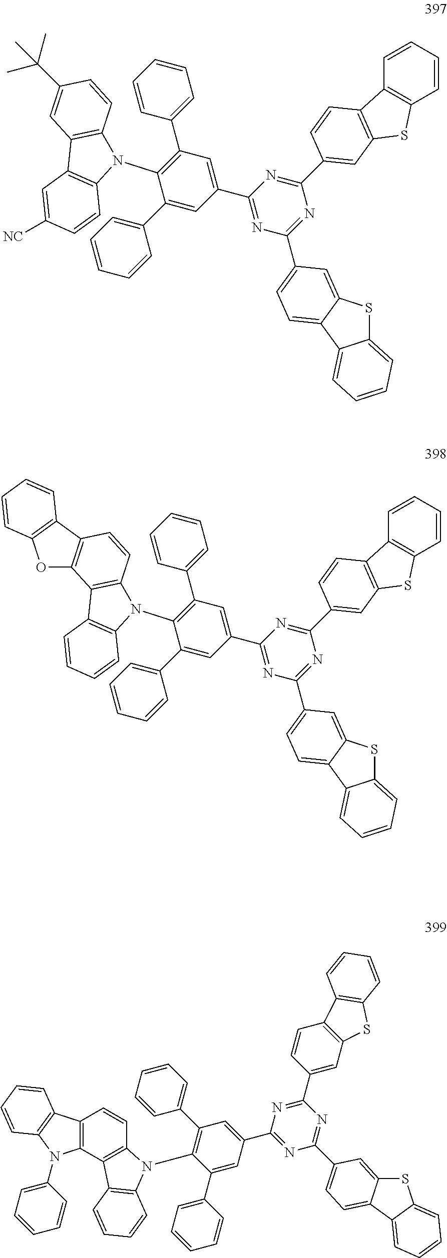 [SMILES: CC(C)(C)c1ccc2c(c1)c1cc(C#N)ccc1n2-c1c(-c2ccccc2)cc(-c2nc(-c3ccc4c(c3)sc3ccccc34)nc(-c3ccc4c(c3)sc3ccccc34)n2)cc1-c1ccccc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5ccccc5c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c2ccc2c4ccccc4n(-c4ccccc4)c23)cc1]

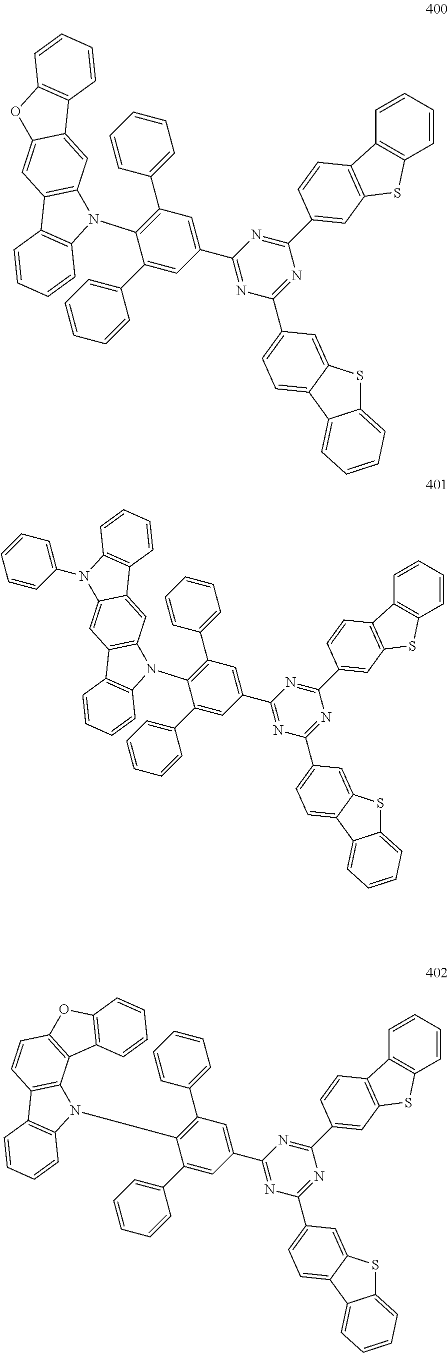 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3cc4oc5ccccc5c4cc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3cc4c(cc32)c2ccccc2n4-c2ccccc2)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccc4oc5ccccc5c4c32)cc1]

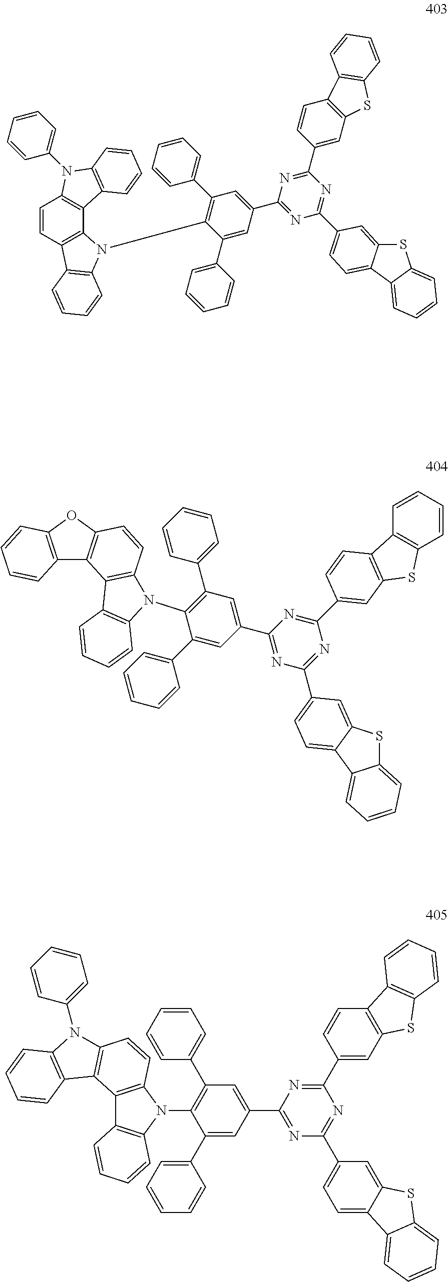 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccc4c(c5ccccc5n4-c4ccccc4)c32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4c(ccc32)oc2ccccc24)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4c5ccccc5n(-c5ccccc5)c4ccc32)cc1]

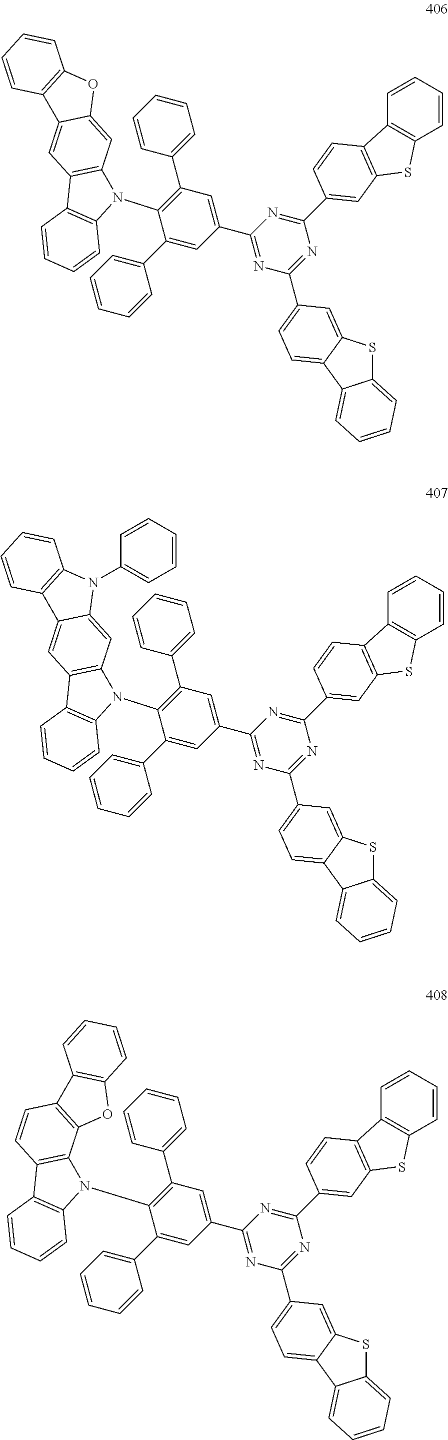 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3cc4c(cc32)oc2ccccc24)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3cc4c5ccccc5n(-c5ccccc5)c4cc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccc4c5ccccc5oc4c32)cc1]

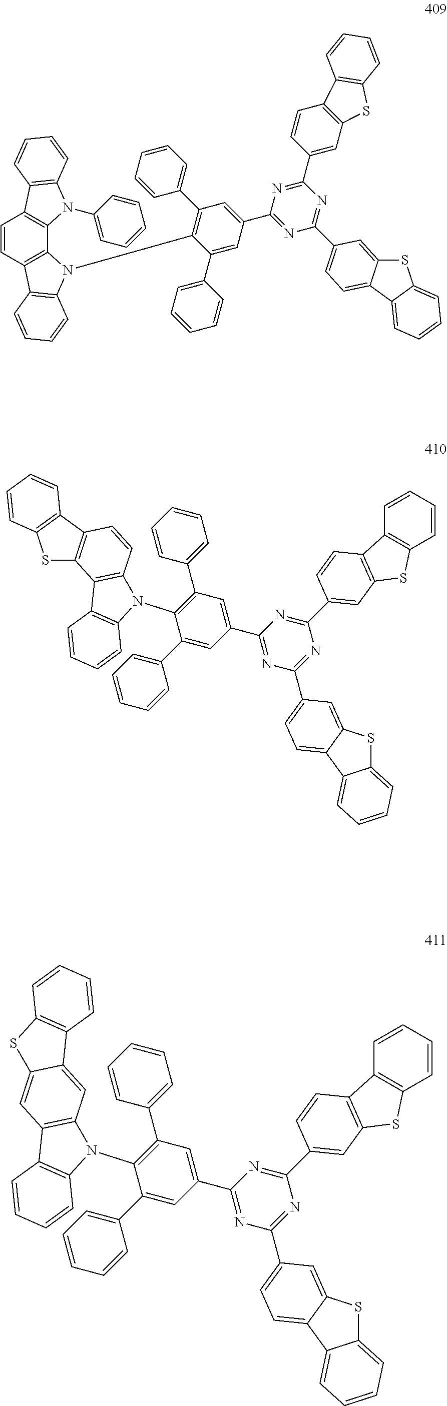 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccc4c5ccccc5n(-c5ccccc5)c4c32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4sc5ccccc5c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3cc4sc5ccccc5c4cc32)cc1]

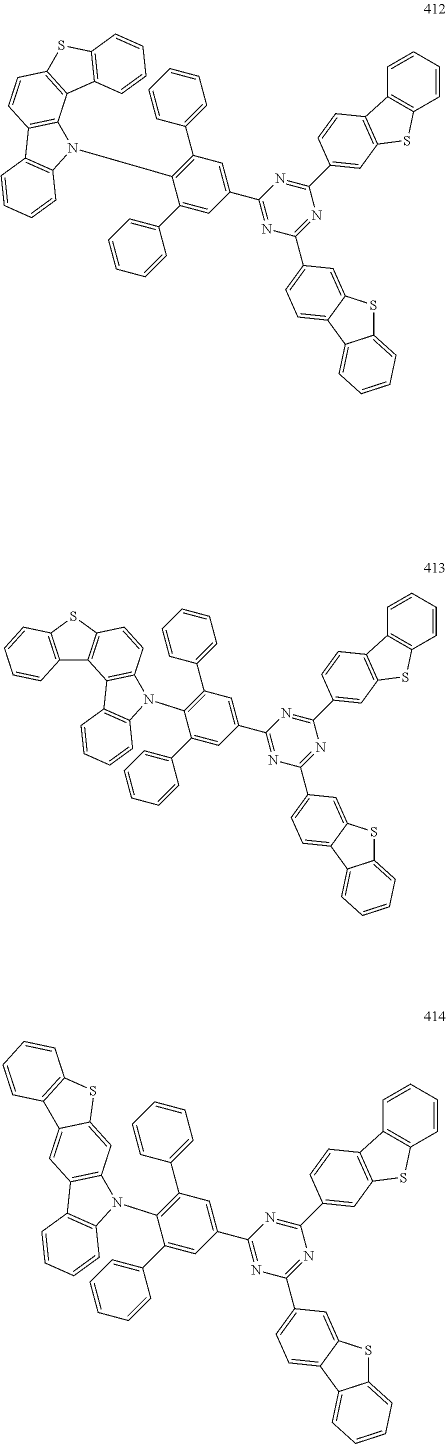 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccc4sc5ccccc5c4c32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4c(ccc32)sc2ccccc24)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3cc4c(cc32)sc2ccccc24)cc1]

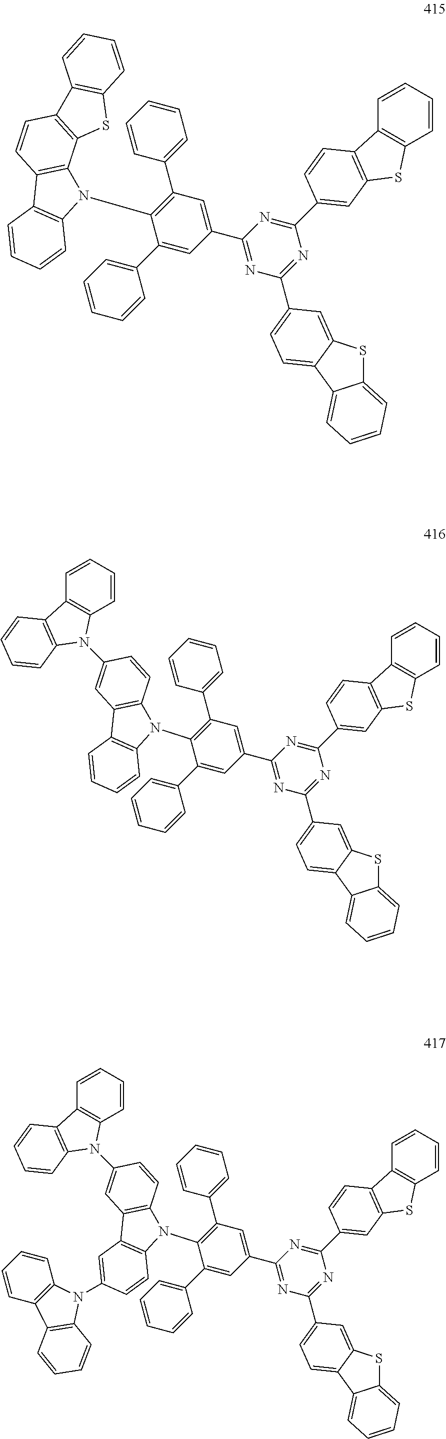 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccc4c5ccccc5sc4c32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3cc(-n4c5ccccc5c5ccccc54)ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccc(-n4c5ccccc5c5ccccc54)cc3c3cc(-n4c5ccccc5c5ccccc54)ccc32)cc1]

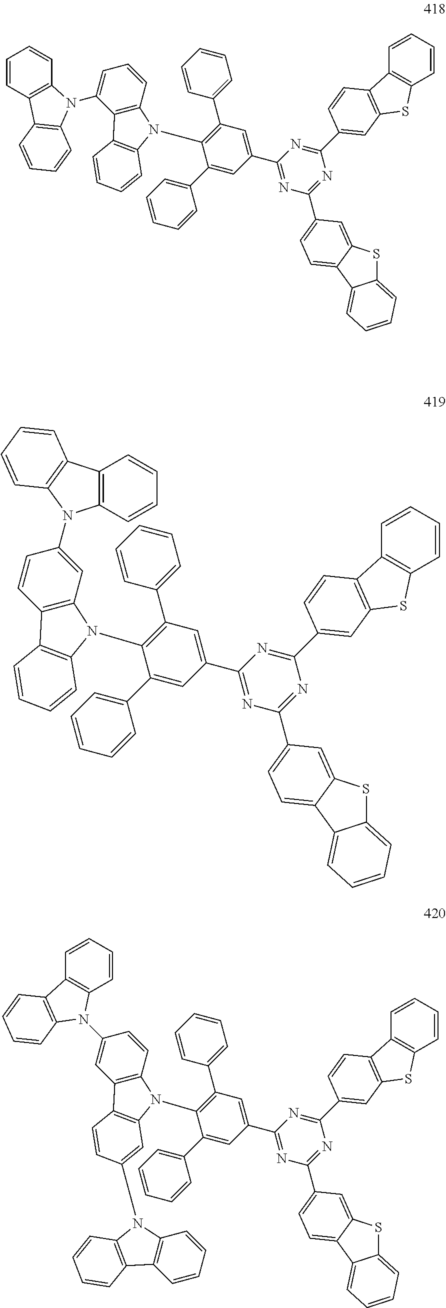 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c(-n4c5ccccc5c5ccccc54)cccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccc(-n4c5ccccc5c5ccccc54)cc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccc(-n4c5ccccc5c5ccccc54)cc3c3ccc(-n4c5ccccc5c5ccccc54)cc32)cc1]

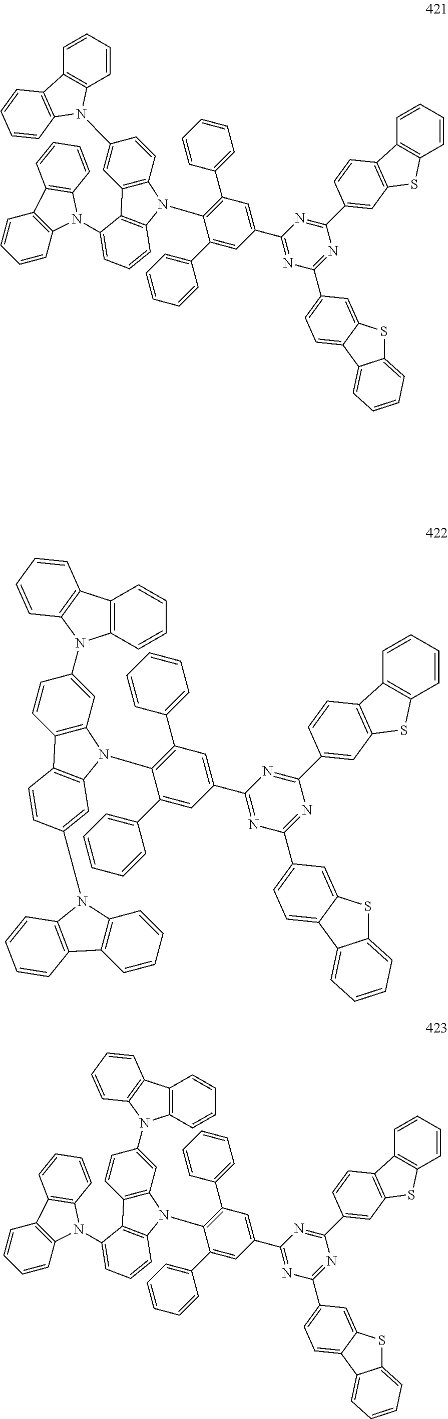 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccc(-n4c5ccccc5c5ccccc54)cc3c3c(-n4c5ccccc5c5ccccc54)cccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3cc(-n4c5ccccc5c5ccccc54)ccc3c3ccc(-n4c5ccccc5c5ccccc54)cc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3cc(-n4c5ccccc5c5ccccc54)ccc3c3c(-n4c5ccccc5c5ccccc54)cccc32)cc1]

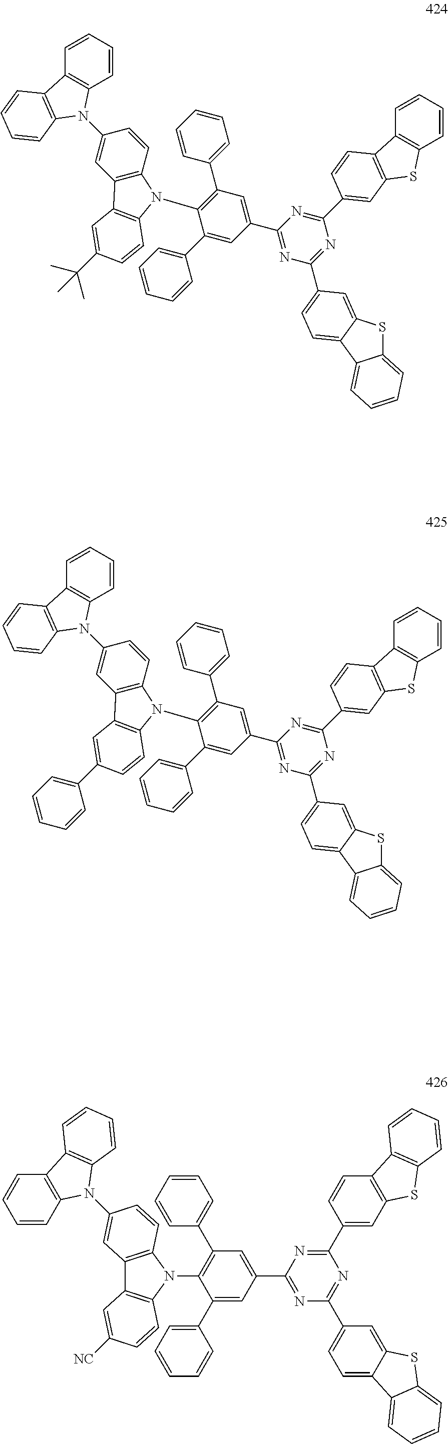 [SMILES: CC(C)(C)c1ccc2c(c1)c1cc(-n3c4ccccc4c4ccccc43)ccc1n2-c1c(-c2ccccc2)cc(-c2nc(-c3ccc4c(c3)sc3ccccc34)nc(-c3ccc4c(c3)sc3ccccc34)n2)cc1-c1ccccc1 ; c1ccc(-c2ccc3c(c2)c2cc(-n4c5ccccc5c5ccccc54)ccc2n3-c2c(-c3ccccc3)cc(-c3nc(-c4ccc5c(c4)sc4ccccc45)nc(-c4ccc5c(c4)sc4ccccc45)n3)cc2-c2ccccc2)cc1 ; N#Cc1ccc2c(c1)c1cc(-n3c4ccccc4c4ccccc43)ccc1n2-c1c(-c2ccccc2)cc(-c2nc(-c3ccc4c(c3)sc3ccccc34)nc(-c3ccc4c(c3)sc3ccccc34)n2)cc1-c1ccccc1]

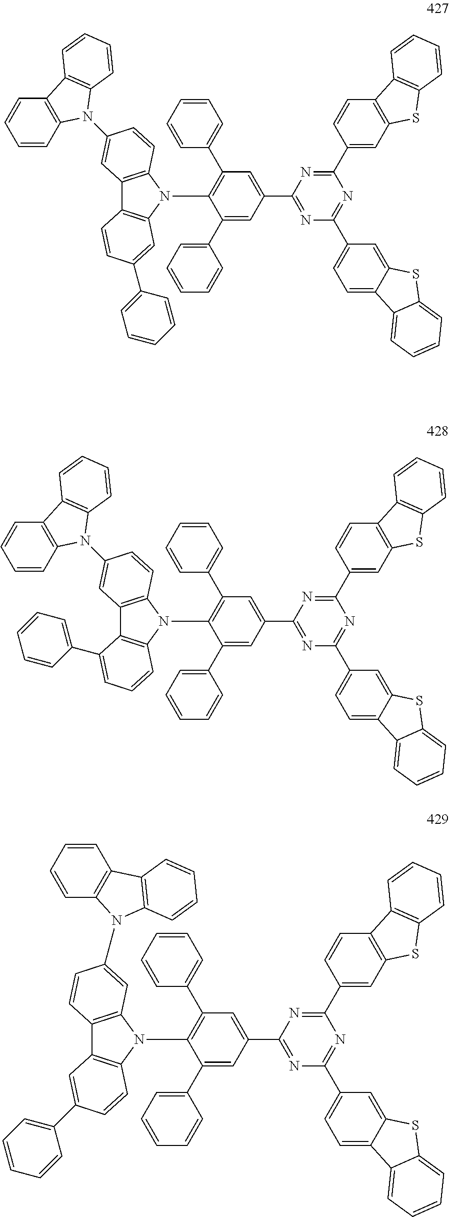 [SMILES: c1ccc(-c2ccc3c4cc(-n5c6ccccc6c6ccccc65)ccc4n(-c4c(-c5ccccc5)cc(-c5nc(-c6ccc7c(c6)sc6ccccc67)nc(-c6ccc7c(c6)sc6ccccc67)n5)cc4-c4ccccc4)c3c2)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccc(-n4c5ccccc5c5ccccc54)cc3c3c(-c4ccccc4)cccc32)cc1 ; c1ccc(-c2ccc3c(c2)c2ccc(-n4c5ccccc5c5ccccc54)cc2n3-c2c(-c3ccccc3)cc(-c3nc(-c4ccc5c(c4)sc4ccccc45)nc(-c4ccc5c(c4)sc4ccccc45)n3)cc2-c2ccccc2)cc1]

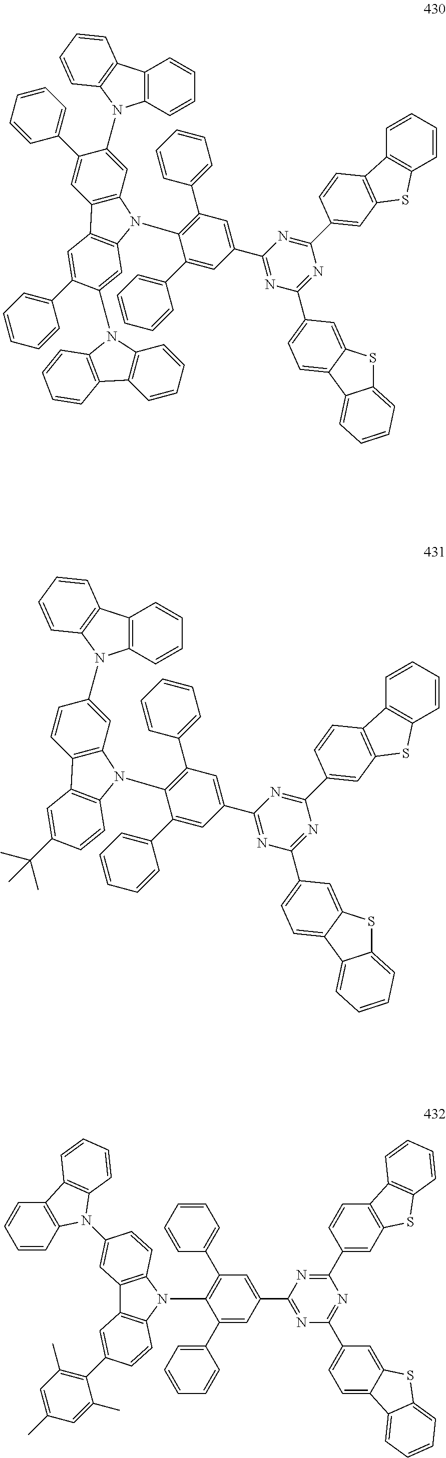 [SMILES: c1ccc(-c2cc3c4cc(-c5ccccc5)c(-n5c6ccccc6c6ccccc65)cc4n(-c4c(-c5ccccc5)cc(-c5nc(-c6ccc7c(c6)sc6ccccc67)nc(-c6ccc7c(c6)sc6ccccc67)n5)cc4-c4ccccc4)c3cc2-n2c3ccccc3c3ccccc32)cc1 ; CC(C)(C)c1ccc2c(c1)c1ccc(-n3c4ccccc4c4ccccc43)cc1n2-c1c(-c2ccccc2)cc(-c2nc(-c3ccc4c(c3)sc3ccccc34)nc(-c3ccc4c(c3)sc3ccccc34)n2)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccc5c(c4)sc4ccccc45)nc(-c4ccc5c(c4)sc4ccccc45)n3)cc2-c2ccccc2)c(C)c1]

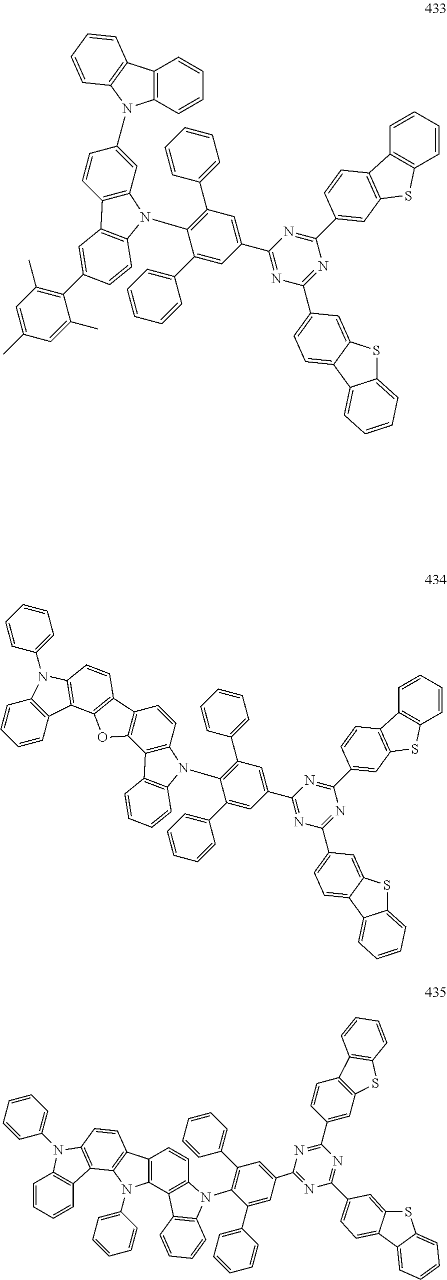 [SMILES: Cc1cc(C)c(-c2ccc3c(c2)c2ccc(-n4c5ccccc5c5ccccc54)cc2n3-c2c(-c3ccccc3)cc(-c3nc(-c4ccc5c(c4)sc4ccccc45)nc(-c4ccc5c(c4)sc4ccccc45)n3)cc2-c2ccccc2)c(C)c1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6c5c5ccccc5n6-c5ccccc5)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c2ccc2c4ccc5c(c6ccccc6n5-c5ccccc5)c4n(-c4ccccc4)c23)cc1]

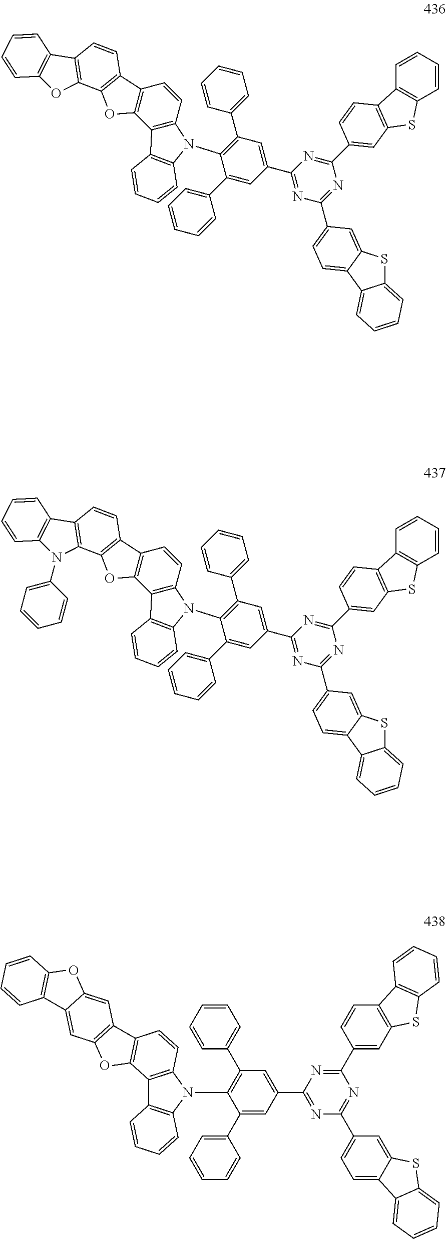 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6c7ccccc7oc65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6c7ccccc7n(-c7ccccc7)c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6c(cc5c4ccc32)oc2ccccc26)cc1]

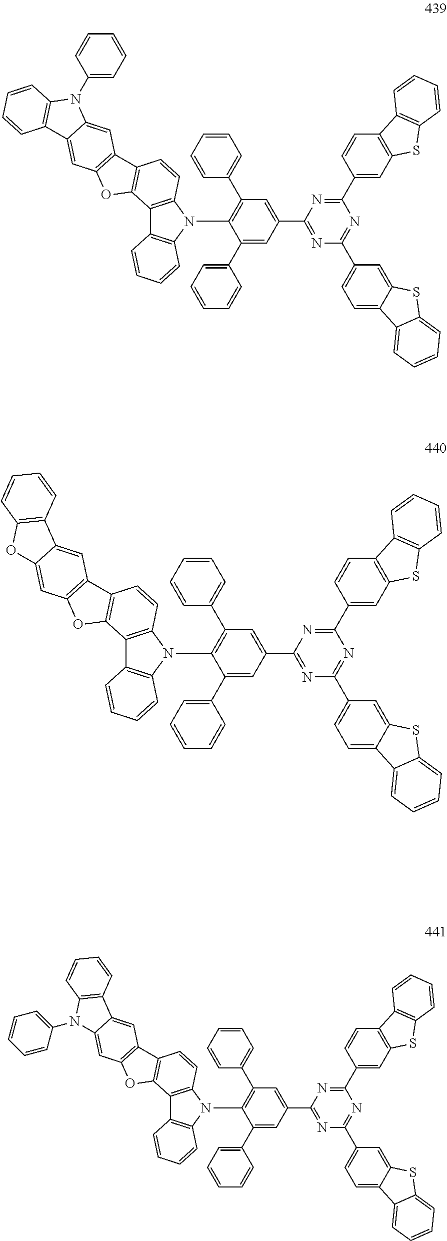 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6c7ccccc7n(-c7ccccc7)c6cc5c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6oc7ccccc7c6cc5c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6c(cc5c4ccc32)c2ccccc2n6-c2ccccc2)cc1]

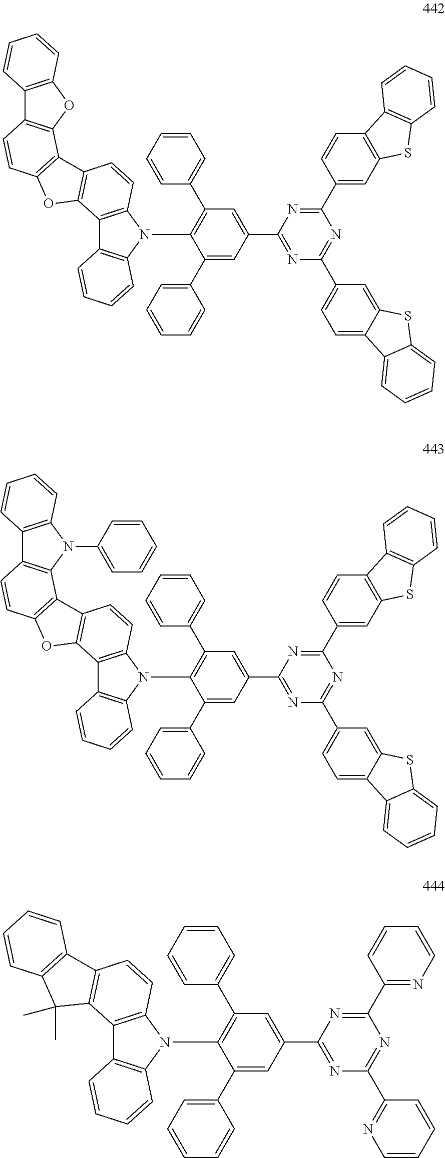 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5ccc6c7ccccc7oc6c5c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5ccc6c7ccccc7n(-c7ccccc7)c6c5c4ccc32)cc1 ; CC1(C)c2ccccc2-c2ccc3c(c21)c1ccccc1n3-c1c(-c2ccccc2)cc(-c2nc(-c3ccccn3)nc(-c3ccccn3)n2)cc1-c1ccccc1]

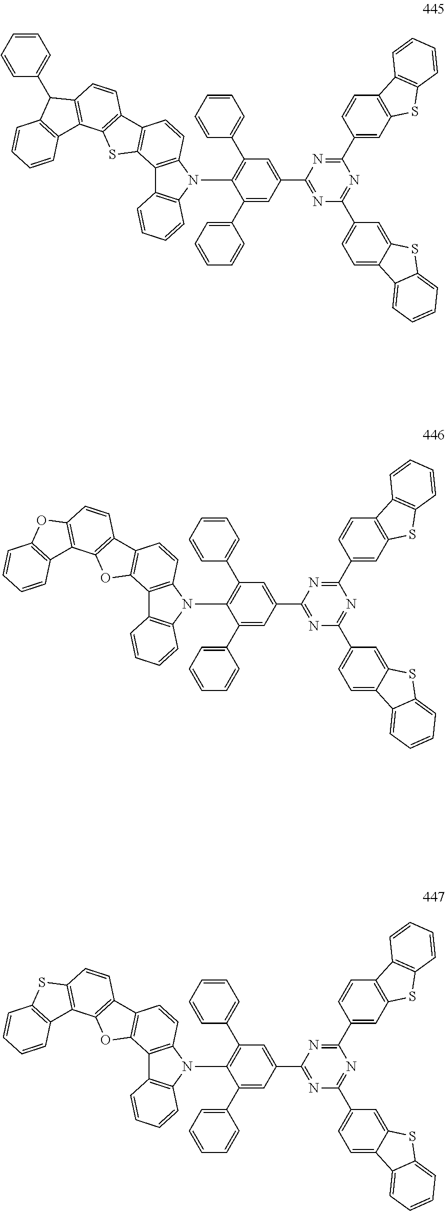 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4sc5c6c(ccc5c4ccc32)C(c2ccccc2)c2ccccc2-6)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6oc7ccccc7c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6sc7ccccc7c65)c4ccc32)cc1]

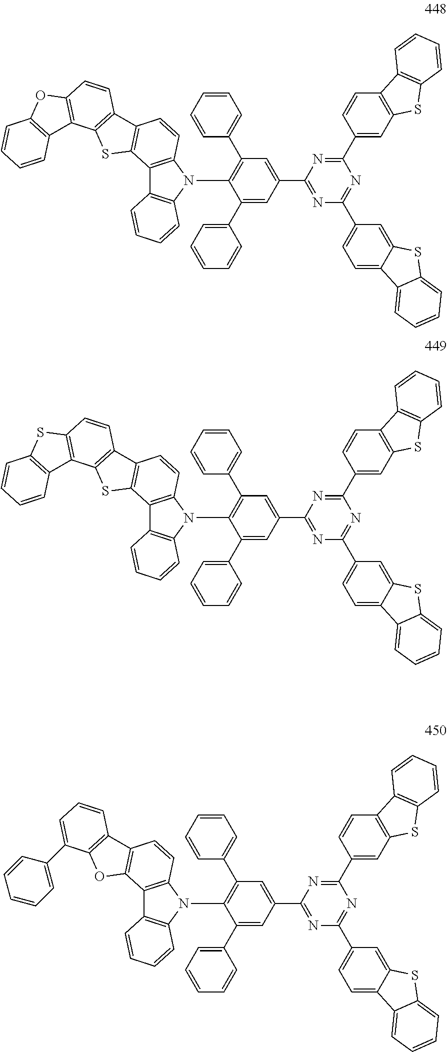 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4sc5c(ccc6oc7ccccc7c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4sc5c(ccc6sc7ccccc7c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(-c6ccccc6)cccc5c4ccc32)cc1]

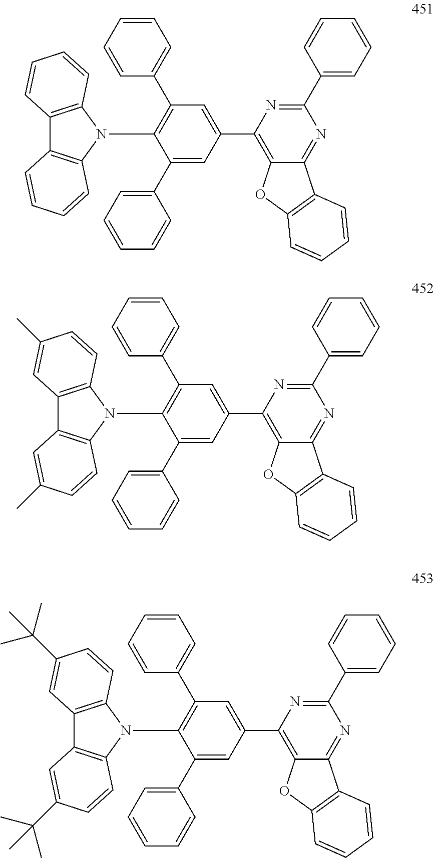

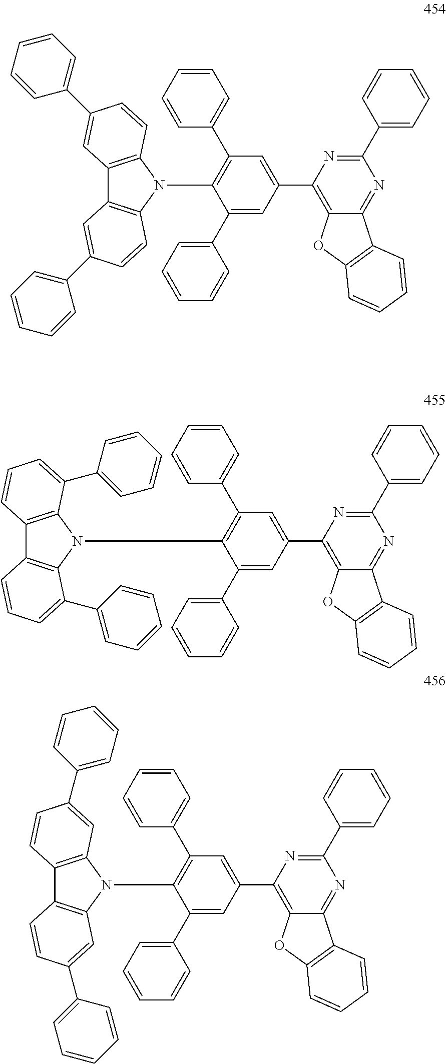 [SMILES: c1ccc(-c2ccc3c(c2)c2cc(-c4ccccc4)ccc2n3-c2c(-c3ccccc3)cc(-c3nc(-c4ccccc4)nc4c3oc3ccccc34)cc2-c2ccccc2)cc1 ; c1ccc(-c2nc(-c3cc(-c4ccccc4)c(-n4c5c(-c6ccccc6)cccc5c5cccc(-c6ccccc6)c54)c(-c4ccccc4)c3)c3oc4ccccc4c3n2)cc1 ; c1ccc(-c2ccc3c4ccc(-c5ccccc5)cc4n(-c4c(-c5ccccc5)cc(-c5nc(-c6ccccc6)nc6c5oc5ccccc56)cc4-c4ccccc4)c3c2)cc1]

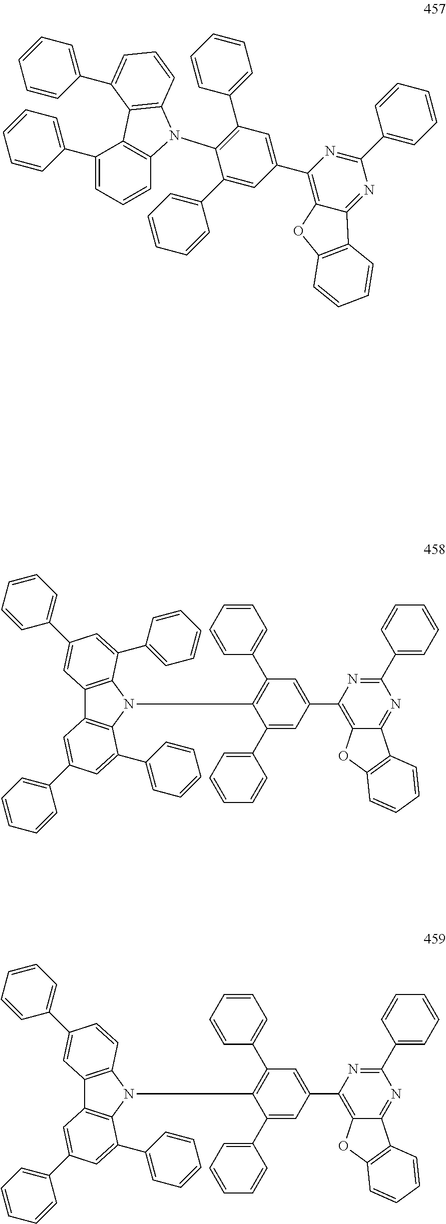 [SMILES: c1ccc(-c2nc(-c3cc(-c4ccccc4)c(-n4c5cccc(-c6ccccc6)c5c5c(-c6ccccc6)cccc54)c(-c4ccccc4)c3)c3oc4ccccc4c3n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)nc4c3oc3ccccc34)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccccc4)nc4c3oc3ccccc34)cc2-c2ccccc2)cc1]

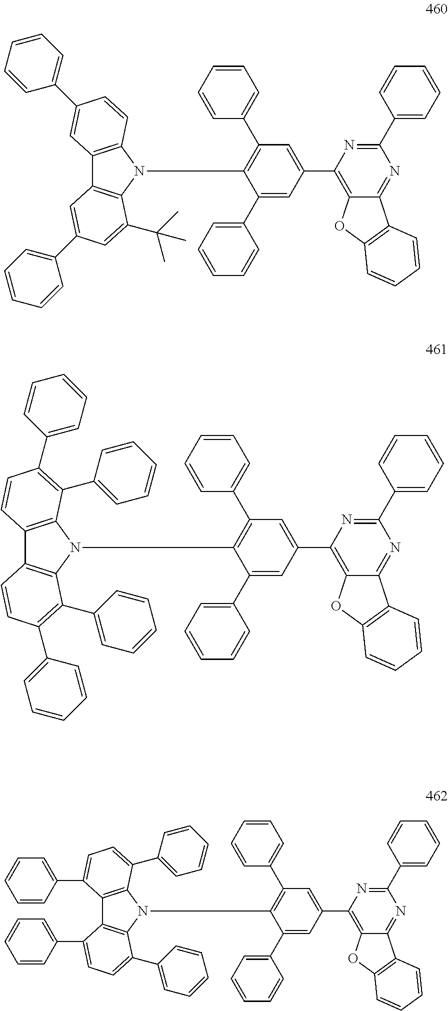 [SMILES: CC(C)(C)c1cc(-c2ccccc2)cc2c3cc(-c4ccccc4)ccc3n(-c3c(-c4ccccc4)cc(-c4nc(-c5ccccc5)nc5c4oc4ccccc45)cc3-c3ccccc3)c12 ; c1ccc(-c2nc(-c3cc(-c4ccccc4)c(-n4c5c(-c6ccccc6)c(-c6ccccc6)ccc5c5ccc(-c6ccccc6)c(-c6ccccc6)c54)c(-c4ccccc4)c3)c3oc4ccccc4c3n2)cc1 ; c1ccc(-c2nc(-c3cc(-c4ccccc4)c(-n4c5c(-c6ccccc6)ccc(-c6ccccc6)c5c5c(-c6ccccc6)ccc(-c6ccccc6)c54)c(-c4ccccc4)c3)c3oc4ccccc4c3n2)cc1]

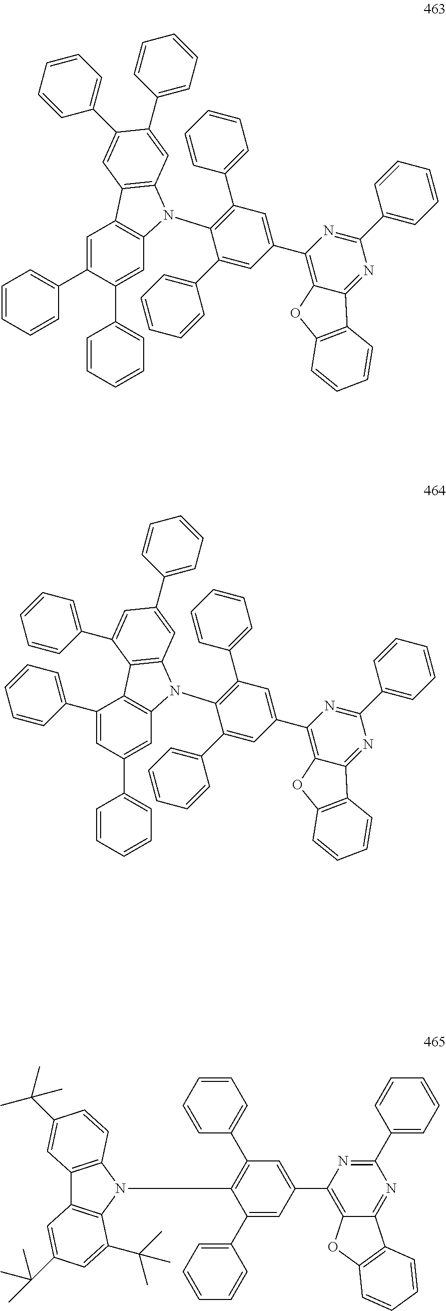 [SMILES: c1ccc(-c2nc(-c3cc(-c4ccccc4)c(-n4c5cc(-c6ccccc6)c(-c6ccccc6)cc5c5cc(-c6ccccc6)c(-c6ccccc6)cc54)c(-c4ccccc4)c3)c3oc4ccccc4c3n2)cc1 ; c1ccc(-c2cc(-c3ccccc3)c3c4c(-c5ccccc5)cc(-c5ccccc5)cc4n(-c4c(-c5ccccc5)cc(-c5nc(-c6ccccc6)nc6c5oc5ccccc56)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)nc3c2oc2ccccc23)cc1-c1ccccc1]

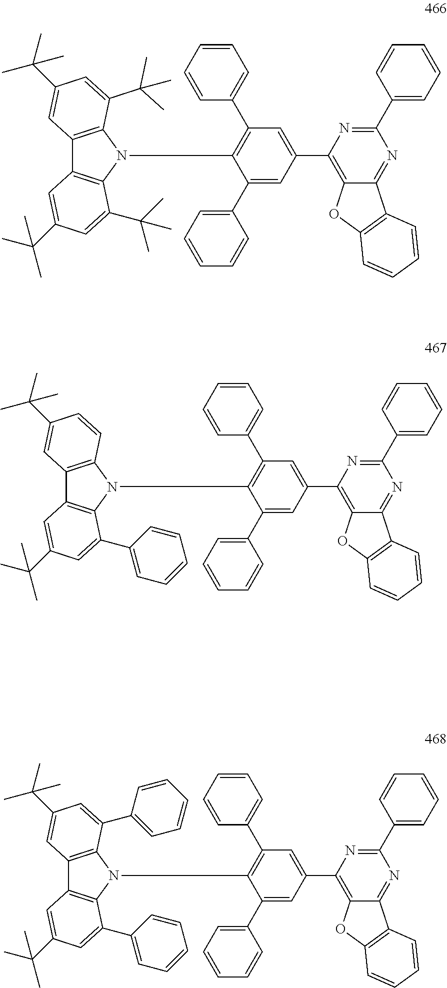 [SMILES: CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)nc3c2oc2ccccc23)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccccc3)nc3c2oc2ccccc23)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccccc3)nc3c2oc2ccccc23)cc1-c1ccccc1]

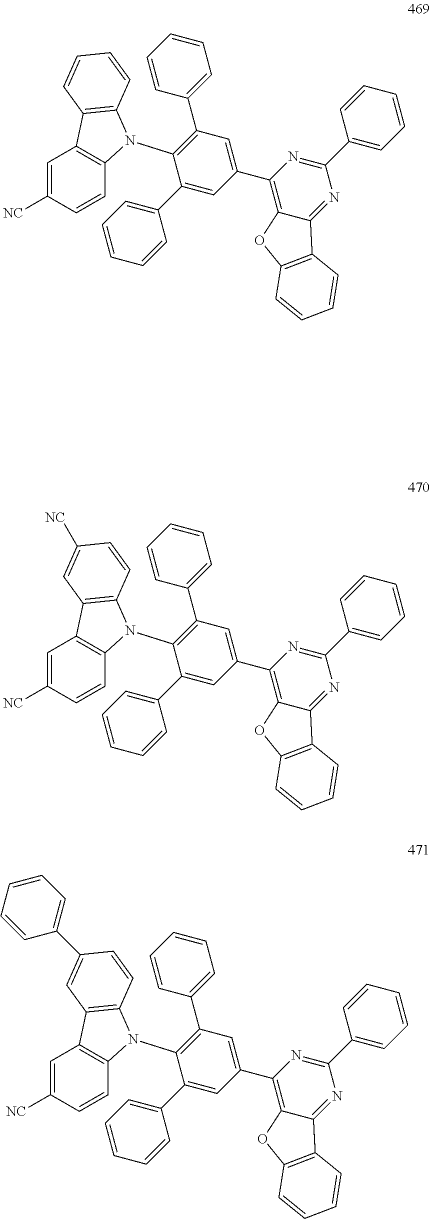 [SMILES: N#Cc1ccc2c(c1)c1ccccc1n2-c1c(-c2ccccc2)cc(-c2nc(-c3ccccc3)nc3c2oc2ccccc23)cc1-c1ccccc1 ; N#Cc1ccc2c(c1)c1cc(C#N)ccc1n2-c1c(-c2ccccc2)cc(-c2nc(-c3ccccc3)nc3c2oc2ccccc23)cc1-c1ccccc1 ; N#Cc1ccc2c(c1)c1cc(-c3ccccc3)ccc1n2-c1c(-c2ccccc2)cc(-c2nc(-c3ccccc3)nc3c2oc2ccccc23)cc1-c1ccccc1]

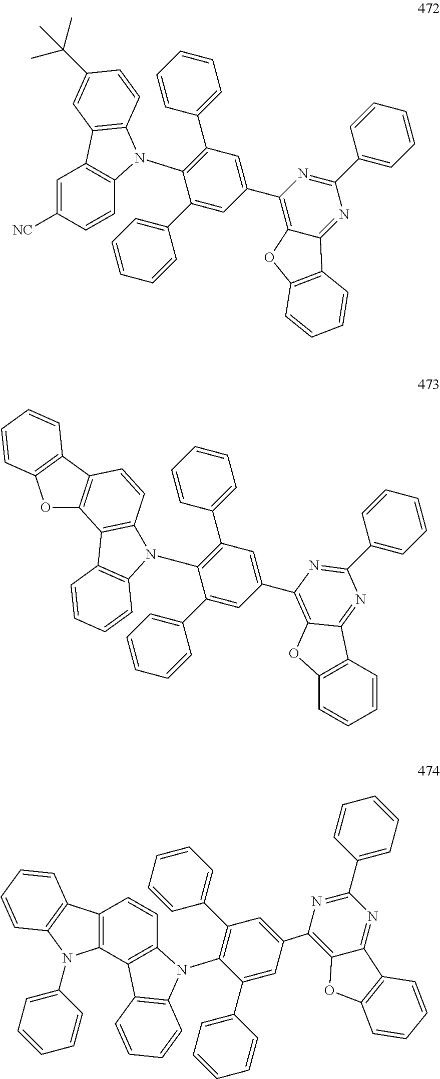 [SMILES: CC(C)(C)c1ccc2c(c1)c1cc(C#N)ccc1n2-c1c(-c2ccccc2)cc(-c2nc(-c3ccccc3)nc3c2oc2ccccc23)cc1-c1ccccc1 ; c1ccc(-c2nc(-c3cc(-c4ccccc4)c(-n4c5ccccc5c5c6oc7ccccc7c6ccc54)c(-c4ccccc4)c3)c3oc4ccccc4c3n2)cc1 ; c1ccc(-c2nc(-c3cc(-c4ccccc4)c(-n4c5ccccc5c5c4ccc4c6ccccc6n(-c6ccccc6)c45)c(-c4ccccc4)c3)c3oc4ccccc4c3n2)cc1]

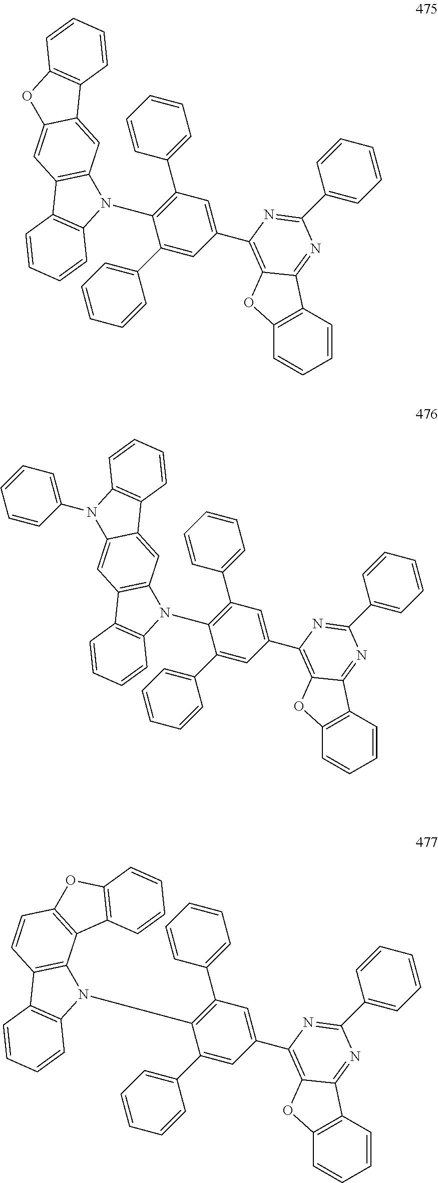 [SMILES: c1ccc(-c2nc(-c3cc(-c4ccccc4)c(-n4c5ccccc5c5cc6oc7ccccc7c6cc54)c(-c4ccccc4)c3)c3oc4ccccc4c3n2)cc1 ; c1ccc(-c2nc(-c3cc(-c4ccccc4)c(-n4c5ccccc5c5cc6c(cc54)c4ccccc4n6-c4ccccc4)c(-c4ccccc4)c3)c3oc4ccccc4c3n2)cc1 ; c1ccc(-c2nc(-c3cc(-c4ccccc4)c(-n4c5ccccc5c5ccc6oc7ccccc7c6c54)c(-c4ccccc4)c3)c3oc4ccccc4c3n2)cc1]

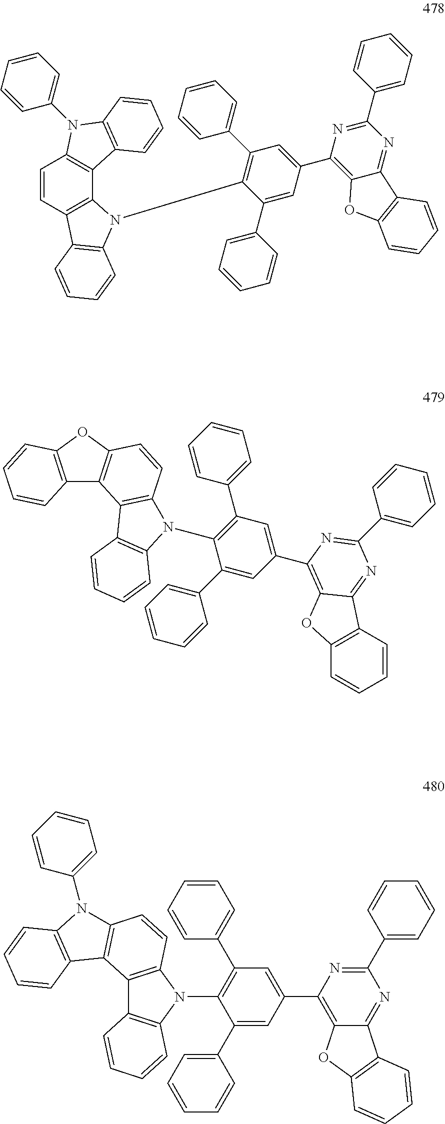 [SMILES: c1ccc(-c2nc(-c3cc(-c4ccccc4)c(-n4c5ccccc5c5ccc6c(c7ccccc7n6-c6ccccc6)c54)c(-c4ccccc4)c3)c3oc4ccccc4c3n2)cc1 ; c1ccc(-c2nc(-c3cc(-c4ccccc4)c(-n4c5ccccc5c5c6c(ccc54)oc4ccccc46)c(-c4ccccc4)c3)c3oc4ccccc4c3n2)cc1 ; c1ccc(-c2nc(-c3cc(-c4ccccc4)c(-n4c5ccccc5c5c6c7ccccc7n(-c7ccccc7)c6ccc54)c(-c4ccccc4)c3)c3oc4ccccc4c3n2)cc1]

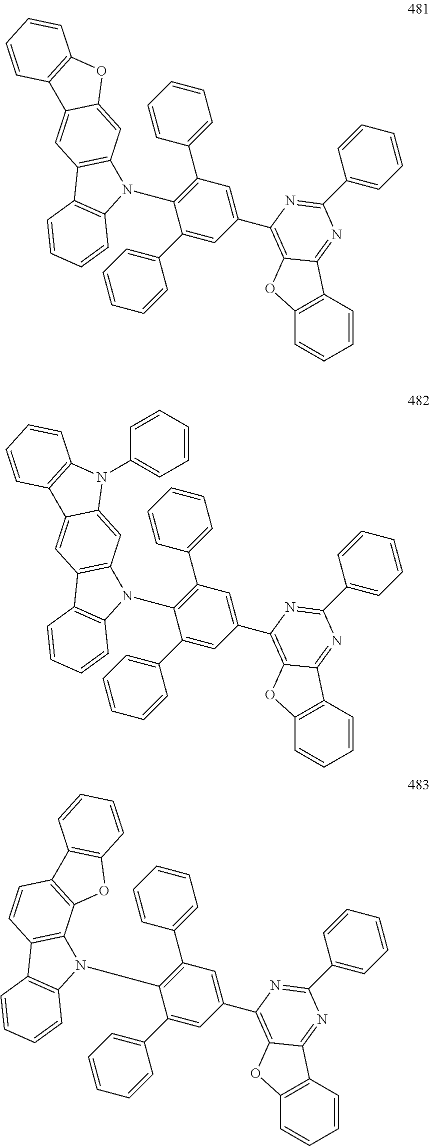 [SMILES: c1ccc(-c2nc(-c3cc(-c4ccccc4)c(-n4c5ccccc5c5cc6c(cc54)oc4ccccc46)c(-c4ccccc4)c3)c3oc4ccccc4c3n2)cc1 ; c1ccc(-c2nc(-c3cc(-c4ccccc4)c(-n4c5ccccc5c5cc6c7ccccc7n(-c7ccccc7)c6cc54)c(-c4ccccc4)c3)c3oc4ccccc4c3n2)cc1 ; c1ccc(-c2nc(-c3cc(-c4ccccc4)c(-n4c5ccccc5c5ccc6c7ccccc7oc6c54)c(-c4ccccc4)c3)c3oc4ccccc4c3n2)cc1]

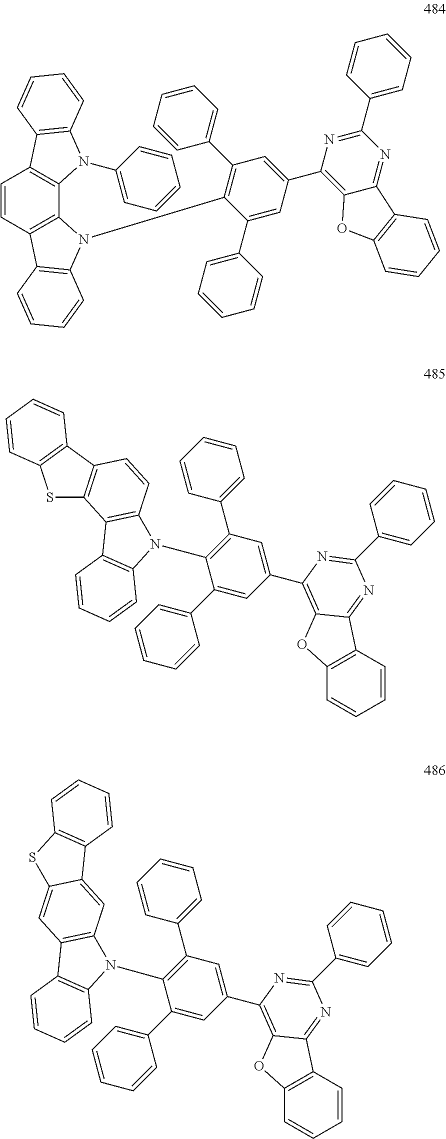 [SMILES: c1ccc(-c2nc(-c3cc(-c4ccccc4)c(-n4c5ccccc5c5ccc6c7ccccc7n(-c7ccccc7)c6c54)c(-c4ccccc4)c3)c3oc4ccccc4c3n2)cc1 ; c1ccc(-c2nc(-c3cc(-c4ccccc4)c(-n4c5ccccc5c5c6sc7ccccc7c6ccc54)c(-c4ccccc4)c3)c3oc4ccccc4c3n2)cc1 ; c1ccc(-c2nc(-c3cc(-c4ccccc4)c(-n4c5ccccc5c5cc6sc7ccccc7c6cc54)c(-c4ccccc4)c3)c3oc4ccccc4c3n2)cc1]

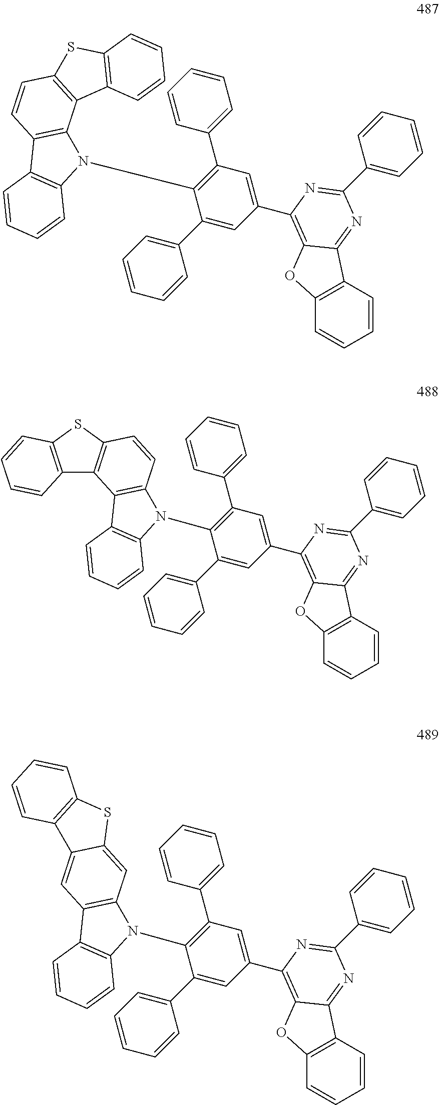 [SMILES: c1ccc(-c2nc(-c3cc(-c4ccccc4)c(-n4c5ccccc5c5ccc6sc7ccccc7c6c54)c(-c4ccccc4)c3)c3oc4ccccc4c3n2)cc1 ; c1ccc(-c2nc(-c3cc(-c4ccccc4)c(-n4c5ccccc5c5c6c(ccc54)sc4ccccc46)c(-c4ccccc4)c3)c3oc4ccccc4c3n2)cc1 ; c1ccc(-c2nc(-c3cc(-c4ccccc4)c(-n4c5ccccc5c5cc6c(cc54)sc4ccccc46)c(-c4ccccc4)c3)c3oc4ccccc4c3n2)cc1]

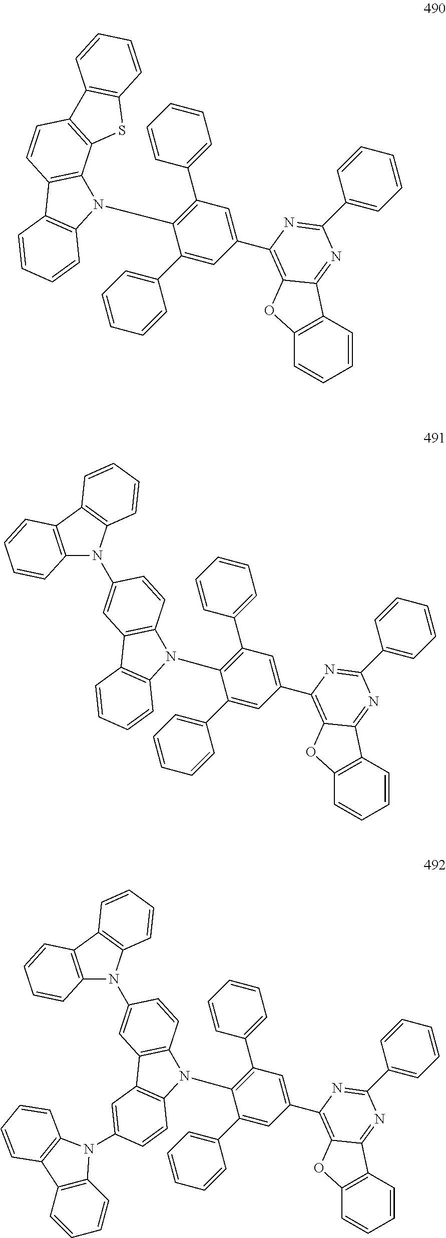 [SMILES: c1ccc(-c2nc(-c3cc(-c4ccccc4)c(-n4c5ccccc5c5ccc6c7ccccc7sc6c54)c(-c4ccccc4)c3)c3oc4ccccc4c3n2)cc1 ; c1ccc(-c2nc(-c3cc(-c4ccccc4)c(-n4c5ccccc5c5cc(-n6c7ccccc7c7ccccc76)ccc54)c(-c4ccccc4)c3)c3oc4ccccc4c3n2)cc1 ; c1ccc(-c2nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)c3oc4ccccc4c3n2)cc1]

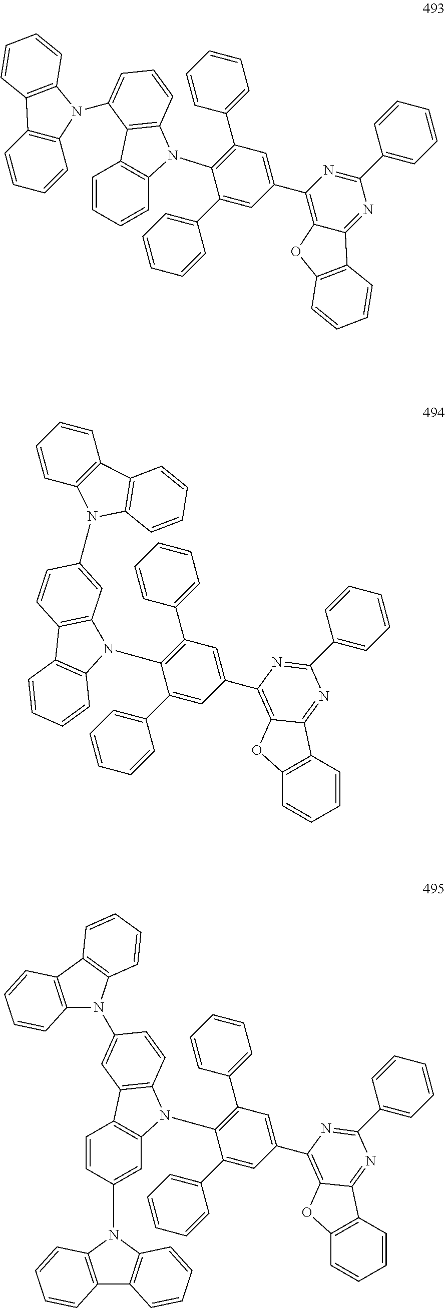 [SMILES: c1ccc(-c2nc(-c3cc(-c4ccccc4)c(-n4c5ccccc5c5c(-n6c7ccccc7c7ccccc76)cccc54)c(-c4ccccc4)c3)c3oc4ccccc4c3n2)cc1 ; c1ccc(-c2nc(-c3cc(-c4ccccc4)c(-n4c5ccccc5c5ccc(-n6c7ccccc7c7ccccc76)cc54)c(-c4ccccc4)c3)c3oc4ccccc4c3n2)cc1 ; c1ccc(-c2nc(-c3cc(-c4ccccc4)c(-n4c5ccc(-n6c7ccccc7c7ccccc76)cc5c5ccc(-n6c7ccccc7c7ccccc76)cc54)c(-c4ccccc4)c3)c3oc4ccccc4c3n2)cc1]

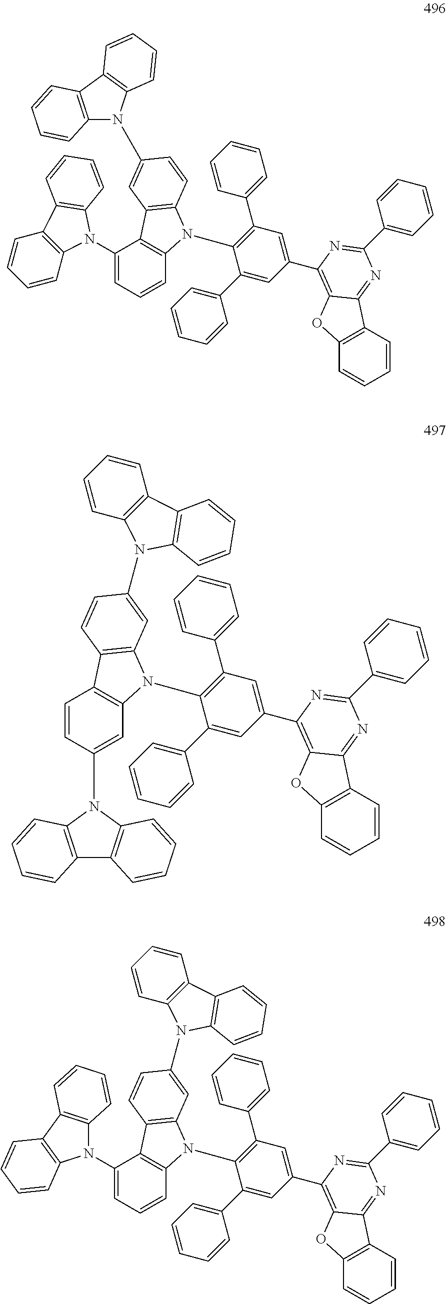 [SMILES: c1ccc(-c2nc(-c3cc(-c4ccccc4)c(-n4c5ccc(-n6c7ccccc7c7ccccc76)cc5c5c(-n6c7ccccc7c7ccccc76)cccc54)c(-c4ccccc4)c3)c3oc4ccccc4c3n2)cc1 ; c1ccc(-c2nc(-c3cc(-c4ccccc4)c(-n4c5cc(-n6c7ccccc7c7ccccc76)ccc5c5ccc(-n6c7ccccc7c7ccccc76)cc54)c(-c4ccccc4)c3)c3oc4ccccc4c3n2)cc1 ; c1ccc(-c2nc(-c3cc(-c4ccccc4)c(-n4c5cc(-n6c7ccccc7c7ccccc76)ccc5c5c(-n6c7ccccc7c7ccccc76)cccc54)c(-c4ccccc4)c3)c3oc4ccccc4c3n2)cc1]

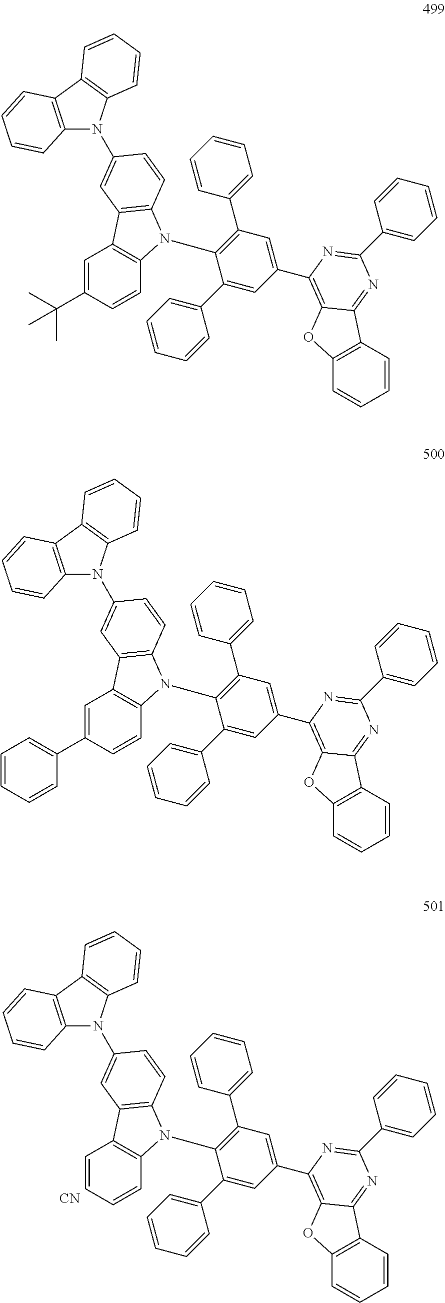

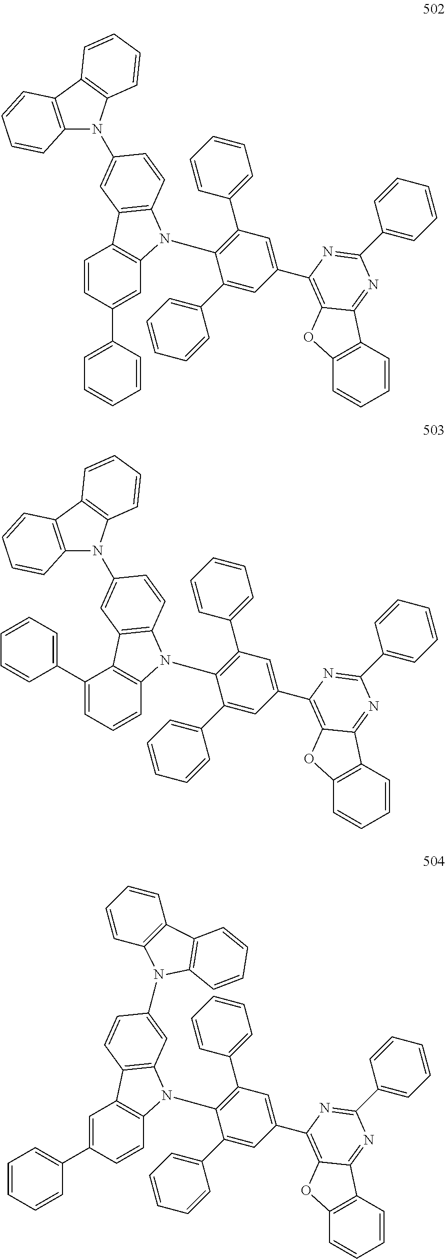 [SMILES: c1ccc(-c2ccc3c4cc(-n5c6ccccc6c6ccccc65)ccc4n(-c4c(-c5ccccc5)cc(-c5nc(-c6ccccc6)nc6c5oc5ccccc56)cc4-c4ccccc4)c3c2)cc1 ; c1ccc(-c2nc(-c3cc(-c4ccccc4)c(-n4c5ccc(-n6c7ccccc7c7ccccc76)cc5c5c(-c6ccccc6)cccc54)c(-c4ccccc4)c3)c3oc4ccccc4c3n2)cc1 ; c1ccc(-c2ccc3c(c2)c2ccc(-n4c5ccccc5c5ccccc54)cc2n3-c2c(-c3ccccc3)cc(-c3nc(-c4ccccc4)nc4c3oc3ccccc34)cc2-c2ccccc2)cc1]

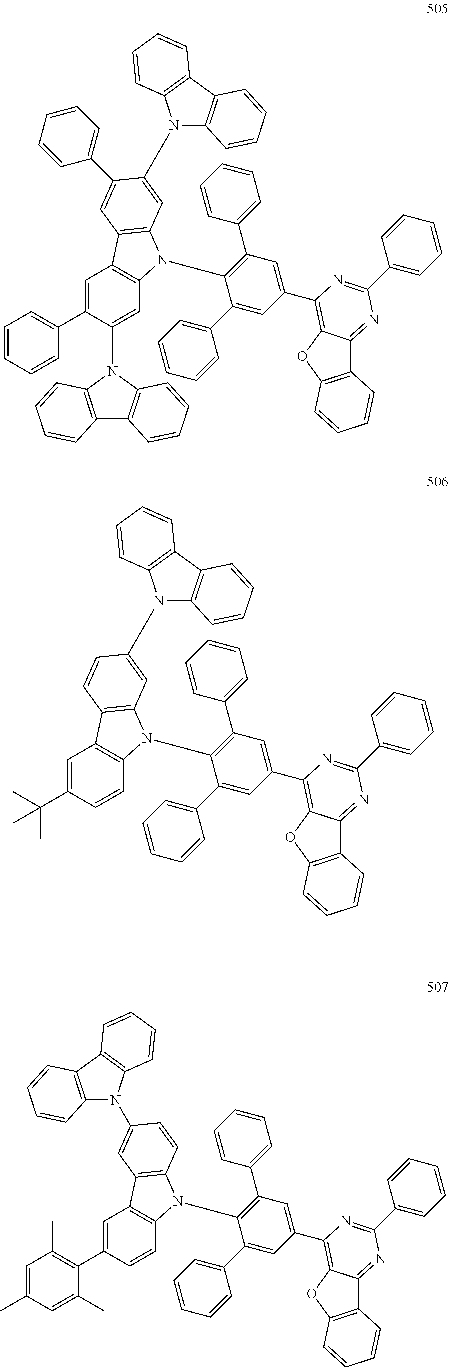 [SMILES: c1ccc(-c2nc(-c3cc(-c4ccccc4)c(-n4c5cc(-n6c7ccccc7c7ccccc76)c(-c6ccccc6)cc5c5cc(-c6ccccc6)c(-n6c7ccccc7c7ccccc76)cc54)c(-c4ccccc4)c3)c3oc4ccccc4c3n2)cc1 ; CC(C)(C)c1ccc2c(c1)c1ccc(-n3c4ccccc4c4ccccc43)cc1n2-c1c(-c2ccccc2)cc(-c2nc(-c3ccccc3)nc3c2oc2ccccc23)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccccc4)nc4c3oc3ccccc34)cc2-c2ccccc2)c(C)c1]

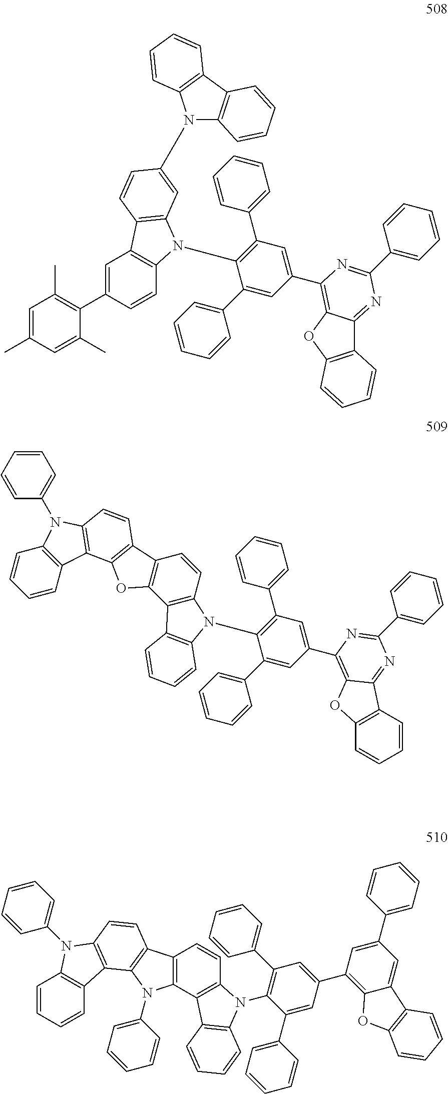 [SMILES: Cc1cc(C)c(-c2ccc3c(c2)c2ccc(-n4c5ccccc5c5ccccc54)cc2n3-c2c(-c3ccccc3)cc(-c3nc(-c4ccccc4)nc4c3oc3ccccc34)cc2-c2ccccc2)c(C)c1 ; c1ccc(-c2nc(-c3cc(-c4ccccc4)c(-n4c5ccccc5c5c6oc7c(ccc8c7c7ccccc7n8-c7ccccc7)c6ccc54)c(-c4ccccc4)c3)c3oc4ccccc4c3n2)cc1 ; c1ccc(-c2cc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)c3oc4ccccc4c3c2)cc1]

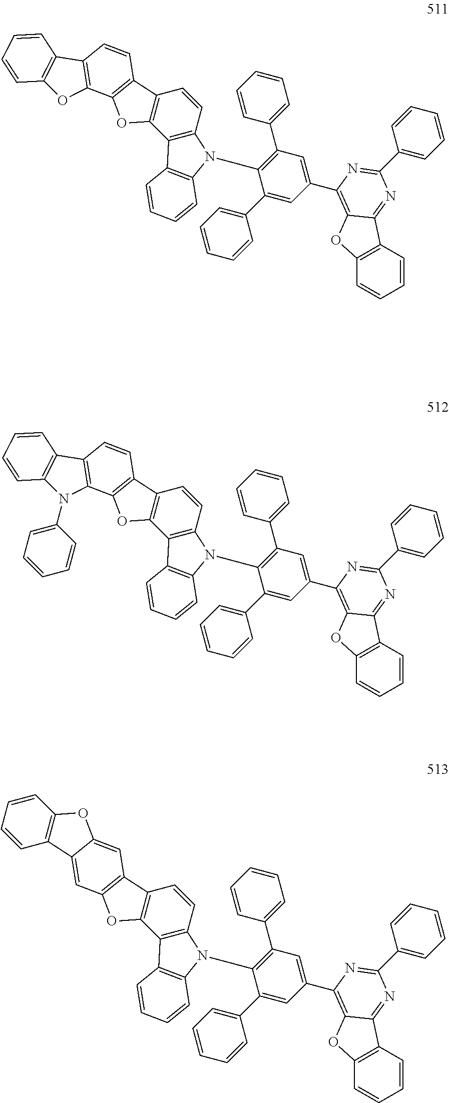 [SMILES: c1ccc(-c2nc(-c3cc(-c4ccccc4)c(-n4c5ccccc5c5c6oc7c(ccc8c9ccccc9oc87)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(ccc8c9ccccc9n(-c9ccccc9)c87)c6ccc54)c(-c4ccccc4)c3)c3oc4ccccc4c3n2)cc1 ; c1ccc(-c2nc(-c3cc(-c4ccccc4)c(-n4c5ccccc5c5c6oc7cc8c(cc7c6ccc54)oc4ccccc48)c(-c4ccccc4)c3)c3oc4ccccc4c3n2)cc1]

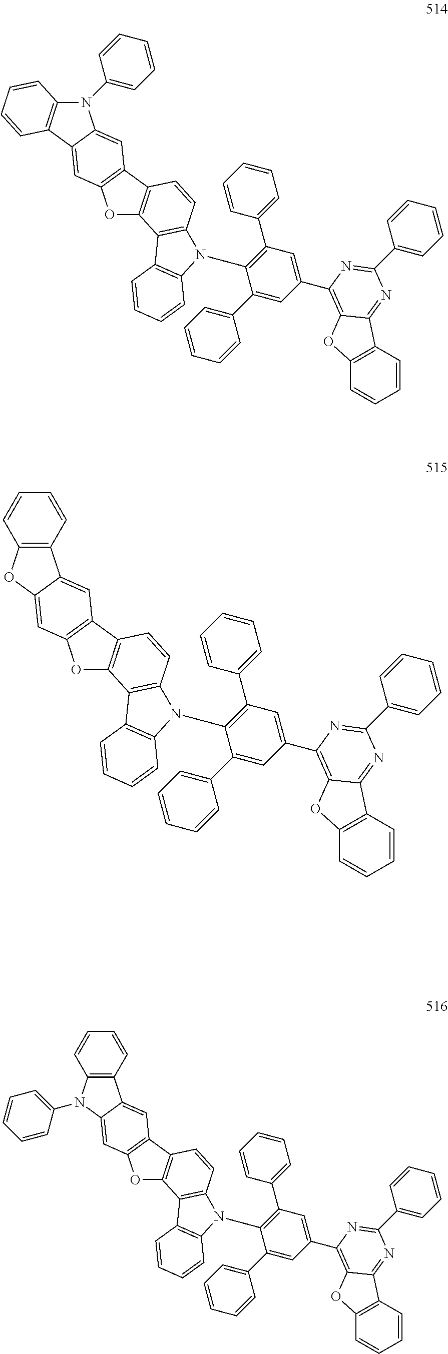 [SMILES: c1ccc(-c2nc(-c3cc(-c4ccccc4)c(-n4c5ccccc5c5c6oc7cc8c9ccccc9n(-c9ccccc9)c8cc7c6ccc54)c(-c4ccccc4)c3)c3oc4ccccc4c3n2)cc1 ; c1ccc(-c2nc(-c3cc(-c4ccccc4)c(-n4c5ccccc5c5c6oc7cc8oc9ccccc9c8cc7c6ccc54)c(-c4ccccc4)c3)c3oc4ccccc4c3n2)cc1 ; c1ccc(-c2nc(-c3cc(-c4ccccc4)c(-n4c5ccccc5c5c6oc7cc8c(cc7c6ccc54)c4ccccc4n8-c4ccccc4)c(-c4ccccc4)c3)c3oc4ccccc4c3n2)cc1]

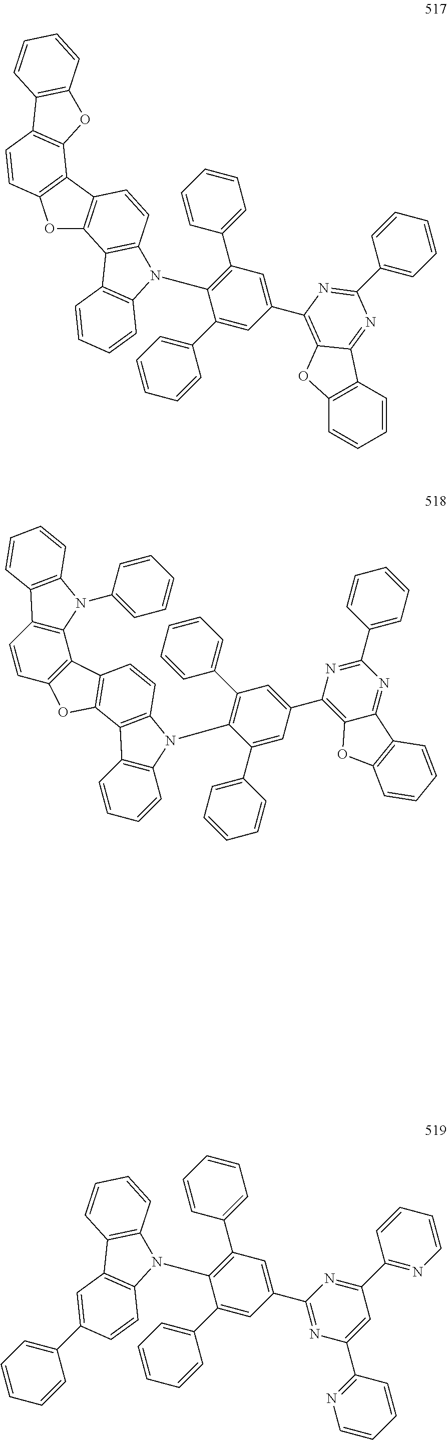 [SMILES: c1ccc(-c2nc(-c3cc(-c4ccccc4)c(-n4c5ccccc5c5c6oc7ccc8c9ccccc9oc8c7c6ccc54)c(-c4ccccc4)c3)c3oc4ccccc4c3n2)cc1 ; c1ccc(-c2nc(-c3cc(-c4ccccc4)c(-n4c5ccccc5c5c6oc7ccc8c9ccccc9n(-c9ccccc9)c8c7c6ccc54)c(-c4ccccc4)c3)c3oc4ccccc4c3n2)cc1 ; c1ccc(-c2ccc3c(c2)c2ccccc2n3-c2c(-c3ccccc3)cc(-c3nc(-c4ccccn4)cc(-c4ccccn4)n3)cc2-c2ccccc2)cc1]

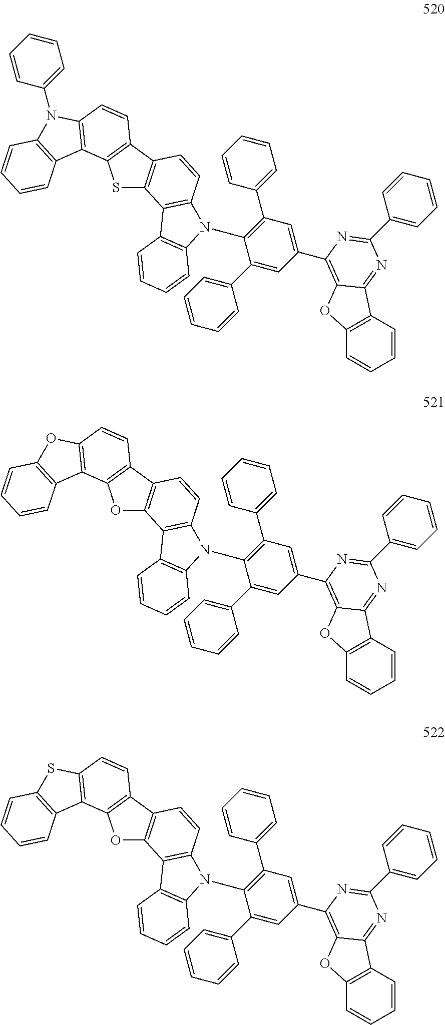 [SMILES: c1ccc(-c2nc(-c3cc(-c4ccccc4)c(-n4c5ccccc5c5c6sc7c(ccc8c7c7ccccc7n8-c7ccccc7)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(ccc8oc9ccccc9c87)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(ccc8sc9ccccc9c87)c6ccc54)c(-c4ccccc4)c3)c3oc4ccccc4c3n2)cc1]

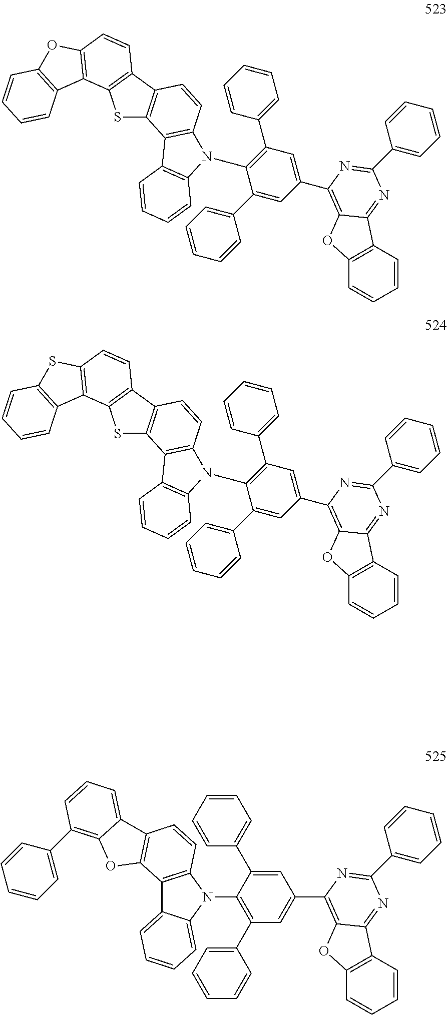 [SMILES: c1ccc(-c2nc(-c3cc(-c4ccccc4)c(-n4c5ccccc5c5c6sc7c(ccc8oc9ccccc9c87)c6ccc54)c(-c4ccccc4)c3)c3oc4ccccc4c3n2)cc1 ; c1ccc(-c2nc(-c3cc(-c4ccccc4)c(-n4c5ccccc5c5c6sc7c(ccc8sc9ccccc9c87)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(-c8ccccc8)cccc7c6ccc54)c(-c4ccccc4)c3)c3oc4ccccc4c3n2)cc1]

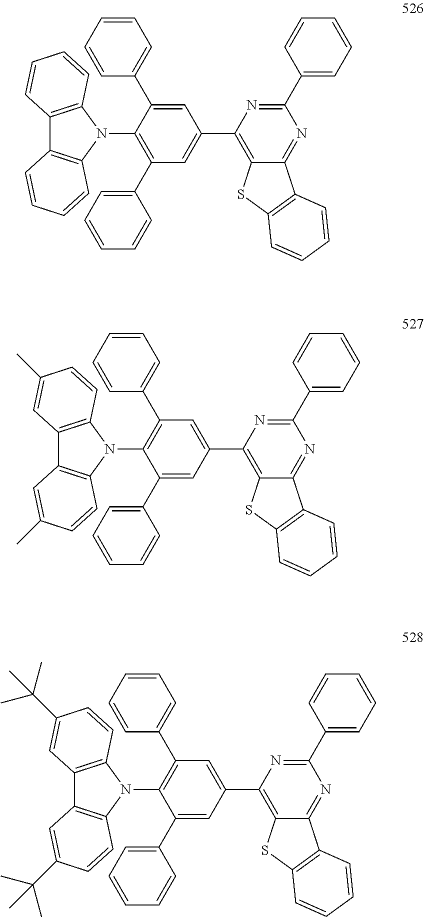 [SMILES: c1ccc(-c2nc(-c3cc(-c4ccccc4)c(-n4c5ccccc5c5ccccc54)c(-c4ccccc4)c3)c3sc4ccccc4c3n2)cc1 ; Cc1ccc2c(c1)c1cc(C)ccc1n2-c1c(-c2ccccc2)cc(-c2nc(-c3ccccc3)nc3c2sc2ccccc23)cc1-c1ccccc1 ; CC(C)(C)c1ccc2c(c1)c1cc(C(C)(C)C)ccc1n2-c1c(-c2ccccc2)cc(-c2nc(-c3ccccc3)nc3c2sc2ccccc23)cc1-c1ccccc1]

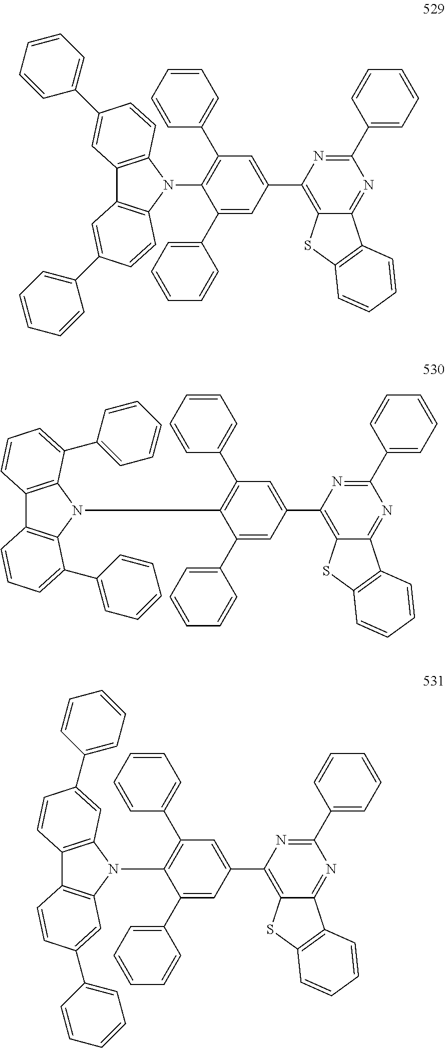 [SMILES: c1ccc(-c2ccc3c(c2)c2cc(-c4ccccc4)ccc2n3-c2c(-c3ccccc3)cc(-c3nc(-c4ccccc4)nc4c3sc3ccccc34)cc2-c2ccccc2)cc1 ; c1ccc(-c2nc(-c3cc(-c4ccccc4)c(-n4c5c(-c6ccccc6)cccc5c5cccc(-c6ccccc6)c54)c(-c4ccccc4)c3)c3sc4ccccc4c3n2)cc1 ; c1ccc(-c2ccc3c4ccc(-c5ccccc5)cc4n(-c4c(-c5ccccc5)cc(-c5nc(-c6ccccc6)nc6c5sc5ccccc56)cc4-c4ccccc4)c3c2)cc1]

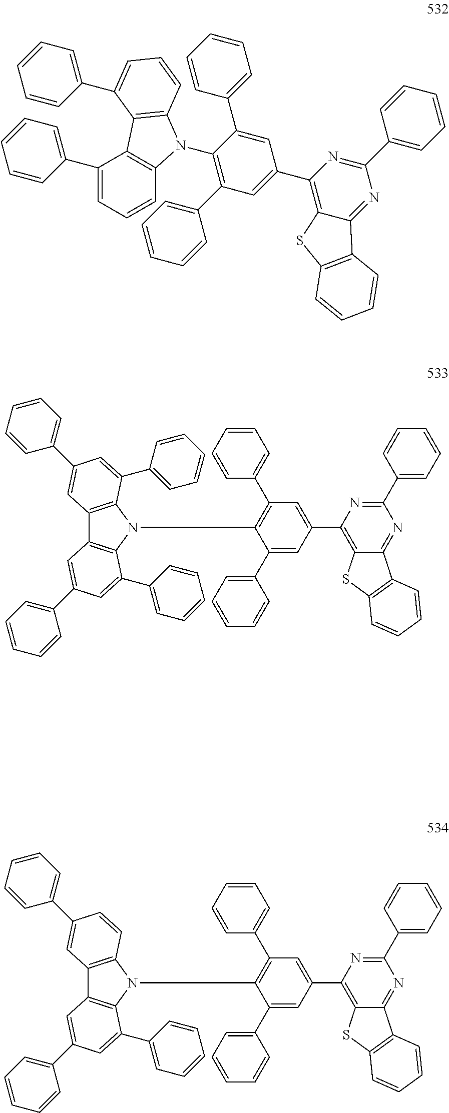 [SMILES: c1ccc(-c2nc(-c3cc(-c4ccccc4)c(-n4c5cccc(-c6ccccc6)c5c5c(-c6ccccc6)cccc54)c(-c4ccccc4)c3)c3sc4ccccc4c3n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)nc4c3sc3ccccc34)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccccc4)nc4c3sc3ccccc34)cc2-c2ccccc2)cc1]

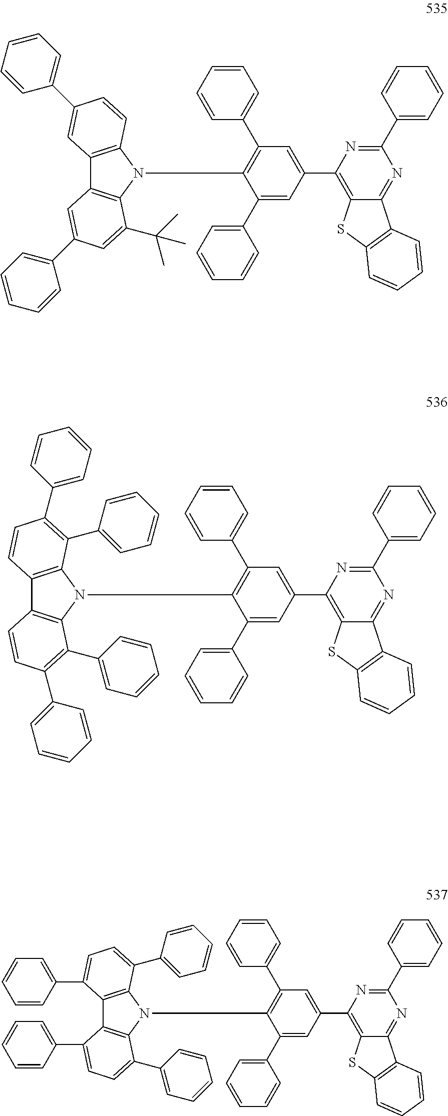 [SMILES: CC(C)(C)c1cc(-c2ccccc2)cc2c3cc(-c4ccccc4)ccc3n(-c3c(-c4ccccc4)cc(-c4nc(-c5ccccc5)nc5c4sc4ccccc45)cc3-c3ccccc3)c12 ; c1ccc(-c2nc(-c3cc(-c4ccccc4)c(-n4c5c(-c6ccccc6)c(-c6ccccc6)ccc5c5ccc(-c6ccccc6)c(-c6ccccc6)c54)c(-c4ccccc4)c3)c3sc4ccccc4c3n2)cc1 ; c1ccc(-c2nc(-c3cc(-c4ccccc4)c(-n4c5c(-c6ccccc6)ccc(-c6ccccc6)c5c5c(-c6ccccc6)ccc(-c6ccccc6)c54)c(-c4ccccc4)c3)c3sc4ccccc4c3n2)cc1]

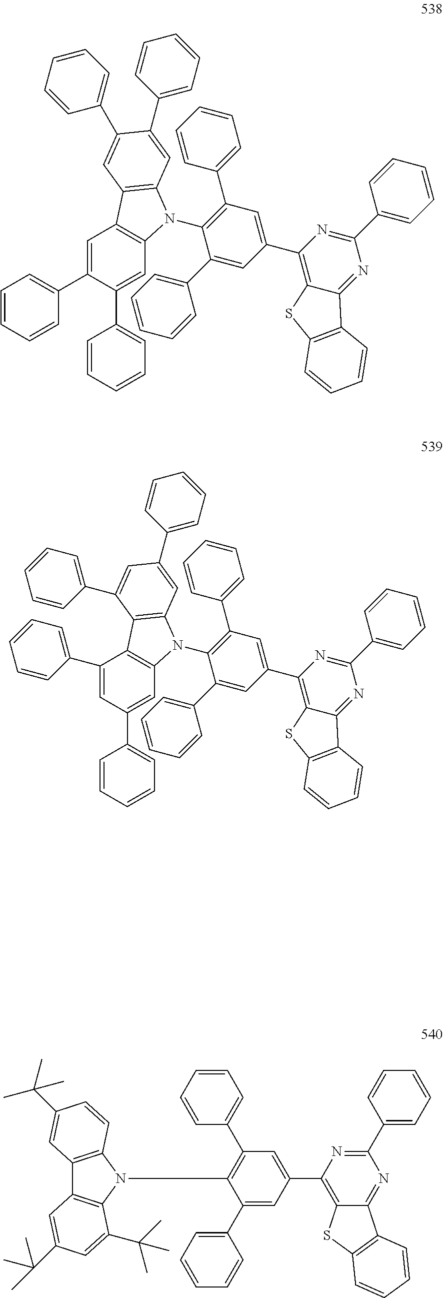 [SMILES: c1ccc(-c2nc(-c3cc(-c4ccccc4)c(-n4c5cc(-c6ccccc6)c(-c6ccccc6)cc5c5cc(-c6ccccc6)c(-c6ccccc6)cc54)c(-c4ccccc4)c3)c3sc4ccccc4c3n2)cc1 ; c1ccc(-c2cc(-c3ccccc3)c3c4c(-c5ccccc5)cc(-c5ccccc5)cc4n(-c4c(-c5ccccc5)cc(-c5nc(-c6ccccc6)nc6c5sc5ccccc56)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)nc3c2sc2ccccc23)cc1-c1ccccc1]

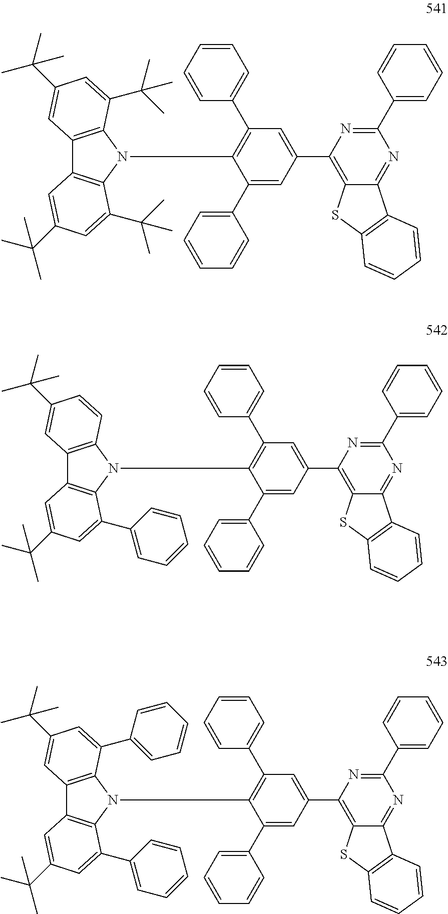 [SMILES: CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)nc3c2sc2ccccc23)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccccc3)nc3c2sc2ccccc23)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccccc3)nc3c2sc2ccccc23)cc1-c1ccccc1]

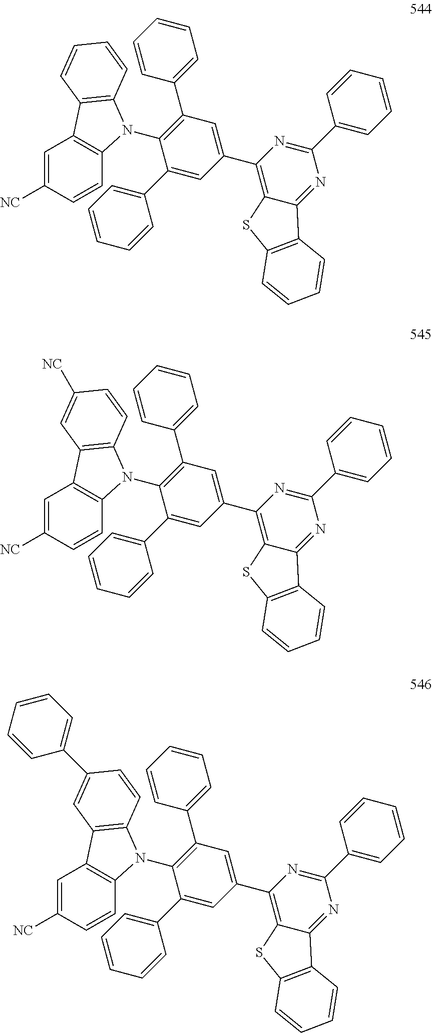 [SMILES: N#Cc1ccc2c(c1)c1ccccc1n2-c1c(-c2ccccc2)cc(-c2nc(-c3ccccc3)nc3c2sc2ccccc23)cc1-c1ccccc1 ; N#Cc1ccc2c(c1)c1cc(C#N)ccc1n2-c1c(-c2ccccc2)cc(-c2nc(-c3ccccc3)nc3c2sc2ccccc23)cc1-c1ccccc1 ; N#Cc1ccc2c(c1)c1cc(-c3ccccc3)ccc1n2-c1c(-c2ccccc2)cc(-c2nc(-c3ccccc3)nc3c2sc2ccccc23)cc1-c1ccccc1]

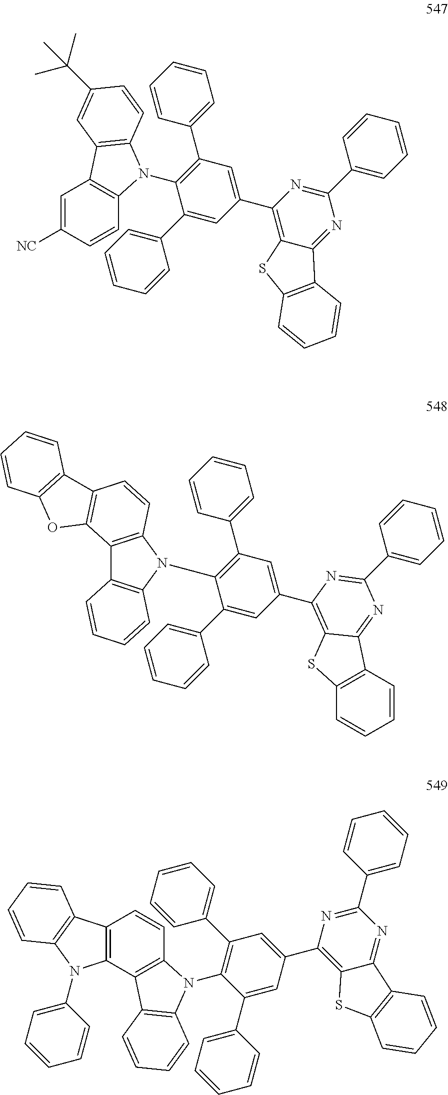 [SMILES: CC(C)(C)c1ccc2c(c1)c1cc(C#N)ccc1n2-c1c(-c2ccccc2)cc(-c2nc(-c3ccccc3)nc3c2sc2ccccc23)cc1-c1ccccc1 ; c1ccc(-c2nc(-c3cc(-c4ccccc4)c(-n4c5ccccc5c5c6oc7ccccc7c6ccc54)c(-c4ccccc4)c3)c3sc4ccccc4c3n2)cc1 ; c1ccc(-c2nc(-c3cc(-c4ccccc4)c(-n4c5ccccc5c5c4ccc4c6ccccc6n(-c6ccccc6)c45)c(-c4ccccc4)c3)c3sc4ccccc4c3n2)cc1]

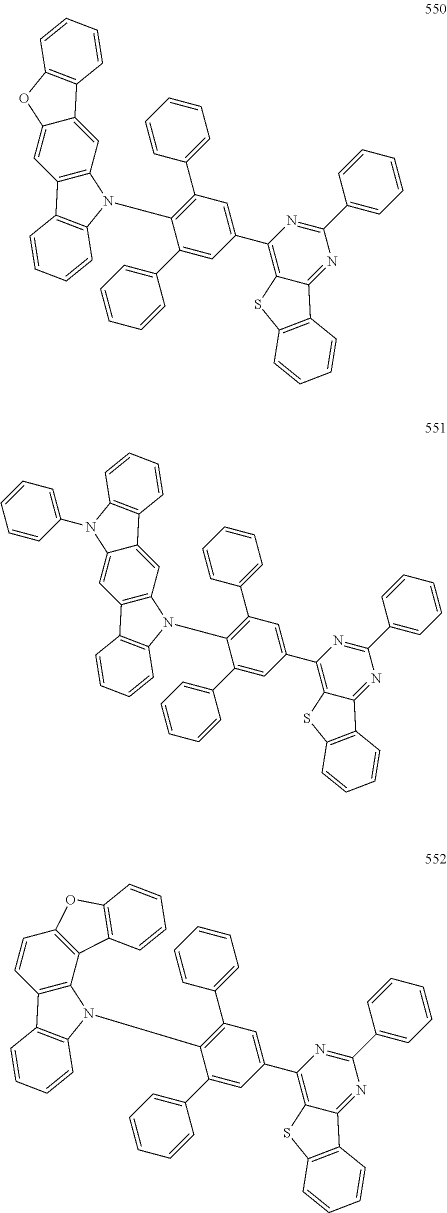 [SMILES: c1ccc(-c2nc(-c3cc(-c4ccccc4)c(-n4c5ccccc5c5cc6oc7ccccc7c6cc54)c(-c4ccccc4)c3)c3sc4ccccc4c3n2)cc1 ; c1ccc(-c2nc(-c3cc(-c4ccccc4)c(-n4c5ccccc5c5cc6c(cc54)c4ccccc4n6-c4ccccc4)c(-c4ccccc4)c3)c3sc4ccccc4c3n2)cc1 ; c1ccc(-c2nc(-c3cc(-c4ccccc4)c(-n4c5ccccc5c5ccc6oc7ccccc7c6c54)c(-c4ccccc4)c3)c3sc4ccccc4c3n2)cc1]

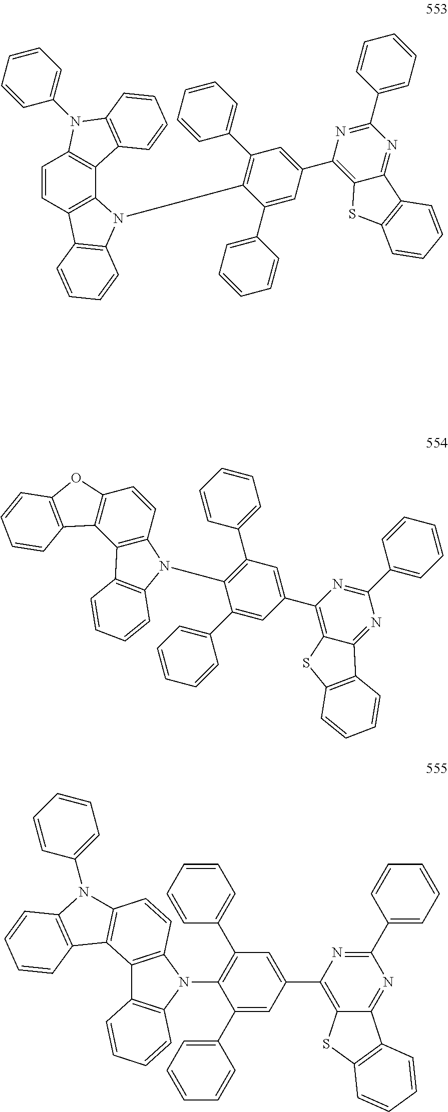 [SMILES: c1ccc(-c2nc(-c3cc(-c4ccccc4)c(-n4c5ccccc5c5ccc6c(c7ccccc7n6-c6ccccc6)c54)c(-c4ccccc4)c3)c3sc4ccccc4c3n2)cc1 ; c1ccc(-c2nc(-c3cc(-c4ccccc4)c(-n4c5ccccc5c5c6c(ccc54)oc4ccccc46)c(-c4ccccc4)c3)c3sc4ccccc4c3n2)cc1 ; c1ccc(-c2nc(-c3cc(-c4ccccc4)c(-n4c5ccccc5c5c6c7ccccc7n(-c7ccccc7)c6ccc54)c(-c4ccccc4)c3)c3sc4ccccc4c3n2)cc1]

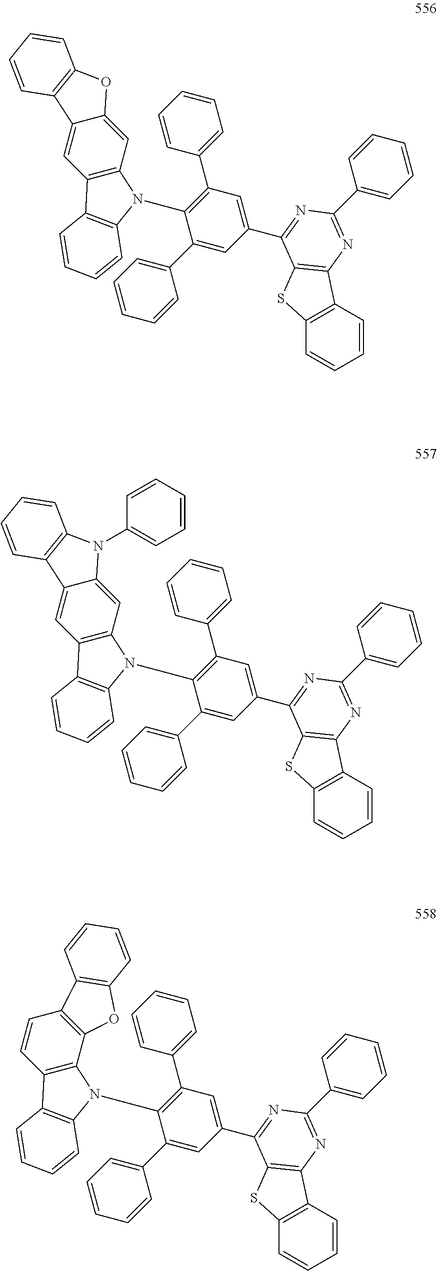 [SMILES: c1ccc(-c2nc(-c3cc(-c4ccccc4)c(-n4c5ccccc5c5cc6c(cc54)oc4ccccc46)c(-c4ccccc4)c3)c3sc4ccccc4c3n2)cc1 ; c1ccc(-c2nc(-c3cc(-c4ccccc4)c(-n4c5ccccc5c5cc6c7ccccc7n(-c7ccccc7)c6cc54)c(-c4ccccc4)c3)c3sc4ccccc4c3n2)cc1 ; c1ccc(-c2nc(-c3cc(-c4ccccc4)c(-n4c5ccccc5c5ccc6c7ccccc7oc6c54)c(-c4ccccc4)c3)c3sc4ccccc4c3n2)cc1]

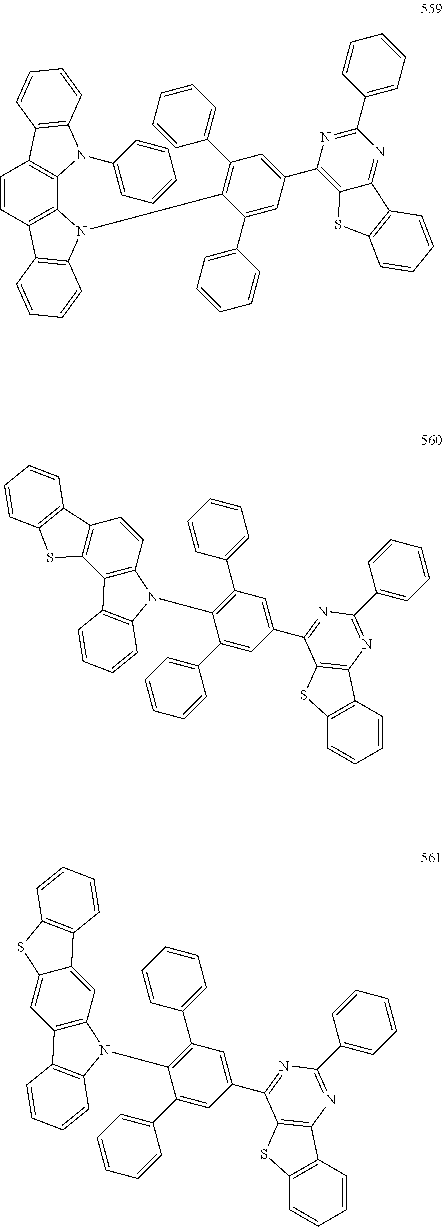 [SMILES: c1ccc(-c2nc(-c3cc(-c4ccccc4)c(-n4c5ccccc5c5ccc6c7ccccc7n(-c7ccccc7)c6c54)c(-c4ccccc4)c3)c3sc4ccccc4c3n2)cc1 ; c1ccc(-c2nc(-c3cc(-c4ccccc4)c(-n4c5ccccc5c5c6sc7ccccc7c6ccc54)c(-c4ccccc4)c3)c3sc4ccccc4c3n2)cc1 ; c1ccc(-c2nc(-c3cc(-c4ccccc4)c(-n4c5ccccc5c5cc6sc7ccccc7c6cc54)c(-c4ccccc4)c3)c3sc4ccccc4c3n2)cc1]

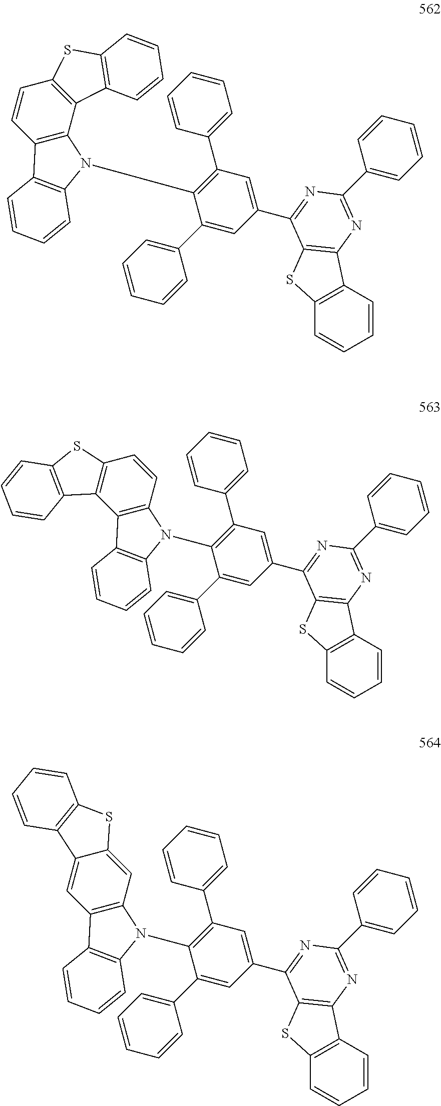 [SMILES: c1ccc(-c2nc(-c3cc(-c4ccccc4)c(-n4c5ccccc5c5ccc6sc7ccccc7c6c54)c(-c4ccccc4)c3)c3sc4ccccc4c3n2)cc1 ; c1ccc(-c2nc(-c3cc(-c4ccccc4)c(-n4c5ccccc5c5c6c(ccc54)sc4ccccc46)c(-c4ccccc4)c3)c3sc4ccccc4c3n2)cc1 ; c1ccc(-c2nc(-c3cc(-c4ccccc4)c(-n4c5ccccc5c5cc6c(cc54)sc4ccccc46)c(-c4ccccc4)c3)c3sc4ccccc4c3n2)cc1]

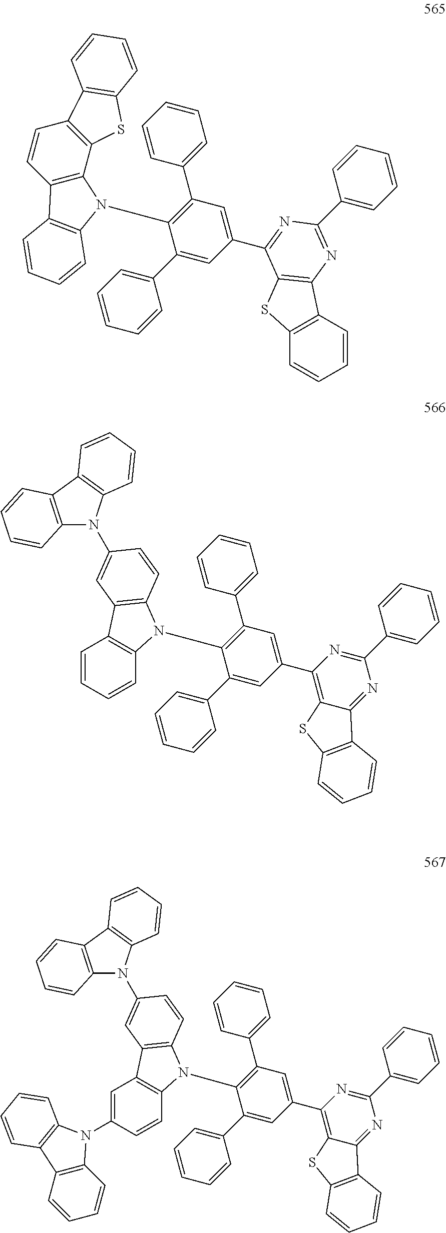 [SMILES: c1ccc(-c2nc(-c3cc(-c4ccccc4)c(-n4c5ccccc5c5ccc6c7ccccc7sc6c54)c(-c4ccccc4)c3)c3sc4ccccc4c3n2)cc1 ; c1ccc(-c2nc(-c3cc(-c4ccccc4)c(-n4c5ccccc5c5cc(-n6c7ccccc7c7ccccc76)ccc54)c(-c4ccccc4)c3)c3sc4ccccc4c3n2)cc1 ; c1ccc(-c2nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)c3sc4ccccc4c3n2)cc1]

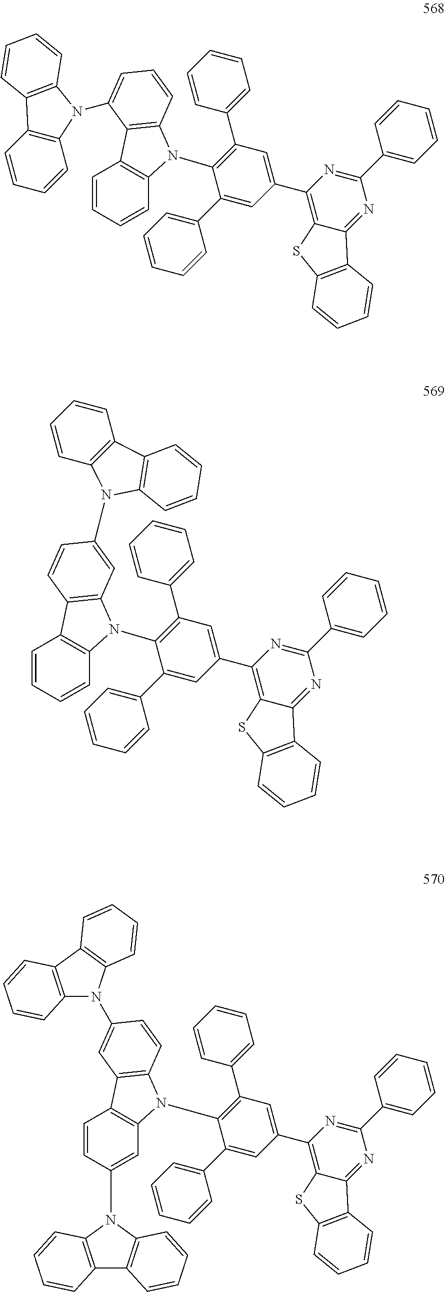 [SMILES: c1ccc(-c2nc(-c3cc(-c4ccccc4)c(-n4c5ccccc5c5c(-n6c7ccccc7c7ccccc76)cccc54)c(-c4ccccc4)c3)c3sc4ccccc4c3n2)cc1 ; c1ccc(-c2nc(-c3cc(-c4ccccc4)c(-n4c5ccccc5c5ccc(-n6c7ccccc7c7ccccc76)cc54)c(-c4ccccc4)c3)c3sc4ccccc4c3n2)cc1 ; c1ccc(-c2nc(-c3cc(-c4ccccc4)c(-n4c5ccc(-n6c7ccccc7c7ccccc76)cc5c5ccc(-n6c7ccccc7c7ccccc76)cc54)c(-c4ccccc4)c3)c3sc4ccccc4c3n2)cc1]

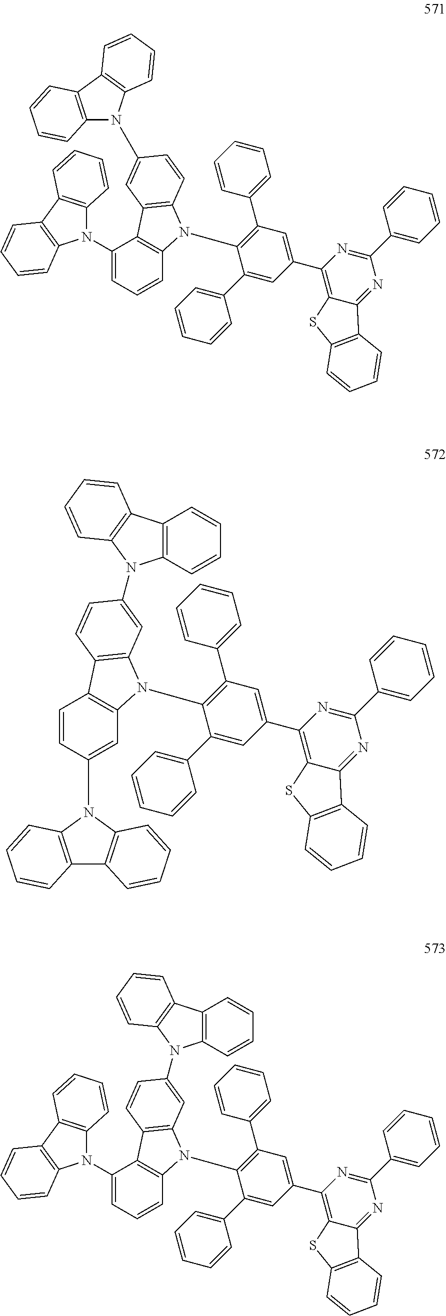 [SMILES: c1ccc(-c2nc(-c3cc(-c4ccccc4)c(-n4c5ccc(-n6c7ccccc7c7ccccc76)cc5c5c(-n6c7ccccc7c7ccccc76)cccc54)c(-c4ccccc4)c3)c3sc4ccccc4c3n2)cc1 ; c1ccc(-c2nc(-c3cc(-c4ccccc4)c(-n4c5cc(-n6c7ccccc7c7ccccc76)ccc5c5ccc(-n6c7ccccc7c7ccccc76)cc54)c(-c4ccccc4)c3)c3sc4ccccc4c3n2)cc1 ; c1ccc(-c2nc(-c3cc(-c4ccccc4)c(-n4c5cc(-n6c7ccccc7c7ccccc76)ccc5c5c(-n6c7ccccc7c7ccccc76)cccc54)c(-c4ccccc4)c3)c3sc4ccccc4c3n2)cc1]

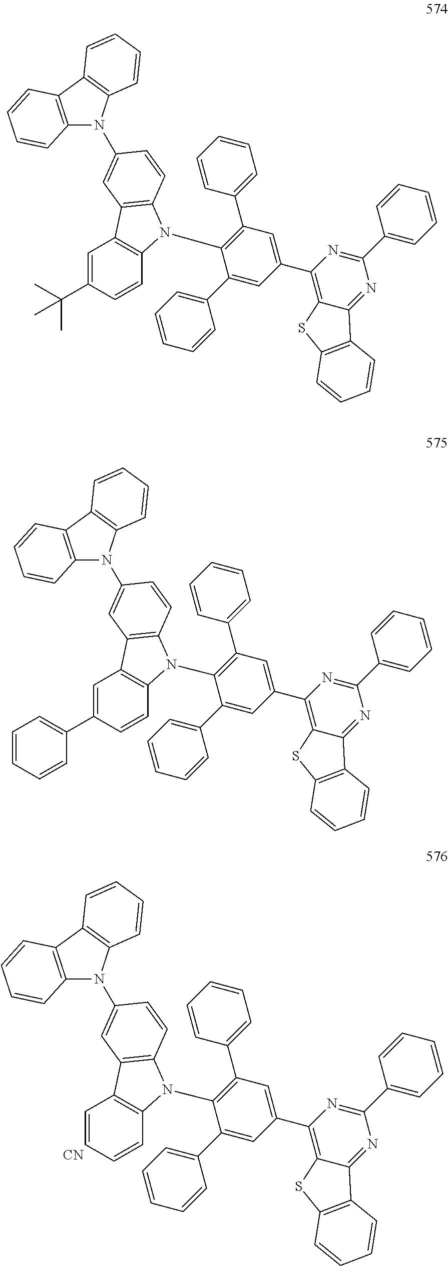

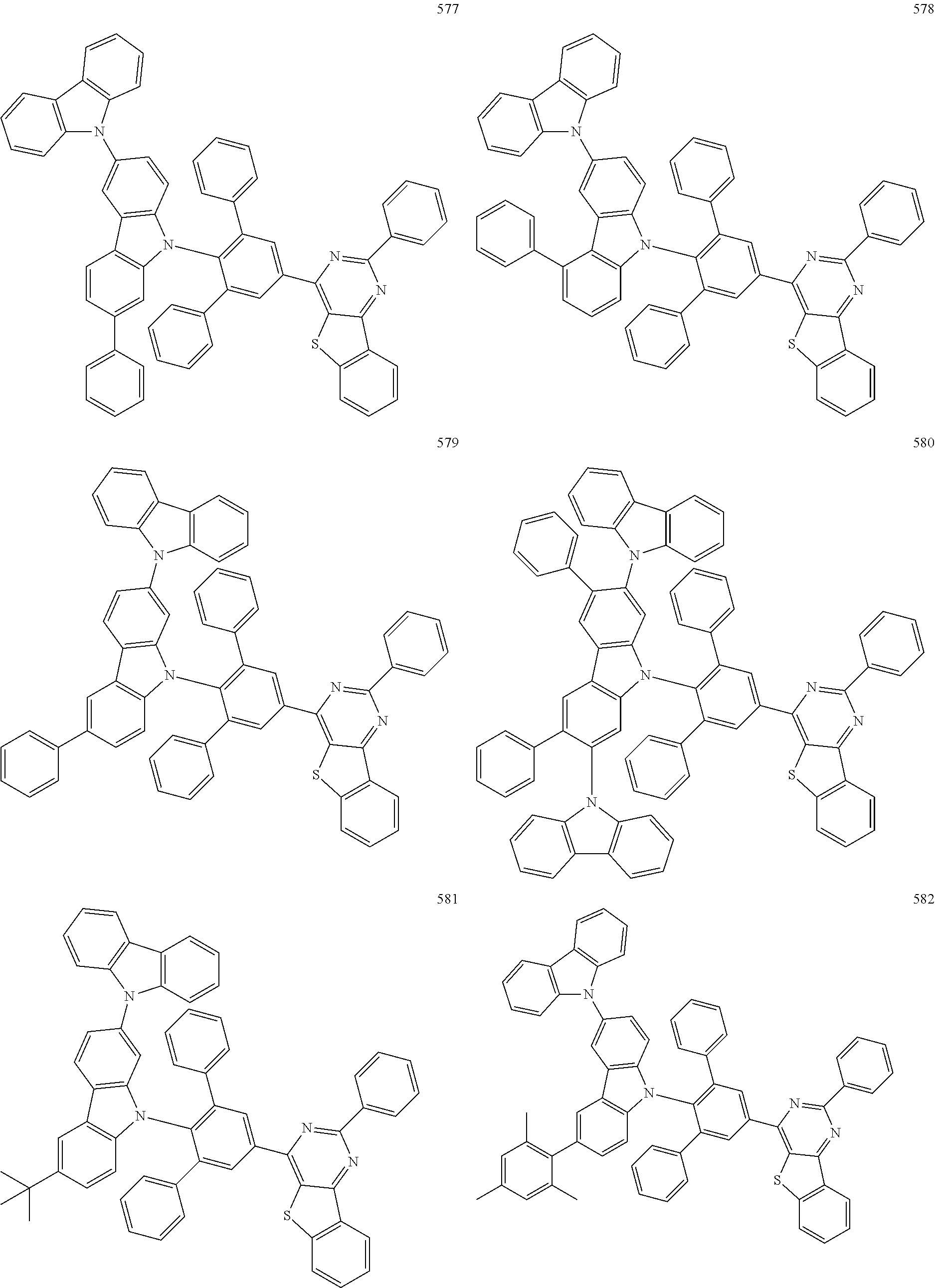

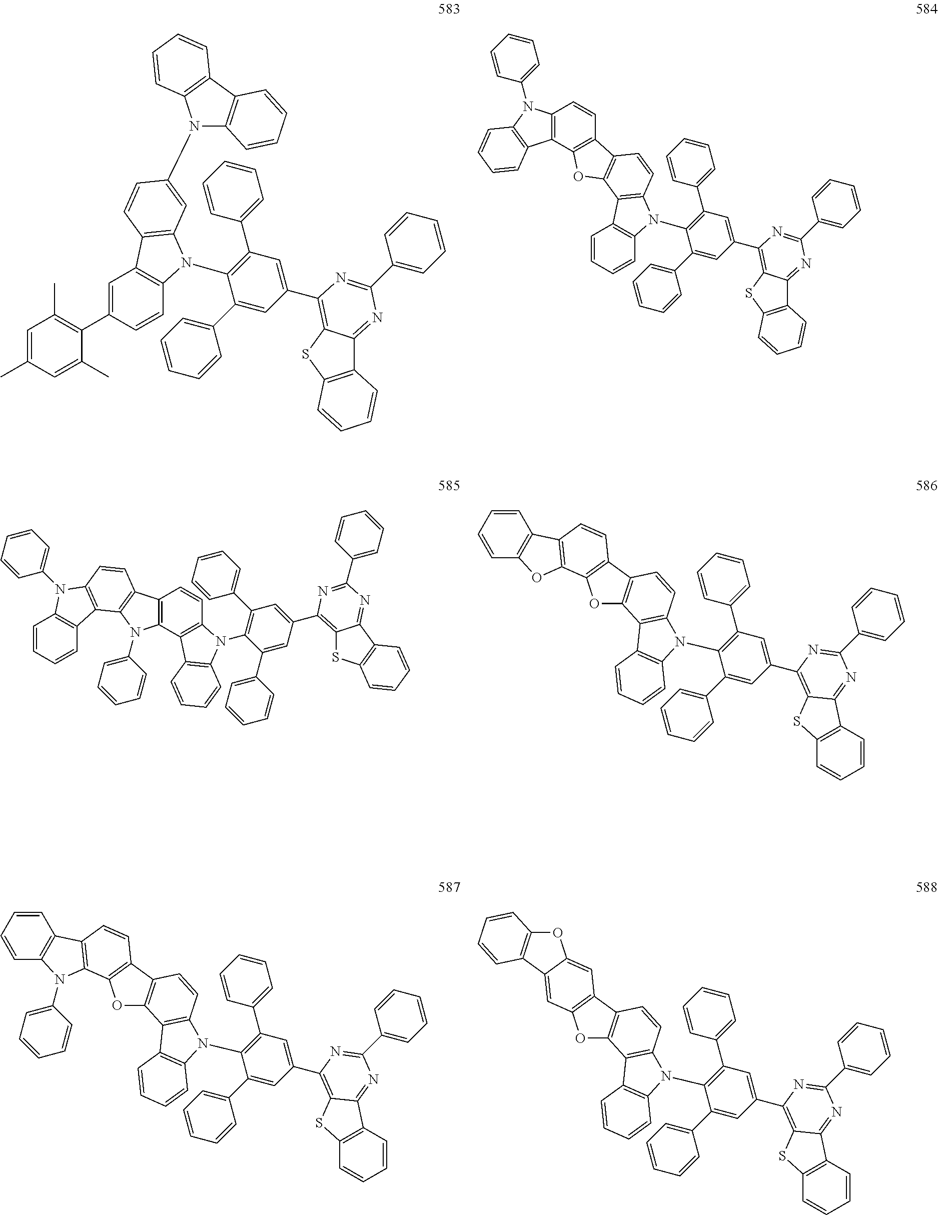

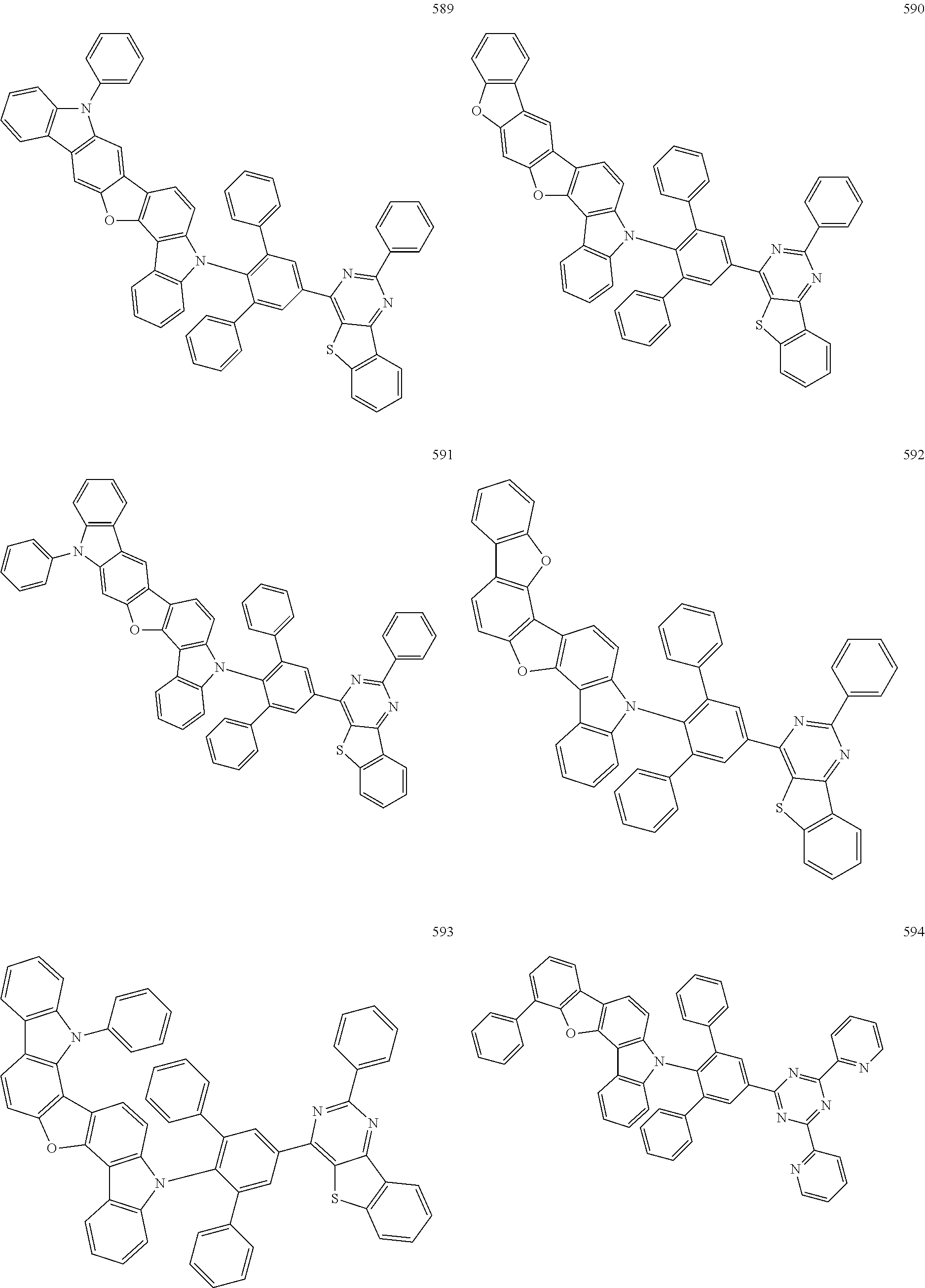

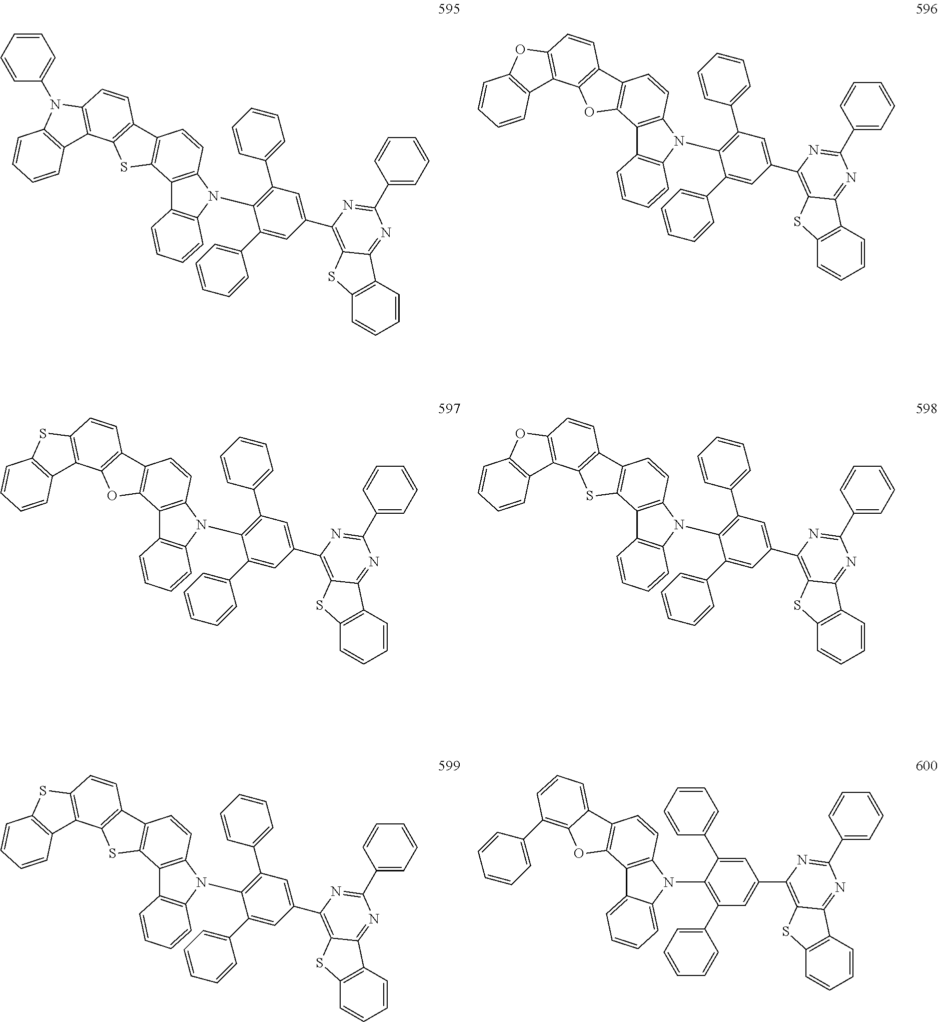

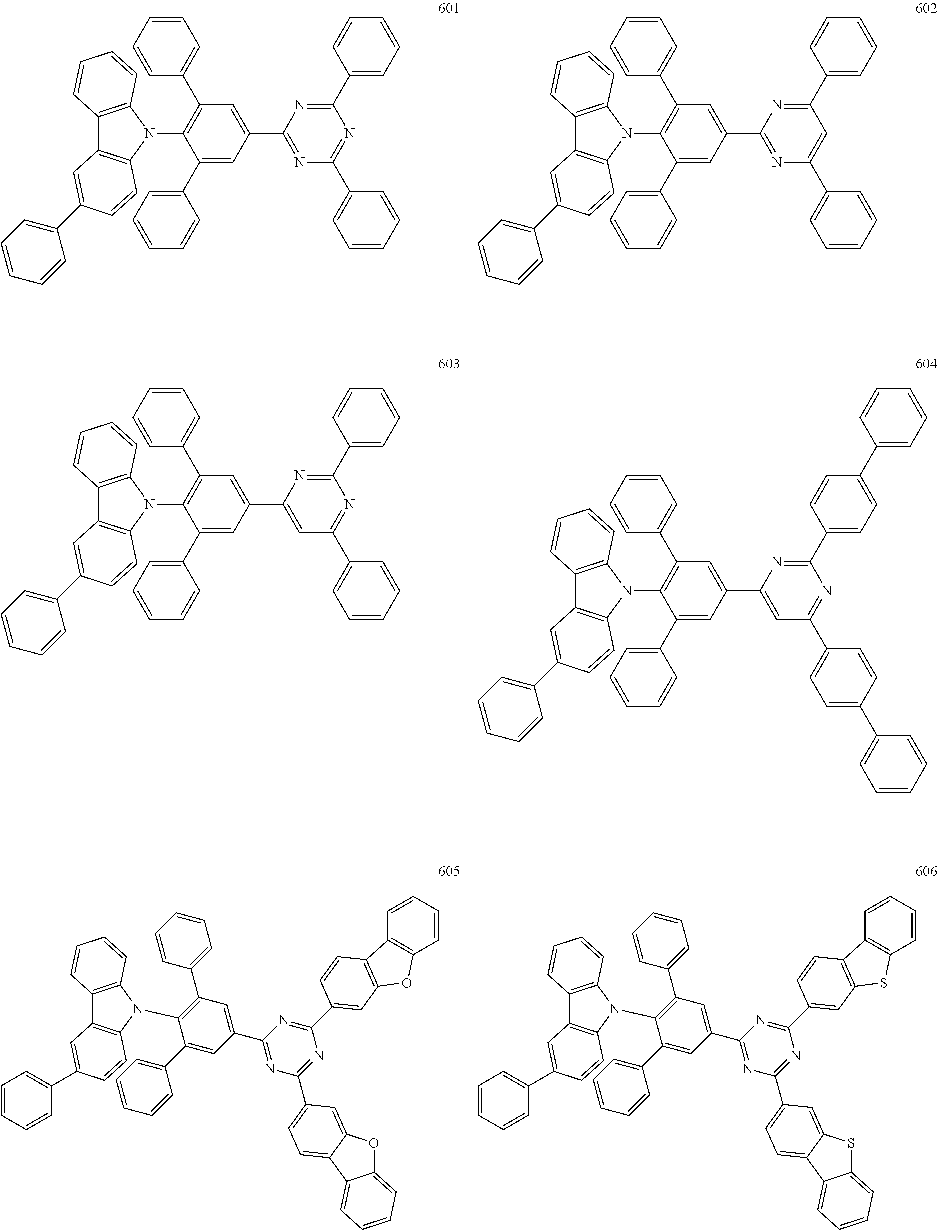

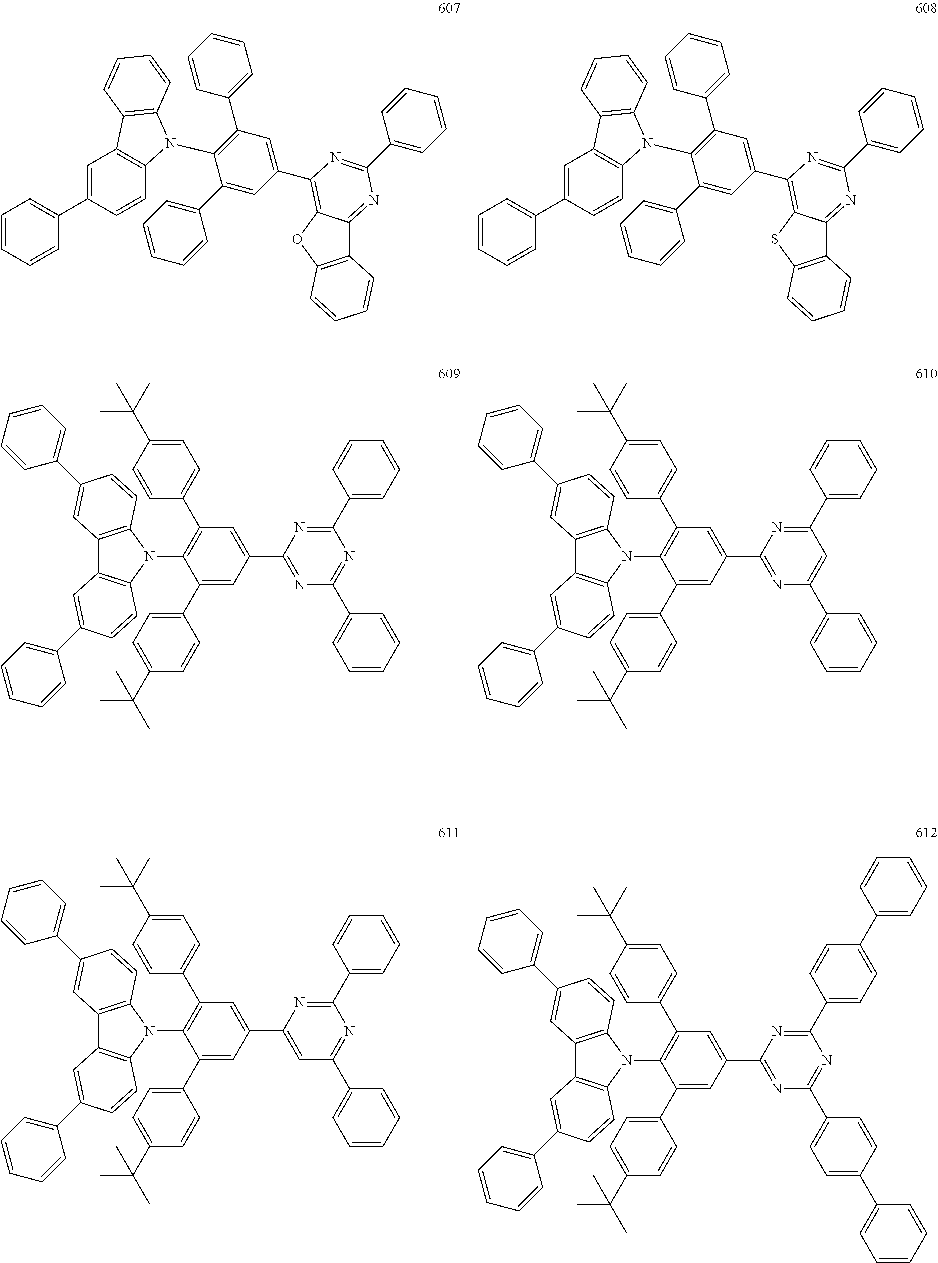

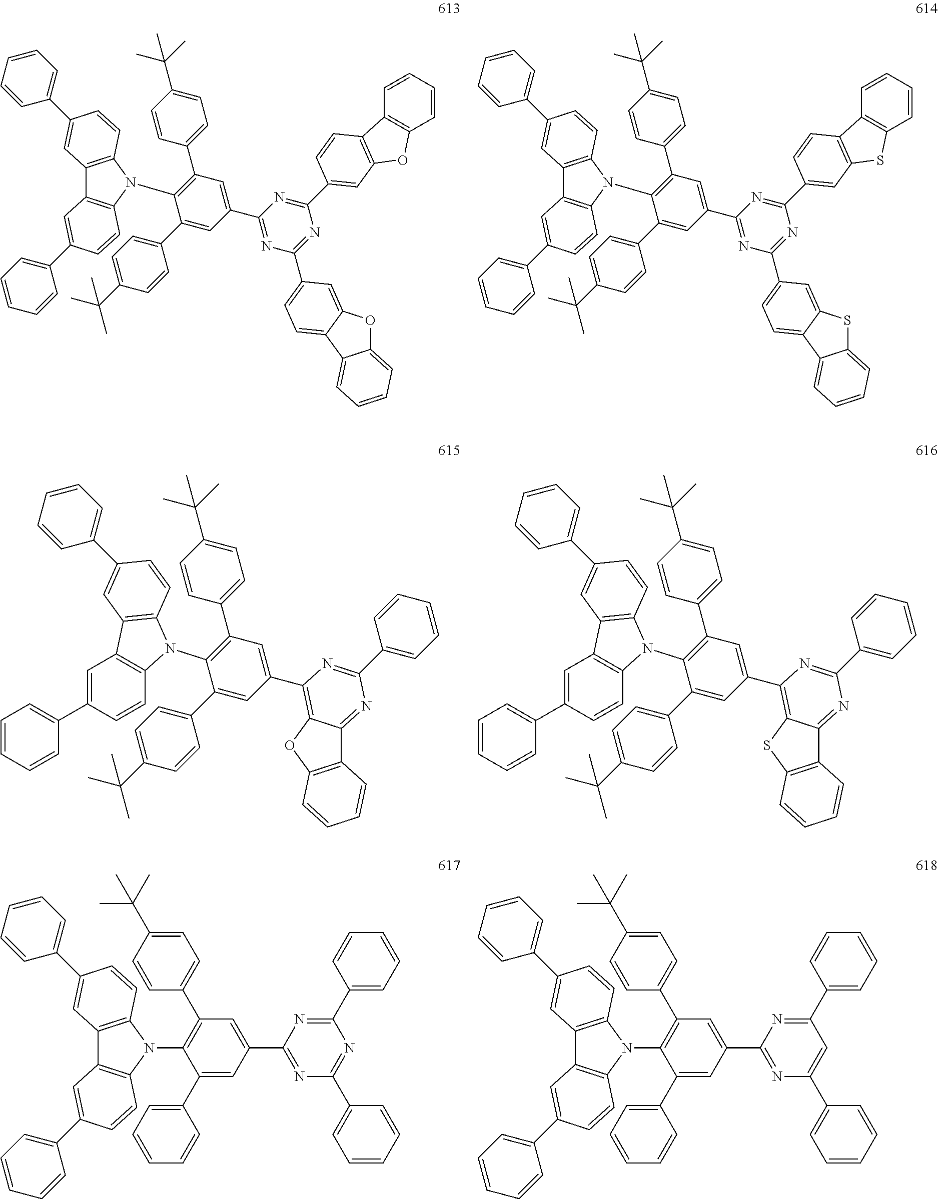

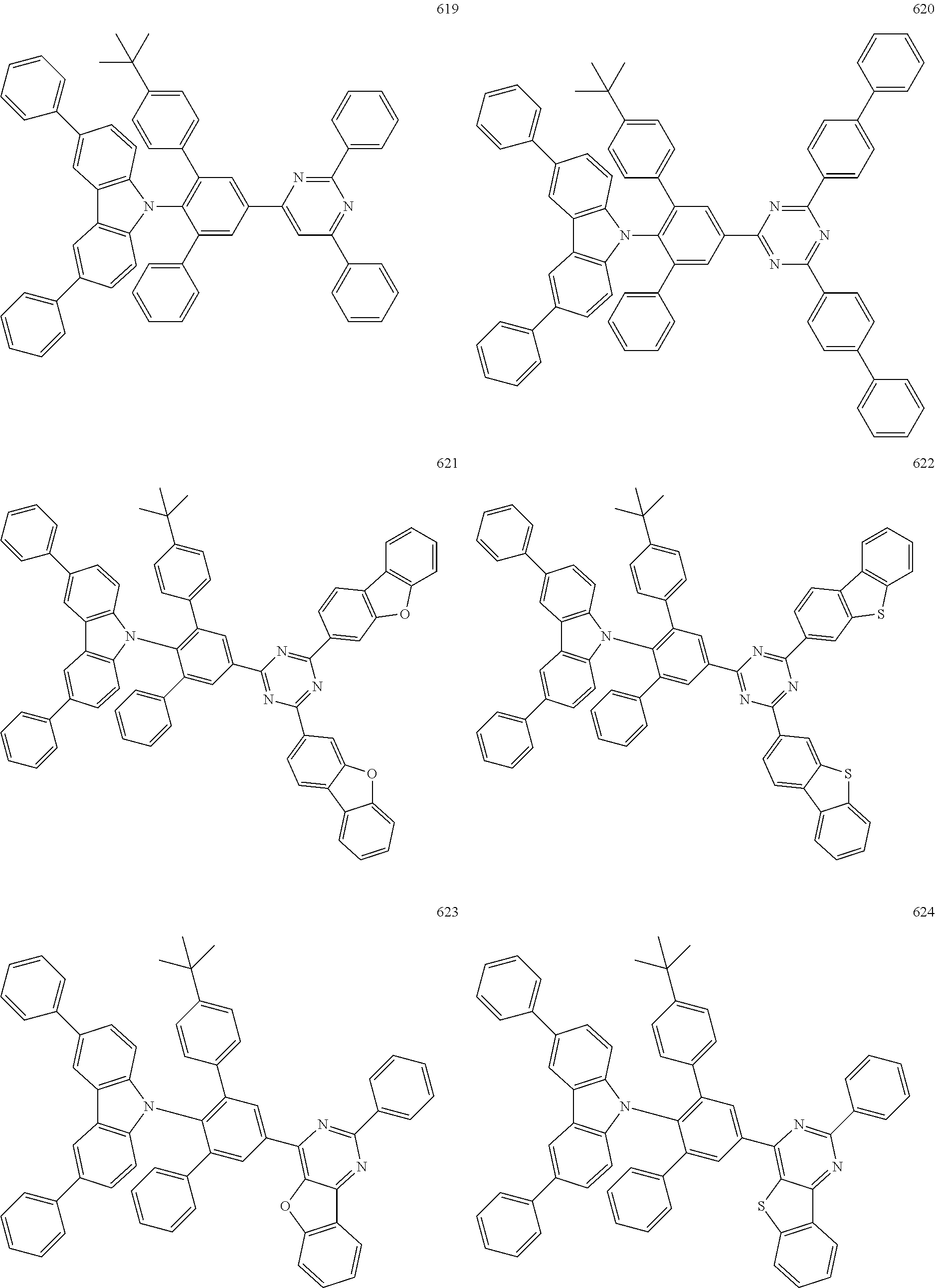

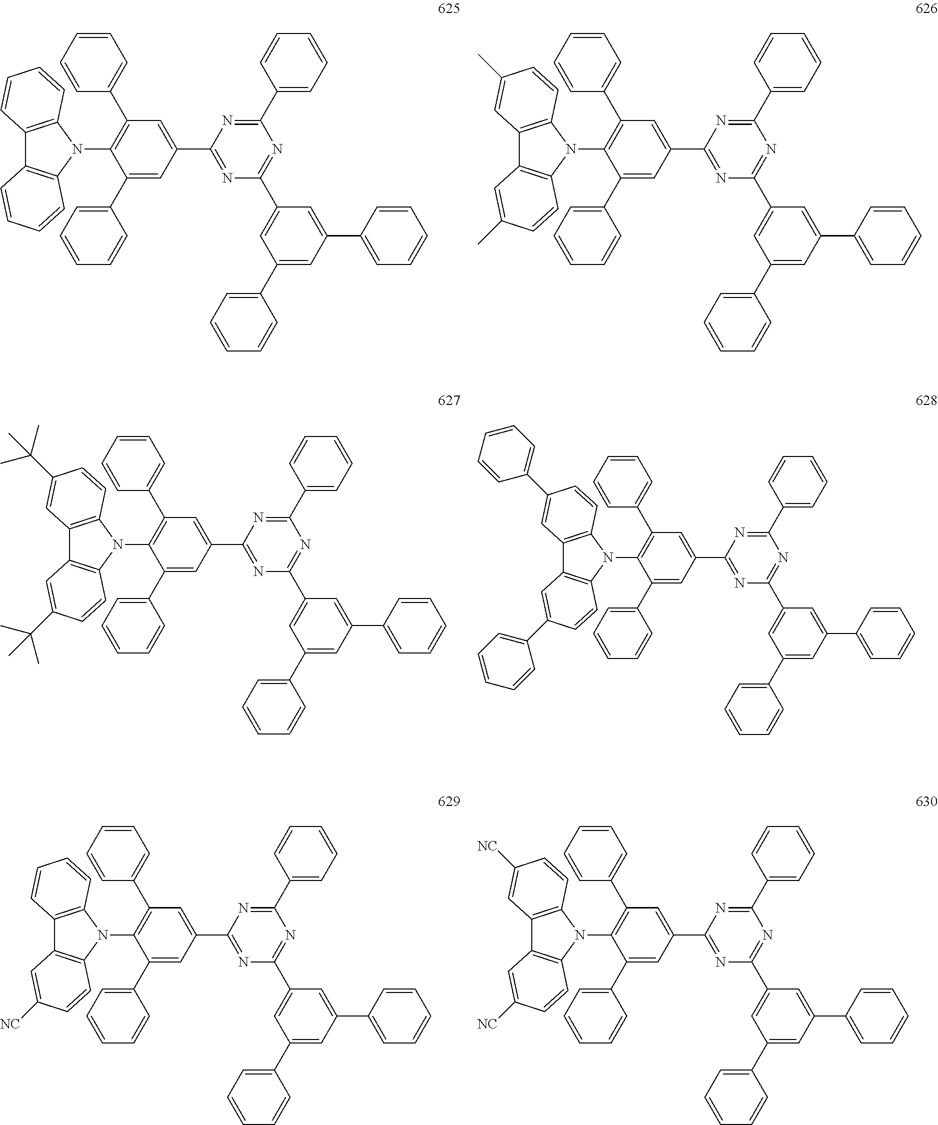

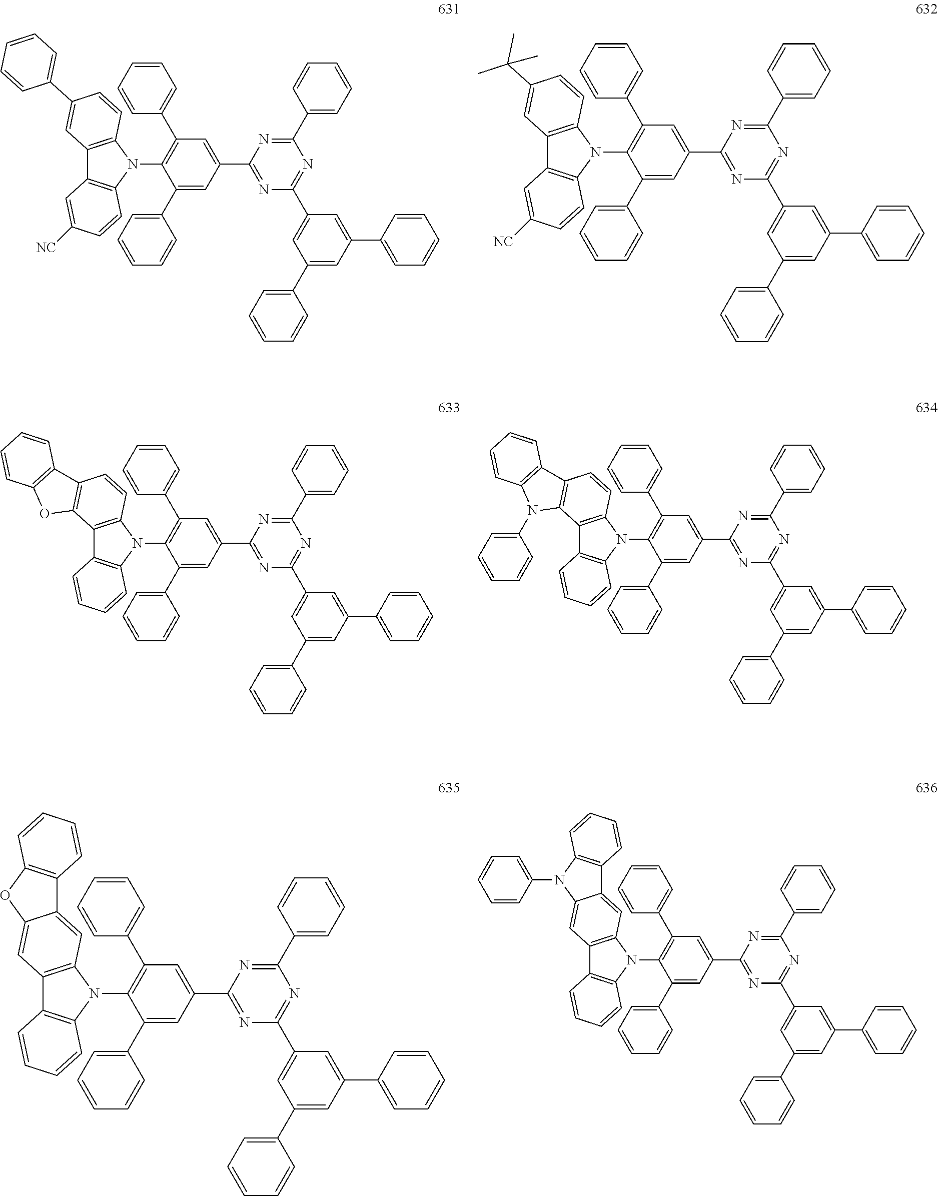

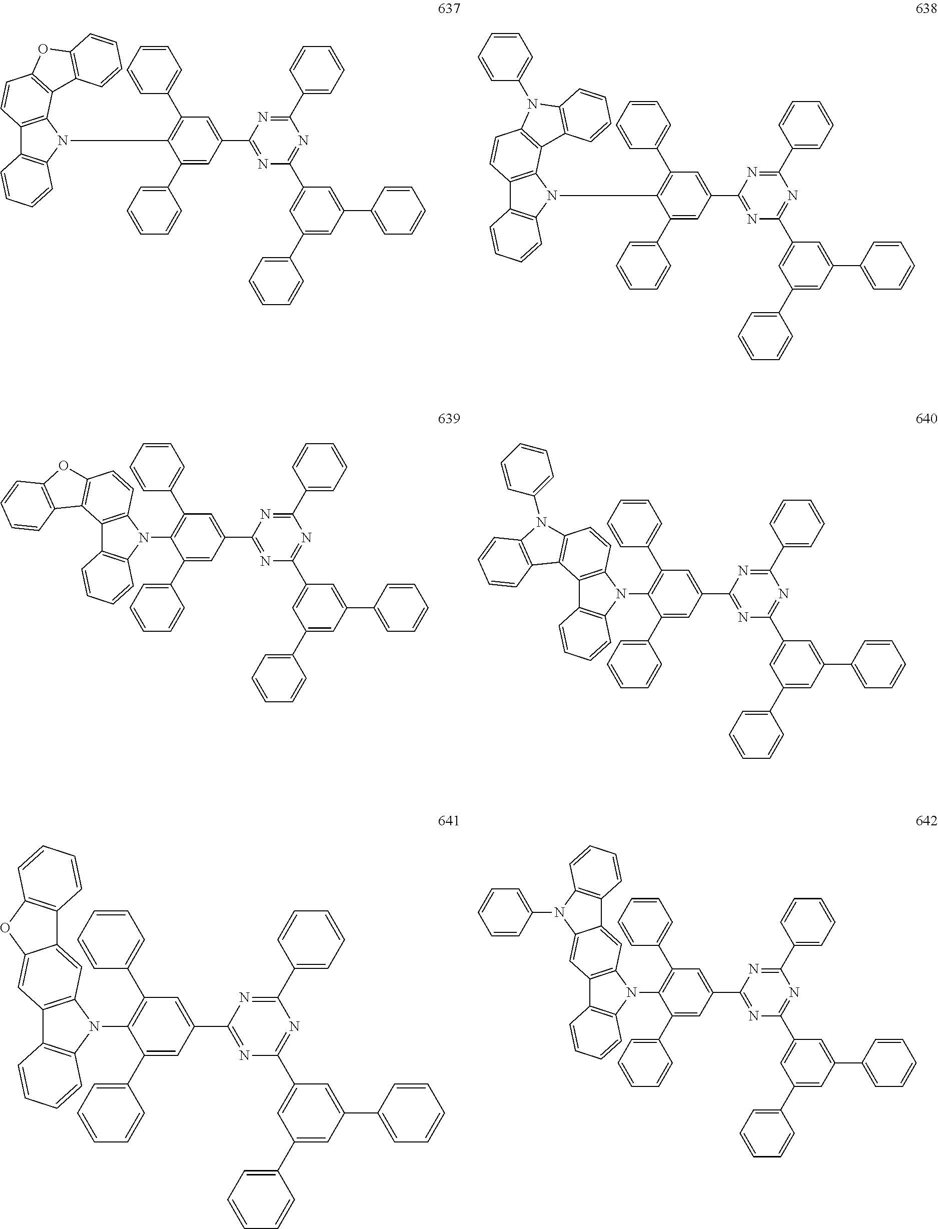

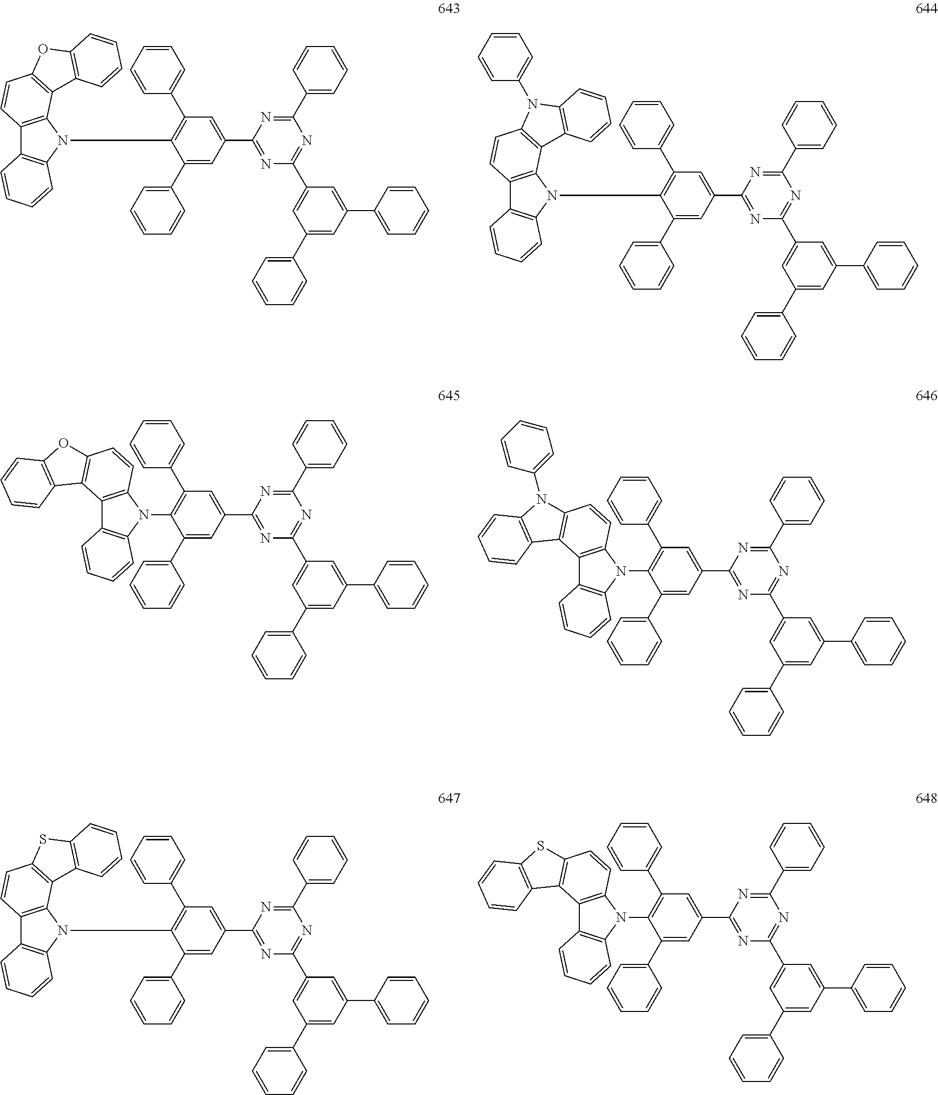

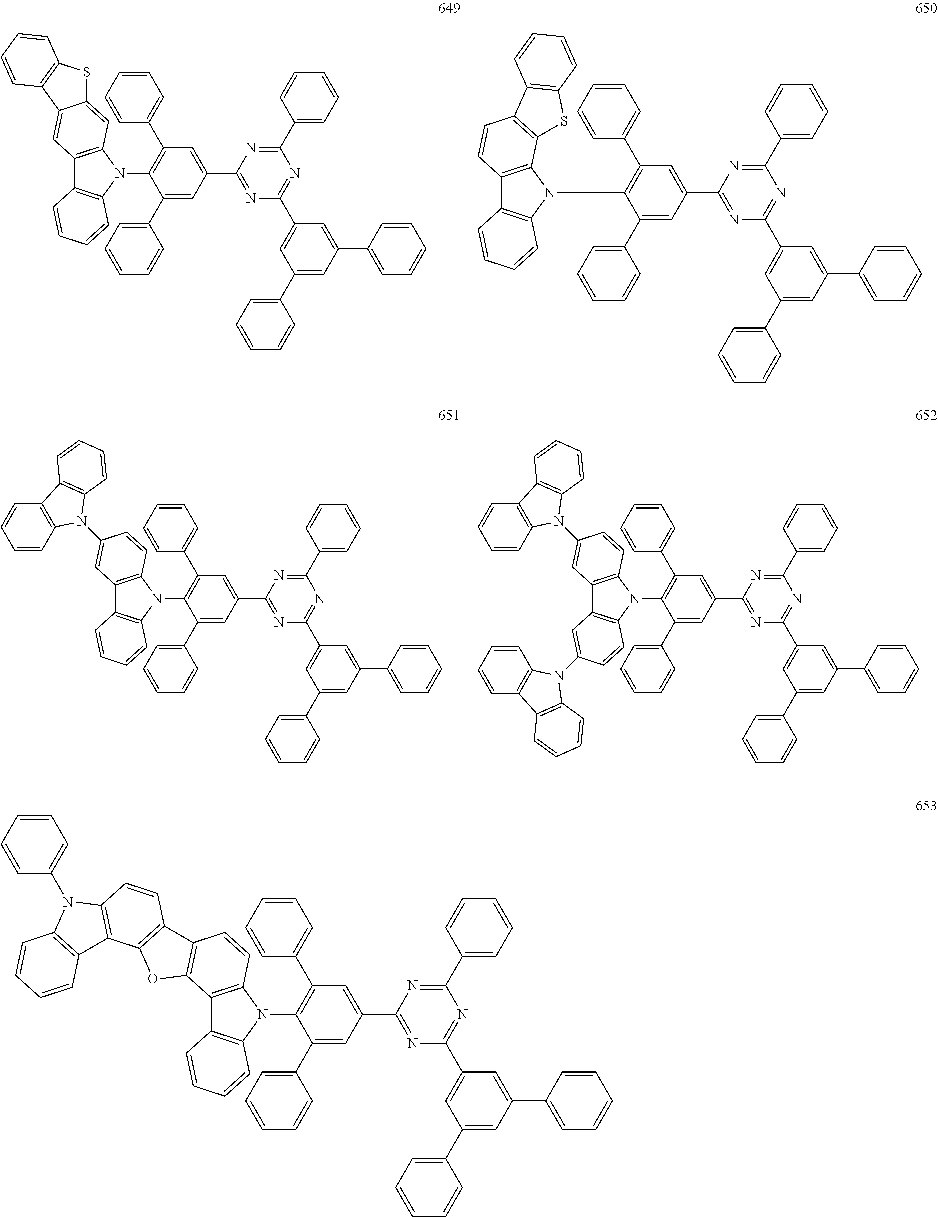

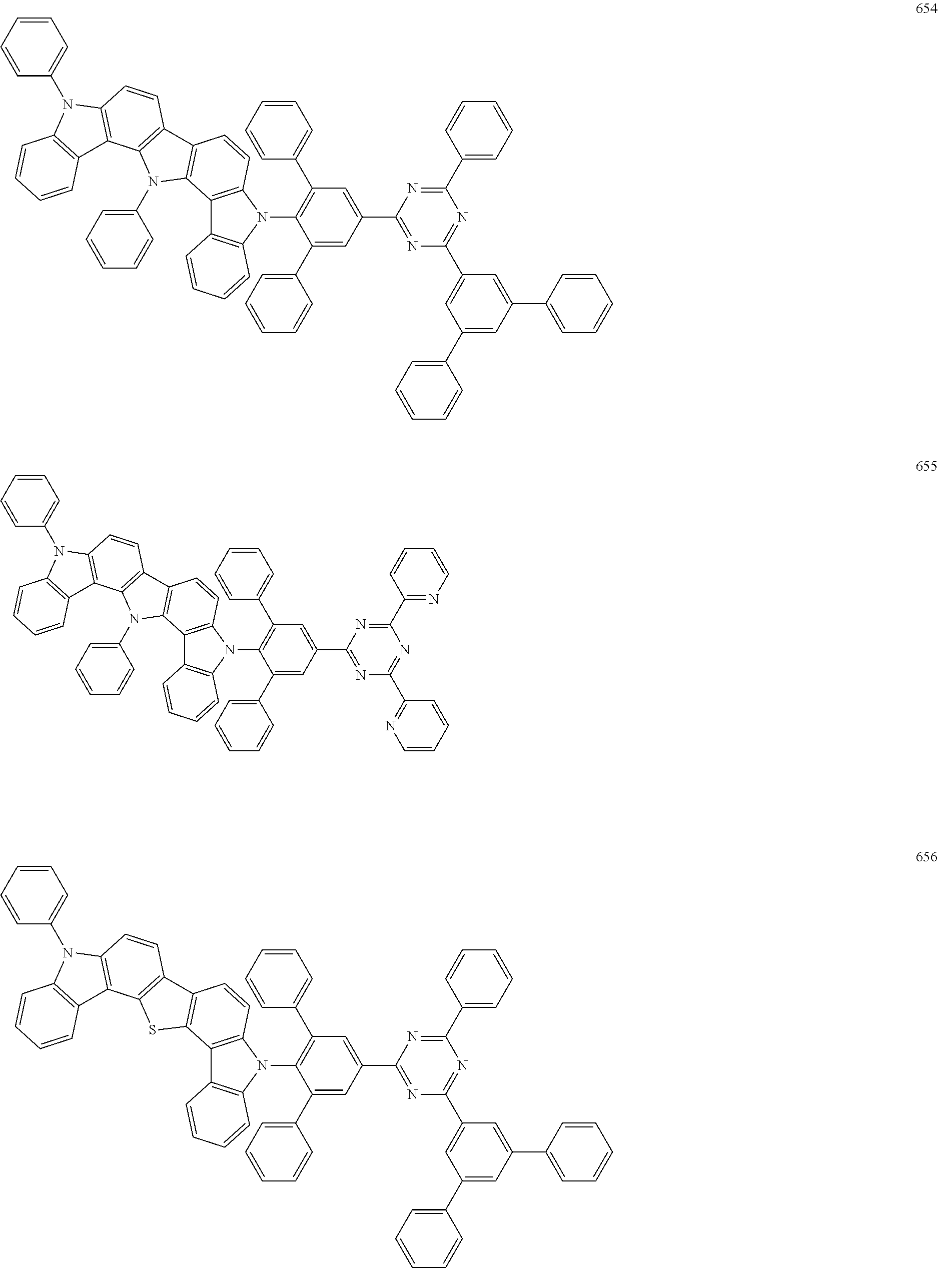 [SMILES: c1ccc(-c2cc(-c3ccccc3)cc(-c3nc(-c4ccccc4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)c2)cc1 ; c1ccc(-c2cc(-c3nc(-c4ccccn4)nc(-c4ccccn4)n3)cc(-c3ccccc3)c2-n2c3ccccc3c3c2ccc2c4ccc5c(c6ccccc6n5-c5ccccc5)c4n(-c4ccccc4)c23)cc1 ; c1ccc(-c2cc(-c3ccccc3)cc(-c3nc(-c4ccccc4)nc(-c4cc(-c5ccccc5)c(-n5c6ccccc6c6c7sc8c(ccc9c8c8ccccc8n9-c8ccccc8)c7ccc65)c(-c5ccccc5)c4)n3)c2)cc1]

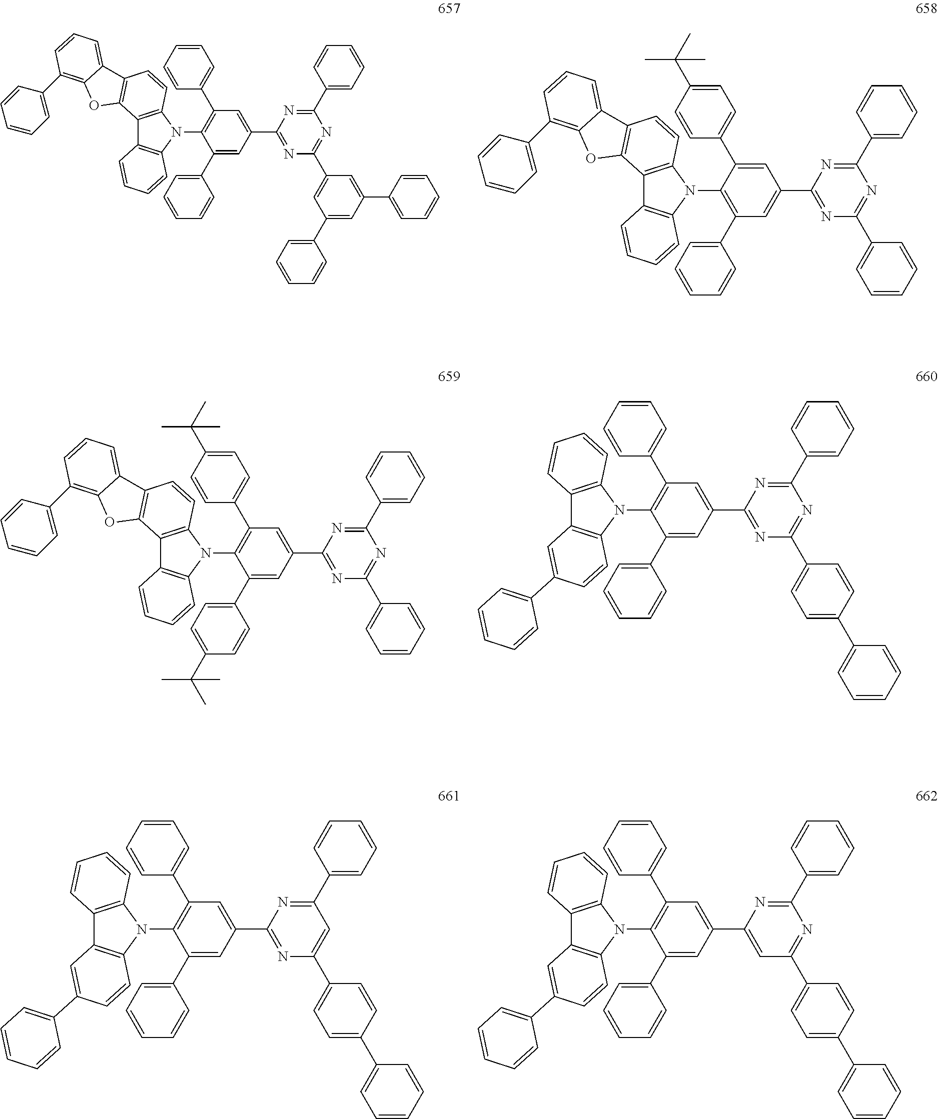

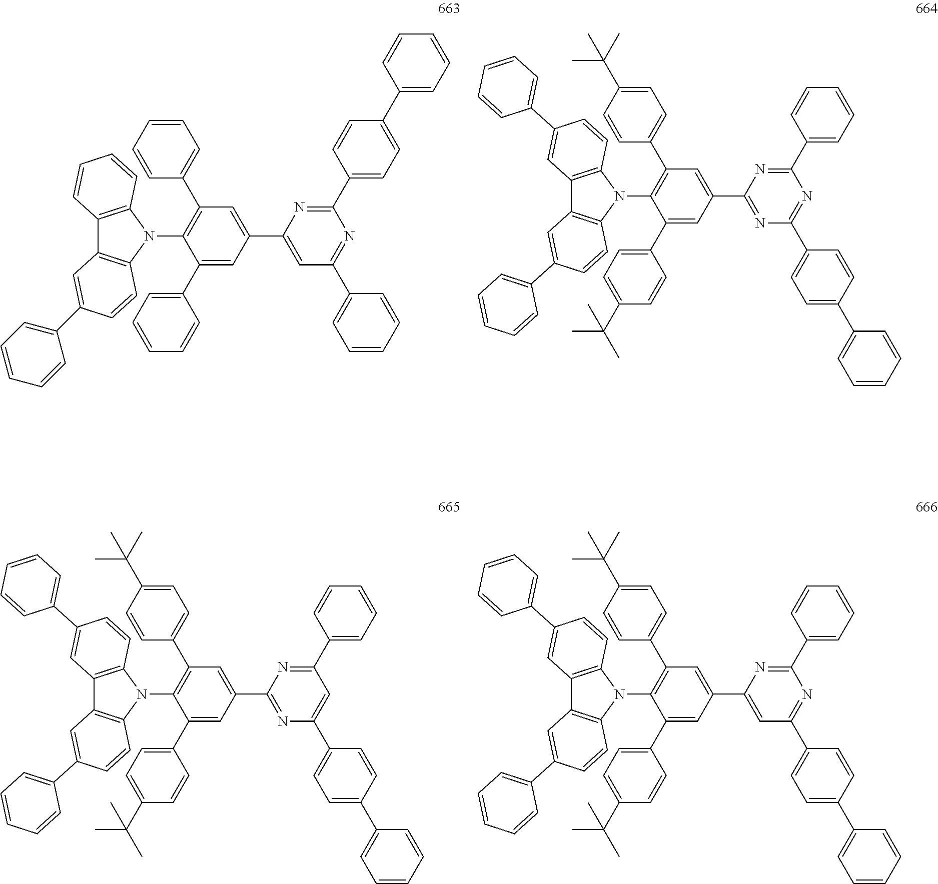

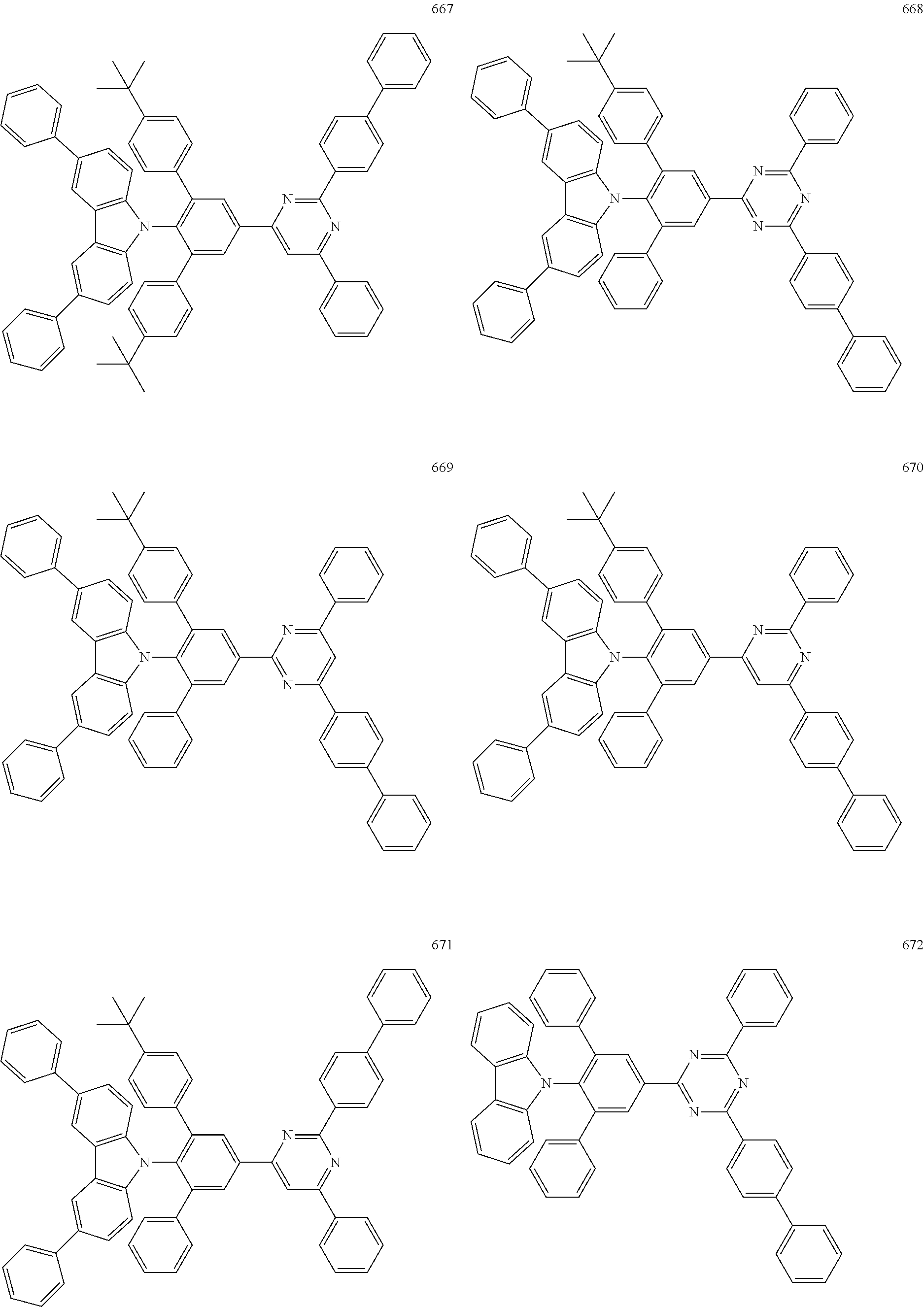

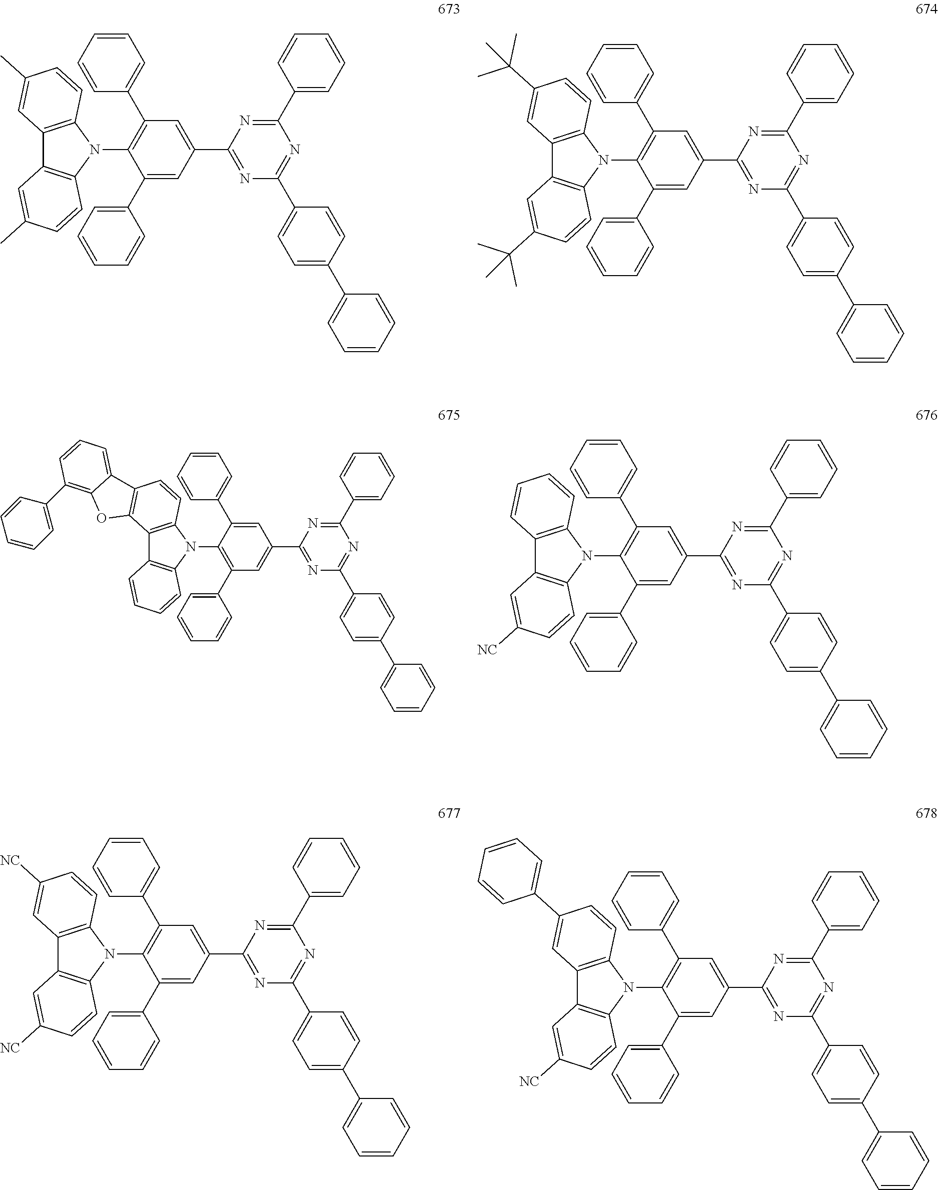

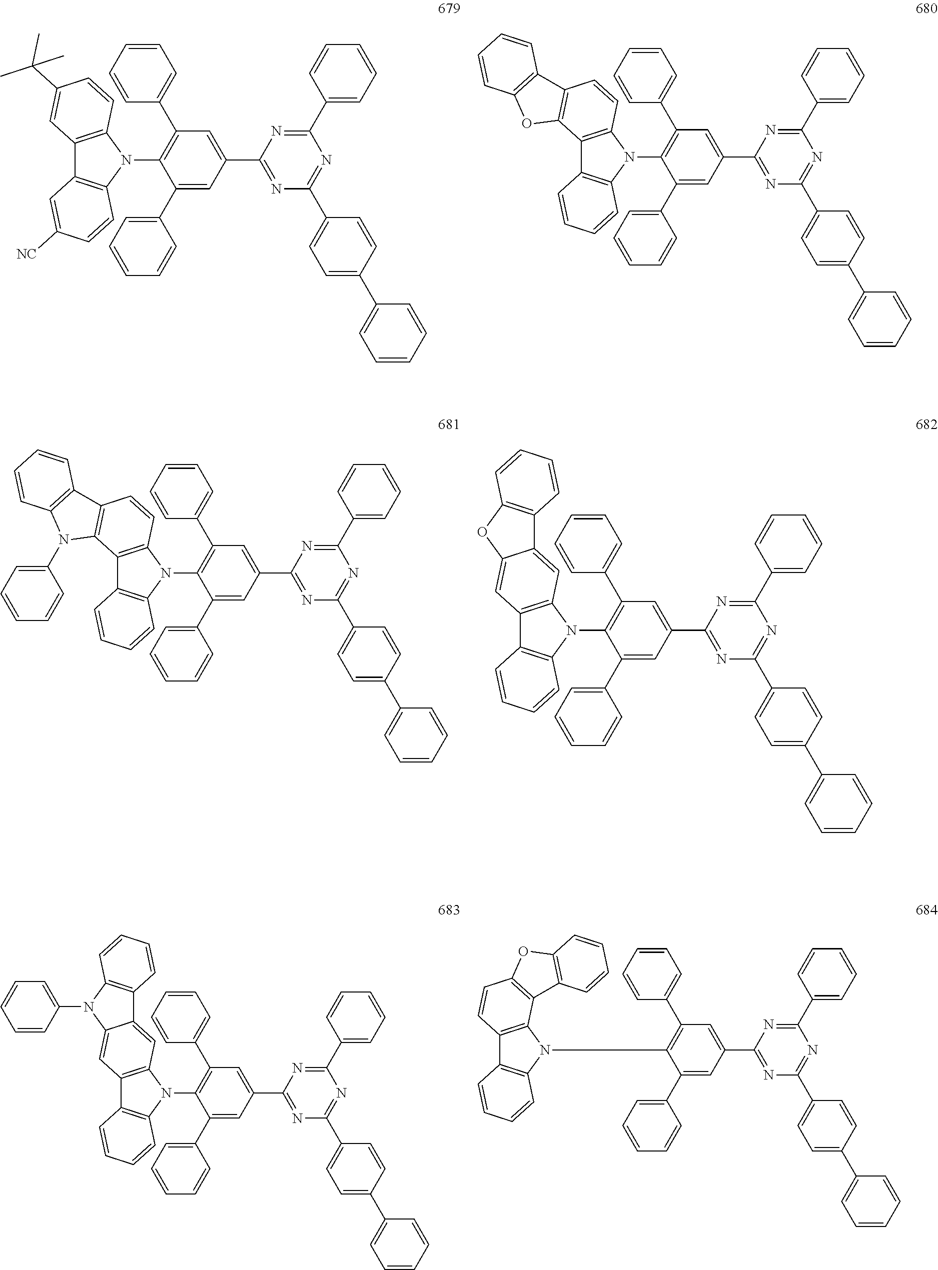

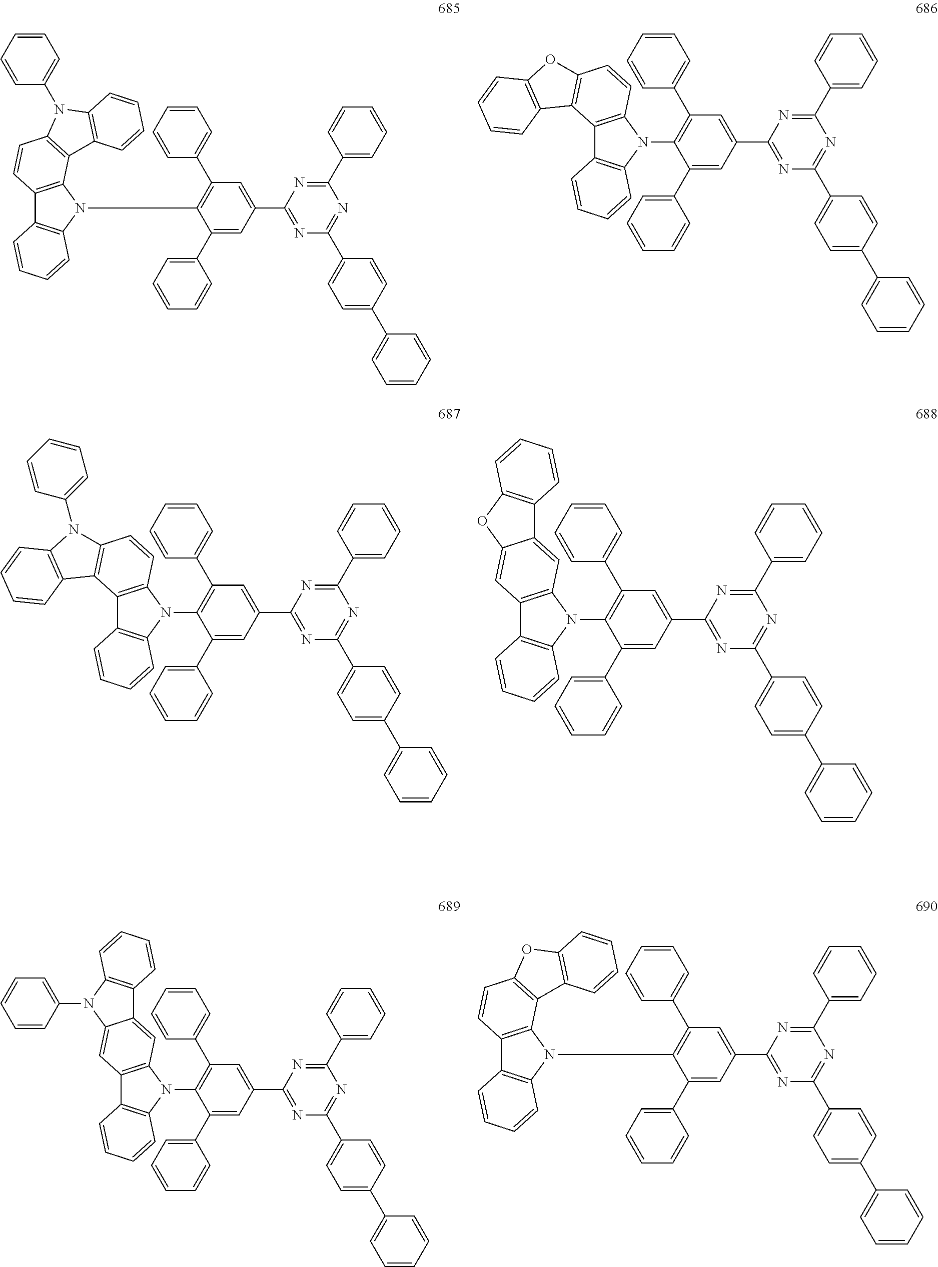

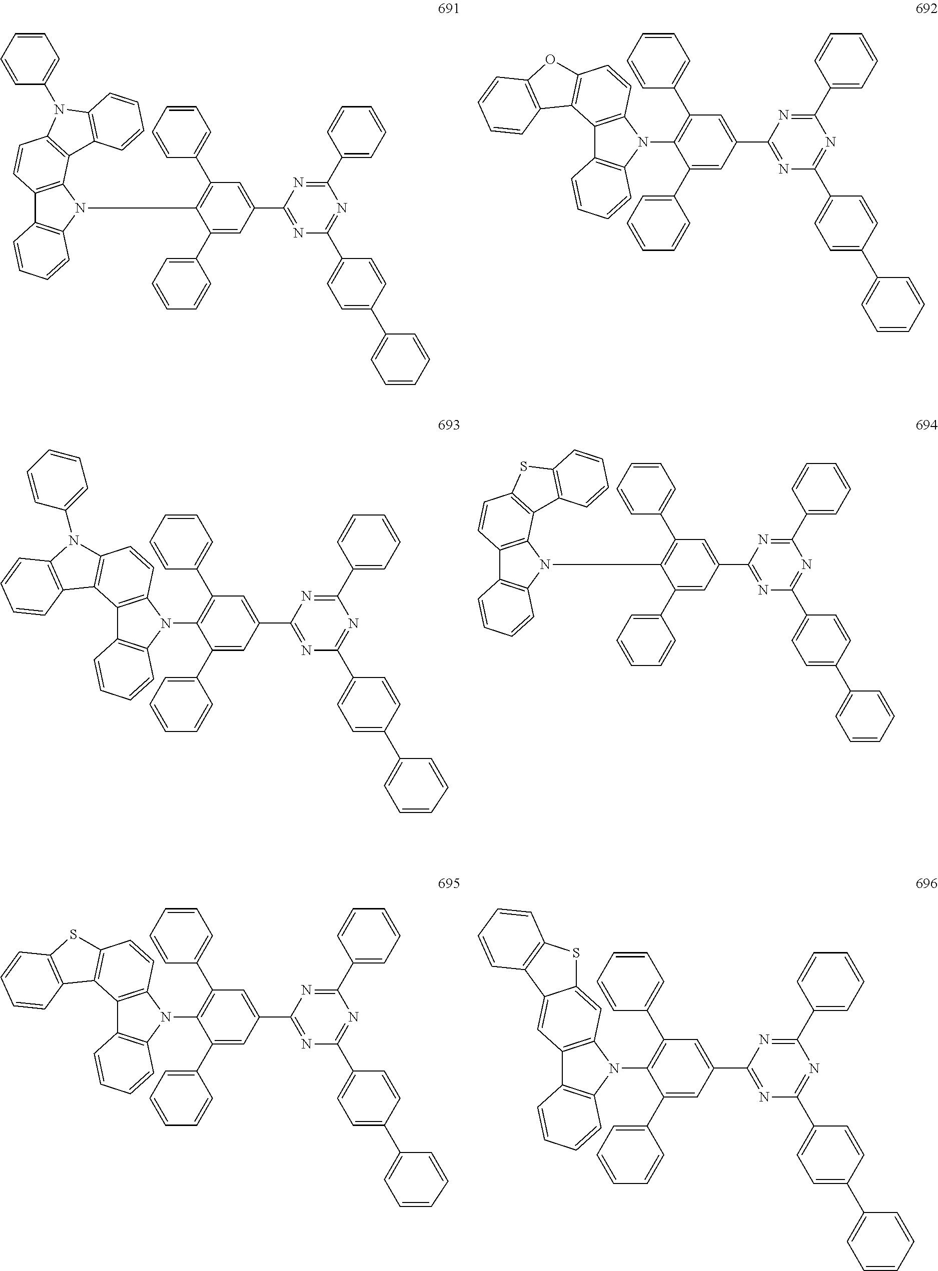

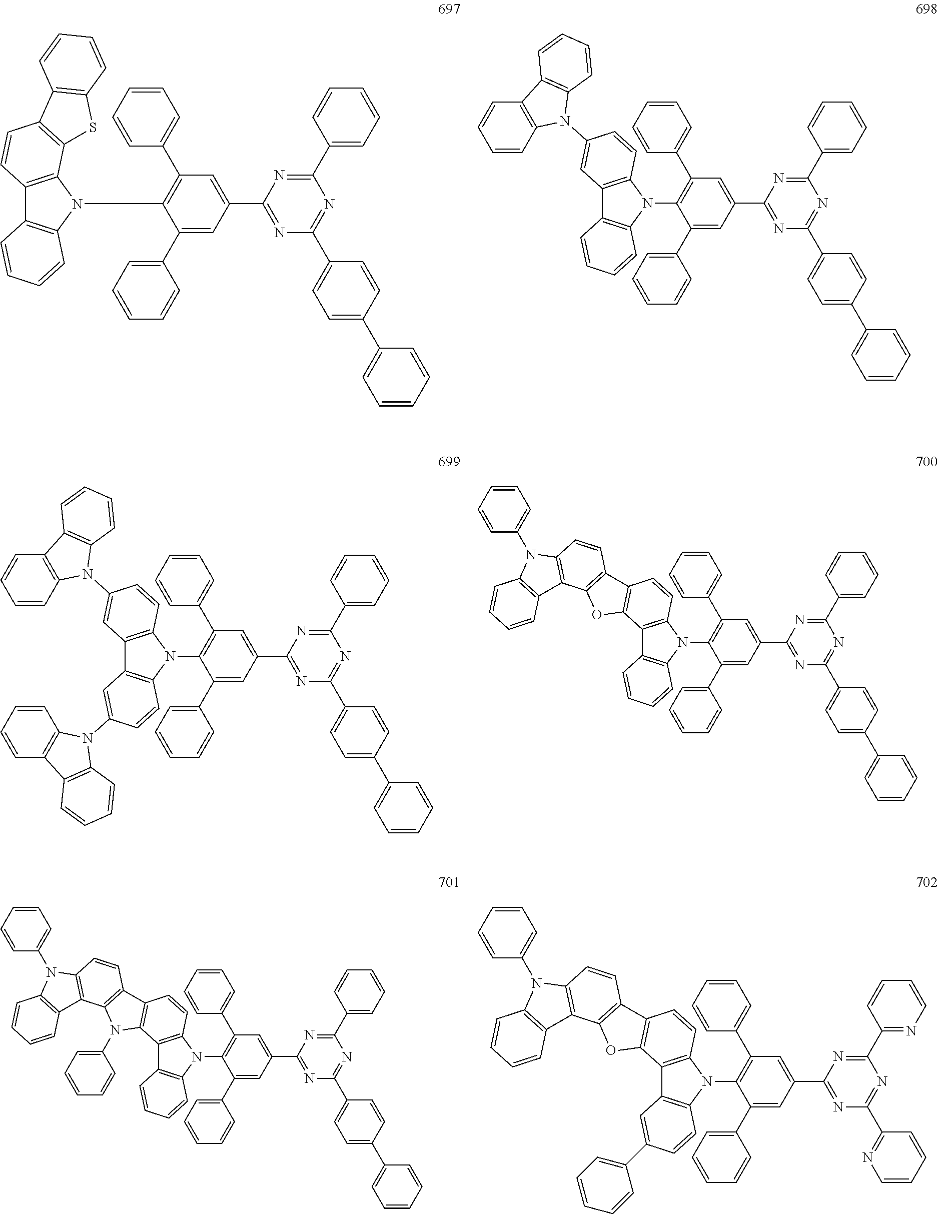

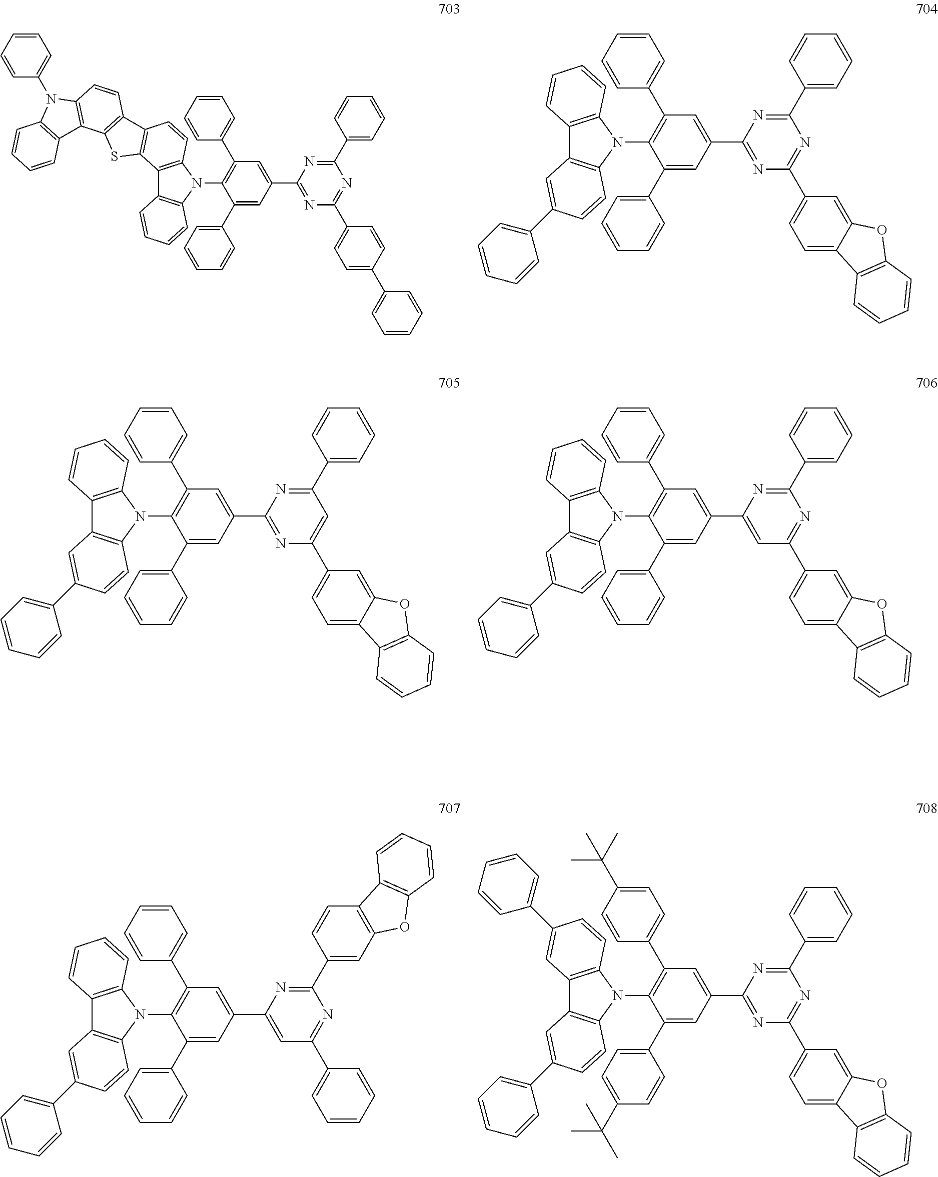

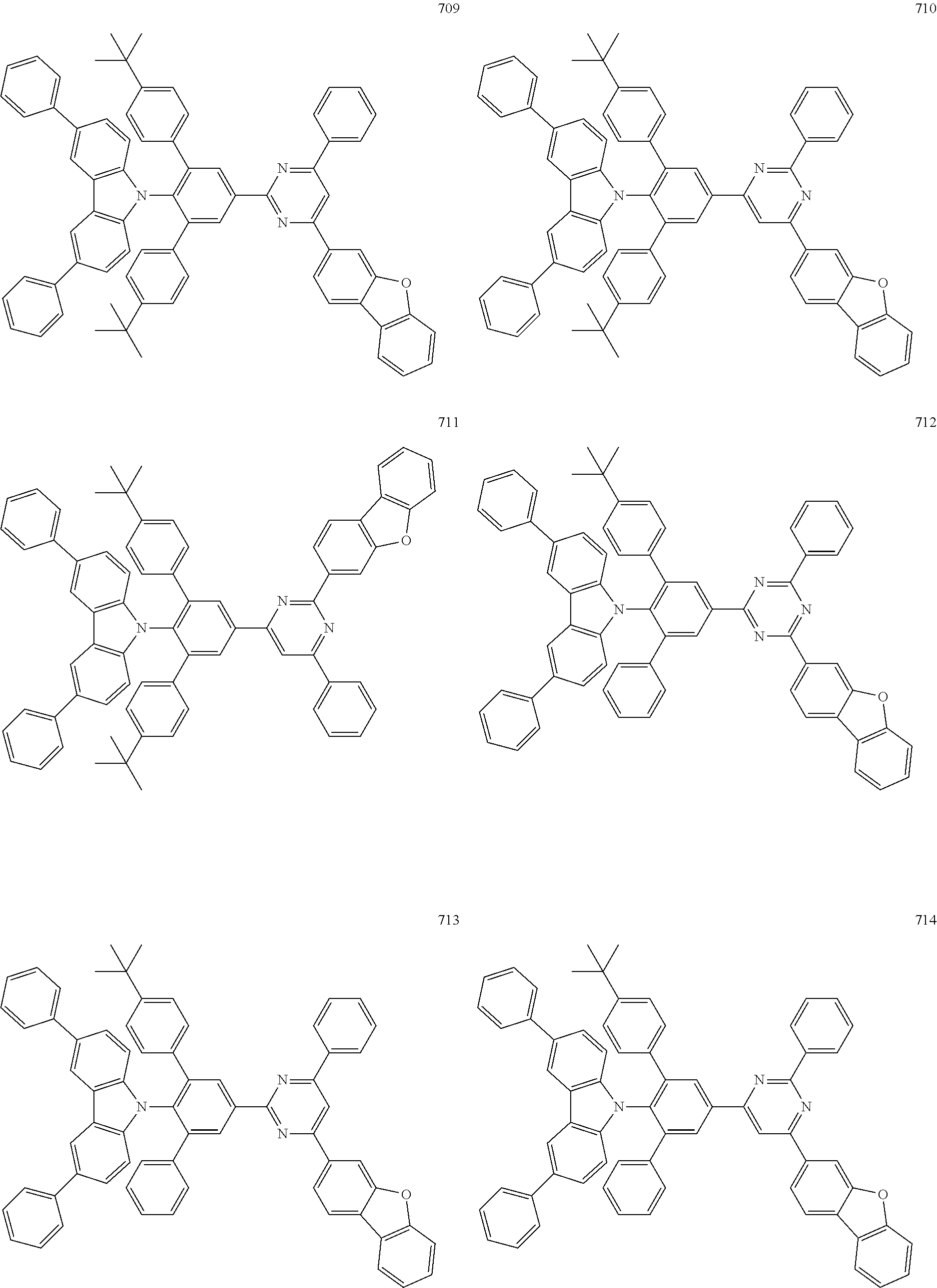

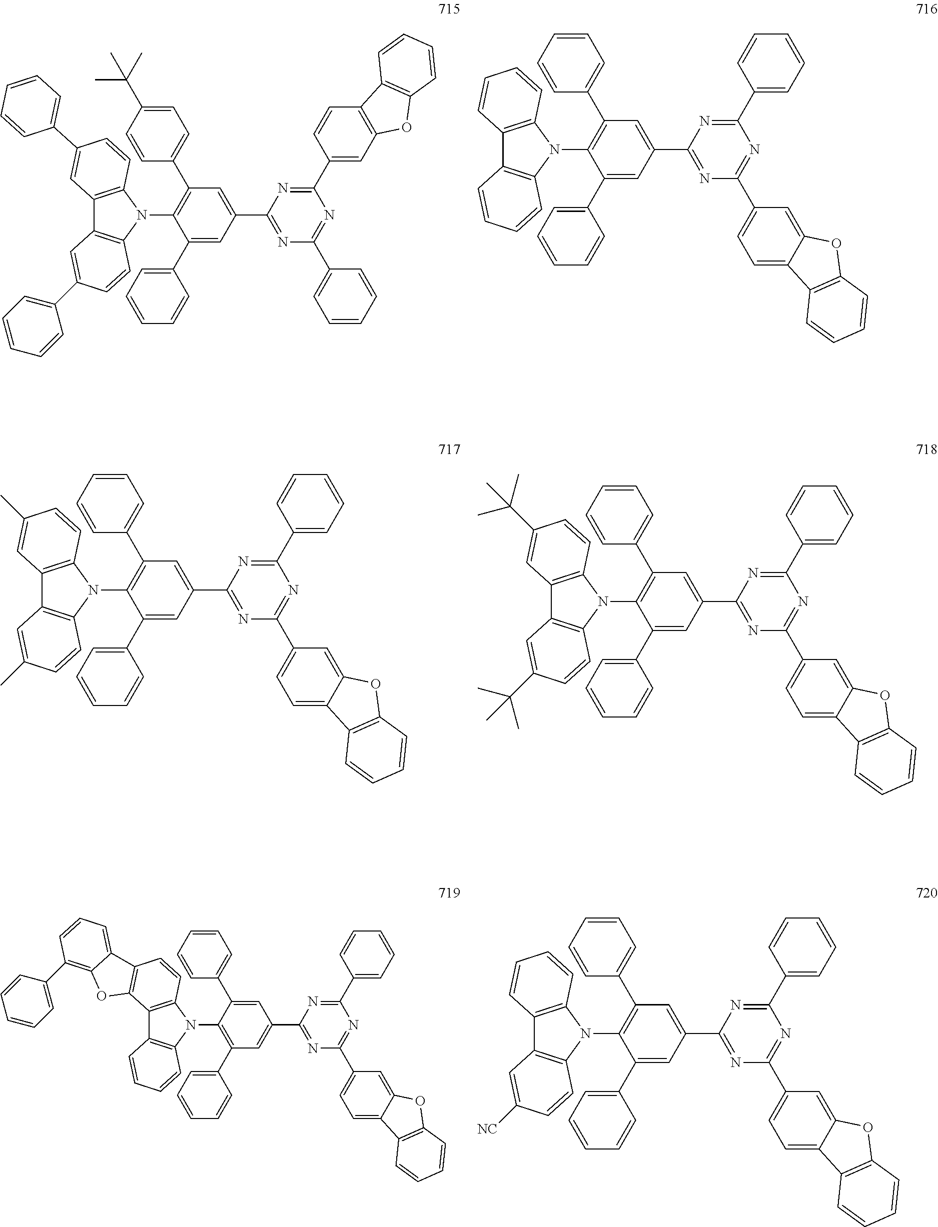

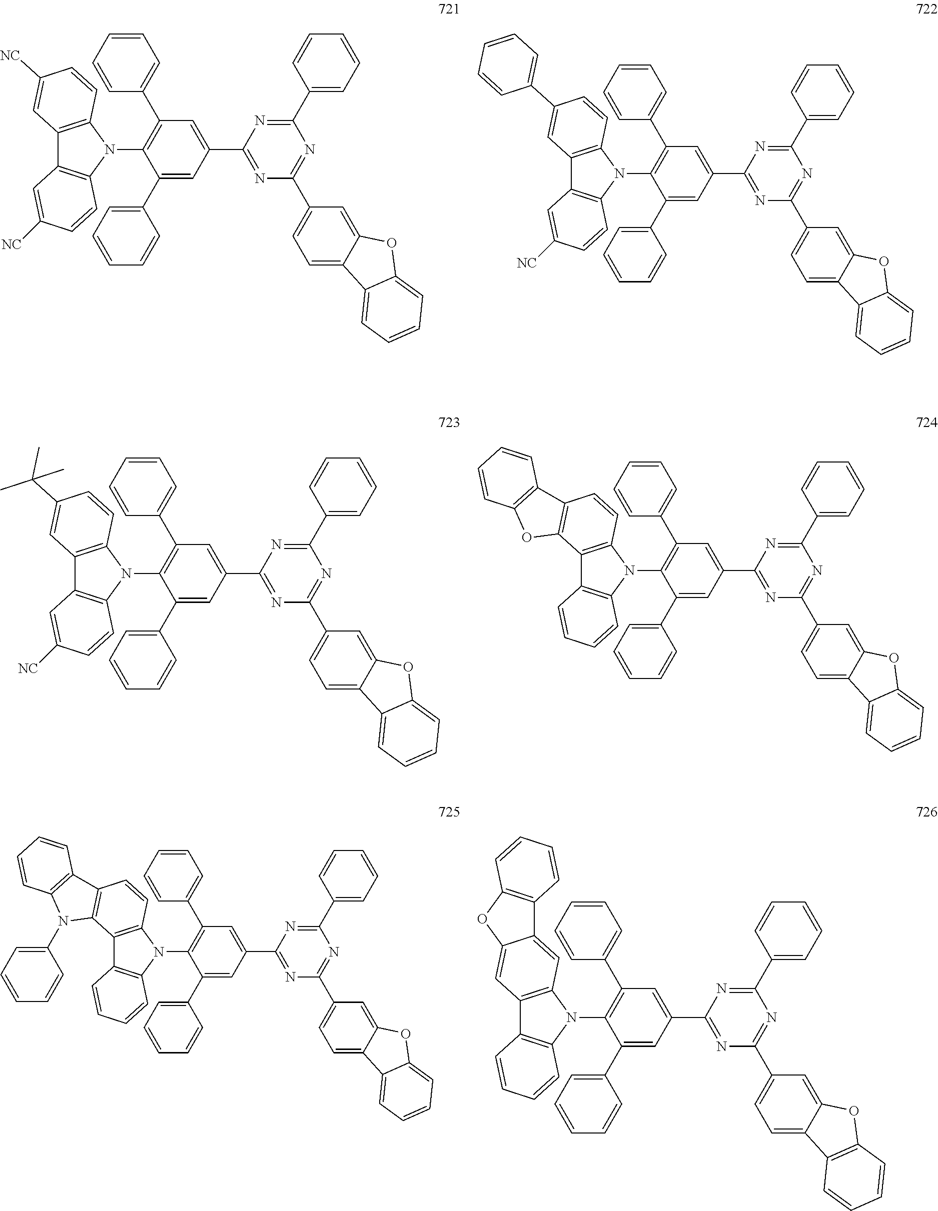

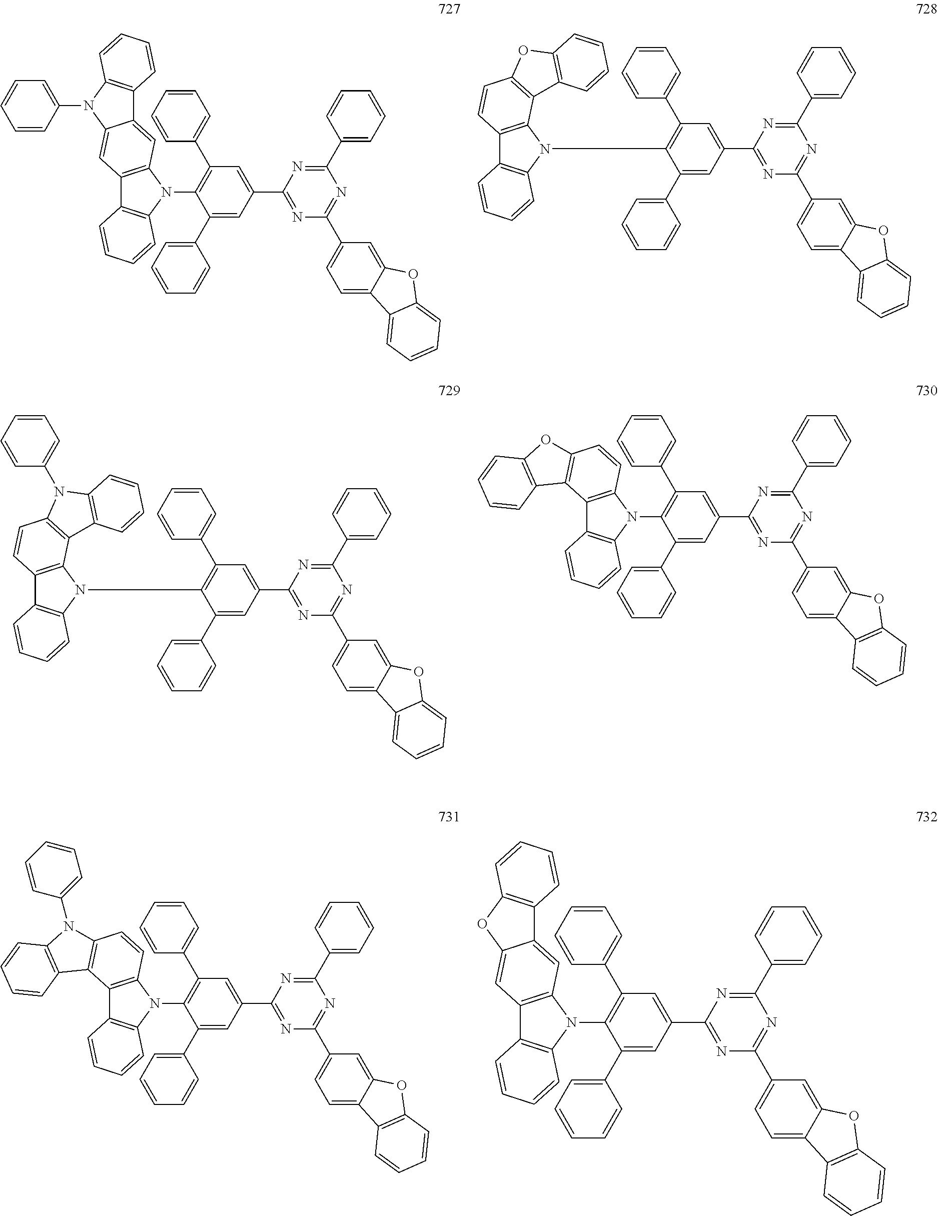

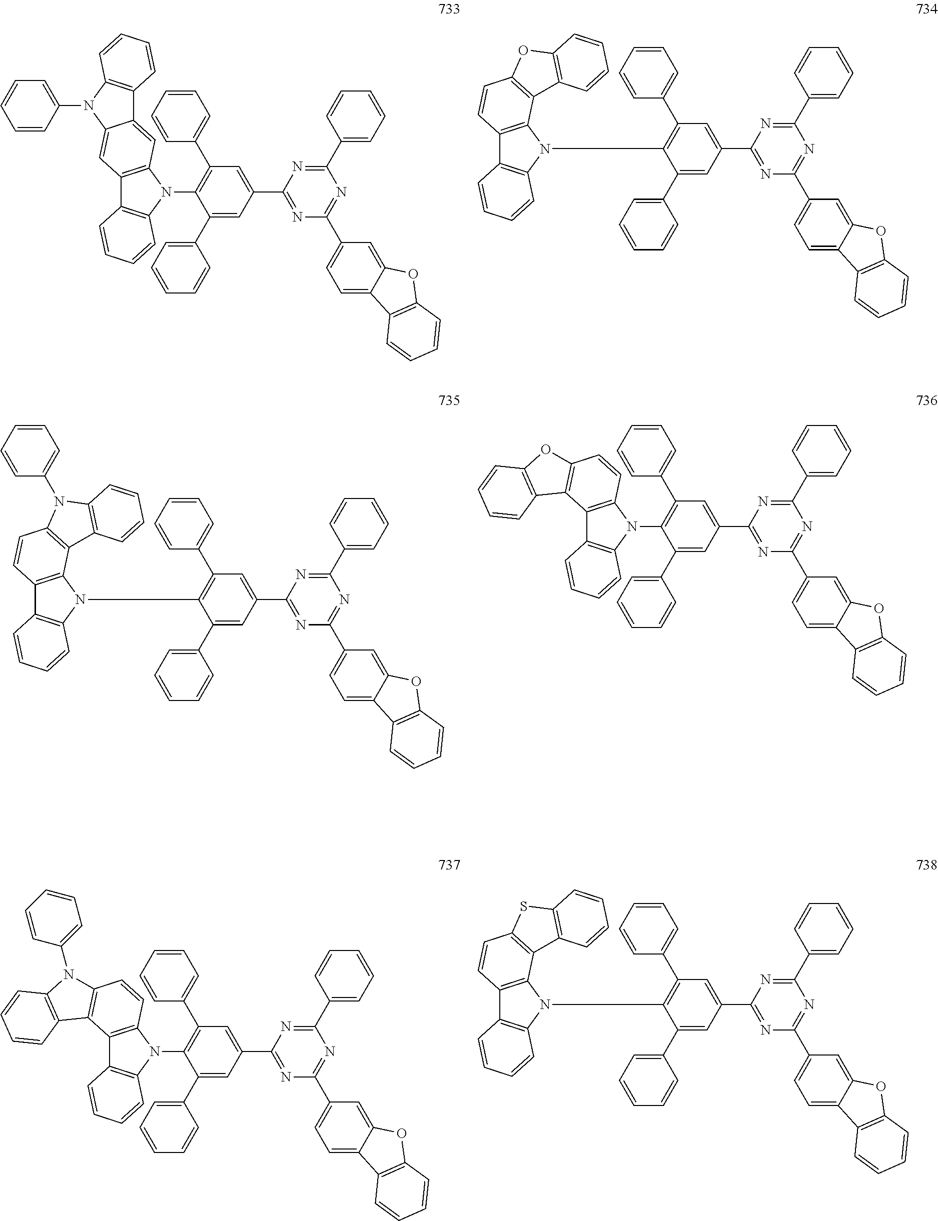

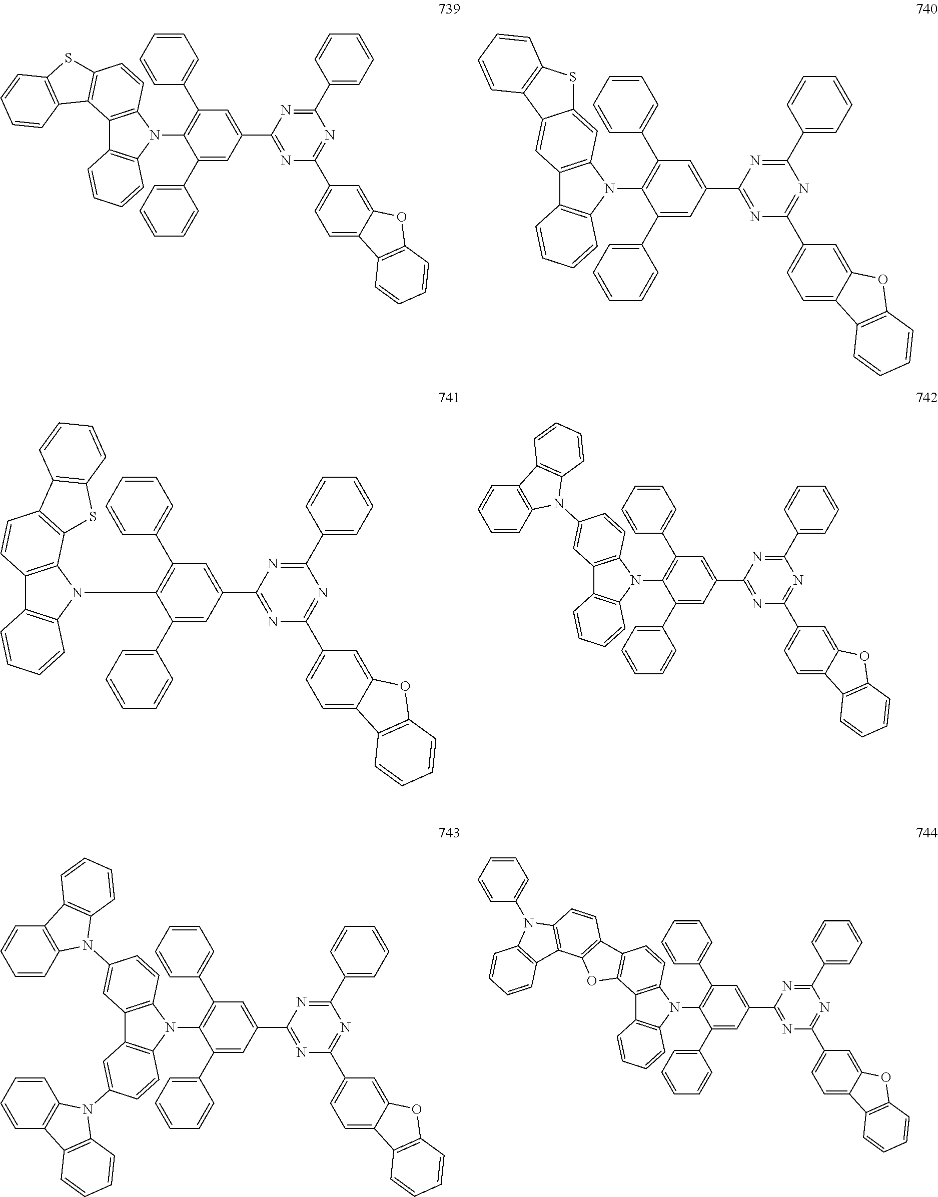

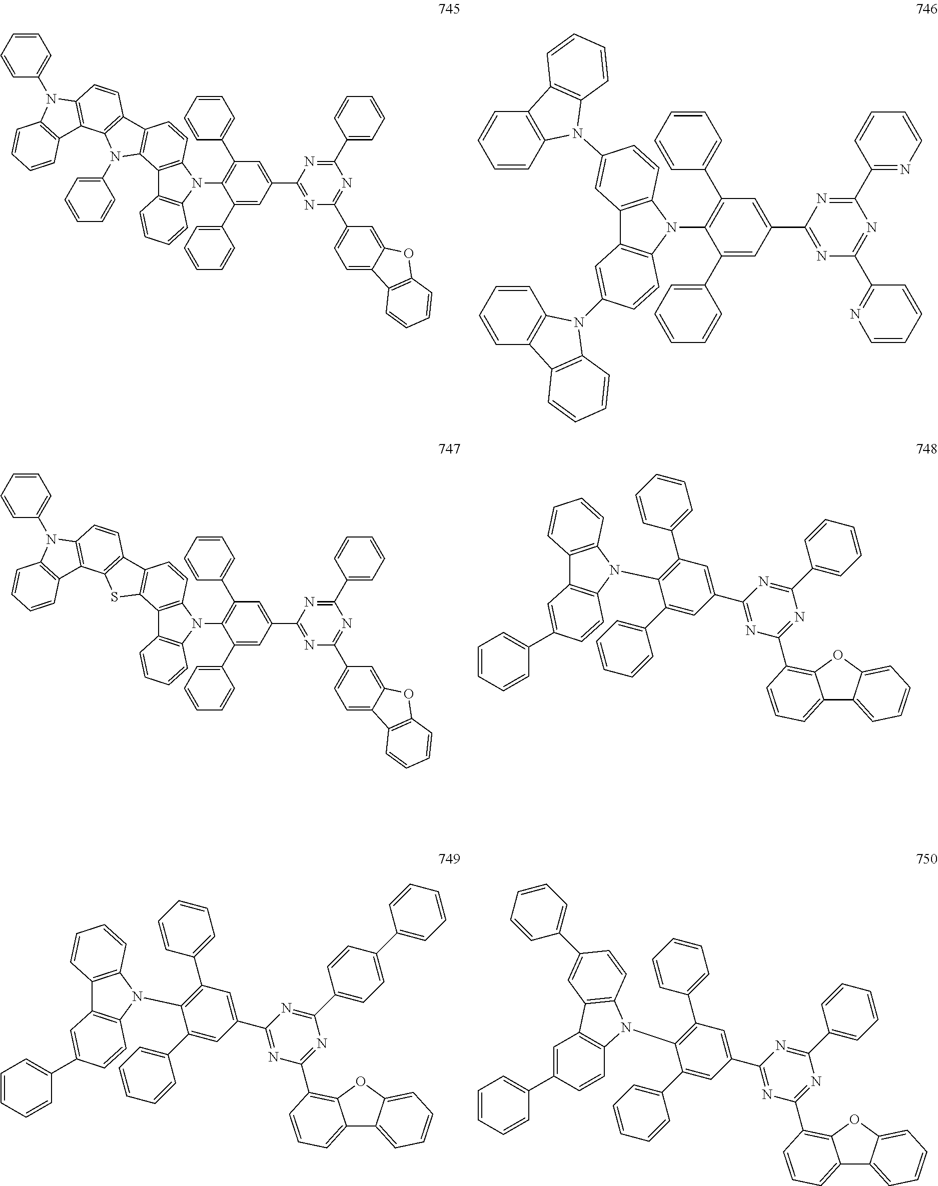

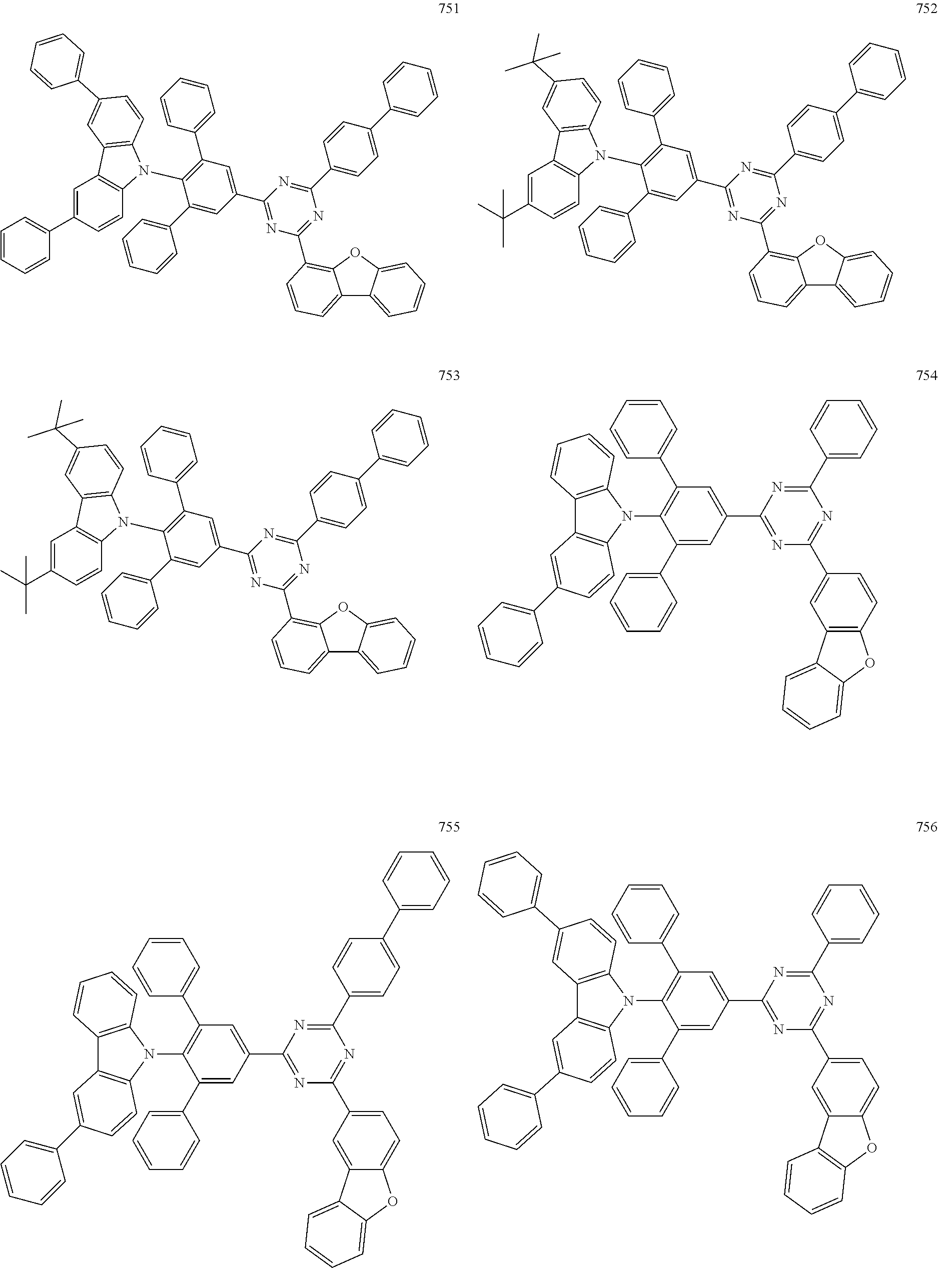

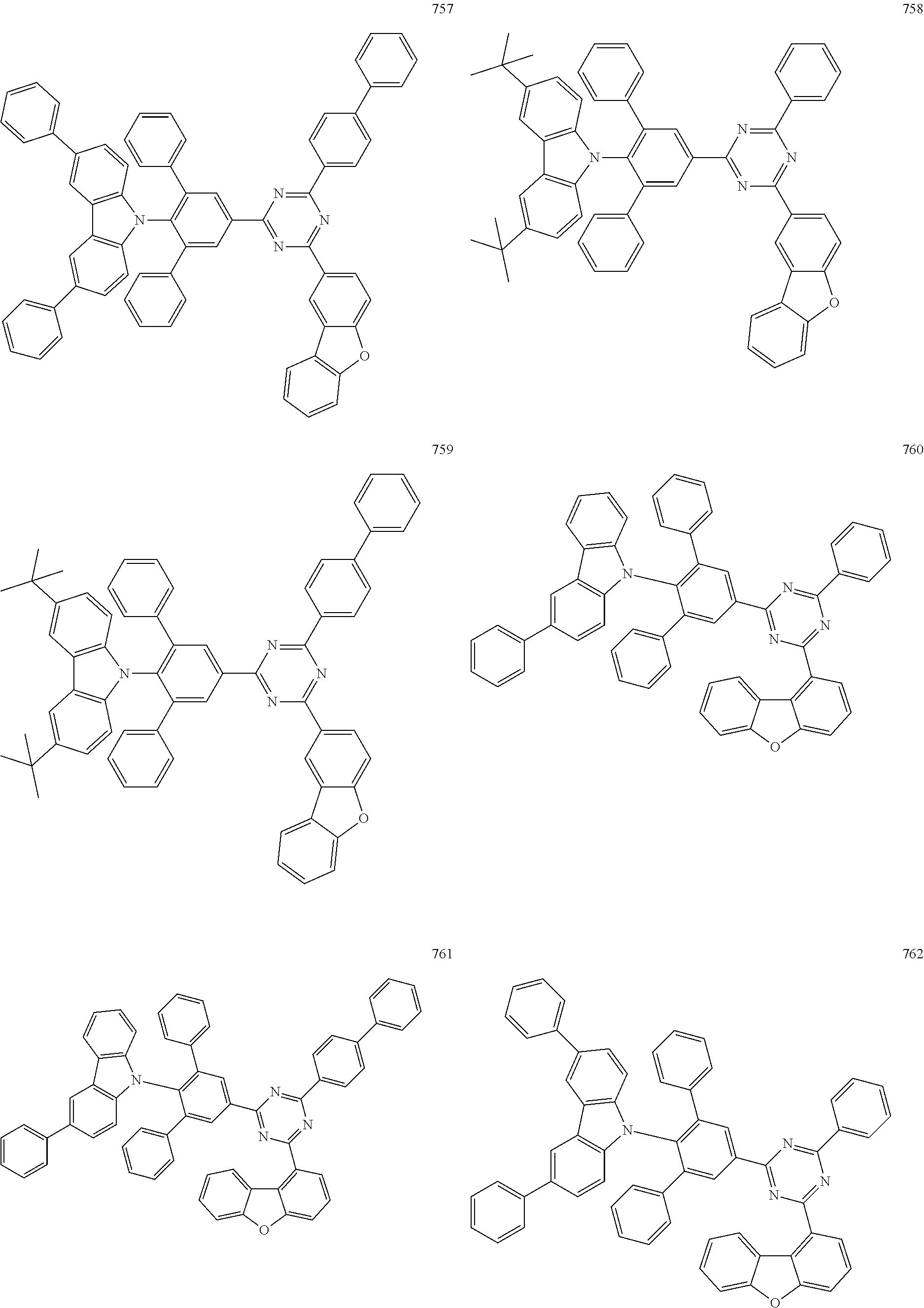

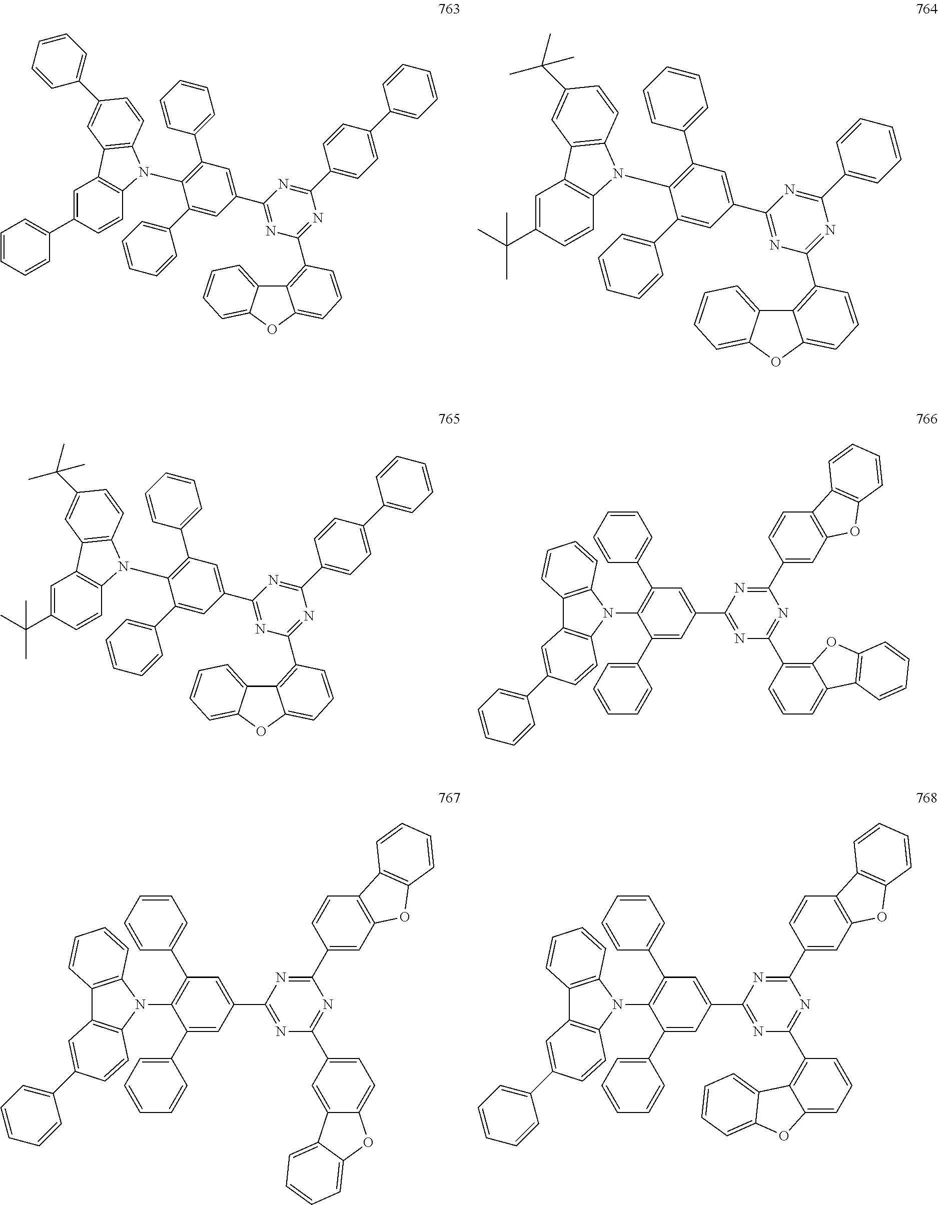

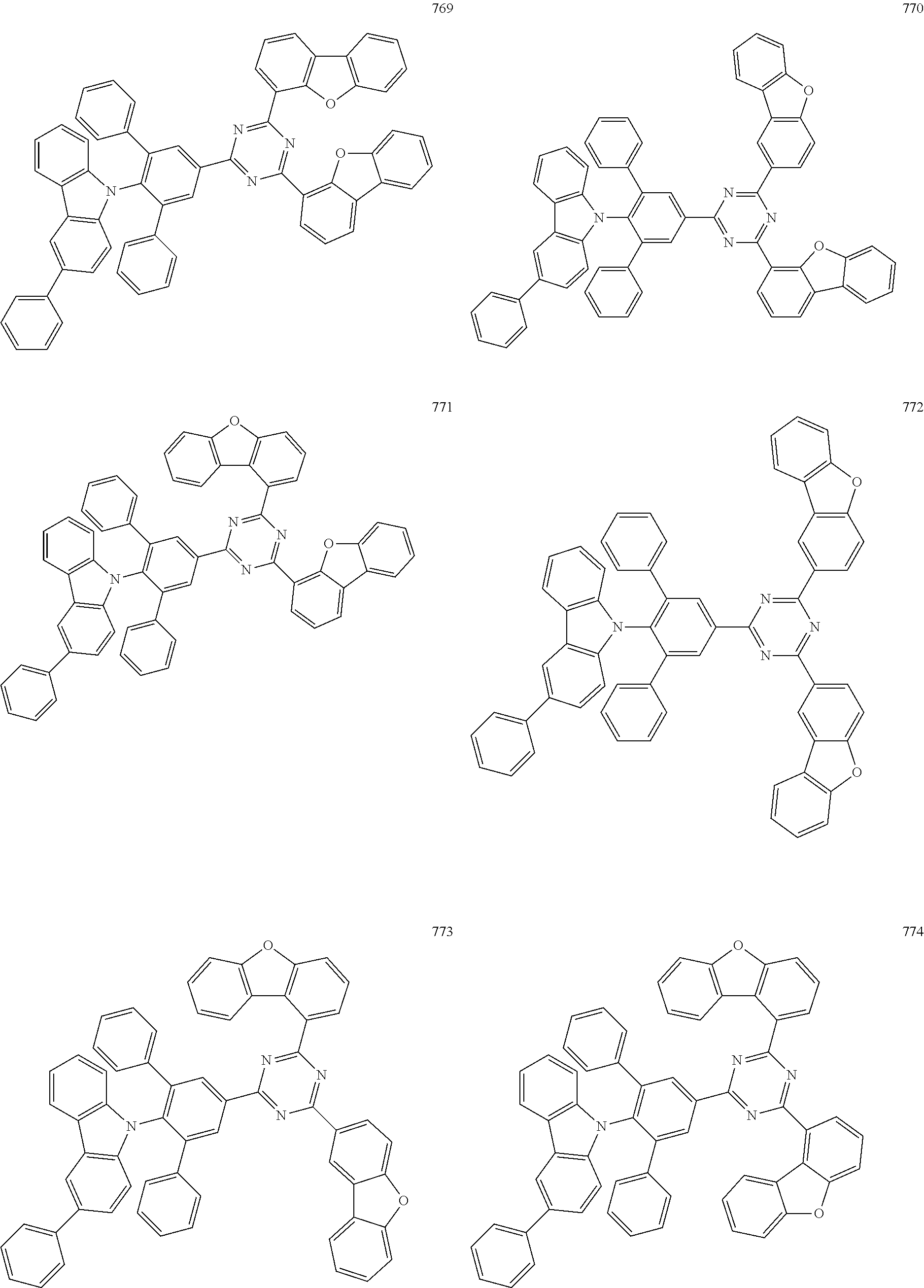

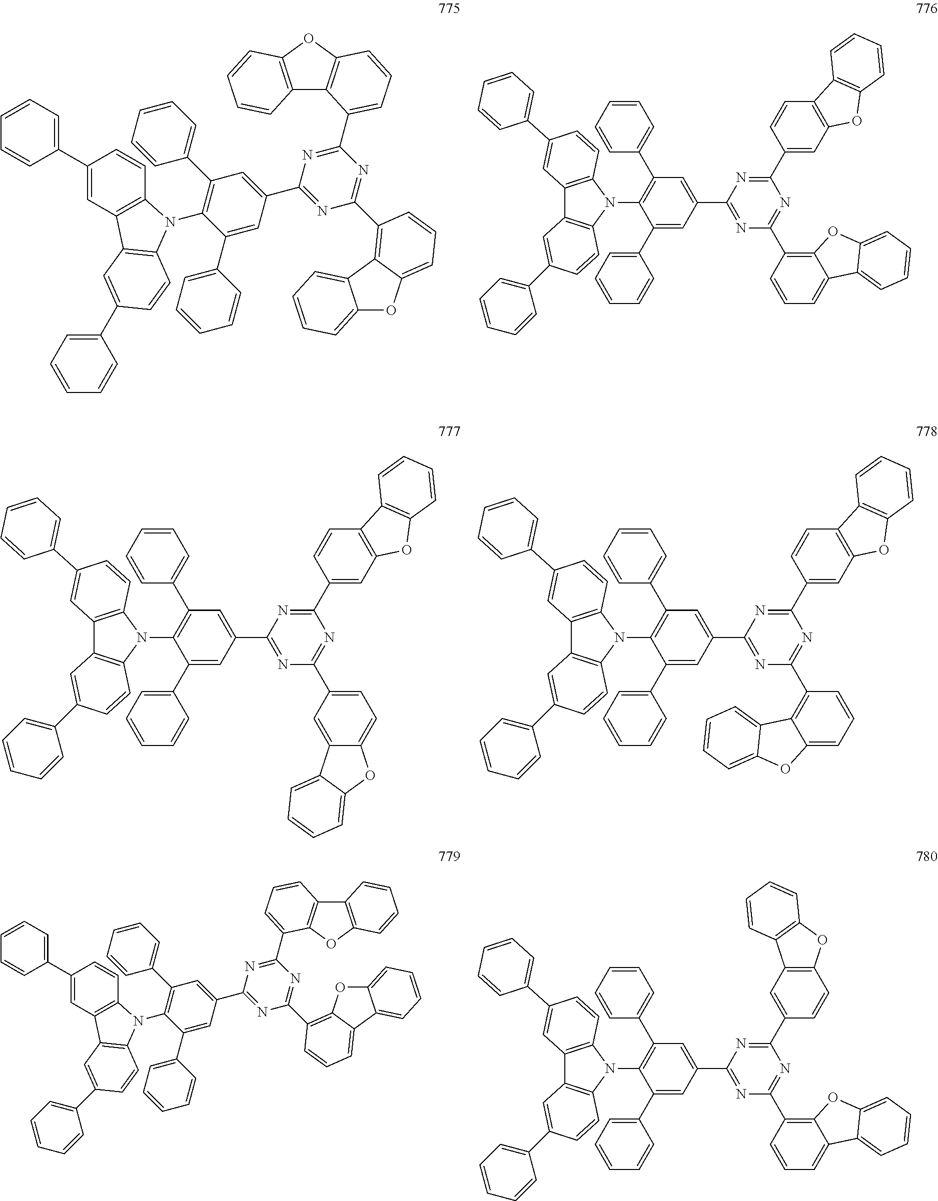

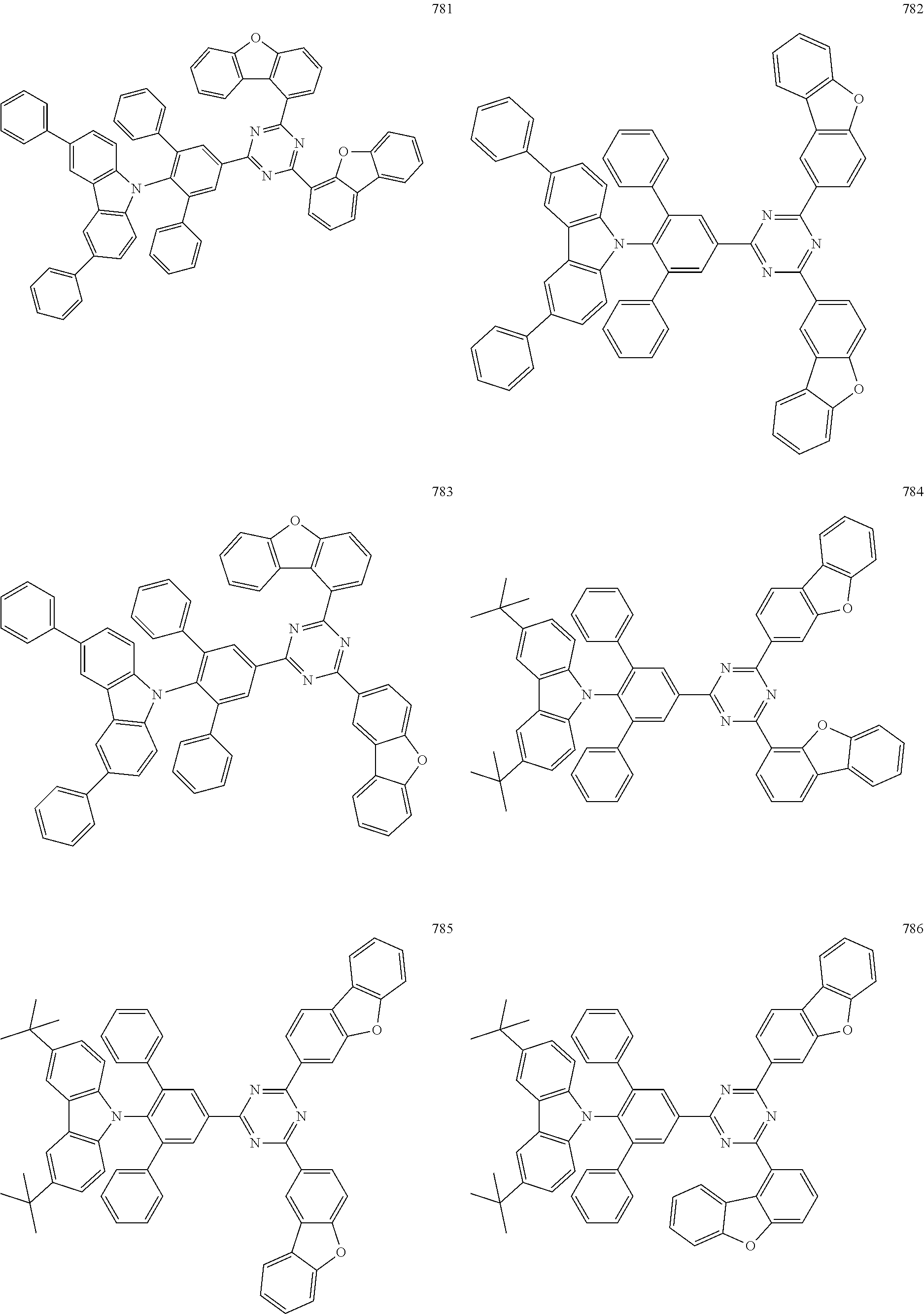

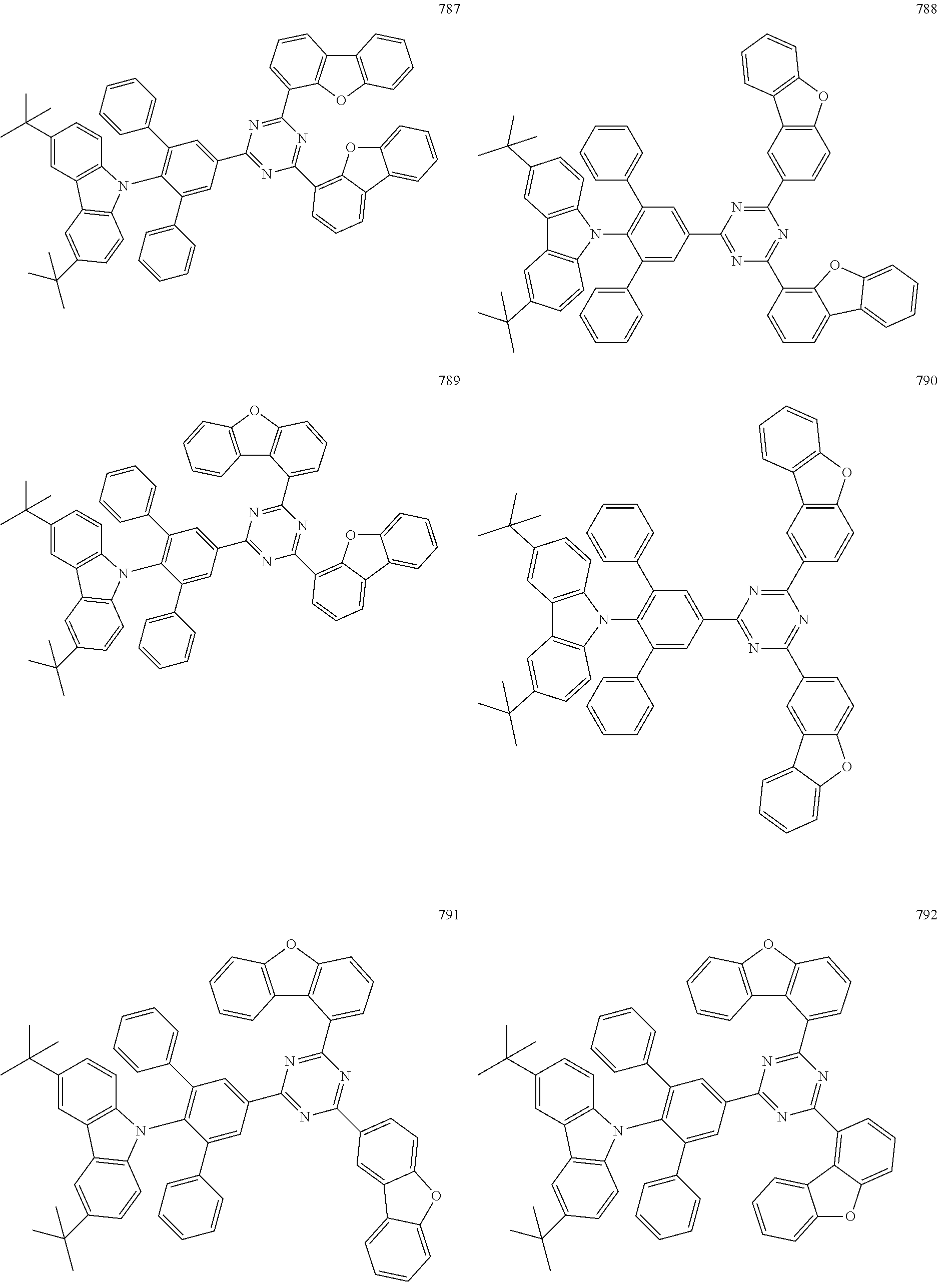

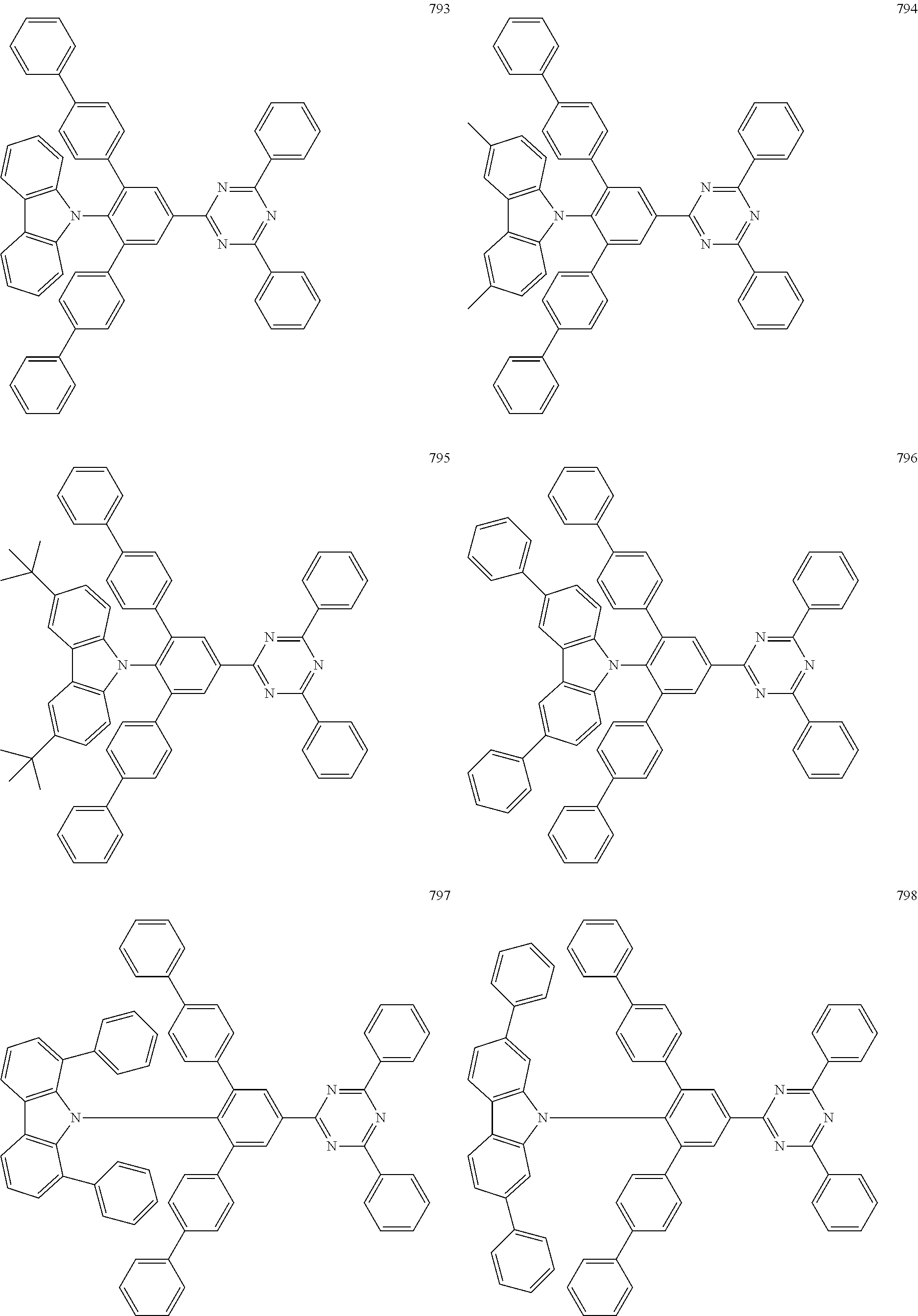

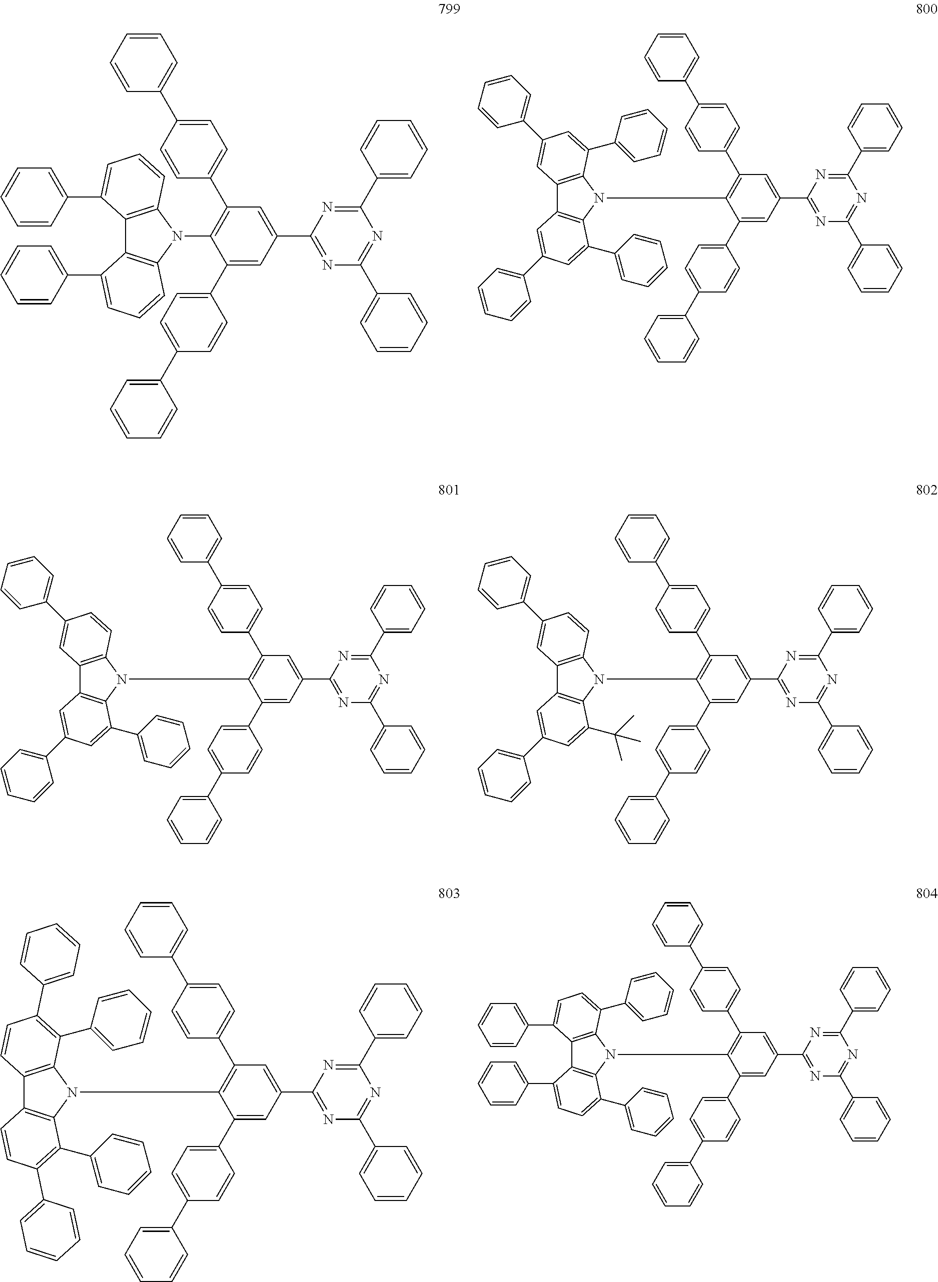

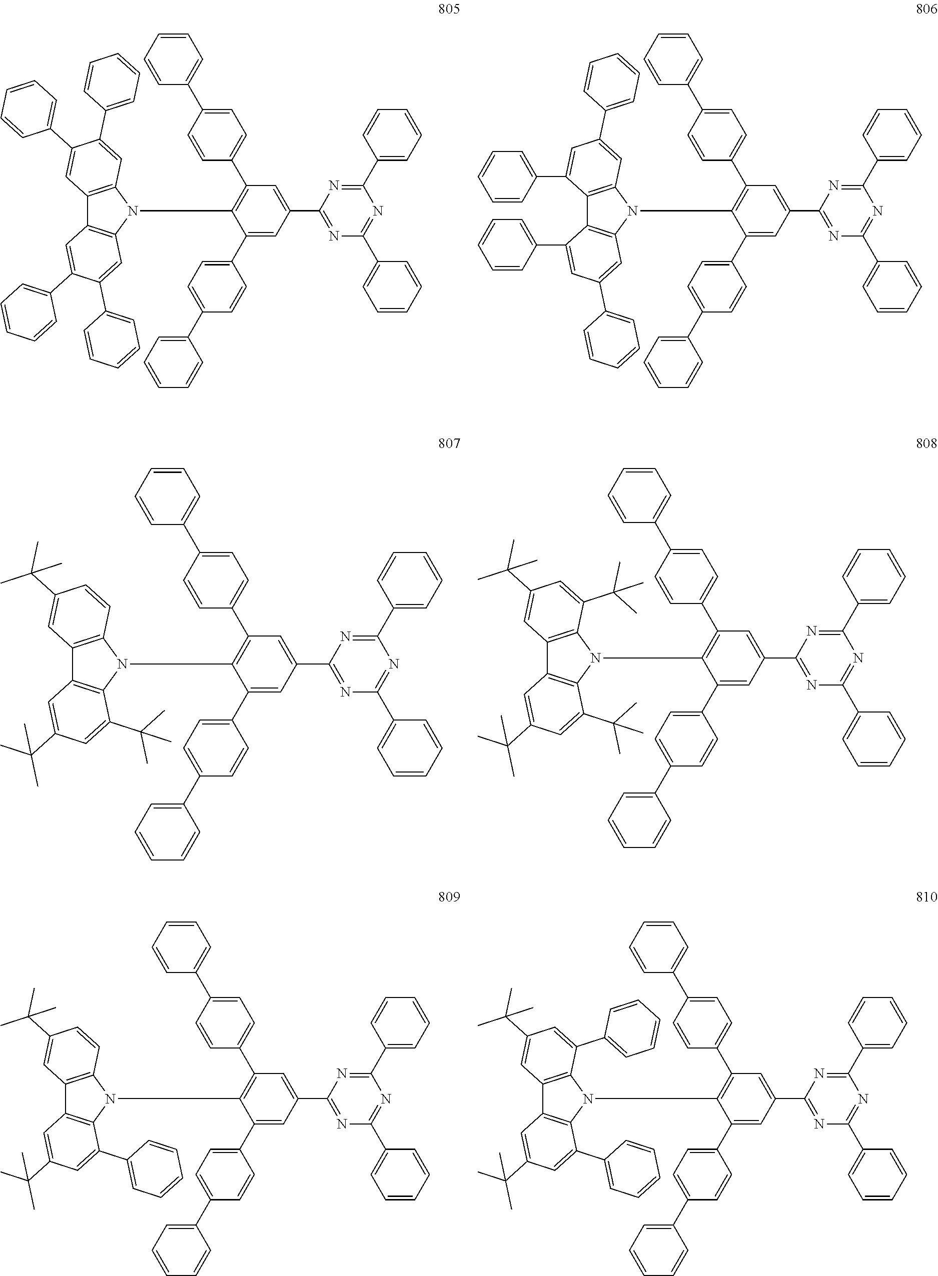

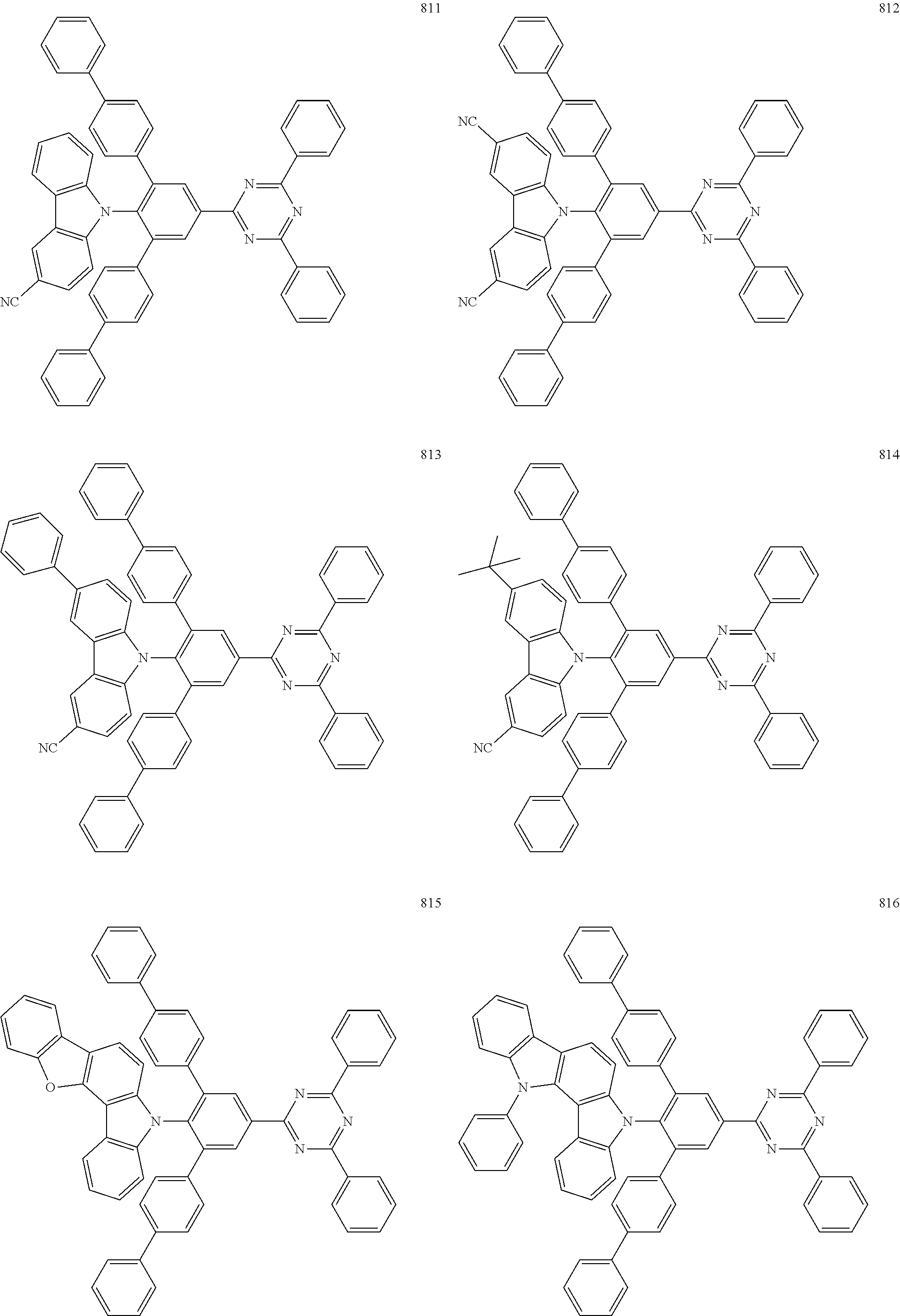

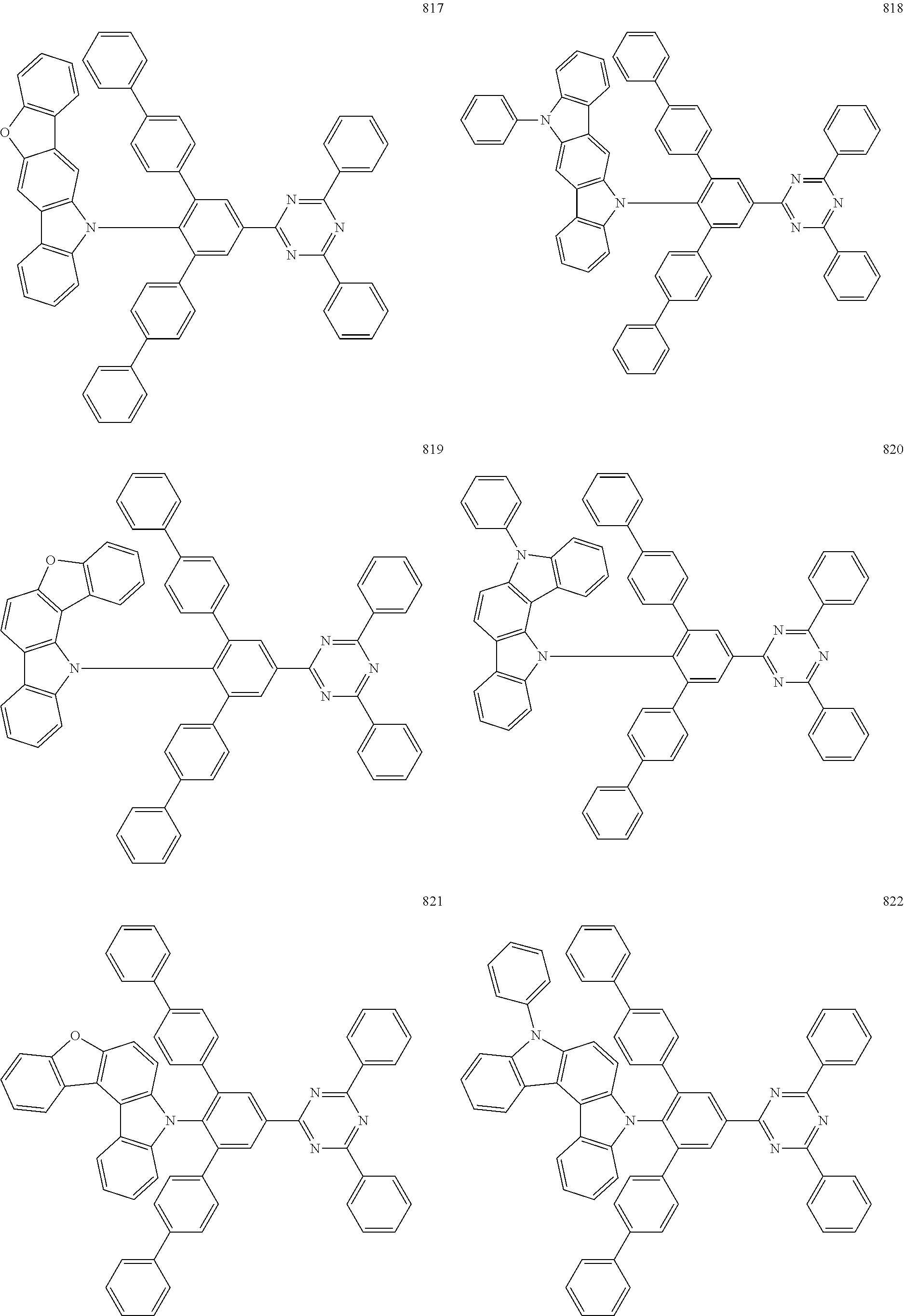

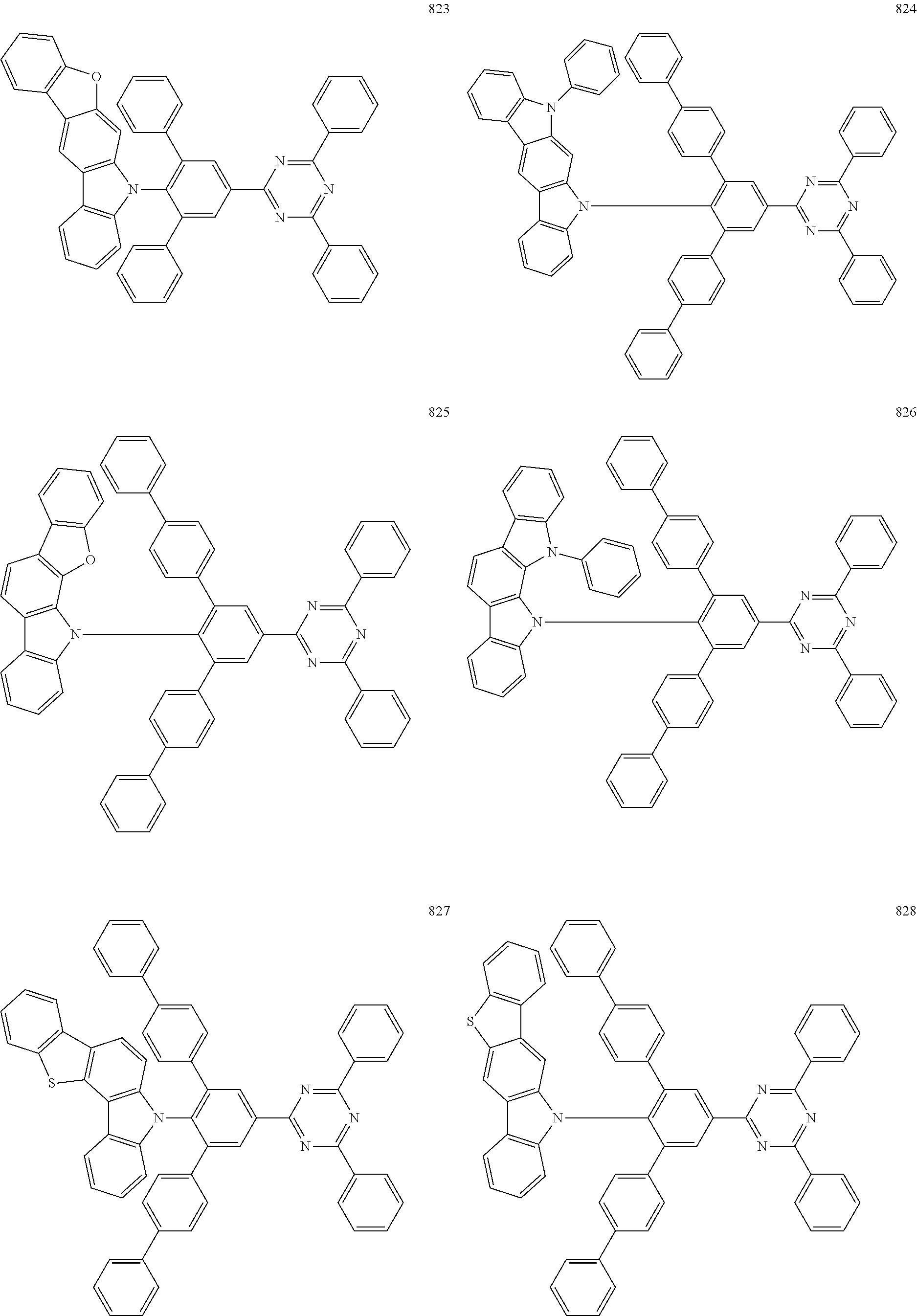

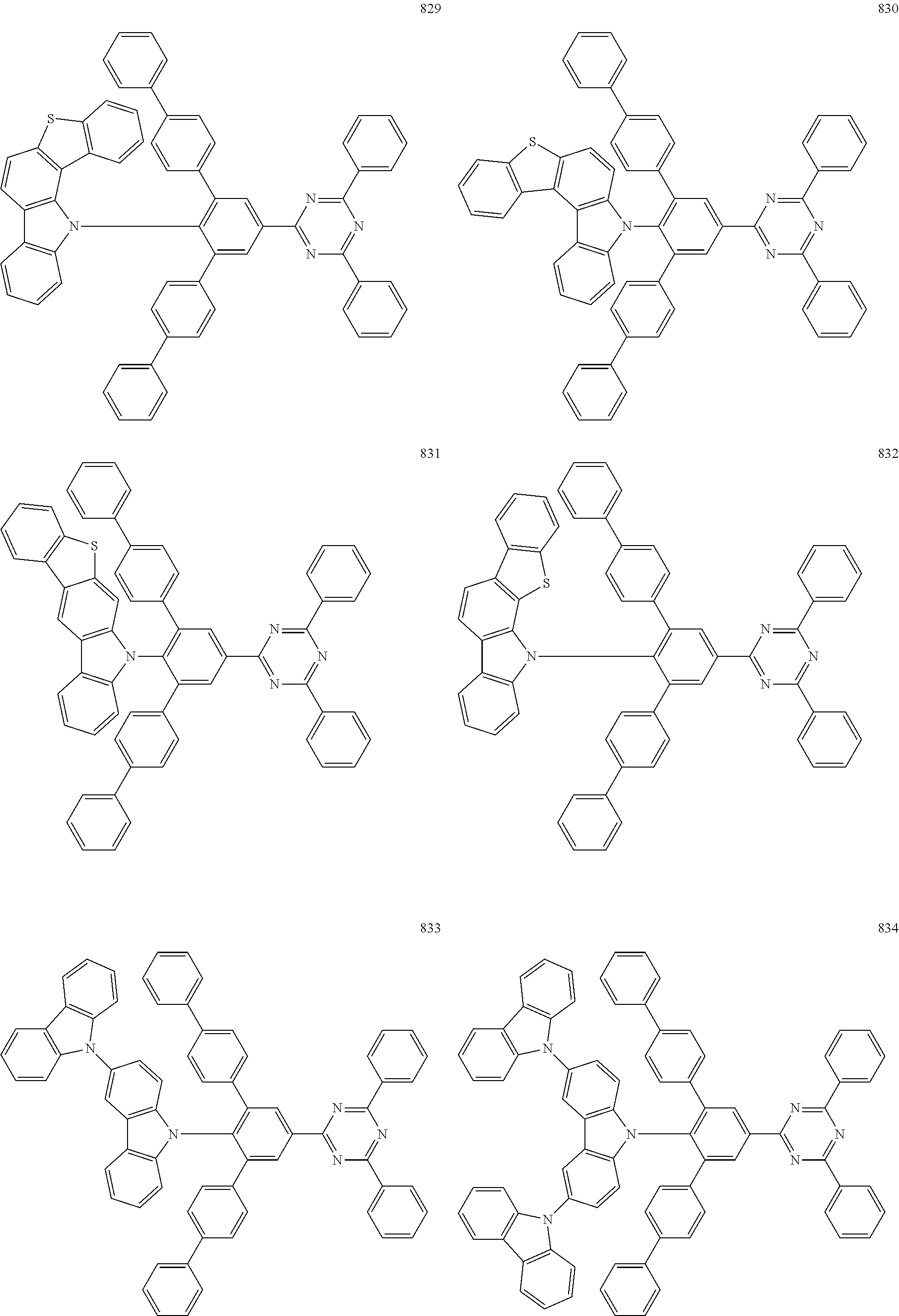

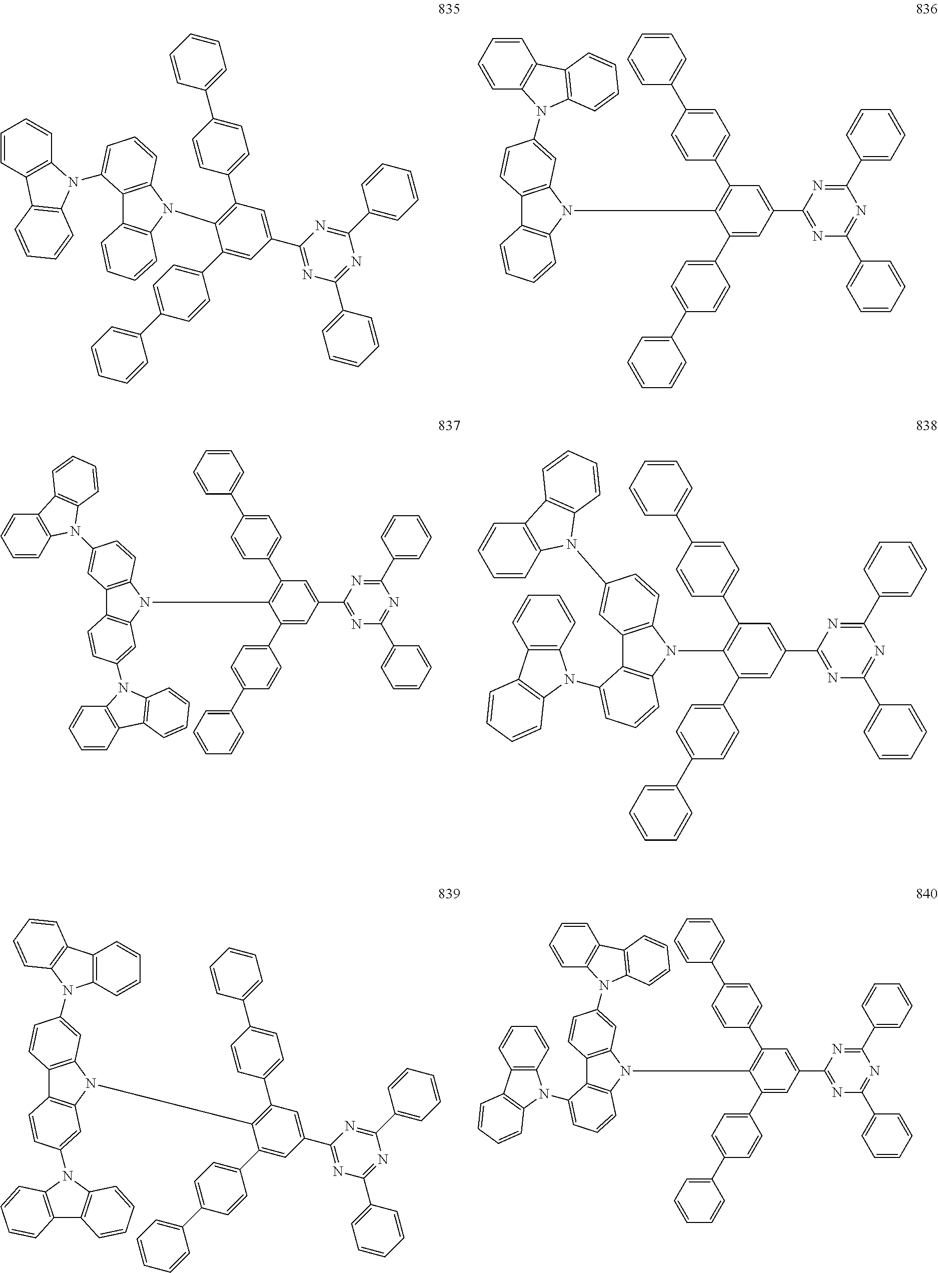

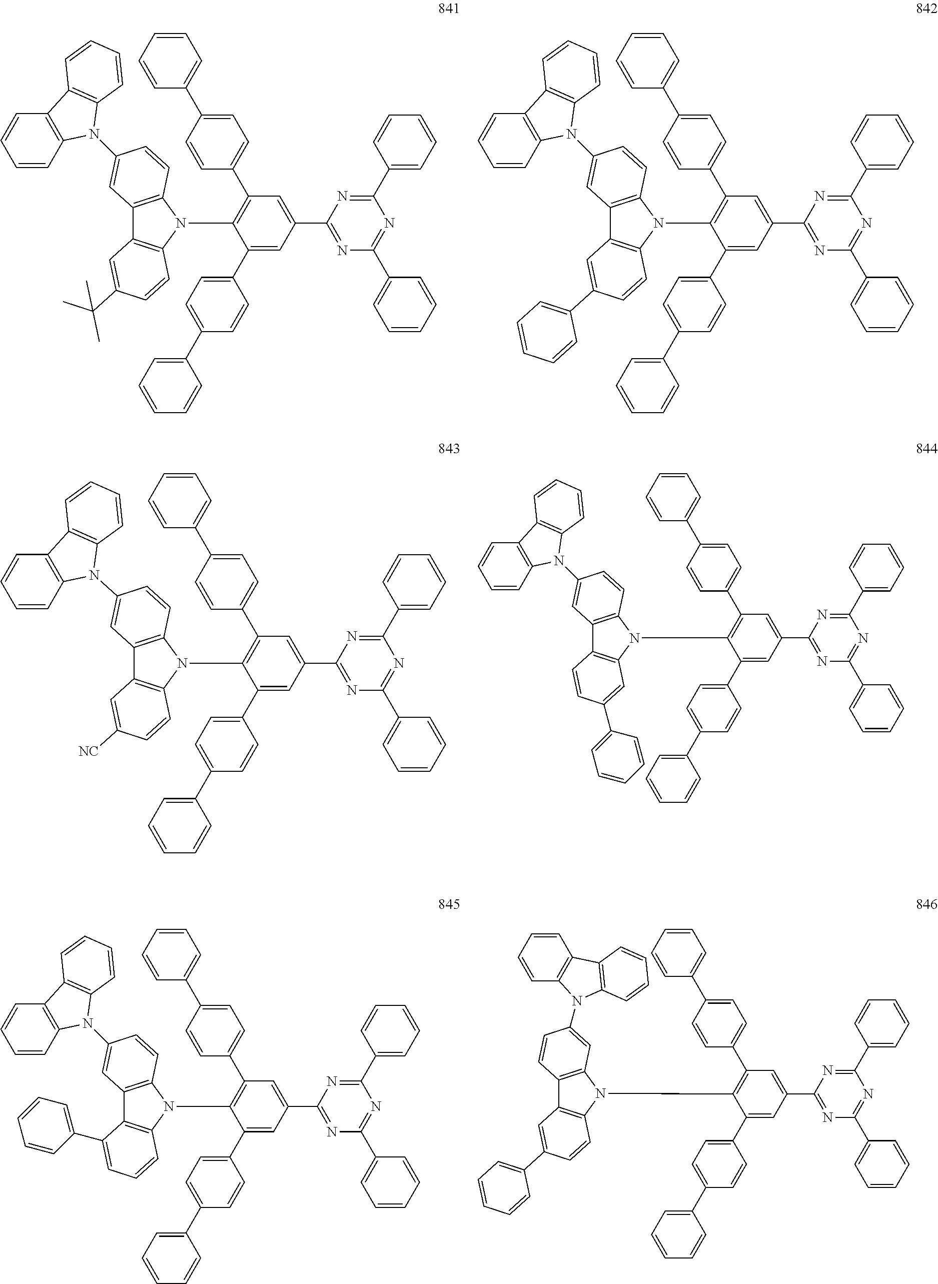

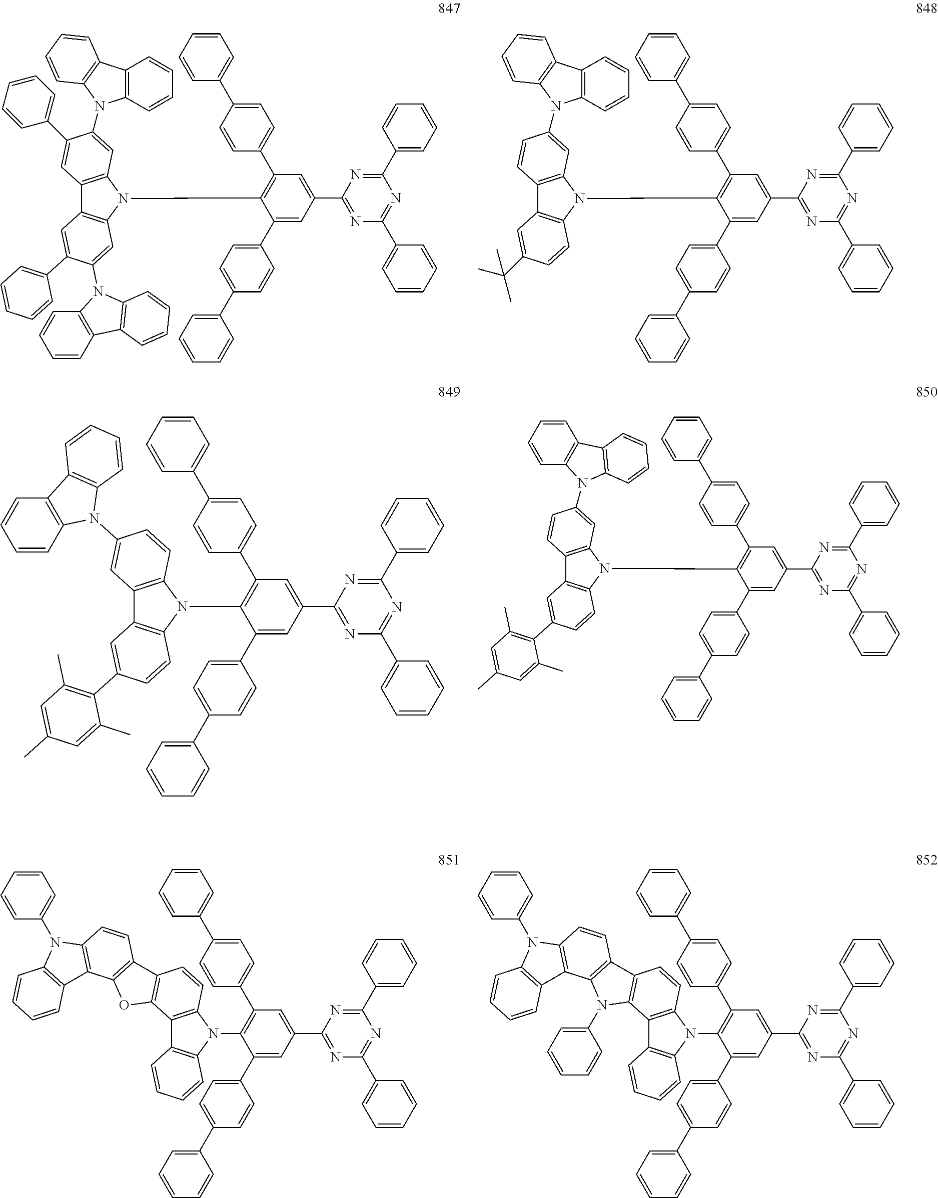

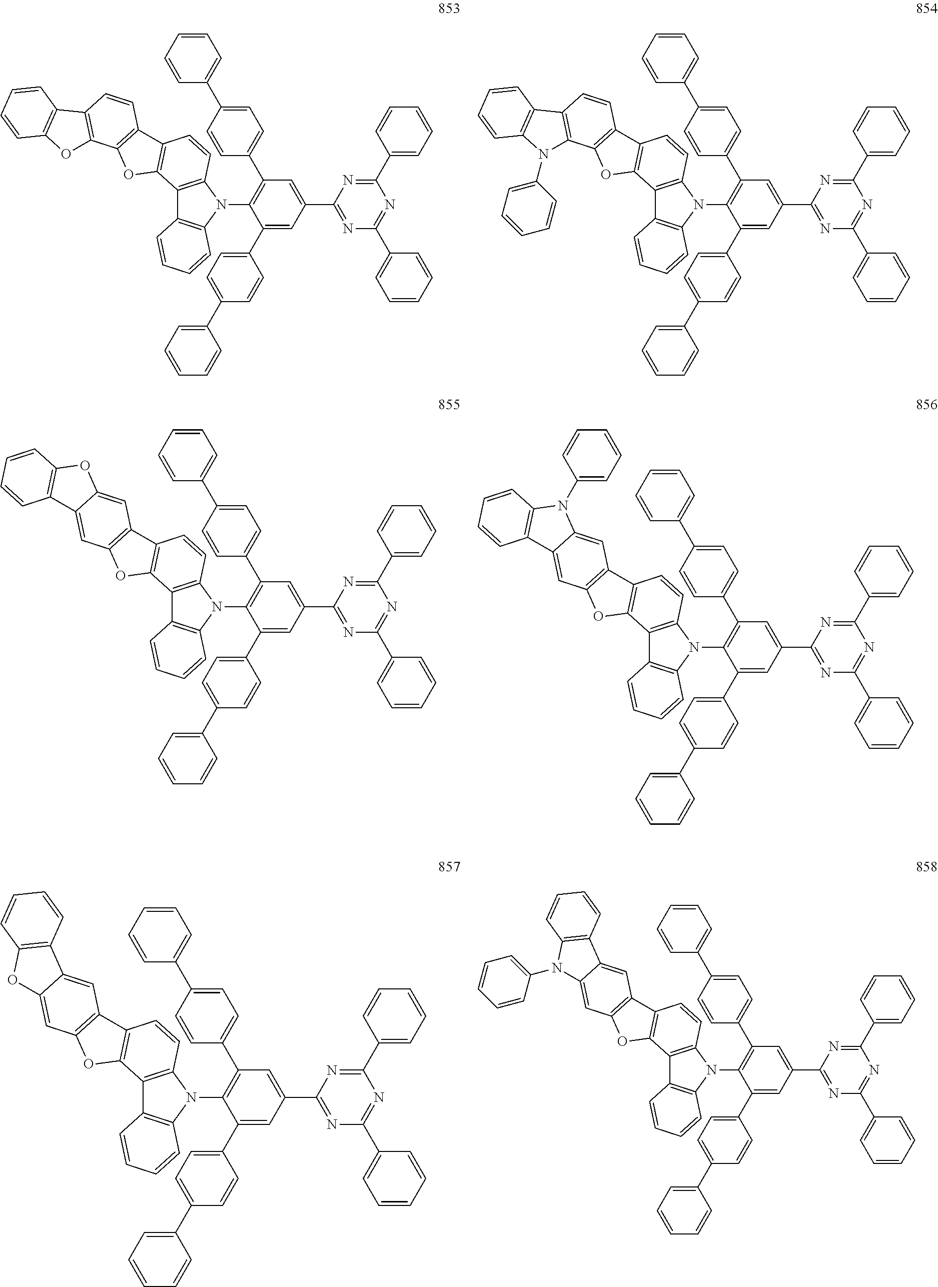

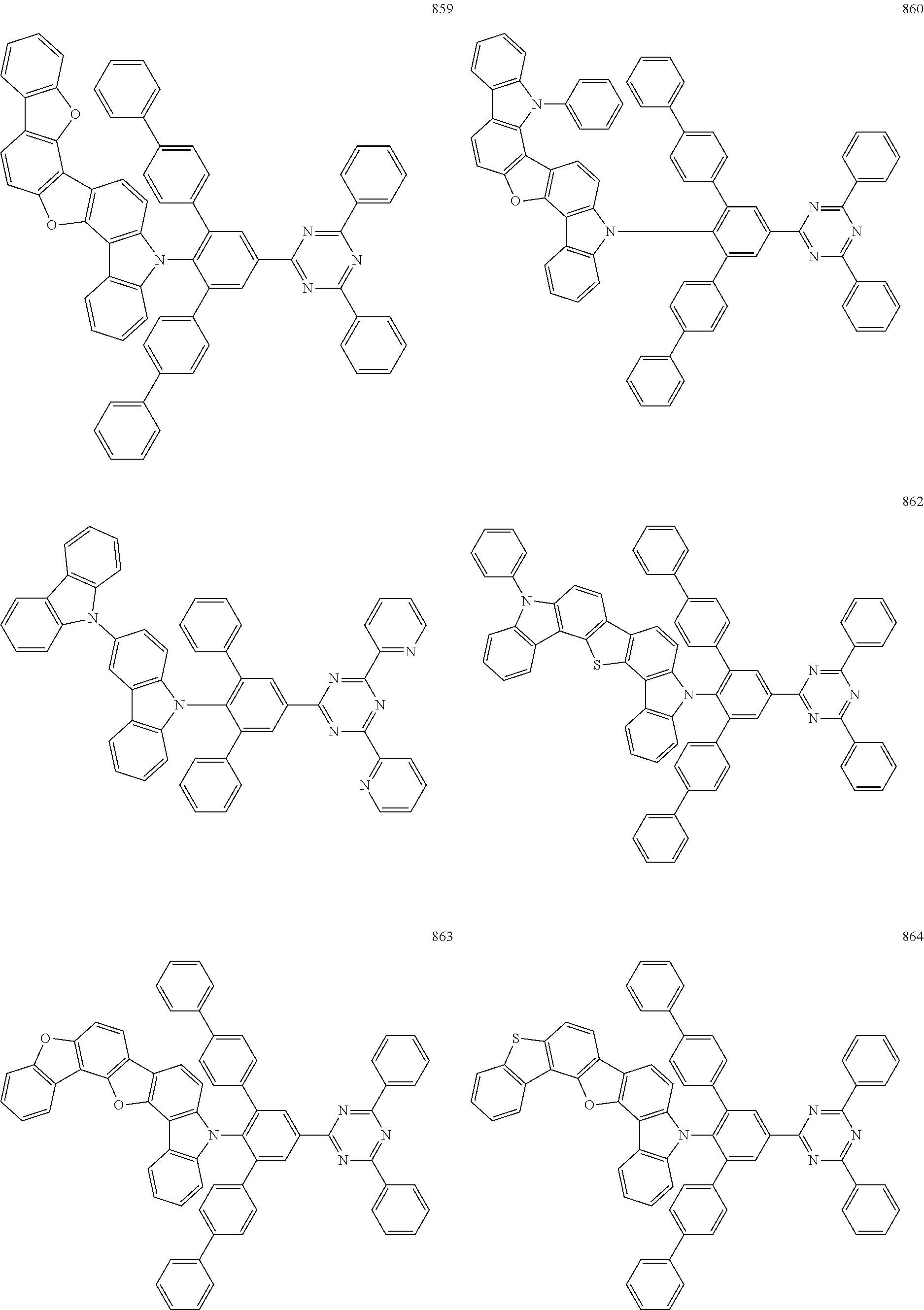

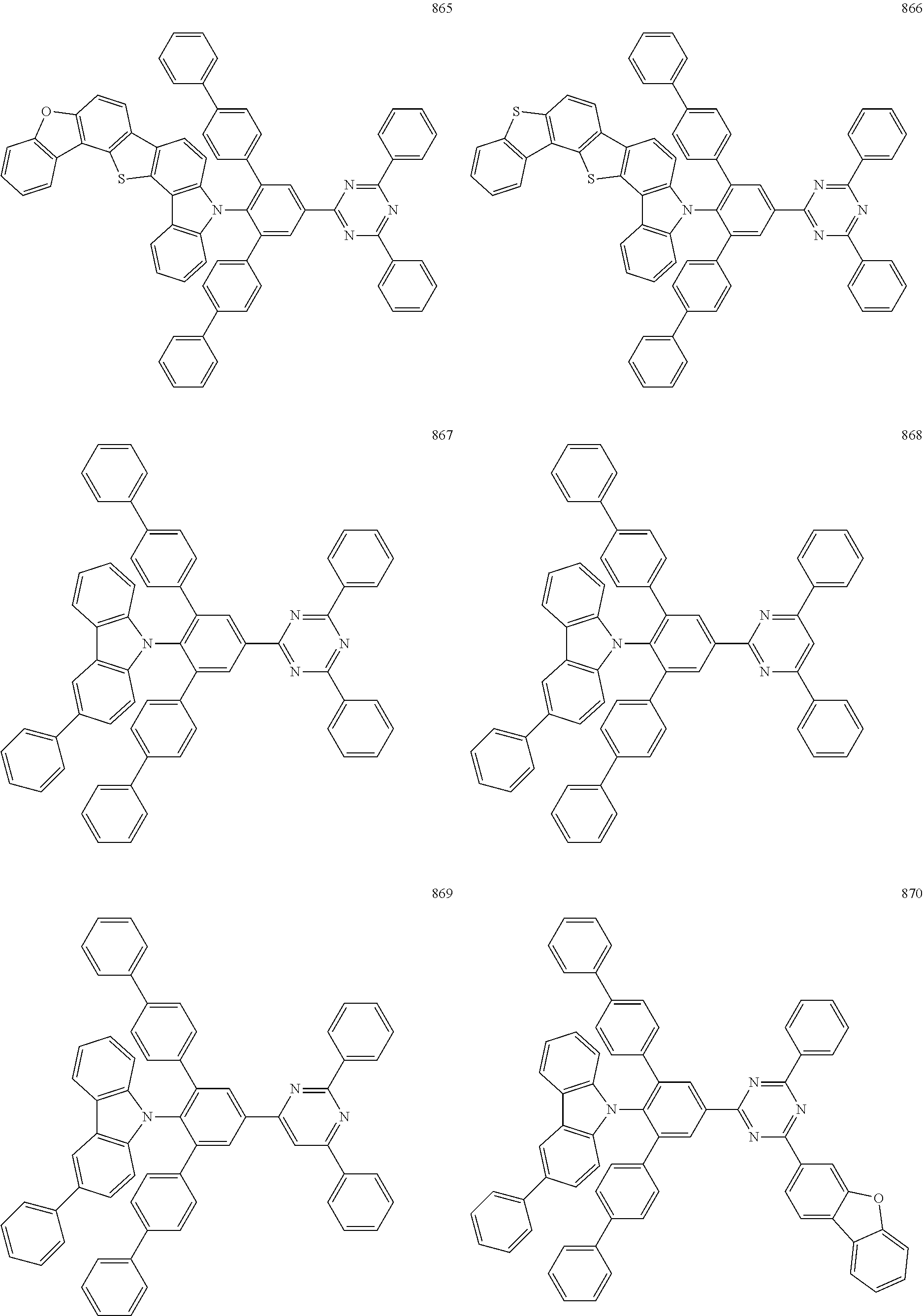

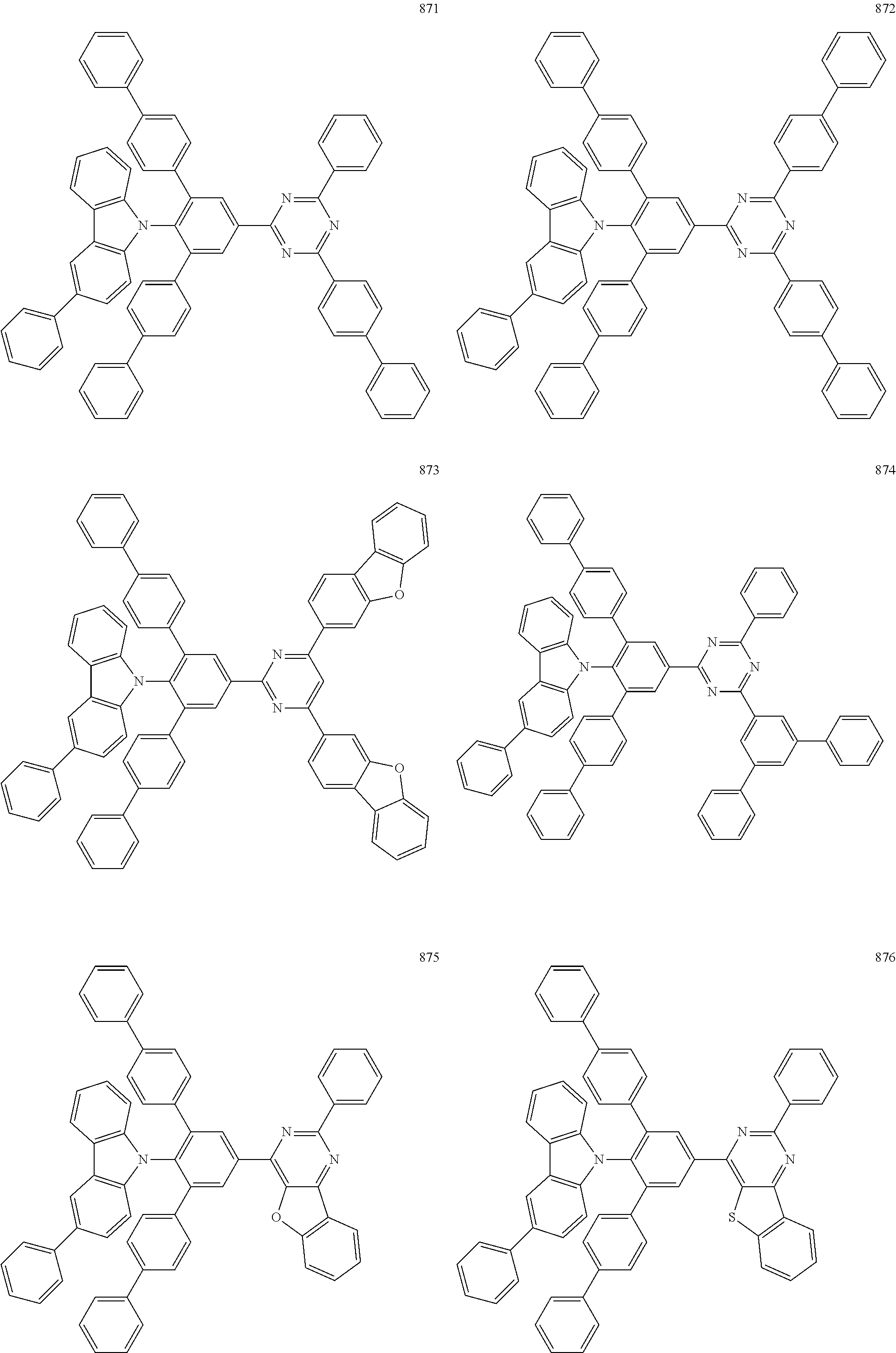

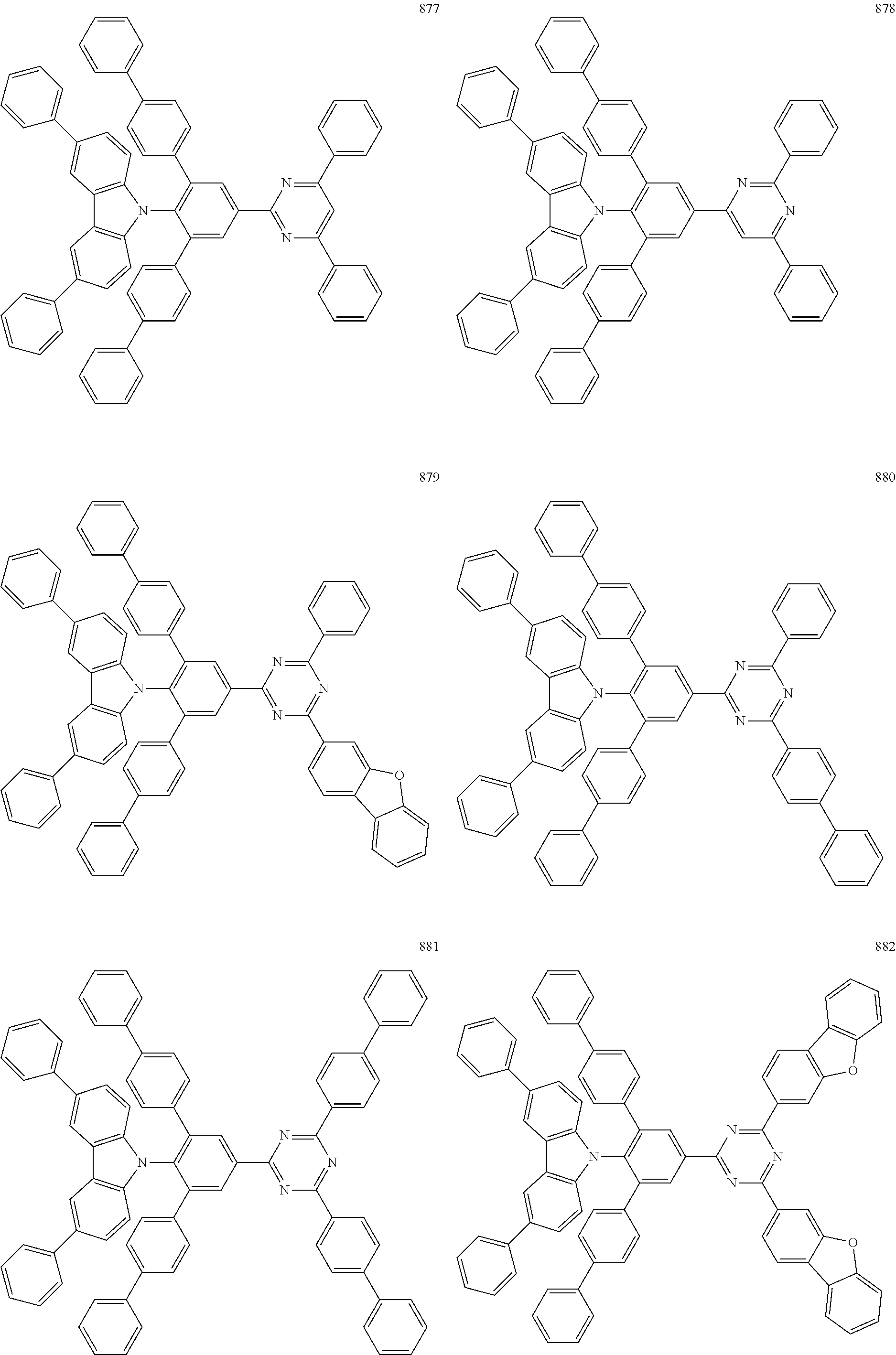

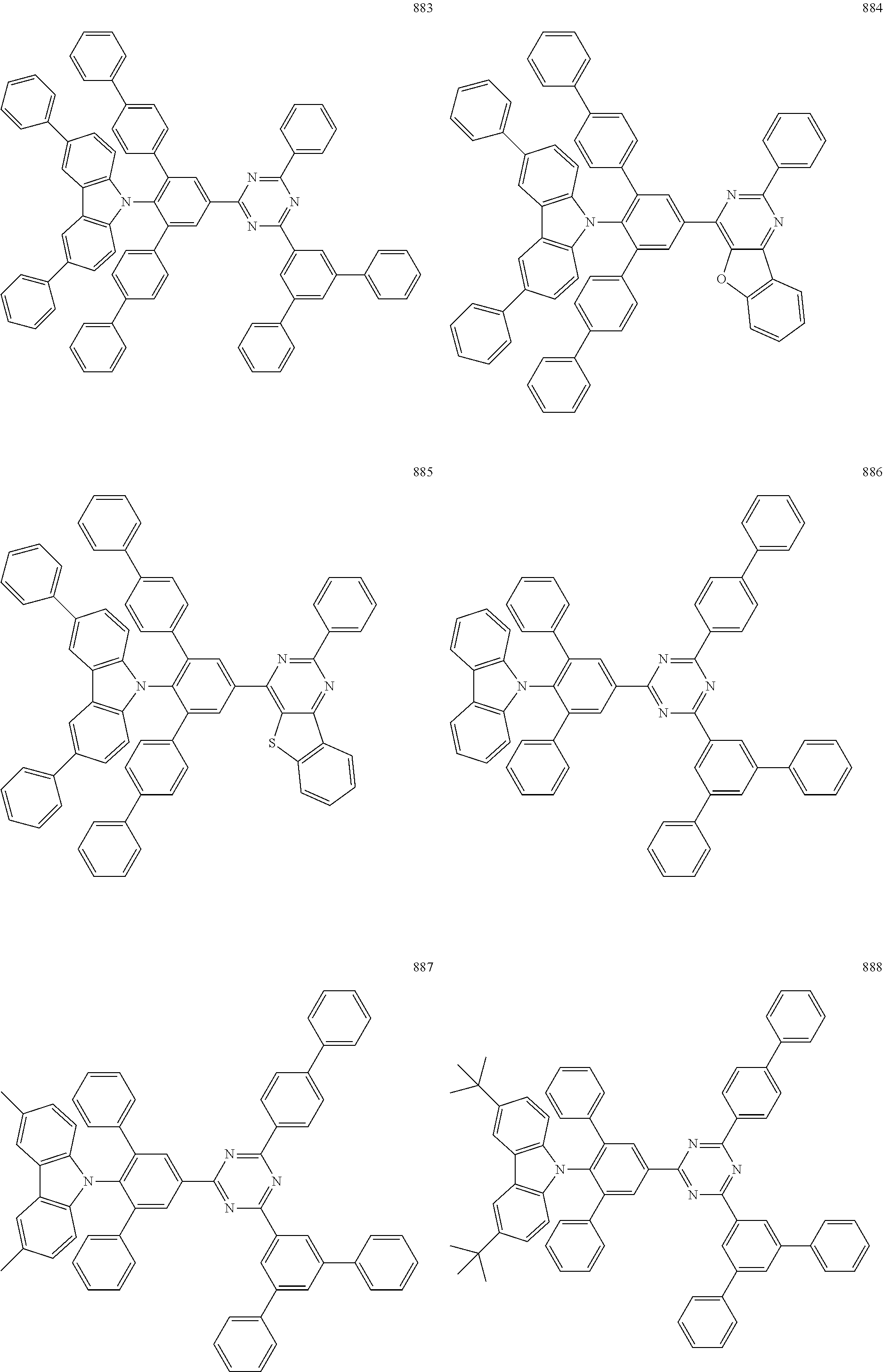

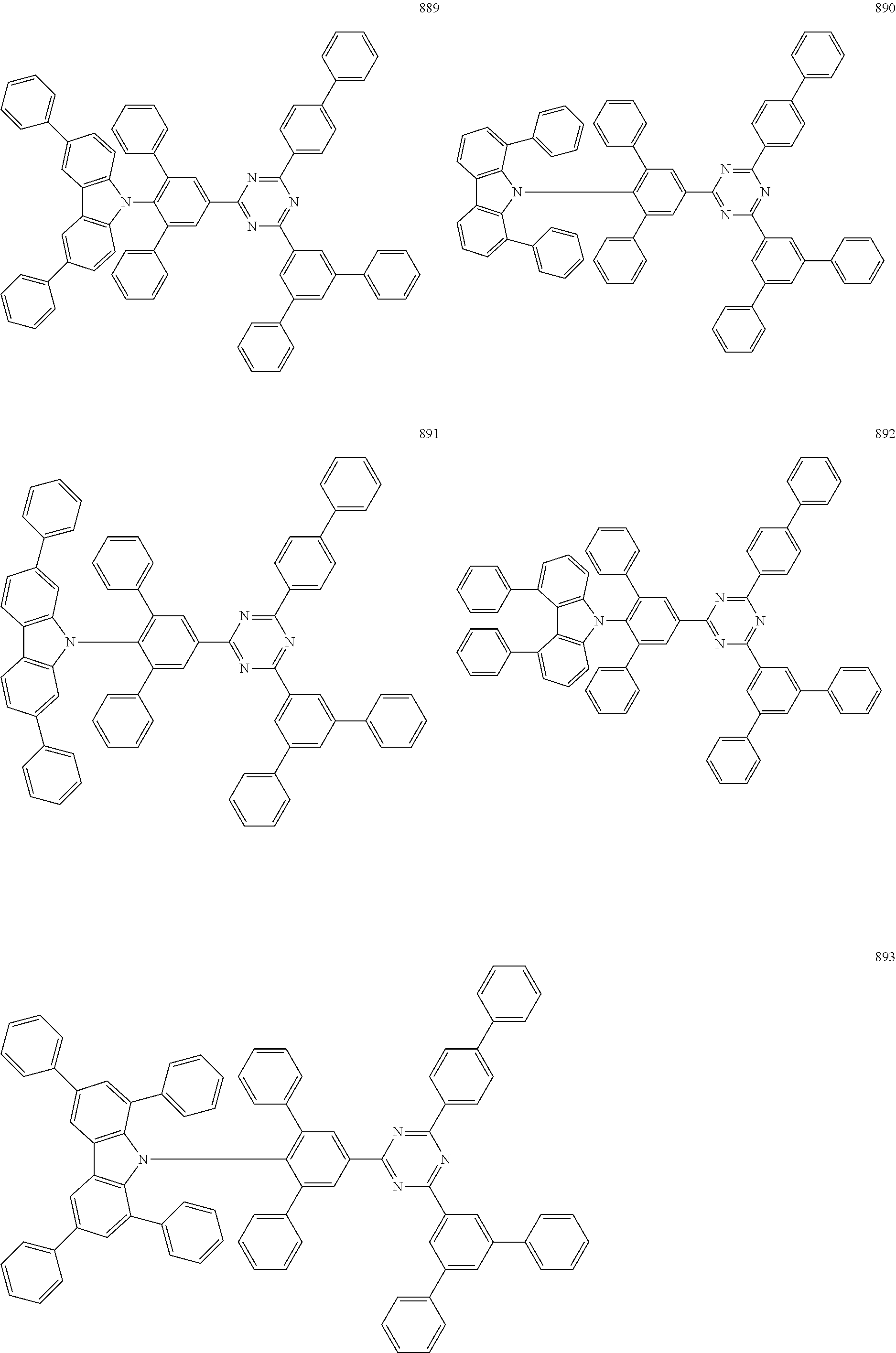

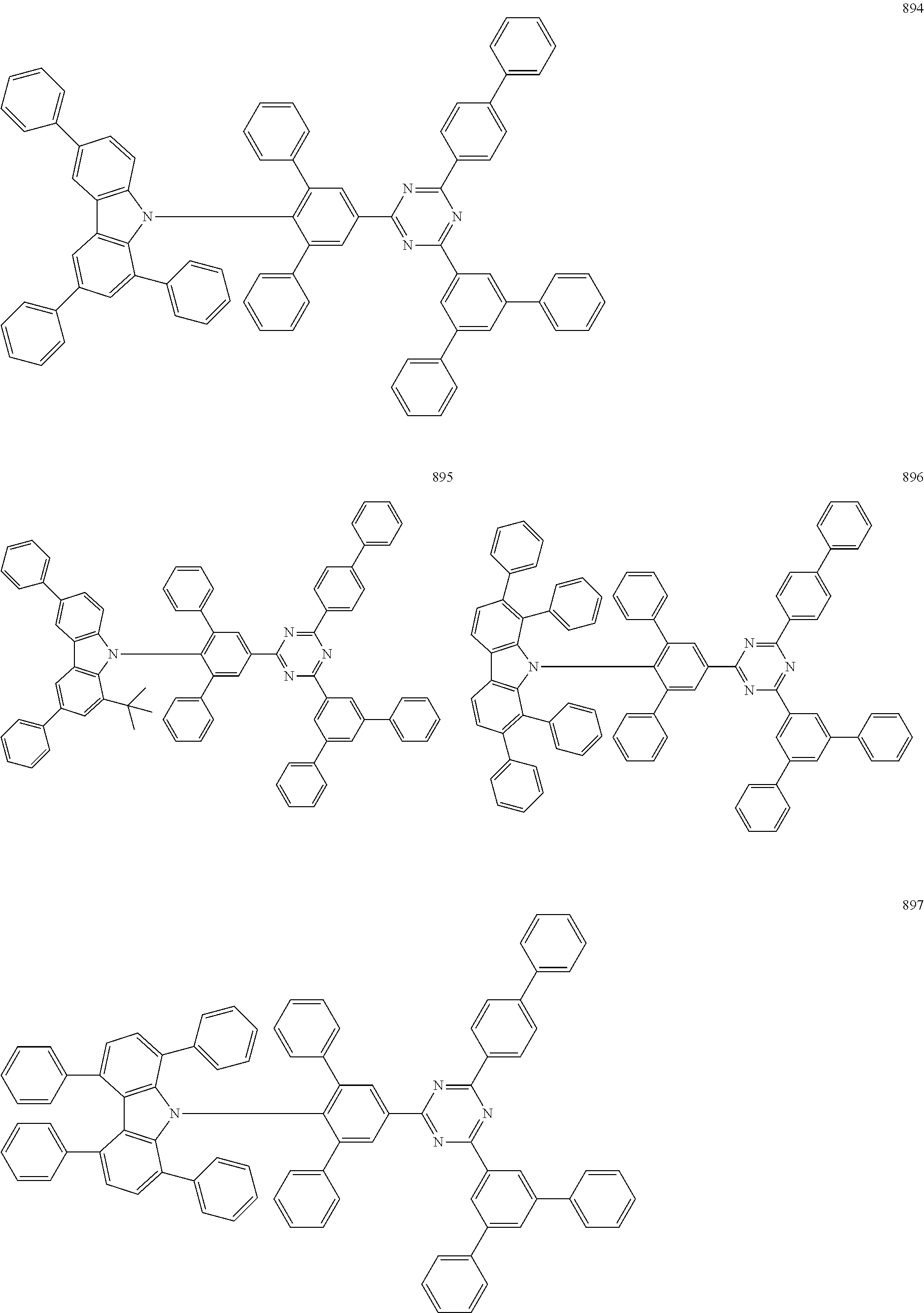

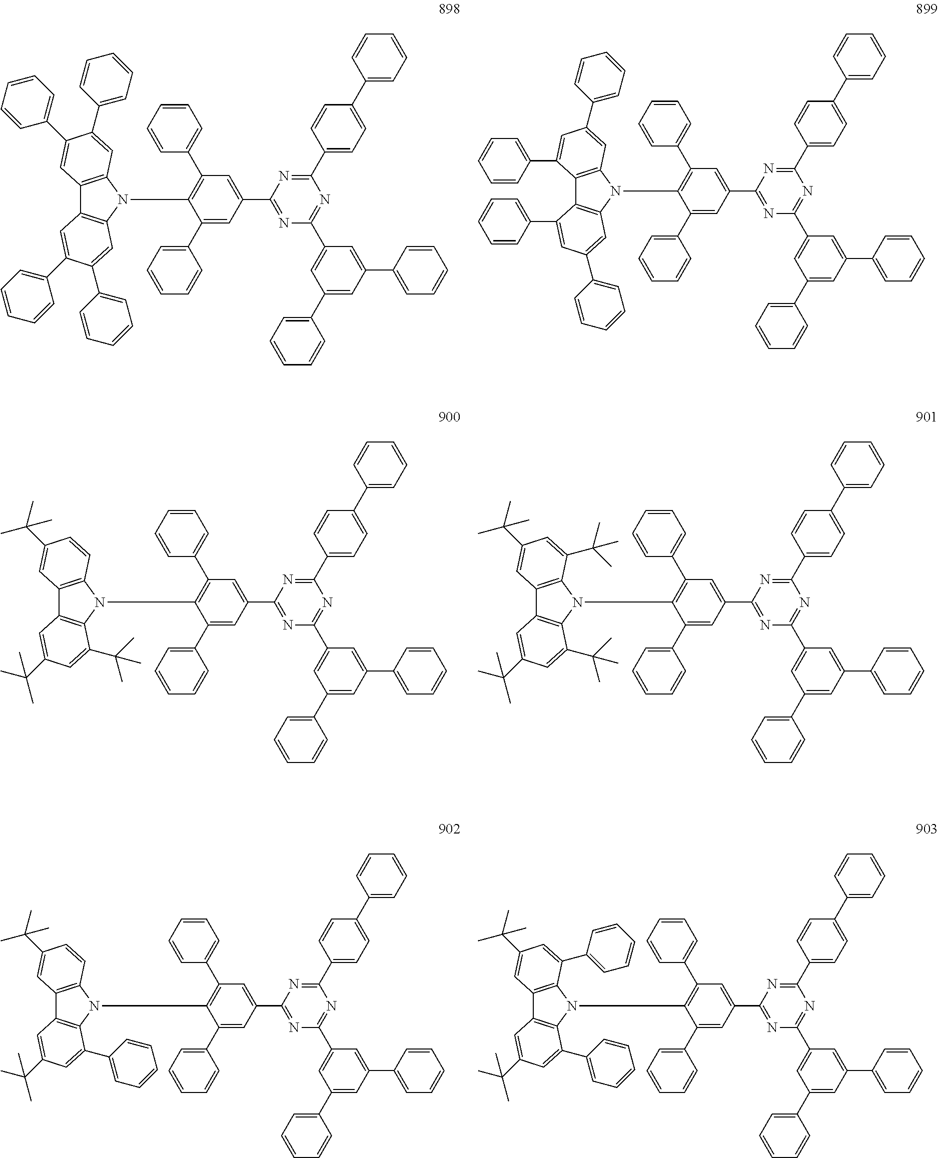

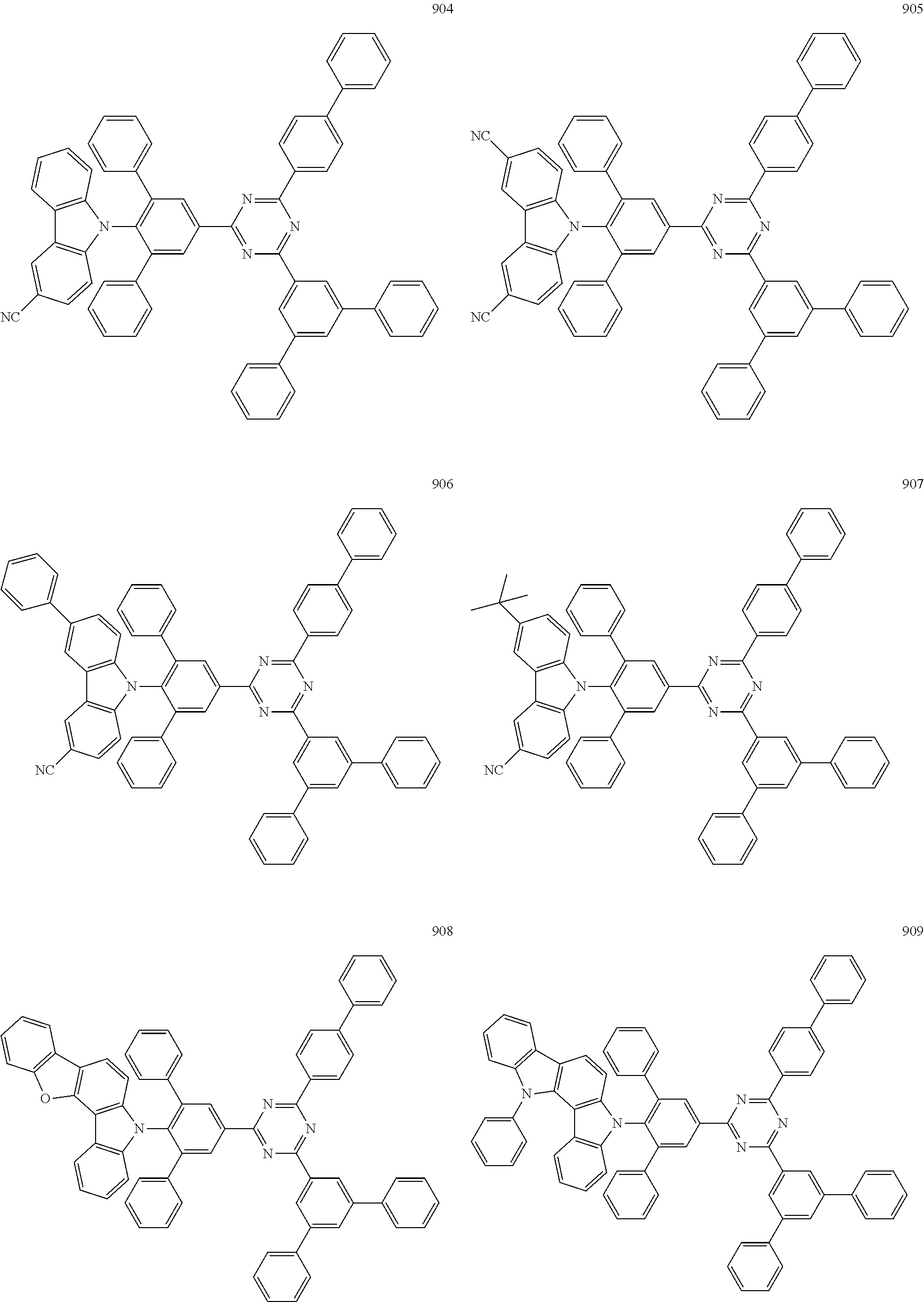

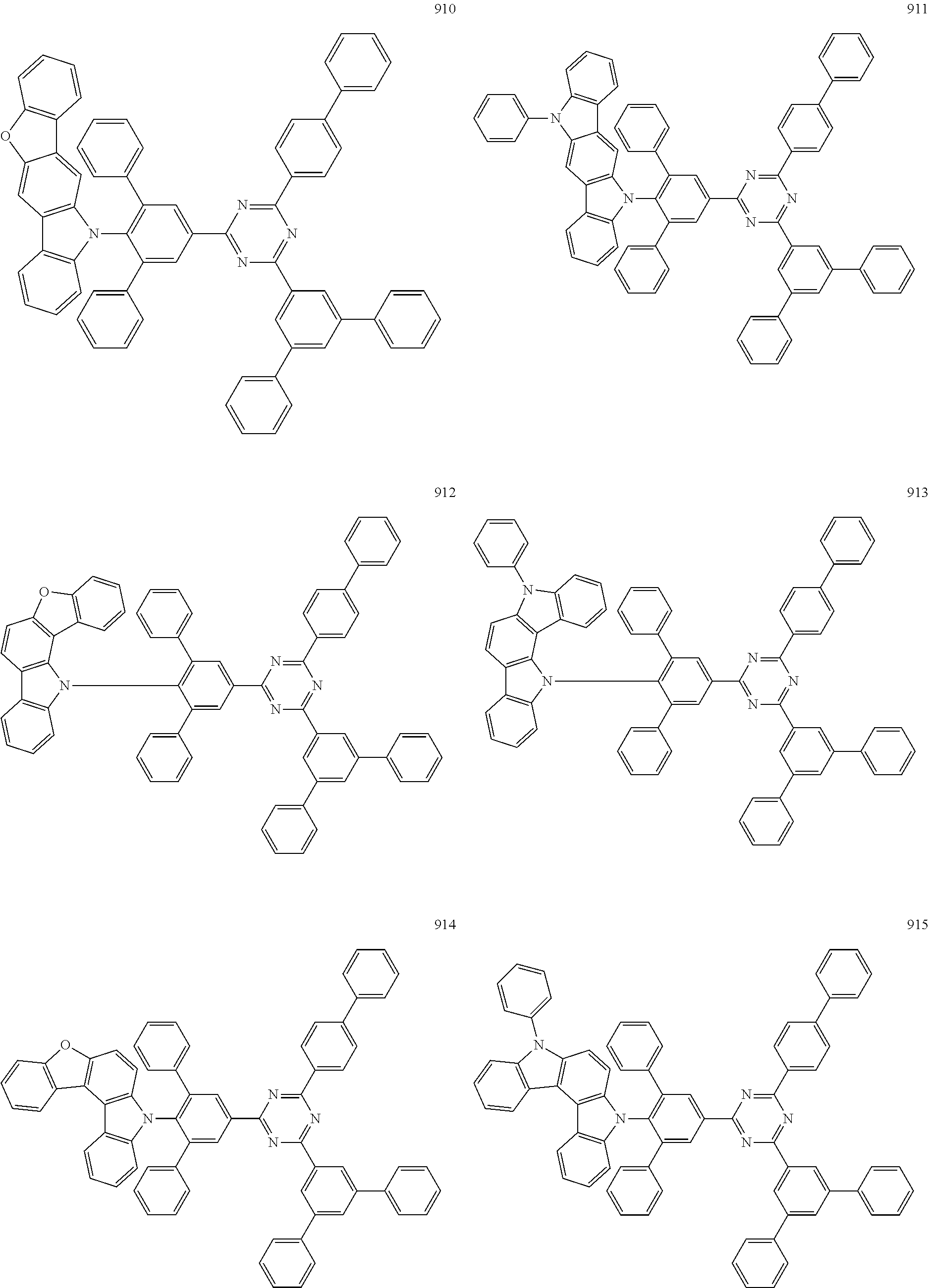

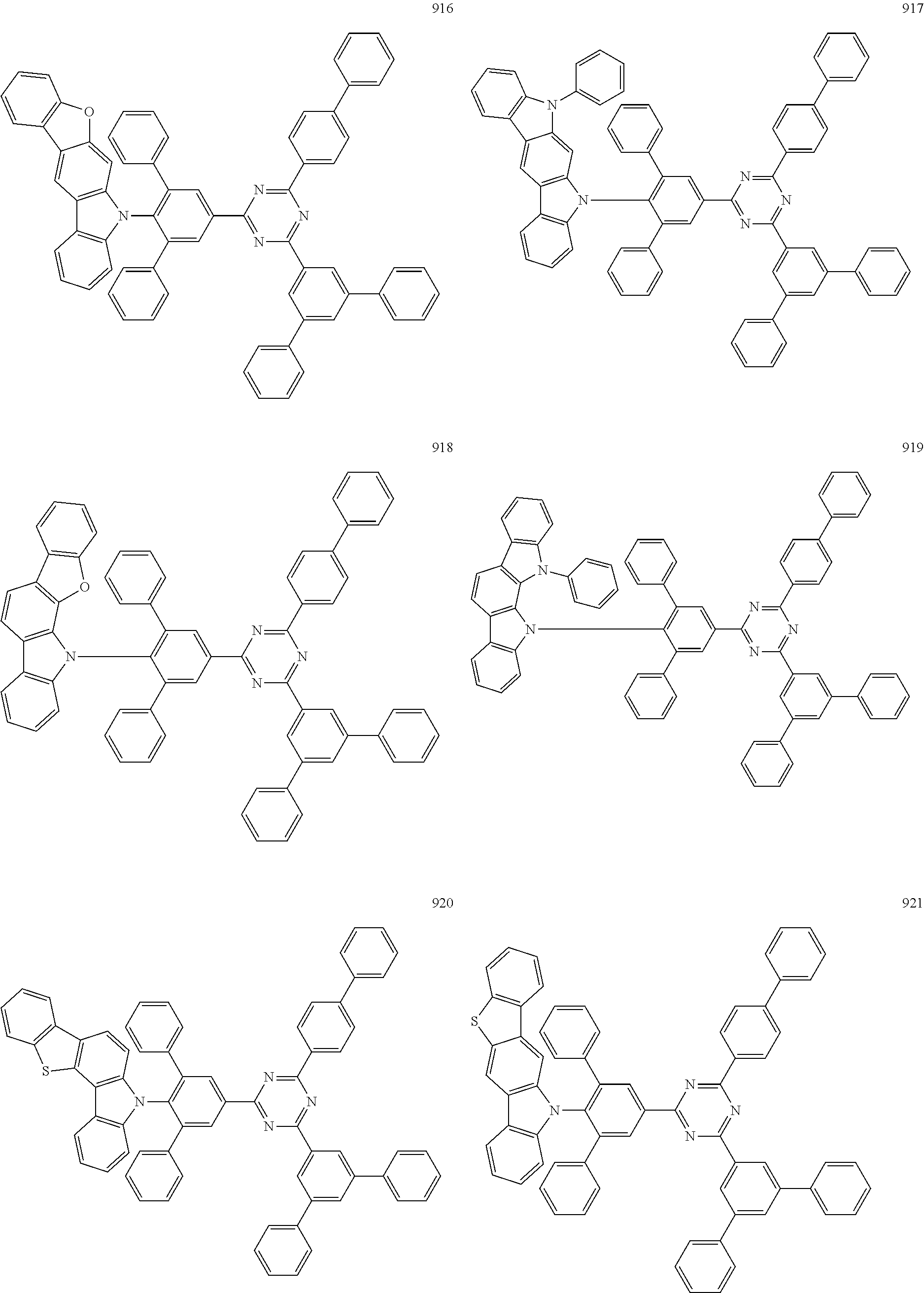

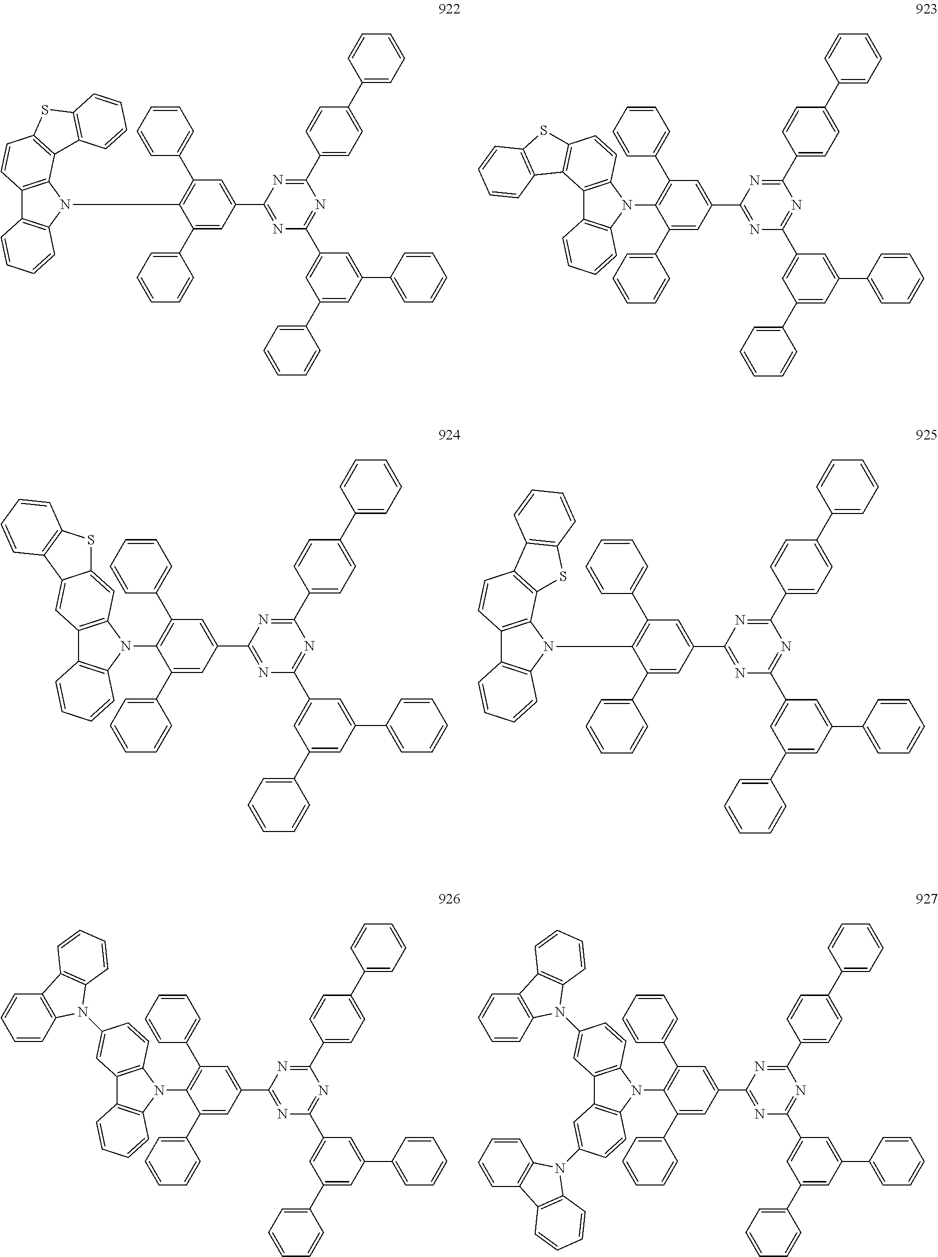

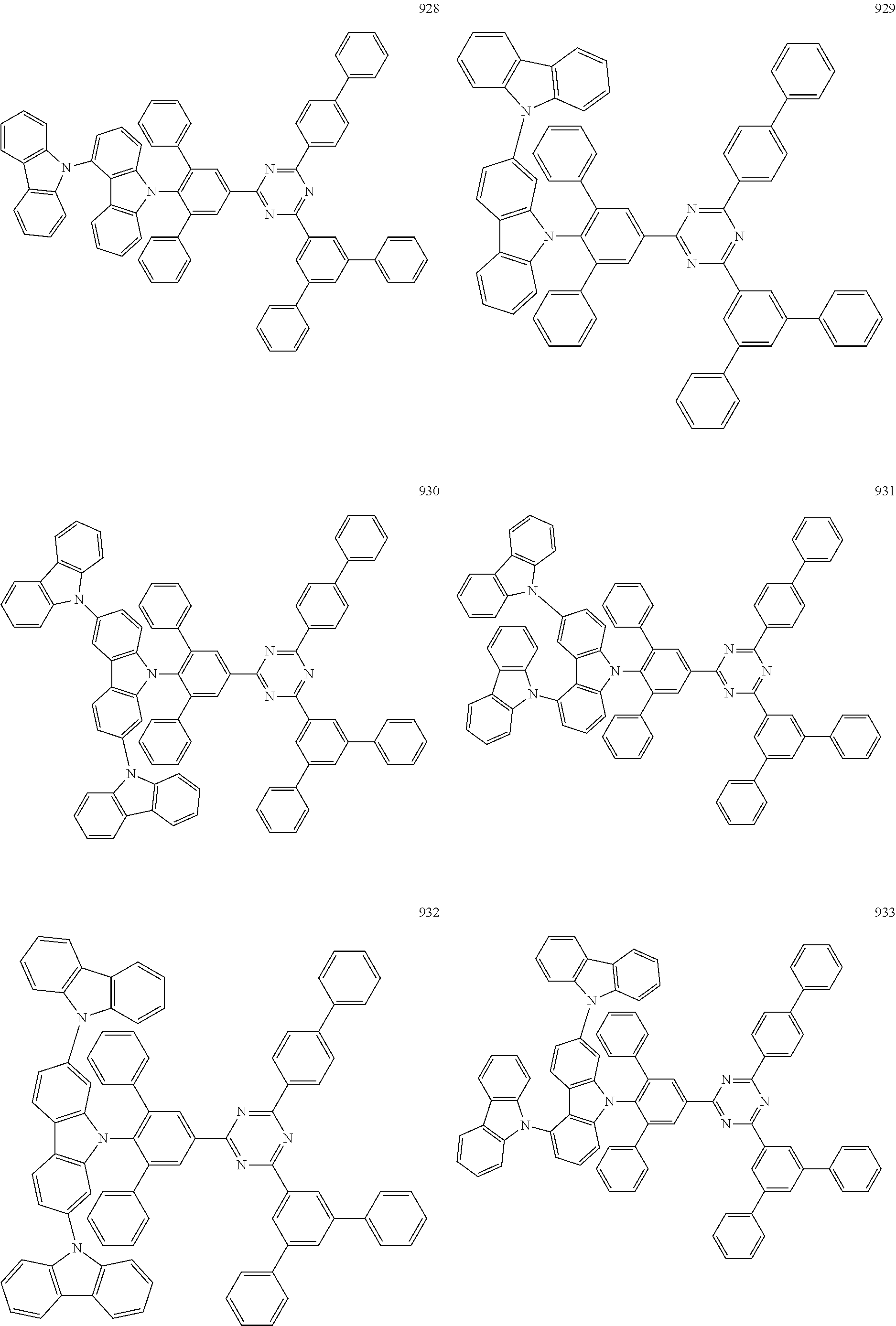

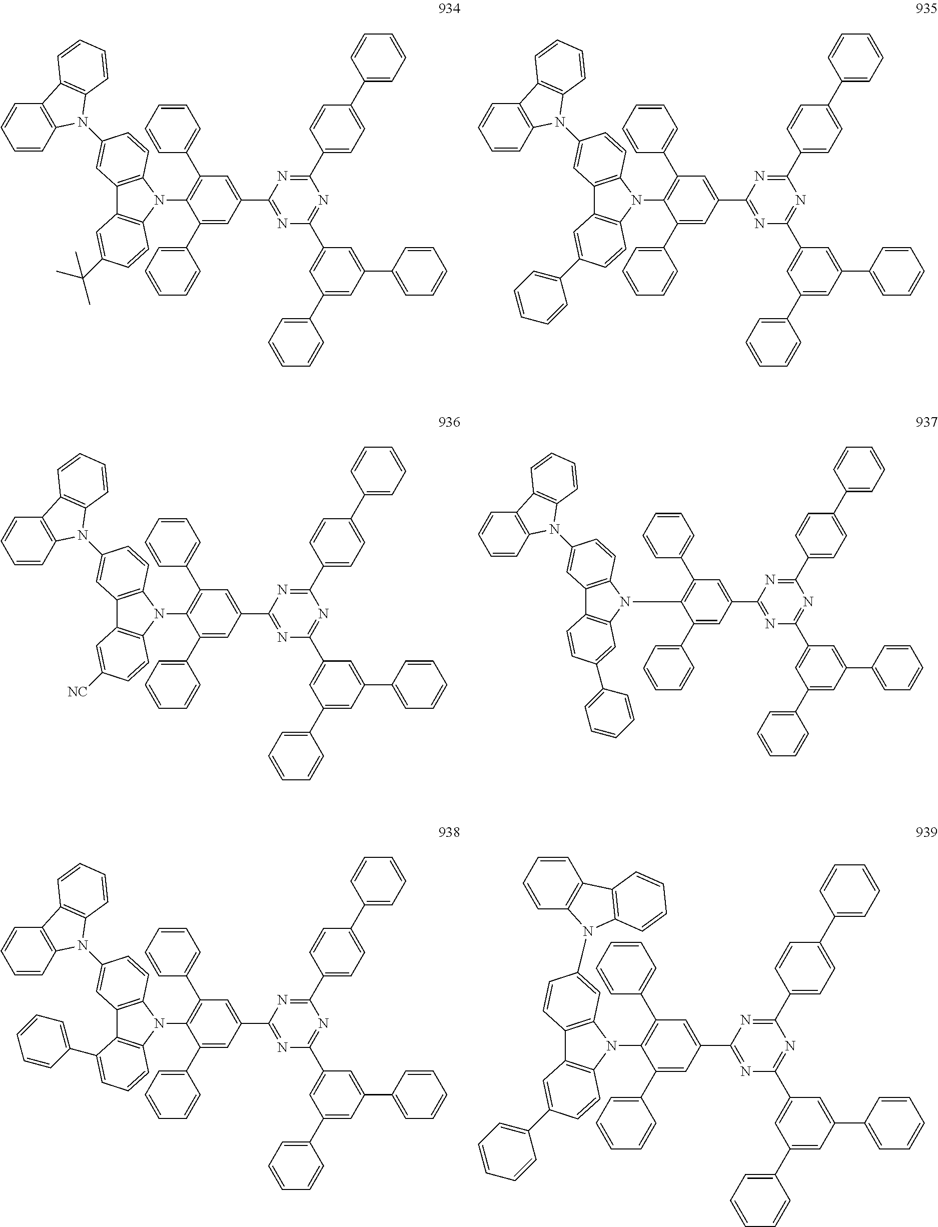

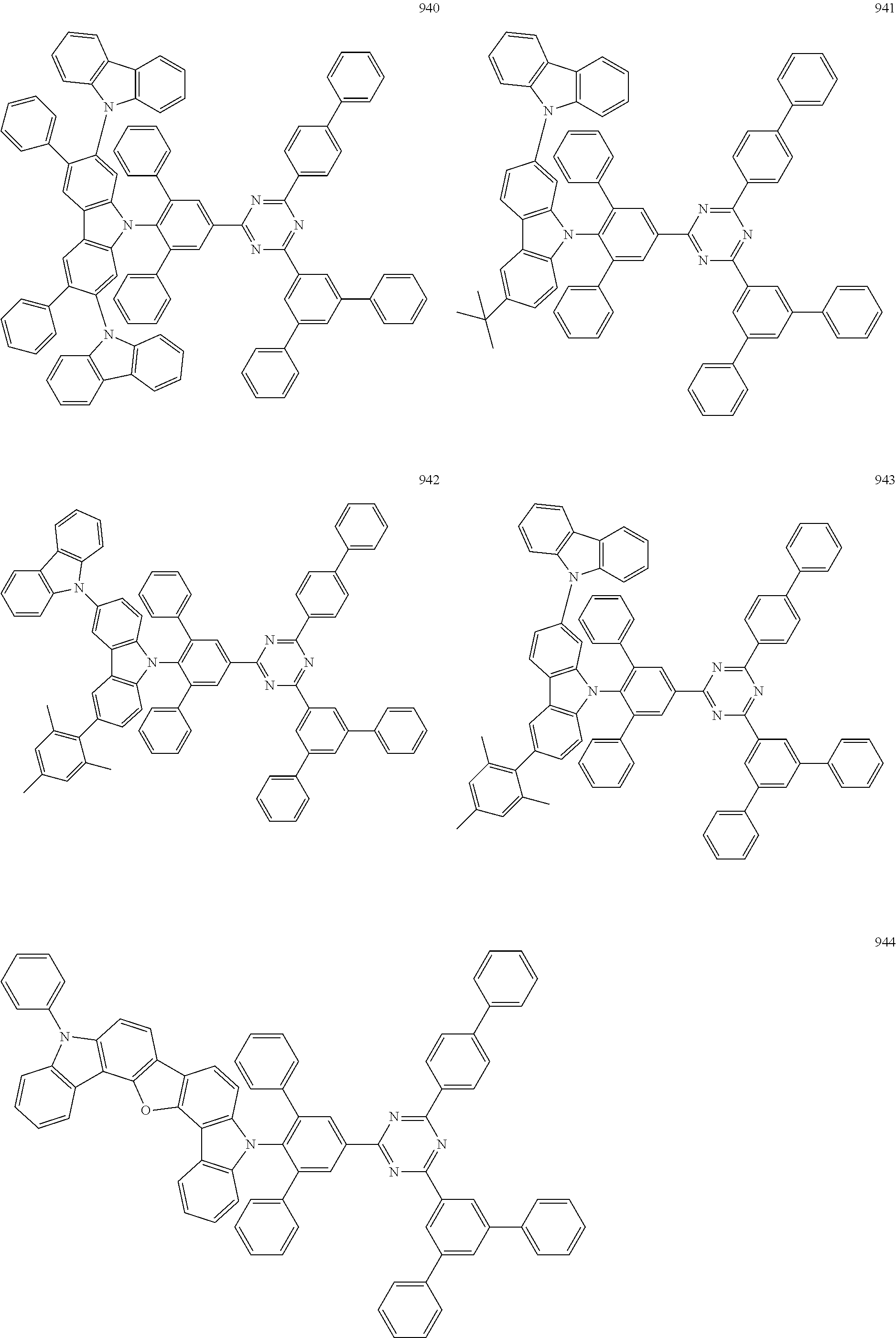

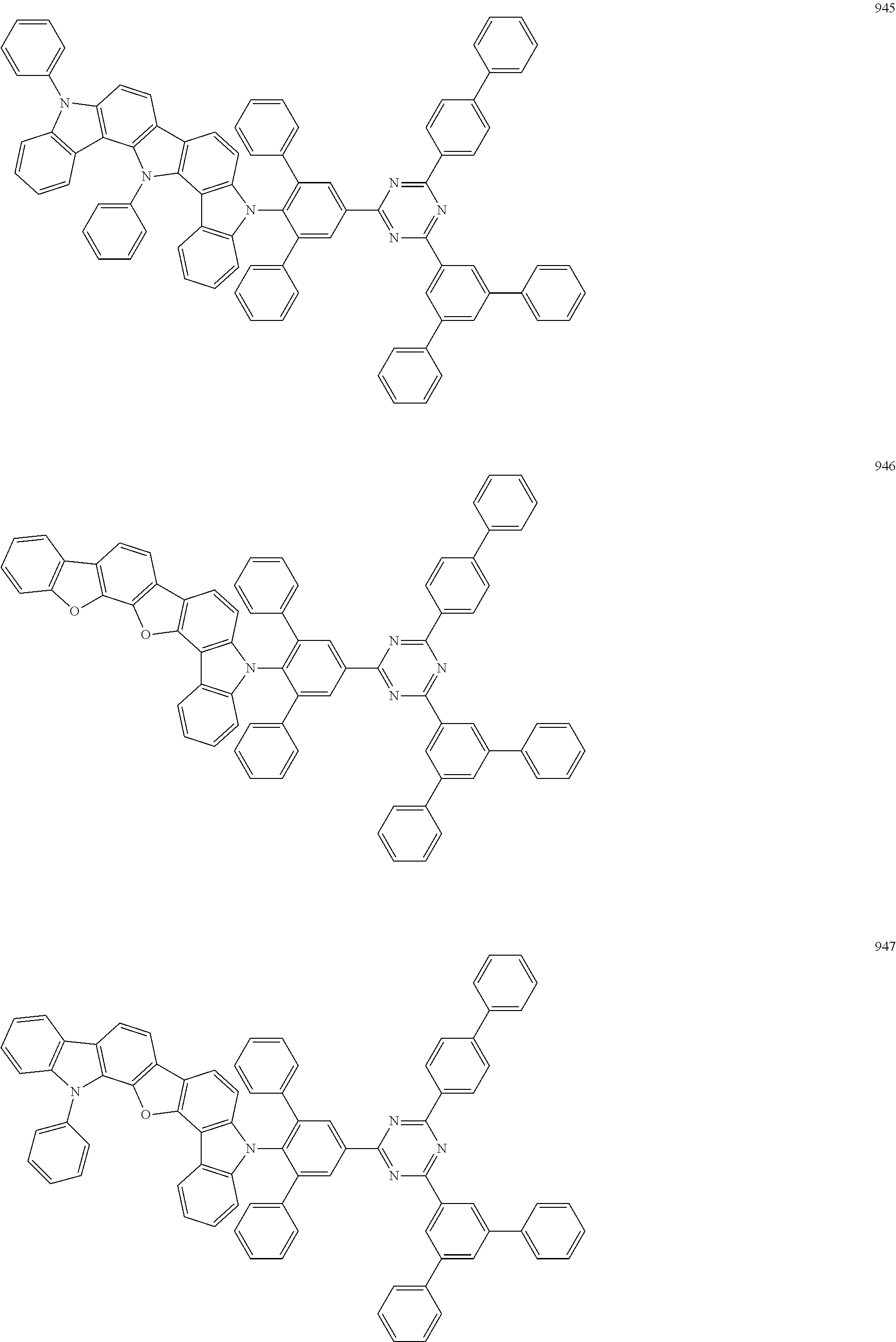 [SMILES: c1ccc(-c2ccc(-c3nc(-c4cc(-c5ccccc5)cc(-c5ccccc5)c4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10oc98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10n(-c%10ccccc%10)c98)c7ccc65)c(-c5ccccc5)c4)n3)cc2)cc1]

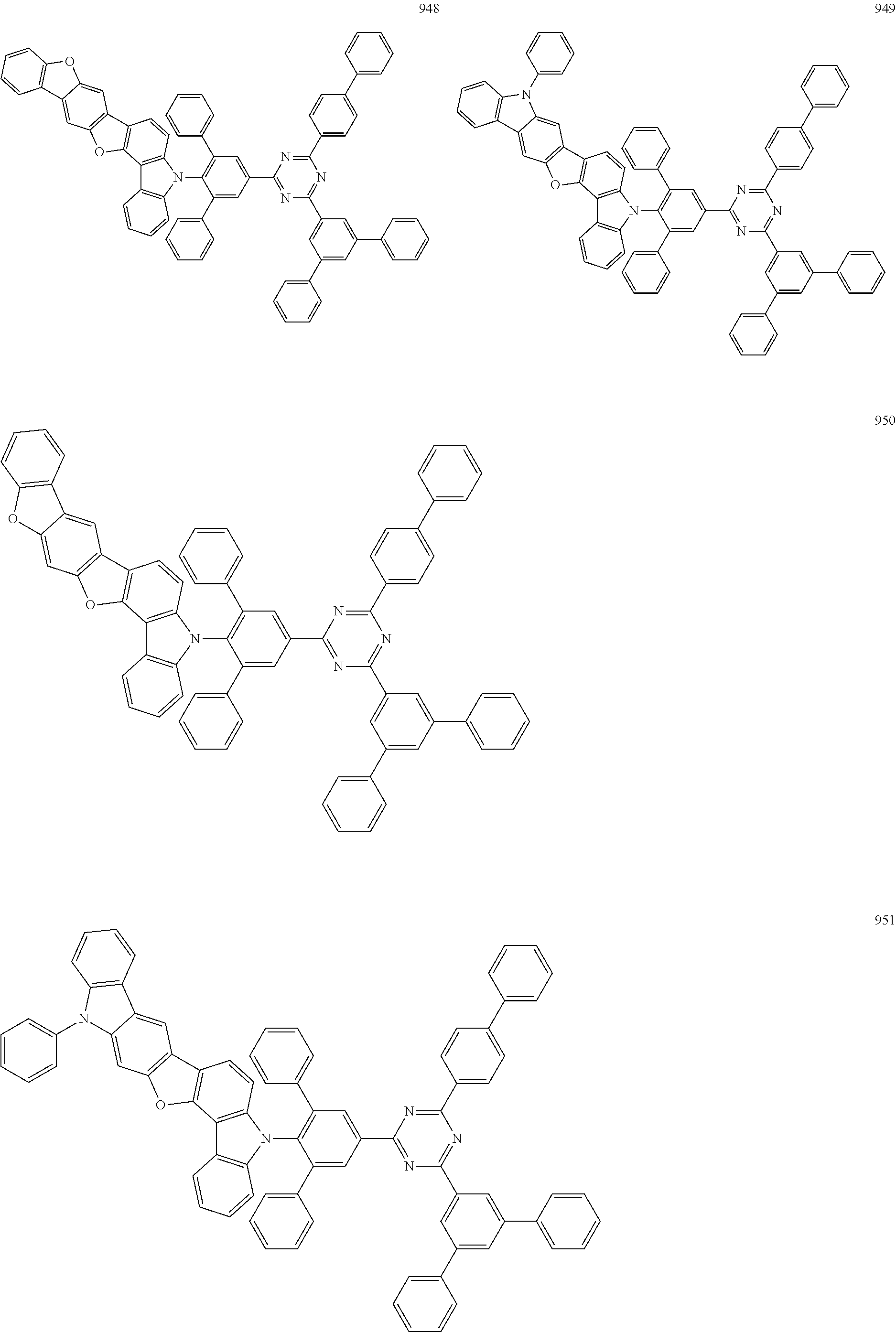

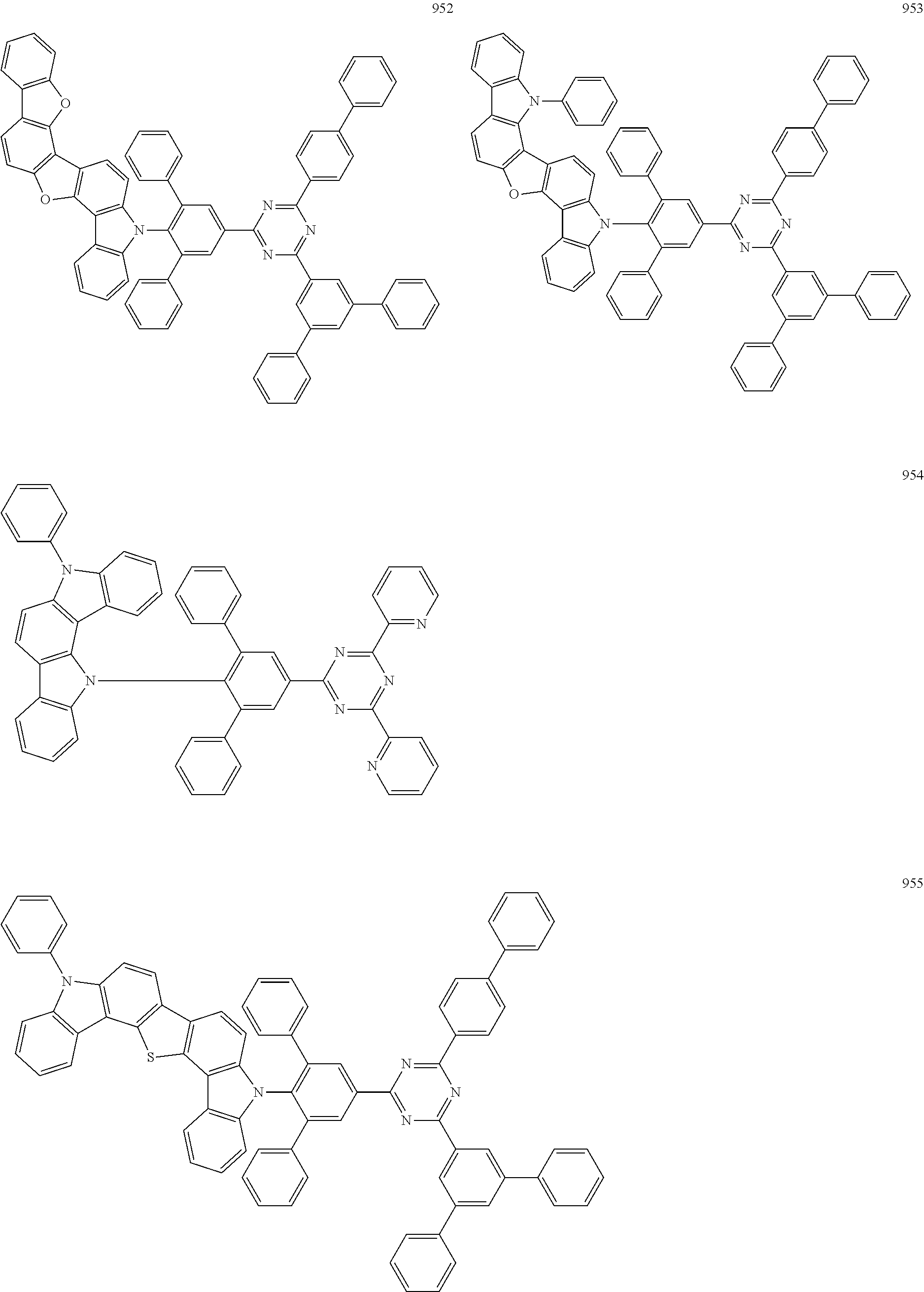

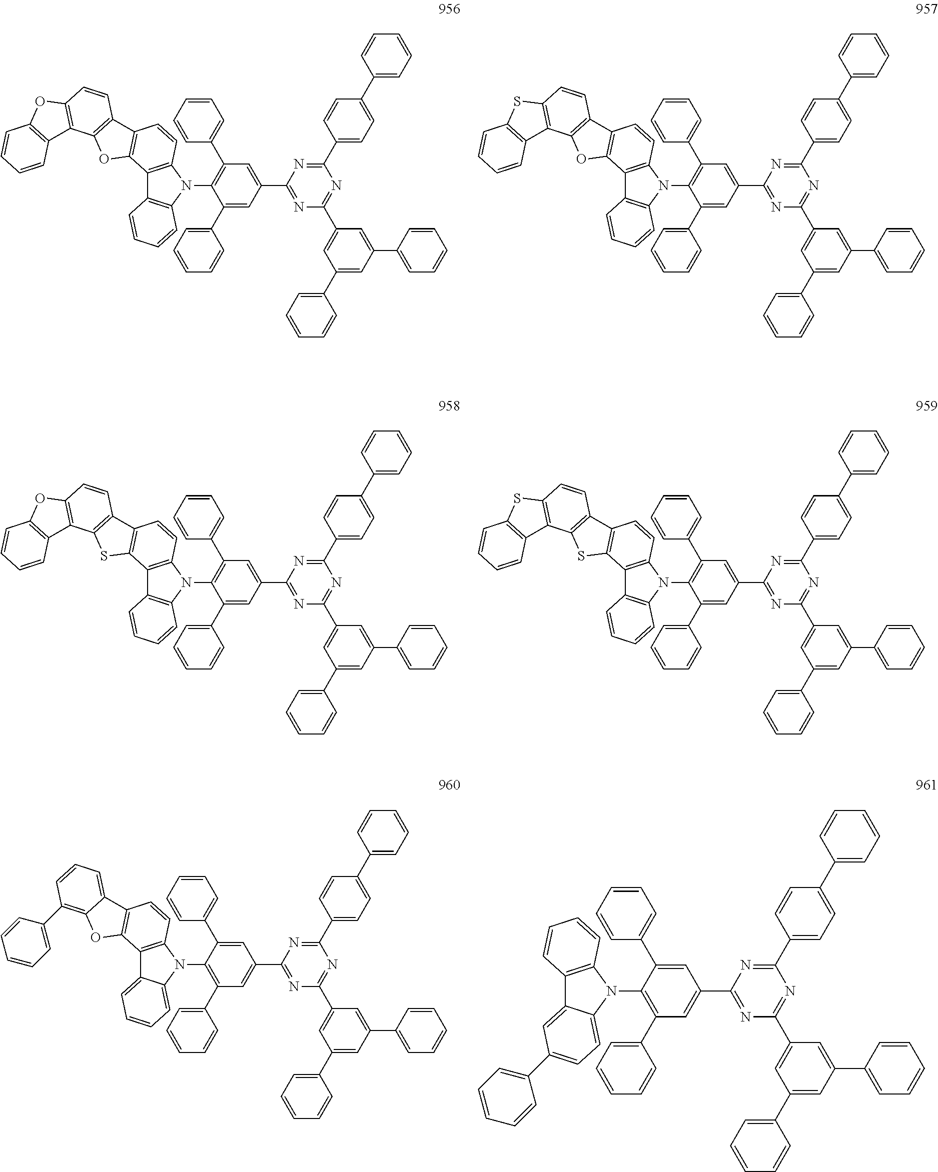

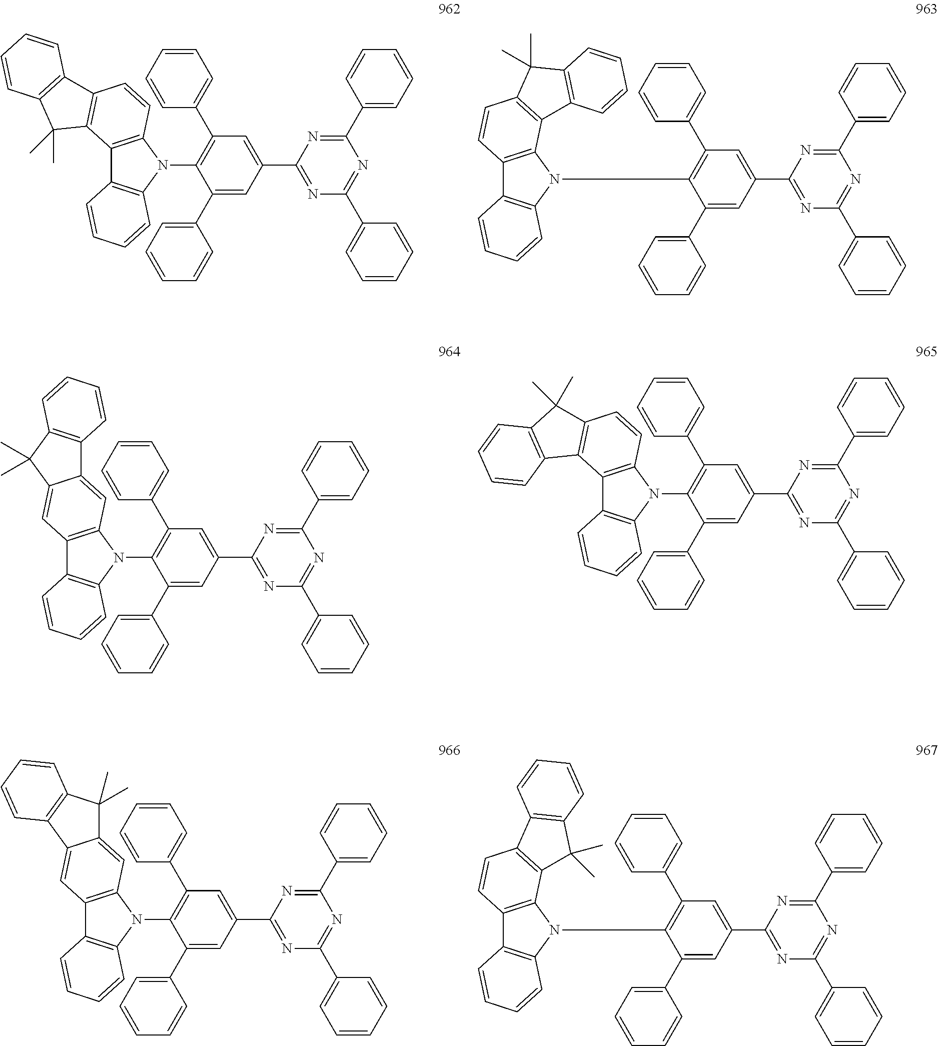

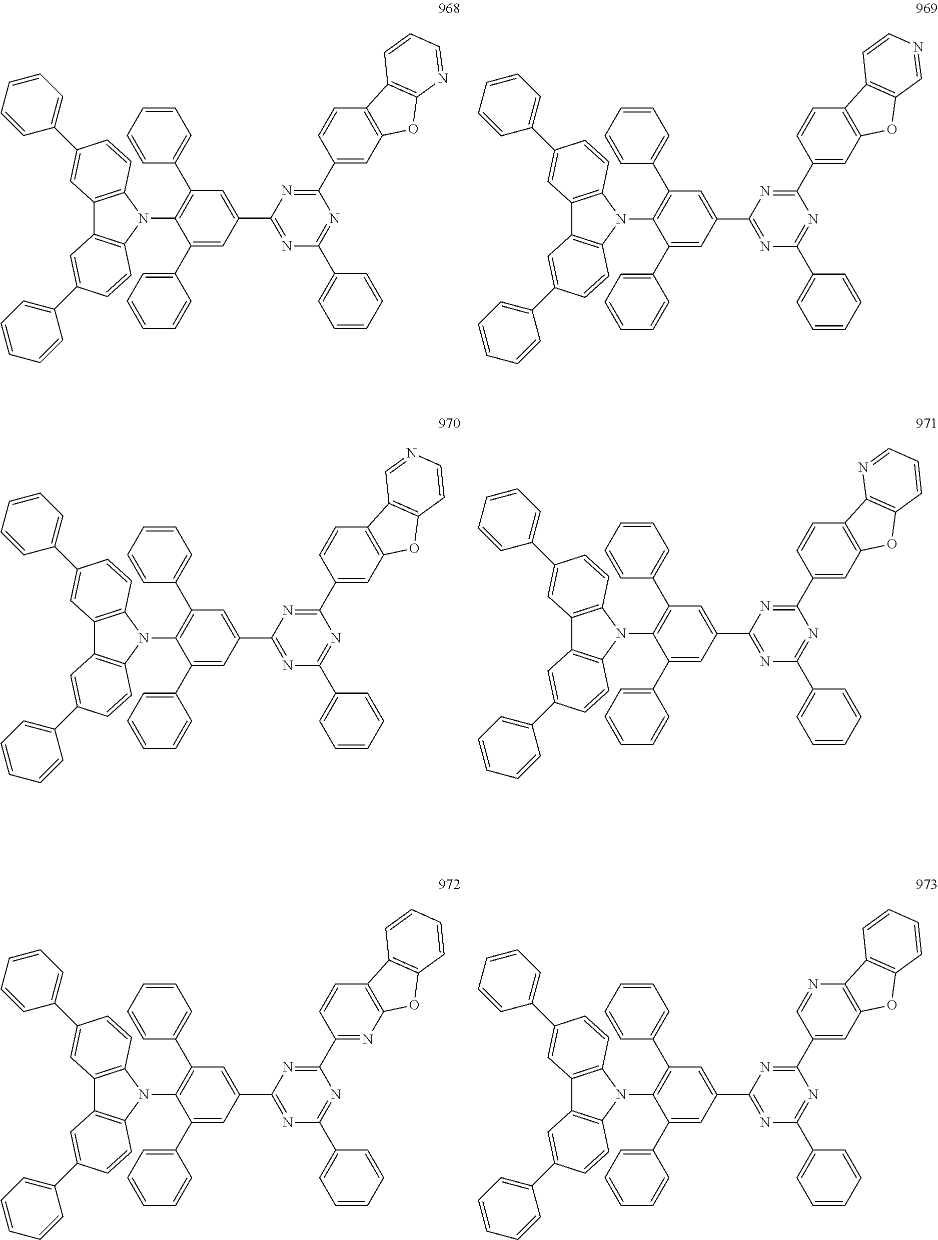

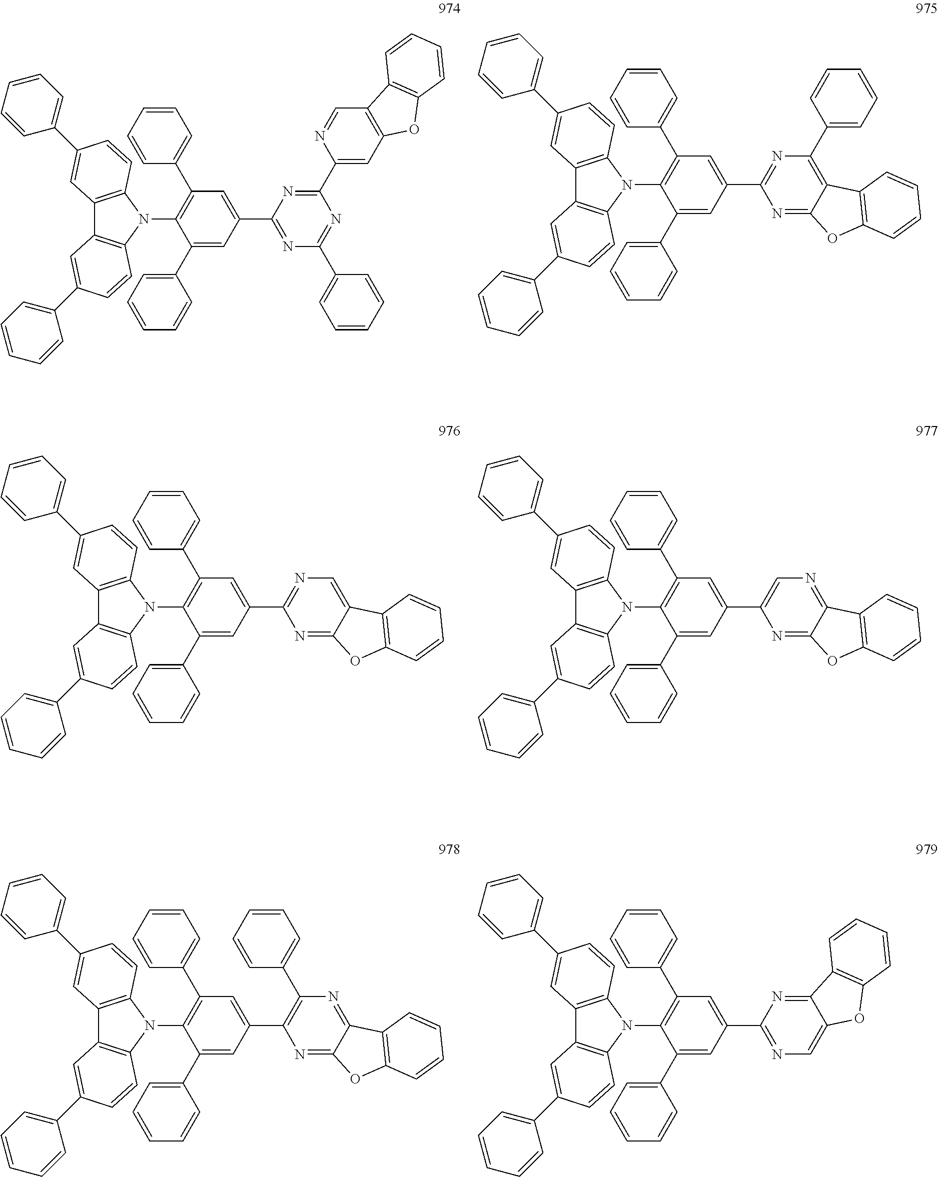

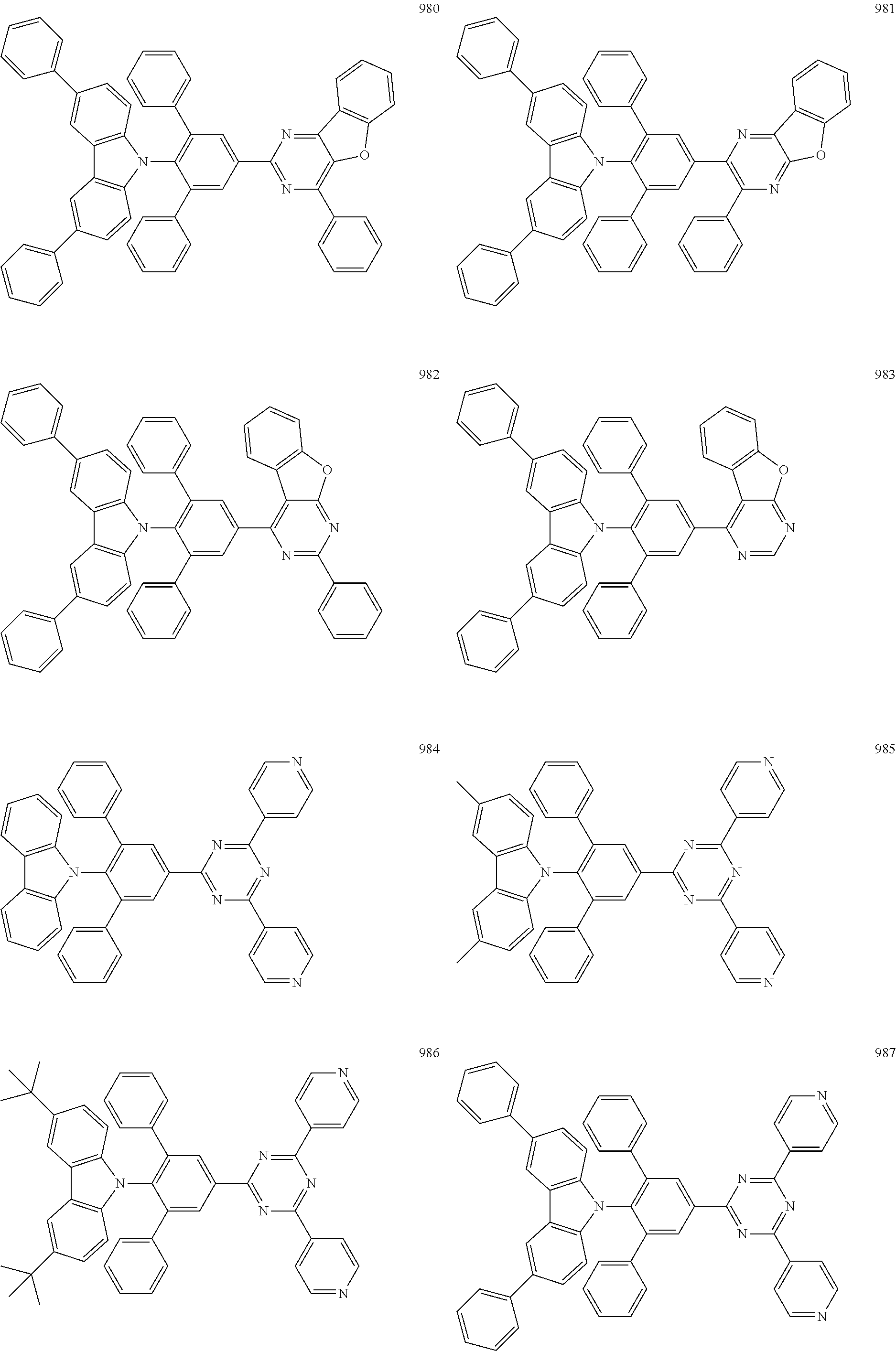

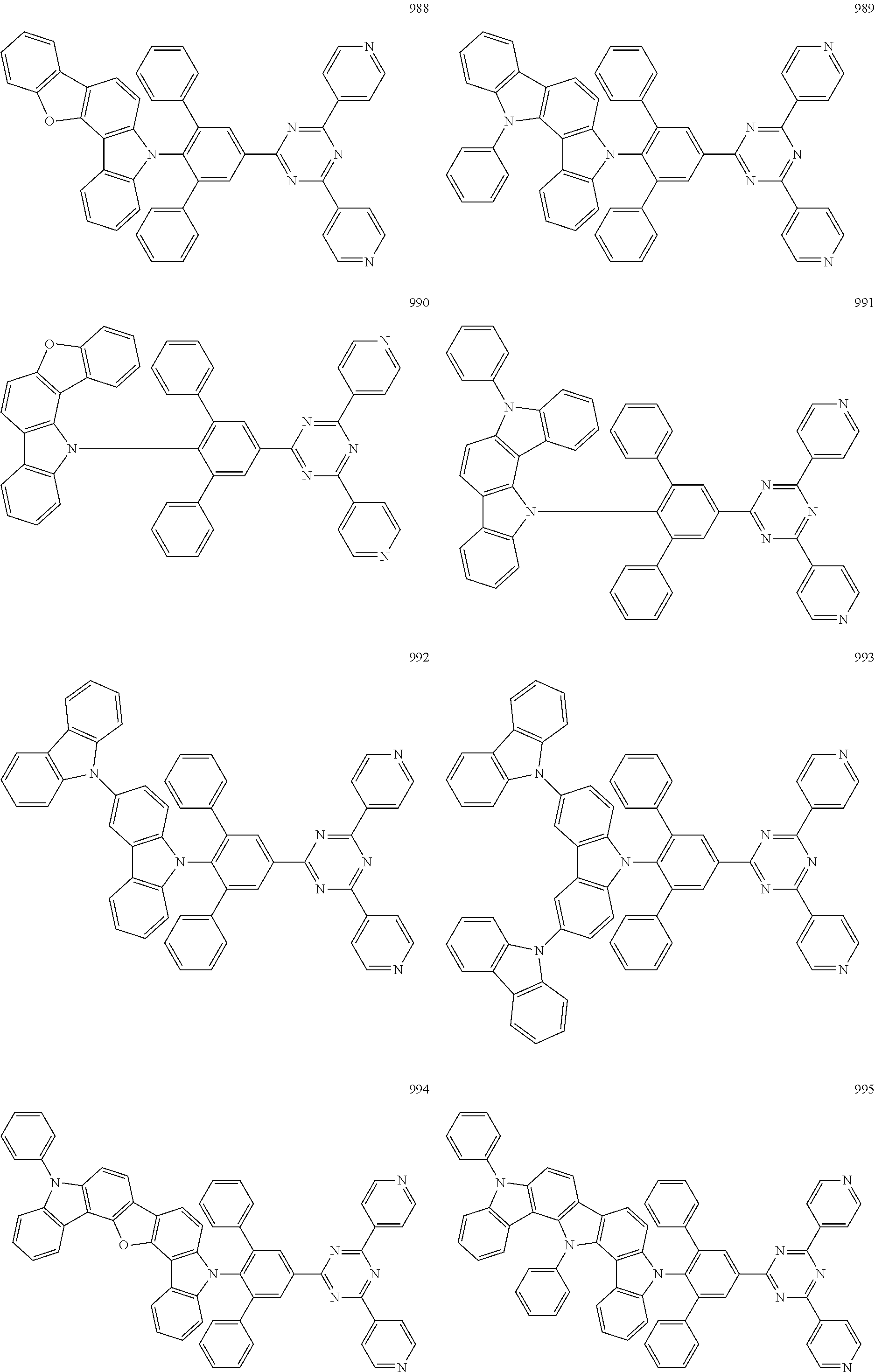

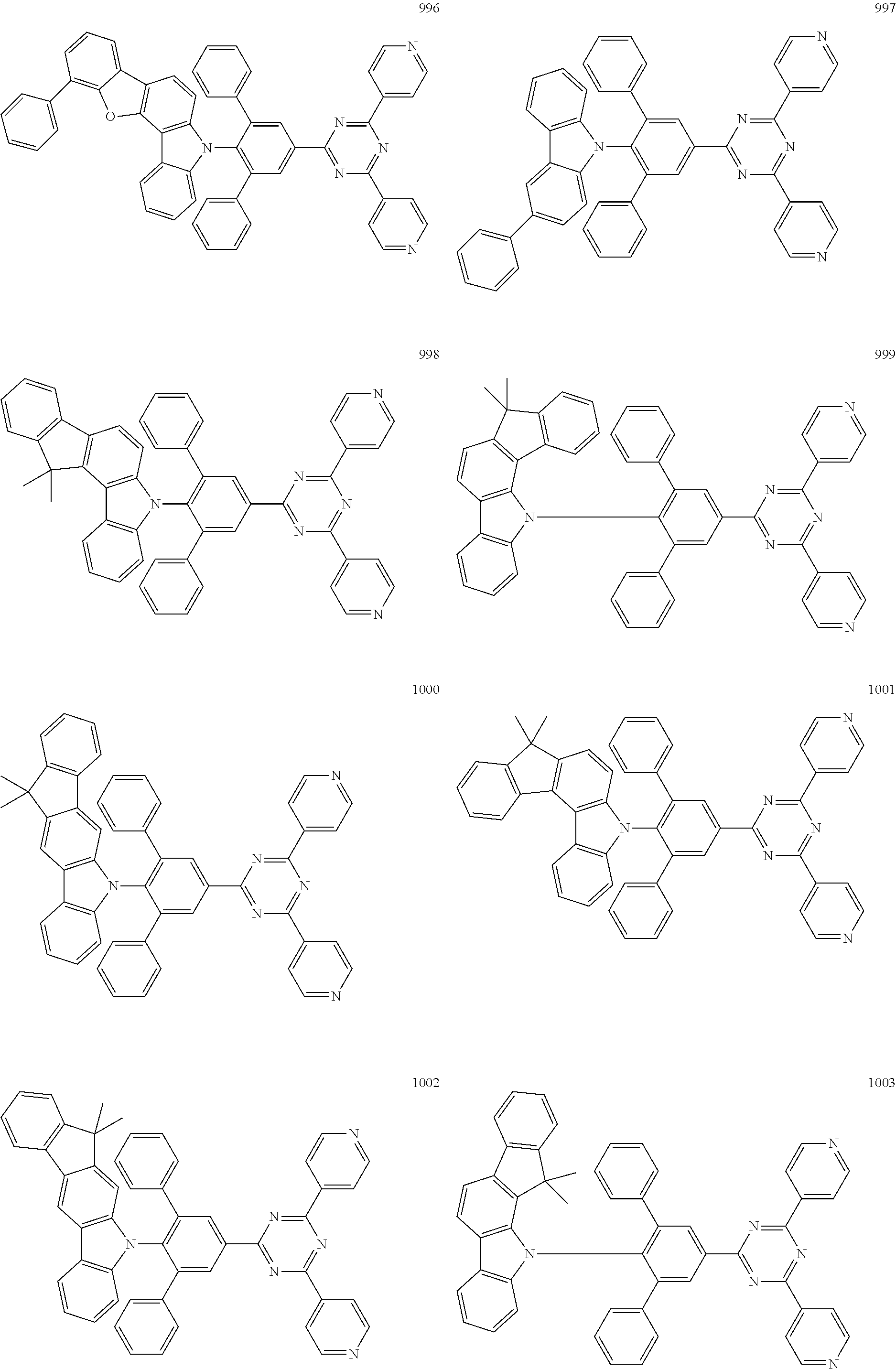

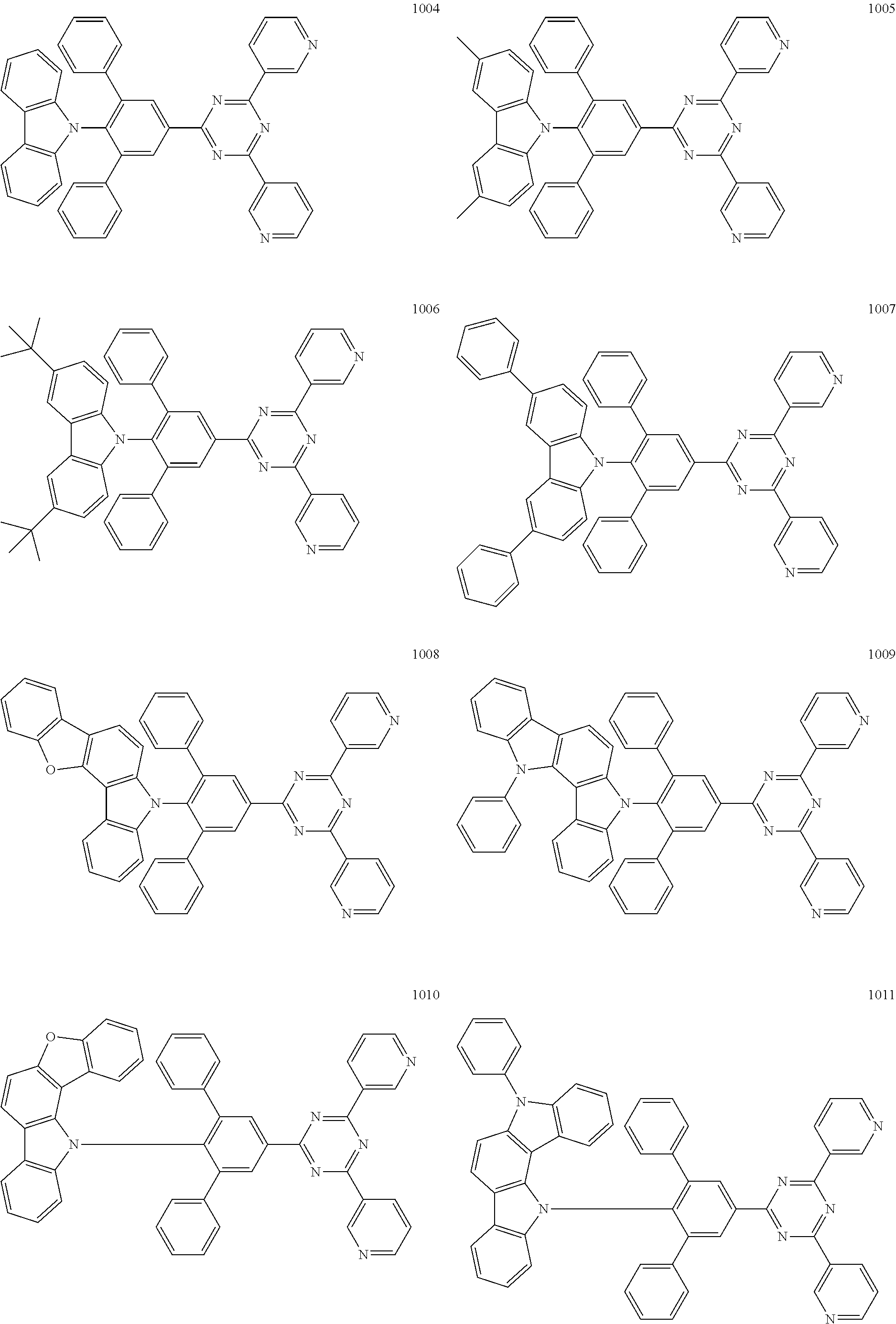

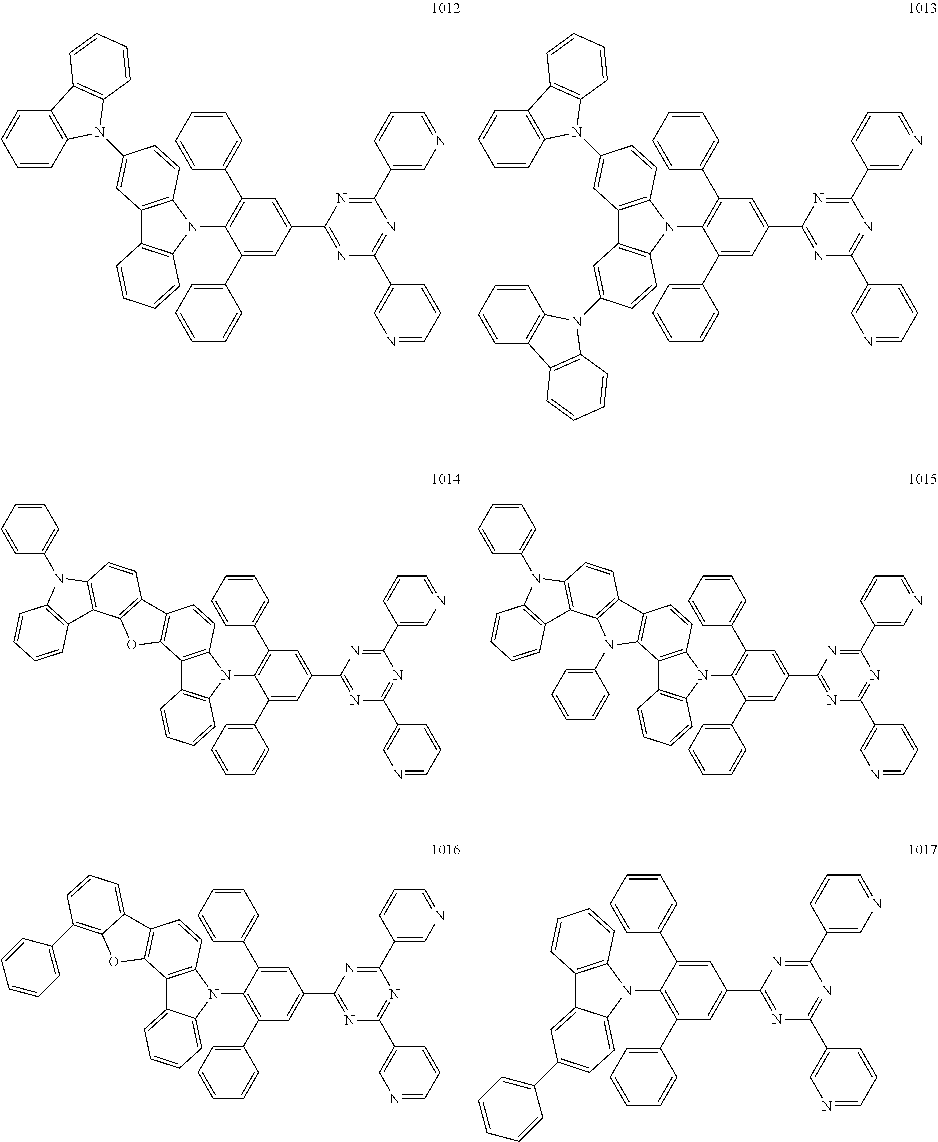

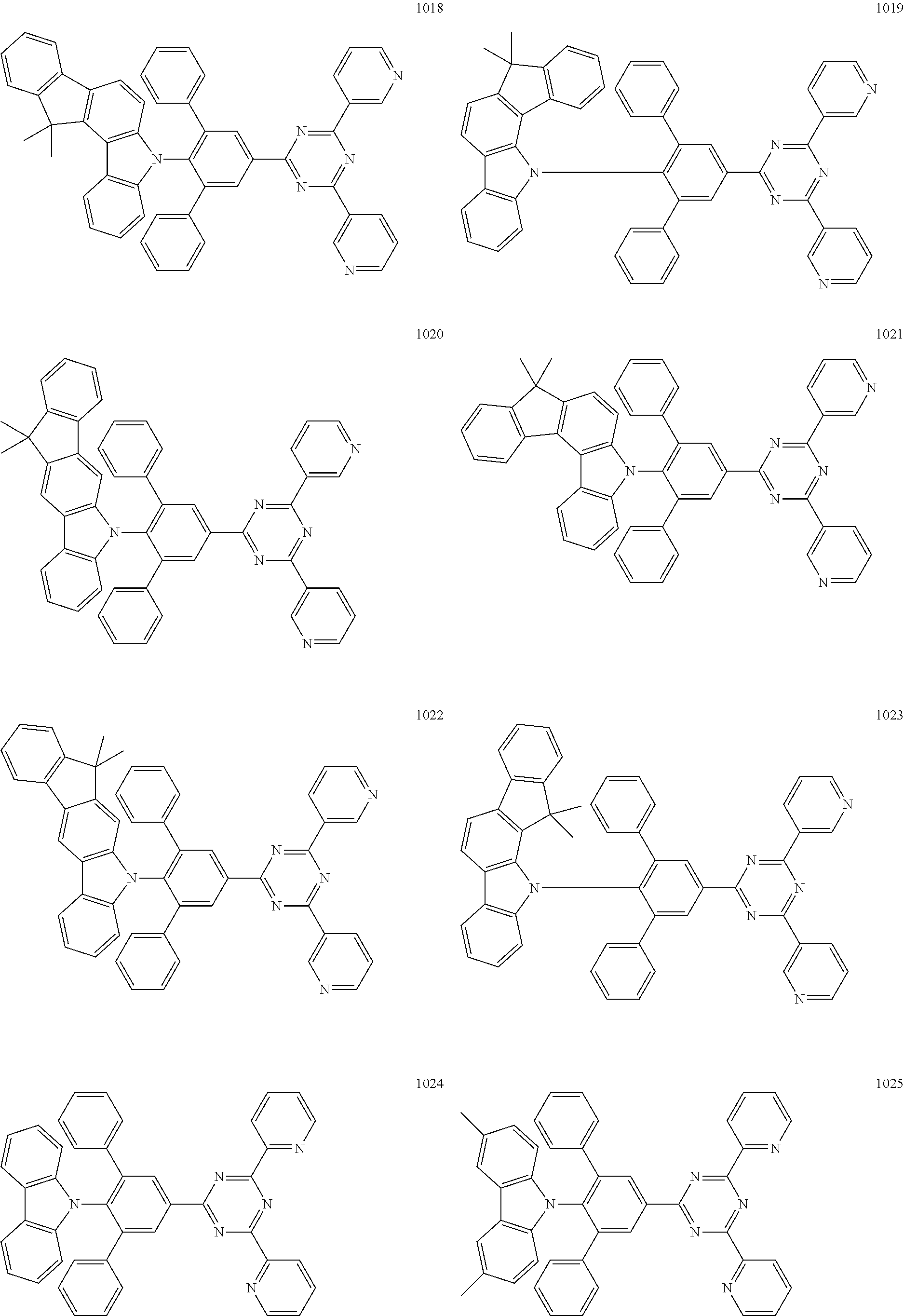

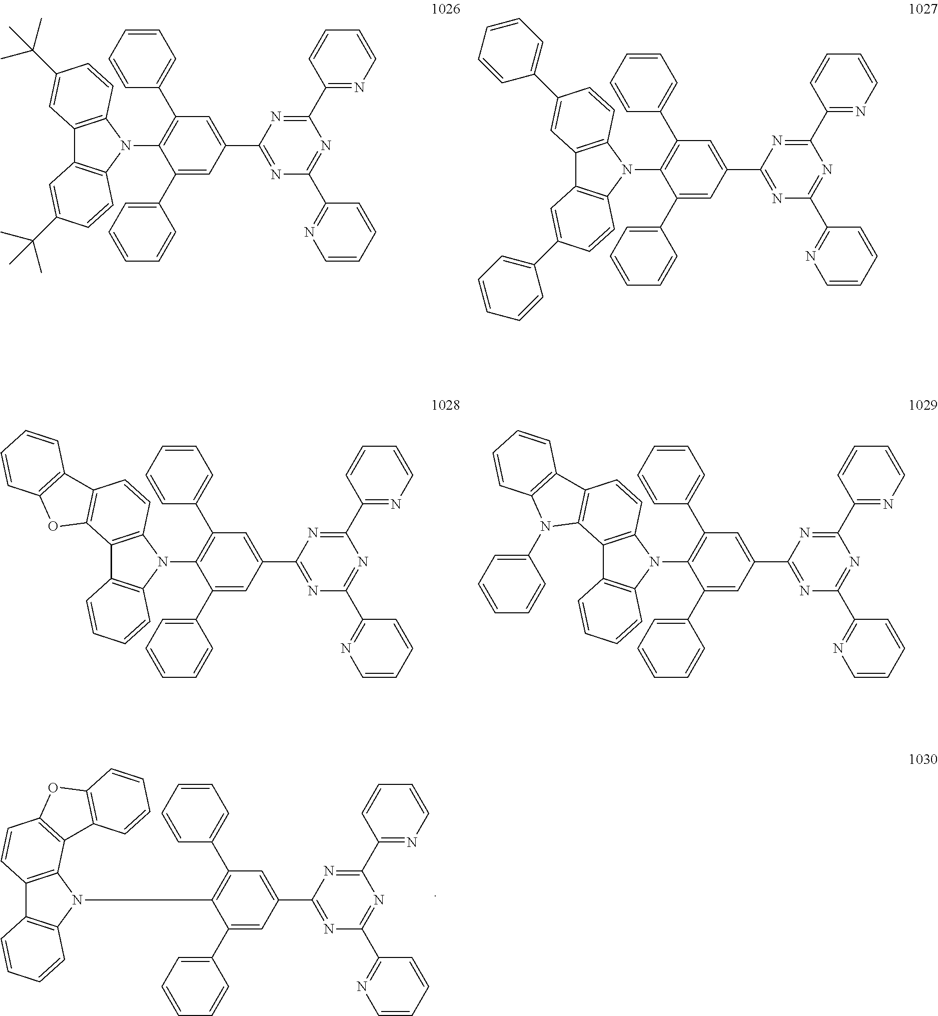

The emission layer including the host and the thermally activated delayed fluorescence emitter as described above does not include a transition metal-containing organometallic compound. That is, the emission layer differs from a phosphorescent emission layer that includes a transition metal-containing organometallic compound and emits phosphorescent light from the transition metal-containing organometallic compound.

The delayed fluorescence component emitted from the thermally activated delayed fluorescence emitter may be about 90% or more, about 92% or more, about 94% or more, about 96% or more, or about 98% or more based on the total emission component emitted from the emission layer including the host and the thermally activated delayed fluorescence emitter.

The emission layer may variously emit red light, green light, and blue light according to the maximum emission wavelength of the thermally activated delayed fluorescence emitter.

In one embodiment, the light emitted from the thermally activated delayed fluorescence emitter in the emission layer may be blue light, but embodiments of the present disclosure are not limited thereto.

An amount of the thermally activated delayed fluorescence emitter in the emission layer may be in a range of about 0.01 parts by weight to about 30 parts by weight based on 100 parts by weight of the host, but embodiments of the present disclosure are not limited thereto. When the amount of the thermally activated delayed fluorescence emitter is within this range, a high-quality organic light-emitting device having no concentration quenching may be obtained.

In one or more embodiments, an amount of the first material in the emission layer may be larger than an amount of the second material, but embodiments of the present disclosure are not limited thereto.

In one embodiment, since the emission layer includes the host and the thermally activated delayed fluorescence emitter but does not include the phosphorescent compound (for example, the transition metal-containing organometallic compound), the organic light-emitting device including the emission layer may emit not phosphorescent light but delayed fluorescent light and may have high efficiency and a long lifespan at the same time.

The FIGURE is a schematic view of an organic light-emitting device 10 according to an embodiment. Hereinafter, the structure of an organic light-emitting device according to an embodiment and a method of manufacturing an organic light-emitting device according to an embodiment will be described in connection with the FIGURE. The organic light-emitting device 10 includes a first electrode 11, an organic layer 15, and a second electrode 19, which are sequentially stacked.

A substrate may be additionally disposed under the first electrode 11 or above the second electrode 19. For use as the substrate, any substrate that is used in general organic light-emitting devices may be used, and the substrate may be a glass substrate or a transparent plastic substrate, each having excellent mechanical strength, thermal stability, transparency, surface smoothness, ease of handling, and water resistance.

The first electrode 11 may be formed by depositing or sputtering a material for forming the first electrode 11 on the substrate. The first electrode 11 may be an anode. The material for forming the first electrode 11 may be materials with a high work function to facilitate hole injection. The first electrode 11 may be a reflective electrode, a semi-transmissive electrode, or a transmissive electrode. The material for forming the first electrode may be, for example, indium tin oxide (ITO), indium zinc oxide (IZO), tin oxide (SnO₂), and zinc oxide (ZnO). In one or more embodiments, magnesium (Mg), aluminum (Al), aluminum-lithium (Al—Li), calcium (Ca), magnesium-indium (Mg—In), or magnesium-silver (Mg—Ag) may be used as the material for forming the first electrode.

The first electrode 11 may have a single-layered structure or a multi-layered structure including two or more layers. For example, the first electrode 11 may have a three-layered structure of ITO/Ag/ITO, but the structure of the first electrode 110 is not limited thereto.

The organic layer 15 is disposed on the first electrode 11.

The organic layer 15 may include a hole transport region, an emission layer, and an electron transport region.

The hole transport region may be disposed between the first electrode 11 and the emission layer.

The hole transport region may include a hole injection layer, a hole transport layer, an electron blocking layer, a buffer layer, or a combination thereof.

The hole transport region may include only either a hole injection layer or a hole transport layer. In one or more embodiments, the hole transport region may have a hole injection layer/hole transport layer structure or a hole injection layer/hole transport layer/electron blocking layer structure, which are sequentially stacked in this stated order from the first electrode 11.

A hole injection layer may be formed on the first electrode 11 by using one or more suitable methods selected from vacuum deposition, spin coating, casting, or Langmuir-Blodgett (LB) deposition.

When a hole injection layer is formed by vacuum deposition, the deposition conditions may vary according to a compound that is used to form the hole injection layer, and the structure and thermal characteristics of the hole injection layer. For example, the deposition conditions may include a deposition temperature of about 100° C. to about 500° C., a vacuum pressure of about 10⁻⁸ torr to about 10⁻³ torr, and a deposition rate of about 0.01 A/sec to about 100 Aisec. However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 rpm to about 5,000 rpm, and a temperature at which a heat treatment is performed to remove a solvent after coating may be from about 80° C. to about 200° C. However, the coating conditions are not limited thereto.

Conditions for forming a hole transport layer and an electron blocking layer may be understood by referring to conditions for forming the hole injection layer.

The hole transport region may include m-MTDATA, TDATA, 2-TNATA, NPB, 3-NPB, TPD, Spiro-TPD, Spiro-NPB, methylated-NPB, TAPC, HMTPD, 4,4′, 4″-tris(N-carbazolyl)triphenylamine (TCTA), polyaniline/dodecylbenzenesulfonic acid (PAN I/DBSA), poly(3,4-ethylenedioxythiophene)/poly(4-styrenesulfonate) (PEDOT/PSS), polyaniline/camphor sulfonic acid (PANI/CSA), polyaniline/poly(4-styrene sulfonate) (PANI/PSS), a compound represented by Formula 201 below, a compound represented by Formula 202 below, or a combination thereof:

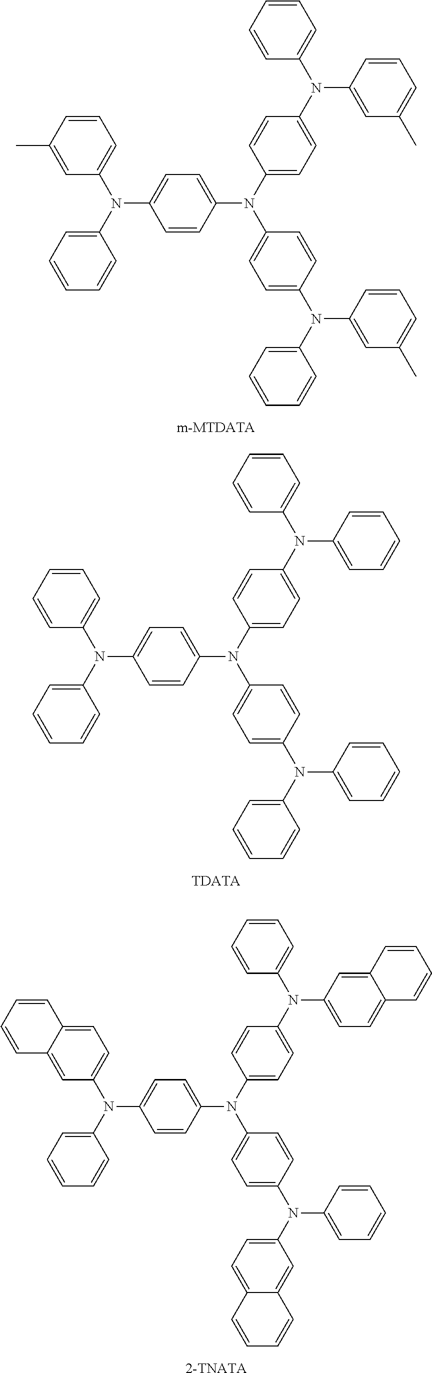

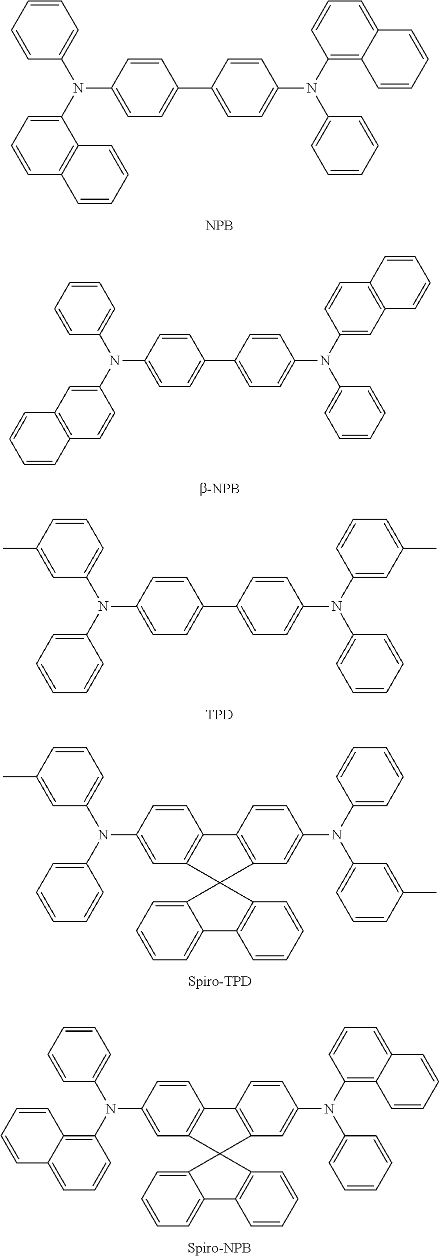

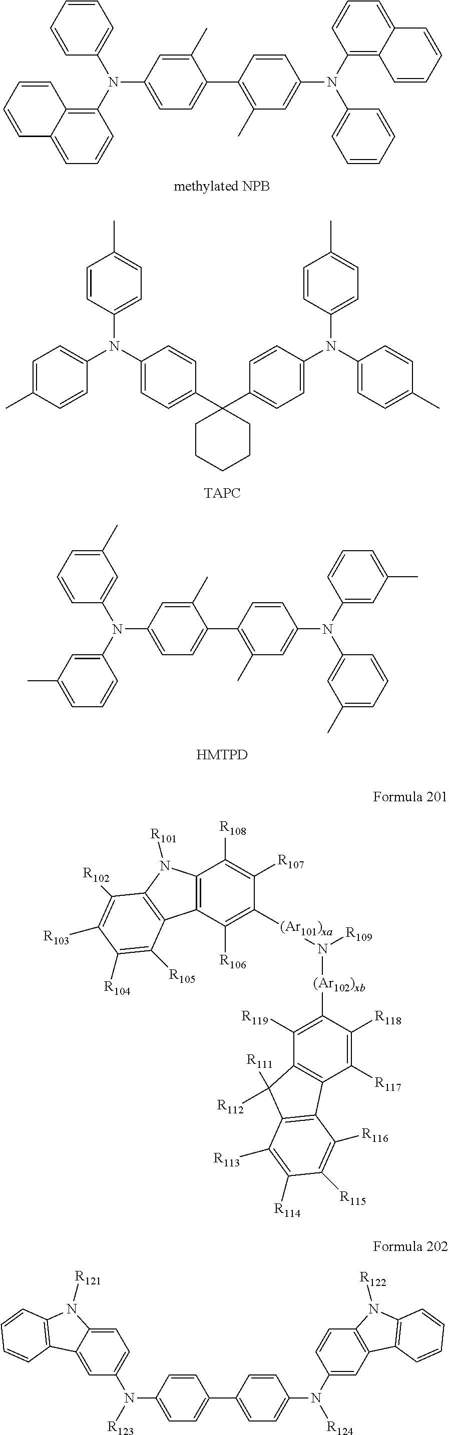

Ar₁₀₁ and Ar₁₀₂ in Formula 201 may each independently be:

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group; or

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₂-C₁₀ heterocycloalkyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, or any combination thereof.

In Formula 201, xa and xb may each independently be an integer from 0 to 5, or 0, 1, or 2. For example, xa is 1 and xb is 0, but xa and xb are not limited thereto.

R₁₀₁ to R₁₀₈, R₁₁₁ to R₁₁₉, and R₁₂₁ to R₁₂₄ in Formulae 201 and 202 may each independently be:

a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, and a C₁-C₁₀ alkyl group (for example, a methyl group, an ethyl group, a propyl group, a butyl group, a pentyl group, a hexyl group, and so on), or a C₁-C₁₀ alkoxy group (for example, a methoxy group, an ethoxy group, a propoxy group, a butoxy group, a pentoxy group, and the like);

a C₁-C₁₀ alkyl group or a C₁-C₁₀ alkoxy group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, or a combination thereof;

a phenyl group, a naphthyl group, an anthracenyl group, a fluorenyl group, or a pyrenyl group; or

a phenyl group, a naphthyl group, an anthracenyl group, a fluorenyl group, or a pyrenyl group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, or a combination thereof, but embodiments of the present disclosure are not limited thereto.

R₁₀₉ in Formula 201 may be:

a phenyl group, a naphthyl group, an anthracenyl group, or a pyridinyl group; or a phenyl group, a naphthyl group, an anthracenyl group, or a pyridinyl group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, a pyridinyl group, or a combination thereof.

According to an embodiment, the compound represented by Formula 201 may be represented by Formula 201A, but embodiments of the present disclosure are not limited thereto:

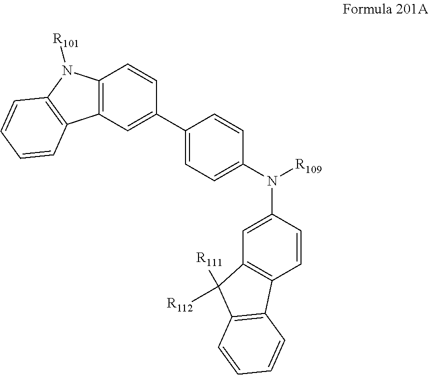

R₁₀₁, R₁₁₁, R₁₁₂, and R₁₀₉ in Formula 201A may each independently have the same definition as described above.

For example, the compound represented by Formula 201, and the compound represented by Formula 202 may include compounds HT1 to HT20 illustrated below, but are not limited thereto.

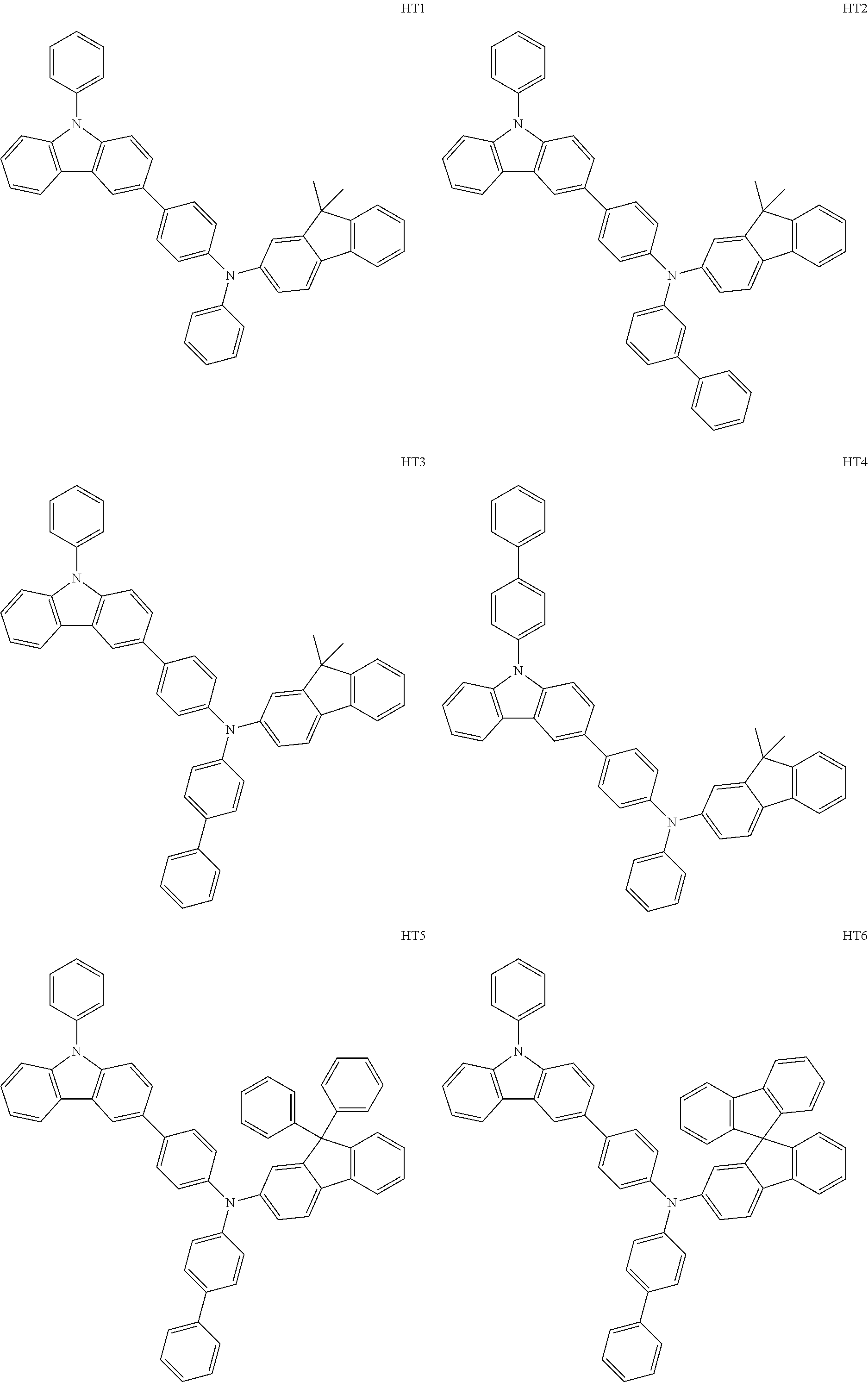

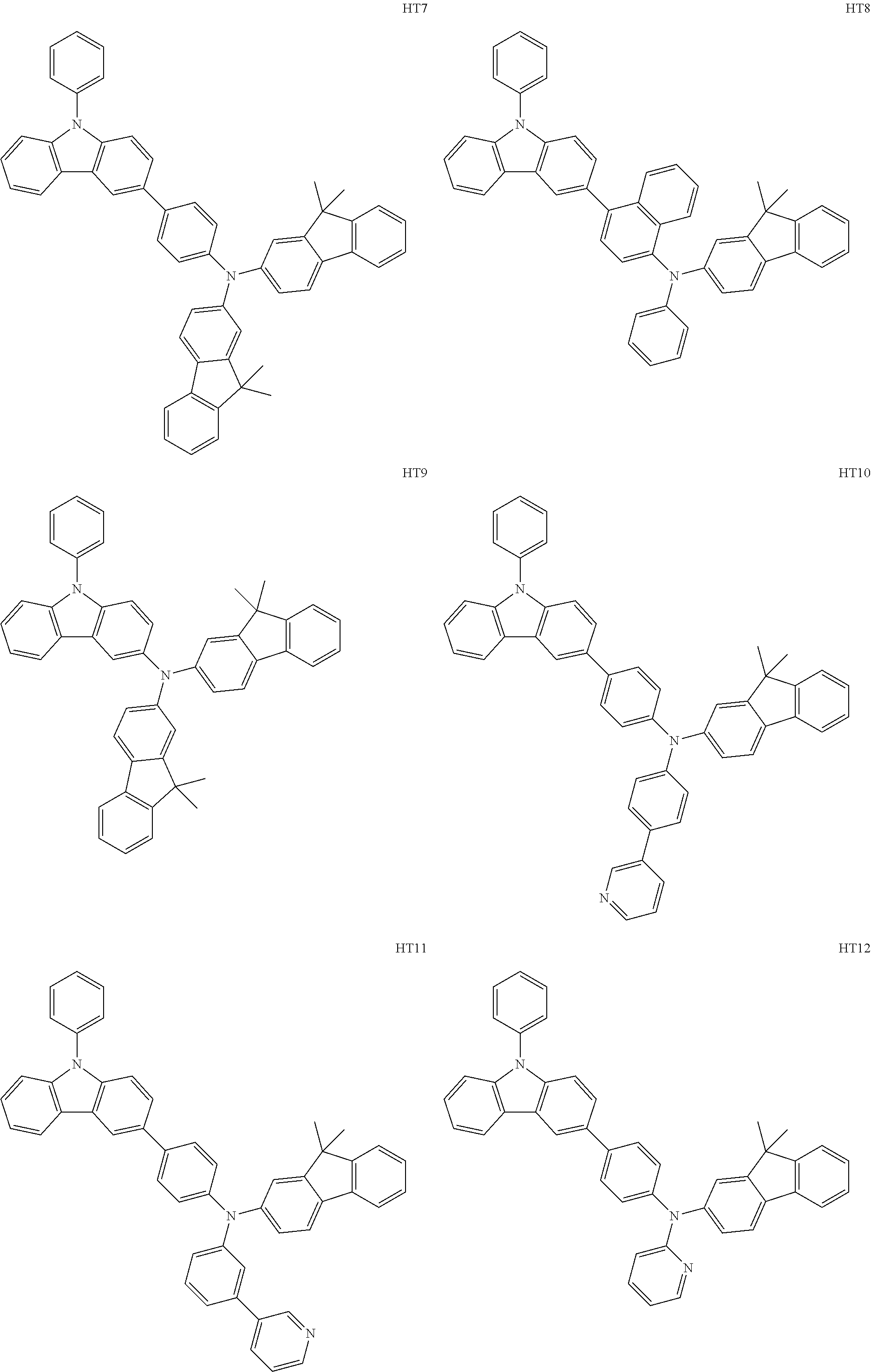

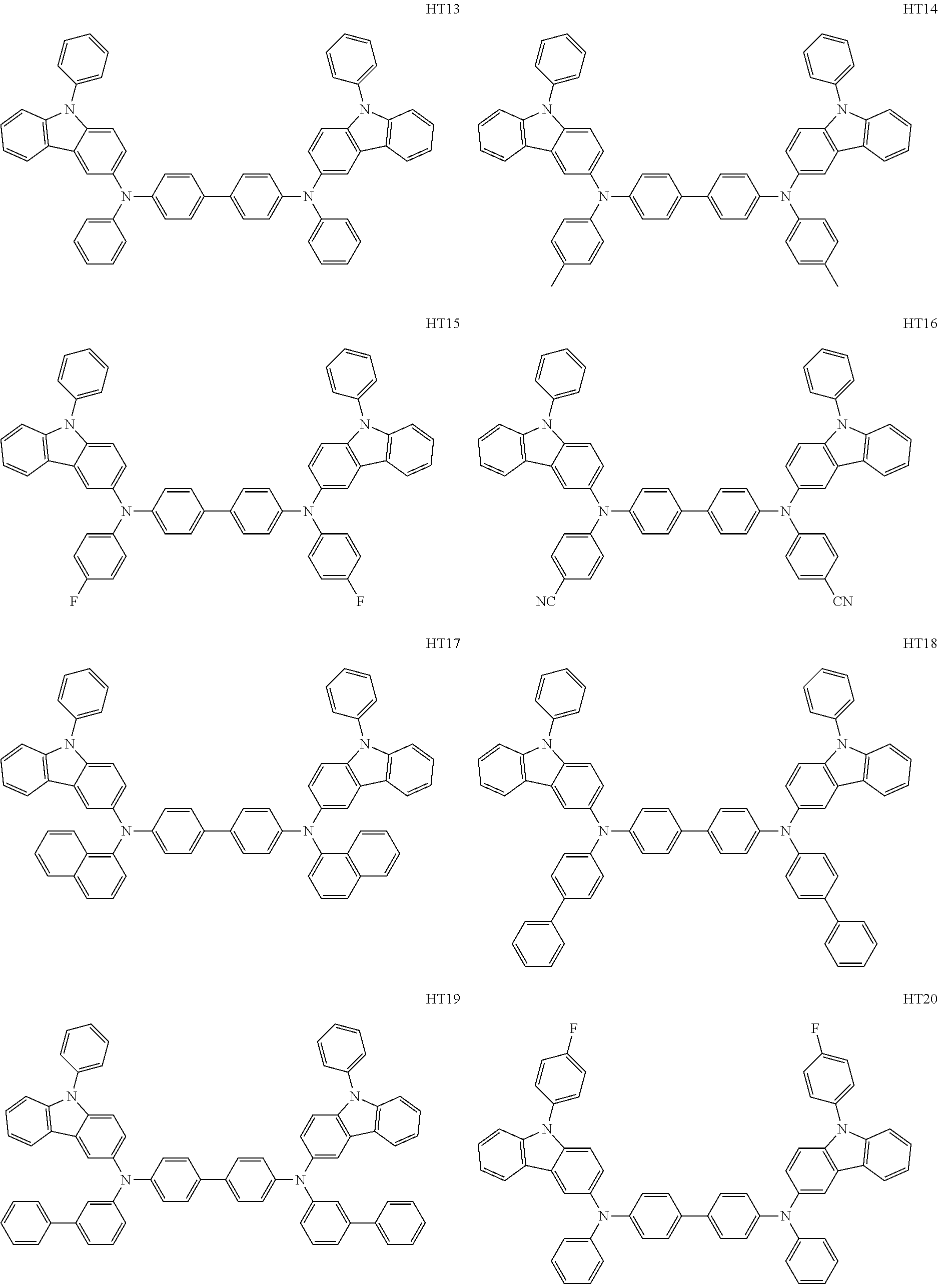

A thickness of the hole transport region may be in a range of about 100 Å to about 10,000 Å, for example, about 100 Å to about 1,000 Å. When the hole transport region includes a hole injection layer and a hole transport layer, the thickness of the hole injection layer may be in a range of about 100 Å to about 10,000 Å, and for example, about 100 Å to about 1,000 Å, and the thickness of the hole transport layer may be in a range of about 50 Å to about 2,000 Å, and for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be a quinone derivative, a metal oxide, or a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenium oxide; and a cyano group-containing compound, such as Compound HT-D1 or Compound HT-D2 below, but are not limited thereto.

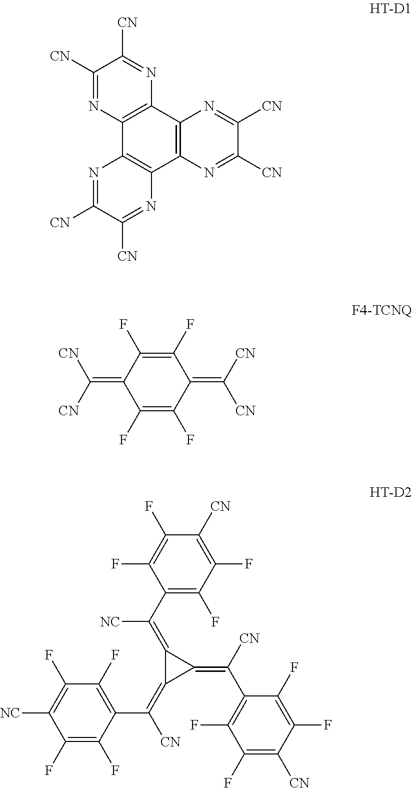

The hole transport region may include a buffer layer.

Also, the buffer layer may compensate for an optical resonance distance according to a wavelength of light emitted from the emission layer, and thus, efficiency of a formed organic light-emitting device may be improved.

The electron transport region may further include an electron blocking layer. The electron blocking layer may include, for example, mCP, but a material therefor is not limited thereto.

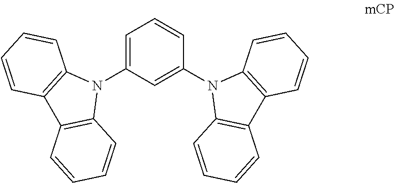 [SMILES: c1cc(-n2c3ccccc3c3ccccc32)cc(-n2c3ccccc3c3ccccc32)c1]

Then, an emission layer may be formed on the hole transport region by vacuum deposition, spin coating, casting, LB deposition, or the like. When the emission layer is formed by vacuum deposition or spin coating, the deposition or coating conditions may be similar to those applied in forming the hole injection layer although the deposition or coating conditions may vary according to a compound that is used to form the emission layer.

When the organic light-emitting device is a full-color organic light-emitting device, the emission layer may be patterned into a red emission layer, a green emission layer, and a blue emission layer. In one or more embodiments, due to a stacked structure including a red emission layer, a green emission layer, and/or a blue emission layer, the emission layer may emit white light.

The emission layer may include a host and a thermally activated delayed fluorescence emitter, and the host and the thermally activated delayed fluorescence emitter may be the same as described above.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

Then, an electron transport region may be disposed on the emission layer.

The electron transport region may include a hole blocking layer, an electron transport layer, an electron injection layer, or a combination thereof.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, BCP, Bphen, or a combination thereof, but may also include other materials.

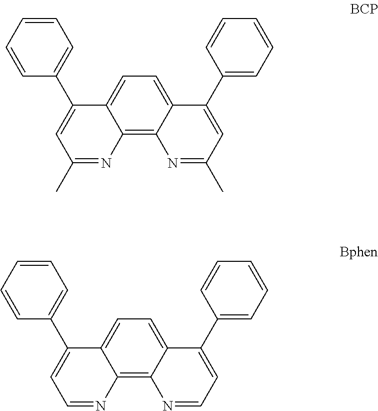 [SMILES: Cc1cc(-c2ccccc2)c2ccc3c(-c4ccccc4)cc(C)nc3c2n1 ; c1ccc(-c2ccnc3c2ccc2c(-c4ccccc4)ccnc23)cc1]

A thickness of the hole blocking layer may be in a range of about 20 Å to about 1,000 Å, for example, about 30 Å to about 300 Å. When the thickness of the hole blocking layer is within these ranges, the hole blocking layer may have improved hole blocking ability without a substantial increase in driving voltage.

The electron transport layer may further include BCP, Bphen, Alq₃, BAlq, TAZ, NTAZ, or a combination thereof.

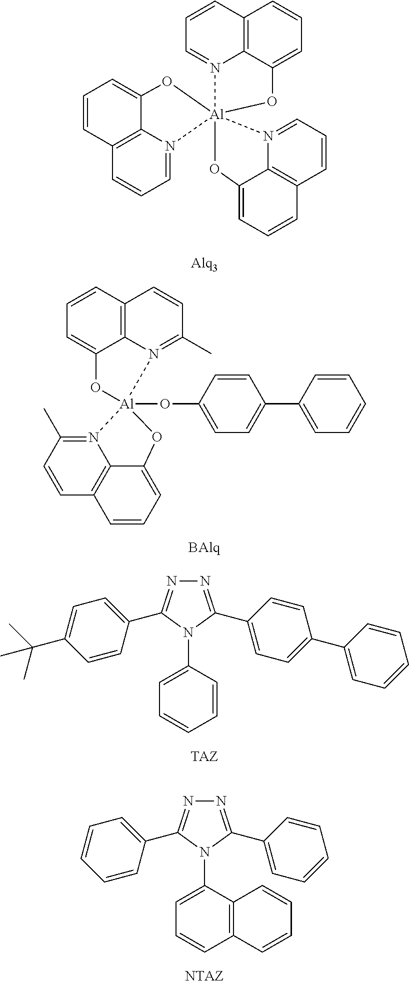

In one or more embodiments, the electron transport layer may include ET1 to ET25, but are not limited thereto:

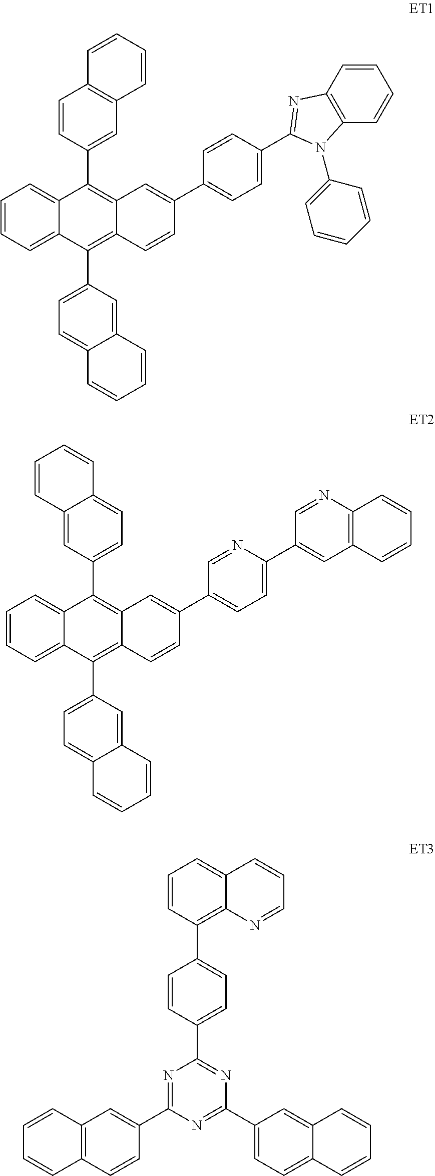 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

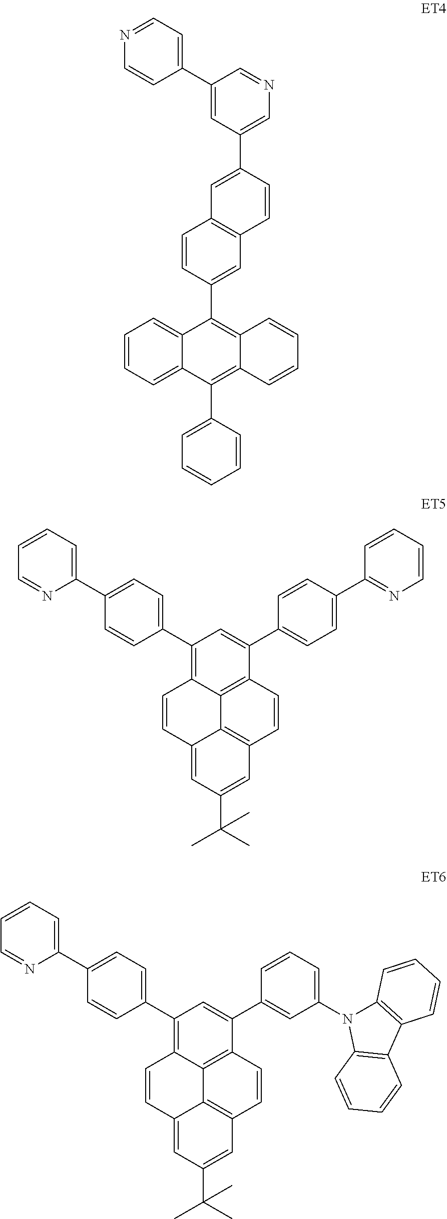 [SMILES: c1ccc(-c2c3ccccc3c(-c3ccc4cc(-c5cncc(-c6ccncc6)c5)ccc4c3)c3ccccc23)cc1 ; CC(C)(C)c1cc2ccc3c(-c4ccc(-c5ccccn5)cc4)cc(-c4ccc(-c5ccccn5)cc4)c4ccc(c1)c2c34 ; CC(C)(C)c1cc2ccc3c(-c4ccc(-c5ccccn5)cc4)cc(-c4cccc(-n5c6ccccc6c6ccccc65)c4)c4ccc(c1)c2c34]

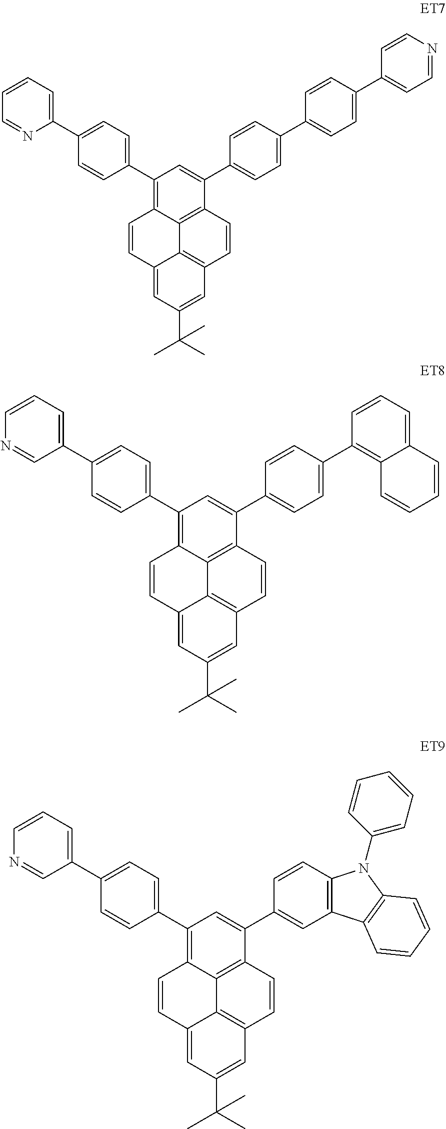 [SMILES: CC(C)(C)c1cc2ccc3c(-c4ccc(-c5ccc(-c6ccncc6)cc5)cc4)cc(-c4ccc(-c5ccccn5)cc4)c4ccc(c1)c2c34 ; CC(C)(C)c1cc2ccc3c(-c4ccc(-c5cccnc5)cc4)cc(-c4ccc(-c5cccc6ccccc56)cc4)c4ccc(c1)c2c34 ; CC(C)(C)c1cc2ccc3c(-c4ccc(-c5cccnc5)cc4)cc(-c4ccc5c(c4)c4ccccc4n5-c4ccccc4)c4ccc(c1)c2c34]

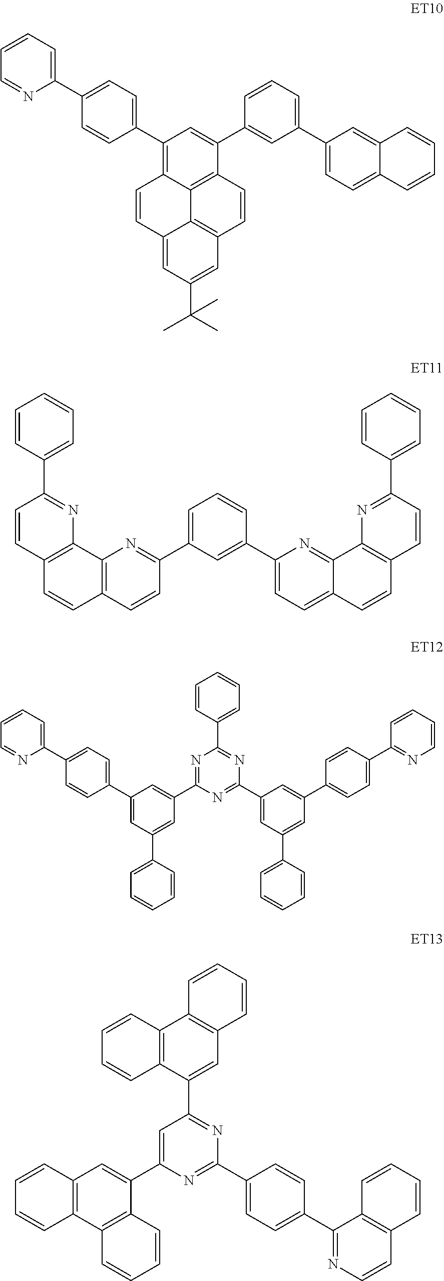 [SMILES: CC(C)(C)c1cc2ccc3c(-c4ccc(-c5ccccn5)cc4)cc(-c4cccc(-c5ccc6ccccc6c5)c4)c4ccc(c1)c2c34 ; c1ccc(-c2ccc3ccc4ccc(-c5cccc(-c6ccc7ccc8ccc(-c9ccccc9)nc8c7n6)c5)nc4c3n2)cc1 ; c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1 ; c1ccc2c(-c3ccc(-c4nc(-c5cc6ccccc6c6ccccc56)cc(-c5cc6ccccc6c6ccccc56)n4)cc3)nccc2c1]

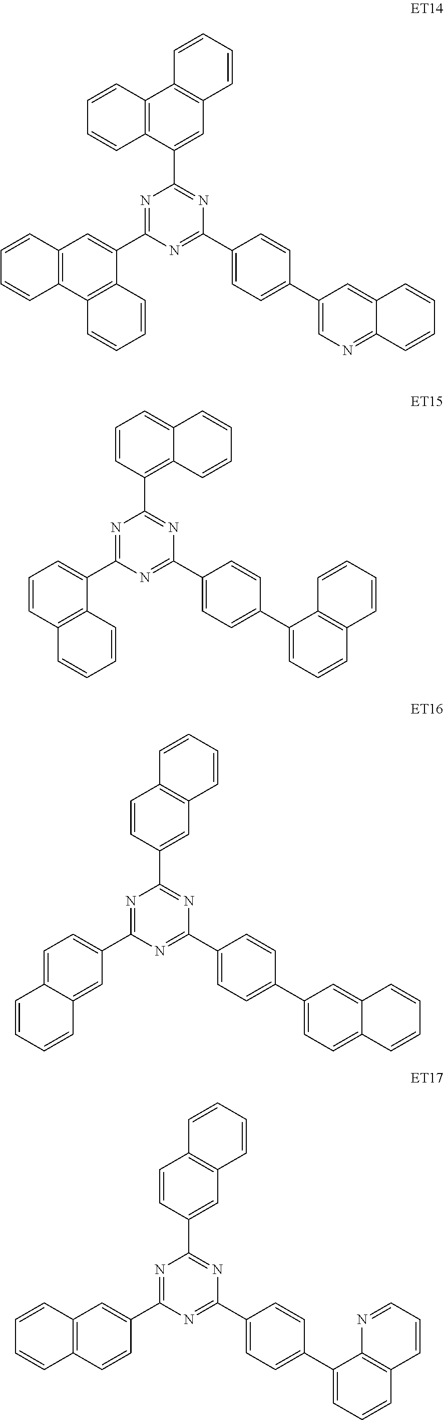 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc2c(-c3ccc(-c4nc(-c5cccc6ccccc56)nc(-c5cccc6ccccc56)n4)cc3)cccc2c1 ; c1ccc2cc(-c3ccc(-c4nc(-c5ccc6ccccc6c5)nc(-c5ccc6ccccc6c5)n4)cc3)ccc2c1 ; c1ccc2cc(-c3nc(-c4ccc(-c5cccc6cccnc56)cc4)nc(-c4ccc5ccccc5c4)n3)ccc2c1]

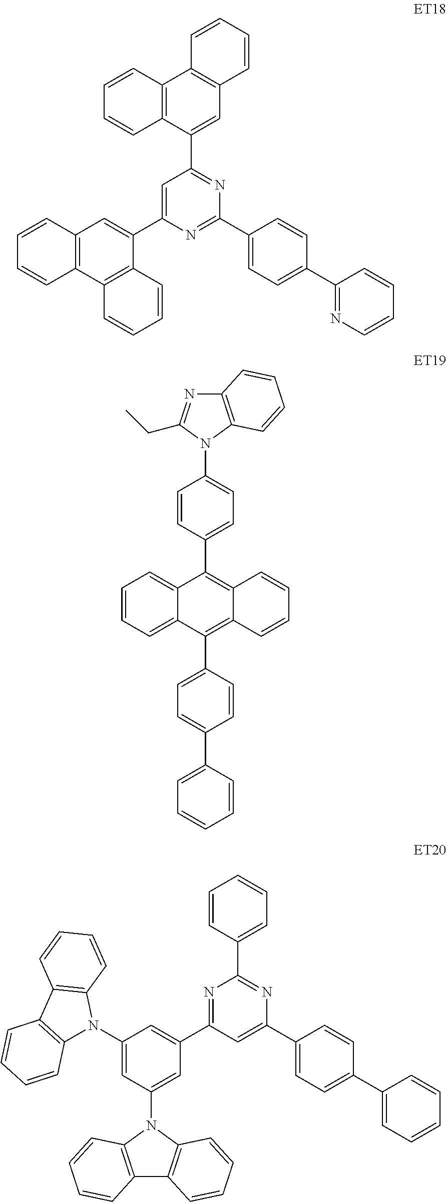 [SMILES: c1ccc(-c2ccc(-c3nc(-c4cc5ccccc5c5ccccc45)cc(-c4cc5ccccc5c5ccccc45)n3)cc2)nc1 ; CCc1nc2ccccc2n1-c1ccc(-c2c3ccccc3c(-c3ccc(-c4ccccc4)cc3)c3ccccc23)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

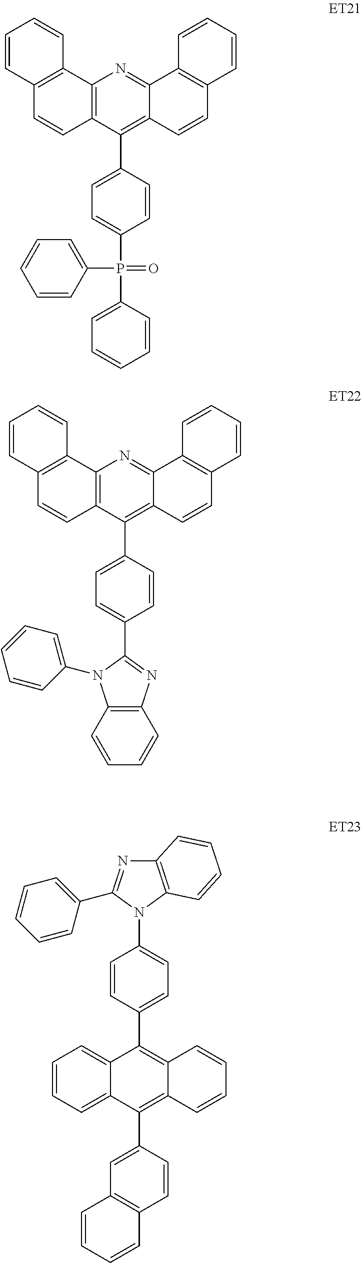 [SMILES: O=P(c1ccccc1)(c1ccccc1)c1ccc(-c2c3ccc4ccccc4c3nc3c2ccc2ccccc23)cc1 ; c1ccc(-n2c(-c3ccc(-c4c5ccc6ccccc6c5nc5c4ccc4ccccc45)cc3)nc3ccccc32)cc1 ; c1ccc(-c2nc3ccccc3n2-c2ccc(-c3c4ccccc4c(-c4ccc5ccccc5c4)c4ccccc34)cc2)cc1]

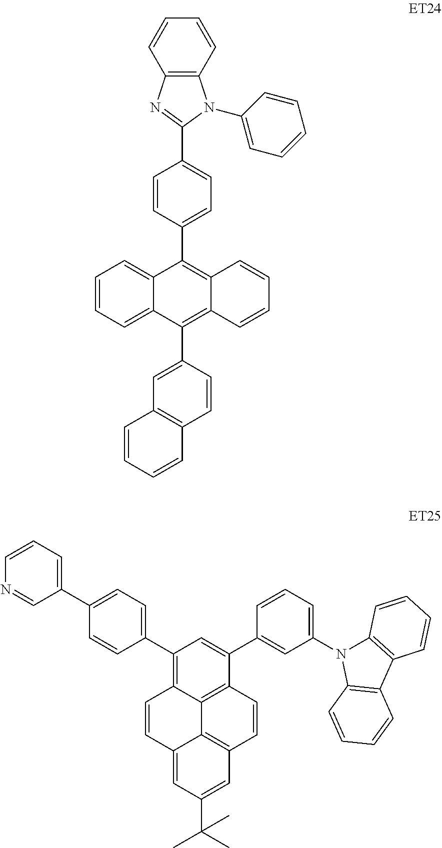 [SMILES: c1ccc(-n2c(-c3ccc(-c4c5ccccc5c(-c5ccc6ccccc6c5)c5ccccc45)cc3)nc3ccccc32)cc1 ; CC(C)(C)c1cc2ccc3c(-c4ccc(-c5cccnc5)cc4)cc(-c4cccc(-n5c6ccccc6c6ccccc65)c4)c4ccc(c1)c2c34]

A thickness of the electron transport layer may be in a range of about 100 Å to about 1,000 Å, for example, about 150 Å to about 500 Å. When the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a Li complex. The Li complex may include, for example, Compound ET-D1 (lithium 8-hydroxyquinolate, LiQ) or ET-D2.

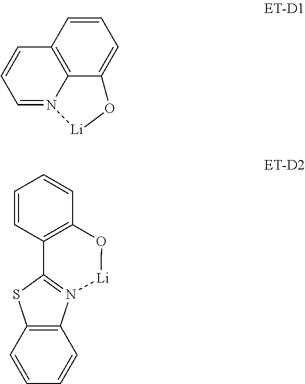

The electron transport region may include an electron injection layer (EIL) that promotes flow of electrons from the second electrode 19 thereinto.

The electron injection layer may include LiF, NaCl, CsF, Li₂O, BaO, or a combination thereof.

A thickness of the electron injection layer may be in a range of about 1 Å to about 100 Å, for example, about 3 Å to about 90 Å. When the thickness of the electron injection layer is within the range described above, the electron injection layer may have satisfactory electron injection characteristics without a substantial increase in driving voltage.

The second electrode 19 is disposed on the organic layer 15. The second electrode 19 may be a cathode. A material for forming the second electrode 19 may be a metal, an alloy, an electrically conductive compound, or a combination thereof, which have a relatively low work function. For example, lithium (Li), magnesium (Mg), aluminum (Al), aluminum-lithium (A-Li), calcium (Ca), magnesium-indium (Mg—In), or magnesium-silver (Mg-Ag) may be used as a material for forming the second electrode 19. In one or more embodiments, to manufacture a top-emission type light-emitting device, a transmissive electrode formed using ITO or IZO may be used as the second electrode 19.

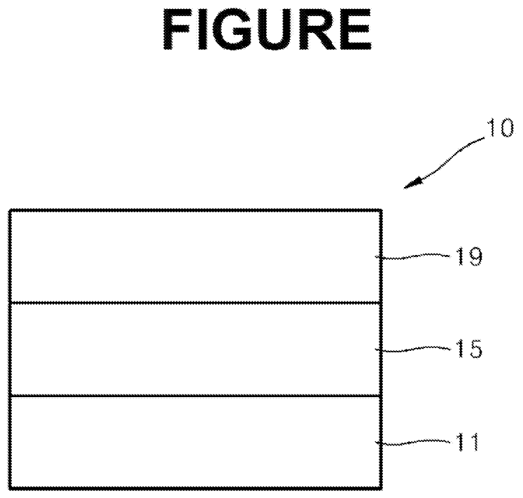

Hereinbefore, the organic light-emitting device has been described with reference to FIG. 1, but embodiments of the present disclosure are not limited thereto.

The term “C₁-C₆₀ alkyl group” as used herein refers to a linear or branched saturated aliphatic hydrocarbon monovalent group having 1 to 60 carbon atoms, and non-limiting examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is the C₁-C₆₀ alkyl group), and non-limiting examples thereof include a methoxy group, an ethoxy group, and an isopropyloxy group.

The term “C₂-C₆₀ alkenyl group” as used herein refers to a hydrocarbon group formed by substituting at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocydic group having 3 to 10 carbon atoms, and non-limiting examples thereof include a cydopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₂-C₁₀ heterocycloalkyl group” as used herein refers to a monovalent saturated monocyclic group having at least one heteroatom selected from N, O, P, Si and S as a ring-forming atom and 2 to 10 carbon atoms, and non-limiting examples thereof include a tetrahydrofuranyl group, and a tetrahydrothiophenyl group. The term “C₂-C₁₀ heterocycloalkylene group” as used herein refers to a divalent group having the same structure as the C₂-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₂-C₁₀ heterocycloalkenyl group” as used herein refers to a monovalent monocyclic group that has at least one heteroatom selected from N, O, P, Si, and S as a ring-forming atom, 2 to 10 carbon atoms, and at least one carbon-carbon double bond in its ring. Non-limiting examples of the C₂-C₁₀ heterocycloalkenyl group include a 2,3-dihydrofuranyl group, and a 2,3-dihydrothiophenyl group. The term “C₂-C₁₀ heterocycloalkenylene group” as used herein refers to a divalent group having the same structure as the C₂-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₂-C₆₀ heteroaryl group” as used herein refers to a monovalent group having a heterocyclic aromatic system that has at least one heteroatom selected from N, O, P, Si, and S as a ring-forming atom, and 2 to 60 carbon atoms. The term “C₂-C₆₀ heteroarylene group,” as used herein refers to a divalent group having a heterocyclic aromatic system that has at least one heteroatom selected from N, O, P, Si, and S as a ring-forming atom, and 2 to 60 carbon atoms. Non-limiting examples of the C₂-C₆₀ heteroaryl group include a pyridinyl group, a pyrimidinyl group, a pyrazinyl group, a pyridazinyl group, a triazinyl group, a quinolinyl group, and an isoquinolinyl group. When the C₆-C₆₀ heteroaryl group and the C₂-C₆₀ heteroarylene group each include two or more rings, the rings may be fused to each other.

The term “C₆-C₆₀ aryloxy group” as used herein refers to —OA₁₀₂ (wherein A₁₀₂ is the C₆-C₆₀ aryl group), and a C₆-C₆₀ arylthio group used herein indicates —SA₁₀₃ (wherein A₁₀₃ is the C₆-C₆₀ aryl group).

The term “monovalent non-aromatic condensed polycyclic group” as used herein refers to a monovalent group having two or more rings condensed to each other, only carbon atoms (for example, the number of carbon atoms may be in a range of 8 to 60) as a ring-forming atom, and no aromaticity in its entire molecular structure. Non-limiting examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group having two or more rings condensed to each other, a heteroatom selected from N, O, P, Si, and S, other than carbon atoms (for example, the number of carbon atoms may be in a range of 2 to 60), as a ring-forming atom, and no aromaticity in its entire molecular structure. Non-limiting examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

At least one substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic condensed heteropolycyclic group may be:

a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;

a C₁-C₆₀ alkyl group, a C₂-C₆₀ alkenyl group, a C₂-C₆₀ alkynyl group, or a C₁-C₆₀ alkoxy group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), —B(Q₁₆)(Q₁₇), or a combination thereof;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, or a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, or a monovalent non-aromatic condensed heteropolycyclic group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), —B(Q₂)(Q₂₇), or a combination thereof; or

—Si(Q₃₁)(Q₃₂)(Q₃₃), —N(Q₃₄)(Q₃₅), or —B(Q₃₆)(Q₃₇), and Q₁ to Q₇, Q₁ to Q₁₇, Q₂₁ to Q₂₇, and Q₃₁ to Q₃₇ may each independently be a hydrogen, a substituted or unsubstituted C₁-C₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, or a substituted or unsubstituted monovalent non-aromatic condensed heteropolycyclic group.

The term “room temperature” as used herein refers to about 25° C.

The term “a biphenyl group and a terphenyl group” as used herein refers to a monovalent group in which two or three benzene groups are linked via a single bond.

Hereinafter, a compound and an organic light-emitting device according to embodiments are described in detail with reference to Synthesis Example and Examples. However, the organic light-emitting device is not limited thereto. The wording “B was used instead of A” used in describing Synthesis Examples means that an amount of A used was identical to an amount of B used, in terms of a molar equivalent.

### EXAMPLES

### Synthesis Example 1: Synthesis of Compound 4

Compound 4 was synthesized according to the Reaction Scheme below.

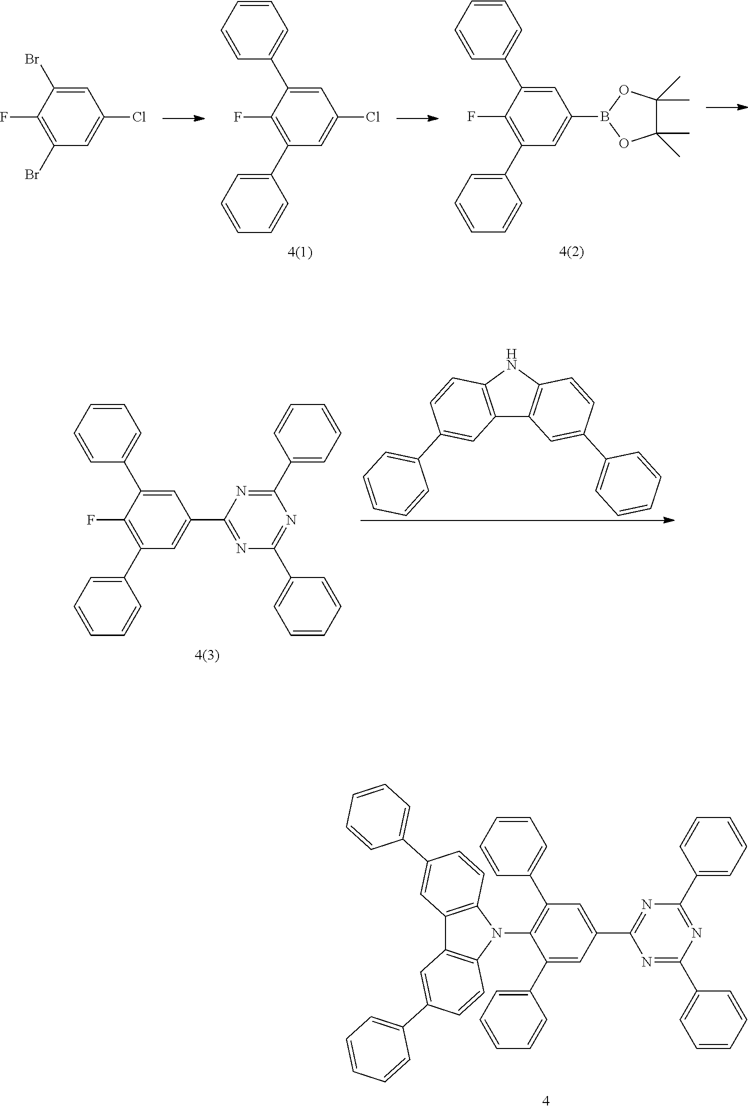

### Synthesis of Intermediate 4(1)

Phenylboronic acid (63.43 g, 520.22 mmol), 1,3-dibromo-5-chloro-2-fluorobenzene (50 g, 173.41 mmol), palladium tetrakis(triphenylphosphine) (Pd(PPh₃)4) (20.04 g, 17.34 mmol), potassium carbonate (K₂CO₃) (95.87 g, 693.63 mmol), and S-phos (14.24 g, 34.68 mmol) were added to 300 milliliters (ml) of tetrahydrofuran and 300 ml of distilled water and heated under reflux. After the reaction was completed, the reaction product was cooled to room temperature, and an organic layer was extracted therefrom by using ethyl acetate, dried by anhydrous sodium sulfate (Na₂SO₄), concentrated, and then separated by silica gel column chromatography (dichloromethane/hexane). A solid obtained therefrom was recrystallized by using hexane to obtain Intermediate 4(1) (40.7 g, 143.81 mmol, yield of 83%) that was a white solid.

### Synthesis of Intermediate 4(2)

Intermediate 4(1) (40.7 g, 143.81 mmol), bis(pinacolato)diboron (54.78 g, 215.71 mmol), potassium acetate (35.29 g, 359.52 mmol), tris(dibenzylideneacetone)dipalladium(0) (Pd₂(dba)₃) (13.17 g, 14.38 mmol), and tricyclohexylphosphine (4.03 g, 14.38 mmol) were added to 290 ml of dioxane and heated under reflux. After the reaction was completed, the reaction product was cooled to room temperature, dissolved in a large amount of toluene, and filtered through silica gel. An organic layer obtained therefrom was concentrated, precipitated by pouring hexane thereto, and filtered to obtain Intermediate 4(2) (47.0 g, 125.58 mmol, yield of 87%) that was a white solid.

### Synthesis of Intermediate 4(3)

2-chloro-4,6-diphenyl-1,3,5-triazine (18 g, 67.23 mmol), Intermediate 3(2) (30.2 g, 80.68 mmol), palladium tetrakis(triphenylphosphine) (Pd(PPh₃)₄) (3.89 g, 3.36 mmol), potassium carbonate (K₂CO₃) (18.59 g, 134.47 mmol), and S-phos (5.52 g, 13.45 mmol) were added to 120 ml of tetrahydrofuran and 120 ml of distilled water and heated under reflux. After the reaction was completed, the reaction product was cooled to room temperature, and methanol was added thereto. The reaction product was filtered through silica gel. An organic layer obtained therefrom was concentrated and precipitated by pouring methanol thereto to synchronize Intermediate 4(3) (30.0 g, 62.56 mmol, yield of 93%) that was a white solid.

### Synthesis of Compound 4

Intermediate 4(3) (4.80 g, 10 mmol), 3,6-diphenyl-9H-carbazole (15 mmol), and cesium carbonate (Cs₂CO₃) (6.52 g, 20 mmol) were added to 20 ml of N,N-dimethylformamide and stirred at a temperature of 165° C. for 4 hours. After the reaction was completed, the reaction product was cooled to room temperature, and methanol was added thereto. The reaction product was filtered through silica gel. An organic layer obtained therefrom was concentrated, dissolved in toluene, filtered through silica gel, and concentrated. A resulting product was recrystallized (dichloromethane) to synchronize Compound 4 (yield of 68%).

LC-Mass (Calcd. exact mass: 778.31 g/mol, Found: 779.32 g/mol (M+1))

### Evaluation Example 1: Measure of Dipole Moment

The electrostatic potential fitting (ESP) charge and interatomic distance of each atom of Compounds E1, E6 to E45, A, and B were calculated by using a DFT method of Jaguar program (structurally optimized at a level of B3LYP, 6-31G(d,p)), and the dipole moment of the corresponding compound was calculated. Results thereof are shown in Table 1.

TABLE 1

| Compound No. | Dipole moment (debye) |
|---|---|
| E1 | 6.843 |
| E6 | 13.1045 |
| E7 | 11.7574 |
| E8 | 9.1307 |
| E9 | 6.338 |
| E10 | 10.5922 |
| E11 | 8.1047 |
| E12 | 7.2769 |
| E13 | 11.0295 |
| E14 | 8.0784 |
| E15 | 8.7137 |
| E16 | 7.7583 |
| E17 | 6.7516 |
| E18 | 7.3982 |
| E19 | 10.399 |
| E20 | 6.5935 |
| E21 | 8.9665 |
| E22 | 8.9962 |
| E23 | 7.5728 |
| E24 | 8.4826 |
| E25 | 8.4548 |
| E26 | 7.7327 |
| E27 | 7.6833 |
| E28 | 5.6076 |
| E29 | 8.2582 |
| E30 | 10.3887 |
| E31 | 8.3626 |
| E32 | 6.1085 |
| E33 | 10.4769 |
| E34 | 7.8332 |
| E35 | 7.4962 |
| E36 | 7.8793 |
| E37 | 9.665 |
| E38 | 8.5312 |
| E39 | 8.0384 |
| E40 | 7.546 |
| E41 | 6.2501 |
| E42 | 7.8581 |
| E43 | 6.1119 |
| E44 | 5.5088 |
| E45 | 6.044 |
| A (DPEOP) | 8.0468 |
| B | 3.4034 |

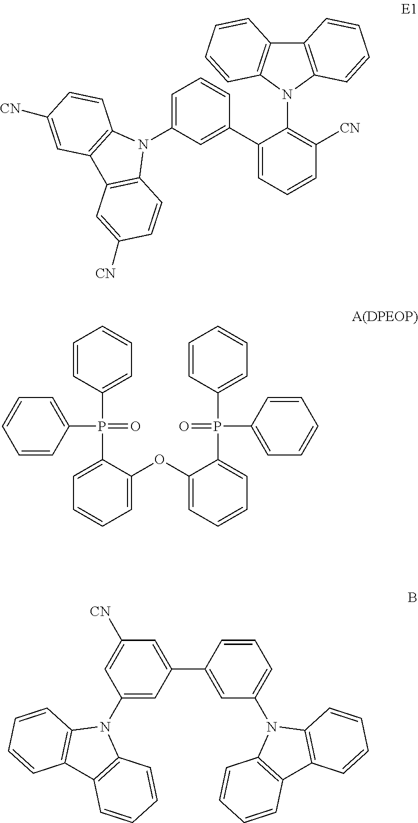

### Example 1

A glass substrate, on which a 1,500 Å ITO electrode (first electrode, anode), was formed, was washed with distilled water ultrasonic wave. When the washing with distilled water was completed, sonification washing was performed using a solvent, such as isopropyl alcohol, acetone, or methanol. The result was dried and then transferred to a plasma washer, and the resultant substrate was washed with oxygen plasma for 5 minutes and then, transferred to a vacuum depositing device.

Compound HT3 and Compound HT-D2 were co-deposited on the ITO electrode of the glass substrate to form a hole injection layer having a thickness of 100 Å, Compound HT3 was deposited on the hole injection layer to form a hole transport layer having a thickness of 1,350 Å, and mCP was deposited on the hole transport layer to form an electron blocking layer having a thickness of 100 Å, thereby forming a hole transport region.

A host and a delayed fluorescence emitter were co-deposited on the hole transport region at a volume ratio of 85:15 to form an emission layer having a thickness of 300 Å. Compound H19 (first material) and Compound E1 (second material) were used as the host at a weight ratio of 1:9, and Compound D205 was used as the delayed fluorescence emitter.

Compound BCP was vacuum-deposited on the emission layer to form a hole blocking layer having a thickness of 100 Å, Compound ET3 and LiQ were vacuum-deposited together on the hole blocking layer to form an electron transport layer having a thickness of 300 Å, and then, LiQ was deposited on the electron transport layer to form an electron injection layer having a thickness of 10 Å, and an Al second electrode (cathode) having a thickness of 1,000 Å was formed on the electron injection layer, thereby completing the manufacture of an organic light-emitting device.

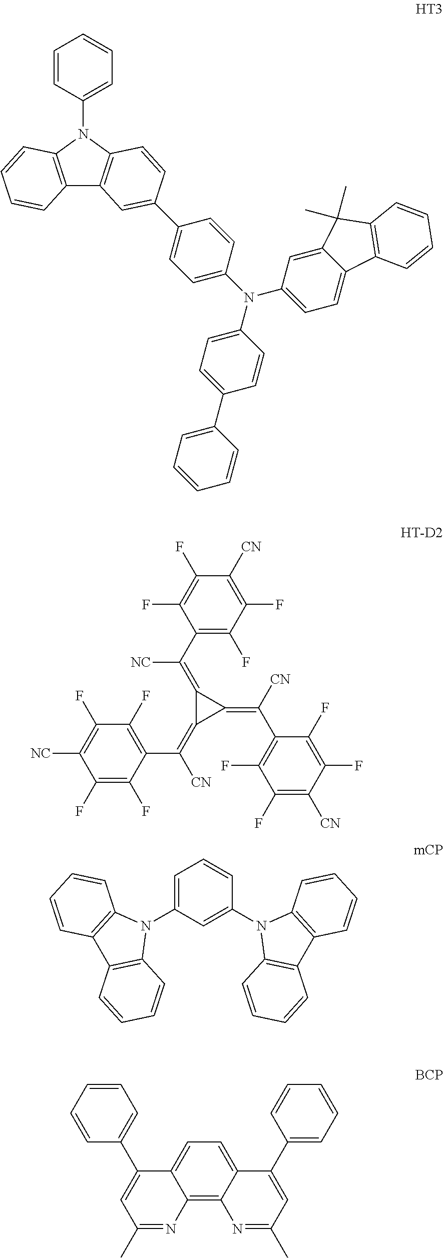 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1cc(-n2c3ccccc3c3ccccc32)cc(-n2c3ccccc3c3ccccc32)c1 ; Cc1cc(-c2ccccc2)c2ccc3c(-c4ccccc4)cc(C)nc3c2n1]

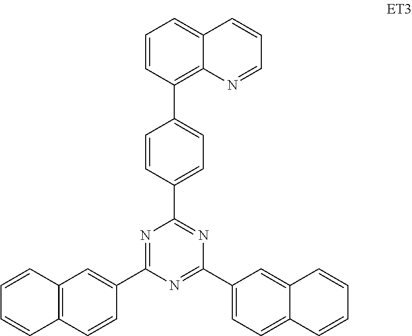 [SMILES: c1ccc2cc(-c3nc(-c4ccc(-c5cccc6cccnc56)cc4)nc(-c4ccc5ccccc5c4)n3)ccc2c1]

### Examples 2 and 3 and Comparative Examples 1-1 to 3-4

Organic light-emitting devices were manufactured in the same manner as in Example 1, except that the structure of the emission layer was changed as shown in Table 2.

### Evaluation Example 2: Evaluation of Data about Organic Light-Emitting Devices

The external quantum efficiency (EQE at 500 cd/m²) and lifespan (Too) of Examples 1 to 3 and Comparative Examples 1-1 to 3-4 were measured by using a current-voltage meter (Keithley 2400) and a luminance meter (Minolta Cs-1000A), and results thereof are shown in Table 2. The lifespan (Too) data (at 500 cd/m²) in Table 2 indicates an amount of time (hr) that lapsed when luminance was 90% of initial luminance (100%).

TABLE 2

| | Host | | | | | |
|---|---|---|---|---|---|---|
| | | | Volume ratio of | Delayed | EQE at 500 | Lifespan (T₉₀) |
| | First | Second | first material and | fluorescence | cd/m² | at 500 cd/m² |
| No. | material | material | second material | emitter | (%) | (hr) |
| Example 1 | H19 | E1 | 1:9 | D205 | 9.1 | 10.59 |
| Comparative | H19 | — | — | D205 | 7.3 | 0.33 |
| Example 1-1 | | | | | | |
| Comparative | — | E1 | — | D205 | 9.7 | 4.58 |
| Example 1-2 | | | | | | |
| Comparative | — | A | — | D205 | 9.7 | 0.07 |
| Example 1-3 | | | | | | |
| Comparative | H19 | B | 1:9 | D205 | 6.8 | 9.55 |
| Example 1-4 | | | | | | |
| Example 2 | H19 | E1 | 1:9 | D2-2 | 4.2 | 1.25 |
| Comparative | H19 | — | — | D2-2 | 3.8 | 0.31 |
| Example 2-1 | | | | | | |
| Comparative | — | E1 | — | D2-2 | 4.6 | 0.59 |
| Example 2-2 | | | | | | |
| Comparative | — | A | — | D2-2 | 5.7 | 0.06 |
| Example 2-3 | | | | | | |
| Comparative | H19 | B | 1:9 | D2-2 | 2.6 | 1.12 |
| Example 2-4 | | | | | | |
| Example 3 | H19 | E1 | 1:9 | 4 | 12.5 | 13.21 |
| Comparative | H19 | — | — | 4 | 8.8 | 0.07 |
| Example 3-1 | | | | | | |
| Comparative | — | E1 | — | 4 | 12.5 | 9.19 |
| Example 3-2 | | | | | | |
| Comparative | — | A | — | 4 | 15.2 | 0.07 |
| Example 3-3 | | | | | | |
| Comparative | H19 | B | 1:9 | 4 | 8.8 | 10.24 |
| Example 3-4 | | | | | | |

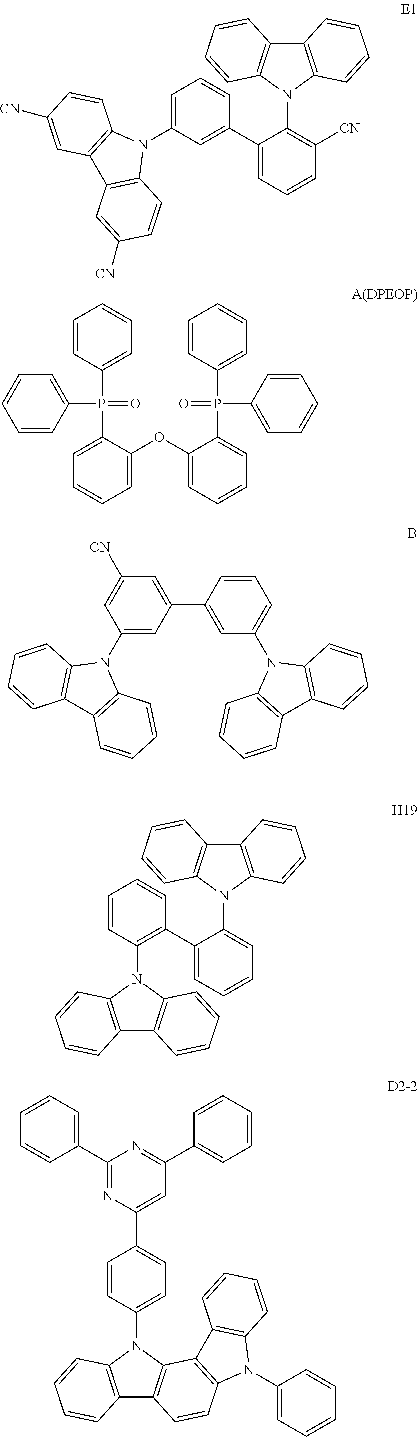

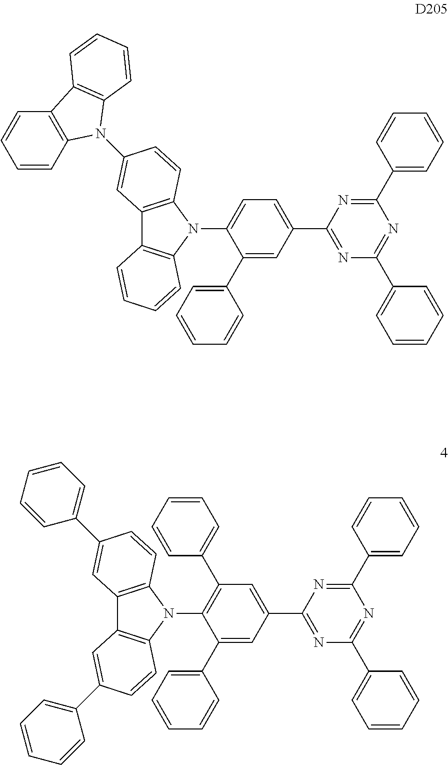 [SMILES: c1ccc(-c2nc(-c3ccccc3)nc(-c3ccc(-n4c5ccccc5c5cc(-n6c7ccccc7c7ccccc76)ccc54)c(-c4ccccc4)c3)n2)cc1 ; c1ccc(-c2ccc3c(c2)c2cc(-c4ccccc4)ccc2n3-c2c(-c3ccccc3)cc(-c3nc(-c4ccccc4)nc(-c4ccccc4)n3)cc2-c2ccccc2)cc1]

Referring to Table 2, it is confirmed that the organic light-emitting devices of Examples 1 to 3 have improved lifespan characteristics as compared with the organic light-emitting devices of Comparative Exampled 1-1 to 1-3, 2-1 to 2-3 and 3-1 to 3-3 and have improved external quantum efficiency as Comparative Examples 1-1, 1-4, 2-1, 2-4, 3-1, and 3-4. Therefore, it is confirmed that the organic light-emitting devices of Examples 1 to 3 have excellent lifespan and external quantum efficiency at the “same time”.

The organic light-emitting device may have high efficiency and a long lifespan.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

What is claimed is:

1. An organic light-emitting device comprising:
a first electrode;
a second electrode facing the first electrode; and
an emission layer between the first electrode and the second electrode,
wherein the emission layer comprises a host and a thermally activated delayed fluorescence emitter,
the host comprises a first material and a second material,
the first material and the second material are different from each other,
the second material has a dipole moment of about 5.5 debye or more,
the second material does not comprise a group represented by *^{=o}, and
* indicates a binding site to a neighboring atom.

2. The organic light-emitting device of claim 1, wherein
the second material has a dipole moment in a range of about 5.5 debye to about 15.0 debye.

3. The organic light-emitting device of claim 1, wherein
the first material comprises a π electron-rich cyclic group, and does not comprise an electron transport moiety,
the second material comprises a π electron-rich cyclic group and an electron transport moiety, and
the electron transport moiety is a cyano group, a π electron-depleted nitrogen-containing cyclic group, or a combination thereof.

4. The organic light-emitting device of claim 1, wherein
each of the first material and the second material comprises a carbazole group.

5. The organic light-emitting device of claim 1, wherein
the first material comprises a cyano group-free benzene group and a cyano group-free carbazole group, and
the second material comprises a cyano group-containing benzene group, a cyano group-containing carbazole group, or a combination thereof.

6. The organic light-emitting device of claim 1, wherein
the first material comprises a compound represented by Formula H-1(1), a compound represented by Formula H-1(2), a compound represented by Formula H-1(3), or a combination thereof: 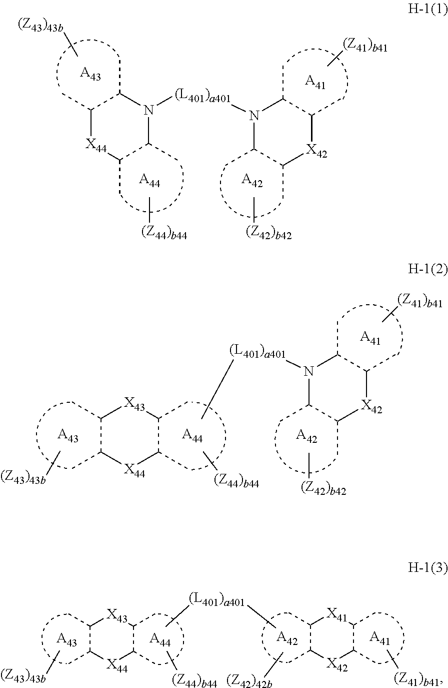
wherein, in Formulae H-1(1) to H-1(3),
ring A₄₁ to ring A₄₄ are each independently a benzene group, a naphthalene group, an indene group, an indole group, a benzofuran group, a benzothiophene group, a benzosilole group, a fluorene group, a carbazole group, a dibenzofuran group, a dibenzothiophene group, or a dibenzosilole group,
X₄₁ is N-[(L₄₁₁)_{c411}-Z₄₁₁], C(Z₄₁₅)(Z₄₁₆), O, or S,
X₄₂ is a single bond, N-[(L₄₁₂)_{c412}-Z₄₁₂], C(Z₄₁₇)(Z₄₁₈), O, or S,
X₄₃ is N-[(L₄₁₃)_{c413}-Z₄₁₃], C(Z₄₁₉)(Z₄₂₀), O, or S,
X₄₄ is a single bond, N-[(L₄₁₄)_{c414}-Z₄₁₄], C(Z₄₂₁)(Z₄₂₂), O, or S,
L₄₀₁ and L₄₁₁ to L₄₁₄ are each independently:
a single bond; or
a π electron-rich cyclic group unsubstituted or substituted with a deuterium, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, a (C₁-C₂₀ alkyl)phenyl group, —Si(Q₄₀₁)(Q₄₀₂)(Q₄₀₃), or a combination thereof,
a401 and c411 to c414 are each independently an integer from 1 to 10, wherein, when a401 is two or more, two or more L₄₀₁ are identical to or different from each other, when c411 is two or more, two or more L₄₁₁ are identical to or different from each other, when c412 is two or more, two or more L₄₁₂ are identical to or different from each other, when c413 is two or more, two or more L₄₁₃ are identical to or different from each other, and when c414 is two or more, two or more L₄₁₄ are identical to or different from each other,
Z₄₁ to Z₄₄ and Z₄₁₁ to Z₄₂₂ are each independently:
a hydrogen, a deuterium, a C₁-C₂₀ alkyl group, or a C₁-C₂₀ alkoxy group; or
a π electron-rich cyclic group unsubstituted or substituted with a deuterium, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, a (C₁-C₂₀ alkyl)phenyl group, —Si(Q₄₀₁)(Q₄₀₂)(Q₄₀₃), or a combination thereof,
b41 to b44 are each independently 1, 2, 3, or 4, and
Q₄₀₁ to Q₄₀₃ are each independently hydrogen, deuterium, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, or a tetraphenyl group.

7. The organic light-emitting device of claim 6, wherein
L₄₀₁ and L₄₁₁ to L₄₁₄ are each independently:
a single bond; or
a benzene group, a fluorene group, a dibenzofuran group, a dibenzothiophene group, a carbazole group, a dibenzosilole group, an indenocarbazole group, an indolocarbazole group, a benzofurocarbazole group, a benzothienocarbazole group, a benzosilolocarbazole group, an acridine group, or a dihydroacridine group, each unsubstituted or substituted with a deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, a (C₁-C₁₀ alkyl)phenyl group, or a combination thereof; and
Z₄₁ to Z₄₄ and Z₄₁₁ to Z₄₂₂ are each independently:
a hydrogen, a deuterium, a C₁-C₁₀ alkyl group, or a C₁-C₁₀ alkoxy group; or
a phenyl group, a biphenyl group, a terphenyl group, a tetraphenyl group, a fluorenyl group, a dibenzocarbazolyl group, a dibenzofuranyl group, a dibenzothiophenyl group, a carbazolyl group, a dibenzosilolyl group, an indenocarbazolyl group, an indolocarbazolyl group, a benzofurocarbazolyl group, a benzothienocarbazolyl group, a benzosilolocarbazolyl group, an acridinyl group, or a dihydroacridinyl group, each unsubstituted or substituted with a deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, a (C₁-C₁₀ alkyl)phenyl group, or a combination thereof.

8. The organic light-emitting device of claim 1, wherein
the second material comprises a compound represented by Formula E-1: 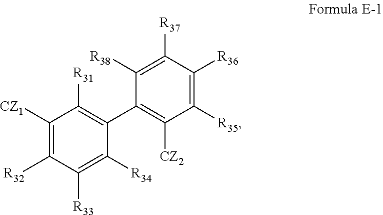
wherein, in Formula E-1, CZ₁ and CZ₂ are each independently groups represented by Formulae 2-1 or 2-2, 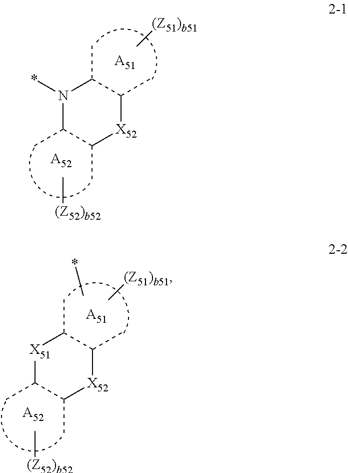
wherein, in Formulae 2-1 and 2-2, ring A₅₁ and ring A₅₂ are each independently a benzene group, a naphthalene group, an indene group, an indole group, a benzofuran group, a benzothiophene group, a benzosilole group, a fluorene group, a carbazole group, a dibenzofuran group, a dibenzothiophene group, a dibenzosilole group, a pyridine group, a pyrimidine group, a pyrazine group, a pyridazine group, a triazine group, a quinoline group, an isoquinoline group, a quinoxaline group, a quinazoline group, a pyrazole group, an imidazole group, a triazole group, an oxazole group, an isoxazole group, a thiazole group, an isothiazole group, an oxadiazole group, a thiadiazole group, an azaindene group, an azaindole group, an azabenzofuran group, an azabenzothiophene group, an azabenzosilole group, an azafluorene group, an azacarbazole group, an azadibenzofuran group, an azadibenzothiophene group, or an azadibenzosilole group,
in Formula 2-2, X₅₁ is N-[(L₅₁₁)_{c511}-Z₅₁₁], C(Z₅₁₃)(Z₅₁₄), O, or S,
in Formulae 2-1 and 2-2, X₅₂ is a single bond, N-[(L₅₁₂)_{c512}-Z₅₁₂], C(Z₅₁₅)(Z₅₁₆), O, or S,
in Formula 2-2, i) X₅₁ is N-[(L₅₁₁)_{c511}-Z₅₁₁], or ii) ring A₅₁, ring A₅₂, or a combination thereof is an indole group, a carbazole group, an azaindole group, or an azacarbazole group,
L₅₁₁ and L₅₁₂ are each independently a single bond, a substituted or unsubstituted C₃-C₁₀ cycloalkylene group, a substituted or unsubstituted C₂-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₂-C₆₀ heteroarylene group, a substituted or unsubstituted divalent non-aromatic condensed polycyclic group, or a substituted or unsubstituted divalent non-aromatic condensed heteropolycyclic group,
c511 and c512 are each independently an integer from 1 to 10, wherein, when c511 is two or more, two or more L₅₁₁ are identical to or different from each other, and when c512 is two or more, two or more L₅₁₂ are identical to or different from each other,
Z₃₁ to Z₃₈, Z₅₁, Z₅₂, and Z₅₁₁ to Z₅₁₆ are each independently:
a hydrogen, a deuterium, or a cyano group (CN); or
a C₁-C₂₀ alkyl group, a phenyl group, a biphenyl group, a terphenyl group, a dibenzofuranyl group, or a dibenzothiophenyl group, each unsubstituted or substituted with a deuterium, a cyano group, a C₁-C₂₀ alkyl group, a phenyl group, a biphenyl group, or a combination thereof,
in Formulae 2-1 and 2-2, * indicates a binding site to a neighboring atom,
the number of cyano groups included in Formula E-1 is 1 or more,
a substituent of the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₂-C₆₀ heteroarylene group, the substituted divalent non-aromatic condensed polycyclic group, the substituted divalent non-aromatic condensed heteropolycycic group, or a combination thereof is:
a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a combination thereof;
a C₁-C₆₀ alkyl group, a C₂-C₆₀ alkenyl group, a C₂-C₆₀ alkynyl group, and a C₁-C₆₀ alkoxy group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), —B(Q₁₆)(Q₁₇), or a combination thereof;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, or a combination thereof;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic condensed heteropolycycic group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C3-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), —B(Q₂₆)(Q₂₇), or a combination thereof; or
—Si(Q₃₁)(Q₃₂)(Q₃₃), —N(Q₃₄)(Q₃₅), —B(Q₃₆)(Q₃₇), or a combination thereof, and
Q₁ to Q₇, Q₁₁ to Q₁₇, Q₂₁ to Q₂₇, and Q₃₁ to Q₃₇ are each independently a hydrogen, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, or a substituted or unsubstituted monovalent non-aromatic condensed heteropolycyclic group.

9. The organic light-emitting device of claim 8, wherein
CZ₁ and CZ₂ are each independently one of groups represented by Formulae 2(1) to 2(3): 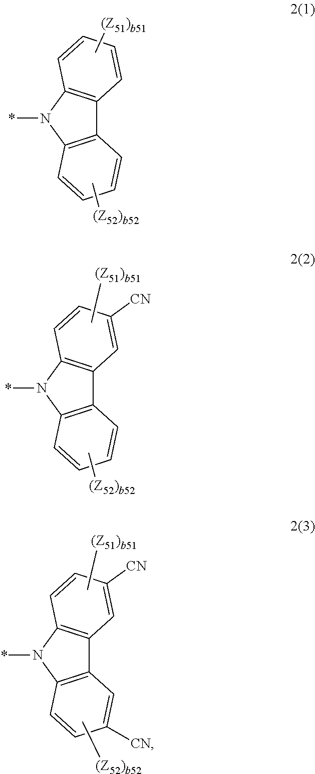
wherein, in Formulae 2(1) to 2(3), Z₅₁ and Z₅₂ are each independently the same as described in claim 8, b51, and b52 are each independently 0, 1, or 2, and * indicates a binding site to a neighboring atom.

10. The organic light-emitting device of claim 8, wherein
at least one of R₃₁ to R₃₈ is a cyano group.

11. The organic light-emitting device of claim 1, wherein
a difference between a triplet energy level of the thermally activated delayed fluorescence emitter and a singlet energy level of the thermally activated delayed fluorescence emitter is in a range of about 0 to about 0.5 electron volt, and
the triplet energy level and the singlet energy level are evaluated by using a density functional theory method structurally optimized at a level of B3LYP/6-31G(d,p).

12. The organic light-emitting device of claim 1, wherein
the thermally activated delayed fluorescence emitter comprises a compound represented by Formula 11: 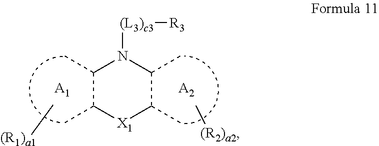
wherein, in Formula 11,
X₁ is a single bond, N-[(L₄)_{c4}-R₄], C(R₅)(R₆), O, or S,
ring A₁ and ring A₂ are each independently a benzene group, a naphthalene group, an indene group, an indole group, a benzofuran group, a benzothiophene group, a benzosilole group, a fluorene group, a carbazole group, a dibenzofuran group, a dibenzothiophene group, or a dibenzosilole group,
L₃ and L₄ are each independently a single bond, a substituted or unsubstituted C₃-C₁₀ cycloalkylene group, a substituted or unsubstituted C₂-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₂-C₆₀ heteroarylene group, a substituted or unsubstituted divalent non-aromatic condensed polycyclic group, or a substituted or unsubstituted divalent non-aromatic condensed heteropolycyclic group,
c3 and c4 are each independently an integer from 1 to 4, wherein, when c3 is two or more, two or more L₃ are identical to or different from each other, and when c4 is two or more, two or more L₄ are identical to or different from each other,
R₁ to R₆ are each independently a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), or —B(Q₆)(Q₇),
a1 and a2 are each independently an integer from 0 to 10,
a substituent of the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₂-C₆₀ heteroarylene group, the substituted divalent non-aromatic condensed polycyclic group, the substituted divalent non-aromatic condensed heteropolycyclic group, the substituted C₆-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic condensed heteropolycyclic group, or a combination thereof is:
a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁s), —B(Q₁a)(Q₁₇), or a combination thereof;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, or a monovalent non-aromatic condensed heteropolycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic condensed heteropolycycic group, each substituted with a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), —B(Q₂₆)(Q₂₇), or a combination thereof; or
—Si(Q₃1)(Q₃₂)(Q₃₃), —N(Q₃₄)(Q₃₅), or —B(Q₃₆)(Q₃₇), and
Q₁ to Q₇, Q₁₁ to Q₁₇, Q₂₁ to Q₂₇, and Q₃₁ to Q₃₇ are each independently a hydrogen, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, or a substituted or unsubstituted monovalent non-aromatic condensed heteropolycyclic group.

13. The organic light-emitting device of claim 12, wherein
R₃ comprises a π electron-depleted nitrogen-containing cyclic group.

14. The organic light-emitting device of claim 12, wherein
R₃ is:
a group represented by Formula 13(1) or 13(2);
a phenyl group, a biphenyl group, a terphenyl group, a naphthyl group, a fluorenyl group, a quinolinyl group, an isoquinolinyl group, a carbazolyl group, a dibenzofuranyl group, a dibenzothiophenyl group, or an indolocarbazolyl group; or
a phenyl group, a biphenyl group, a terphenyl group, a naphthyl group, a fluorenyl group, a quinolinyl group, an isoquinolinyl group, a carbazolyl group, a dibenzofuranyl group, a dibenzothiophenyl group, or an indolocarbazolyl group, each substituted with a deuterium, a C₁-C₁₀ alkyl group, a C₁-C₁₀ alkoxy group, a phenyl group, a biphenyl group, a terphenyl group, a naphthyl group, a fluorenyl group, a pyridinyl group, a pyrimidinyl group, a pyrazinyl group, a pyridazinyl group, a triazinyl group, a quinolinyl group, an isoquinolinyl group, a carbazolyl group, a dibenzofuranyl group, a dibenzothiophenyl group, an indolocarbazolyl group, —Si(Q₃₁)(Q₃₂)(Q₃₃), or a combination thereof; and
Q₃₁ to Q₃₃ are each independently a C₁-C₁₀ alkyl group, a C₁-C₁₀ alkoxy group, a phenyl group, a biphenyl group, a terphenyl group, or a naphthyl group: 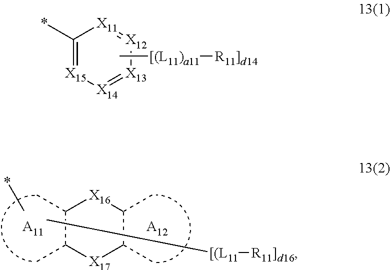
wherein, in Formulae 13(1) and 13(2),
X₁ to X₁₅ are each independently C or N, and at least one of X₁₁ to X₁₅ is N, ring A₁₁ and ring A₁₂ are each independently a benzene group, a naphthalene group, a pyridine group, a pyrazine group, a pyrimidine group, a pyridazine group, a quinoline group, an isoquinoline group, a quinoxaline group, or a quinazoline group, and ring A₁₁, ring A₁₂, or a combination thereof is a pyridine group, a pyrazine group, a pyrimidine group, a pyridazine group, a quinoline group, an isoquinoline group, a quinoxaline group, or a quinazoline group,
X₁₆ is N-[(L₁₂)ₐ₁₂-R₁₂], C(R₁₄)(R₁₅), O, or S,
X₁₇ is a single bond, N-[(L₁₃)ₐ₁₃-R₁₃], C(R₁₆)(R₁₇), O, or S,
L₁₁ to L₁₃ are each independently the same as described in connection with L₃ in claim 12,
a11 to a13 are each independently the same as described in connection with c3 in claim 12,
R₁₁ to R₁₇ are each independently the same as described in connection with R₁ in claim 12,
d16 is an integer from 0 to 6,
d14 is an integer from 0 to 4, and
* indicates a binding site to a neighboring atom.

15. The organic light-emitting device of claim 12, wherein
R₃ is a group represented by one of Formulae 13-1 to 13-20: 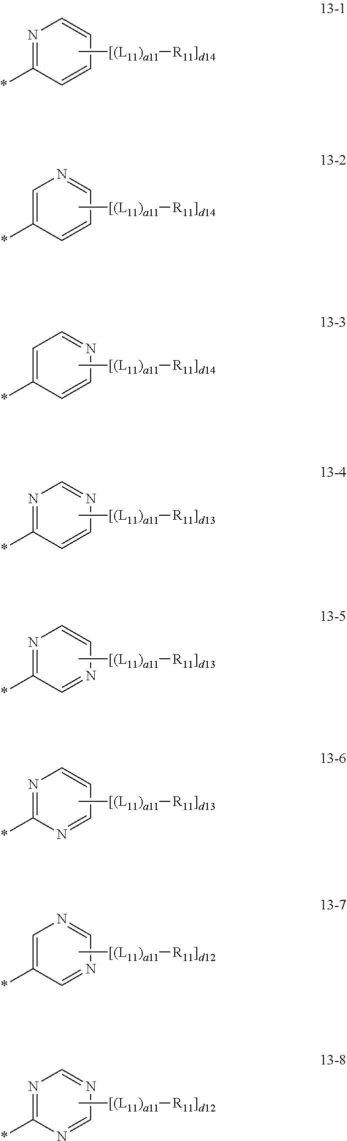 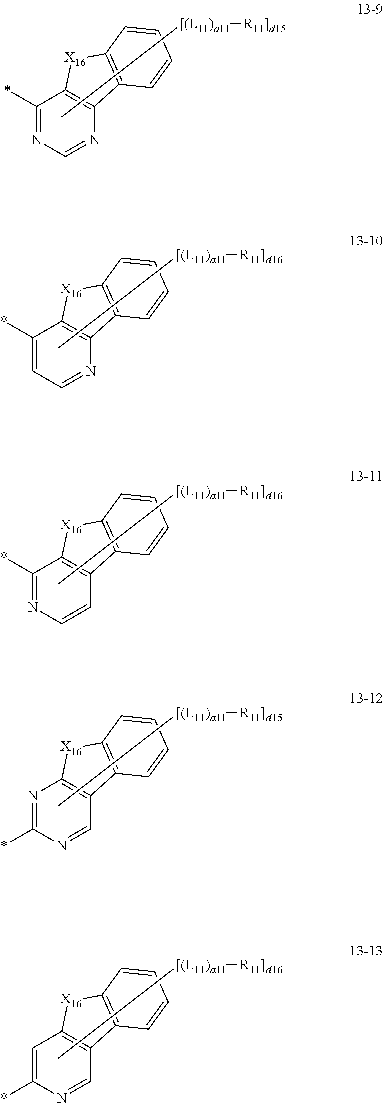 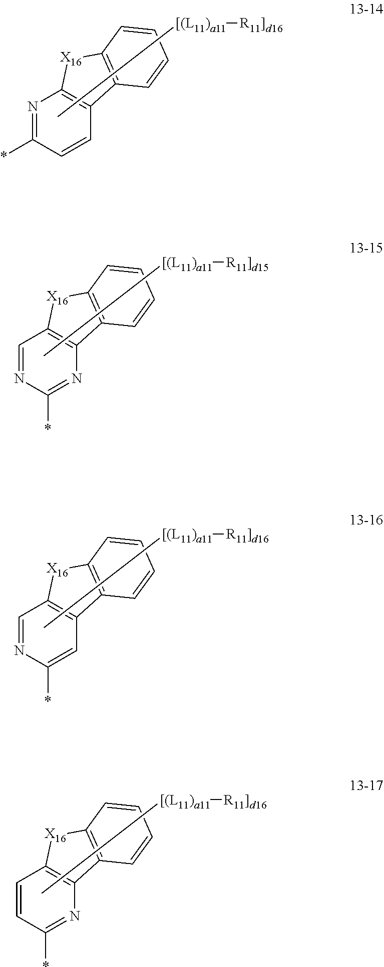 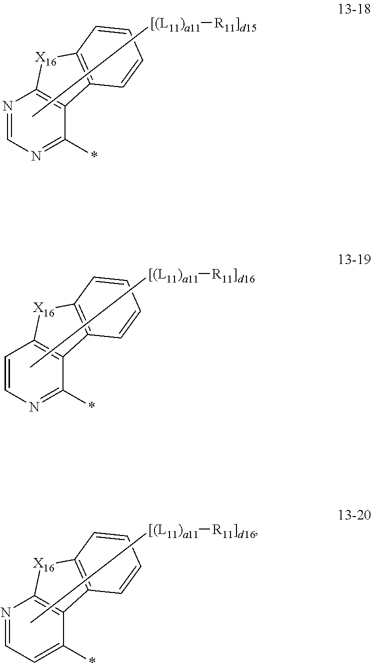
wherein, in Formulae 13-1 to 13-20,
X₁₆ is N-[(L₁₂)ₐ₁₂-R₁₂], C(R₁₄)(R₁₅), O, or S,
L₁₁ and L₁₂ are each independently the same as described in connection with L₃ in claim 12,
a11 and a12 are each independently the same as described in connection with c3 in claim 12,
R₁₁, R₁₂, R₁₄, and R₁₅ are each independently the same as described in connection with R₁ in claim 12,
d16 is an integer from 0 to 6,
d15 is an integer from 0 to 5,
d14 is an integer from 0 to 4,
d13 is an integer from 0 to 3,
d12 is an integer from 0 to 2, and
* indicates a binding site to a neighboring atom.

16. The organic light-emitting device of claim 12, wherein
the thermally activated delayed fluorescence emitter comprises a compound represented by one of Formulae 11-1 to 11-7: 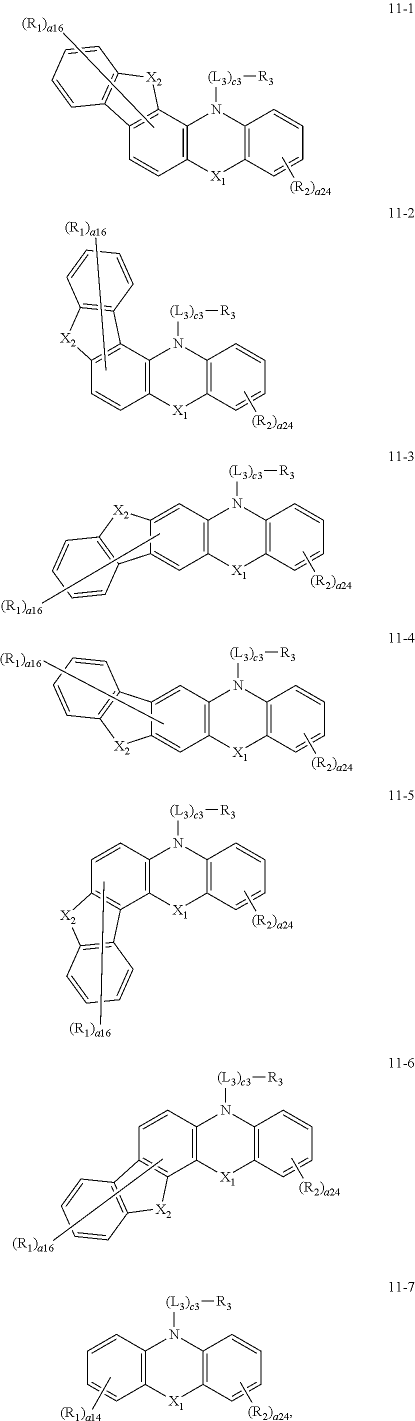
wherein, in Formulae 11-1 to 11-7,
X₁, L₃, c3, and R₁ to R₃ are each independently the same as described in claim 12,
X₂ is N-[(L₅)_{c5}-R₇], C(R₈)(R₉), O, or S,
L₅ and c5 are each independently the same as described in connection with L₃ and c3 in claim 12,
R₇ is the same as described in connection with R₃ in claim 12,
R₈ and R₉ are each independently the same as described in connection with R₅ and R₆ in claim 12, respectively,
a16 is an integer from 0 to 6, and
a14 and a24 are each independently an integer from 0 to 4.

17. The organic light-emitting device of claim 1, wherein
the emission layer does not comprise a transition metal-containing organometallic compound, and
a delayed fluorescence component emitted from the thermally activated delayed fluorescence emitter is about 90% or more based on a total emission component emitted from the emission layer.

18. The organic light-emitting device of claim 1, wherein
light emitted from the thermally activated delayed fluorescence emitter in the emission layer is blue light.

19. The organic light-emitting device of claim 1, wherein
an amount of the thermally activated delayed fluorescence emitter is in a range of about 0.01 parts by weight to about 30 parts by weight based on 100 parts by weight of the host.

20. The organic light-emitting device of claim 1, wherein
an amount of the first material in the emission layer is larger than an amount of the second material.
